(19) United States
(12) Patent Application Publication
(10) Pub. No.: US 2024/0366680 A1
(43) Pub. Date: Nov. 7, 2024

(54) TREATMENT OF PAIN

(71) Applicant: Editas Medicine, Inc., Cambridge, MA (US)

(72) Inventors: Christopher Wilson, Cambridge, MA (US); Shyam Ramachandran, Winchester, MA (US)

(21) Appl. No.: 18/683,967

(22) PCT Filed: Aug. 15, 2022
(86) PCT No.: PCT/US22/40320

§ 371 Date: Feb. 15, 2024

Related U.S. Application Data
(60) Provisional application No. 63/233,445, filed on Aug. 16, 2021.

(51) Int. Cl. A61K 35/30; A61K 31/7105; A61K 38/46; C12N 9/22; C12N 15/11; C12N 15/90

(52) U.S. Cl. CPC A61K 35/30; A61K 31/7105; A61K 38/465; C12N 9/22; C12N 15/11; C12N 15/907; C12N 2310/20

(57) ABSTRACT

The present application provides technologies and methodologies comprising gene editing for treatment of one or more symptoms of a pain disorder and/or disorder associated with pain. Wherein an AAV vector comprising a nucleotide sequence encoding two or more gRNAs is implemented, and further wherein the two or more gRNAs comprise a spacer comprising specified nucleotide sequence.

### CROSS-REFERENCE TO RELATED APPLICATIONS

This application claims the benefit of U.S. Provisional Application No. 63/233,445, filed Aug. 16, 2021, the contents of which are hereby incorporated herein by reference in their entirety.

### BACKGROUND

CRISPRs (Clustered Regularly Interspaced Short Palindromic Repeats) evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a site-specific nuclease protein such as Cas9 or Cpf1 to a target sequence in the viral genome. The RNA-guided nuclease, in turn, cleaves and thereby silences the viral target.

CRISPR systems have been adapted for gene editing in eukaryotic cells. These systems generally include a protein component (the RNA-guided nuclease) and a nucleic acid component (generally referred to as a guide RNA or “gRNA”). These two components form a complex that interacts with specific target DNA sequences recognized by, or complementary to, the two components of the system and optionally edits or alters the target sequence, for example by means of site-specific DNA cleavage. The value of nucleases such as these as a tool for the treatment of many diseases is widely recognized.

### SUMMARY

In one aspect, the present disclosure provides a gRNA comprising a spacer comprising the nucleotide sequence of any one of SEQ ID NOs:1-310.

In one aspect, the present disclosure provides a gRNA, wherein the spacer consists of the nucleotide sequence of any one of SEQ ID NOs:1-310.

In one aspect, the present disclosure provides a composition comprising a gRNA comprising any one of SEQ ID NOs.: 1-310.

In one aspect, the present disclosure provides an AAV vector comprising the nucleotide sequence of any one of SEQ ID NOs:1-310.

In one aspect, the present disclosure provides an AAV vector comprising a nucleotide sequence encoding two or more gRNAs, wherein the two or more gRNAs comprise a spacer comprising the nucleotide sequence of any one of SEQ ID NOs:1-310.

In one aspect, the present disclosure provides an AAV vector comprising two or more nucleotide sequences, wherein the two or more nucleotide sequences encode two or more gRNAs comprising a spacer comprising the nucleotide sequence of any one of SEQ ID NOs:1-310.

In one aspect, the present disclosure provides an AAV vector comprising a first nucleotide sequence encoding a gRNA comprising a spacer comprising the nucleotide sequence of SEQ ID NO:5, and a second nucleotide sequence encoding a gRNA comprising a spacer comprising the nucleotide sequence of SEQ ID NO:189.

In one aspect, the present disclosure provides an AAV vector comprising a first nucleotide sequence encoding a gRNA comprising a spacer comprising the nucleotide sequence of SEQ ID NO:7, and a second nucleotide sequence encoding a gRNA comprising a spacer comprising the nucleotide sequence of SEQ ID NO:182.

In one aspect, the present disclosure provides an AAV vector comprising a first nucleotide sequence encoding a gRNA comprising a spacer comprising the nucleotide sequence of SEQ ID NO:7, and a second nucleotide sequence encoding a gRNA comprising a spacer comprising the nucleotide sequence of SEQ ID NO:189.

In one aspect, the present disclosure provides an AAV vector comprising a first nucleotide sequence encoding a gRNA comprising a spacer comprising the nucleotide sequence of SEQ ID NO:5, and a second nucleotide sequence encoding a gRNA comprising a spacer comprising the nucleotide sequence of SEQ ID NO:182.

In some embodiments, the AAV vector which further comprises a nucleotide sequence encoding an RNA-guided nuclease. In some such embodiments, the cell comprises the AAV vector.

In one aspect, the present disclosure provides a method comprising administering to a subject in need thereof (i) a first nucleotide sequence that encodes a guide RNA (gRNA) that targets a gene encoding a voltage-gated sodium channel subunit, and (ii) a second nucleotide sequence that encodes an RNA-guided nuclease, wherein the gRNA comprises a spacer comprising or consisting of the nucleotide sequence of any one of SEQ ID NOs:1-310.

In one aspect, the present disclosure provides a method comprising: administering to a subject in need thereof a composition comprising (i) a gRNA that targets a gene encoding a voltage-gated sodium channel subunit, wherein the gRNA comprises a spacer comprising or consisting of the nucleotide sequence of any one of SEQ ID NOs:1-310; and (ii) an RNA-guided nuclease.

In some embodiments, the subject has or is at risk of a pain disorder and/or a disorder associated with pain.

In some embodiments, the subject has a genetic mutation within the SCN9A and/or SCN10A locus.

In some embodiments, the pain disorder is nociceptive and/or neuropathic pain.

In one aspect, the present disclosure provides a method of treating a subject for one or more symptoms associated with a pain disorder, the method comprising administering to a subject in need thereof (i) a first nucleotide sequence that encodes a gRNA that targets a gene encoding a voltage-gated sodium channel subunit, and (ii) a second nucleotide sequence that encodes an RNA-guided nuclease, wherein the gRNA comprises a spacer comprising or consisting of the nucleotide sequence of one of SEQ ID NOs:1-310, e.g., wherein a level or measure of at least one symptom is reduced or ameliorated relative to control (e.g., a level or measure of the at least one symptom in the subject prior to the administering).

In one aspect, the present disclosure provides a method of treating a subject for one or more symptoms associated with a pain disorder, the method comprising: administering to a subject in need thereof a composition comprising (i) a gRNA that targets a gene encoding a voltage-gated sodium channel subunit, wherein the gRNA comprises a spacer comprising or consisting of the nucleotide sequence of any one of SEQ ID NOs:1-310; and (ii) an RNA-guided nuclease, wherein a level or measure of at least one symptom is reduced or ameliorated relative to control (e.g., a level or measure of the at least one symptom in the subject prior to the administering).

In some embodiments, the method comprises administering a complex of the gRNA and the RNA-guided nuclease. In some embodiments, the method comprises administering an RNP complex of the gRNA and the RNA-guided nuclease.

In some embodiments, the method comprises a subject, wherein the subject has a genetic mutation within the SCN9A and/or SCN10A locus.

In some embodiments, the method comprises a subject, wherein the subject has or is at risk of a pain disorder and/or disorder associated with pain.

In one aspect, the present disclosure provides a method of treating a subject, the method comprising administering to a neuronal cell of a subject in need thereof (i) a first nucleotide sequence that encodes a gRNA that targets a gene encoding a voltage-gated sodium channel subunit, and (ii) a second nucleotide sequence that encodes an RNA-guided nuclease, wherein the gRNA comprises a spacer comprising or consisting of the nucleotide sequence of one of SEQ ID NOs:1-310.

In some embodiments, the administering comprises contacting at least a portion of a neuronal cell with the first and/or second nucleotide sequences.

In some embodiments, the neuronal cell is a neuron or progenitor thereof. In some embodiments, the neuronal cell or progenitor is a dorsal root ganglion or dorsal root ganglion cell progenitor.

In one aspect, the present disclosure provides a method comprising administering to a neuronal cell of a subject in need thereof (i) a gRNA that targets a gene encoding a voltage-gated sodium channel subunit, and (ii) an RNA-guided nuclease, wherein the gRNA comprises a spacer comprising or consisting of the nucleotide sequence of one of SEQ ID NOs:1-310.

In some embodiments, the method comprises administering a complex of the gRNA and the RNA-guided nuclease.

In some embodiments, the method comprises administering an RNP complex of the gRNA and the RNA-guided nuclease.

In some embodiments, the administering comprises contacting at least a portion of the neuronal cell with the gRNA and the RNA-guided nuclease.

In some embodiments, the neuronal cell is a neuron or progenitor thereof. In some embodiments, the neuronal cell or progenitor is a dorsal root ganglion or dorsal root ganglion cell progenitor.

In some embodiments, the neuronal cell has a genetic mutation within the SCN9A or SCN10A locus.

In one aspect, the present disclosure provides a method of editing a neuronal cell in a subject in need thereof, the method comprising: administering to the subject (i) a first nucleotide sequence that encodes a gRNA that targets a gene encoding a voltage-gated sodium channel subunit, wherein the gRNA comprises a spacer comprising or consisting of the nucleotide sequence of any one of SEQ ID NOs:1-310; and (ii) a second nucleotide sequence that encodes an RNA-guided nuclease, wherein upon expression of the gRNA and the RNA-guided nuclease, the nuclease creates one or more single- or double-strand breaks within the gene or within a regulatory element of the gene.

In some embodiments, the one or more breaks results in one or more nucleotide changes in the gene or regulatory element of the gene.

In some embodiments, the level of expression of the gene is changed relative to control.

In some embodiments, the gene is SCN9A and/or SCN10A.

In some embodiments, the one or more changes is a permanent insertion, deletion, and/or substitution of at least one nucleotide within or near the SCN9A and/or SCN10A gene(s) or regulatory element(s). In some embodiments, the change in gene expression is a reduction in expression.

In some embodiments, the level of SCN9A and/or SCN10A gene expression is reduced as compared to control.

In one aspect, the present disclosure provides a method of editing a neuronal cell in a subject in need thereof comprising: administering to the subject (i) a first nucleotide sequence that encodes a gRNA that targets an SCN9A and/or SCN10A gene or regulatory element, wherein the gRNA comprises a spacer comprising or consisting of the nucleotide sequence of any one of SEQ ID NOs:1-310; and (ii) a second nucleotide sequence that encodes an RNA-guided nuclease, wherein upon expression of the gRNA and the RNA-guided nuclease, the nuclease creates one or more single- or double-strand breaks within the SCN9A or SCN10A genes or regulatory element.

In one aspect, the present disclosure provides a method of treating a subject, the method comprising: (i) administering to an autologous iPSC cell (a) a first nucleotide sequence that encodes a gRNA that targets a gene encoding a voltage-gated sodium channel subunit, wherein the gRNA comprises a spacer comprising or consisting of the nucleotide sequence of any one of SEQ ID NOs:1-310; and (b) a second nucleotide sequence that encodes an RNA-guided nuclease; (ii) differentiating the iPSC cell into a neuron or neuronal progenitor; and (iii) transplanting the differentiated cell into a subject.

In some embodiments, the present disclosure provides a gRNA that targets a PAM that is targeted by a gRNA comprising a spacer comprising the nucleotide sequence of any one of SEQ ID NOs:1-310.

In one aspect, the present disclosure provides a gRNA that targets (i) a target sequence of a gene encoding a voltage-gated sodium channel subunit or (ii) a target sequence of a regulatory element of the gene, wherein the target sequence is targeted by a gRNA comprising a spacer comprising the nucleotide sequence of any one of SEQ ID NOs:1-310.

In some embodiments, the method comprises administering a vector comprising the first nucleotide sequence and/or the second nucleotide sequence.

In some embodiments, the vector is an AAV vector. In some embodiments, the AAV vector comprises components from one serotype of AAV. In some embodiments, the AAV vector comprises components from more than one serotype of AAV. In some embodiments, the serotype is 1, 2, 3, 4, 5, 6, 7, 8, or 9.

In some embodiments, a cell is a neuronal cell or precursor thereof. In some embodiments the neuronal cell is a peripheral neuronal cell or precursor thereof. In some embodiments, the peripheral neuronal cell is a dorsal root ganglion cell.

In some embodiments, the composition further comprises an RNA-guided nuclease. In some embodiments, the composition further comprises a complex of a gRNA and an RNA-guided nuclease. In some embodiments, the composition comprises an RNP complex of the gRNA and the RNA-guided nuclease.

In some embodiments, the composition comprises an AAV vector. In some embodiments, the composition further comprises a second AAV vector comprising a nucleotide sequence encoding an RNA-guided nuclease.

### BRIEF DESCRIPTION OF THE DRAWING

FIG. 1. depicts the transduction efficiency of various AAV serotypes for neurons, at different dosages in human donor Dorsal Root Ganglion (DRG) explants. The bar graphs represent the percentage neuronal cell transduction efficiency averaged across explants from multiple donors per serotype-dose condition while the error bars represent the standard deviation. Each condition is represented by a population of explants from 4 donors. For each donor, four explants were used per condition, and 3 slides were prepared each explant resulting in 24 sections for imaging each condition. The neurons were identified in the explant sections using IHC staining for Tuj 1.

FIG. 2 depicts the transduction efficiency of various AAV serotypes for nociceptors as defined by neurons showing peripherin-high staining, at different dosages in human donor Dorsal Root Ganglion (DRG) explants. The bar graphs represent the percentage nociceptor transduction efficiency averaged across explants from multiple donors per serotype-dose condition while the error bars represent the standard deviation. Each condition is represented by a population explants from 4 donors. For each donor, four explants were used per condition, and 3 slides were prepared and stained for each explant resulting in 24 sections for imaging each condition. The nociceptors were identified in the explant sections by using IHC staining for Tuj 1 and peripherin and selecting cells that showed Tuj1 positive and peripherin-high staining.

FIG. 3 depicts the mean GFP intensity for human donor Dorsal Root Ganglion (DRG) explant neurons transduced with various AAV serotypes at different dosages obtained from multiple donors. Mean GFP intensity acts as a measure for transduction intensity and AAV construct transcription and translational efficiency. The mean GFP intensity (arbitrary units relative to control) for individual transduced neurons was determined (represented by individual dots) and dot plots were created to represent the population distribution. The mean of the distributions across all samples for each respective condition is noted by the horizontal bar crossing the dot plot. Each condition is represented by a population of explants from 4 donors. For each donor, four explants were used per condition, and 3 slides were prepared each explant resulting in 24 sections for imaging each condition. The condition labelled “All” represents a negative control, wherein explants were transduced with AAV-eGFP and stained with only a secondary antibody. The condition labelled “N/A” represents a transduction control, wherein explants were not transduced but were stained with both primary and secondary antibodies. Sample distributions were tested for normality using the Shapiro-Wilks test, statistical analysis was then performed using the Kruskal-Wallis one-way analysis of variance. Significant differences are represented by asterisk.

FIG. 4 depicts the mean GFP intensity for human donor Dorsal Root Ganglion (DRG) explant nociceptors transduced with various AAV serotypes at different dosages obtained from multiple donors. Mean GFP intensity acts as a proxy for transduction intensity and AAV construct transcription and translational efficiency. The mean GFP intensity (arbitrary units relative to control) for individual transduced neurons was determined (represented by individual dots) and dot plots were created to represent the population distribution. The mean of the distributions across all samples for each respective condition is noted by the horizontal bar crossing the dot plot. Each condition is represented by a population of explants from 4 donors. For each donor, four explants were used per condition, and 3 slides were prepared each explant resulting in 24 sections for imaging each condition. The nociceptors were identified in the explant sections by using IHC staining for Tuj 1 and peripherin and selecting cells that showed Tuj 1 positive and peripherin-high staining. The condition labelled “All” represents a negative control, wherein explants were transduced with AAV-eGFP and stained with only a secondary antibody. The condition labelled “N/A” represents a transduction control, wherein explants were not transduced but were stained with both primary and secondary antibodies. Sample distributions were tested for normality using the Shapiro-Wilks test, statistical analysis was then performed using the Kruskal-Wallis one-way analysis of variance. Significant differences are represented by asterisk.

FIG. 5 depicts results of an AAV Serotype infection of non-neuronal cell types found within the DRG. The results of this analysis allow the calculation of the preferential infectivity analysis. The preferential infectivity analysis was calculated for each Serotype by dividing the average percentage transduced nociceptors by the average percentage transduced non-neuronal cells in human donor DRG explants. Mean transduction efficiency was determined for nociceptors as described in FIG. 2. Mean transduction efficiency was then determined for non-neuronal cells, and the preferential infectivity was calculated (fold nociceptor/non-neuronal). Each condition is represented by Each condition is represented by a population of explants from 3 donors. For each donor, there were 4 explants used per condition wherein 3 slides were prepared for each explant, resulting in 24 sections for imaging for each condition.

FIG. 6 depicts an in-vivo lateral cross section of the human thorax with a focus on the spinal cord at the L3-L4 spinal segment. The preferred site of injection is the DRG, as depicted by the injection needle. Image adapted from Bicket et al., 2017.

FIG. 7 depicts indel creation frequency in human DRG explants, as determined by drop-off RT-ddPCR. Each data point represents a single RNA sample generated from two DRG explants transduced under the same conditions. Individual human donors are labeled by color, and 2-3 samples were analyzed per donor with each virus/dosage combination. The displayed value on the graph represents median indel percentage per column. Samples that were not transduced, or were treated with AAV5 encoding Cas9 and non-targeting guides, showed minimal editing (data not shown). Samples in each virus/dosage combination were compared to controls (untransduced and non-targeting guide transduced samples) by two-tailed Student's t-test. ***=p<0.001

FIG. 8 depicts the types of indels generated in human DRG explants treated with AAV5-CMV-Cas9-guides, as determined by targeted RNA-sequencing. RNA samples were generated from two DRG explants transduced under the same conditions from an individual donor. Two or three samples were analyzed per donor (n=7 donors) with each virus/dosage combination. Graphed value represents percentage of sequenced transcripts containing indels, averaged across donors. Transcripts were classified and labeled as having a frameshift mutation (blue), having an in-frame mutation (orange), or containing multiple indels (red). Values on the graph represent the percentage in-frame indels generated out of total indels for each sequence at 3×10¹¹VG/ml.

FIG. 9A and FIG. 9B depict expression of SCN9A or SCN10A relative to TRPV1 determined by RT-ddPCR. Each data point represents a single RNA sample generated from two DRG explants transduced under the same conditions. Individual human donors are labeled by color, and 2-3 samples were analyzed per donor with each virus/dosage combination. For each sample, the concentration (copies/μl) was determined for SCN9A and SCN10A. To normalize to the abundance of nociceptors within each sample, the concentration of SCN9A or SCN10A was divided by the concentration of TRPV1, a gene specifically expressed in nociceptive neurons. The non-highlighted value in each column represents the median of this ratio across samples treated with a specific guide/AAV dosage. Control samples include both untransduced samples and samples transduced with AAV5-Cas9 encoding non-targeting guides. Ratios within each column were compared to ratios in control group with a two-tailed Student's t-test. The value highlighted in yellow represents a calculation of percentage of transcripts lost to nonsense mediated decay, calculated by dividing the difference in median ratios between control and treatment groups by median control group ratio. *=p<0.05; **=p<0.01; ***=p<0.001

FIG. 10 depicts calculations of percentage presumed loss of function edits in human DRG explants treated with AAV5-CMV-Cas9-guide at 3×10¹¹VG/ml. Percentage loss of function edits was calculated by combining percentage of transcripts lost to nonsense mediated decay with transcripts containing frameshift mutations. Percentage lost to nonsense mediated decay (column B) was determined by RT-ddPCR, as described in FIG. 9. Percentage indels created (Column C) was determined by drop-off RT-ddPCR, as described in FIG. 7. Percentage of indels which produce a frame shift (Column D) was determined by targeted RNA-sequencing, as shown in FIG. 8. The percentage of transcripts in the sample which contain a frameshift mutation (Column E) was calculated by multiplying columns C and D (percentage transcripts with indels*percentage of indel transcripts with frameshift mutation). Because the pool of transcripts analyzed by RT-ddPCR and targeted RNA-sequencing methods does not include transcripts which were degraded due to nonsense mediated decay, the percentage of transcripts containing frameshift mutations as a proportion of total pool of transcripts, including both transcripts which are still present and those lost to nonsense mediated decay, was calculated (Column F), by multiplying the percentage of the total pool of RNA which has not been degraded (100%-Column B) by percentage of surveyed transcripts containing frameshift mutations (column E). Total loss of function edits (Column G), was calculated by adding percentage of transcripts lost to nonsense mediated decay with percentage out-of-frame edits in the total pool of transcripts (Column B+Column F).

FIG. 11 depicts calculations of percentage in-frame edited transcripts in human DRG explants treated with AAV5-CMV-Cas9-guide at 3×10¹¹VG/ml. Percentage lost to nonsense mediated decay (column B) was determined by RT-ddPCR, as described in FIG. 9. Percentage indels created (Column C) was determined by drop-off RT-ddPCR, as described in FIG. 7. Percentage of indels which produce an in-frame mutation (Column D) was determined by targeted RNA-sequencing, as shown in FIG. 8. The percentage of transcripts in the sample which contain an in-frame mutation (Column E) was calculated by multiplying columns C and D (percentage transcripts with indels*percentage of indel transcripts with in-frame mutation). Because the pool of transcripts analyzed by RT-ddPCR and targeted RNA-sequencing methods does not include transcripts which were degraded due to nonsense mediated decay, the percentage of transcripts containing in-frame mutations as a proportion of total pool of transcripts, including both transcripts which are still present and those lost to nonsense mediated decay, was calculated (Column F), by multiplying the percentage of the total pool of RNA which has not been degraded (100%-Column B) by percentage of surveyed transcripts containing in-frame mutations (column E).

### DEFINITIONS

Throughout the specification, several terms are employed that are defined in the following paragraphs. Other definitions are also found within the body of the specification.

As used herein, the terms “about” and “approximately,” in reference to a number, is used herein to include numbers that fall within a range of 20%, 10%, 5%, or 1% in either direction (greater than or less than) of the number unless otherwise stated or otherwise evident from the context (except where such number would exceed 100% of a possible value).

As used herein, the term “cleavage” refers to the breakage of the covalent backbone of a DNA molecule. In some embodiments, cleavage can be initiated by a variety of methods including, but not limited to, enzymatic or chemical hydrolysis of a phosphodiester bond. In some embodiments, both single-stranded cleavage and double-stranded cleavage are possible, and double-stranded cleavage can occur as a result of two distinct single-stranded cleavage events. In some embodiments, DNA cleavage can result in the production of either blunt ends or cohesive ends.

As used herein, a “conservative substitution” refers to a substitution of an amino acid made among amino acids within the following groups: i) methionine, isoleucine, leucine, valine, ii) phenylalanine, tyrosine, tryptophan, iii) lysine, arginine, histidine, iv) alanine, glycine, v) serine, threonine, vi) glutamine, asparagine and vii) glutamic acid, aspartic acid. In some embodiments, a conservative amino acid substitution refers to an amino acid substitution that does not alter the relative charge or size characteristics of the protein in which the amino acid substitution was made.

As used herein, the term “heterologous,” in reference to polypeptide domains, refers to the fact that the polypeptide domains do not naturally occur together (e.g., in the same polypeptide). For example, in fusion proteins generated by the hand of man, a polypeptide domain from one polypeptide may be fused to a polypeptide domain from a different polypeptide. The two polypeptide domains would be considered “heterologous” with respect to each other, as they do not naturally occur together.

As used herein, the term “host cell” is a cell that is manipulated according to the present disclosure, e.g., into which nucleic acids are introduced. A “transformed host cell” is a cell that has undergone transformation such that it has taken up exogenous material such as exogenous genetic material, e.g., exogenous nucleic acids.

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. The comparison of sequences and determination of percent identity between two sequences can be accomplished using a mathematical algorithm. As is well known in the art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999.

As used herein, the term “operably linked” refers to a juxtaposition wherein the components described are in a relationship permitting them to function in their intended manner. In some embodiments, a regulatory element is “operably linked” to a functional element. In some such embodiments, an operably linked regulatory element is associated in such a way that expression and/or activity of the functional element is achieved under conditions compatible with the regulatory element. In some embodiments, “operably linked” regulatory elements are contiguous (e.g., covalently linked) with the coding elements of interest; in some embodiments, regulatory elements act in trans to or otherwise at a from the functional element of interest.

As used herein, the term “nuclease” refers to a polypeptide capable of cleaving the phosphodiester bonds between the nucleotide subunits of nucleic acids; the term “endonuclease” refers to a polypeptide capable of cleaving the phosphodiester bond within a polynucleotide chain.

As used herein, the terms “nucleic acid”, “nucleic acid molecule” or “polynucleotide” are used herein interchangeably. They refer to a polymer of deoxyribonucleotides or ribonucleotides in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “oligonucleotide” refers to a string of nucleotides or analogues thereof. In some embodiments, oligonucleotides may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on the intended function or use of the oligonucleotide. Throughout the specification, whenever an oligonucleotide is represented by a sequence of letters (chosen from the four base letters: A, C, G, and T, which denote adenosine, cytidine, guanosine, and thymidine, respectively), the nucleotides are presented in the 5′ to 3′ order from the left to the right. In certain embodiments, the sequence of an oligonucleotide includes one or more degenerate residues described herein.

As used herein, the term “off-target” refers to binding, cleavage and/or editing of an unintended or unexpected region of DNA by an RNA guided nuclease. In some embodiments, a region of DNA is an off-target region when it differs from the region of DNA intended or expected to be bound, cleaved and/or edited by 1, 2, 3, 4, 5, 6, 7 or more nucleotides.

As used herein, the term “on-target” refers to binding, cleavage and/or editing of an intended or expected region of DNA by an RNA guided nuclease.

As used herein, the term “polynucleotide” generally has its art-recognized meaning of a polymer of nucleic acids. The term is also used to refer to specific functional classes of polynucleotides, such as, for example, guide RNA molecules.

As used herein, the term “polypeptide” generally has its art-recognized meaning of a polymer of amino acids. The term is also used to refer to specific functional classes of polypeptides such as for example nucleases antibodies etc

As used herein, the term “regulatory element” refers to a DNA sequence that controls or impacts one or more aspects of gene expression. In some embodiments, a regulatory element is or includes a promoter, an enhancer, a silencer, and/or a termination signal. In some embodiments, a regulatory element controls or impacts inducible expression.

As used herein, the term “target site” refers to a portion of a double-stranded nucleic acid to which a binding molecule, e.g., a guide RNA or a guide RNA:Cas complex, will bind, provided sufficient conditions for binding exist. Typically, the target site comprises a nucleic acid sequence to which a binding molecule, e.g., a guide RNA or a guide RNA:Cas complex described herein, binds and/or that is cleaved as a result of such binding. In some embodiments, a target site comprises a nucleic acid sequence (also referred to herein as a target sequence or protospacer) that is complementary to a DNA sequence to which the targeting sequence (also referred to herein as the spacer) of a guide RNA described herein binds. In the context of RNA-guided nucleases, e.g., CRISPR/Cas nucleases, a target site typically comprises a nucleotide sequence (also referred to herein as a target sequence or a protospacer) that is complementary to a sequence comprised in a guide RNA (also referred to herein as the targeting sequence or the spacer) of the RNA-programmable nuclease. The target site further comprises a protospacer adjacent motif (PAM) at the 3′ end or 5′ end adjacent to the guide RNA-complementary sequence. For the RNA-guided nuclease Cas9, the target sequence may be, in some embodiments, 16-24 base pairs plus a 3-6 base pair PAM (e.g., NNN, wherein N represents any nucleotide). Exemplary PAM sequences for RNA-guided nucleases, such as Cas9, are known to those of skill in the art and include, without limitation, NNG, NGN, NAG, NGA, NGG, NGAG and NGCG wherein N represents any nucleotide. In addition, Cas9 nucleases from different species have been described, e.g., S. thermophilus recognizes a PAM that comprises the sequence NGGNG, and Cas9 from S. aureus recognizes a PAM that comprises the sequence NNGRRT. Additional PAM sequences are known in the art, including, but not limited to NNAGAAW and NAAR (see, e.g., Esvelt and Wang, Molecular Systems Biology, 9:641 (2013), the entire contents of which are incorporated herein by reference). For example, the target site of an RNA-guided nuclease, such as, e.g., Cas9, may comprise the structure [Nz]-[PAM], where each N is, independently, any nucleotide, and z is an integer between 1 and 50. In some embodiments, z is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, Z is 20.

### DETAILED DESCRIPTION

The present disclosure encompasses, in part, the discovery of RNA-guided nucleases that can target one or more DNA sequences in order to modify the one or more sequences in a way that impacts one or more symptoms of a pain disorder and/or a disorder associated with pain in a subject. Accordingly, in some embodiments, the present disclosure provides one or more technologies (e.g., compositions, systems, methods, etc.) related to genomic editing, e.g., a gene of interest. In some embodiments, a gene of interest is SCN9A and/or SCN10A. In some embodiments, a protein of interest is NaV1.7 and/or NaV1.8.

### Gene Editing Systems

In some embodiments, the present disclosure provides technologies for delivery of one or more functional effector proteins, e.g., nucleases, e.g., site-specific nucleases, e.g. Cas9 nucleases (including variants and fusions thereof), and/or nucleic acids (e.g., guide RNAs (gRNAs)) to a cell (e.g., in-vitro, ex-vivo, in-vivo). In some embodiments, the gene editing proteins include CRISPR/Cas proteins.

In some embodiments, technologies provided by the present disclosure may be useful for delivery of such functional effector proteins into a cell, e.g., in the context of manipulating the cell for a research or therapeutic purpose, e.g., via gene editing. For example, delivery of site-specific proteins, such as Cas proteins (or variants or fusions thereof) using the technologies provided herein allows for the targeted manipulation/modification of the genome of a host cell, e.g., in or around SCN9A and/or SCN10A, in-vitro, ex-vivo, or in-vivo.

In some embodiments, gene editing refers to adding, disrupting and/or changing a sequence of specific genes and/or regulatory elements thereof by insertion, removal and/or substitution or mutation of DNA from a genome using artificially engineered proteins and related molecules. For example, gene editing proteins, such as Cas nucleases, may be delivered to a cell using a method described herein. The gene editing protein can introduce double stranded breaks at a target locus in the host cell genome, resulting in altered gene function and/or expression. The activity of gene editing proteins can also promote DNA repair (e.g., non-homologous end joining or homology-directed repair), which is useful for rescue construct-mediated stable integration of foreign genetic material into the genome of a host cell.

In some embodiments, a CRISPR/Cas9-based gene editing approach can be used. A CRISPR (clustered regularly interspaced short palindromic repeats)/Cas9 gene editing system can efficiently disrupt genes at desired loci, enabling either complete gene knockout or homology directed repair. For example, in some embodiments, a guide RNA (gRNA) directs a site-directed polypeptide (e.g., an RNA-guided nuclease, e.g., a Cas9 endonuclease) to specific sites in the genome proximal to a protospacer adjacent motif (PAM), causing a double-strand break (DSB). Host cell machinery efficiently repairs the break via a non-homologous end joining (NHEJ) pathway, and during this process also introduces one or more changes (e.g., insertions or deletions (“indels”)) at the target site. Accordingly, gene disruption (e.g., frameshifts) can be achieved if the one or more changes (e.g., indels) in a target site is/are within the coding sequence of a given gene.

In some embodiments, a site-directed polypeptide is or comprises a Casendonuclease. In some embodiments, a Cas endonuclease is or comprises, for example, S. pyogenes Cas9, S. aureus Cas9, N meningitides Cas9, S. thermophilus CRISPRI Cas9, S. thermophilus CRISPR3 Cas9, T denticola Cas9, and Acidaminococcus sp. BV3L6 Cpf1, and variants thereof. Additional suitable Cas nucleases are known in the art. In some preferred embodiments, an endonuclease is a Cas9 endonuclease. In some such embodiments, a Cas9 endonuclease is an S. aureus endonuclease.

In some embodiments, a genome editing system uses a guide RNA (gRNA) comprising a spacer that comprises or consists of the nucleotide sequence of any one of SEQ ID Nos. 1-310. In some embodiments, a gRNA targets a target sequence. In some such embodiments, a target sequence is a gene or regulatory region of SCN9A and/or SCN10A.

In some embodiments, a gRNA that targets SCN9A comprises a spacer that comprises or consists of the nucleotide sequence of any one of SEQ ID Nos. 1-176.

In some embodiments, a gRNA that targets SCN10A comprises a spacer that comprises or consists of the nucleotide sequence of any one of SEQ ID Nos. 177-310.

In some embodiments, a system comprises at least one gRNA of SEQ ID Nos. 1-310 and a site-directed polypeptide (e.g., an RNA-guided nuclease described herein).

In some embodiments, a gRNA is or comprises an sgRNA (single-guide RNA), which sgRNA combines tracrRNA and crRNA (the two, separate molecules in the native CRISPR/Cas9 system in S. pyogenes) into a single RNA construct. In some such embodiments, such an sgRNA may be used for plasmid or in-vitro translation (IVT) expression.

In some embodiments, a gene editing system of the disclosure can be adapted for treating one or more symptoms of a pain disorder and/or disorder associated with pain, which one or more symptoms result from dysfunction/dysregulation in/of a voltage gated sodium channel, e.g., NaV1.7 and/or NaV1.8, or its subunits (e.g., SCN9A and/or SCN10A). For example, CRISPR/Cas9 components for targeting a gene encoding a subunit of NaV1.7 (i.e., SCN9A) and/or NaV1.8 (i.e., SCN10A) can be delivered to a subject (e.g., a human subject), having one or more mutant alleles, for example a dominant allele, of a gene encoding a subunit of NaV1.7 (i.e., SCN9A) and/or NaV1.8 (i.e., SCN10A).

SCN9A has a cytogenetic location of 2q24.3 and the genomic coordinate are on Chromosome 2 on the forward strand at position 166, 195, 185-166,375,993. A nucleotide sequence of SCN9A is shown as SEQ ID NO: 311. SCN7A is the gene upstream of SCN9A on the reverse strand and RN7SKP152 is the gene downstream of SCN9A on the reverse strand. AC010127.3 is a gene located on the forward strand opposite of SCN9A. SCN9A has a NCBI gene ID of 6335, Uniprot ID of Q15858 and Ensembl Gene ID of ENSG00000169432. SCN9A has 3906 SNPs, 39 introns and 38 exons. Sequences for the SCN9A gene, and SNPs, among other things, are provided for in International Publication No. WO/2018/007980.

In some embodiments, SCN9A may also be referred to as Sodium Channel, Voltage Gated, Type IX Alpha Subunit, Sodium Voltage-Gated Channel Alpha Subunit 9, Sodium Channel, Voltage-Gated, Type IX, Alpha Polypeptide, Voltage-Gated Sodium Channel Subunit Alpha Navl. 7, Sodium Channel Protein Type IX Subunit Alpha, Neuroendocrine Sodium Channel, Peripheral Sodium Channel 1, HNE-Na, NENA, PN1, GEFSP7, HSAN2D, Navl 0.7, FEB3B, ETHA, and SFNP.

SCN10A has a cytogenetic location of 3p22.2 and the genomic coordinate are on Chromosome 3 on the reserve strand at position 38,696,802-38,794,010. A nucleotide sequence of human SCN10A is shown as SEQ ID NO: 312. AC116038.1 is the gene upstream of SCN10A on the reverse strand and SCN5A is the gene downstream of SCN10A on the reverse strand. SCN10A has a NCBI gene ID of 6336, Uniprot ID of Q9Y5Y9 and Ensembl Gene ID of ENSG00000185313. SCNIOA has 6380 SNPs, 26 introns and 27 exons. Sequences for the SCN10A gene, and SNPs, among other things, are provided for in International Publication No. WO/2018/007976.

In some embodiments, SCN10A may also be referred to Sodium Channel, Voltage Gated, Type X Alpha Subunit, Sodium Voltage-Gated Channel Alpha Subunit 10, Sodium Channel Voltage-Gated Type X Alpha Polypeptide, Voltage-Gated Sodium Channel Subunit Alpha Nav1.8, Sodium Channel Protein Type X Subunit Alpha, Peripheral Nerve Sodium Channel 3, HPN3, PN3, Navl 0.8, FEPS2, and SNS.

In some embodiments, a gene editing system of the disclosure can be used to modify one or more wild-type or non-wild-type alleles at one or more loci of a targeted gene (e.g., SCN9A and/or SCN10A). In some embodiments, a replacement copy of a gene encoding a non-wild-type alpha subunit of NaV1.7 and/or NaV1.8 protein can be provided. In some embodiments, a replacement copy of a gene is not provided. In some such embodiments, a targeted gene may be modified in a way that produces a change such that protein is no longer produced from that gene (e.g., a frameshift mutation that results in no functional protein produced from the gene).

### Genome Engineering Strategies

In some embodiments, technologies (e.g., methods) of the present disclosure can involve editing one or both alleles of a particular gene. Gene editing to modify the allele(s) has the advantage of permanently altering the target gene or gene products in a particular cell or cells derived therefrom. In some embodiments, any CRISPR endonuclease may be used in the methods of the present disclosure. As is known to those of skill in the art, any given CRISPR endonuclease has its own associated PAM, which may or may not be disease, condition, and/or disorder specific. For example, in some embodiments,

In some embodiments, methods of the present disclosure are performed in-vitro, ex-vivo, and/or in-vivo.

In some embodiments, ex-vivo methods of the present disclosure can comprise editing cells of or derived from a peripheral nervous system (e.g., a neuron or a glial cell such as Schwann cell in nerves or satellite glial cell in ganglia, a neuronal progenitor, or neural precursor cell). In some such embodiments, one or more cells is isolated from a subject (e.g., patient in need of treatment, a control subject, etc.) using one or more aspects of gene editing as described herein. In some embodiments, ex-vivo methods in accordance with the present disclosure can comprise editing a patient-specific cell (e.g., iPSC or mesenchymal stem cell, neural stem cell, neuronal progenitor cell, neuronal cell, etc.).

In some embodiments, methods of the present disclosure are performed in-vitro. In some such embodiments, in-vitro methods of the present disclosure can comprise editing cells in culture, wherein such cells have been obtained from, e.g., a cell line and have been or will be differentiated into a committed progenitor cell, e.g., a neuronal progenitor cell.

In some embodiments, methods of the present disclosure are performed in-vivo. In some such embodiments, in-vivo methods of the present disclosure can comprise editing cells in patients with or at risk of one or more symptoms of a pain disorder and/or a disorder associated with pain using one or more aspects of gene editing as described herein.

In some embodiments, whether ex-vivo, in-vitro, or in-vivo, the present disclosure provides technologies that comprise editing the SCN9A and/or SCN10A gene(s) in a human cell using methods and approaches comprising gene editing as described herein.

In some embodiments, patients experiencing or at risk of one or more symptoms of a pain disorder or a disorder associated with pain may exhibit a wide range of changes (e.g., insertions, deletions, substitutions, etc.) in the SCN9A and/or SCN10A gene(s) relative to wild type SCN9A and/or SCN10A gene(s). In some embodiments, patients experiencing or at risk of one or more symptoms of a pain disorder or a disorder associated with pain may exhibit no of changes (e.g., insertions, deletions, substitutions, etc.) in the SCN9A and/or SCN10A gene(s). Accordingly, in some embodiments, different patients may require different editing strategies.

For example, in some embodiments, expression of the SCN9A and/or SCN10A gene(s) may be disrupted or eliminated by introducing random insertions or deletions (indels) that arise due to the imprecise NHEJ repair pathway. In some embodiments, target regions may be the coding sequences of the SCN9A and/or SCN10A gene(s) (i.e., exons). In some embodiments, inserting or deleting nucleotides into the coding sequence of a gene may cause a “frame shift” where the normal 3-letter codon pattern is disturbed. In some such embodiments, gene expression and therefore protein production can be reduced or eliminated. Similar approaches may also be used to target any intron, intron:exon junction, or regulatory DNA element of the SCN9A and/or SCN10A gene(s) where sequence alteration may interfere with the expression of the SCN9A and/or SCN10A gene(s) and any resulting gene product(s).

In some embodiments, NHEJ can also be used to delete segments of a target gene (e.g., SCN9A and/or SCN10A), either directly or by altering splice donor or acceptor sites through cleavage by one gRNA targeting several locations, or several gRNAs. In some embodiments, such an approach can be useful if small random indels are inefficient to knock-out the target gene or genes. In some embodiments, pairs of gRNAs (e.g., targeting different locations within a gene or regulatory element and/or targeting different genes) have been used for this type of deletions. Without a donor present, the ends from a DNA break or ends from different breaks can be joined using the several non-homologous repair pathways in which the DNA ends are joined with little or no base-pairing at the junction. In addition to canonical NHEJ, there are similar repair mechanisms, such as alt-NHEJ. If there are two breaks, the intervening segment can be deleted or inverted. In some embodiments, NHEJ repair pathways can lead to insertions, deletions or mutations at the joints.

In some embodiments, NHEJ can also lead to homology-independent target integration. For example, inclusion of a nuclease target site on a donor plasmid can promote integration of a transgene into the chromosomal double-strand break following in-vivo nuclease cleavage of both the donor and the chromosome (Cristea, Biotechnol Bioeng. 2013 March; 1 10(3):871-80). NHEJ was used to insert a 15-kb inducible gene expression cassette into a defined locus in human cell lines after nuclease cleavage. (See e.g., Maresca, M., Lin, V. G., Guo, N. & Yang, Y., Genome Res 23, 539-546 (2013); Suzuki et al. Nature, 540, 144-149 (2016)). In some such embodiments, the integrated sequence may disrupt the reading frame of the SCN9A and/or SCN10A gene(s) or alter the structure (e.g., primary structure, secondary structure, etc.) of the gene(s).

In some embodiments, homology directed repair (HDR) can also be used to knock-out a gene or alter the gene function. For example, an HDR knock-out strategy can comprise disrupting the structure or function of the SCN9A and/or SCN10A gene(s) by inserting into the SCN9A and/or SCN10A gene(s) a nonfunctional or irrelevant sequence. In some such embodiments, this can be achieved by inducing one single stranded break or double stranded break in the gene of interest with one or more CRISPR endonucleases and a gRNA {e.g., crRNA +tracrRNA, or sgRNA), or two or more single stranded breaks or double stranded breaks in the gene of interest with one or more CRISPR endonucleases and two or more gRNAs, in the presence of a donor DNA template introduced exogenously to direct the cellular DSB response to Homology-Directed Repair (the donor DNA template can be a short single stranded oligonucleotide, a short double stranded oligonucleotide, a long single or double stranded DNA molecule). In some instances, this approach can require development and optimization of gRNAs and donor DNA molecules for the SCN9A and/or SCN10A gene(s).

Generally, homology directed repair (HDR) is considered an error-free mechanism that uses a supplied homologous DNA sequence as a template during DSB repair. The rate of HDR is a function of the distance between the mutation and the cut site so choosing overlapping or nearest target sites is important. Templates can include extra sequences flanked by the homologous regions or can contain a sequence that differs from the genomic sequence, thus allowing sequence editing. The most common form of HDR is homologous recombination. There are additional pathways for HDR, including single-strand annealing and alternative-HDR. Genome engineering tools allow researchers to manipulate the cellular homologous recombination pathways to create site-specific modifications to the genome.

In some embodiments, cells can repair a double-stranded break using a synthetic donor molecule provided in trans. Therefore, in some embodiments, by introducing a double-stranded break near a specific mutation and providing a suitable donor, targeted changes can be made in the genome. In some embodiments, specific cleavage increases the rate of HDR more than 1,000 fold above the rate of 1 in 10<6>cells receiving a homologous donor alone. The rate of homology directed repair (HDR) at a particular nucleotide is a function of the distance to the cut site, so choosing overlapping or nearest target sites is important. Accordingly, in some embodiments, the present disclosure appreciates that technologies comprising gene editing offer advantages over, e.g., gene addition, as, in some embodiments, editing in situ can leave the rest of the genome essentially unperturbed.

In some embodiments, supplied donors for editing by HDR vary markedly but can contain a particular portion of a target sequence with small or large flanking homology arms to allow annealing to the genomic DNA. In some embodiments, homology regions flanking the introduced genetic changes can be 30 bp or smaller, or as large as a multi-kilobase cassette that can contain promoters, cDNAs, etc. In some embodiments, single-stranded and/or double-stranded oligonucleotide donors can be used. In some such embodiments, oligonucleotides range in size from less than 100 nt to over many kb, though longer ssDNA can also be generated and used. In embodiments comprising double-stranded donors, such donors may be or include, without limitation, PCR amplicons, plasmids, and mini-circles.

In some embodiments, an AAV vector can be a very effective means of delivery of a donor template, though the packaging limits for individual donors is <5kb. In some embodiments, active transcription of a donor can increase HDR three-fold, indicating the inclusion of promoter may increase conversion. In some embodiments, CpG methylation of a donor can decrease gene expression and HDR.

In addition to wild-type endonucleases, such as Cas9, nickase variants exist that have one or the other nuclease domain inactivated resulting in cutting of only one DNA strand. HDR can be directed from individual Cas nickases or using pairs of nickases that flank the target area. Donors can be single-stranded, nicked, or dsDNA.

In some embodiments, donor DNA can be supplied with a nuclease or independently by a variety of different methods, for example by transfection, nano-particle, micro-injection, or viral transduction. A range of tethering options has been proposed to increase the availability of the donors for HDR. For example, in some embodiments, tethering is or comprises attaching the donor to the nuclease, attaching to DNA binding proteins that bind nearby, or attaching to proteins that are involved in DNA end binding or repair.

One of skill in the art will appreciate that choice of repair pathway can be guided by a number of culture conditions, such as those that influence cell cycling, or by targeting of DNA repair and associated proteins. For example, in some embodiments, key NHEJ molecules (e.g., KU70, KU80 or DNA ligase IV) can be suppressed to, e.g., increase HDR.

In addition to gene editing by NHEJ or HDR, in some embodiments, site-specific gene insertions that use both the NHEJ pathway and HDR may be used. In some embodiments, a combination approach may be applicable in certain settings. In some such embodiments, such an approach may include intron/exon borders. In some embodiments, NHEJ may prove effective for ligation in the intron, while the error-free HDR may be better suited in the coding region.

The SCN9A and/or SCN10A gene(s) contain(s) a number of exons which are known in the art. Any one or more of these exons or nearby introns can be targeted in order to create one or more changes (e.g., indels) that modify (e.g., disrupt) the reading frame and eventually eliminate the SCN9A and/or SCN10A protein presence and/or function/activity.

In some embodiments, technologies of the present disclosure provide gRNA pairs that make a deletion by cutting the gene twice at locations flanking an unwanted sequence. This sequence may include one or more exons, introns, intron:exon junctions, other DNA sequences encoding regulatory elements of the SCN9A and/or SCN10A gene(s) or combinations thereof. The cutting can be accomplished by a pair of DNA endonucleases that each makes a DSB in the genome, or by multiple nickases that together make a DSB in the genome.

In some embodiments, the present disclosure can provide one gRNA to make one double-strand cut within a coding or splicing sequence. The double-strand cut can be made by a single DNA endonuclease or multiple nickases that together make a DSB in the genome.

In some embodiments, splicing donor and acceptors are generally within 100 base pairs of the neighboring intron. In some such embodiments, the present disclosure provides technologies that can provide gRNAs that cut approximately +/−100-3100 bp with respect to each exon/intron junction of interest.

Any of the gene editing strategies described or derived from the present disclosure, gene editing can be confirmed by sequencing or PCR analysis.

### Target Sequence Selection

In some embodiments, shifts in the location of the 5′ boundary and/or the 3′ boundary relative to particular reference loci can be used to facilitate or enhance particular applications of gene editing, which depend in part on the endonuclease system selected for the editing, as further described herein.

For example, many endonuclease systems have guidelines or criteria surrounding initial selection of potential target sites for cleavage, such as the requirement of a PAM sequence motif in a particular position adjacent to the DNA cleavage sites in the case of CRISPR Type II or Type V endonucleases.

In some embodiments, frequency of off-target activity for a particular combination of target sequence and gene editing endonuclease (i.e. the frequency of DSBs occurring at sites other than the selected target sequence) can be assessed relative to the frequency of on-target activity. In some embodiments, cells that have been correctly edited at a desired locus can have a selective advantage relative to other cells. By way of non-limiting example, a selective advantage may be or comprise acquisition of attributes such as enhanced rates of replication, persistence, resistance to certain conditions, enhanced rates of successful engraftment or persistence in-vivo following introduction into a patient, and other attributes associated with the maintenance or increased numbers or viability of such cells.

In some embodiments, cells that have been correctly edited at a desired locus can be positively selected for by one or more screening methods used to identify, sort or otherwise select for cells that have been correctly edited. Both selective advantage and directed selection methods can take advantage of the phenotype associated with the alteration.

In some embodiments, cells can be edited two or more times in order to create a second modification that creates a new phenotype that is used to select or purify the intended population of cells. In some such embodiments, a second modification could be created by adding an additional (e.g., second, third, fourth) gRNA enabling expression of a selectable or screenable marker. In some embodiments, cells can be correctly edited at a desired locus using a DNA fragment that contains the cDNA and also a selectable marker.

In some embodiments, target sequence selection can be guided by consideration of off-target frequencies in order to enhance the effectiveness of a given gene editing application and/or reduce the potential for undesired alterations at sites other than the desired target. In some embodiments, occurrence of off-target activity can be influenced by a number of factors including similarities and dissimilarities between the target site and various off-target sites, as well as the particular endonuclease used. Bioinformatics tools are available that assist in prediction of off-target activity, and frequently such tools can also be used to identify the most likely sites of off-target activity. In some such embodiments, assessments of off-target activity can then be confirmed or analyzed in experimental settings to evaluate relative frequencies of off-target to on-target activity. In some such embodiments, selection of sequences that have higher relative on-target activities can be achieved.

In some embodiments, target sequence selection can relate to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). In some embodiments, many such inducers cause DSBs to occur indiscriminately in the genome, and DSBs can be regularly induced and repaired in normal cells. During repair, the original sequence can often be reconstructed with complete fidelity, however, in some embodiments, small insertions or deletions (referred to as “indels”) are introduced at the DSB site.

In some embodiments, DSBs can be specifically induced at particular locations, as in the case of certain endonucleases systems described herein. In some such embodiments, such systems can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a donor polynucleotide, into a desired chromosomal location.

In some embodiments, regions of homology between particular sequences, which can be or comprise small regions of microhomology (comprising ten base pairs or fewer), can also be used to bring about desired deletions. For example, in some embodiments, a single DSB can be introduced at a site that exhibits microhomology with a nearby sequence. In some such embodiments, during the normal course of repair of such DSB, a result that occurs with high frequency is the deletion of the intervening sequence as a result of recombination being facilitated by the DSB and concomitant cellular repair process.

In some embodiments, selecting target sequences within regions of homology can also give rise to much larger deletions, including gene fusions (when deletions are in coding regions), which may or may not be desired given the particular circumstances.

The present disclosure provides and describes herein methods of selection of various target regions for the creation of DSBs designed to induce insertions, deletions or mutations that result in reduction or elimination of SCN9A and/or SCN10A gene(s) and resultant gene products (e.g., proteins) and activity, as well as the selection of specific target sequences within such regions that are designed to minimize off-target events relative to on-target events.

### Vectors

In some embodiments, one or more components of a gene editing system described herein is expressed using an expression vector. In some embodiments, one or more components of a gene editing system described herein is administered to a subject using a viral vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into a host cell, and thereby are replicated along with the host genome. In some embodiments, vectors can be capable of directing expression of nucleic acids to which they are operatively linked (e.g., often referred to as recombinant expression vectors or expression vectors).

In some embodiments, a regulatory sequence includes, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, CA (1990). Regulatory sequences can include sequences that direct constitutive expression of a nucleotide sequence in many types of host cells, as well as those that direct expression of the nucleotide sequence only in certain host cells (e.g., tissue-specific regulatory sequences). It will be appreciated by those skilled in the art that the design of the expression vector can depend factors such as choice of host cell, level of expression desired or required, etc.

In some embodiments, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, a vector can be a self-inactivating vector that either inactivates the viral sequences or the components of the CRISPR machinery or other elements. In some embodiments, a vector includes a promoter. In some embodiments, suitable eukaryotic promoters (i.e., promoters functional in a eukaryotic cell) include but are not limited to those from cytomegalovirus (CMV) immediate early, minimal CMV, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphogly cerate kinase-1 locus promoter (PGK), and mouse metallothionein-I.

In some embodiments, a promoter can be a neuron specific promoter. Neuron specific promoters are known in the art and include, e.g., those derived from synapsin I (SYN), calcium/calmodulin-dependent protein kinase II Beta (CAMK2B), calcium/calmodulin-dependent protein kinase II alpha (CAMK2A), Methyl-CpG Binding Protein 2 (MECP2), Somatostat, tubulin alpha I (TUBA1A), neuron-specific enolase (ENO2), eSYN (hybrid promoter consisting of a 0.4Kb CMV enhancer (E) and the 0.45Kb human Synapsin I promoter fragment) and/or platelet-derived growth factor beta (PDGFB) promoters. In some embodiments, alternative neuronal specific promoters as known in the art may be utilized in constructs described herein.

In some embodiments, a promoter can be a constitutive promoter (e.g., CMV promoter, UBC promoter). In some embodiments, a promoter can be an inducible promoter (e.g., a heat shock promoter, tetracycline-regulated promoter, steroid-regulated promoter, metal-regulated promoter, estrogen receptor-regulated promoter, etc.). In some embodiments, a promoter can be a spatially restricted and/or temporally restricted promoter (e.g., a tissue specific promoter, a cell-type-specific promoter, etc.).

For expressing small RNAs, including gRNAs (e.g., such as any of those of SEQ ID NOs: 1-310) as described herein and optionally used in connection with a site-directed polypeptide (e.g., an RNA-guided nuclease, e.g., a Cas endonuclease), various promoters such as RNA polymerase III promoters, (e.g., U6 and/or HI) may be suitable or, in some embodiments, can be advantageous. Descriptions of and parameters for selecting and/or enhancing use of such promoters are known in art (see, e.g., Ma, H. et al, Molecular Therapy—Nucleic Acids 3, el 61 (2014) doi: 10.1038/mtna.2014.12.

In some embodiments, an expression vector can also contain a ribosome binding site for translation initiation and/or a transcription terminator. In some embodiments, an expression vector can also comprise appropriate sequences for amplifying expression (e.g., of a given sequence contained therein). In some embodiments, an expression vector can include nucleotide sequences encoding non-native tags (e.g., histidine tag, hemagglutinin tag, green fluorescent protein, etc.). In some such embodiments, the non-native tag is fused to a site-directed polypeptide resulting in a fusion protein.

In some embodiments, a nucleic acid encoding a genome-targeting nucleic acid of the disclosure and/or a site-directed polypeptide can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. A variety of targeting moieties are known in the art and can be used to facilitate, improve and/or enhance interaction of such vehicles with desired cell types or locations.

In some embodiments, introduction of one or more complexes, polypeptides, and/or nucleic acids of the present disclosure into one or more cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, etc.

### Viral Vectors

By way of non-limiting example, in some embodiments, viral vectors or components thereof based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus), lentivirus, alphavirus, enterovirus, pestivirus, baculovirus, herpesvirus, Epstein Barr virus, papovavirus, poxvirus, and other recombinant vectors. In some embodiments a vector may be or comprise pXT1, pSG5, pSVK3, pBPV, pMSG, and pSVLSV40 (Pharmacia). In some preferred embodiments, one or more vectors comprises adeno-associated virus or components thereof. Other vectors can be used so long as they are compatible with the host cell to which a given vector is provided.

### AAV Vectors

In certain embodiments of the disclosure, a viral vector described herein is an adeno-associated viral (AAV) vector. AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012)). Methods for generating and using AAV vectors are described, for example, in U.S. Pat. Nos. 5,139,941 and 4,797,368.

Several AAV serotypes have been characterized, including AAV1, AAV2 (e.g., AAV2g9, AAV2.5, or AAV2i8), AAV3 (e.g., AAV3B), AAV4, AAV5, AAV6, AAV7, AAV8, AAV9, AAV10, and AAV 11, as well as variants and/or hybrids thereof. For example, in some embodiments, an AAV vector is an AAVrh10, AAVhu68, AAV2/5, AAV2/6, AAV2/8 or AAV2/9 vector (e.g., AAV6, AAV8 or AAV9 serotype having AAV2 ITR). Other AAV vectors are described in, e.g., Sharma et al., Brain Res Bull. 2010 Feb. 15; 81(2-3): 273. Generally, any AAV serotype may be used to deliver a transgene described herein. However, in some embodiments, serotypes have different tropisms, e.g., they preferentially infect different tissues, thus, in some embodiments, a serotype may be chosen based upon tropism for a particular tissue and/or cell type. In some embodiments, one or more AAV serotypes can be matched to one or more target cell types.

A recombinant adeno-associated virus (rAAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived, and may be from a different AAV serotype than the rAAV genome ITRs, including, but not limited to, AAV serotypes described herein. Production of pseudotyped rAAV is disclosed in, for example, international patent application publication number WO 01/83692.

The AAV sequences of an AAV vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. In some embodiments, substantially the entire sequences encoding the ITRs are used in an AAV vector, although some degree of minor modification of these sequences can be permissible. Ability to modify these ITR sequences is known to those of skill in the art. (See, e.g., texts such as Sambrook et al, “Molecular Cloning. A Laboratory Manual”, 2d ed., Cold Spring Harbor Laboratory, New York (1989); and K. Fisher et al., J Virol, 70:520 532 (1996)). For example, in some embodiments, an AAV vector of the present disclosure is a “cis-acting” plasmid containing a transgene, in which the selected transgene sequence and associated regulatory elements are flanked by the 5′ and 3′ AAV ITR sequences. The AAV ITR sequences may be obtained from any known AAV, including known mammalian AAV types and/or those described herein.

In some embodiments, an AAV vector may be a dual or triple AAV vector, e.g., for the delivery of large transgenes (e.g., transgenes of greater than approximately 5kb). In some embodiments, a dual AAV vector may include two separate AAV vectors, each including a fragment of the full sequence of the large transgene of interest, and when recombined, the fragments form the full sequence of the large transgene of interest, or a functional portion thereof.

In some embodiments, a triple AAV vector may include three separate AAV vectors, each including a fragment of the sequence of the large transgene of interest, and when recombined, the fragments form the full sequence of the large transgene of interest, or a functional portion thereof.

The multiple AAV vectors of the dual or triple AAV vectors can be delivered to and co-transduced into the same cell, where the two or three fragments of transgene recombine together and generate a single mRNA transcript of the entire large transgene of interest. In some embodiments, the fragmented transgenes include a non-overlapping sequences. In some embodiments, the fragmented transgenes include a specified overlapping sequences.

In some embodiments, the multiple AAV vectors of the dual or triple may be the same type of AAV vector (e.g., the same serotype, construct, etc.). In some embodiments, the multiple AAV vectors of the dual or triple may be different types of AAV vector (e.g., same serotype, construct, etc.).

Exemplary rAAV nucleic acid vectors useful in accordance with the present disclosure include single-stranded (ss) or self-complementary (sc) AAV nucleic acid vectors.

In some embodiments, an rAAV particle comprises a nucleic acid vector, such as a single-stranded (ss) or self-complementary (sc) AAV nucleic acid vector. In some embodiments, the nucleic acid vector contains an expression construct as described herein and one or more regions comprising inverted terminal repeat (ITR) sequences (e.g., wild-type ITR sequences or engineered ITR sequences) flanking the expression construct. In some embodiments, the nucleic acid is encapsidated by a viral capsid.

Accordingly, in some embodiments, a rAAV particle, vector and/or nucleic acid comprises a viral capsid and a nucleic acid vector as described herein, which is encapsidated by the viral capsid. In some embodiments, the viral capsid comprises 60 capsid protein subunits comprising VP1, VP2 and VP3. In some embodiments, the VP1, VP2, and VP3 subunits are present in the capsid at a ratio of approximately 1:1:10, respectively.

In some embodiments, ITR sequences of a nucleic acid or nucleic acid vector of the present disclosure can be derived from any AAV serotype (e.g., 1, 2, 3, 4, 5, 6, 7, 8, 9, or 10, or others as described herein) or can be derived from more than one serotype. In some embodiments, ITR sequences are derived from AAV2. In some embodiments, the ITR sequences are derived from one or more other serotypes. ITR sequences and plasmids containing ITR sequences are known in the art and are commercially available (see, e.g., products and services available from Vector Biolabs Philadelphia PA: Cellbiolabs San Diego CA: Agilent Technologies, Santa Clara, Ca; and Addgene, Cambridge, MA; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24): 14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313).

In some embodiments, an expression construct is at least 2, 3, 4, 5, 6, 7, 8, 9, 10 kilobases in size. In some embodiments, an expression construct is no more than 10, 9, 8, 7, 6, 5, 4, 3, 2, or less kilobases in size. In some embodiments, the expression construct is between 4 and 7 kilobases in size.

An rAAV vector, particle, and/or nucleic acid may be of any AAV serotype (e.g., 1, 2, 3, 4, 5, 6, 7, 8, 9, or 10), including any derivative (including non-naturally occurring variants of a serotype) or pseudotype.

In some embodiments, the rAAV serotype is rAAV2. In some embodiments, the rAAV serotype is rAAV5. In some embodiments, the rAAV serotype is rAAV8. In some embodiments, the rAAV serotype is rAAV9. In some embodiments, the rAAV serotype is rAAV5. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J.).

In some embodiments, an AAV vector may utilize or be based on a serotype selected from any of the following serotypes, and variants thereof including but not limited to AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5, AAV2i8, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAV A3.7, AAV-b, AAVCI, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu. 1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK 11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi. 1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8. In some embodiments, the AAV serotype may be, or have, a mutation in the AAV9 sequence as described by N Pulicherla et al. (Molecular Therapy 19(6): 1070-1078 (2011)), such as but not limited to, AAV9.9, AAV9.11, AAV9.13, AAV9.16, AAV9.24, AAV9.45, AAV9.47, AAV9.61, AAV9.68, AAV9.84.

In some embodiments, a serotype may be AAVDJ or a variant thereof, such as AAVDJ8 (or AAV-DJ8), as described by Grimm et al. (Journal of Virology 82(12): 5887-5911 (2008)). The amino acid sequence of AAVDJ8 may comprise two or more mutations in order to remove the heparin binding domain (HBD). By way of non-limiting example, an AAV-DJ sequence of SEQ ID NO: 1 in U.S. Pat. No. 7,588,772, may comprise two mutations: (1) R587Q where arginine (R; Arg) at amino acid 587 is changed to glutamine (Q; Gin) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gin) and (3) R590T where arginine (R; Arg) at amino acid 590 is changed to threonine (T; Thr).

In some embodiments, an AAV serotype may comprise or have a sequence as described in, e.g., U.S. Pat. No. 6,156,303, or derivatives thereof. In some embodiments, an AAV serotype may be or comprise a sequence as described in International Application Publication No. WO2015121501, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

In some embodiments, an AAV serotype may be from any number of species. For example, in some embodiments, an AAV may be an avian AAV (AAAV). In some embodiments, an rAAV serotype may be or comprise a sequence as described in U.S. Pat. No. 9,238,800, such as, but not limited to, AAAV (SEQ ID NO: 1, 2, 4, 6, 8, 10, 12, and 14 of U.S. Pat. No. 9,238,800), or variants thereof. In some embodiments, an AAV may be a bovine AAV (BAAV). The BAAV serotype may be or comprise a sequence as described in U.S. Pat. No. 9,193,769, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, such as, but not limited to, BAAV (SEQ ID NO: 5 and 6 of U.S. Pat. No. 7,427,396), or variants thereof. In some embodiments, an AAV may be a caprine AAV. The caprine AAV serotype may be or comprise a sequence as described in U.S. Pat. No. 7,427,396, such as, but not limited to, caprine AAV (SEQ ID NO: 3 of U.S. Pat. No. 7,427,396), or variants thereof.

In some embodiments, an AAV may be engineered as a hybrid AAV from two or more parental serotypes. In one example, the AAV may be AAV2G9 which comprises sequences from AAV2 and AAV9. The AAV2G9 AAV serotype may be, or have, a sequence as described in U.S. Patent Publication No. 20160017005. For example, in some embodiments, an AAV vector is an AAV2/5, AAV2/6, AAV2/8 or AAV2/9 vector (e.g., AAV6, AAV8 or AAV9 serotype having AAV2 ITR). Other AAV vectors are described in, e.g., Sharma et al., Brain Res Bull. 2010 Feb. 15; 81(2-3): 273.

In some embodiments, an AAV may be a serotype generated by the AAV9 capsid library with mutations in amino acids 390-627 (VP1 numbering) as described by Pulicherla et al. (Molecular Therapy 19(6): 1070-1078 (2011). In some such embodiments, a serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (CI 531 A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A,; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV may be a variant, such as PUP. A or PHP.B as described in Deverman. 2016. Nature Biotechnology. 34(2): 204-209.

In some embodiments, an rAAV vector, particle, and/or nucleic acid comprise(s) capsid proteins having one or more amino acid substitutions (e.g., AAV2(tripleY-F) (Y444F+Y500F+Y730F), see Petrs-Silva et al., Novel Properties of Tyrosine-mutant AAV2 Vectors in the Mouse Retina, Mol. Ther. 19(2): 293-301 (2011); AAV2(quadY-F+T-V) (Y272F+Y444F+Y500F+Y730F+T491V), see Kay et al., Targeting Photoreceptors via Intravitreal Delivery Using Novel, Capsid-Mutated AAV Vectors, PLoS One, 8(4): e62097 (2013); or AAV2(MAX)deltaHS, also known as AV2(4pMut)deltaHS (Y444F+Y500F+Y730F+T491V+R585S+R588T+R487G), see Boye et al., Impact of Heparan Sulfate Binding on Transduction of Retina by Recombinant Adeno-Associated Virus Vectors, J. Virol. 90(8): 4215-4231 (2016)).

In some embodiments, an rAAV vector, particle, and/or nucleic acid comprises a capsid that includes modified capsid proteins (e.g., capsid proteins comprising a modified VP3 region). Methods of producing modified capsid proteins are known in the art (see, e.g., US20130310443). In some embodiments, the rAAV vector, particle, and/or nucleic acid comprises a modified capsid protein comprising at least one non-native amino acid substitution at a position that corresponds to a surface-exposed amino acid (e.g., a surface exposed Tyrosine) in a wild-type capsid protein. In some embodiments, the rAAV vector, particle, and/or nucleic acid comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in the wild-type capsid protein, a non-lysine amino acid (e.g., a glutamic acid) at a position that corresponds to a surface-exposed lysine amino acid in the wild-type capsid protein, a non-serine amino acid (e.g., a valine) at a position that corresponds to a surface-exposed serine amino acid in the wild-type capsid protein, or a combination thereof.

In some embodiments, a rAAV vector, particle, and/or nucleic acid (e.g., a rAAV2 or other rAAV serotype particle) comprises a capsid that includes modified capsid proteins having at least 1, 2, 3, 4, 5, 6, 7, 8, 9, 10 or more amino acid substitutions.

In some embodiments, an expression construct including, for example, a promoter and a gene of interest, is flanked on each side by an inverted terminal repeat sequence. In some embodiments, the expression construct comprises one or more regions comprising a sequence that facilitates expression of the coding sequence of the gene of interest, e.g., expression control sequences operably linked to the coding sequence. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer). In some embodiments, the expression construct includes other regulatory elements such as, e.g., WPRE.

In some embodiments, a nucleic acid of the present disclosure is a plasmid (e.g., a circular nucleic acid comprising one or more of an origin of replication, a selectable marker, and a reporter gene). In some embodiments, a nucleic acid described herein, such as a plasmid, may also contain marker or reporter genes, e.g., LacZ or a fluorescent protein, and an origin of replication. In some embodiments, a plasmid is transfected into a producer cell that produces AAV particles comprising the expression construct.

In some embodiments, a nucleic acid of the present disclosure is a nucleic acid vector such as a recombinant adeno-associated virus (rAAV) genome.

In addition to the major elements identified above for an AAV vector, the vector can also include conventional control elements operably linked to the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters that are native, constitutive, inducible and/or tissue-specific, are known in the art and may be included in a vector described herein.

In some embodiments, one or more elements of one or more technologies as provided herein comprises one or more promoters.

For example, in some embodiments, the present disclosure provides a nucleic acid, which nucleic acid comprises an expression construct further comprising a promoter operably linked to a coding sequence of one or more genes of interest. In some embodiments, a promoter is a natural or naturally-derived promoter. In some embodiments a promoter can be a truncated natural promoter. In some embodiments, a promoter can include an enhancer and/or basal promoter elements from a natural promoter. In some embodiments, a promoter is a constitutive promoter as described herein. In some embodiments, a promoter is a cell- or tissue-specific promoter as described herein.

Examples of constitutive promoters include but are not limited to the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In another embodiment, a native promoter, or fragment thereof, for a transgene will be used. In a further embodiment, other native expression control elements, such as enhancer elements, polyadenylation sites or Kozak consensus sequences may also be used to mimic the native expression.

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken j-actin promoter, a Cbh promoter, a pol II promoter, or a pol III promoter.

In some embodiments, a promoter is a tissue or cell-specific promoter. For example, in some embodiments, neuronal-specific promoters may include, but are not limited to, human synapsin I (SYN) promoter (e.g., as described in Li et al., Proc Natl Acad Sci USA 1993; 90:1460-1464), mouse calcium/calmodulin-dependent protein kinase II (CaMKII) promoter (e.g., as described in Mayford et al., Proc Natl Acad Sci USA 1996; 93: 13250-13255), rat tubulin alpha I (Tal) promoter (e.g., as described in Gloster et al., J Neurosci 1994; 14: 7319-7330), rat neuron-specific enolase (NSE) promoter (e.g., as described in Forss-Petter et al, Neuron 1990; 5: 187-197), and human platelet-derived growth factor-beta chain (PDGF) promoter (e.g., as described in Sasahara et al, Cell 1991; 64: 217-227).

In some embodiments, a viral vector is designed for expressing a transgene described herein in neurons or neuronal progenitors, and viral vector (e.g., an AAV vector) includes one or more neuron-specific regulatory elements, which substantially limit expression of the transgene to neuronal cells or progenitors thereof. Generally, neuron-specific regulatory elements can be derived from any gene known to be exclusively expressed in neurons or neuronal progenitors of the central and/or peripheral nervous system. In some embodiments, a viral vector described herein includes a neuron-specific regulatory element derived from the genomic loci of one or more neuronal proteins.

### Host Cell

In some embodiments, the present disclosure provides transfected host cells. A number of transfection techniques are generally known in the art (see, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197). Such techniques can be used to introduce one or more exogenous nucleic acids, such as a nucleotide integration vector and other nucleic acid molecules, into suitable host cells.

In some embodiments, the present disclosure provides host cells that comprise one or more of rAAV particles, expression construct(s), or nucleic acid vector(s) as described herein. In some embodiments, host cells may be any cell type (e.g., mammalian, insect, etc.). In some embodiments, host cells are mammalian host cells. In some embodiments, host cells are non-human primate cells. In some preferred embodiments of the present disclosure, host cells are human host cells. In some embodiments, host cells are primary cells. In some embodiments, host cells are derived from a cell line (e.g., an immortalized cell line). In some embodiments, host cells may be isolated (e.g., using cell or tissue culture) or obtained via commercially available means that will be known to those of skill in the art. In some embodiments, host cells may be within a body of an animal (e.g., a non-human animal, e.g., a genetically modified mouse). In some embodiments, a host cell may be used as a recipient of an AAV helper construct, an AAV minigene plasmid, an accessory function vector, and/or other transfer DNA associated with the production of recombinant AAVs.

In some embodiments, a transgene or other genomic component of interest is flanked by ITRs and rep/cap genes are introduced into insect host cells by infection with baculovirus-based vectors. Such production systems are known in the art (see generally, e.g., Zhang et al., 2009, Human Gene Therapy 20:922-929). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. As will be appreciated by one of skill in the art, the foregoing methods for producing recombinant vectors are not meant to be limiting, and other suitable methods will be apparent to such individuals.

### Production

Methods for obtaining viral vectors are generally known in the art.

Methods of producing one or more rAAV vectors, particles and/or nucleic acid vectors are also known in the art and such vectors, particles, and/or nucleic acids or components thereof are commercially available (see, e.g., Zolotukhin et al. Production and purification of serotype 1, 2, and 5 recombinant adeno-associated viral vectors. Methods 28 (2002) 158 167; and U.S. Patent Publication Numbers US20070015238 and US20120322861; and plasmids and kits available from ATCC and Cell Biolabs, Inc.). General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al, Mol. Cell. Biol. 4:2072 (1984); Hermonat et al, Proc. Natl. Acad. Sci. USA, 81:6466 (1984); Tratschin et al, Mol. Cell. Biol. 5:3251 (1985); McLaughlin et al, J. Virol., 62: 1963 (1988); and Lebkowski et al, 1988 Mol. Cell. Biol., 7:349 (1988). Samulski et al (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13: 1244-1250; Paul et al. (1993) Human Gene Therapy 4:609-615; Clark et al. (1996) Gene Therapy 3: 1124-1132; U.S. Pat. Nos. 5,786,211; 5,871,982; and 6,258,595.

For example, to produce AAV vectors, methods typically involve culturing a host cell which contains a nucleic acid sequence encoding an AAV capsid protein or fragment thereof, a functional rep gene; a recombinant AAV vector composed of AAV inverted terminal repeats (ITRs) and a transgene; and/or sufficient helper functions to permit packaging of the recombinant AAV vector into the AAV capsid proteins. In some embodiments, a nucleic acid vector (e.g., as a plasmid) may be combined with one or more helper plasmids, e.g., that contain a rep gene (e.g., encoding Rep78, Rep68, Rep52 and Rep40) and a cap gene (encoding VP1, VP2, and VP3), and transfected into a producer cell line such that rAAV particles can be packaged and purified.

In some embodiments, components to be cultured in a host cell to package an AAV vector in an AAV capsid may be provided to the host cell in trans. In some embodiments, any one or more of the required components (e.g., recombinant AAV vector, rep sequences, cap sequences, and/or helper functions) may be provided by a stable host cell that has been engineered to contain one or more of the required components using methods known to those of skill in the art. In some embodiments, such a stable host cell contains the required component(s) under the control of an inducible promoter. In other embodiments, the required component(s) may be under the control of a constitutive promoter. In other embodiments, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. In some embodiments, stable host cells may be generated by one of skill in the art using routine methods.

In some embodiments, the one or more helper plasmids include a first helper plasmid comprising a rep gene and a cap gene and a second helper plasmid comprising other genes that assist in AAV production, such as a Ela gene, a Elb gene, a E4 gene, a E2a gene, and a VA gene. In some embodiments, the rep gene is a rep gene derived from AAV2 and the cap gene is derived from AAV5. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R.O. (2008), International efforts for recombinant adenoassociated viral vector reference standards, Molecular Therapy, Vol. 16, 1185-1188).

By way of non-limiting example, in some embodiments, a method of rAAV particle production comprises use of one or more helper plasmids, which helper plasmids comprise rep and cap ORFs for the desired AAV serotype and the adenoviral VA, E2A (DBP), and E4 genes under the transcriptional control of their native promoters. In some such embodiments, cells (e.g., HEK293 cells, which are widely commercially available, e.g., such as from ATCC®) are transfected via, e.g., CaP04-mediated transfection.

In some embodiments, an agent such as polyethylenimine (PEI) is used, in conjunction with the helper plasmid(s) and a plasmid containing a nucleic acid vector described herein. In some embodiments, recombinant AAV vector, rep sequences, cap sequences, and/or helper functions required for producing an AAV in accordance with the present disclosure may be delivered to a packaging host cell using any appropriate genetic element (e.g., vector). In some embodiments, a selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y.). Similarly, methods of generating AAV virions are well known and any suitable method may be used in accordance with the present disclosure (see, e.g., K. Fisher et al, J. Virol., 70:520-532 (1993) and U.S. Pat. No. 5,478,745).

In some embodiments, recombinant AAVs may be produced using a triple transfection method (e.g., as described in U.S. Pat. No. 6,001,650). In some embodiments, recombinant AAVs are produced by transfecting a host cell with a recombinant AAV vector (comprising a transgene) to be packaged into AAV particles, an AAV helper function vector, and an accessory function vector. In some embodiments, the AAV helper function vector encodes AAV helper function sequences (i.e., rep and cap), which function in trans for productive AAV replication and encapsidation. In some embodiments, the AAV helper function vector supports efficient AAV vector production without generating any detectable wild-type AAV virions (i.e., AAV virions containing functional rep and cap genes).

Non-limiting examples of vectors suitable for use with the present disclosure include, but are not limited to, pHLP19 (see, e.g., U.S. Pat. No. 6,001,650) and pRep6cap6 vector (see, e.g., U.S. Pat. No. 6,156,303). In some embodiments, an accessory function vector encodes nucleotide sequences for non-AAV derived viral and/or cellular functions upon which AAV is dependent for replication (i.e., “accessory functions”). Accessory functions include those functions required for AAV replication, including, without limitation, those moieties involved in activation of AAV gene transcription, stage specific AAV mRNA splicing, AAV DNA replication, synthesis of cap expression products, and AAV capsid assembly. In some embodiments, viral-based accessory functions can be derived from any known helper viruses such as adenovirus, herpesvirus (other than herpes simplex virus type-1), and vaccinia virus.

In some embodiments, the present disclosure provides, among other things, methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772. In some embodiments, a producer cell line is transiently transfected with a construct that encodes the transgene flanked by ITRs and a construct(s) that encodes rep and cap. In some embodiments, a packaging cell line that stably supplies rep and cap is transiently transfected with a construct encoding the transgene flanked by ITRs. In some embodiments, AAV virions are produced in response to infection with helper adenovirus or herpesvirus, and AAVs are separated from contaminating virus. In some embodiments, infection with helper virus is not required to recover the AAV. In some such embodiments, the helper functions (i.e., adenovirus E1, E2a, VA, and E4 or herpesvirus UL5, UL8, UL52, and UL29, and herpesvirus polymerase) are also supplied, in trans. In some embodiments, helper functions can be supplied by transient transfection of the cells with constructs that encode the helper functions, or the cells can be engineered to stably contain genes encoding the helper functions, the expression of which can be controlled at the transcriptional or posttranscriptional level.

For example, in some embodiments, Sf9-based producer stable cell lines are infected with a single recombinant baculovirus containing a nucleic acid vector of the present disclosure. In some embodiments, a mammalian cell line (e.g., HEK293 or BHK) is infected with an HSV containing a nucleic acid vector of the present disclosure and optionally one or more helper HSVs comprising rep and cap ORFs as described herein and adenoviral VA, E2A (DBP), and E4 genes under the transcriptional control of their native promoters. In some embodiments, infected cells (e.g., HEK293, BHK, or Sf9 cells) are then incubated for a time period (e.g., at least 20, 30, 40, 50, or 60 hours or more) to allow for sufficient rAAV particle production. In some such embodiments, the rAAV particles can then be purified using any method known in the art or described herein, e.g., by iodixanol step gradient, CsCl gradient, chromatography, or polyethylene glycol (PEG) precipitation.

### Cells for Genetic Modification

The present disclosure provides, among other things, compositions and methods of use thereof for ameliorating one or more signs, symptoms, and/or disorders associated with SCN9A and/or SCN10A gene(s), as described herein. In some embodiments, principal targets for gene editing are mammalian cells. In some embodiments, principal targets for gene editing are human cells. For example, in some embodiments, an ex-vivo method of gene editing may be used in conjunction with somatic cells, which, after being modified using technologies provided herein, can give rise to committed and/or differentiated cells (e.g., neuronal progenitor cells or neurons of the peripheral nervous system). In some embodiments, an in-vivo method of gene editing as described herein comprises human cells (e.g., human neurons of the peripheral nervous system) and/or affected organs.

In some embodiments, gene editing is performed on autologous cells. In some such embodiments, the present disclosure appreciates that by performing gene editing in autologous cells that are derived from and therefore already completely matched with the patient in need, it is possible to generate cells that can be safely re-introduced into the patient. In some such embodiments, such re-introduction of autologous cells may more effectively give rise to a population of cells that will be able to ameliorating or prevent one or more clinical conditions (e.g., one or more symptoms) associated with the patient's disease (e.g., a pain disorder and/or disorder associated with pain).

### Stem Cells

Stem cells are capable of both proliferation and giving rise to more progenitor cells, these in turn having the ability to generate a large number of mother cells that can in turn give rise to differentiated or differentiable daughter cells. In some embodiments, a stem cell may be, but does not have to be, multipotent (e.g., able to give rise to cells of different lineages, such as, e.g., skin cells, liver cells, bone cells, etc.). For example, a stem cell such as a neural stem cell may only give rise to cells of a neural lineage (e.g., neurons, glia, etc.). In some embodiments, a stem cell may be, e.g., an iPSC, a mesenchymal stem cell, a hematopoietic stem cell, a neural stem cell, etc. The daughter cells themselves can be induced to proliferate and produce progeny that subsequently differentiate into one or more mature cell types, while also retaining one or more cells with parental developmental potential. In some embodiments, a stem cell is a cell with the capacity or potential, under particular circumstances, to differentiate to a more specialized or differentiated phenotype (e.g., a progenitor cell), and which retains the capacity, under certain circumstances, to proliferate without substantially differentiating. As is known to one of skill in the art, cellular differentiation is a complex process typically occurring through many cell divisions. Thus, in some embodiments, a differentiated cell may derive from a multipotent cell that, itself, is derived from a multipotent cell, and so on. While each of these multipotent cells may be considered stem cells, the range of cell types that each can give rise to may vary considerably. In some embodiments, some differentiated cells also have the capacity to give rise to cells of greater developmental potential. In some embodiments, some differentiated cells are more restricted and may only give rise to cells of a particular lineage (e.g., neuronal lineage). Such capacity may be natural or may be induced artificially upon treatment with various factors.

In some embodiments, self-renewal can be another important aspect of the stem cell. In theory, self-renewal can occur by either of two major mechanisms. Stem cells can divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high-proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated,” or “differentiating” is a relative term. A “differentiated cell” is a cell that has progressed further down the developmental pathway than the cell to which it is being compared. Thus, stem cells can differentiate into lineage-restricted precursor cells (such as a myocyte progenitor cell), which in turn can differentiate into other types of precursor cells further down the pathway (such as a myocyte precursor), and then to an end-stage differentiated cell, such as a myocyte, which plays a characteristic role in a certain tissue type, and may or may not retain the capacity to proliferate further.

### Induced Pluripotent Stem Cells

In some embodiments, genetically engineered human cells described herein can be induced pluripotent stem cells (iPSCs). Without wishing to be bound by any particular theory, the present disclosure contemplated that an advantage of using iPSCs is that the cells can be derived from the same subject to which the progenitor cells are to be administered. That is, for example, a somatic cell can be obtained from a subject, reprogrammed to an induced pluripotent stem cell, and then re-differentiated into a progenitor cell to be administered to the subject (e.g., autologous cells). Because the progenitors are essentially derived from an autologous source, the risk of engraftment rejection or allergic response can be reduced compared to the use of cells from another subject or group of subjects. In addition, the use of iPSCs negates the need for cells obtained from an embryonic or other source. Accordingly, in some embodiments, cells used in accordance with the present disclosure are autologous cells. In some embodiments, cells used in accordance with the present disclosure are stem cells. In some embodiments, cells used in accordance with the present disclosure are not embryonic stem cells.

In some embodiments, differentiation of a cell is generally irreversible under most physiological contexts; however, several methods have been recently developed to reprogram somatic cells to iPSCs. Such methods are known to those of skill in the art and briefly described, in part, herein. For example, in some embodiments, a cell is reprogrammed. In some such embodiments, reprogramming involves a change that alters or reverses a differentiation state of a given differentiated cell (e.g., a somatic cell), for example, into an undifferentiated or more primitive type of cell. In some embodiments, such reprogrammed or dedifferentiated cells may gain potential to differentiate into several different types of cells under appropriate conditions. It will be understood by those of skill in the art that an act of placing many primary cells in culture can lead to some loss of fully differentiated characteristics. Accordingly, such culturing alone does not render these cells non-differentiated cells (e.g., undifferentiated cells) or pluripotent cells. Rather, a transition of a differentiated cell to a more primitive state (e.g., pluripotency) requires certain reprogramming stimulus/stimuli beyond that which can lead to partial loss of differentiated character in culture. In some embodiments, reprogrammed cells also have the characteristic of the capacity of extended passaging without loss of growth potential, relative to primary cell parents, which generally have capacity for only a limited number of divisions in culture.

In some embodiments, a cell to be reprogrammed can be either partially or terminally differentiated prior to reprogramming. In some embodiments, reprogramming can encompass complete reversion of the differentiation state of a differentiated cell (e.g., a somatic cell) to a pluripotent state or a multipotent state. In some embodiments, reprogramming can encompass complete or partial reversion of the differentiation state of a differentiated cell (e.g., a somatic cell) to an undifferentiated cell (e.g., an embryonic-like cell). In some embodiments, reprogramming can result in expression of particular genes by the cells, the expression of which further contributes to reprogramming. In some embodiments, reprogramming of a differentiated cell (e.g., a somatic cell) can cause the differentiated cell to assume an undifferentiated state (e.g., is an undifferentiated cell), which may be referred to as an induced pluripotent stem cell (iPSC).

In some embodiments, reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a myogenic stem cell). Reprogramming is also distinct from promoting the self-renewal or proliferation of cells that are already pluripotent or multipotent, although, in some embodiments, the compositions and methods described herein can also be of use for such purposes.

One of ordinary skill in the art will be aware of many methods known to generate pluripotent stem cells from somatic cells. Any such method that reprograms a somatic cell to the pluripotent phenotype would be appropriate for use in accordance with the present disclosure.

In addition, reprogramming methodologies for generating pluripotent cells using defined combinations of transcription factors have been described and will be known to those of skill in the art. For example, mouse somatic cells can be converted to ES cell-like cells with expanded developmental potential by the direct transduction of Oct4, Sox2, Klf4, and c-Myc; see, e.g., Takahashi and Yamanaka, Cell 126(4): 663-76 (2006). In some embodiments, iPSCs resemble ES cells, as they restore the pluripotency-associated transcriptional circuitry and much of the epigenetic landscape. In some embodiments, mouse iPSCs satisfy all standard assays for pluripotency known to those in the art. For example, mouse iPSCs are capable of in-vitro differentiation into cell types of the three germ layers, teratoma formation, contribution to chimeras, germline transmission (see, e.g., Maherali and Hochedlinger, 2008, Cell Stem Cell. 3(6):595-605), and tetraploid complementation.

In some embodiments, human iPSCs can be obtained using similar transduction methods, and the transcription factor trio, OCT4, SOX2, and NANOG, has been established as the core set of transcription factors that govern pluripotency; see, e.g., Budniatzky and Gepstein, Stem Cells Transl Med. 3(4):448-57 (2014); Barrett et al, Stem Cells Trans Med 3: 1-6 sctm.2014-0121 (2014); Focosi et al., Blood Cancer Journal 4: e211 (2014); and references cited therein. In some embodiments, production of iPSCs can be achieved by the introduction of nucleic acid sequences encoding stem cell-associated genes into an adult, somatic cell, historically using viral vectors.

In some embodiments, iPSCs can be generated or derived from terminally differentiated somatic cells, as well as from adult stem cells, or somatic stem cells. That is, for example, a non-pluripotent progenitor cell can be rendered pluripotent or multipotent by reprogramming. In some such embodiments, it may not be necessary to include as many reprogramming factors as required to reprogram a terminally differentiated cell. Further, in some embodiments, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 7(5):618-30 (2010).

In some embodiments, reprogramming can be achieved by introducing a combination of nucleic acids encoding stem cell-associated genes, including, for example, Oct-4 (also known as Oct-3/4 or Pouf51), Sox1, Sox2, Sox3, Sox 15, Sox 18, NANOG, KM, Klf2, Klf4, Klf5, NR5A2, c-Myc, 1-Myc, n-Myc, Rem2, Tert, and LIN28. Reprogramming in accordance with the present disclosure can further comprise introducing one or more of Oct-3/4, a member of the Sox family, a member of the Klf family, and a member of the Myc family to a somatic cell. In some embodiments, such reprogramming may further comprise introducing one or more of each of Oct-4, Sox2, Nanog, c-MYC and Klf4 for reprogramming. It will be appreciated by one of skill in the art that, the exact method used for reprogramming is not necessarily critical to technologies provided by the present disclosure. However, where cells differentiated from the reprogrammed cells are to be used in, e.g., human therapy, in some embodiments, reprogramming is not effected by a method that alters the genome. Thus, in some such embodiments, reprogramming can be achieved, e.g., without the use of viral or plasmid vectors.

In some embodiments, efficiency of reprogramming (i.e., the number of reprogrammed cells) derived from a population of starting cells can be enhanced by the addition of various agents, e.g., small molecules, as shown by Shi et al, Cell-Stem Cell 2:525-528 (2008); Huangfu et al, Nature Biotechnology 26(7):795-797 (2008) and Marson et al, Cell-Stem Cell 3 132-135 (2008). For example, in some embodiments, an agent or combination of agents that enhance the efficiency or rate of induced pluripotent stem cell production can be used in the production of patient-specific or disease-specific iPSCs. Examples of agents that enhance reprogramming efficiency include, but are not limited to, soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), Suberoylanilide Hydroxamic Acid (SAHA (e.g., MK0683, vorinostat) and other hydroxamic acids), BML-210, Depudecin (e.g., (-)-Depudecin), HC Toxin, Nullscript (4-(1,3-Dioxo-lH, 3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pi valoyloxy methyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido) caproic hydroxamic acid), AOE (2-amino-8-oxo-9, 10-epoxydecanoic acid), CHAP31 and CHAP 50, dominant negative forms of the HDACs (e.g., catalytically inactive forms), siRNA inhibitors of the HDACs, and antibodies that specifically bind to the HDACs. As will be appreciated by one of skill in the art, such agents are generally available, including, but not limited to, commercial sources such as BIOMOL International, Fukasawa, Merck Biosciences, Novartis, Gloucester Pharmaceuticals, Titan Pharmaceuticals, MethylGene, and Sigma Aldrich.

In some embodiments, to confirm induction of pluripotent stem cells for use in accordance with the present disclosure, isolated clones can be tested for the expression of a stem cell marker. In some embodiments, such expression in a cell derived from a somatic cell identifies the cells as induced pluripotent stem cells. In some embodiments, stem cell markers include, but are not limited to, SSEA3, SSEA4, CD9, Nanog, Fbxl5, Ecatl, Esgl, Eras, Gdf3, Fgf4, Cripto, Daxl, Zpf296, Slc2a3, Rexl, Utfl, and Natl. For example, in some embodiments, a cell that expresses Oct4 or Nanog is identified as pluripotent. As will be appreciated by one of skill in the art, methods for detecting the expression of such markers can include, for example, RT-PCR and immunological methods that detect the presence of the encoded polypeptides, such as Western blots or flow cytometric analyses. In some embodiments, intracellular markers may be best identified via RT-PCR, or protein detection methods such as western blot or immunocytochemistry, while cell surface markers may be most readily identified and/or localized, e.g., by immunocytochemistry.

In some embodiments, pluripotent stem cell character of isolated cells can be confirmed by tests evaluating the ability of the iPSCs to differentiate into cells of each of the three germ layers. For example, in some embodiments, teratoma formation in nude mice can be used to evaluate pluripotent character of isolated clones. In some such embodiments, cells can be introduced into nude mice and histology and/or immunohistochemistry can be performed on a tumor arising from the introduced cells; growth of a tumor comprising cells from all three germ layers, for example, further indicates that the cells are pluripotent stem cells.

### Cells of the Peripheral Nervous System

The peripheral nervous system includes neurons, nerves, and other nervous system cells (e.g., glia, e.g., Schwann cells) that are outside of the central nervous system (brain and spinal cord). Neurons, which process information, and glial cells, which provide mechanical and metabolic support to the nervous system, are the two main classes of cells of the peripheral nervous system. In some embodiments, neurons include, but are not limited to, sensory neurons (collect impulses from the sensory receptors in areas such as skin, muscles, and organs and carries those impulses through the nerves to the CNS) and motor neurons (collect outgoing messages from the CNS and delivers them to the appropriate body organs, instructing them what action needs to be taken). In some embodiments, glial cells include, but are not limited to Schwann cells in and/or apposed to or contacting nerves or satellite cells in ganglia. In some embodiments, genetically engineered cells of the present disclosure may be neurons and nerves of the peripheral nervous system. In some such embodiments, neurons and nerves are human neurons and nerves.

Generation and/or Isolation of Cells for Genetic Modification
Creating Patient Specific iPSCs

In some embodiments of the present disclosure, patient specific cells are harvested. In some embodiments, such harvested cells may be used to create or derive one or more types of progenitor or stem cells. For example, in some embodiments, patient specific iPS cell, cells, or cell lines are generated. As is known to one of skill in the art, there are many methods for creating patient specific iPS cells (e.g., for non-limiting examples, see Takahashi and Yamanaka 2006; Takahashi, Tanabe et al. 2007). For example, in some embodiments, a generating or creating step can comprise: i) isolating a somatic cell, such as a skin cell or fibroblast, from the patient; and ii) introducing a set of pluripotency-associated genes into the somatic cell in order to induce the cell to become a pluripotent stem cell. As described herein, in some embodiments, the set of pluripotency-associated genes can be one or more of OCT4, SOX1, SOX2, SOX3, SOX15, SOX18, NANOG, KLF1, KLF2, KLF4, KLF5, c-MYC, n-MYC, REM2, TERT and LIN28.

### Isolating a Mesenchymal Stem Cell

In some embodiments, mesenchymal stem cells can be isolated according to any method known in the art, such as from a patient's bone marrow or peripheral blood. For example, in some embodiments, marrow aspirate can be collected into a syringe with heparin. Cells can be washed and centrifuged on a Percoll. The cells can be cultured in Dulbecco's modified Eagle's medium (DMEM) (low glucose) containing 10% fetal bovine serum (FBS), such as in, e.g., Pittinger M F, Mackay A M, Beck S C et al., Science 1999; 284: 143-147.

Biopsy and Isolation of Tissue and/or Cells from Subjects

As described herein, in some embodiments, one or more cells is harvested from a subject. In some such embodiments, cells may be obtained through a biopsy (sample of tissue) or aspirate (sample of fluid) that is removed from the body of a subject. There are many different kinds of biopsies or aspirates. If the biopsy will be performed with any type of sharp tool and/or will be on the skin or other sensitive area, numbing medicine can be applied first. A biopsy or aspirate may be performed according to any of the known methods in the art. For example, in some embodiments, an aspirate is a bone marrow aspirate and a large needle is used to enter the pelvis bone to collect bone marrow. In some embodiments, a nerve biopsy is performed with tissue taken from skin or leg to isolate neurons of the peripheral nervous system. In some such embodiments, the nerve segment is excised inflicting minimal mechanical injury, for example, squeezing or stretching the nerve is avoided and excessive removal of fat or connective tissue is not attempted.

In some embodiments, biopsy samples are processed to isolate one or more cells. In some such embodiments, an isolated cell is a cell that has been removed from an organism in which it was originally found, or a descendant of such a cell. In some embodiments, the cell can be cultured in-vitro, e.g., under defined conditions or in the presence of other cells. In some embodiments, the cell can be later introduced into a second organism or re-introduced into the organism from which it (or the cell from which it is descended) was isolated. In some embodiments, an isolated population of cells comprises a population of cells that has been removed and separated from a mixed or heterogeneous population of cells. In some embodiments, an isolated population can be a substantially pure population of cells, as compared to a heterogeneous population from which the isolated population of cells was isolated or enriched. In some embodiments, the isolated population can be an isolated population of human progenitor cells, e.g., a substantially pure population of human progenitor cells, as compared to a heterogeneous population of cells comprising human progenitor cells and cells from which the human progenitor cells were derived.

In some embodiments, a population of cells is considered substantially enhanced relative to pre-existing or reference levels. In some embodiments, the increase is at least 2-fold, at least 3-, 4-, 5-, 6-, 7-, 8-, 9, 10-, 20-, 30-, 40-, 50-, 60-, 70-, 80-, 90-100-, 150-, 200-, 250-, 300-, 350-, 400-, 450-, 500-, 600-, 700-, 800-, 900-, 1000-, 5000-, 10000-, 20000-, 50000-, 100000- or more fold depending, e.g., on the desired levels of such cells for ameliorating pain.

In some embodiments, a population or type of cell is considered substantially enriched when it accounts for at least about 10%, 20%, 30%, 40%, 50%, 60%, 70%, 80%, 90% or more with respect to total number of cells making up a given cell population.

In some embodiments, a population of cells is considered substantially pure with respect to a particular cell population, when the population is at least about 75%, 85%, 90%, 95% pure, with respect to the cells making up a total cell population. For example, in some embodiments, the terms “substantially pure” or “essentially purified,” with regard to a population of progenitor cells, describe a population of cells that contain fewer than about 20%, about 15%, about 10%, about 9%, about 8%, about 7%, about 6%, about 5%, about 4%, about 3%, about 2%, about 1%, or less than 1%), of cells that are not progenitor cells as described herein.

### Isolating Cells of the Peripheral Nervous System

In some embodiments, neurons of the peripheral nervous system may be isolated according to any known method in the art. For example, in some embodiments, a nerve segment is excised inflicting minimal mechanical injury under aseptic conditions. Squeezing or stretching the nerve is strictly avoided and excessive removal of fat or connective tissue is not attempted. Since nerve fibers are generally very sensitive to mechanical injury, in some embodiments, a proximal nerve cut is performed first, when cutting a nerve segment for removal. By way of non-limiting example, following harvest, a nerve segment is processed using mechanical and chemical steps. In some embodiments following the isolation, the outermost connective tissue layer, the epineurium, is removed and collected for enzymatic digestion. In some embodiments, nerve fibers are teased apart with the help of fine forceps until all fascicles are separated into individual fibers. In some embodiments, epineurium and teased fibers are then subjected to enzymatic digestion overnight with dispase II and type I collagenase. The digested products are filtered and collected by centrifugation and the resulting cell suspensions are plated onto adhesive substrates (e.g. PLL/laminin). Adherent cells may be cultured for analysis such as in, e.g., Andersen et al., Scientific Reports-Nature, 2016, 6:31781. In some embodiments, one or more neurons or progenitors is isolated. In some embodiments, a population of neurons and/or progenitors is isolated.

### Genetically Modified Cells

In some embodiments, a genetically modified cell comprises at least one genetic modification introduced by gene editing (e.g., using the CRISPR/Cas9 or CRISPR/Cpf1 system). Such gene editing techniques are described herein and are known to those of skill in the art. In some embodiments, a genetically modified cell can be a genetically modified stem cell. In some embodiments, a genetically modified cell can be a genetically modified progenitor cell. In some embodiments, a genetically modified cell can be a genetically modified neuron or neuronal progenitor of the peripheral nervous system. In some embodiments, a genetically modified cell comprising an exogenous genome-targeting nucleic acid and/or an exogenous nucleic acid encoding a genome-targeting nucleic acid is contemplated herein.

In some embodiments, such as, for example, methods comprising ex-vivo modification of cells, a control treated population of cells is used. In some such embodiments, control treated cells are treated with identical media, viral induction, nucleic acid sequences, temperature, confluency, flask size, pH, etc., with the exception of the addition of one or more gene editing components. Any method known in the art can be used to measure transcription of SCN9A and/or SCN10A gene or NaV1.7 and/or NavV1.8 protein expression or activity, for example Western Blot analysis of the SCN9A and/or SCN10A protein or real time PCR for quantifying SCN9A or SCN10A mRNA.

In some embodiments, technologies of the present disclosure may use one or more design elements to achieve precise and selective targeting of pain-causative and/or pain propagating neurons. For example, in some embodiments, the present disclosure provides design elements comprising, e.g., (i) localized delivery of a non-replicative viral vector that requires synaptic terminals, sparing the bulk of somatic tissues near the pain site; (ii) neuron-specific promoters that drive expression of the gene editing construct; and/or (iii) gRNA programmed targeting of non-essential ion channel genes exclusively expressed by DRG neurons to spare other types of neurons (e.g., efferent neurons, interneurons, etc.).

In some embodiments, the present disclosure provides technologies for editing a polynucleotide encoding an ion channel or subunit thereof in a cell. In some embodiments, the cell is a stem cell or progenitor cell. In some embodiments, the stem cell is an iPSC, mesenchymal stem cell, or neural stem cell. In some embodiments, the progenitor cell is a neuronal progenitor cell. In some embodiments, the progenitor cell is a glial progenitor cell. In some embodiments, the progenitor cell is a glial and/or neuronal progenitor cell. In some embodiments, the cell is a glial cell. In some embodiments, the glial cell is a Schwann cell or satellite glial cell. In some embodiments, the cell is a neuron. In some embodiments, the neuron is a DRG neuron.

Differentiation of Genome-Edited iPSCs into Cells of the Peripheral Nervous System

In some embodiments, the present disclosure provides, among other things, a method of differentiating cells using iPSCs. For example, in some embodiments, the present disclosure provides an ex-vivo approach for differentiating genome-edited iPSCs into cells of peripheral nervous system lineage and/or identity (e.g., a neuron or neuronal progenitor such as a DRG or progenitor thereof, or a glial cell such as Schwann cell in nerves or satellite glial cell in ganglia). A differentiating step may be performed according to any method known in the art.

For example, in some embodiments, neuronal differentiation of iPSCs is induced using a combination of factors (e.g., brain-derived neurotrophic factor (BDNF), glial cell-derived neurotrophic factor (GDNF), nerve growth factor (NGF) and dibutyryl cyclic AMP (dbcAMP)). In some such embodiments, the iPSCs-derived neuronal cells may be further differentiated into Schwann cells using ciliary neurotrophic factor (CNTF), neuregulin 10 and dbcAMP (see, e.g., Wang et al., Biomaterials. 2011; 32(22): 5023-5032).

In some embodiments, differentiation does not result in a post-mitotic neuronal cell. For example, in some such embodiments, differentiation may result in a neuronally-committed progenitor cell.

Differentiation of Genome-Edited Mesenchymal Stem Cells into Cells of the Peripheral Nervous System

In some embodiments, the present disclosure provides, among other things, a method of differentiating cells using mesenchymal stem cells (MSCs). For example, in some embodiments, the present disclosure provides an ex-vivo approach for differentiating genome-edited mesenchymal stem cells into cells of peripheral nervous system lineage and/or identity (e.g., a neuron or neuronal progenitor, such as a DRG cell or progenitor thereof, or a glial cell such as Schwann cell in nerves or satellite glial cell in ganglia). A differentiating step may be performed according to any method known in the art. For example, in some embodiments, MSCs are treated with various factors and/or hormones, including, e.g., basic fibroblast growth factor, human recombinant platelet derived growth factor, forskolin, and glial growth factor-2 (see, e.g., Ladak et al., Experimental Neurology 228 (2011) 242-252).

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Table 1 sets forth several, non-limiting examples of genome editing system implementations. Those of skill in the art will appreciate, however, that these listings are not comprehensive, and that other implementations are possible. With reference to Table 1 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 1
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described |
| | | | above plus a single-stranded or |
| | | | double stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both a |
| | | | gRNA and a donor template. |
| DNA | | | A DNA or DNA vector encoding |
| | | | an RNA-guided nuclease, a gRNA |
| | | | and a donor template. |
| DNA | DNA | [N/A] | Two separate DNAs, or two |
| | | | separate DNA vectors, encoding |
| | | | the RNA-guided nuclease and the |
| | | | gRNA, respectively. |
| DNA | DNA | DNA | Three separate DNAs, or three |
| | | | separate DNA vectors, encoding |
| | | | the RNA-guided nuclease, the |
| | | | gRNA and the donor template, |
| | | | respectively. |
| DNA | | [N/A] | A DNA or DNA vector encoding |
| | | | an RNA-guided nuclease and a |
| | | | gRNA |
| DNA | | DNA | A first DNA or DNA vector |
| | | | encoding an RNA-guided nuclease |
| | | | and a gRNA, and a second DNA or |
| | | | DNA vector encoding a donor |
| | | | template. |
| DNA | DNA | | A first DNA or DNA vector |
| | | | encoding an RNA-guided nuclease |
| | | | and second DNA or DNA vector |
| | | | encoding a gRNA and a donor |
| | | | template. |
| DNA | | | A first DNA or DNA vector |
| | DNA | | encoding an RNA-guided nuclease |
| | | | and a donor template, and a second |
| | | | DNA or DNA vector encoding a |
| | | | gRNA |
| DNA | | | A DNA or DNA vector encoding |
| | RNA | | an RNA-guided nuclease and a |
| | | | donor template, and a gRNA |
| RNA | | [N/A] | An RNA or RNA vector encoding |
| | | | an RNA-guided nuclease and |
| | | | comprising a gRNA |
| RNA | | DNA | An RNA or RNA vector encoding |
| | | | an RNA-guided nuclease and |
| | | | comprising a gRNA, and a DNA or |
| | | | DNA vector encoding a donor |
| | | | template. |

### Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors described herein), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs).

Viral vectors can comprise one or more sequences encoding genome editing system components, such as an RNA-guided nuclease, a gRNA and/or a donor template. A viral vector can also comprise a sequence encoding a signal peptide (e.g., for nuclear localization, nucleolar localization, or mitochondrial localization), associated with (e.g., inserted into or fused to) a sequence coding for a protein. As one example, a viral vectors can include a Cas9 coding sequence that includes one or more nuclear localization sequences (e.g., a nuclear localization sequence from SV40).

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in WO 2016/073990. Any suitable nanoparticle design can be used to deliver genome editing system components or nucleic acids encoding such components. For instance, organic (e.g. lipid and/or polymer) nonparticles can be suitable for use as delivery vehicles in certain embodiments of this disclosure. Exemplary lipids for use in nanoparticle formulations, and/or gene transfer are shown in Table 2, and Table 3 lists exemplary polymers for use in gene transfer and/or nanoparticle formulations.

TABLE 2
Lipids Used for Gene Transfer

| Lipid | Abbreviation | Feature |
|---|---|---|
| 1,2-Dioleoyl-sn-glycero-3- | DOPC | Helper |
| phosphatidylcholine | | |
| 1,2-Dioleoyl-sn-glycero-3- | DOPE | Helper |
| phosphatidylethanolamine | | |
| Cholesterol | | Helper |
| N-[1-(2,3-Dioleyloxy)propyl]N,N,N- | DOTMA | Cationic |
| trimethylammonium chloride | | |
| 1,2-Dioleoyloxy-3- | DOTAP | Cationic |
| trimethylammonium-propane | | |
| Dioctadecylamidoglycylspermine | DOGS | Cationic |
| N-(3-Aminopropyl)-N,N-dimethyl- | GAP-DLRIE | Cationic |
| 2,3-bis(dodecyloxy)-1- | | |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido- | DOSPA | Cationic |
| ethyl]-N,N-dimethyl- | | |
| 1-propanaminium trifluoroacetate | | |
| 1,2-Dioleyl-3-trimethylammonium-propane | DOPA | Cationic |
| N-(2-Hydroxyethyl)-N,N-dimethyl- | MDRIE | Cationic |
| 2,3-bis(tetradecyloxy)-1- | | |
| propanaminium bromide | | |
| Dimyristooxypropyl dimethyl | DMRI | Cationic |
| hydroxyethyl ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium | | |
| chloride | | |
| rac-[2(2,3-Dihexadecyloxypropyl- | CLIP-6 | Cationic |
| oxymethyloxy)ethyl]trimethylammonium | | |
| bromide | | |
| Ethyldimyristoylphosphatidylcholine | EDMPC | Cationic |
| 1,2-Distearyloxy-N,N-dimethyl- | DSDMA | Cationic |
| 3-aminopropane | | |
| 1,2-Dimyristoyl-trimethylammonium | DMTAP | Cationic |
| propane | | |
| O,O′-Dimyristyl-N-lysyl aspartate | DMKE | Cationic |
| 1,2-Distearoyl-sn-glycero-3- | DSEPC | Cationic |
| ethylphosphocholine | | |
| N-Palmitoyl D-erythro-sphingosyl | CCS | Cationic |
| carbamoyl-spermine | | |
| N-t-Butyl-N0-tetradecyl-3- | diC14-amidine | Cationic |
| tetradecylaminopropionamidine | | |
| Octadecenolyoxy[ethyl-2- | DOTIM | Cationic |
| heptadecenyl-3 hydroxyethyl] | | |
| imidazolinium chloride | | |
| N1-Cholesteryloxycarbonyl-3,7- | CDAN | Cationic |
| diazanonane-1,9-diamine | | |
| 2-(3-[Bis(3-amino-propyl)- | RPR209120 | Cationic |
| amino]propylamino)-N- | | |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl- | DLin-KC2- | Cationic |
| [1,3]-dioxolane | DMA | |
| dilinoleyl-methyl-4-dimethylaminobutyrate | DLin-MC3- | Cationic |
| | DMA | |

TABLE 3
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in WO 2016/073990. In-vitro, RNA-guided nuclease-encoding and/or gRNA-encoding RNA can be delivered, e.g., by microinjection, electroporation, transient cell compression or squeezing. Lipid-mediated transfection, peptide-mediated delivery, GalNAc- or other conjugate-mediated delivery, and combinations thereof, can also be used for delivery in-vitro and in-vivo.

In-vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

### Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In some embodiments, compositions described herein comprise AAV vectors at known vector genome concentrations and/or for a final vector genome delivery concentration. In some embodiments, the total vector genome titer administered is between about 1×10⁹ and about 1×10¹⁴ VG, e.g., 1×10⁹ VG, about 1×10¹⁰ VG, about 1×10¹¹VG, about 1×10¹²VG, about 1×10¹³ VG, or about 1×10¹⁴ VG.

In some embodiments, compositions described herein are delivered as a liquid medium and/or solution. In some embodiments, delivery of specific volumes are known to be within safe and/or efficacious ranges. In some embodiments, a volume for delivery is between about 1 μl and about 100 μl. In some embodiments, a volume for delivery is about 1 μl, 2 μl, 3 μl, 4 μl, 5 μl, 6 μl, 7 μl, 8 μl, 9 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 31 μl, 32 μl, 33 μl, 34 μl, 35 μl, 36 μl, 37 μl, 38 μl, 39 μl, 40 μl, 41 μl, 42 μl, 43 μl, 44 μl, 45 μl, 46 μl, 47 μl, 48 μl, 49 μl, 50 μl, 51 μl, 52 μl, 53 μl, 54 μl, 55 μl, 56 μl, 57 μl, 58 μl, 59 μl, 60 μl, 61 μl, 62 μl, 63 μl, 64 μl, 65 μl, 66 μl, 67 μl, 68 μl, 69 μl, 70 μl, 71 μl, 72 μl, 73 μl, 74 μl, 75 μl, 76 μl, 77 μl, 78 μl, 79 μl, 80 μl, 81 μl, 82 μl, 83 μl, 84 μl, 85 μl, 86 μl, 87 μl, 88 μl, 89 μl, 90 μl, 91 μl, 92 μl, 93 μl, 94 μl, 95 μl, 96 μl, 97 μl, 98 μl, 99p1, or 100 μl.

In some embodiments, compositions as described herein are administered locally directly to Dorsal root ganglion (DRG) neurons. In some embodiments, delivery is between the L1 and S3 segments of the spinal cord. In some embodiments, delivery is at the L1 segment. In some embodiments, delivery is at the L2 segment. In some embodiments, delivery is at the L3 segment. In some embodiments, delivery is at the L4 segment. In some embodiments, delivery is at the L5 segment. In some embodiments, delivery is at the S1 segment. In some embodiments, delivery is at the S2 segment. In some embodiments, delivery is at the S3 segment. In some embodiments, delivery is at the transforaminal space surrounding the DRG (L1-S3).

In some embodiments, local administration is achieved through precision injection. In some embodiments, precision injection may involve only one needle, wherein the needle has a gauge of between about 18G and about 35G, e.g., about 16G, 17G, 18G, 19G, 20G, 21G, 22G, 23G, 24G, 25G, 26G, 27G, 28G, 29G, 30G, 31G, 32G, 33G, 34G, or 35G. In some embodiments, precision injection may involve the use of two needles. In some embodiments, the gauge of these needles can range from 18-35G. In some embodiments, a first needle is utilized as a guide needle and is used to approach the site for local administration. In some embodiments, a guide needle has a gauge in the range of 16-26G, e.g., about 16G, 17G, 18G, 19G, 20G, 21G, 22G, 23G, 24G, 25G, or 26G. In some embodiments, a second needle is utilized as a penetrating needle, where such penetrating needle is of a finer gauge than the first needle and is will be threaded through the guide needle to penetrate the tissue of interest. In some embodiments, a penetrating needle has a gauge in the range of 26-35G, e.g., about 26G, 27G, 28G, 29G, 30G, 31G, 32G, 33G, 34G, or 35G. Needle tips for use in precision injection are known in the art, any of which can be used in methods of the disclosure. Nonlimiting needle tips include, e.g., a Quincke, Whitacre, Pitkin, Greene, Tuohy, Pencil-point, Sprotte needle tip and/or a blunt-ended needled tip. In some embodiments, wherein a guide needle is utilized for precision injection, the guide needle tip may be but is not limited to a Quincke, Pitkin, and/or Tuohy needle tip. In some embodiments, wherein a penetrating needle is utilized for precision injection, the penetrating needle tip may be a pencil-point and/or Whitacre needle tip.

In some embodiments, compositions comprising AAV particles and constructs as described herein are locally administered (e.g., directly to DRG neurons) utilizing a controlled flow rate. In some embodiments, a controlled flow rate may be between 0.2 μl/min to 10 μl/min, e.g., a controlled flow rate may be 0.2 μl/min, 0.3 μl/min, 0.4 μl/min, 0.5 μl/min, 0.6 μl/min, 0.7 μl/min, 0.8 μl/min, 0.9p1/min, 1.0 μl/min, 1.25 μl/min, 1.5 μl/min, 1.75 μl/min, 2 μl/min, 2.25 μl/min, 2.5 μl/min, 2.75 μl/min, 3 μl/min, 3.5 μl/min, 4 μl/min, 4.5 μl/min, 5 μl/min, 5.5 μl/min, 6 μl/min, 6.5 μl/min, 7 μl/min, 7.5 μl/min, 8 μl/min, 8.5 μl/min, 9 μl/min, 9.5 μl/min and/or 10 μl/min. In some embodiments, a controlled flow rate may be controlled by an electronic and/or mechanical pump.

In some embodiments, compositions comprising AAV particles and constructs as described herein are administered via injection. In some embodiments, injection occurs utilizing one continuous flow rate over a period of time. In some embodiments, injection occurs in a staggered and/or stepped delivery pattern (e.g., at a relative high-flowrate, at a relative low flowrate (e.g., zero), and again at a relative high-flowrate, this cycle may be repeated any number of times as appropriate).

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein.

### Multi-Modal or Differential Delivery of Components

Skilled artisans will appreciate, in view of the instant disclosure, that different components of genome editing systems disclosed herein can be delivered together or separately and simultaneously or nonsimultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule or gRNA. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### Administration of Modified Cells

In some embodiments, modified cells, e.g., progenitor cells (e.g., genetically modified progenitor cells) are administered to a subject. In some embodiments, a composition of modified cells can contain a physiologically tolerable carrier together with a cell described herein. In some embodiments, such compositions may further comprise at least one additional bioactive agent as described herein. In some embodiments, the at least one additional bioactive agent may be dissolved or dispersed therein as an active ingredient. In some embodiments, a composition is not substantially immunogenic when administered to a mammal or human patient for therapeutic purposes, unless so desired.

In general, compositions (e.g., comprising modified cells (e.g., progenitor cells)) may be administered as a suspension with a pharmaceutically acceptable carrier. One of skill in the art will recognize that a pharmaceutically acceptable carrier to be used in a cell composition will not any agents (e.g., certain buffers, compounds, cryopreservation agents, preservatives, etc.) that substantially interfere with the viability of the cells to be delivered to the subject.

In some embodiments, a formulation comprising cells can include e.g., osmotic buffers that permit cell membrane integrity to be maintained. In some such embodiments, nutrients to maintain cell viability or enhance engraftment upon administration. In some such embodiments, such formulations and suspensions are known to those of skill in the art and/or can be adapted for use with compositions comprising cells (e.g., progenitor cells), as described herein. In some embodiments, a composition comprising cells may also be emulsified or presented as a liposome composition, provided that emulsification procedure does not adversely affect cell viability. In some such embodiments, the composition comprising cells and any other active ingredient may be mixed with excipients that are pharmaceutically acceptable and compatible with the active ingredient, and in amounts suitable for use in the therapeutic methods described herein.

In some embodiments, additional agents included in a composition comprising cells as describe herein may include pharmaceutically acceptable salts of the components therein. Pharmaceutically acceptable salts may include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. In some embodiments, exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. In some embodiments, liquid compositions can also contain liquid phases in addition to and to the exclusion of water. By way of non-limiting example, in some such embodiments, additional liquid phases are glycerin, vegetable oils such as cottonseed oil, and water-oil emulsions.

In some embodiments, a composition comprises a least 10² progenitor cells, at least 5×10²progenitor cells, at least 10³progenitor cells, at least 5×10³progenitor cells, at least 10⁴progenitor cells, at least 5×10⁴ progenitor cells, at least 10⁵ progenitor cells, at least 2×10⁵ progenitor cells, at least 3×10⁵ progenitor cells, at least 4×10⁵ progenitor cells, at least 5×10⁵ progenitor cells, at least 6×10⁵ progenitor cells, at least 7×10⁵ progenitor cells, at least 8×10⁵ progenitor cells, at least 9×10⁵ progenitor cells, at least 1×10⁶ progenitor cells, at least 2×10⁶ progenitor cells, at least 3×10⁶ progenitor cells, at least 4×10⁶ progenitor cells, at least 5×10⁶ progenitor cells, at least 6×10⁶ progenitor cells, at least 7×10⁶ progenitor cells, at least 8×10⁶ progenitor cells, at least 9×10⁶ progenitor cells, or multiples thereof. In some embodiments, progenitor cells can be derived from one or more donors. In some embodiments, progenitor cells are obtained from an autologous source. In some embodiments, progenitor cells are obtained from stem cells (e.g., iPSC cells that are differentiated into progenitor cells). In some embodiments, progenitor cells can be expanded in culture prior to administration to a subject.

### Methods of Treatment

In some embodiments, treatment according to the present disclosure can ameliorate or prevent one or more symptoms associated with a pain disorder and/or disorder associated with pain by decreasing or altering the amount of an SCN9A and/or SCN10A gene or gene product (e.g., protein) in a subject.

The present disclosure provides, in some embodiments, methods for treating a subject with one or more symptoms of a pain disorder and/or disorder associated with pain, or a patient at risk of a pain disorder and/or disorder associated with pain.

In some embodiments, a method is or comprises ex-vivo cell-based therapy. For example, in some embodiments, a biopsy is performed on a patient. In some embodiments, the biopsy is of a patient's peripheral nerves is performed. In some embodiments, nerve tissue can be isolated from the patient's skin or leg. Then, at least one cell of the peripheral nervous system (e.g., a neuron or a glial cell such as Schwann cell in nerves or satellite glial cell in ganglia, or a progenitor thereof) is isolated from the biopsied material. Concomitant with or following isolation, the chromosomal DNA of the cell of the peripheral nervous system (e.g., a neuron and/or a glial cell such as Schwann cell in nerves or satellite glial cell in ganglia) can be edited using approaches and technologies as provided and described herein. Finally, the edited cell of the peripheral nervous system (e.g., a neuron or a glial cell such as Schwann cell in nerves or satellite glial cell in ganglia) is implanted into the patient.

In some embodiments, a patient specific stem cell may be harvested or generated. For example, in some embodiments, a stem cell may be an induced pluripotent stem cell (iPSC) is generated (e.g., from cells harvested from peripheral nerves, patient skin, etc.). In some embodiments, a stem cell may be a mesenchymal stem cell isolated from a patient's bone marrow or peripheral blood. In some embodiments, stem cells are autologous stem cells. In some such embodiments, chromosomal DNA of these iPSC cells can be edited using approaches and technologies provided and described herein. Concomitant with or following isolated, genome-edited iPSCs can be differentiated into cells of the nervous system (e.g., a neural stem cell, a neuronal progenitor cell, a peripheral neuron or a glial cell such as Schwann cell in nerves or satellite glial cell in ganglia). Finally, the neural stem cell, neuronal progenitor cell, or neuronal/glial cells of the peripheral nervous system can be administered (e.g., via infusion or implantation) to a patient (e.g., a subject in need thereof, a control subject, etc.).

In some embodiments, cells produced for ex-vivo therapy are comprehensively analyzed prior to administration to a subject. For example, in some embodiments, nuclease-based therapeutics may have some level of off-target effects. By performing gene editing ex-vivo, cells are able to be analyzed after modification (e.g., editing), but prior to administration (e.g., implantation) to a subject. In some embodiments, the entire genome of one or more edited cells can be sequenced. In some embodiments, such sequencing provides information regarding off-target effects, if any. In some such embodiments, if any off-target effects are detected, cells with effects in genomic locations associated with minimal risk to the patient may be selected for administration to a subject. In some embodiments, populations of specific cells, including clonal populations, can be isolated prior to implantation.

In some embodiments, an advantage of ex-vivo cell therapy relates to genetic modification in iPSCs compared to other primary cell sources. Generally, iPSCs are prolific, such that obtaining the large number of cells that will be required for a cell-based therapy is not difficult. Furthermore, in some embodiments, iPSCs are an ideal cell type for performing clonal isolations. This allows screening for the correct genomic modification, without risking a decrease in viability.

In some embodiments, non-iPSCs such as differentiated cells (e.g., glial cells), are viable for only a few passages and difficult to clonally expand. Thus, manipulation of iPSCs for the treatment of pain can be much easier, and can shorten the amount of time needed to make the desired genetic modification.

In some embodiments, the present disclosure provides, among other things, methods comprising in-vivo therapy. In some such embodiments, chromosomal DNA of the cells in the patient is edited using the technologies provided and described in accordance with the present disclosure. In some embodiments, the target cell in an in-vivo based therapy is a neuron of the peripheral nervous system. In some embodiments, the target cell in an in-vivo based therapy is a neuronal progenitor cell.

In some embodiments comprising in-vivo therapy, impact in non-target cells can also be prevented by the use of promoters only active in certain cells and or developmental stages. In some embodiments, promoters are inducible, and therefore can be temporally controlled if the nuclease is delivered as a plasmid.

In some embodiments, an amount of time that delivered RNA and protein remain in the cell can also be adjusted using treatments or domains added to change the half-life. In some embodiments, use of in-vivo approaches would eliminate a number of treatment steps, but a lower rate of delivery can require higher rates of editing. In some such embodiments, use of In-vivo approaches can eliminate problems and losses from ex-vivo approaches such as e.g. engraftment and post-engraftment integration of neurons and glial cells appropriately into existing neural circuits.

In some embodiments, an advantage of an in-vivo approach can be the ease of therapeutic production and administration. In some embodiments, the same therapeutic approach and therapy will have the potential to be used to treat more than one patient, for example a number of patients who share the same or similar genotype or allele.

In some embodiments, ex-vivo cell therapy typically requires using a patient's own cells, which are isolated, manipulated and returned to the same patient. Accordingly, in some such embodiments, ex-vivo approaches may be more individualized, but less translatable on a large scale than in-vivo approaches.

In some embodiments, the present disclosure provides technologies comprising a cellular method for editing SCN9A and/or SCN10A gene(s) in a cell by gene editing. For example, in some embodiments, a cell can be isolated from subject (e.g., a patient or animal). Then, the chromosomal DNA of the cell can be edited using, among other things, materials and methods described herein.

The present disclosure also provides, among other things, approaches which may, regardless of whether a cellular or ex-vivo or in-vivo method, involve reducing (knock-down) or eliminating (knock-out) the expression of the SCN9A and/or SCN10A gene by introducing one or more modifications, e.g. insertions, deletions, substitutions, mutations, etc. within or near the SCN9A and/or SCN10A gene(s) or other DNA sequences that encode regulatory elements of the SCN9A and/or SCN10A gene(s).

For example, in some embodiments, the knock-down or knock-out strategy can involve disrupting the reading frame in the SCN9A and/or SCN10A gene(s) by introducing random insertions or deletions (indels) that arise due to the imprecise NHEJ repair pathway. This can be achieved by inducing one single stranded break or double stranded break in the SCN9A gene with one or more CRISPR endonucleases and a gRNA (e.g., crRNA+tracrRNA, or sgRNA), or two or more single stranded breaks or double stranded breaks in the SCN9A and/or SCN10A gene(s) with two or more CRISPR endonucleases and two or more sgRNAs. This approach can require development and optimization of sgRNAs for the SCN9A and/or SCN10A gene(s).

In some embodiments, the knock-down or knock-out strategy can also involve deletion of one or more segments within or near the SCN9A and/or SCN10A gene(s) or other DNA sequences that encode regulatory elements of the SCN9A and/or SCN10A gene(s). This deletion strategy requires at least a pair of gRNAs (e.g., crRNA+tracrRNA, or sgRNA) capable of binding to two different sites within or near the SCN9A and/or SCN10A gene(s) and one or more CRISPR endonucleases. The CRISPR endonucleases, configured with the two gRNAs, induce two double stranded breaks at the desired locations. After cleavage, the two ends, regardless of whether blunt or with overhangs, can be joined by NHEJ, leading to the deletion of the intervening segment. In certain aspects, NHEJ repair pathways can lead to insertions, deletions or mutations at the joints.

In addition to the above gene editing strategies, another strategy involves modulating expression, function, or activity of SCN9A and/or SCN10A by editing in the regulatory sequence.

In addition to the editing options listed above, site-specific nucleases, e.g., Cas9 or similar proteins, can be used to target effector domains to the same target sites that can be identified for editing, or additional target sites within range of the effector domain. A range of chromatin modifying enzymes, methylases or demethylases can be used to alter expression of the target gene. One possibility is decreasing the expression of the SCN9A and/or SCN10A protein(s) if a mutation leads to undesirable activity. In some such embodiments, such epigenetic regulation may have some advantages, particularly as they are limited in possible off-target effects.

In some embodiments, a number of types of genomic target sites can be present in addition to the coding and splicing sequences. The regulation of transcription and translation implicates a number of different classes of sites that interact with cellular proteins or nucleotides. Often DNA binding sites of transcription factors or other proteins can be targeted for mutation or deletion to study the role of the site, though they can also be targeted to change gene expression. Sites can be added through non-homologous end joining NHEJ or direct gene editing by homology directed repair (HDR). Increased use of genome sequencing, RNA expression and genome-wide studies of transcription factor binding have increased our ability to identify how the sites lead to developmental or temporal gene regulation. These control systems can be direct or can involve extensive cooperative regulation that can require the integration of activities from multiple enhancers. Transcription factors typically bind 6-12 bp-long degenerate DNA sequences. The low level of specificity provided by individual sites suggests that complex interactions and rules are involved in binding and the functional outcome. Binding sites with less degeneracy can provide simpler means of regulation. Artificial transcription factors can be designed to specify longer sequences that have less similar sequences in the genome and have lower potential for off-target cleavage. In some embodiments, any of these types of binding sites can be mutated, deleted or even created to enable changes in gene regulation or expression (see, e.g., Canver, M. C. et al., Nature (2015)).

Efficacy of a given treatment in accordance with the present disclosure can be determined by the skilled clinician. In some embodiments, a treatment is considered effective if any one or all of the signs or symptoms of or other clinically accepted symptoms or markers of disease are improved or ameliorated. For example, in some embodiments, if a level of SCN9A and/or SCN10A is/are altered in a beneficial manner (e.g., decreased by at least 10%), a treatment may be effective. In some embodiments, efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (i) inhibiting the disease, e.g., arresting, or slowing the progression of symptoms; or (ii) relieving the disease, e.g., causing regression of symptoms; and (ii) preventing or reducing the likelihood of the development of symptoms.

### SCN9A

SCN9A has been associated with one or more symptoms of one or more diseases or disorders having or associated with pain such as, but not limited to, Congenital Pain Insensitivity, Anosmia, As If Personality, Borderline Personality Disorder, Malignant neoplasm of breast, Non-Small Cell Lung Carcinoma, Cold intolerance, Febrile Convulsions, Diabetes, Diabetes Mellitus, Dissociative disorder, Epilepsy, Erythromelalgia, Primary Erythermalgia, Facial Pain, Herpesviridae Infections, Hereditary Sensory Autonomic Neuropathy Type 5, Hyperplasia, Neuralgia, Hereditary Sensory and Autonomic Neuropathies, Degenerative polyarthritis, Pain, Pain in limb, Postoperative Pain, Parkinson Disease, Postherpetic neuralgia, Prostatic Neoplasms, Pruritus, Seizures, Somatoform Disorder, Tobacco Use Disorder, Trigeminal Neuralgia, Synovial Cyst, Chronic pain, Acute onset pain, Paramyotonia Congenita (disorder), Malaise, Sensory Discomfort, Burning Pain, Indifference to pain, Inflammatory pain, Mechanical pain, Scalp pain, Hereditary Motor and Sensory-Neuropathy Type II, Common Migraine, Absence of pain sensation, Malignant neoplasm of prostate, Pain Disorder, Knee Osteoarthritis, Neuropathy, Complex Regional Pain Syndromes, Tonic-clonic seizures, Inherited neuropathies, Prostate carcinoma, Breast Carcinoma, Infantile Severe Myoclonic Epilepsy, Myxoid cyst, Channelopathies, Paroxysmal Extreme Pain Disorder, Painful Neuropathy, Compressive Neuropathies, Congenital Indifference to Pain Autosomal Recessive, Generalized Epilepsy With Febrile Seizures Plus Type 2, Generalized Epilepsy With Febrile Seizures Plus 7, Febrile Seizures Familial 3B, and Small Fiber Neuropathy (Adult-onset is referred to as small fiber neuropathy). (See, e.g., Yang et al., Trends Pharmacol. Sci. 39:258-275 (2018).)

In some embodiments, editing the SCN9A gene using any of the methods described herein may be used to treat, prevent and/or mitigate one or more symptoms of one or more disorders described herein.

The SCN9A gene encodes the alpha subunit of a sodium channel, NaV1.7. NaV1.7 is primarily expressed in sensory neurons and plays a significant role in nociception signaling.

In some embodiments, mutations in the SCN9A gene are known to cause pain perception disorders, including primary erythermalgia, paroxysmal extreme pain disorder, congenital insensitivity to pain, and small fiber neuropathy. For example, humans presenting with homozygous SCN9A loss-of-function mutations may suffer from congenital insensitivity to pain (CIP).

In some embodiments, the SCN9A gene is involved in itching.

In some embodiments, gain-of-function mutations in SCN9A cause congenital pain syndromes, such as primary erythermalgia. In some embodiments, gain-of-function mutations in the SCN9A gene result in spontaneous pain as observed in primary erythermalgia and paroxysmal extreme pain disorder. Thus, in some embodiments, knock-out or knock-down of the SCN9A gene in patients having primary erythromelalgia or paroxysmal extreme pain disorder can be used to treat, prevent and/or mitigate the associated symptoms. In some embodiments, primary erythromelalgia is a rare autosomal dominant disorder characterized by episodes of burning pain in the feet and hands in response to heat and movement. Affected individuals typically develop signs and symptoms in early childhood, although in milder cases symptoms can appear later in life. Management of this condition is mainly symptomatic. Besides avoidance of pain triggers (such as heat, exercise, and alcohol), treatment options include cooling and elevating the extremity, use of anesthetics such as lidocaine and mexilitine, and use of opioid drugs in extreme cases.

In some embodiments, paroxysmal extreme pain disorder is another rare disorder characterized by severe episodic pain in rectal, ocular, and mandibular regions as well as skin redness. Symptoms of this condition often begin in the neonatal period or in the early childhood, and can retain throughout life. Agents for treating chronic neuropathic pain disorders are often used to alleviate the pain episodes caused by the disease. Carbamazepine, a sodium channel blocker, has proven most effective of these treatments.

In some embodiments, technologies of the present disclosure may be combined with one or more other treatments, e.g., gene-editing combined with a pharmacological agent or intervention (e.g., lidocaine, mexilitine, carbamazepine).

### SCN10A

SCN10A has been associated with one or more symptoms of one or more diseases or disorders having or associated with pain such as, but not limited to, Congenital Pain Insensitivity, Asthma, Ataxia, Atrial Fibrillation, Malignant neoplasm of breast, Hypertrophic Cardiomyopathy, Cardiovascular Diseases, Cerebellar Diseases, Dyspepsia, Experimental Autoimmune Encephalomyelitis, Primary Erythermalgia, Heart Block, Hereditary Sensory Autonomic Neuropathy Type 5, Hyperalgesia, Acute Myelocytic Leukemia, Multiple Sclerosis, Neuralgia, Neuroblastoma, Pain, Postherpetic neuralgia, Tobacco Use Disorder, Trigeminal Neuralgia, Ventricular Fibrillation, Synovial Cyst, Sciatic Neuropathy, Chronic pain, Fibrillation, Hematologic Neoplasms, Electrocardiogram: P-R interval, Neuropathy, Breast Carcinoma, Central neuroblastoma, Varicella zoster, Brugada Syndrome (disorder), Myxoid cyst, Channelopathies, Compressive Neuropathies, Paroxysmal Extreme Pain Disorder, High grade atrioventricular block, Small Fiber Neuropathy, and Familial Episodic Pain Syndrome 2. (See, e.g., Dib-Hajj et al., Ann. Rev. Neurosci. 42:87-106 (2019).)

In some embodiments, editing the SCN10A gene using any of the methods described herein may be used to treat, prevent and/or mitigate one or more symptoms of one or more disorders described herein.

The gene SCN10A encodes the alpha subunit of a voltage-gated sodium channel, NaV1.8. NaV1.8 is primarily expressed in the sensory neurons of the dorsal root ganglion and plays an important role in neuropathic pain mechanisms.

In some embodiments, mutations in the SCN10A gene have been found in patients with familial episodic pain syndrome type 2. Familial episodic pain syndrome type 2 is a rare autosomal dominant neurologic disorder characterized by adult-onset of paroxysmal pain in the feet region. The episodes are generally triggered by heat, cold, chemicals and certain surfaces. Patients may also develop hypersensitivity to touch and elevated response to pain stimulus. Currently no treatment is available for this disease. Warmth has been shown to relieve the pain episodes.

In some embodiments, SCN10A gene mutations are also responsible for about five percent of the cases of small fiber neuropathy. Small fiber neuropathy is a condition characterized by severe pain attacks and insensitivity to pain. The pain attacks are usually described as numbness, stabbing or burning, or abnormal skin sensations such as tingling or itchiness. Currently, there is no cure for small fiber peripheral neuropathy. Treatment options include intravenous immunoglobulin (IVIG) and plasmapheresis.

In some embodiments, technologies of the present disclosure may be combined with one or more other treatments, e.g., gene-editing combined with a pharmacological agent or intervention (e.g., IVIG plasmapheresis)

### Pain

Among other things, the present disclosure provides technologies for treatment (e.g., amelioration, prevention) of one or more symptoms of a pain disorder and/or disorder associated with pain. In some embodiments, pain is a symptom of one or more pain disorders or of one or more other diseases with which pain is associated. In some embodiments, pain is the only symptom experienced by a subject. In some embodiments, a subject is at risk of having pain, but has not yet experienced pain. For example, in some embodiments, a subject has one or more mutations known to result in a pain disorder and/or disorder associated with pain as described herein. In some such embodiments, the present disclosure provides technologies that may prevent the subject from experiencing pain as related to manifestation of the disorder which they are at risk of developing.

In some embodiments, one or more symptoms of a pain disorder and/or disorder associated with pain is intractable after one or more traditional treatments used for treating the one or more symptoms and/or underlying disease or disorder.

In some embodiments, a pain disorder or a disorder associated with pain is arthritis, fibromyalgia, headache, migraine, shingles, neuropathy, failed back surgery syndrome, phantom limb pain, post traumatic pain, acute pain, pain from advanced prostate cancer, ankylosing spondylitis, AIDS-related pain, arachnoiditis, arthrofibrosis, ataxic cerebral palsy, autoimmune atrophic gastritis, autoimmune disease, avascular necrosis, back pain, Behcet's Disease/Syndrome, Breakthrough Pain, Burning Mouth Syndrome, Bursitis, CADASIL, Cancer Pain, Carpal Tunnel, Cauda Equina Syndrome, Central Pain Syndrome, Cerebral Palsy, Cerebrospinal Fluid Leak, Cervical Stenosis, Charcot-Marie-Tooth Disease, Chronic Fatigue Syndrome, Chronic Functional Abdominal Pain, Chronic Pancreatitis, Coccyx pain, collapsed lung (pneumothorax), complex regional pain syndrome (CRPS I and II), Corneal Neuropathic Pain, Crohn's disease, degenerative disc disease, Dercum's disease, dystonia, dematomyositis, diabetic peripheral neuropathy, Ehlers-Danlos syndrome, endometriosis, eosinophilia-myalgia syndrome, erythromelalgia, gout, growing pains, hydrocephalus, herniated disc, hip injury, hip replacement, knee injury, knee replacement, intercostal neuralgia, interstitial cystitis, irritable bowel syndrome, juvenile dermatositis, loin pain-haematuria syndrome, lupus, Lyme disease, medullary sponge kidney, meralgia paresthetica, mesothelioma, mitochondrial disorders, musculoskeletal pain, myofascial pain, myositis, neuropathic pain, nociceptive pain, neck pain, occipital neuralgia, osteoarthritis, Paget's disease, Parsonage Turner syndrome, pelvic pain, pinched nerve, polycystic kidney disease, polymyalgia rheumatica, polymyositis, porphyria, post-herniorraphy pain syndrome, post-mastectomy pain syndrome, post-stroke pain, post-thoracotomy pain syndrome, post-amputation stump pain, postherpetic neuralgia, post-polio syndrome, post-surgery pain, post-surgical neuralgia, post-hip surgery pain, post-traumatic stress disorder, primary lateral sclerosis, rheumatoid arthritis, psoriatic arthritis, pudendal neuralgia, radiculopathy, Raynaud's disease, restless leg syndrome, rheumatoid arthritis, sacroiliac joint dysfunction, sarcoidosis, Scheuemann's Kyphosis disease, sciatica, scoliosis, herpes zoster virus, sickle cell pain, Sjogren's syndrome, sleep apnea, spasmodic torticollis, sphincter of Oddi dysfunction, spinal cerebellar ataxia, spinal cord injury, spinal stenosis, syringomyelia, Tarlov cysts, tethered cord syndrome, thoracic outlet syndrome, temporomandibular joint syndrome, transverse myelitis, trigger point pain, ulcerative colitis, vascular pain, vasculitis, vulvodynia, whiplash, pain after hernia repair, pain after total knee replacement, pain after hip replacement, Congenital Pain Insensitivity, Anosmia, As If Personality, Borderline Personality Disorder, Malignant neoplasm of breast, Non-Small Cell Lung Carcinoma, Cold intolerance, Febrile Convulsions, Diabetes, Diabetes Mellitus, Dissociative disorder, Epilepsy, Erythromelalgia, Primary Erythermalgia, Facial Pain, Herpesviridae Infections, Hereditary Sensory Autonomic Neuropathy Type 5, Hyperplasia, Neuralgia, Hereditary Sensory and Autonomic Neuropathies, Degenerative polyarthritis, Pain, Pain in limb, Postoperative Pain, Parkinson Disease, Postherpetic neuralgia, Prostatic Neoplasms, Pruritus, Seizures, Somatoform Disorder, Tobacco Use Disorder, Trigeminal Neuralgia, Synovial Cyst, Chronic pain, Acute onset pain, Paramyotonia Congenita (disorder), Malaise, Sensory Discomfort, Burning Pain, Indifference to pain, Inflammatory pain, Mechanical pain, Scalp pain, Hereditary Motor and Sensory-Neuropathy Type II, Common Migraine, Absence of pain sensation, Malignant neoplasm of prostate, Pain Disorder, Knee Osteoarthritis, Neuropathy, Complex Regional Pain Syndromes, Tonic-clonic seizures, Inherited neuropathies, Prostate carcinoma, Breast Carcinoma, Infantile Severe Myoclonic Epilepsy, Myxoid cyst, Channelopathies, Paroxysmal Extreme Pain Disorder, Painful Neuropathy, Compressive Neuropathies, Congenital Indifference to Pain Autosomal Recessive, Generalized Epilepsy With Febrile Seizures Plus Type 2, Generalized Epilepsy With Febrile Seizures Plus 7, Febrile Seizures Familial 3B, and Small Fiber Neuropathy (Adult-onset is referred to as small fiber neuropathy), Asthma, Ataxia, Atrial Fibrillation, Hypertrophic Cardiomyopathy, Cardiovascular Diseases, Cerebellar Diseases, Dyspepsia, Experimental Autoimmune Encephalomyelitis, Primary Erythermalgia, Heart Block, Hyperalgesia, Acute Myelocytic Leukemia, Multiple Sclerosis, Neuralgia, Neuroblastoma, Pain, Postherpetic neuralgia, Tobacco Use Disorder, Trigeminal Neuralgia, Ventricular Fibrillation, Synovial Cyst, Sciatic Neuropathy, Chronic pain, Fibrillation, Hematologic Neoplasms, Electrocardiogram: P-R interval, Neuropathy, Breast Carcinoma, Central neuroblastoma, Varicella zoster, Brugada Syndrome (disorder), High grade atrioventricular block, Small Fiber Neuropathy, and Familial Episodic Pain Syndrome 2.

It is known that humans presenting with homozygous SCN9A loss-of-function mutations may suffer from congenital insensitivity to pain (CIP). In some embodiments, the SCN9A gene is involved in itching. In some embodiments, gain-of-function mutations in SCN9A and/or SCN10A cause congenital pain syndromes, such as primary erythermalgia.

Pain may occur in different parts of the body and for different reasons. For example, in some embodiments, chronic pain that occurs in most parts of the body and the extremities involve afferent neurons of the dorsal root ganglia (DRG), which reside in clusters of nerve cells near the spinal cord and have long axons extending towards, for example, the skin, muscles, and organs.

In some embodiments, a mechanism of enhanced excitability involves voltage-gated ion channels and background/leak channels that set a resting membrane potential and firing threshold of a neuron (e.g., a DRG). Generally, under conditions in which no disorder or symptom of disorder is present, chemical, mechanical, and/or thermal stimuli are required to activate receptors and ion channels in peripheral nerve endings to initiate action potentials that propagate along axons of DRG neurons. For example, in some embodiments, dendritic termini of the DRG neurons liberate neurotransmitters (e.g., glutamate and substance-P) at synapses in the spinal cord dorsal horn, activating second-order neurons that communicate pain signals to the brain. Generally (i.e., in absence of disorders or genetic changes) human DRG neurons constitutively express specific and specialized ion channels that have been implicated in afferent pain signaling. In some such embodiments, such ion channels may be targeted for modulation of one or more symptoms of a pain disorder.

In some embodiments, NaV1.7 and/or NaV1.8, are constitutively expressed in DRG neurons. In some embodiments, targeting ion channels comprising one or more proteins as described herein in accordance with the present disclosure may lead to changes (e.g., gene ablation, loss-of-function, destabilization of transcript and/or protein folding) of or in the targeted protein and/or ion channel(s). In some such embodiments, such changes in cells of a patient with one or more symptoms of a pain disorder results in reduced pain transmission and/or reduced experience of one or more pain symptoms.

Without being bound by any particular theory the present disclosure appreciates that genomic editing (e.g., disruption) of SCN9A and/or SCN10A is only desirable at a localized level. For example, nociception is essentially a protective mechanism from overextension and deformation of our joints and muscles, and is also necessary for our sense of smell. Accordingly, generalized changes in SCN9A and/or SCN10A may have undesirable consequences in addition to, e.g., amelioration and/or prevention of one or more symptoms of a pain disorder.

In some embodiments, technologies provides by the present disclosure (e.g., genome-editing agents) may be deployed (e.g., administered to or near) to DRG neurons (e.g., dysregulated DRG neurons) to modify the genes responsible for propagation of pain signals in DRG neurons.

In some embodiments, methods of the disclosure comprise treating pain in a subject by administering to a subject in need thereof a composition comprising an AAV5 vector comprising at least one sequence that encodes a guide RNA (gRNA) that targets a gene encoding a voltage-gated sodium channel subunit, wherein the gRNA comprises a spacer comprising or consisting of the nucleotide sequence of any one of SEQ ID NOs: 1-310. In some embodiments, an AAV5 vector comprises at least one gRNA sequence comprising a spacer sequence comprising the nucleotide sequence of SEQ ID NO: 5. In some embodiments, an AAV5 vector comprises at least one gRNA sequence comprising a spacer sequence comprising the nucleotide sequence of SEQ ID NO: 7. In some embodiments, an AAV5 vector comprises at least one gRNA sequence comprising a spacer sequence comprising the nucleotide sequence of SEQ ID NO: 182. In some embodiments, an AAV5 vector comprises at least one gRNA sequence comprising a spacer sequence comprising the nucleotide sequence of SEQ ID NO: 189. In some embodiments, an AAV5 vector comprises two gRNA sequences comprising spacer sequences comprising the nucleotide sequences of SEQ ID NO: 5 and 189. In some embodiments, an AAV5 vector comprises two gRNA sequences comprising spacer sequences comprising the nucleotide sequences of SEQ ID NO: 7 and 182. In some embodiments, an AAV5 vector comprises two gRNA sequences comprising spacer sequences comprising the nucleotide sequences of SEQ ID NO: 5 and 182. In some embodiments, an AAV5 vector comprises two gRNA sequences comprising spacer sequences comprising the nucleotide sequences of SEQ ID NO: 7 and 189.

In some embodiments, methods of the disclosure comprise treating pain in a subject by administering to a subject in need thereof a composition comprising an AAV9 vector comprising at least one sequence that encodes a guide RNA (gRNA) that targets a gene encoding a voltage-gated sodium channel subunit, wherein the gRNA comprises a spacer comprising or consisting of the nucleotide sequence of any one of SEQ ID NOs: 1-310. In some embodiments, an AAV9 vector comprises at least one gRNA sequence comprising a spacer sequence comprising the nucleotide sequence of SEQ ID NO: 5. In some embodiments, an AAV9 vector comprises at least one gRNA sequence comprising a spacer sequence comprising the nucleotide sequence of SEQ ID NO: 7. In some embodiments, an AAV9 vector comprises at least one gRNA sequence comprising a spacer sequence comprising the nucleotide sequence of SEQ ID NO: 182. In some embodiments, an AAV9 vector comprises at least one gRNA sequence comprising a spacer sequence comprising the nucleotide sequence of SEQ ID NO: 189. In some embodiments, an AAV9 vector comprises two gRNA sequences comprising spacer sequences comprising the nucleotide sequences of SEQ ID NO: 5 and 189. In some embodiments, an AAV9 vector comprises two gRNA sequences comprising spacer sequences comprising the nucleotide sequences of SEQ ID NO: 7 and 182. In some embodiments, an AAV9 vector comprises two gRNA sequences comprising spacer sequences comprising the nucleotide sequences of SEQ ID NO: 5 and 182. In some embodiments, an AAV9 vector comprises two gRNA sequences comprising spacer sequences comprising the nucleotide sequences of SEQ ID NO: 7 and 189.

The present disclosure also provides technologies that are superior to any current and/or traditional method of pain management or prevention. For example, in some such embodiments, strategies for treatment or prevention of pain (e.g., pain suppression) described herein are superior to traditional methods of pain management due to their high specificity, efficacy, and safety profile.

In some embodiments, technologies provided by the present disclosure may be combined with one or more current or traditional methods of pain management.

In some embodiments, pain is evaluated by one of skill in the art and/or by a subject experiencing pain. In some such embodiments, pain is evaluated relative to pain experienced before, during, and/or after treatment. In some embodiments, pain is evaluated between subjects, such as between subjects receiving a placebo or control treatment and subject receiving a treatment that comprises one or more genetic modifications to, e.g., SCN9A and/or SCN10A.

In some embodiments, a level or measure of pain reveals reduction, amelioration, prevention, or attenuation in one or more symptoms of pain or a disorder associated with pain, relative to a control. In some such embodiments, a control may be within a single subject (e.g., experience prior to administration of a composition of the present disclosure) or as compared to a control subject (e.g., a subject receiving a placebo or control composition).

By way of non-limiting example, in some embodiments, pain may be measured based on average level of pain over a period of time, relative to average level of pain over a period of time that occurred prior to treatment in accordance with the present disclosure.

In some embodiments, pain is evaluated based on one or more measures of efficacy as described in the present disclosure.

In some embodiments, various tools and measurements of pain will be known to one of skill in the art.

The disclosure is further illustrated by the following example. This example is provided for illustrative purposes only. It is not to be construed as limiting the scope or content of the disclosure in any way.

### EXAMPLES

### Example 1—gRNA Candidate Screen

In the present example, spacer sequences targeting the genes SCN9A and SCN10A respectively, whose products function in tandem to transmit “painful” stimuli, were screened for cleavage efficacy and indel formation frequency.

Ribonucleoproteins (RNPs) were complexed from bacterially expressed and purified protein and chemically synthesized RNA oligonucleotides. crRNAs and tracrRNA for SaCas were made as RNA oligomers using standard oligonucleotide synthesis (made by IDT vendow). RNA molecules were desalted and lyophilized. crRNAs and tracrRNAs were resuspended in H150 buffer (10 mM HEPES, N-2-hydroxyethylpiperazine-N′-2-ethanesulfonic acid, 150 mM NaCl, pH 7.5) to a concentration of 333 μM and then mixed at a 1:1 ratio. The crRNA and tracrRNA were then heat annealed at 95° C. for 5 minutes and slow cooled to form the full-length gRNA at 200 μM concentration. SaCas9 protein was made by Aldevron, concentrated to 0.4 mM, buffer exchanged to HG300 buffer (50 mM HEPES, 300 mM NaCl, 1 mM TCEP, 20% glycerol (% v/v), pH 7.5), aliquoted, and stored at −80° C. Annealed guides were mixed with 110 μM SaCas9 protein in H300 buffer (50 mM HEPES, 300 mM NaC, 1 mM tris(2-carboxyethyl)phosphine, 20% glycerol (% v/v), pH 7.5) to make a final 55 μM RNP. RNP was then frozen at −80° C. until ready for use.

Human T cells were nucleofected with the RNP, grown for 4 days, and then genomic DNA (gDNA) collected for Next Generation Sequencing (NGS). Primary human CD4+T-cells were isolated from whole blood samples (Massachusetts General Hospital, HemaCare) using Miltenyi CD4 Microbeads (Catalog number 130-045-101). Cells were frozen down once isolated in Cryotstor CS10 cell freezing medium (STEMCELL, Catalog #: 07930) and stored in liquid nitrogen for future use. 250,000 cells were thawed, pelleted, resuspended in buffer P2 (Lonza), and aliquoted into 96 well nucleofection plates at 20 μl per well. 2 μl of individual test RNPs (as described above) were added to each well. The cells were nucleofected using the Lonza 4D-Nucleofector (AAF-1002B 4D-Nucleofector Core unit, 96-well Shuttle™ Device) using the CA-137 program at an RNP dose of 5 μM. After nucleofection, the cells were incubated at room temperature for 5-10 min. Then 30 μl of complete expansion medium (see Media Components Table (Table 6) for additional details) was added, and the cell mix transferred to a 96-well plate containing 100 μl pre-warmed complete expansion medium. The cell plates were incubated at 37° C. 5% CO2 and collected 96 hours post nucleofection. Genomic DNA was isolated using the Agencourt DNAdvance kit (Beckman Coulter) according to manufacturer's instructions and quantified using Qubit fluorometric quantitation (ThermoFisher).

For Illumina amplicon sequencing, two rounds of amplification were performed: round 1 targets the off-target region, and round 2 adds the full-length Illumina adapter sequence. Round 1 was performed in a 12 μL reaction volume, consisting of 6 μL of NEBNext® Ultra™ II Q5@ Master Mix (New England Biolabs), 0.5 μM total primers (0.25 μM forward and 0.25 μM reverse), nuclease free water, and 20 ng of gDNA template. PCR conditions were as follows: 30 s at 98° C. for initial denaturation, followed by 20 cycles of 10s at 98° C. for denaturation, 15 s at 60° C. for annealing, 30s at 72° C. for extension, and 5 min at 72° C. for the final extension. Four μl of round 1 PCR product was added directly into the 12 μl round 2 reaction volume, consisting of 6 μL of NEBNext® Ultra™ II Q5@ Master Mix (New England Biolabs), 2 μM total primers (1 μM forward and 1 μM reverse), and nuclease free water. PCR conditions were as follows: 30 s at 98° C. for initial denaturation, followed by 14 cycles of 10s at 98° C. for denaturation, 15 s at 60° C. for annealing, 30s at 72° C. for extension, and 5 min at 72° C. for the final extension. Equal volumes of each PCR product was pooled and purified using (0.9×) Agencourt AMPure XP beads (Beckman Coulter Agencourt AMPure XP—PCR Purification #A63882) as per the manufacturer's protocol followed by size 300-1200 bp size selection on the BluePippin (Sage Science, Beverly, MA) and loaded on the Illumina MiSeq with 10-20% phiX.

Analysis of indel rates at the predicted cut site (e.g., editing rates) was done as described in Bothmer, et. al, Nat. Communications, 8, 13905 (2017), but reads were aligned to reference sequences using bowtie2, version 2.1.0, instead of needle as in Bothmer et al. Table 4 shows “tiers” of values for measurements of average indel fraction windows and results of the analysis described herein are shown in Table 5.

TABLE 4
Tiers of Average Indel Fraction Window Measurements

| Tier | Value (%) |
|---|---|
| A | 80-100 |
| B | 60-80 |
| C | 40-60 |
| D | 20-40 |
| E | 0.02-20 |

TABLE 5
Average Indel Fraction Window Measurements

| | Avg. Indel |
|---|---|
| Spacer | Fraction |
| SEQ ID | Window |
| 1 | C |
| 2 | C |
| 3 | D |
| 4 | B |
| 5 | A |
| 6 | D |
| 7 | B |
| 8 | C |
| 9 | D |
| 10 | D |
| 11 | B |
| 12 | E |
| 13 | E |
| 14 | E |
| 15 | E |
| 16 | D |
| 17 | E |
| 18 | B |
| 19 | E |
| 20 | A |
| 21 | C |
| 22 | E |
| 23 | E |
| 24 | E |
| 25 | E |
| 26 | E |
| 27 | E |
| 28 | E |
| 29 | E |
| 30 | E |
| 31 | E |
| 32 | E |
| 33 | E |
| 34 | E |
| 35 | C |
| 36 | E |
| 37 | E |
| 38 | E |
| 39 | E |
| 40 | E |
| 41 | E |
| 42 | E |
| 43 | E |
| 44 | E |
| 45 | E |
| 46 | E |
| 47 | E |
| 48 | C |
| 49 | E |
| 50 | E |
| 51 | E |
| 52 | E |
| 53 | E |
| 54 | E |
| 55 | E |
| 56 | E |
| 57 | E |
| 58 | E |
| 59 | E |
| 60 | E |
| 61 | E |
| 62 | E |
| 63 | E |
| 64 | E |
| 65 | E |
| 66 | E |
| 67 | E |
| 68 | E |
| 69 | D |
| 70 | B |
| 71 | D |
| 72 | A |
| 73 | E |
| 74 | E |
| 75 | E |
| 76 | E |
| 77 | E |
| 78 | E |
| 79 | E |
| 80 | E |
| 81 | E |
| 82 | E |
| 83 | E |
| 84 | E |
| 85 | E |
| 86 | D |
| 87 | E |
| 88 | E |
| 89 | E |
| 90 | E |
| 91 | E |
| 92 | E |
| 93 | E |
| 94 | E |
| 95 | E |
| 96 | E |
| 97 | E |
| 98 | E |
| 99 | E |
| 100 | B |
| 101 | E |
| 102 | E |
| 103 | E |
| 104 | E |
| 105 | E |
| 106 | C |
| 107 | E |
| 108 | E |
| 109 | E |
| 110 | E |
| 111 | E |
| 112 | E |
| 113 | E |
| 114 | E |
| 115 | E |
| 116 | E |
| 117 | E |
| 118 | E |
| 119 | E |
| 120 | E |
| 121 | E |
| 122 | E |
| 123 | E |
| 124 | E |
| 125 | D |
| 126 | E |
| 127 | E |
| 128 | E |
| 129 | D |
| 130 | E |
| 131 | E |
| 132 | E |
| 133 | E |
| 134 | D |
| 135 | E |
| 136 | E |
| 137 | E |
| 138 | E |
| 139 | E |
| 140 | E |
| 141 | E |
| 142 | E |
| 143 | E |
| 144 | E |
| 145 | E |
| 146 | E |
| 147 | E |
| 148 | E |
| 149 | E |
| 150 | E |
| 151 | E |
| 152 | E |
| 153 | E |
| 154 | E |
| 155 | B |
| 156 | E |
| 157 | E |
| 158 | E |
| 159 | E |
| 160 | E |
| 161 | E |
| 162 | E |
| 163 | E |
| 164 | E |
| 165 | E |
| 166 | E |
| 167 | E |
| 168 | E |
| 169 | E |
| 170 | E |
| 171 | E |
| 172 | E |
| 173 | D |
| 174 | E |
| 175 | E |
| 176 | E |
| 177 | D |
| 178 | D |
| 179 | C |
| 180 | C |
| 181 | C |
| 182 | C |
| 183 | C |
| 184 | D |
| 185 | B |
| 186 | D |
| 187 | C |
| 188 | B |
| 189 | C |
| 190 | C |
| 191 | E |
| 192 | E |
| 193 | D |
| 194 | E |
| 195 | E |
| 196 | D |
| 197 | E |
| 198 | E |
| 199 | E |
| 200 | E |
| 201 | C |
| 202 | E |
| 203 | E |
| 204 | C |
| 205 | C |
| 206 | E |
| 207 | E |
| 208 | E |
| 209 | E |
| 210 | E |
| 211 | E |
| 212 | E |
| 213 | E |
| 214 | E |
| 215 | E |
| 216 | E |
| 217 | E |
| 218 | E |
| 219 | E |
| 220 | E |
| 221 | E |
| 222 | E |
| 223 | C |
| 224 | E |
| 225 | E |
| 226 | E |
| 227 | E |
| 228 | E |
| 229 | E |
| 230 | E |
| 231 | E |
| 232 | D |
| 233 | E |
| 234 | E |
| 235 | E |
| 236 | C |
| 237 | E |
| 238 | E |
| 239 | E |
| 240 | D |
| 241 | E |
| 242 | E |
| 243 | E |
| 244 | E |
| 245 | B |
| 246 | E |
| 247 | E |
| 248 | E |
| 249 | E |
| 250 | E |
| 251 | E |
| 252 | E |
| 253 | E |
| 254 | E |
| 255 | E |
| 256 | E |
| 257 | E |
| 258 | E |
| 259 | E |
| 260 | E |
| 261 | E |
| 262 | E |
| 263 | E |
| 264 | E |
| 265 | C |
| 266 | E |
| 267 | E |
| 268 | E |
| 269 | E |
| 270 | E |
| 271 | E |
| 272 | E |
| 273 | E |
| 274 | E |
| 275 | C |
| 276 | E |
| 277 | E |
| 278 | E |
| 279 | E |
| 280 | E |
| 281 | E |
| 282 | E |
| 283 | B |
| 284 | D |
| 285 | C |
| 286 | E |
| 287 | E |
| 288 | E |
| 289 | D |
| 290 | E |
| 291 | E |
| 292 | E |
| 293 | E |
| 294 | C |
| 295 | E |
| 296 | E |
| 297 | E |
| 298 | E |
| 299 | E |
| 300 | E |
| 301 | E |
| 302 | E |
| 303 | E |
| 304 | E |
| 305 | E |
| 306 | E |
| 307 | E |
| 308 | E |
| 309 | E |
| 310 | E |

TABLE 6
Media Components

| Item | Vendor | | Catalog |
|---|---|---|---|
| x-vivo 15 media | Lonza | | BE02-054Q |
| human ab serum | Gemini | | 100-512 |
| | bioproducts | | |
| N-Acetyl-L-cysteine | Sigma Aldrich | | A9165-25G |
| CTS ™ GlutaMAX ™-I Supplement | Thermo fisher | | A1286001 |
| Dynabeads ™ Human T-Activator | Thermo fisher | | 11132D |
| CD3/CD28 for T Cell Expansion and | | | |
| Activation | | | |
| Recombinant Human IL-2 | Peprotech | | 200-02 50 ug |
| Recombinant Human IL-7 10 ug | Peprotech | | 200-07 10 ug |
| Recombinant Murine IL-15 10 ug | Peprotech | | 210-15 10 ug |
| | Final | | |
| | Concentration | | Example |
| T cell media: | | | 50 mL |
| X-Vivo 15 Media | | | 46.2 |
| FBS Serum (heat inactivated) | 5% | | 2.5 |
| N-acetylcysteine (100 mg/mL) | 1.6 | mg/mL | 0.8 |
| L-alany1-L-glutamine (GlutaMax) | 2 | mM | 0.5 |
| (200 mM) | | | |
| T cell stimulation media: | | | |
| Sterile filtered T cell media | | | 50 mL |
| hIL-2 (stock: 100,000 IU/mL) | 100 | IU/mL | 0.05 |
| rhIL-7 (stock: 5 ug/mL) | 10 | ng/ml | 0.1 |
| rhIL-15(stock: 5 ug/mL) | 10 | ng/ml | 0.1 |
| T cell expansion media: | | | |
| Sterile filtered T cell media | | | 50 mL |
| hIL-2 (stock: 100,000 IU/mL) | 50 | IU/mL | 0.025 |
| rhIL-7 (stock: 5 ug/mL) | 5 | ng/mL | 0.05 |
| rhIL-15 (stock: 5 ug/mL) | 0.5 | ng/ml | 0.005 |

### Example 2—AAV Serotype Candidate Screen

In the present example, recombinant AAV (rAAV) viral particles comprising constructs as described herein were produced and transduction efficiencies and transcriptional intensities in human explant Dorsal Root Ganglion (DRG) neurons, nociceptor, and non-neuronal cell types were determined.

rAAV Particles

rAAV particles of the AAV5, and AAV9 serotypes, which included a CMV driven eGFP encoding nucleotide sequence, were obtained from Asklepios BioPharmaceutical, Inc. and/or SIRION Biotech International Inc.

### Production and Transduction of DRG Explants

Approximately 75 DRG explants were created from human donor DRGs and were randomized across test conditions. Approximately 24 hr after explants were generated, all media was removed from the explants and fresh DRG media containing rAAV at 3×10⁹, 3×10¹⁰, or 3×10¹¹ VG/ml was added to each explant, and the plate containing explants was returned to a 37° C. incubator. Approximately 24 hr after infection, 300 μl of fresh DRG media was added to each well. Approximately 72 hr after infection, a half media change was performed with fresh DRG media (300 μl media removed, 300 μl of media added). Additional half media changes were performed three times per week until explants were processed for histology and/or RNA isolation. Alternatively, for bloody explants, initial transduction events were performed 96 hr after explant generation to facilitate an additional media change prior to transduction to wash out the blood. In general, transduction typically occurred 24-48 hr following sectioning.

Explants were cultured for four weeks before formalin-fixed paraffin-embedding (FFPE) fixation using standard methods known in the art. In brief, 1) explants were transferred to 1.7 ml microcentrifuge tubes containing 1 ml 4% PFA in PBS; 2) explants were incubated for 1 hr at room temperature; 3) supernatant was removed and explants were washed with 1 ml PBS for 1 min; 4) supernatant was removed and 1 ml 25% ethanol was added, samples were incubated for 30 minutes; 5) supernatant was removed, and 1 ml 50% ethanol was added, samples were incubated for 30 minutes; 6) supernatant was removed, and 1 ml 70% ethanol was added, samples were incubated for 30 minutes; if sample solution was cloudy, the supernatant was replaced with a new aliquot of 70% ethanol; 7) samples were stored at 4C until additional processing; 8) samples were embedded in paraffin, followed by 5 μm sectioning and slide creation in preparation for histological analysis.

### Imaging of Fixed DRG Explant Sections

Sections were stained with an anti-TUJ1 (neuron-specific class III P-tubulin) antibody and an anti-peripherin (class-III neuronal intermediate filament protein) antibody. Three slides were created for each explant, with a total of 24 sections per condition, leading to approximately 200 slides for detection and quantification of neuronal and/or nociceptor transduction efficiency and translational intensity determination.

### Results

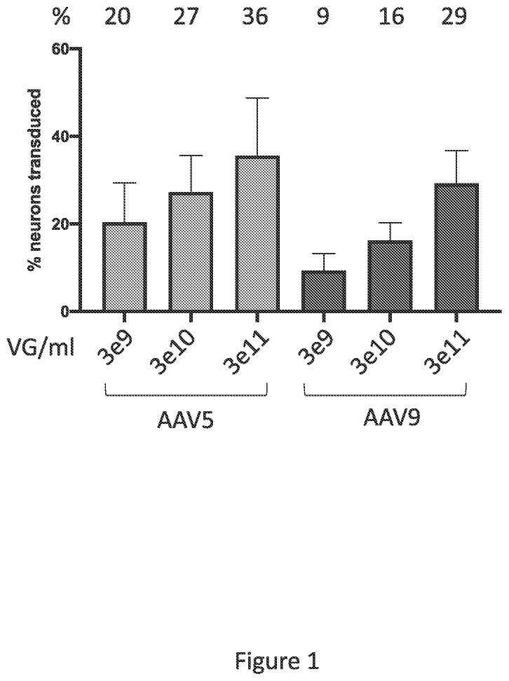

FIG. 1 depicts the neuron cell transduction efficiency for the noted rAAV serotypes at the specified concentrations. The results show that each of the rAAV serotypes transduced neurons in a dose-dependent manner. As shown in FIG. 1, AAV5 transduced 20%, 27%, and 36% of the neurons at 3×10⁹, 3×10¹⁰, 3×10¹¹ VG/ml, respectively, and AAV9 transduced 9%, 16%, and 29% of the neurons at 3×10⁹, 3×10¹⁰, 3×10¹¹ VG/ml, respectively.

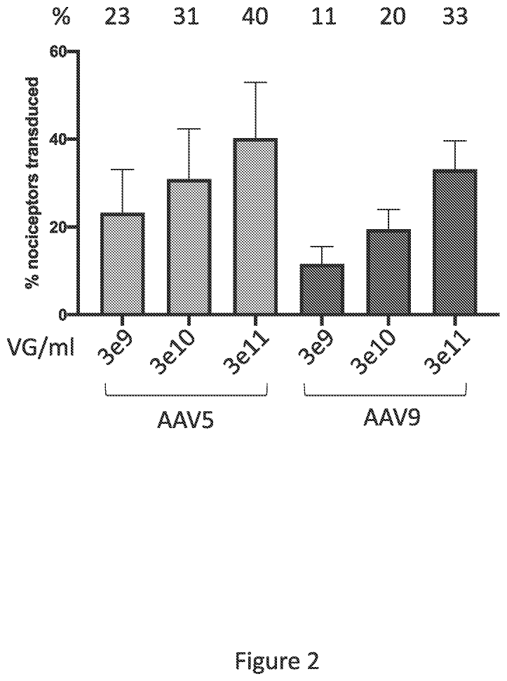

FIG. 2 depicts the nociceptor cell transduction efficiency for the noted rAAV serotypes at the specified concentrations. The results show that each of the rAAVs transduced nociceptors in a dose-dependent manner. As shown in FIG. 2, AAV5 transduced 23%, 31%, and 41% of the nociceptors at 3×10⁹, 3×10¹⁰, 3×10¹¹ VG/ml, respectively, and AAV9 transduced 11%, 20%, and 33% of the nociceptors at 3×10⁹, 3×10¹⁰, 3×10¹¹ VG/ml, respectively.

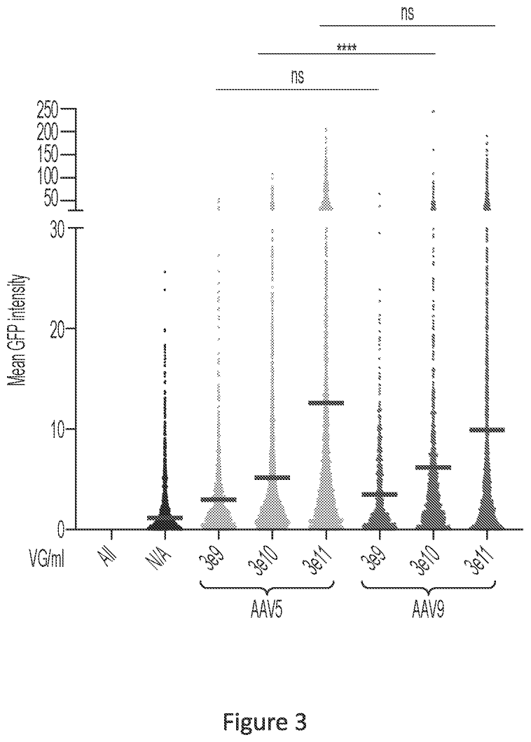

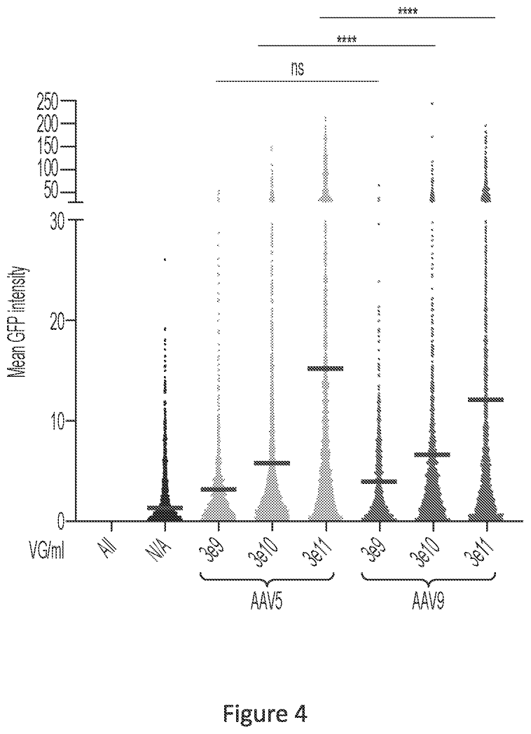

FIG. 3 depicts the mean GFP intensity in transduced neurons, a proxy for translational intensity following cellular transduction. FIG. 4 depicts the mean GFP intensity in transduced nociceptors, a proxy for translational intensity following cellular transduction. The data from FIGS. 3 and 4 show that each of the rAAVs tested induced higher mean GFP intensities in transduced neurons and transduced nociceptors than the GFP intensity produced by a control sample that was not infected with an rAAV (N/A in FIGS. 3 and 4).

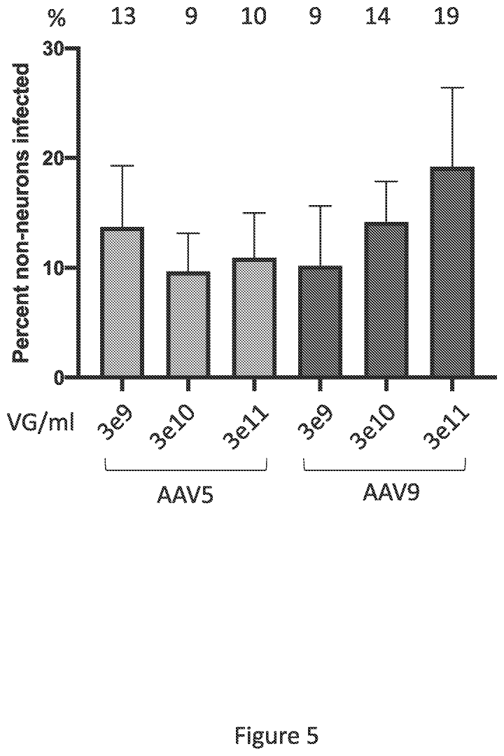

FIG. 5 depicts the average percentage of non-neuronal cell rAAV transduction. As shown in FIG. 5, AAV5 transduced 13%, 9%, and 10% of non-neuronal cells at 3×10⁹, 3×10¹⁰, 3×10¹¹ VG/ml, respectively, and AAV9 transduced 9%, 14%, and 19% of non-neuronal cells at 3×10⁹, 3×10¹⁰, 3×10¹¹ VG/ml, respectively. Together, these results demonstrate successful infection of DRG explants with rAAV constructs, leading to preferential efficient transduction of neuronal and nociceptor cells.

### Example 3—Ex-Vivo Intra-DRG Injection of AAV Constructs

In the present example, parameters for ex-vivo intra-DRG injection of AAV constructs into human and Rhesus macaque whole DRG were assessed.

### Whole Intra-DRG Injections in Human DRGs

Whole human donor DRG segments (left and right for L3 through S2) were obtained from vendor Donor NetWork West, at a postmortem interval of <6-8 hr, with a total time from extraction to dissection of <18 hr. DRG segments were cultured and subjected to ex-vivo injection of AAV5/9-CMV-eGFP (3×10¹² VG/ml) in 1×PBS buffer with 0.014% tween20 and 0.1% Evans Blue dye. Injection rates varied from ˜0.5 μl/min to ˜2.5 μl/min, with grossly non-physiologically damaging injection volumes ranging from ˜10 μl to ˜50 μl.

### Whole Intra-DRG (Cauda Equine) Injections in Rhesus Macaque

Whole NHP (Rhesus macaque) DRG segments (Cauda equine) were subjected to ex-vivo injection of vehicle 1×PBS buffer with 0.014% tween20 and 0.1% Evans Blue dye. Injection rates varied from ˜0.5 μl/min to ˜2.0 μl/min, with grossly non-physiologically damaging injection volumes ranging from ˜10 μl to ˜20 μl.

### Results

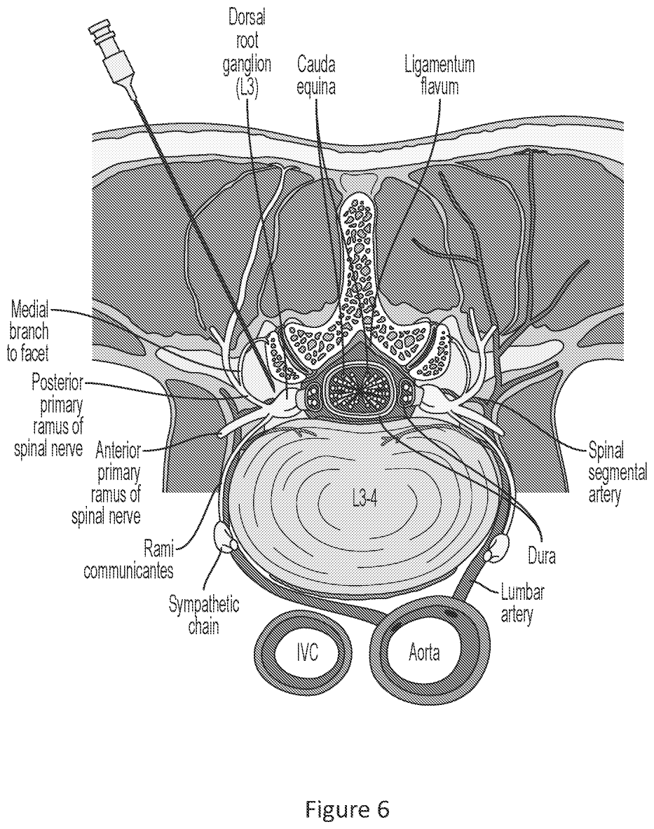

The series of ex-vivo injected human DRG segments revealed an optimal injection volume of ˜45 μl administered at ˜1 μl/min when delivered to the human DRG left and/or right L4 or L5 segment. Additionally, for NHP DRG injections, the optimal volume to be administered was revealed as ˜10 μl delivered at ˜1 μl/min. These results suggest that intra-DRG in-vivo injection between the L3-L4 segment is feasible (as depicted in FIG. 6).

### Example 4—Determining Exemplary rAAV-gRNA Based Editing Efficiencies

In the present example, the editing efficacy of guide RNA sequences for endonuclease induced SCN9A and SCN10A gene modification were determined utilizing rAAV particles comprising constructs as described herein.

### Dose Response Studies in DRG Explants

rAAV particles of the AAV5 serotype comprising CMV driven Cas9-encoding nucleotide sequence and either nucleotide sequences encoding gRNAs of SEQ ID NOs: 5 and 189 (“VIR-A”, targeting SCN9A and SCN10A, respectively), or nucleotide sequences encoding gRNAs of SEQ ID NOs: 7 and 182 (“VIR-B”, targeting SCN9A and SCN10A, respectively), were produced using standard methods known in the art. DRG explants were produced from seven human donor DRGs. Approximately 209 samples were created, each sample comprising two explants. DRG explants were randomized across test conditions. Approximately 24 hours following explant production, samples were infected with rAAV vectors. Explants were infected using 300 μl of media with a final concentration of about 3×10⁹, 3×10¹⁰, or 3×10¹¹ VG/ml. Explants were maintained as described above for four weeks before DNA and RNA harvesting for analysis using ddPCR and sequencing, targeted RNA sequencing, drop of RTddPCR, and NMD RTddPCR.

### RNA Analysis—RNA Sequencing, Drop-Off RTddPCR, NMD RTddPCR

TABLE 7
equipment for Drop-off RTddPCR, NMD RTddPCR

| | | Catalog |
|---|---|---|
| Equipment | Vendor | Number |
| AutoDG | BioRad | 1864101 |
| QX200 Droplet Reader | BioRad | 1864003 |
| PX1 Plate Sealer | BioRad | 1814000 |
| Veriti 96-well thermal cycler | Applied Biosystems | 4375786 |

TABLE 8
equipment for Drop-off RTddPCR, NMD RTddPCR

| | | Catalog |
|---|---|---|
| Material/Reagent | Vendor | Number |
| BioRad ddPCR 96-well Plate | BioRad | 12001925 |
| Pipet Tips for AutoDG System | BioRad | 1864121 |
| BioRad Pierceable Foil Heat Plate Seals | BioRad | 1814040 |
| One-step RT-ddPCR Advanced Kit for Probes | BioRad | 1864022 |
| DG32 AutoDG Cartridges | BioRad | 1864109 |
| AutoDG Droplet Generation Oil | BioRad | 1864110 |
| Pipette tips for AutoDG | BioRad | 1864120 |
| ddPCR Droplet Reader Oil | BioRad | 1863004 |

Premixed PrimeTime qPCR assays were ordered from Integrated DNA technologies Inc. (IDT). Assays were custom ordered for 3.6:1 primer:probe ratios, and were supplied at 20×. A total of 350ng RNA per sample was utilized for quantification. qPCR reaction mixtures were created according to manufacturer specifications. TRPV1 was utilized as an internal control for qPCR amplification normalization. Droplets were generated using AutoDG prior to combined RT-PCR and amplification PCR conducted in Veriti thermocyclers using standard methods. Following amplification, plates were loaded into a QX200 droplet reader and quantification was performed using the QuantSoft™ software. RNA sequencing was performed using standard methods.

### Results

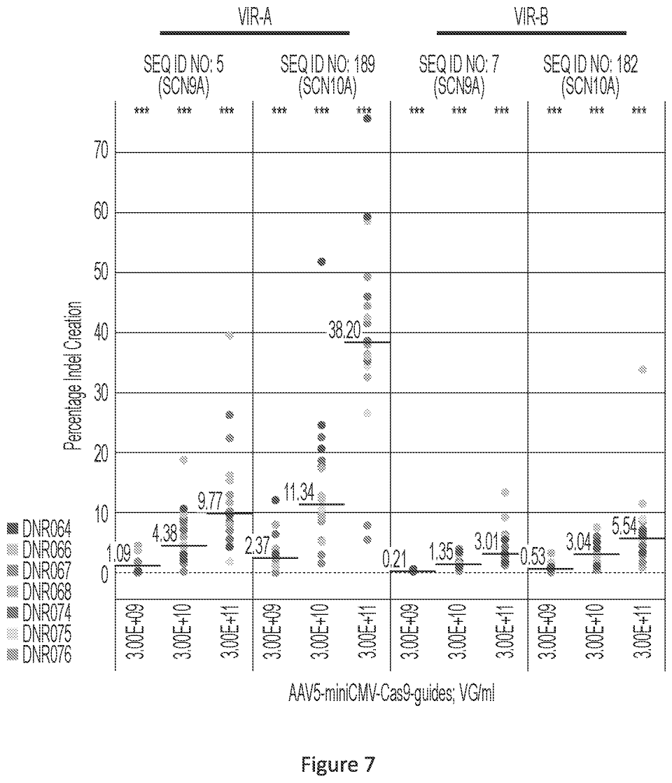

As depicted in FIG. 7, drop-off RT-ddPCR revealed that VIR-A (encoding gRNAs of SEQ ID NO: 5 and SEQ ID NO:189) and VIR-B (encoding gRNAs of SEQ ID NO: 7 and SEQ ID NO:182) exhibited human DRG explant DNA editing at the SCN9A and SCN10A loci. As shown in FIG. 7, VIR-A exhibited median indel creation of 1.09%, 4.38%, and 9.77% for SCN9A, and of 2.37%, 11.34%, and 38.2% for SCN10A, at 3×10⁹, 3×10¹⁰, 3×10¹¹ VG/ml, respectively. VIR-B exhibited median indel creation of 0.21%, 1.35%, and 3.01% for SCN9A, and of 0.53%, 3.04%, and 5.54% for SCN10A, at 3×10⁹, 3×10¹⁰, 3×10¹¹ VG/ml, respectively.

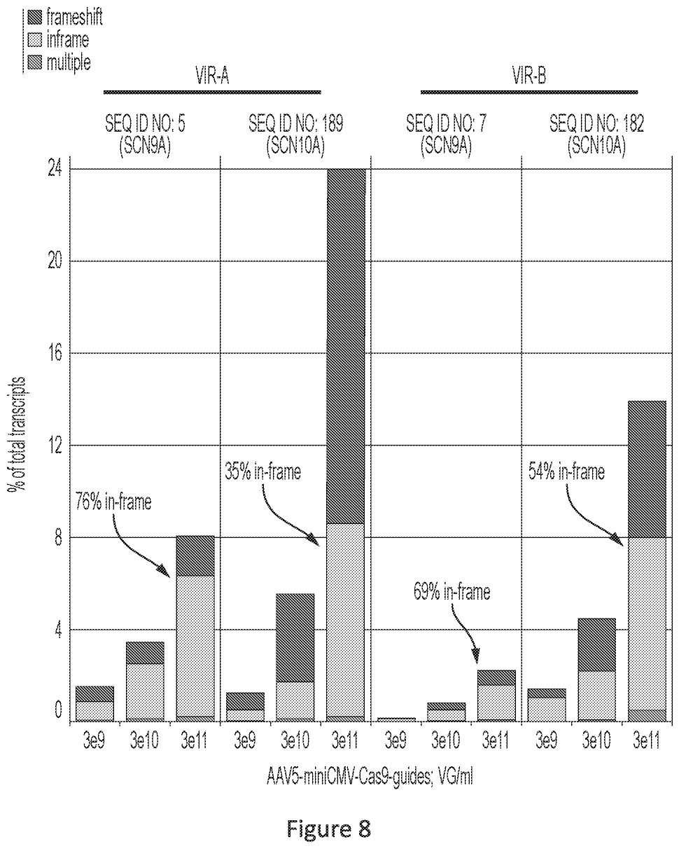

FIG. 8 depicts type of indels generated as determined by targeted RNA sequencing. As shown in FIG. 8, when infected at 3×10¹¹ VG/ml, VIR-A resulted in indels in about 8% of total transcripts for SCN9A (76% of which were in-frame indels), and resulted in indels in about 24% of total transcripts for SCN10A (35% of which were in-frame indels). When VIR-B was infected at 3×10¹¹ VG/ml, VIR-B resulted in indels in about 2% of total transcripts for SCN9A (69% of which were in-frame indels), and resulted in indels in about 15% of total transcripts for SCN10A (54% of which were in-frame indels).

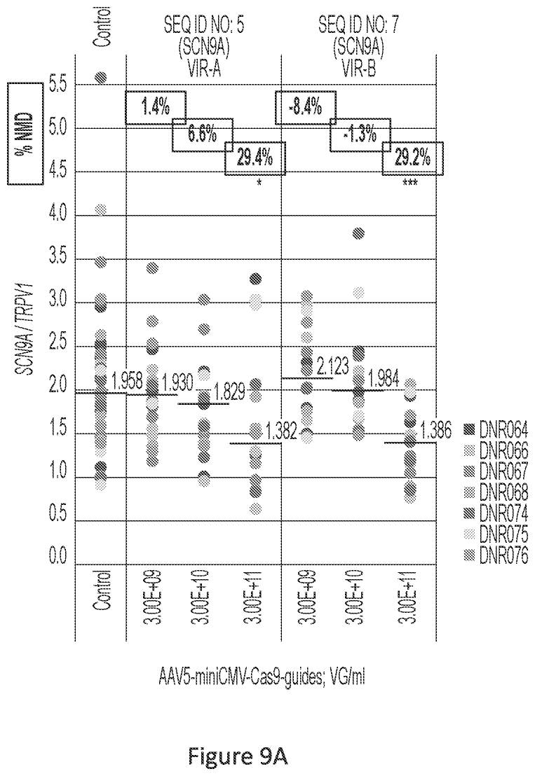

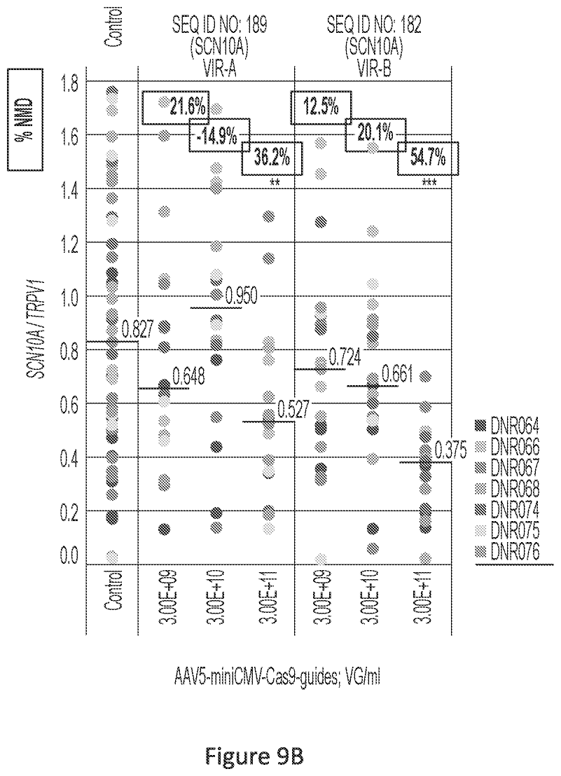

FIGS. 9A and 9B depict expression of SCN9A or SCN10A relative to TRPV1, as determined by RT-ddPCR, as well as percentage of transcripts lost to nonsense mediated decay. As shown in FIG. 9A, when infected at 3×10¹¹ VG/ml, VIR-A and VIR-B resulted in SCN9A transcripts that were lost at a rate of 29.4% and 29.2%, respectively. As depicted in FIG. 9B, when infected at 3×10¹¹ VG/ml, VIR-A and VIR-B resulted in SCN10A transcripts that were lost at a rate of 36.2% and 54.7%, respectively.

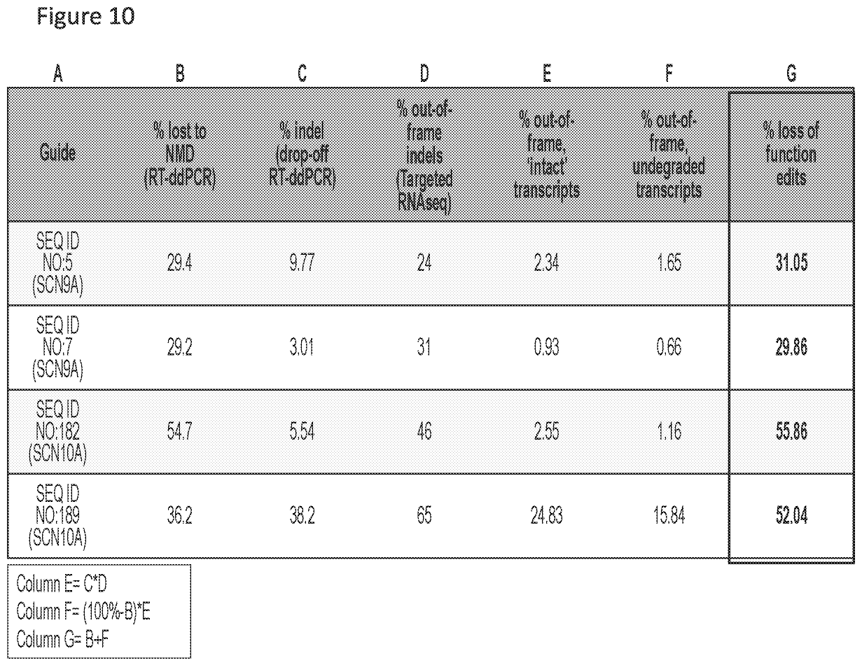

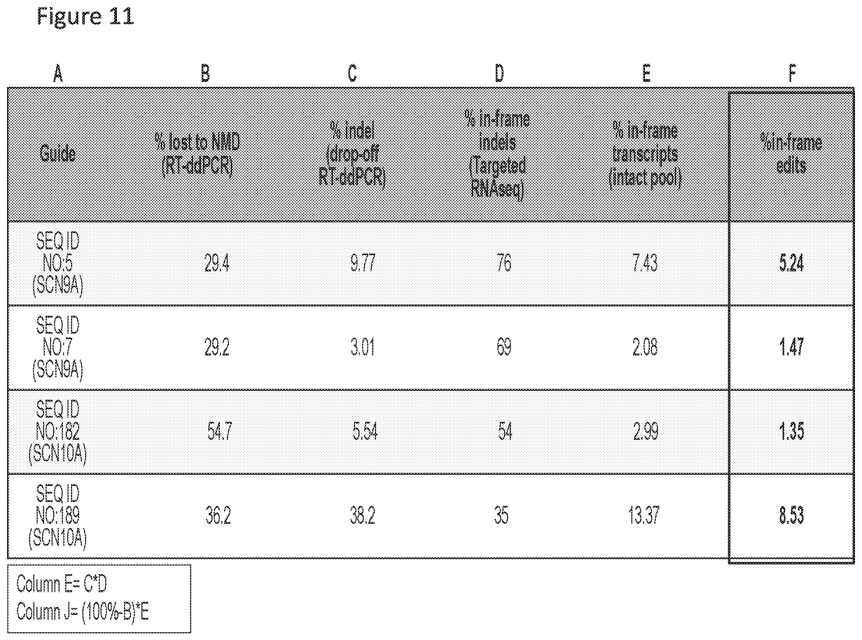

Depicted in FIGS. 10 and 11 are summaries of mutation rates and the total percentage of presumed loss of function edits in human DRG explants treated with AAV5-CMV-Cas9-guide at 3×10¹¹ VG/ml.

### Example 5—In-Vivo Muridae Genome Editing

In the present example, molecular and physiological phenotype changes induced by delivery of rAAV constructs are determined in-vivo in rat models. rAAV particles of the AAV5 serotype and comprising CMV driven Cas9 and GFP nucleotide sequences are produced using standard methods known in the art. Additional rAAV constructs include CMV driven Cas9 nucleotide sequence and nucleotide sequences encoding gRNAs targeting SCN9A and SCN10A as described herein.

### Injection Parameters and Longitudinal Behavioral Assays

Adult animals undergo 10p intrathecal injection of rAAV particles (3×10¹⁰VG/ml). At day 35 post-injection, each animal's general physiological well-being and coordination are analyzed using the rotarod assessment. At day 41 post-injection, animals undergo surgical incision (brennan model). At day 42 post-injection, animals undergo behavioral analysis (tactile allodynia—Electronic von Frey Test; tactile hyperalgesia—Pinchmeter Test (Randall-Selitto); and thermal hyperalgesia—plantar test (Hargreaves)).

### Molecular Assessments on DRGs (L3-L6)

Following the live portion of the experiment, animals are euthanized and DRG segments (L3-L6) are harvested for molecular characterization. Editing percentages are determined utilizing RT-ddPCR; nonsense-mediated-decay (NMD) levels are determined utilizing RT-ddPCR; and alternative NaV channel transcriptional compensation is determined utilizing RT-qPCR.

### Results

Treatment with VIR particles comprising nucleotide sequences encoding gRNAs targeting SCN9A and SCN10A as described herein result in alterations to the tactile allodynia response in animals injected with test AAV particles when compared to vehicle or control AAV, Treatment with VIR particles comprising nucleotide sequences encoding gRNAs targeting SCN9A and SCN10A as described herein result in increases in pain threshold at 6-weeks post injection when compared to vehicle or control AAV injection, as determined by One-way ANOVA with Dunnett's/Tukey's multiple comparison test. Treatment with VIR particles comprising nucleotide sequences encoding gRNAs targeting SCN9A and SCN10A as described herein result in murine tactile hyperalgesia assay alterations (pain in response to a robust stimuli) when compared to vehicle or control AAV injection as determined using the pinchmeter test (Randall-Selitto).

### Example 6—In-Vivo Cynomolgus DRG Delivered Cas9 Toxicity and Genome Editing

In the present example, molecular and physiological phenotype changes induced by delivery of rAAV particles comprising constructs as described herein are determined in-vivo in non-human primate (NHP) models.

### Injection Parameters and Longitudinal Study Design

Prior to injection, adult Cynomolgus animals undergo functional observational batteries (FOBs) to identify baseline physiological characteristics. FOBs are performed prior to, during, and at the immediate terminus of the live portion of the experiment and include but are not limited to assays to determine: body temperature, reflexes (Chaddock and Baniski), movement, posture, gait, proprioception, paresis, ataxia, dysmetria, slope assessment and/or nerve conduction velocity.

Adult Cynomolgus animals are injected with a single-unilateral intra-DRG injection at the L4 and/or L5 segment as depicted diagrammatically in FIG. 6. A suitable injection volume (e.g., approximately 10 μl to 50p1) comprising AAV5 or AAV9 serotypes comprising miniCMV-Cas9 constructs are injected. Two animals for each condition are utilized, with an additional two animals injected with vehicle as control.

Identifying AAV with Ideal Tropism and Biodistribution Characteristics In-Vivo

At the termination of the living stage of the assay, necroscopy is performed to facilitate molecular characterization of tissues of interest. Tissues such as the DRGs (left and right for L4, L5, and S1) spinal cord (L1-L5), and spinal nerve root (L5) are collected and samples are flash frozen and stored at −80° C. for non-histological characterization, and/or fixed and stored for later histological characterization using standard methodologies as are known in the art (e.g., samples are fixed in approximately 4% PFA or approximately 10% formalin at 4° C. for 24 hrs, and are then transferred to 70% EtOH for storage at ambient temperature). Additional tissues of interest (e.g., thoracic segments T1-12, and sacral segments S1-S5, liver biopsies, heart biopsies, brain stem biopsies, brain biopsies, peripheral nerve bundles L4-L5 left/right, and/or dermatome biopsies L4-L5 left/right) are flash-frozen using liquid nitrogen and retained at −80° C. for non-histological characterization.

Fixed tissues are histologically analyzed using various staining assays as known in the art. For example, histological assays may comprise but are not limited to: Cas9 Immunohistochemistry to determine AAV tropism within DRGs in-vivo, Haemotoxylin and Eosin (H&E) staining to detect morphological damage, Neuron-specific class III beta-tubulin (Tuj 1) and Neuronal nuclei antigen (NeuN, also known as RNA binding protein fox-1 homolog 3) staining to determine neuronal cell body toxicity, Platelet endothelial cell adhesion molecule (PECAMI, also known as CD31) staining to determine and detect damage to vascular morphology, density, and/or distribution, Cleaved Caspase 3 (CASP3) staining to detect cell death and/or stress, Ionized calcium binding adapter molecule 1 (iba1) staining to determine and detect activated microglia, T-cell surface glycoprotein CD3 epsilon (CD3) staining to characterize mononuclear cells of the T-lineage, and/or B-lymphocyte antigen CD20 (CD20, also known as MS4A1) staining to characterize mononuclear cells of the B-lineage.

Frozen tissue samples are characterized by DNA and RNA isolation followed by AAV biodistribution analysis utilizing Cas9 mRNA presence as measured by qPCR as a proxy.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

LISTING OF SEQUENCES
1. SCN9A gRNA targeting sequences and corresponding DNA target sequences

| SEQ ID NO: | gRNA Targeting Sequence (Spacer, RNA) | Target Sequence (DNA) |
|---|---|---|
| 1 | GCUUCUUCUCCUUCUCCAGGCA | GCTTCTTCTCCTTCTCCAGGCA |
| 2 | GAACUUAGCAGUGAUUCGGAUA | GAACTTAGCAGTGATTCGGATA |
| 3 | GAAAACAAGGAGCCACGAAUG | GAAAACAAGGAGCCACGAATG |
| 4 | GAAGAGAUAUAGGAUCUGAGA | GAAGAGATATAGGATCTGAGA |
| 5 | GCCUUGCAGAAAACAAGGAGCC | GCCTTGCAGAAAACAAGGAGCC |
| 6 | GUAAAGGUUUUCCCAGUAAUC | GTAAAGGTTTTCCCAGTAATC |
| 7 | GAAAAAGACCAUUAAGAUUAUCC | GAAAAAGACCATTAAGATTATCC |
| 8 | GAUUAAACCGGUCAAGCUCCU | GATTAAACCGGTCAAGCTCCT |
| 9 | GAGCUCUUUCUAGCAGAUGUGG | GAGCTCTTTCTAGCAGATGTGG |
| 10 | GGAACACCACCCAAUGACUG | GGAACACCACCCAATGACTG |
| 11 | GAAAGCAUAAUGAAUACCCUA | GAAAGCATAATGAATACCCTA |
| 12 | GCUAUCCAUUUUAGAAGCAUUUC | GCTATCCATTTTAGAAGCATTTC |
| 13 | GAUUGUGUUCCGCGUGCUGUG | GATTGTGTTCCGCGTGCTGTG |
| 14 | GAUUGUGUUCCGCGUGCUGUGUG | GATTGTGTTCCGCGTGCTGTGTG |
| 15 | GUGGAGAGUGGAUAGAGACCAU | GTGGAGAGTGGATAGAGACCAT |
| 16 | GUUCCGCGUGCUGUGUGGAGA | GTTCCGCGTGCTGTGTGGAGA |
| 17 | GCGUGCUGUGUGGAGAGUGGA | GCGTGCTGTGTGGAGAGTGGA |
| 18 | GCACGCGGAACACAAUCAGGA | GCACGCGGAACACAATCAGGA |
| 19 | GCGGAACACAAUCAGGAAGGA | GCGGAACACAATCAGGAAGGA |
| 20 | GAAGAAACAGAAGAAAGAAA | GAAGAAACAGAAGAAAGAAA |
| 21 | GUCAAAGCUCGUGUAGCCAUAAU | GTCAAAGCTCGTGTAGCCATAAT |
| 22 | GAACACAGUCAGGAUCAUGACAU | GAACACAGTCAGGATCATGACAT |
| 23 | GAACUUUCAGAGUAUUGAGAGCU | GAACTTTCAGAGTATTGAGAGCT |
| 24 | GCUCUUCGAACUUUCAGAGUA | GCTCTTCGAACTTTCAGAGTA |
| 25 | GUUUCAGCUCUUCGAACUUU | GTTTCAGCTCTTCGAACTTT |
| 26 | GUUUUCAAAGCUCUCAAUACU | GTTTTCAAAGCTCTCAATACT |
| 27 | GAAUAGUGCACAUGAUGAGCAUG | GAATAGTGCACATGATGAGCATG |
| 28 | GCUUUUGAAGAUAUUUAUAUUGA | GCTTTTGAAGATATTTATATTGA |
| 29 | GAUCUUCACUUACAUCUUCAUU | GATCTTCACTTACATCTTCATT |
| 30 | GAUCUUCACUUACAUCUUCAUUC | GATCTTCACTTACATCTTCATTC |
| 31 | GGAAAAAUCUGGUGGAACAU | GGAAAAATCTGGTGGAACAT |
| 32 | GUGAAACAGGCCUCUGGCUCA | GTGAAACAGGCCTCTGGCTCA |
| 33 | GUUAACAUAGAGUCAGGGA | GTTAACATAGAGTCAGGGA |
| 34 | GGAAAAAUCUGGUGGAACAUC | GGAAAAATCTGGTGGAACATC |
| 35 | GCUUCUUCUCCUUCUCCAGGCA | GCTTCTTCTCCTTCTCCAGGCA |
| 36 | GAAGAAGCAGAGGCUGAACCU | GAAGAAGCAGAGGCTGAACCT |
| 37 | GAUUGUUGAACACAGUUGGU | GATTGTTGAACACAGTTGGT |
| 38 | GUUCUCAUGCUGCCAAGUUAACA | GTTCTCATGCTGCCAAGTTAACA |
| 39 | GAAAAAGAUAAAAUCAGUGGUU | GAAAAAGATAAAATCAGTGGTT |
| 40 | GGCACUGUCACUGUGAGGC | GGCACTGTCACTGTGAGGC |
| 41 | GGCUUUUUGGAAAAUGCUUU | GGCTTTTTGGAAAATGCTTT |
| 42 | GAUUUGGAAAAUAUGAAUGCU | GATTTGGAAAATATGAATGCT |
| 43 | GAAUUAAAAAGGGAAUAAAUUAU | GAATTAAAAAGGGAATAAATTAT |
| 44 | GAGAUAAGACAAGCAGAAGAU | GAGATAAGACAAGCAGAAGAT |
| 45 | GCAGUGACUAGAAUUAAAAA | GCAGTGACTAGAATTAAAAA |
| 46 | GAUUGCAGUGACUAGAAUUAAAA | GATTGCAGTGACTAGAATTAAAA |
| 47 | GAUCUUCUGCUUGUCUUAUCUCC | GATCTTCTGCTTGTCTTATCTCC |
| 48 | GAACUUAGCAGUGAUUCGGAUA | GAACTTAGCAGTGATTCGGATA |
| 49 | GUGAGGUUACCUAGAGCCCCUA | GTGAGGTTACCTAGAGCCCCTA |
| 50 | GAUUUUAUCUUUUUCCUUG | GATTTTATCTTTTTCCTTG |
| 51 | GAAGUCGUUCAUGUGCCACC | GAAGTCGTTCATGTGCCACC |
| 52 | GCUCUUUGGUAAGAGCUACA | GCTCTTTGGTAAGAGCTACA |
| 53 | GUUUACAUGAUGGUCAUGGUCA | GTTTACATGATGGTCATGGTCA |
| 54 | GUUUACAUGAUGGUCAUGGUCAU | GTTTACATGATGGTCATGGTCAT |
| 55 | GGUAUUAAAACUGAUUGCC | GGTATTAAAACTGATTGCC |
| 56 | GCCUACUUGGAAAUACUCAU | GCCTACTTGGAAATACTCAT |
| 57 | GUCAAAAAUAUUCCAGCCUAC | GTCAAAAATATTCCAGCCTAC |
| 58 | GUCUUUACUGGAAUCUUUGCAG | GTCTTTACTGGAATCTTTGCAG |
| 59 | GUGGAGCUCUUUCUAGCAGAU | GTGGAGCTCTTTCTAGCAGAT |
| 60 | GACAAUCCUUCCACAUCUGC | GACAATCCTTCCACATCTGC |
| 61 | GUCAAAAAUAUUCCAGCCUACU | GTCAAAAATATTCCAGCCTACT |
| 62 | GAUUUGCACACAAAUUCUUGAUC | GATTTGCACACAAATTCTTGATC |
| 63 | GAAUUCAAAAAUGUACUUGCUA | GAATTCAAAAATGTACTTGCTA |
| 64 | GAAUUCAAAAAUGUACUUGCUAU | GAATTCAAAAATGTACTTGCTAT |
| 65 | GGGAUCAUUCAGCAUAUCCU | GGGATCATTCAGCATATCCT |
| 66 | GCUGAAUGAUCCCAACCUCAGA | GCTGAATGATCCCAACCTCAGA |
| 67 | GAGCACAGCAUUUUUGGAGACA | GAGCACAGCATTTTTGGAGACA |
| 68 | GUUCUGCUGCUUCGCCUUG | GTTCTGCTGCTTCGCCTTG |
| 69 | GAAAACAAGGAGCCACGAAUG | GAAAACAAGGAGCCACGAATG |
| 70 | GAAGAGAUAUAGGAUCUGAGA | GAAGAGATATAGGATCTGAGA |
| 71 | GCAGAGGAAGAGAUAUAGGAU | GCAGAGGAAGAGATATAGGAT |
| 72 | GCCUUGCAGAAAACAAGGAGCC | GCCTTGCAGAAAACAAGGAGCC |
| 73 | GCAGAAUUAUGGGCCUCUCA | GCAGAATTATGGGCCTCTCA |
| 74 | GGAUGUUUCAGAAGAACUCU | GGATGTTTCAGAAGAACTCT |
| 75 | GAAGCUCUCCAGUGGAGAGGAA | GAAGCTCTCCAGTGGAGAGGAA |
| 76 | GGAGAGGAAAAGGGAGAUGCU | GGAGAGGAAAAGGGAGATGCT |
| 77 | GUGCUAAAGAAAGAAGAAACAG | GTGCTAAAGAAAGAAGAAACAG |
| 78 | GCAUCUCCCUUUUCCUCUCCAC | GCATCTCCCTTTTCCTCTCCAC |
| 79 | GAAUCAGAGGACAGCAUCAG | GAATCAGAGGACAGCATCAG |
| 80 | GAUGCUGAGAAAUUGUCGAAAU | GATGCTGAGAAATTGTCGAAAT |
| 81 | GAAUAUACAAGUAUUAGGAGAAG | GAATATACAAGTATTAGGAGAAG |
| 82 | GUGGAGAGGAAAAGGGAGAUG | GTGGAGAGGAAAAGGGAGATG |
| 83 | GUUUAUUAGAUAAAAGGAGCCC | GTTTATTAGATAAAAGGAGCCC |
| 84 | GCAAACAUUGAAGAAGCUAAA | GCAAACATTGAAGAAGCTAAA |
| 85 | GUUUAUUAGAUAAAAGGAGCC | GTTTATTAGATAAAAGGAGCC |
| 86 | GUAAAGGUUUUCCCAGUAAUC | GTAAAGGTTTTCCCAGTAATC |
| 87 | GGCUAAUGACCCAAGAUUACU | GGCTAATGACCCAAGATTACT |
| 88 | GAAUCUGUGCUGAAACCACAA | GAATCTGTGCTGAAACCACAA |
| 89 | GCAUAAAUGUUUUCGAAAUUCAC | GCATAAATGTTTTCGAAATTCAC |
| 90 | GAAACCUGAAGCAUAAAUGUUU | GAAACCTGAAGCATAAATGTTT |
| 91 | GCCAGUUCCACGGGUCACG | GCCAGTTCCACGGGTCACG |
| 92 | GACAAAAUCCAGCCAGUUCC | GACAAAATCCAGCCAGTTCC |
| 93 | GACAAACUGCAUAUUUAUGACC | GACAAACTGCATATTTATGACC |
| 94 | GGUCAUAAAUAUGCAGUUUGU | GGTCATAAATATGCAGTTTGT |
| 95 | GACUUUCAUAGUAUUGAACAAA | GACTTTCATAGTATTGAACAAA |
| 96 | GAAAAUCAAAGGAACCCAAAG | GAAAATCAAAGGAACCCAAAG |
| 97 | GCCCCAAAGCCAAGCAGUGAC | GCCCCAAAGCCAAGCAGTGAC |
| 98 | GGGCUCUGACACCAUGCCGGG | GGGCTCTGACACCATGCCGGG |
| 99 | GAAGAUCUUGUCUGCAUACUC | GAAGATCTTGTCTGCATACTC |
| 100 | GAAAAAGACCAUUAAGAUUAUCC | GAAAAAGACCATTAAGATTATCC |
| 101 | GCCAAGUUAACAUAGAGUCA | GCCAAGTTAACATAGAGTCA |
| 102 | GCUGCCAAGUUAACAUAGAGU | GCTGCCAAGTTAACATAGAGT |
| 103 | GUUAACAUAGAGUCAGGGAA | GTTAACATAGAGTCAGGGAA |
| 104 | GUCAGGGAAAGGAAAAAUCUG | GTCAGGGAAAGGAAAAATCTG |
| 105 | GACUCUAUGUUAACUUGGCAGC | GACTCTATGTTAACTTGGCAGC |
| 106 | GAUUAAACCGGUCAAGCUCCU | GATTAAACCGGTCAAGCTCCT |
| 107 | GAUAACCCUUUGCCUGGAGA | GATAACCCTTTGCCTGGAGA |
| 108 | GUGAAACAGGCCUCUGGCUCAU | GTGAAACAGGCCTCTGGCTCAT |
| 109 | GCAAAGGUCACAAUUUCCUCA | GCAAAGGTCACAATTTCCTCA |
| 110 | GCACAGUUGAUAACCCUUUGCC | GCACAGTTGATAACCCTTTGCC |
| 111 | GGGGAAUCCGAUUUGGAAAAU | GGGGAATCCGATTTGGAAAAT |
| 112 | GCAGAAGAUCUGAAUACUAAGA | GCAGAAGATCTGAATACTAAGA |
| 113 | GUCACUGUGAGGCUGGGAUU | GTCACTGTGAGGCTGGGATT |
| 114 | GCAGAAGAUCUGAAUACUAAG | GCAGAAGATCTGAATACTAAG |
| 115 | GCAGCGUGGACAAACACUUG | GCAGCGTGGACAAACACTTG |
| 116 | GAGCAAAGGUCACAAUUUCCUC | GAGCAAAGGTCACAATTTCCTC |
| 117 | GUGACAGUGCCAAUUGCACCUG | GTGACAGTGCCAATTGCACCTG |
| 118 | GCUGAGGAACUUAGCAGUGAU | GCTGAGGAACTTAGCAGTGAT |
| 119 | GCAAUCUGGAGGUUGUUUGCAU | GCAATCTGGAGGTTGTTTGCAT |
| 120 | GUGACAGUGCCAAUUGCACCU | GTGACAGTGCCAATTGCACCT |
| 121 | GAUUGCAGUGACUAGAAUUAAA | GATTGCAGTGACTAGAATTAAA |
| 122 | GCACCUGGGGAAUCCGAUU | GCACCTGGGGAATCCGATT |
| 123 | GUGACAGUGCCAAUUGCACC | GTGACAGTGCCAATTGCACC |
| 124 | GCUCAUUUCAGCAAGUGUAUGGU | GCTCATTTCAGCAAGTGTATGGT |
| 125 | GAGCUCUUUCUAGCAGAUGUGG | GAGCTCTTTCTAGCAGATGTGG |
| 126 | GGUAAUUGCAAGAUCUACAA | GGTAATTGCAAGATCTACAA |
| 127 | GUGUAUCUAUUUUAUUGUA | GTGTATCTATTTTATTGTA |
| 128 | GAUCUGGAAUUGCUCUCCAUA | GATCTGGAATTGCTCTCCATA |
| 129 | GGAACACCACCCAAUGACUG | GGAACACCACCCAATGACTG |
| 130 | GAUUUGCACACAAAUUCUUGAU | GATTTGCACACAAATTCTTGAT |
| 131 | GGAACACCACCCAAUGACU | GGAACACCACCCAATGACT |
| 132 | GCAUAUCCUCUGAAAGGAGA | GCATATCCTCTGAAAGGAGA |
| 133 | GUAGUUCCUAUCUCCUUUCA | GTAGTTCCTATCTCCTTTCA |
| 134 | GAUCAUUCAGCAUAUCCUCUGAA | GATCATTCAGCATATCCTCTGAA |
| 135 | GCAAGCAUAUUAACAAACACU | GCAAGCATATTAACAAACACT |
| 136 | GUCCCCACCAAUGCUGCCGGUGA | GTCCCCACCAATGCTGCCGGTGA |
| 137 | GGGCUGAGCGUCCAUCAACCA | GGGCTGAGCGTCCATCAACCA |
| 138 | GAGGGCUGAGCGUCCAUCAAC | GAGGGCTGAGCGTCCATCAAC |
| 139 | GAUGAUGAGCACAGCAUUUU | GATGATGAGCACAGCATTTT |
| 140 | GUCUUUUUAGUUUCAAAGGCA | GTCTTTTTAGTTTCAAAGGCA |
| 141 | GCUCUCCAGUGGAGAGGAAAA | GCTCTCCAGTGGAGAGGAAAA |
| 142 | GAAGCAGAAUUAUGGGCCUCU | GAAGCAGAATTATGGGCCTCT |
| 143 | GGGCCUCUCAGAGAGUUCUU | GGGCCTCTCAGAGAGTTCTT |
| 144 | GAAUCAAAAGAAGCUCUCCAG | GAATCAAAAGAAGCTCTCCAG |
| 145 | GGGAGAUGCUGAGAAAUUG | GGGAGATGCTGAGAAATTG |
| 146 | GCCUAUGCCCUUCGACACCAAGG | GCCTATGCCCTTCGACACCAAGG |
| 147 | GCCUAUGCCCUUCGACACCAAG | GCCTATGCCCTTCGACACCAAG |
| 148 | GCUCUAAAAGUGCUAAAGAAAG | GCTCTAAAAGTGCTAAAGAAAG |
| 149 | GAAGAAGCUAAACAGAAAGAAU | GAAGAAGCTAAACAGAAAGAAT |
| 150 | GUACACCUGUGUGAAAAUUGG | GTACACCTGTGTGAAAATTGG |
| 151 | GGCUAAUGACCCAAGAUUA | GGCTAATGACCCAAGATTA |
| 152 | GGCUAAUGACCCAAGAUUAC | GGCTAATGACCCAAGATTAC |
| 153 | GUGUCCAGAGGGGUACACCUGU | GTGTCCAGAGGGGTACACCTGT |
| 154 | GAAACCACAAAGGAGAGCAUCU | GAAACCACAAAGGAGAGCATCT |
| 155 | GAAAGCAUAAUGAAUACCCUA | GAAAGCATAATGAATACCCTA |
| 156 | GUUUCAUUAUUUUCAAGUGAAUU | GTTTCATTATTTTCAAGTGAATT |
| 157 | GAAAGCAUAAUGAAUACCC | GAAAGCATAATGAATACCC |
| 158 | GCAAGAGGCUUCUGUGUAG | GCAAGAGGCTTCTGTGTAG |
| 159 | GUUCCACGGGUCACGAAGAAAA | GTTCCACGGGTCACGAAGAAAA |
| 160 | GAAUUCACUUUUCUUCGUGACCC | GAATTCACTTTTCTTCGTGACCC |
| 161 | GAUAUUCUUCUUAGAGGACUGAA | GATATTCTTCTTAGAGGACTGAA |
| 162 | GCAUAUAUAAAGCAGGUGUGGCA | GCATATATAAAGCAGGTGTGGCA |
| 163 | GUUUUCCCUUUGUUCAAUACU | GTTTTCCCTTTGTTCAATACT |
| 164 | GCAUUGCUGAAAGAAAAUCA | GCATTGCTGAAAGAAAATCA |
| 165 | GCAAACAGCUGCCCUUCAUCUA | GCAAACAGCTGCCCTTCATCTA |
| 166 | GGACAAAGCUCUGAGGUCCU | GGACAAAGCTCTGAGGTCCT |
| 167 | GAGGGCAAGAGACUGUUUU | GAGGGCAAGAGACTGTTTT |
| 168 | GCCCUCAUUGAACAACGCAUUG | GCCCTCATTGAACAACGCATTG |
| 169 | GCAAUGCGUUGUUCAAUGAGGGC | GCAATGCGTTGTTCAATGAGGGC |
| 170 | GGGGCUCUGACACCAUGCCGG | GGGGCTCTGACACCATGCCGG |
| 171 | GCUGCCAAGUUAACAUAGAGUC | GCTGCCAAGTTAACATAGAGTC |
| 172 | GGCACGACCAAUCAAAUACAC | GGCACGACCAATCAAATACAC |
| 173 | GAUGUUACUGCUGCGUCGCUCC | GATGTTACTGCTGCGTCGCTCC |
| 174 | GCGGCUGAAUAUACAAGUAUU | GCGGCTGAATATACAAGTATT |
| 175 | GUCGAAGGGCAUAGGCGAGCAC | GTCGAAGGGCATAGGCGAGCAC |
| 176 | GACAUUUUUGGUCCAGUCCG | GACATTTTTGGTCCAGTCCG |

2. SCN10A gRNA targeting sequences and corresponding DNA target sequences

| SEQ ID NO: | gRNA Targeting sequence (spacer, RNA) | target sequence (DNA) |
|---|---|---|
| 177 | GAGAAGGCAUAGAAUAAAGCC | GAGAAGGCATAGAATAAAGCC |
| 178 | GGUGUUCAUCUUCUCCAUGCC | GGTGTTCATCTTCTCCATGCC |
| 179 | GUGAGAGGAAAGCCCAAGCAA | GTGAGAGGAAAGCCCAAGCAA |
| 180 | GUCACAUCAGCCAGUUUCUUCA | GTCACATCAGCCAGTTTCTTCA |
| 181 | GAACUGAUCGGGGAGCCCCUG | GAACTGATCGGGGAGCCCCTG |
| 182 | GGGAUCCGUCACAAGCCCAAA | GGGATCCGTCACAAGCCCAAA |
| 183 | GUCCGCAGGCCUGAGAUCCCA | GTCCGCAGGCCTGAGATCCCA |
| 184 | GGGCACUUCUGUUCAGACUCC | GGGCACTTCTGTTCAGACTCC |
| 185 | GGAUACGGAAGACAAUGAGGA | GGATACGGAAGACAATGAGGA |
| 186 | GUUCUCAAUCCACUCUCCACA | GTTCTCAATCCACTCTCCACA |
| 187 | GUUAGUUUCGAGGGAUCCAAUG | GTTAGTTTCGAGGGATCCAATG |
| 188 | GCCGAGAUAUCUCACUCCCUGA | GCCGAGATATCTCACTCCCTGA |
| 189 | GCCUUGAUAAAGAUACUGGCAA | GCCTTGATAAAGATACTGGCAA |
| 190 | GUCAAGAAAAGGAUGAGGCAU | GTCAAGAAAAGGATGAGGCAT |
| 191 | GUGUCAUGAGGCGGAACAGUGA | GTGTCATGAGGCGGAACAGTGA |
| 192 | GGGGAAAACUACCGUAACAA | GGGGAAAACTACCGTAACAA |
| 193 | GAGAAGGCAUAGAAUAAAGCC | GAGAAGGCATAGAATAAAGCC |
| 194 | GAGGUUAAAGGUGAUCCAUUGU | GAGGTTAAAGGTGATCCATTGT |
| 195 | GGUGAUGGUGAGCUCUGCAA | GGTGATGGTGAGCTCTGCAA |
| 196 | GGUGUUCAUCUUCUCCAUGCC | GGTGTTCATCTTCTCCATGCC |
| 197 | GGAGGUUAAAGGUGAUCCAUU | GGAGGTTAAAGGTGATCCATT |
| 198 | GGGGCUCCCCGAUCAGUUCUG | GGGGCTCCCCGATCAGTTCTG |
| 199 | GGAAGACAAUGAGGAAAGA | GGAAGACAATGAGGAAAGA |
| 200 | GGAAACUCAGUGGGGGCAC | GGAAACTCAGTGGGGGCAC |
| 201 | GUGAGAGGAAAGCCCAAGCAA | GTGAGAGGAAAGCCCAAGCAA |
| 202 | GUCACAGAUGAUGGAGUCUUUCC | GTCACAGATGATGGAGTCTTTCC |
| 203 | GGAGUCAGGGUUGCUGGGUU | GGAGTCAGGGTTGCTGGGTT |
| 204 | GUCACAUCAGCCAGUUUCUUCA | GTCACATCAGCCAGTTTCTTCA |
| 205 | GAACUGAUCGGGGAGCCCCUG | GAACTGATCGGGGAGCCCCTG |
| 206 | GGCAAGAGGAUUUUGUCUAA | GGCAAGAGGATTTTGTCTAA |
| 207 | GCUCUGAUCCUUACAACCAGCG | GCTCTGATCCTTACAACCAGCG |
| 208 | GUAUCCUCUGUGGAGAGUGGA | GTATCCTCTGTGGAGAGTGGA |
| 209 | GAAGCCUCGGCCCCAGCUGGAC | GAAGCCTCGGCCCCAGCTGGAC |
| 210 | GACAACCAACUACUCAUCUCA | GACAACCAACTACTCATCTCA |
| 211 | GUCUAAAUGAGUUCACGUAC | GTCTAAATGAGTTCACGTAC |
| 212 | GCAUAGGGAGCAGAAGGACCAAG | GCATAGGGAGCAGAAGGACCAAG |
| 213 | GCUAGAGCUGGGCGUGGCCAAGA | GCTAGAGCTGGGCGTGGCCAAGA |
| 214 | GUCAGAUCCAUUGCCACACAGUA | GTCAGATCCATTGCCACACAGTA |
| 215 | GGGUUGAGGAAGAGGGCUUC | GGGTTGAGGAAGAGGGCTTC |
| 216 | GCCUUAAAACUUCUGACAAC | GCCTTAAAACTTCTGACAAC |
| 217 | GAUGGAGUCUUUCCUGGAGACC | GATGGAGTCTTTCCTGGAGACC |
| 218 | GCCUCAUGACACAGGAUUC | GCCTCATGACACAGGATTC |
| 219 | GCAUGACCCGAACUGACCUUC | GCATGACCCGAACTGACCTTC |
| 220 | GCCAAGAUCAAGUUGACCAGG | GCCAAGATCAAGTTGACCAGG |
| 221 | GCUCUGGUUGGCAAGCAGCUCCU | GCTCTGGTTGGCAAGCAGCTCCT |
| 222 | GCAACCCUGACUCCAGGCA | GCAACCCTGACTCCAGGCA |
| 223 | GGGAUCCGUCACAAGCCCAAA | GGGATCCGTCACAAGCCCAAA |
| 224 | GCCAGCACCCCCACCCAGCAG | GCCAGCACCCCCACCCAGCAG |
| 225 | GUUAGUUUCGAGGGAUCCAA | GTTAGTTTCGAGGGATCCAA |
| 226 | GUUUUUAAUGCUCUAAGAACU | GTTTTTAATGCTCTAAGAACT |
| 227 | GGAGCAUCUCGAGGGCCUC | GGAGCATCTCGAGGGCCTC |
| 228 | GGACUGAAUAGCCACAGGGC | GGACTGAATAGCCACAGGGC |
| 229 | GAGAAGCAAAUUGCUGCCAAG | GAGAAGCAAATTGCTGCCAAG |
| 230 | GUUCACGUACCUGAGAGAUCC | GTTCACGTACCTGAGAGATCC |
| 231 | GAGAGAUCCUUGGAACUGG | GAGAGATCCTTGGAACTGG |
| 232 | GCUUUACUCCGGAGUCACUGG | GCTTTACTCCGGAGTCACTGG |
| 233 | GCUAGAGCUGGGCGUGGCCAAG | GCTAGAGCTGGGCGTGGCCAAG |
| 234 | GCGCUGGUUGUAAGGAUCAGAG | GCGCTGGTTGTAAGGATCAGAG |
| 235 | GUUCCCUAGCACCAUCACCGUC | GTTCCCTAGCACCATCACCGTC |
| 236 | GUCCGCAGGCCUGAGAUCCCA | GTCCGCAGGCCTGAGATCCCA |
| 237 | GCCUCAUGACACAGGAUUCC | GCCTCATGACACAGGATTCC |
| 238 | GUUCCAUUUCCGGUCCCCUGG | GTTCCATTTCCGGTCCCCTGG |
| 239 | GCGACGGAAGUUGUUAGUUUC | GCGACGGAAGTTGTTAGTTTC |
| 240 | GGGCACUUCUGUUCAGACUCC | GGGCACTTCTGTTCAGACTCC |
| 241 | GAUGGCUUUCGUGGUCUCC | GATGGCTTTCGTGGTCTCC |
| 242 | GCAACCUCAAAAAUAAAUGUGUC | GCAACCTCAAAAATAAATGTGTC |
| 243 | GCCCCGAUGGCUUUCGUGGUCUC | GCCCCGATGGCTTTCGTGGTCTC |
| 244 | GGCACAGCAAUAGAUCUCCG | GGCACAGCAATAGATCTCCG |
| 245 | GGAUACGGAAGACAAUGAGGA | GGATACGGAAGACAATGAGGA |
| 246 | GAAAGUCUUCUUUUGUCCUGCA | GAAAGTCTTCTTTTGTCCTGCA |
| 247 | GGAAACUCAGUGGGGGCACU | GGAAACTCAGTGGGGGCACT |
| 248 | GUGAGAUAUCUCGGCCAGGGGA | GTGAGATATCTCGGCCAGGGGA |
| 249 | GCCAAGCAGGGAACAAAGAAAGC | GCCAAGCAGGGAACAAAGAAAGC |
| 250 | GAGAAGCAAAUUGCUGCCAAGC | GAGAAGCAAATTGCTGCCAAGC |
| 251 | GCACCAUCACCGUCAAGAA | GCACCATCACCGTCAAGAA |
| 252 | GGGGCUCUCUGCUGCUGGGU | GGGGCTCTCTGCTGCTGGGT |
| 253 | GUCAAAGAUAUUCCACUUCUUC | GTCAAAGATATTCCACTTCTTC |
| 254 | GUCUUCCGUAUCCUCUGUGGAGA | GTCTTCCGTATCCTCTGTGGAGA |
| 255 | GACCCCUUACUGUGUGGCA | GACCCCTTACTGTGTGGCA |
| 256 | GGUUGGCAAGCAGCUCCUAG | GGTTGGCAAGCAGCTCCTAG |
| 257 | GAUCUCAGGCCUGCGGACAUU | GATCTCAGGCCTGCGGACATT |
| 258 | GGGAUCCGUCACAAGCCCA | GGGATCCGTCACAAGCCCA |
| 259 | GCGUUUUCCAGAGGCGAGGCC | GCGTTTTCCAGAGGCGAGGCC |
| 260 | GAGAUAUCUCGGCCAGGGGAC | GAGATATCTCGGCCAGGGGAC |
| 261 | GCCAUCGGGGCUCUCUGCUG | GCCATCGGGGCTCTCTGCTG |
| 262 | GUUAGUUUCGAGGGAUCCAAU | GTTAGTTTCGAGGGATCCAAT |
| 263 | GAUUGAGAACAUGUGGGCCUGC | GATTGAGAACATGTGGGCCTGC |
| 264 | GGAAUUCCCCAUUGGAUCCC | GGAATTCCCCATTGGATCCC |
| 265 | GUUCUCAAUCCACUCUCCACA | GTTCTCAATCCACTCTCCACA |
| 266 | GUCAAAGAUAUUCCACUUCUU | GTCAAAGATATTCCACTTCTT |
| 267 | GUCUUCCGUAUCCUCUGUG | GTCTTCCGTATCCTCTGTG |
| 268 | GCAGGGAACAAAGAAAGCCA | GCAGGGAACAAAGAAAGCCA |
| 269 | GAACUCUGAAUGUCCGCAGGC | GAACTCTGAATGTCCGCAGGC |
| 270 | GACUCCAUCAUCUGUGACUCCCU | GACTCCATCATCTGTGACTCCCT |
| 271 | GACAAUUCUCUUUGGGCUUGUG | GACAATTCTCTTTGGGCTTGTG |
| 272 | GGUGAUCCAUUGUGGGAGUG | GGTGATCCATTGTGGGAGTG |
| 273 | GGUGAUGGUGAGCUCUGCA | GGTGATGGTGAGCTCTGCA |
| 274 | GUGACUCCGGAGUAAAGCG | GTGACTCCGGAGTAAAGCG |
| 275 | GUUAGUUUCGAGGGAUCCAAUG | GTTAGTTTCGAGGGATCCAATG |
| 276 | GGUUGGCAAGCAGCUCCUA | GGTTGGCAAGCAGCTCCTA |
| 277 | GAGAAGAAGUUCCAGGAGGCCC | GAGAAGAAGTTCCAGGAGGCCC |
| 278 | GCAAUCCCAGAUCAGAUACUU | GCAATCCCAGATCAGATACTT |
| 279 | GCGACGGAAGUUGUUAGUUUCG | GCGACGGAAGTTGTTAGTTTCG |
| 280 | GGGCCCGAGUGGCACUAAAC | GGGCCCGAGTGGCACTAAAC |
| 281 | GAGCAGAACCAGGCAACCACUG | GAGCAGAACCAGGCAACCACTG |
| 282 | GGCCGAGAUAUCUCACUCCCU | GGCCGAGATATCTCACTCCCT |
| 283 | GCCGAGAUAUCUCACUCCCUGA | GCCGAGATATCTCACTCCCTGA |
| 284 | GUGAGGUUCCCCAGUGCCCCCA | GTGAGGTTCCCCAGTGCCCCCA |
| 285 | GCCUUGAUAAAGAUACUGGCAA | GCCTTGATAAAGATACTGGCAA |
| 286 | GGGGCCAGUCUUCAUGGGGCG | GGGGCCAGTCTTCATGGGGCG |
| 287 | GCUAAAAUCCAGCCAGUUCC | GCTAAAATCCAGCCAGTTCC |
| 288 | GGGGCCCUGAUUCACUCAGUG | GGGGCCCTGATTCACTCAGTG |
| 289 | GAUCUGGGAUUGCUGCCCCAU | GATCTGGGATTGCTGCCCCAT |
| 290 | GUCUUUACCAUAUUUUUUACUG | GTCTTTACCATATTTTTTACTG |
| 291 | GUUCAUCUAAGUAUUCUGCUGAC | GTTCATCTAAGTATTCTGCTGAC |
| 292 | GUCUAAAUGAGUUCACGUACCU | GTCTAAATGAGTTCACGTACCT |
| 293 | GUGAGCUCCCAGCAGAACUGAU | GTGAGCTCCCAGCAGAACTGAT |
| 294 | GUCAAGAAAAGGAUGAGGCAU | GTCAAGAAAAGGATGAGGCAT |
| 295 | GAGGAUACGGAAGACAAUG | GAGGATACGGAAGACAATG |
| 296 | GGGAGUGAGAUAUCUCGGCC | GGGAGTGAGATATCTCGGCC |
| 297 | GCUUGUCUCAGAAGUAUCUGAUC | GCTTGTCTCAGAAGTATCTGATC |
| 298 | GGGCCCGAGUGGCACUAAACC | GGGCCCGAGTGGCACTAAACC |
| 299 | GUUGACCAGGUAGAAAGAUCC | GTTGACCAGGTAGAAAGATCC |
| 300 | GCUUGUCUCAGAAGUAUCUGAU | GCTTGTCTCAGAAGTATCTGAT |
| 301 | GAUGAGUGUGUUUAAGGUGGGC | GATGAGTGTGTTTAAGGTGGGC |
| 302 | GUGAGAGGAAAGCCCAAGCA | GTGAGAGGAAAGCCCAAGCA |
| 303 | GCAUGACCCGAACUGACCUUCCA | GCATGACCCGAACTGACCTTCCA |
| 304 | GUUGGCACAGCAAUAGAUCUCC | GTTGGCACAGCAATAGATCTCC |
| 305 | GUCAAGAAUGACAUGGCUGUCA | GTCAAGAATGACATGGCTGTCA |
| 306 | GUGGGCCAGGAUUUGGCCAGC | GTGGGCCAGGATTTGGCCAGC |
| 307 | GCAAAGACAAAGACAAUGAUGGC | GCAAAGACAAAGACAATGATGGC |
| 308 | GUCAUGAGGCGGAACAGUGAG | GTCATGAGGCGGAACAGTGAG |
| 309 | GAUGGCCGUUCUUCUGAUCAGG | GATGGCCGTTCTTCTGATCAGG |
| 310 | GGGCCCGAGUGGCACUAAA | GGGCCCGAGTGGCACTAAA |

>NG_012798.1: 5001-185803 Homo sapiens sodium voltage-gated channel alpha
subunit 9 (SCN9A), RefSeqGene (LRG 369) on chromosome 2
SEQ ID NO: 311
CGGGGCTGCTACCTCCACGGGCGCGCCCTGGCAGGAGGGGCGCAGTCTGCTTGCAGGCGGTCGCCAGCGC
TCCAGCGGCGGCTGTCGGCTTTCCAATTCCGCCAGCTCGGCTGAGGCTGGGCTAGCCTGGGTGCCAGTGG
CTGCTAGCGGCAGGCGTCCCCTGAGCAACAGGAGCCCAGAGAAAAAGAAGCAGCCCTGAGAGAGCGCCGG
GGAAGGAGAGGCCCGCGCCCTCTCCTGGAGCCAGATTCTGCAGGTGCACTGGGTGGGGATGATCGGCGGG
CTAGGTTGCAAGTAAGTGCCTTTTCTTTTGCTGCTTCTGTGGGGAGGGGAGGAGAAGCCCTCGGTCTTTC
GCCCGTGCTCGCCTCAACCGCCACACCCACCTGTGTCTATTTTCACATCTCGGTTCTGGAACCCTGTATT
ACGCCACCTGGAAAGAAGAGAGGGGAGAAGCTTGACCGGGTGGTTCCAGCAATGGGAGGAAGAAGGGCTG
GAAGCGTAGTTATTTCGATAGCTGGGCAGCTCCTGTGCGGTTTTGACTCTGTGGGTTTGCTGCCAGGTTC
TGAAACTGTGGAAAGGAAGGGTAGCAATGCCTGGTGGTTTACTGTCTTTTATTTCTGTTTACGCTAGCAG
AGTTGTTAGATGGACTTCAATTCAAGGGGCAGCTCACATTTTTTACGAGCAAAAAGCATGTGCTGTATTT
CCCATCTGCAAAGCTGAGCGTGCAGAACCAGCTTAGCAGATACAACCTGTGGGGACAGAAAAGAACCCCG
CACCCCTTAAGAGCACACAAACACAAATAATTGACAGGACAGCTCTGCCCCTATTTCTCAGCGCAGATTA
TTTATTTATTTTTTCGAATAAGCACTTTATAGTGAAATTCAGAAAAGATGCAGAATCCTTTTAACTTTTT
ATCGAATTCAACTTGGGAAACTCATTTACTTTTAGGAAAAAATGGATATTTTCGTGCCTTATATTCAAGA
CAAAAATCAGAACAAGAAACTAGAAACATTAATGCCAGTTTTTGTGATTATAGTAAACATTTCACTCATT
ATTTTAGTTTTTTTTTTTTAAGTTGGAGCGTTCATAGATTATATCTACTCTGATGTTAAGAGCAATTTAC
ATGAGAAACTTAGGGGGGTTTTTAAGAAGCTAGTGTCAGTAAGATAAAAGCCAAATCAAAGTAAGCTTTC
AAGGAAGTGATAGCTTGTCAGTTACTGAATAGGAAGTCAGTGTTGGGAAGTTCAGTTCTTTTCAGAAACA
TATTTATTATTCTTTTCTATTTCTTCTTCCCTGTAGACAAGATAAAGATTTTCTTTCTCTTTCTTTCCTT
GTTATTTAAATATATTTTATAATTTTTGCTTTAACATCAATATAAACCTATTTTATATAAACTGCAAACC
AAATCCAATTATTAAACTGTTCATTTTATTGCAAATTCTTCAGATTTTTTCCACTATTTAAAAAAGTTTA
TAATTTTTTCTTATAATTATGGTGAGATTTTTTAAAAATTATTGATTAGTTTGAAAGCTAGTCTTATCTG
ATCCCTGTGTCGCAAATTTTCATGATTGTTAGACTAGCTTATCAACAACAAAACTCTAGACAGAATATGG
TATTTGATGTAAAGAAGAATGTGCCCTCTATTTAGGCAACTGATAGTTTGGCTTAAGACAAGTTTTTTAA
AATTTTGTAGAGGTTTAAAAGGAGGTTTCCTTAAGTAAGCCACTGTGCAGTTGTGGGACTCAGTGATGTG
TTTTTACAGTGACTGGATATATGATATGCCTTGATTCCTATGGTGCAGGGCTAGGCTCACAGAGTACCTG
ATGCTTCTACAGAAGAAAGATGAGTCAGGTCTGGCTCTCTGCCAAAAGCATCTTGGGCCTCCTTTCCCCT
AGGGCCTGCATATAATCATGGGAGCCCTTCTGTGGTAATGCTTCTATGTTTTTACTACCTGTTGCTGTGC
TGAGTTTCAGAAGCAAAATAGATAAACAAATAAATAAATTCAGAAGAGCAATCAGTAGGGTTCTATTTAT
TTAATATAATCAACCCACTTTCTCTGTGGCAAGGAAAAAAGTAGAATTTTTCATTTTACATGAATAATAA
AATCTATTTTTTATCCAATTTCTATTCCTATTAGTTATGCAAAAGAGTGTGAAGTGCTAGAAGGATTTAT
ATGGTACTTTTCTAAGACAAGTCATGATCTTGTAAAAGAGCTTAAAGTTTATTGATGTAAATGGAACATT
CTAGATGTCATAATGATAATAATTTATGACTTTCCCTAAAACTCTGTCTCAGATTCTTTCCTCCCATCTT
CATTATCTGAATAAATGCCTTCACCATCCAAGTATTGAAATCACCTTTGATTGCTCTCTTCCTTATTACT
CCATTTCAGCCCTTTCCAGCCTGTAAGTAGATCTATCAATCTTATCTCCCAAACTTATTTCTATTTCATC
ATACGTCTCAGATGCCTCTCATTCACCCTCTTTTCTGTATCTACACGGCCACCAAATTAGTTCAGGTCAT
CATCATAATTTACTTGGAATCCTGCCACAGCCTCCATATCAATCTCTCCACTTCTGTAATTTTCTCCCCT
GCAATTCATTCTCTATACAAAGCAATACCATCCTGTCTCCCCTAACAAACATTGGCTCACAACTGCATTT
AAAAAAAAAAAAAAAGAGGAAGAAGAAGAGGAGAAGAAGAAATAAACATGGCAACACAGAGACCAATTAG
CAACAACAGTAAATGCCTTTCCTCAGCATATGGTTTACTCTCTGTTCTGTCCCTTCTCTCCAACCTCACC
AGAACCTATTCTCTTCTCATTAAGGTGCTCCTGCCCAGGTCGTGGCTAAAACACATGAAACAATAGCACT
CTTTAAGGCCTTTTCAAATGCTGTTCCCAATGCCTGAACTCTATGCGTGCTTACTCCTTCTTAAACTTTA
AATCTCAACTCGAGGTTTTTTTGTTTTGGAAAGATCATTTTTGATCATCTTGTTAAAAGTAGGTTGTCCC
GTGTTGGTCTTGATGTTGGAACCTTGTTTCTTTCATACTGCTTGTTACAGCATGCAGGTATTTTGTGAAT
TTGCATACCAAATACTCCAAGGGAACCCAGACTATGTCTTCATTGTTGACTTAGCTTCCAGAATAGTGCC
TGGCATCAAGTAGTAGGGGGTTCGTAGATATTTATATAAATAATAAAATACAAAAATATGGATTCTTCCA
GAATAAAGCATATTAACCCTGGAAACATGAATTTTGTGATTCTATTTTTTATTTGTCAAGCATTTAGTAT
TTTTACAATATCATGGTATGATATTGAGGAGGTAAAATTAAAATATCCTGAGATGTATTCTCTCTTTTAT
CAGTGTAGGTGATAATGTACTTAAAGGTAAGTAATTTGTAATGCATTCTTAGAAAGGCAGTGACCACAAA
TGTATTAAAATATATTTTAAGAAAAAATTGGATTGCATAAACTAATATGGTTATAAGTGACTCCAAATCT
AAAATGATAACCTTGTTAGGTTTAAATCTGTATCATCTAACAGAAATTTGAGGGCCAGTCAATAGTCACT
GACTATGAATTTAAAATATTCTTTTAAAATAAACATAGATGCTTATGTGAGCTCAGTTGATAGAAATGTA
GTCAGATGAGGCGGAGGCCATAAGTTAGGTTTCCACACGGTCTAAATTGATGTTTGCCCCAGGTACAAAG
GGCACATTATCCAAATATTTGAAGCATTCTCTAAGGAAGGTTCAGAGAGGCAATGTGGGCTTATCCATGA
AGACCCATCACTGTTATTAGAGTGGAAGTAAGGACTGCAAACTTCATGCTCATAAATGATTGTGTCATTT
TCATACTAGAATTTTACCACACACACACTCACACTCACACACACACACACACACACTTTGTATGGTGATT
TGCCTCCCCCCTTTATTTTTGCAGACTATGCTTAAAGATATTAAACAAACAACTAGTTTTGCTATTAACA
TTTGGTTGAAAATTTAGCTTTCAATTAGGTGACAAACTGATTAATGATTTTTTTATGTAGCAAATGGCCA
GCCACAGATTTTTGGTAAATAATTTGGTTACCACATTACATCTTGAAGGACTAAATTATTTCACTAATTG
TGAGCAGGGATTTTTAAAAATTTGCTCTGAAGAGGACATATTGTGGATGTTGTATTGGAGGGCATCTGTG
TACTAATTGAAGATGGTCCTTCATATTACACATCACAAATTAGCTAATTTATTCTTTACAGAAGTATTAT
GACATCTTCCTTTAGAACTGTGTAGAGTTAGATTACCACTTGAGAGGCAAGGTCATATTAAAATAGATAT
TATAAAATTGTATAAAAATATTTTATATGAGGATTTATGAGAGCATCCAGAGAATGTGGGTAGAATACTT
TTGTGGAATATGAGATAACAATTTTGGTGTCCTGCCATTCTAAAAAATGAAATTTCTTCATTGAAGGTAT
CACTTACCCTTTCCTCTGCCTTGCTGGAAGGAAATGGCTTTAGGCAAAAGAAAAAGCCATCAGCTCTTGG
CAGCTAAAGGTGGAATGAAGTTTGGAGGAATGAAACCCTTCTGGGTGGTGAGGAAAAATGCCAGGCCATG
ATAAAGTGGGGAAGGAAGAGGGGAATGCCTTGAACAGTGCCAGTAAGCATAATCTGCCACAGACAAGTTT
GAATTGACATATGTAGAACTGAATTATTCTATATCTTTTTAGTTCTATAGAGAAAACCGTTCTTTCCACG
TCTCTTTTTAAAATAACAACTTACTGTAAGCAAAAAATATGTTGTTGTTGTTTCTTTAACACAGGAGATG
AACTATCTTTAGATTTTTTAAAGTGATATAACATGGAGCCAAAAATGCCATATATTACTGTTGAAACCTA
ACGTTACTTTAACTGTAATTTCCTTGCTATTCATTTTATGATTTCCTCTTGTTATGTCAAGTGTTAGAGA
GACTGCTATTACTGATCATAAATTTCTTTAAAATGTCTCCTTGGATTTTTTACAAAGCTTTCTGAATCAT
TTATCAAAATACAATAAAGCTCTAATTGCACCTAGGATTGTTCACTTGGAAATTTAGAAAGAGTGTGTAC
ATTTACAATTAAGTTAGCATCCTGCCCTGCAATGAATGCCCTTTTTCTTTTTTTTTTTCCTTCTTCTCTC
CTACATCTCATCTTCCAAATGTTTGATATGTACGCCAGTAAGGCAAATTTTACTCCACTTTTAGGTCAAA
CTATTAGTACAAATAATTGGAAAATTGAGCCTCTAAAGAAAGATAAATAAGATTAGAAGCATGCATTAAA
ATATAAGTATTAAAAACATTCCCATAAGATTTTTAATAAAAATTTTATGTGCTATATGCACACCTAGACT
CCCTGTATTATCTGATTTTTAAAGATTCTTTTTTTTTTTTATTTTTTATTTTTTTGAGACGGAGTCTCGC
TCTGTCGCCCAGGCTGGAGTGCAGTGGCGCGATCTTGGCTCACTGCAAGCTCCGCGTCCTGGGTTCACAC
CATTCTCCTGCCTCAGCCTCCGAGTAGCTGGGACTACAGGCGCCTACCACCGTGCCCGGCTAATGTTTTT
GTATTTTTAGTAGAGACGGAGTTTCACCATGTTAGCCAGGATGGTCTCAATCTCCTGACCTAGTGATCCG
CCCGCCTCGACCTCCCAAAGTGCTGGGATTACAAGCATGAGCCACCGCGCTCAGCCCATTATCTAATGAT
GATGATGATGATTATTATTATTATTATTATTATTATTATTATTATTATTATTATTTTAACTGGGGGTAAT
TTCACTGGATGTGGTATCTGTCAACTTTTACTAGTTGCTCCTACACTATATGTTTTTAAATCCAATGTAA
TTATTTTAAAAATATGTCATCTTCTTGAGAGTCTATACTTTAGAATTTGAAAAAAGAAGTAAATGTAGAT
ATTCAAATCCATGCAGATGAGTAAAACAGGGAAAAATGAGAAAAATGTTTCATGATAATAAAATATTATG
TCAGGCACAGCTGGTCACCCCTGCAATCCCATCACTTTGGGAGGCCGAGGCTGGAGGATTGCTTGATCCA
GGAGTTCTAGATCAGCCTGGGCAATAAAAGGAAAATCTGTTTCTACAAAAAAATTAAAATTTATATTTCA
TATATTGATTTAAGCTCTACTAAATGACTATTCTTTCAAGTCTAATAGACAGTGTATCTTTTAGGGCTCA
GAGTAAAGATAATTTATTGAGGACATTTTGAATAGGATGAATTAAATGCTTTATTGGTAAGTGATGCCTT
TATCTGAGTGATTCCTTCTAGGTAAATATTATGGGACCAATCTTTGTAAATGATCTTTGCATGGAACAAA
ATATTATAAAGTTGAAGTGATGAATTTGTTTCCCTCTTAACAAAATATGTATTCTTGTTACCCCATTTAC
AAACTTGGCTCATGGTAATTCAGGTAGGAAAAAGGGAAGTGGCCTACTCAGGGATCAACTGGGAATAAGG
TTCTTATAATCCCATGAGATCAGCTGACAGACTTTTAGGAATGAACACTTGTAGAACAGCTAGGCAGAGA
AGTGCCTTTCAAAATTTATATTACATCTCTACTATATTTCAGACTGTAAAATATTTTTATAAAGTGTTAA
AAGCACAGATAAATAAATTTGAGTTCAAATTAGTATAGTGAAAGGAAGTTTATTTTTCCCAAATATTTCC
TTTACTCAGTATGTGGTCTTAGTCAAATATTAATTTCTTAAATATTGCTTTCCTGTAAATTAGTAGTATT
TACTTCTAAGGACTATATTATATGTTATATATAGAGTATATTATATATTAGGAGATATATATGTATATAC
ATCGTAATTTGTTTTTTCCAAAACAGCCACTTTAGTGTCTTACACATGATAAGTAGATATGAGTAGATGT
TCAATAATGGTATCTAGGCATTTAATAACTTAGAATATTATAATCAAAATGCATTCTTTTTTTTTTTTTT
GAGACCGAGTTTCACTCTTGTTGCCCAGGCTGGAGTGCAATGGTACGATCTTGGCTCACTGCAACTTCCG
CCTCCCAGGTTCAAGCAATTCTCCTGCCTCAGCCTCCCGAGTAGCCGGGATTACAGGCATGAGCCACCTC
GCCTGGCTAATTTTGTATTTTTAGTAGAGATAGGATTTCACCAGGTTGGGCAGGCTGGTCTTGAACTCCT
GAGTTCAGGTGATCTGCCCGCCTAGGCCCCCCAAAGTGCTGGGATTATAGGCGTGAGCCACCATGCCCAG
CCTCTTTTTTTTTTTTTTTTTAATTAAGTTTCAAGGTACATTTGCAGGATGTGCAGGTTTTTTACATAGG
TAAAGGTGTGCCGGTTTCACTCTGTTGCCCAGGCTGGAGTGCAGTGGTACGATGTTGGCTCGCTGCAACC
TCTGCCTCCCAGATTCAAGTGATTCTCCTGCCTCAGCCTCCCAAGTAGCTGGGATTACAGGCATGTACCA
CCATGCCCGGCTAATTTTGCAGTTTTAGTAGAGATGGGGTTTCACCATGTTGGCCAGACTAGTCTCGAAT
TCCTGACCTCAGGTGATCTGCCCACCTCGGCCTTCTAAAGTGTTGGGATTACAGGTGTAAGCCACTGTGC
AAGGCCAGCAATTATGCTGTTACAAGGAAAAATACATACGGAAAAGTTTACATCTTGCTAACATTACTGA
TAATATTAATGTAACAGTCTTATGTATGGACGTCTCCTTAATGCTAACTGAGCAGTTTGTTAAGGGATTT
TCATATGCTCCGTTTAAATCCTCATGACCACCATTTGAGGTGAGAACACTAAGGCTCAGAAATTACCTAA
CCTTTCCAAAGACTCACAATGGTTACTAAACTTGAATAACATACACATTTCTTTTATAGGAAAGAAATCT
TATATATTCCAAGAATACGTTTGCACATACCCCACACAGATATACCTGGTGGTTACGCTACCAAGCATTC
TATCAAATATGTTTAACCCTTAATGGAGCTAAAATAAAATTACATTTGCAATCAAACATACTAAATCATG
TAATTACAGCTAATAGAAAACAACAAAAAAAGAAAGATGGATGACTGTTTCAATTTTCTTCTTGGACACC
AATTCCTGAGGGGTATATTGTGTTAAGTCAAAATAGAGATTTTGGTGGAAGTTTGAAGACATTATGAAAA
GAGTACCTATGCATTTAAAGTGTTTTTGAAGTTGTTTTATAGGTTGAATGACTTCACTTTCCTGGTCTAG
GAGTATTATTGCACTACTGTGACTTGACTTTGCTATGATTCGCATATTTTGTTTTAACATTAAAAAACTG
AAATGGGAAAAAGGCCTTCCCCAAGGTAGAAAAGATTTGAAAAAAGAGGAATGGTGTAATTAAAAGGGAA
ATATGTATAAAATGTCTAGGGTGGGCCAGGCGCGGTGGCTTACGCCCGTAATCCAGCACTTTGGAAGGCT
GAGGCAGGCTGATCACTTGAGGTCAGGAGTTGGAGACCAGCCTGGTCAACATAGTGAAACCCCATCTCTA
CTAAAAAAAATACAAAAACTATCCAGGCATGGTGGCCAGCACCTGTAGTCCCAGCTTCTCGGGAGGCTGA
GGCAGGAGAATTGCCTGAACCTGGGAGATGGAGGTTGCAGTGAGCTGAGATCGAGCCACTGCACTCCTGC
CTGGGCAACAGAGCGAGACTCAGTCTCAAGAAAAAAAATTAAAAAAATGTCTAGGATGAAGTTTATTCTG
AAATGGAGAAGAAACAGGGAATGAAGTTACACACGTTGAGATCCTGGAGCTAGCAGATGGAGAAGTATGA
AAAGCAAAGTGATCTTGTGCAGGATAAGATGATGGTGTTCATTGGAAGTATTTATATATCTTGGAAAATA
AAAATTTAGATATTCTATATTGGAGGAGTACAAATATTTAGAGCATCACAAATTGATACACACAAATGAA
ACCTAAATCACAGTACTTATTGTCATGAAGACTCAATTATCTAAGTCAGTTAAATTGTCTAACATCAGTA
ATGCAAGAAGTTGACTTTTAAGGGTTTTAAAACAGTTTTGGAAAACTAATTTATTTTACATTATGTTTAT
AATAATTTTTGCATGCAATTTTTTTAAATATCTATTATCCACTTAGATACTGTACTTTGGGATATAAAAA
CCATGGGAAATTTGGCCCTTGAGTTCTATGATACGTCTCAATCTATTTTAGTGATTTGTGGATTGGTAGC
TTATCCTCTAGACTAAGTGGTTAAGGTTTTATCAGCACTGCAATGAGATAATCATCTCACACCTGTTAAG
ATGGCTGTTATAAAAAAGTGAAAGGTGTTGTTGAGAATGTGAAGAAAATGAAAGCATTGTCCACTGTTGG
TGGGAATGTATAATAGTGTAGCTGCTATGGAAAATAATATGGAAGTTCCTCAAAAAATTAAAAATAGAAC
TACCATATGATCCAGCAATTGAAATCAGAATCTCAAAGAGATATATGCACTTCTGTGTTTACTGCAGCAT
TATTCACAATAGCAAAAATATGAAAACAACCCAAATGATCATCAACAGATAAAAGAGTAAAGAAAATGTG
GTCTGTATATAAAATAGAATATTATCTAGCCTTAAAAAAAGAAGGAAATCTTACTATTTGTGACAGCATG
CATGGACCTAGAAGAGACATGCTAAGTGAAATAAGTAAAGGGACACGAAAACTTTTGACAGTGACAGATA
TGCTTAACAGGTAGACTGGCAGATAAGAAAAAACAAAGTTAAAGCTGAAATTTCCAACTGATTTATCTTA
GCCTAGGGATTAATCATTAGATCCGGTCTAGTTGGCCTCCAAAATGTGTAGGTCAGGAGTTGGCATCCAA
GTTGGTATTGGTGCCATTGAGGTTTGTAGGGAGGGAAGACCTGGCAGAGATCCCAAGGGCAGTGGTAAGC
GGCTGCTGAGTCCCTCCAGGCCAGTTCCAAGAACTCACAACCTCACTCCTCTCAGCATTTATACTCTTCC
TGGAATTTAAAAGCAATTCCTTCTTTCCTTCCCTCTTTCTTTTTTCCCTCTCTTCCTTCTTCATAAAGTT
TTATTGTAGACATCAGATATAGCAATGAACAACACATGGAGATGTGACGCCGATTTTAAGAGATAATGAA
GAGATAAATATTCGCCACTATCCATCATTGTACCATAAGATATGCTGTGAAAAACAATAAAACAGGACAG
AGAATTGAGAGAAGGAGGTATTATTTAAAGTGGTCTGAAAAATTTTATACAGGGTATTTGAGTAGAGACA
TGAAGTAAATGAAAAACTAAGGGAGCTAGCTAAACAACTGAAGAATCATTCTAGTCACAAAAAACAGCAA
TTGCAAAAGCCCTAAGGCATATTTGATGTATTTAAGGAGAAGCAAGAAAACCAGCATGGTTGAAATACAG
ACAGCAAGATTGAAAGCAGAAGATGAGGGTTTTTCTTGATTTTGTAGGGATTACTAACCTGCGTCGAGGG
CTGCGTGATAAACCAATGCTAAAATTACCAGCATGGGTACAACTTTGGAAAGGGATGAGTGTGCTCTTTT
TTAACTGGATCTCTTTCTGGACATGGGTAACATAAAACCAGAAAAAAATGTTTGAGAAGTTTCTAATTTC
TATACAAAAATAATGTTTTTGTCTTTTTTTGTGATTTAGATGTAAATGTATATGTACATGCATGTATATG
TATTTATGTACTGATTTACTATTTATGTATATGTAAATGTATTTATATTTAAATGTCCTATATTCTCTTC
CTGTCTGTAATGGATTGGATTCTTGGAACCTCTTACTATTTGGGGCAGGGGGCAGGGCTAATAAGACAAA
GATTACAATTGAGAGTAACTTTAAATGTTTGCATTTTGGAAACTTAATGGTGGGAGTACAGTAATATTTT
GTATATTTCGATCAATTTTGATGTTTATTATATGATTATTTCTCATGAAATAGTGGTACTGCATAGATAT
AAATGACTACTGCCTAGCAAAACAGATACCAAGGAGACACCGCATGTTGTCAGGGTTGACCGGTTGCTAA
CTGCTGTGATTTTGTTCTCTTACTGACTCTTTCTGCCTTACGGATCTCAAAACACGCTAGTGAATTTAAA
AATTTATATTTTGGTAAAATATATATAACAAAATTTACCATTTTAACCATTTCAAATGTACAGTTTAGAG
GTATTAAGCACATTCATGTTGTACACGATCACCACCATCCATCTTCAGAACATTTTCATCATTCCAAACA
AAAACTCGGTAACTATTAAACAATGACTCCACACTCCTCTCTTCCCCTATCACCCAGTAACCACTATTCT
ACTTTCTCTATGAATGTGACTATTCTAGGTACCTCATGTAAGTGGAACCCTATAGCATTTGTCTTTTCAT
GTCTGGCTTATTTCACTTAGCATAATGTTTTCAAGGTGTTCATCCGGGTCGTAGCATATATCAGAATTTT
ATTCATTTTTAAGGCTGTGTAGTATTTTATTGTGCATTTATACACATTTTGTTTATCTGTTCATCTGTTG
ATAGACATTTGGGTTGTTTTCACTTTTTGGCTACTGTGATTAGTGCTGCTATGAACATTGGTGTACAGAT
AACTACATGCGTCCCTTCTATTAATTGTTTAGGATGTATACCCTAAAGTAGAATTGCTGGATCATGTGAT
ATTTCTGTTTAATTTTTTGAGGAACCACCACAGTGTTTTCCACAGCAGCTGCACTATTTTATATTCCTAT
CAGCAATGCACAAGGGTTACAATTTCTCCACACCCTTGTCAATGTTTCTTTTCCTTTTTTTTTTTTAAAC
AATAACCATCCGACCAAGTGTGAAGTGATATTTTATTGTGGTTTTGATTTGCATTTCCCAAATGATCGGT
GATGTTGAGCATCTTCCCATGTACTGATTGACTTTCTTTGGAAAAATGTCTTTTCAAATCCTTTGTTCAT
TTTTAATCTGCTTGTTTATTTTTTGTTGAGGAGTAGGAATTTTTTACATATTGCCAATGTTAATCCCTTA
TCAGATATATGATTTGCAATTATTTTCTCCCATCATATGGATTGCCTTTTCACTTTCTAGTAGTGTTCTT
TGATGCACAACAGTTTCAATTTTGACAAAATCCAATTCAGCTCTTTTTTTCTTTTGTTGACCGTGCTTTT
GGTTCCATGTCCATGTGCCAAATCTAATGTCTCGAAGATTTTTCCCTATGATATTTTCTTAGAAATTTAC
AGTTCTAACTCTGACGTTTAGGTCTGTGATTCATTGGTGATTTTTTAAAAATTCCTATATAATTCGTAGA
CTCTAAGAGAGTTTCCAGCTTTCCCATACCCATTGTCCCCAGAGATCTGGTTTTTAGTCTGAATGTGCCT
TACTCTGAACATGTGCCTTCCATTTCAAGAACTCCCATTTCATTTTGTTTTGTTCTGTTTTTTCTTTTAT
ACCAGTATTAAAACTTTTGAGTTGTTGTATAAATATACTAGCAATTTAGGAGAATTCCTGATGGGAAAGA
ATATCCTTGCCTAGCTAGTTAATGAATTTAGGGACCCTTCATTTTCTAATCCTAGATATGATTAAATCAA
ACAAGGATTCATTGTGCTGTAGTTGTAATCCACACAGACCATTTTACAAGGCTTTGCACAGAGAAGGTGC
TTAATACTTGAGTAAACAAATTGAATTAATGAGTAAGAAAGAAAAGCTATCTTGTTTTCTCAACGGTCAA
GTAGATCAATGATTTCTACTATTTAGAATCTTAGATTTATTTTAATAAAGCAAGTTCAAAAATCTCCTCT
CTCTGTTAATTTTTAGTAAAGTCACTTATTTTTCACTCAGTTTTGTTAAAGTGTTTTCAGAAGAGTTTGT
TACCGAAAACTGAAGTTTTAAGGTTAGTGTTTATACCACAGATATTTCTTGTTTTTAGACAATCCTTGTG
GAGGTTAAGTCCCCTAGGCAATACCTTTTCCCACTGGGGAGTTACAAGCAAATGAAAAATGGTGACTATT
TATGGAAACTCCAGGTCACTAAGTAAATTTTGCATTTTCATGAAAATATGACATATGGACATTTAAAAAA
ATGAACAGGTCAATTCATCAGTTAGAATAATAAAAAGAAGTCTGTAAATAAAAGTTAAGCACCTTTGTCA
TTGAATTGTTGTGAACCGTTTTTGCCTATGCACTTGCTTTATCTCAGAATCCAAGCCTCCTGGTCAAAGT
TATATTCCTAATAAATTTAAAAATAATAATTTAATAATTTTAATTCCTTCTGGAATATAAATTTGATCAG
GAGTCTTGGATTCTGAGATAAAGCAAATGTTTAGGCAAAACACTTCCTTCTGGAATATAAATTTGAGGAA
GGATAGGAATTATTTTCTTCCTATTCATTTGTGATCCTGTGATTGTTATCTAATTTGGAATATTCTTCAT
GACTCCAATCCTATTTATATCTGTGAGGTTCTGAAAATTCTGCCCCAAAATGGCAGAAAGTAAGAAATAC
TATCACACATTTAATTCACCCTTAAAAAAATAGCCATACCCTTTATCTTTGAATATATTTTTAACAACAA
CAACAGAAAGAGAATGCATTTTAAACAATATCACATGAGTTTCATCATATTCCCAGATTATGGTGAGGGT
TCCTAGATACCTTGCCATAATCACAGCCTTTAGTTCCAAGAGAGAGCAGAGAAATAAGAAACACAGGAAG
TCATATCTATGAACATGTTAGTTTGCAAAAGGTTTGTTAGTAAACTCATGTTATTTATACCATAGTACTT
TACCATCTCCAGATGAATTTCCACTAAATAAACTGTTCCTTTAGACTCCTCAAACCAGATTACCCCCTTC
CTTTGCTAATATAGATTCTCAAACAATGTCTTCATCAAAAATCTTATGAGTAGATGTCACTAGTTTTGAC
AATGTCCACAAATGTTCCAAAGAATACTTTGGCATGCATGCTTCTTAACTCACTTATTTTGATATTGAAT
TAAAGGGCTTTTAACAATTCCTGCCCCATCAGGAATATTTCATCAGCAGCTGTTACTGTGCTTGGATTTT
GCCTTCTTGAATGAATACCTAGTCTTTTCTCACTTTAGCATTAAGTTCTGGAGCATCCGCTGCTAATGAA
AAATCCTCTGACTCATGCTAGAGCATTTTCATTTTTTTAAAAAAACAATGTTCCTTGTATCTTGCTGGCT
ATTGCCTAAGCTCATTATTTTAAACTTTTGCATAATTCTGCTCAGTCCTAGAGGGTCATCATTACTGTCA
ATGATTAAACTTTTATGCACTTGCCACATTTATTATTTTCTTACTTTCGATCATTTTATTATTTTTCTTT
GGTTCCCATGATATTCCAATGTTTTTGTTATTAAATCTTTCAACATCCTCGGTGTGTTCAGTGTATCCAC
ATTTATCAAACCTGATGGATTTAAGAATTTATATAACCTTGAAAGCAAGGCCCACATCCTGTAGACACTG
AATAAACTAATGAACTTTGTATTGGCAGGACTAAAATGGCTGTGTGTGGCATATATTGCCACCTGTTGGT
AGAAGGCAATGTAGTCTTGGTTTTGCTATAAGCATACATTTTAGTCTTTAAAATATTCTGAGTTGAAAGC
TCCCTAAGAAGAAAAGGTTTTGTATATGCAATACAAAAGCTTTTCTATTATAAAGTAAGCAAATATATTC
TCGTTCCTAAGAACATTAGCTTTAGGTATACAAAATCAACTTAGAAGAAAGTTGCTAAAATCACTAATAA
TAACAGTTACAATGTATTGTGCACTGAAAGTGTAACAGGAAATGTGCTAAGTTATTAAAATTCAGTGTCT
AATTTTTTCCACTAAAATAACCTTAAAGGATTATCATTTTCAGCTCCGGTTTGCTATATAAGAACTGAGC
TTTAGAGAAGTTAATTAAATTCCCCAAGGACATAAGCTAGTAAGCAGTAGAGATGGGATGTGAACCCATG
CCTCTCTCACTCTGGAGCCATGAATTTAACAACTGGGTTATGCTGTCTTACTATTTTACTGCAGATCTAT
ATTTTAAATTAAAGCCATAAACATGTTTGGCAAGATAAAACATAGGTAACAAATATACTCCACATAAGAT
TTTCGTAAGGTTTTGTGTTTATTGGCCAACTCATGGCTTATTATTTAAAAATTTCAGTCTGTATAGGGAA
GAAGGATAATAATCTTGCTGTTATTTATTTAGCCTAGGCACTGTGGTAGGCACTTTACCAAAACCATTTC
TATTCTACATGATAACTTTATAAGACAGATATATTTCTCTCTTTCATTTATGGGGAATTTGCATAATGCT
GCAGAGCTAATTATATGGAATAAACAGGAAATTCAAACCTAAGTCTGCCTGGTGCCAAAGTTATTACTAT
GCACTTTCTCCTAAATCATGTTGATGTTTTGAGGGAAGTGGATACATGTATAATAATATTGGAAATACAT
TTTCTCTCTAATTATTTAAATAAGGGTATAGAACCTGTTCTGCAGCCATAAGTCTGTGATAATTTTGTGC
TCTCAATAAAGTCTTAACTAACAGAGAGATGGGATTTAACGTGAACTAAGAAGTATTTTCTGTCTTCCTT
TATACATGCTGTACAGATAAGACTTTATGAATTTTTTATTAAAACGTTCAGATATGCCAGCAAATGTTGA
CACTAATCTTAAGTTTTCCCAGGGTAATACACTTCTTAAGATTATAAACTTTAATTTCTTTTTTTTTTTT
TCTTTTTTTTTTTTTTTTGAGACAGAGTTTCGCTCTTGTTGCCCAGGCTGGAGTGCAATGGCCCGATCTC
AGCTCACTACAATCCACCTCCCGGGTTCAAGCAGTTCTCCTGCCTCAGCCTCCTGAGTAGCTGGGATTAC
AGGCATGCGCCACCATGACCAGCTAATTTTGTATTTTTAGTAGAGACGGGGTTTCTCCATGTTGGTCAGG
CTGGTCTTGAATTCCTGACCTCAGGTGATCCGCCTGCCTCGGCTTCCCAAAGTGCTGGGATTATAGGCGT
GAGCCACCACGCCCGGCAAACTTTCATTTCTTATAAACTTGAGGAATACTATATTTTAAAAGGCTATACA
TAAGACTAACTTCTATCTTAATAATAAAATAATAAATATAATATAAATGATTATTACAGGGTATTATGAA
ATACCCAAATCACAATATTTTAATAATCATAGAATTTAGAAATAGTCCCTATCAGTTGTCTGAATATAAA
ATTATCGCATATACAGCTTAATTTGTTTTGCATACCAGTTGTTCAACTACTATAAAATGCCATAAAATTG
AATGATTTTAGGACATGAAATATTGAACTACTCTTGTTGAAGTTATTAAACTGATTTGCCATCTACCAAA
TTAAGCATTTCTAAGAAATAATAATTATTTTAAGAATTTATTTACATGATTAACATAATGTTACTTATGA
AGGATGCAATGGTAAGATTTCAGCAATCAGGCAAGTATTTAATGAAAAGTATTTGGTTTCATATATATTG
TATTTGATTTCATATATATTGATACACTTAACATTAGAGGTTCTAAAATGAGTAGCCAGCCTGCCTGTTT
TTCTTGTGATATTATACTTGGCATACTTTGTTTTTATAATCTAAAGAAGAGGTATTAAAAAAAGAGTCAG
AGTGCTTACCATTGGCAGTTCCTTTTAACCTTTTAATTTATAAAGACATTGCAATCCCAATGAAAATAAA
AATAGAAATTTTCCCATTTGGAATTAGATAATCTGGATCTAGTTGGGAAATTGTACACACATAAAAGAGT
AATATCTAATAGCACCAACCCTAATACATAATGTTATTTTATGCAAATAGGGTAATTAAAATAGTATGGT
GACCTACATAGATAGTTCATTGGAAGAAAATACGATATCCAGAAATAGACATAGTTACATATGAGAATTA
TAAAGGTAGCATCTTAAATAGGTTGAAAGAAATATTATTTAATAGTTATCATTGGGACACCTGGGTAGTC
ATGTGGATAAAAATAAATTCAGATTACTGCTTTGCCACCTAACCCAAGATACATTTTAGTGGAATCAAAT
ATTTAAAATAACATACAAACCACAAAACTGCTAGAAGAAAACATGTGAAGGTTTTGTTTTCTTTTTTCTT
TTCACCTGAGATGGATAATATATCAATGTCATTACAAGGTTTGAGGGAGGAATATCTCACATATGCATGT
GAAAACCCAGTCATCACACTTATGAAGTACTTGAAGTAGAAATTATTTTCTAAGATAATATAAAACACAG
GAGCCACTGGAGAAAGGATTGGTGAATTCAATTTAATTAAAATGTAGATGCTTTTAAAAGATAAACTACC
ATAAACCAAAAGAAACAAAAACAGCAAACTTACAAAAATATTTGTATTGTATACCACAGAAAAAAAGCTA
ATTTTATTATTTTGTTAAGAGTTCCTAAATTAATAAAACACTGATAGTCTATTAAAAAAGTGGACAAGAC
TACATACAGACAGAGCACAGAAAAAAAAGCACAAATGGCATCTGAGGATATGAAAAGATCCCTGACCTCA
CTCACAAAAGAAAATGCAAACTAAAATGATAAGAATTAAGAAGTTTTCGGCCGGGTGTGGTGGCTCATGT
TTGTGATCCCAGCACTTTGGGAAGCTGAGATGGGTGGATCACATGAGGTCAGGAGTTGGAGACCAGCCTG
GCCAACATGGTGAAAACCTGTCTCTACTAAAGGTACAAAAATTAGCCGGGTGTGGTGGTGGGTACCTCTA
ATCCGAGCTACTCAGGAGGCTGAGGCAGGAGAACTGCTTGAACCCAGGAGGCAGATGTTGCAGTGAGCTG
AGATCGCACCAAGGCACTCCAGCCTGGGCAATGAGAGAGCGACTCCATCTCAAAATAAATAAATAAATAA
ATAAATAAATAAATAAATAAATAAATAAATAAAAGTTTTATTCCCCACTTATTAGGCTCACAAAGATGAA
AAAAAATTTATGAAATACTTTATTGAAGGTATGTGCAAACAATCCCTGACACATTGTTTGGGGAGAGCAT
GGATTGATAGGCCTTTATGAAGGAGTTACCAATATACCAATAACCTCCTTGGTAGTTTCAAAAGGTGGCA
TCTTTCCCTGTTTGGTATTGGAGACCCAATACAAGAAATGGGAAAGTGAGCATCAAGCAATGCAAGCTTT
ATTCGATGGCCAAAGAATGGAGAAGTGCAAGTATGGCGCACAAGCTAACTTCTTGACTAGTGAGGAGTGA
GGAAATTGAAATAGAAAGTATATCTAATGAAGAAGTTGGTCATTAAGAGTGAGGGGAGGAATGTTCATGT
CTTTTCCAGAAATAGGTGGTGAACTTCTTGGAACCAGAGTGCCGCCTTCCTTTTTGTCCTTTTATGGCTT
CTTCTGGTCATTGTCATGGGGATTATCAACTGTCATGGCACTGGTGGGGGTGTCATTTAGCATGAAAATG
AGATTATAATGATGCCTGAGGTCTTCTTGAAGTCCTTTAGTCAACTATCTTGGTTCTAAGCAATCTCAGC
TGCTCTGGTTACAAATGGAACATTTTACCTGTTTCTTAAAGATCAGCAGAGTTAGGGTAGGATAGAAATT
CAGCTATGGCCCTGTAGGCGTTACACTAGGTAACAAAAGAGAATTCGGCAATATTTTATTATGGCAAATT
AAAAACGCACATACAATTATGACTCAGCAATTTCACTCTGAAGCATTTGTCTTACAAATAAAATACCGTA
CTGTGAAACTATTCAAGAGAATGTCCTCACTTATAAGTGAGAGCTAAATGATGAGAACACATGGACACAT
AGGTGGGAACAACACACACTGGGGCCCATTGGAGGGTGGAGGGTGGCAGGAGGGAGGGGATCAGGAAAAA
TAACTAATAAGTACTTGGCTTAAGACCTTGGTGATGAAATAAGATGTAGAACAAAACCCTGTAACACAAG
TTTCCCTGTGTAACAAACCTGTGCATGTACCCCTGAACTTAAAATAAAAGTTACAAAAAGAGCCCACAAA
AGAGTAGGTTAAAAAAAGAAAAAAACTAATGAGAAGATAATTATTGTAGCTTTGGGAAACAACCTAAATG
TTCTACAGTGTGGGAATGACTAAATAATTTGGGTACAGCTGTACAATAGGACTTTATGAAGCTGCTATAA
AAAGAAGTAGCTTTATACATGCTGATACAGAAAGATTTCCAAGTCTGATTTTTAAGTGACGAAGGAAGGT
ATGAAACAATGTTTATATAGGGTATTATTTGCATGAAAAGAAAAATGAATAGATGTATACATGCTGATAA
AACCACATGGATGGATACACAAGGGGTTGGTACTGTGGTTGTCCACAGGGGAGGAATTTGGATGGCTGGG
AAACATTGAAGAGTGAGAGAATTATTTTTCGTTATACATACTTTTGAGTTTTGTGGCTAAATTTATTCTT
CAAAAATAAATATAAGAATGCTATTTATGGGGAAAAAATGGGAGTCATAAACCTGCAATTTGGACCTAAC
TTTTCAACTTAAAAATGTTCTGTTCTTGGAGTTACTAACTGCTCTGATGCTTAGGATGATAATCAAATAA
TAGTTCCTCTAAAATATGTCTGGAATGAACTTGAATGAATGAATGAATGAATGTTTTTTGCAAACCATTA
TGTGTGTAAAAATGTAGAGTATTAATATAAGGTTTATTTAGAATAGAGTCATCTGCATTAAAGAAACTTC
GTTATACAAAAATCTAAATAAAAAAATGTTGTACAGTAGCTCTGAGGAGGTGGATGACCCAAACACTGAG
AAAAGAAAAGAGGCAGAGGCTGAGCAGGATAGTTCAGTTTGAGGTAAAGAAGCAGCCTCACCCAGCCTCA
GACAAGACTCCAGGGACTGTGTGAAAAACCTCATTTTGGCCAGGCGCAGTGGCTCACGCCTGTAATCCCA
GCATTTTGGGAGGCCAAGGCGGGCAGATCACAAGGTCAAGAGATCAAGACCATCCTGGCCAACATGGTGA
AACCCTGTCGCTACTAAAAATACAAAAATTAGCTGGGCGTGGTGGCAGGCACCTATAGTCCCAGCTACTC
AGGAGGCTGAGGCAGGAGAATCACTTGAATCCAGGAGGCAGAGGTTATAGTGAGCTGAGATCATGCCACT
GCACTCCAGACTGAGAATCTGTCAAAAAAAAAAAAAAATCTCATTTCTAGGCCTATGTTTCCCAGAAATA
TGTCATGAAAATAAAACTGCCCTCCCAATGGGAAAACATATATATGCACAAAAAGGTAAATATTTTTAAT
GTTAAAGTTACTGCATAATTCCTTTAAAGAGTAATTATATATTTCTATTCATTTTTGAAAACCAGTTTTA
GTAAAGACAAAGTTAAAAGATGACAAACTGGCAAAAGAAAAAAAAAAGAAATTTTTGTGAAAAGGTATCA
ATATCCTTAGCGTGTGCACACACACCCACACACACACACAATCCCTTGGCAAACAAGAAAATAATGAATA
TTTCAATAGAAAAAAGATCAAGAGACTAAAGACACTGACCAAAGAAGAACTAGAAATGTTTCACGAAATT
CACATTTTAGATAAAAACGTTTTAAATATCAAATTATGAAGTTTTTTTTTAAATGACTATACTTAACATT
GTGGGAAAATATATGCTTACATGTTGCTAAAAATGATATACATTTTTATAGCCTTTCTGGAAACATTTTG
TCAGTATGTATATATATATAAAAGAAGACAGGTTGGGTGTGGTGGCTCATGCCTGTTATCCCAGTGCTGC
CAAGAGGCAGAGGTTGCAGTGATGGAGTGAGCCGAGATCATATCACTGCACTCCAGCCTGGGTGACAGAG
AGAGACTCCATTAAAAAAAAAAATTAGCCAGACATAGTGGTGCACACCTGTAGTCCTAGCTACTTGGGAG
GCTGAGGTGGGAAGAACACTTGAGCCCATGAGGTTTGGAGGTTGCAGTCAGCTATGATTGCACCACAGCC
TGGGTAACAGTAGGAGACATTGTCTCTTTAAAAAAAAAAATCAGTAATTTTCTTGGAATAGGTTAATTTC
ATTTATTTTTTTCTATGCTTTTCAGTTTTCTAAATTTTCTCCCAATGAAAATTTGCTATATTTATATTCA
ACCATATTAATAACCCCAAATTAGTCACATGTGCAACTTTCTGCAAATAAATGTGTCTTGGATTTAATGG
AAATGAAATCTTTTCAGTATAGCTTCTCCCTGTTATTTGCGTACAAGCTTCAAACTGCATAAACTATTTT
AATGGCAAAAATTAAGTTAGGGTTAATCAACCATCTAGGATTGTATCTGTGCCTAGGTTTCTAAAACAAT
TAAATACTTGATCGCTTTGTTGGATGTTCAAAGATTTGGCAATTTGTCCTTAAATATATGTTTCAAATAA
AATATCTACAATTTTAAGGTGCTTAAGTACTGGTGAACATGTCAAAATGTGCCTAATGTATTTTTAAGTT
CTGCTGTGAACTTAAAAACTTTGGGATTTGTGTAATCCCAAATTAGAGAATTTTAAACTTTAGTTGGTGT
CCACCATATTGTCCCCCTTTTCATCCATCTGACCAAACTTTTAACCTTCCTGTTTATGGCTGGGGAAAAA
AAAAGCTACTTTAATTAAACCTAAGCAGATAGTGTTGTTTTTAAATTGTATTTCGATGTCACTAAAATGG
GGAAACAGGTTGTAATAGCAGAATCCTTATATTTTTTGCACATATCAGCCTCCATAAAAGGATGGAAATT
GCTTTGTTTTTCAATGTTTTAATTTTTTTAATGAGGAGAAAAAGAGCAATTGAGAGTGCCATAAATTAAA
GATAGCGTTTCTGATGTAAAATCAATGATAGTTATGGATTATTGTAAATGTTTGAAGATGCTCTTGGAAC
ACTGAAAAATCTTAGAAAATTTTGTTAAATAATAATTAAAATTGTCTTAAATTAGTGTGACATCTATGTT
AAAGGCAATATAATTAGATAGCGTGCTTCTTCAAGTGCTTGGGTTAGGAGAAGTATTTCACCATCTTCAT
TCATCAAGCATTTAACAATTTTATATGATGTGCCAGATATCATGTTATACCTTGGTGATAATACATGCCT
AAGACAAATTTCCTGTCTATGAAGTTTGGAATCTAGTTGAAGGAGACAGGCGTGATGAAAGAAATAAAGC
ACTGTAGAAGTAGGATAAAAATCCGCATTTAGGGGACTGCTGGCAGCTCAAAGCAGACTGGAGATATGGG
AGAAAGAGAAGCTGATGAAGGGTTGAATTTATATTTGAGGGAAGGAAGGAGGCGAAGTCAGATCTTTTCA
TACAAGAGGTATTGAGCCAAATATTACAAAGCAAGTAATCAGGTGATCAGGATAGTTGAAACTGTTCAGA
TAAAGGGAACATTATGAGGATGCAGAAATTATCTAGCTGACCTTTGGGGAAATTAAACGTAGAATAATGT
AGCTGGAATGTAGACTGCAAATAGGGAAACAGAAGAGGCTGTCAGCATCAGGTTAGTAAAACCTTCTATG
ACAGACCAAAGACATGACAGGTTATTCTGTACATAGTGGGGAGCTGTAGATTATAATTTTTTTTTAGTCA
TGGGAAGATTATCAAATTTCCTATTGCAGACAAAGGGTCTCTTGTTCATTGAATGAATATTACATAATTT
ATTCAAACTTGCTTGGTGTTAGGCAACTAAATGTTTTAAGGCATTTTTTTGTAGCACAGCTCTTTTTTCT
CTTTTTTTTTTTTTTGGAAACAGGGACTCACTATTTTTGTATTTTTGAAGGGAAGGGGTTTCATCATGTT
GGTCAGGCTGGACTTGAACTCCTGACCTCAAGTGATCTGCCCACTTCGGCCTCCCAAAGTGCTGGGATTA
CAGACATTAGCCACCACGCCCAGAAAGCACAGATCTTTTTTCAAAAGAAATTTGTGATGTTTTAATAAGA
AAGAAATTAAAAAAAAAACAGATACAATGGAAAGATATCGATCCTGGAACTTTTAAATCTTGGTTTAGAG
AAAACAGTTTATTATTTGAACAACTTCAAACACTCAAGTTAAAAAGCACTAACAAGGCACATCTCACTTG
CACAAAAATGATACCAATTATCAATTGTTGGTGGCAACAGAATGGAGCAGCACAGAAATCTGGAGACCCG
TTTTCAGTTGAATGCTCATAGTTCTAAAATAGGCTCTGATTTGGAATTCCTTGTCTTACTTTTCAGTTTG
ATTAACGTGGAAGTCTCAATATCTTCTTATGCTGTGTGTAGGAAAAGTATCTTGATCAGTATATAGAAGA
ATATCCCACAATGTTACTGCAACTAAGAAAAAATTAACCCATTGGAGAAAACATTTTTAATCAGTTCATT
AAGAGTCTAATGAAGGTGCATATACGAAAAGCAAGATGTACTTTAATTAATGATTGATGTTTTGTGATTG
AAGCTTGCTTTCCTAAATAATATTTCTATACTTCAGTACAGAATGTAAGGTCCTTGCTAATGCCTTATTT
GGTTTCTTAGTTTCAGTCTTACTGCAGTCTGCTTTTATTGTAGGCTTATTACATTTATCACCATCATTAA
TTTACCATGGTGTTTTATCTTTTTAACTTGTTATTTAGATCACATTGTATAATGCTTTATTTCTGGTCTT
GTAGCAAAGGAATGCATACAACAAGCATGTAAAACTTTATTGAATAGAACAGTCTTTAAAGGGGAATCAT
CCATAAGTCAGATAAATCCATGAGTAGTATAGATTTATCTACTGTAAATTTTCTGTCAGGCTGTTAAAAG
TAGGGCCGTGTAAATCTTACTGTCATTCACATTATAATAGGCACACTTACCAATATACCAACTTTATAGT
TTTTTCTCCCCATTTTACGGGTTTAGGGCTTATTTTTTTTGAGATGTCAAGTACTATTTTAACATTGTAC
GTGTTTCTTAAGAAACGCCATAGAATGAAGACAATAGGGAAGTCTATTTGTGATATATTCTAGCTCACTT
ACATTTTTTACTGTATCTTACAACAGTAAACATATCACACTATACTTGAATTTAAATTTTTTCAAGTATC
ATTGCATTATTTTGCACATAAACATATTCTTAGATGGTATCAGGTTGATGATGATGAGCTTCATGGTAAT
TATTTAAAACTCCATAATGAGATTTGTTTCCAATCTAATCAAGAAGGGTAAGAATTTGGGGAAAAAAAAT
CTGTTCTATAGTAATTAAAGTTAGCCATATGGGCACTTGGCTCTAAAGACTTGGATGTAGTGAATCTACC
CAACCCCCAAGACTAATGACTTGGATGGTCTTACTGTATTCTCTTGTCCATAGCAAAGACTGATGAATCC
AGCCAGGGAATTCTCCACCAGCTCTGCTTCTCTGAAACCACAAAGTCAGGAAATGTAATCAGAGTGCTCA
GCTGAACCAGAGCAAGAGCTTTCCTAATGGCTAACTGTGACTTTAGAAATCACATCCATTAGATAAAGTG
ATTAAATATACATCAGTCTTTTGGCAAGAGAGCCATGAAGATATTGCAATAGAACCTTACCACATGACCC
ACATCTTTTGCTTGAAAAAGAAACATGTTTTCACCTTTGACACATATCATCCCTAAATTCCTCAGCTCAG
TTGCAGGAAGCAATTTTTTCATGATAAGTTGTATGCAGTTCATAATTTTATATAAACCATAAAGTAATTG
TGTGGTTGATAAACTTTATAAGGAAAATGTTTCTGACTTTCTCATTTCACCACCCTGATGTGTGGGTTTT
TGTTTTGTATTATTCTTCTTTTTAGGCTTTTCATGCTTCAAGACTTCAAGATATTTTGTTAAATTCTTCC
CTTGGAAGCCTTTGTGTCGCCAAATCAGATTTTTTTAAAACTTAGATATGAATGTCTTAAGTGTCTACTT
AGGAAATATGTGTGTATGTCTGTGTGTGCGTCTGTGCTTATATATAGAGAGCAGGCAAATTACTCTTAGC
ACTCTGAAAAAGCTTTTTTTGTTAAAATAATGTTTTAAAAATATTATAAAAAAAGTCACCGTCAAAATAG
CATTCGCTTTATATTTGTAGTTGAAATAATTCCTGTTTCTTACTCTTAGTTATAACCATCTTTTGATTAA
TAATATACTGCCCTTCACATTTTAATACACATGCAACTAAGCCGCTGATGTAATACATATACTTAAAATG
TTCTTTTTGAGCTTTGAATAACCTTTTGCCTTTTCCCTGGTGATCACCAGTGGGCAAGATGATTGACAGA
GAATGTGTTTACAAACATGGAATCATTGATGACTAAGTTAAAAGTGACCTCATAAAATATAGTTCTAATT
CTTATTTACAGACAAAATTCTAAGAGCTCAATTCAATGTGTCTAGGATAATAGTGGGCATAGTTAAATAA
ATAATAGGAAGTGATTATCATTTACAAAATTTCATTAGTGAATTACTAAAGTATACCCCTATGCATACTA
ATTCCTCTTCCCCAAATCAAAACTTCTCATATTCTTAAATTTCTGAATTCCTAAAATCAGCAGAGATGAA
GGGATGGACCAGCAACTATAATTTTGTTGTGTTTTGTGTTTCTGAGGCACATATAGGGATACCTAGTTTC
ATGAAACCAAGTGGTGGGGACATTACACACAAAGAGAAAGCTGTTAAAGGCAGAATGCCCAGACAAGAAT
GAAAATGAAATTCCTGGAGGGGCATCCCCATAGTTTTTCTGATAATCTCTCTCCTTACATAAAAAGCTTG
AATTACTGGTAAATTATCCATGCAGGAATAATAGAATCATAGAATTTTTGTAGTATAAGGAATTTTATTG
ACATCATAGTTGAAAGCAGTCATTTTGTAAATGTTTTTGGTGCCCAGAGATACTTTAATTATGGCAGAGG
AAGTTATAGACAAGTCCACGAACCAGGAGTAGAAAAGTTTTCAAATGAGAAATATTGCAGACCAAGCATG
TTAATTTTATTTTATTTTTTATTTTTTATTTTTTATTTTTTTTGTGAGAGGGAGTTTCACTCTTGTTGCC
CAGGCTGGAGTGCAATGGCGCAATCTTGGCTCGCCACAACCTCCGCCTCCCAGGTTCAAGCAATTCTTCT
GCCTCAGCCTCCTGAGTAGCTGGGATTACAGGCATGCCCCACCACGCCTGGCTAATTTTGTATTTTTAGT
AGAGACAGGGTTTCTCCATGTTGGTCAGACTGGTCTCGAACTCCCGACTTCAGGTGATCCACCTGCCTCG
GCCTTCCAAAGTGCTGGGATTACAGGCGTGAGCCACCATGCCCGGTCTGAGGTATTGTTATATACACGGC
AACTCTAGCCTTATAATGCATTGCCCTTATAGTCTACTTAGGGTTTTTAGGAATATTGTGACCTAATTTA
AAAGTTTGTAGAGAGATTCTAATTTTCAGAATTAACAGTACTTTCATTTTCTCTGACCATTGAAATACTC
CTAAATTGCAAAATGAAGTGGAAGTACACAGTAGATTAGTAAAACATATGGTTCAATGCATAAATTGAAA
TAAATAAAATGAGACTATTTAGAAAATGATAACAAATACAACAGTAAAAAGAACTCATTACATAACTTTG
TGACTTTTTTCTTTTTCTTTCATTGGTTCCTTACATTTTGAACTCTTTCTCACTTTTTCTGTTTCCTTCT
TTTTAAAGTAAGGAATAGAGAAAGAGTGTATACAACTGAATAAGTATTTATTGATTACCTTCTATGTACC
CCATTGGGCTAAATATGGGAGTAGAAAGATGAATAAGATGGTCACAGCTTGGGGGAGGGTGATTATGTCA
AATCATGATGAGAGGAGTTTTTTGTTTGTTTGTTTGTTTGTTTGTTTGTTTTGACGGAGTTTTGCTCTTG
TTGCCCATCCTGAAGTGCAATGGCGTGATCTCGGCTCACCGCAATCTCCGCTCCTGGGTTCAAGCGATTC
TCCTGCCTCAGCCTTCCGAGGAGCCGGGATTATAAGCACGCGCCACCACGCCCGGCTAATTTTGTATTTT
TTTTAGTAGAGATGGAGTTTCTCCATGTTGGTCAGGCTGGTCTTGAACTTCCGACCTCAAGTGATCTGCC
CACCTCTGCCTCCCAAAGTGTTGGGATTACAGACGTGAGCCACTGCACCCGGCCACTACTTTGGAGGTTT
AAGAAAAGTAGTTGTAGGTTTAAAACTCTTGGACTGGCTCCTGAGGGAAGAATTAAAATGCCCTATTGGA
GCATAGCCACCTATCTAGGATCTTGAGTCCTAGATAGGTGGGTGGGAATGAGTATCAGGAAATTGGGCTG
GGACCACTTAGGACAGGCCCTTGTGGGTTAATCCCATAAGCTATTGAGAAGCCACTGATGATTTTTTAAC
AGAAGAGAGATGTGATCAATTTTTAATTACCACTCTGACAGTACTATATATTCTGTGGATAAGACAAAAT
GAGAGTGGAAACCTAGAGACTTGGAGACCATTGTGATAGTACAGATAAAAGATAAAGAGAAAAAGCAAAG
GGAAAGGGTTTAGGGTTGATTTAGAAAGTATAATCAATAGCCCTTGTTGATTAGATGTTACTCTATTCTG
AGTTCCCAGGGAGTAAATGGTACTTTTTATTACTAGCACTGGCTACTGACTGGCAATCATTTAAATGAGC
GGGTTAAAATAAGAGTATATGAAAAATTAGAGTTTGTGGAACTTGAGGGGCATATCTAGGCTGGAAAATA
GCTACTTGTCATTTTTTTTTTTTAATGTTGGAAGACTTTCTATTCTTAGTTTGTAGGAAAAATGTACATG
ATAATCAACAAACCGTTACACATGTGCTAAGGTGGTATATTAACTAATTTTCATTGGCTCATGAGTTACA
TGCTACCTGGCATCCTCTTGCCTCTTCCTCTCTGGAGCCACAGATTGTTAGCAGAGAAGCTGGAAGAACC
TACCATCTTGCCATAGGAAAAAGGTTTGGTCCTTCTGGAACATGCCTGTGAAAAGTTGCCAATTTCCTAT
GTAGGTCCTGGACTTATCTGGAATTCAAATATGACTAGACACCATGTATTTTATCTGGCACTCCGATCCT
ATGTGTTTTTTTTTAAATATTTATCCTCCTTTGTCTCTTCATGACCTTATTATACAAGTTCTAGATCAGA
GTGTAACAGCCTCTTTAAATTCTGTGGCATGAACTTTAAATGCCTGTGAAGAATGGGGCTGGGTTACGAT
TGGCTACCAGTGAATTCCAGGTAATTTAGACACTTCCTGGATTTGTTTTCCGTGTCATCATTTATTGATT
ACTTCATATAAAGTCTTAATGTGTTTATTACTCAGGAAAAATGATGGAATAGTAGCAGCAATGCCACAGG
TGTGTTGTGAACATTAATTAGTTAATTACAAAGCATTTAGTTGAGAGGCATTCTAAATAGCACAGAACTC
AAGGCTTTCCTCCCCAACCAGGGGTCCACTTAAATAGCTCTTTACTAAGTCAGGAGAAACAAACACATCT
TGTACATGAGGTACATCAACCACTAGGCATGTTGATCTCCCAGGGTGATTTTCTGCATGAAAACCTATCC
CCTGGGTGTCAAGAGCTTTCTAAGAGCAATGATTCATGAACCCTCTGGATTTAGGACAATGCTATGAAAT
TATTTTCATCTGTATTCTGAAATAATTACTTGATGTCCTATTAAAGTATTTTATATTTTTTGCCAGAAGT
TGTAGGTGTAATTGGAAACAACAGAATACTCAAAAAAATAAAAGATCCCAAAGGAGAATAAGAAGAAAGA
GAGAGAGGAATGGGCTATAAAGACGGCAAGAGTGATGGACCAGTGCCTAAAGATTTTAGACAGACTCTCA
AATTTACTCTTCTTATGTTCTCTCTAAACACCATTCTTATTAGTTCAGCATCACTGTAATAACTAAAAAT
ATTTTAAAAAGTGGACAAGGCAGATGTCAGCAAATTGATGTGGAGAAATGGGACAGACACATGGATGACT
CACTCCCAGTCCCAATTCCCTATTTTATTTTTTCATTTGAAATATCCATCTCTTCAGAGTTCTGTCTAGT
GTGAGGTTCAAACAATTTAAATGCTTTTCTCAAAACTATTGTGCAAATGTCTGTTTTTCTGTATTTTTGT
GTCTTTCTGATGTTCCTCTCCTGGCTTTTCACCTACTATCACAAATTACATACTTTATTATGTACTTTTA
TATATCATATACTTTTCAATGAGCTTGATTCCCACCTGAACCCACGCTTTAGAGAGAACAATGATAGGTT
TCTCTGCACCTGCTCCATATTTAGTAAGTTTCCAATTCCTTTTGGTGTCAAACAATAGCAGGTATCTCTG
CAGTCCCTTAGGTCACTAATGTGCTTGGTTTTGTTGTCAGGGTAGCAGCTGAACCATATCTTTCACAAAC
AGGAGATACATATTTTTTCCCAGCTTGAGCAGAGCATCTCACATTTATTAGGAGAAATGCCCCTAGAGGC
CCATCTGTGTGTCTTTGACGGCTCAAGTATTATGTACTTTAAGAAGTTTAATACATTTTCTACTGGACAG
TATTGAGTTGTTTTAAAATATTAAAATCTTACTTTGCTGAGATAAATAAAAGGTATTCAGGCTTTTCCAA
GACAAGTATGAGAAATGCCAAGTTACAACAGGTTCACTAATGAGGGAGAAGAAAAGTTTCATGATAATAC
TATGATCTTTGCTATTGCATTTGGAATTTAGTTAGACCTAAATAATGAGAAGACTTTTGTGATGATCTAT
TCAATTTTTATGGCCTATGGTCTTTTTGAGAAATTGATTAAGGCAGGGGAAAGGGGATAGTGGTAGGGCA
TCACTGACTCTTGTTTTCTTGCTTGTTTACTCTTTTCACGAAGTGGTAATACAAATTACCTCCCACAGCA
GCACAGGTTACTCAAAAGCCCAGAGGAGCAGGAGAATGAGACATTCCTTATTCATATTTCAGTAAGGTAT
TCTTCTCCATCTTTGTTGTTTCAAGGCAGATGACAAAATTTAGTAGCCATTTTAGTGCAGAATACTCCCA
AATTCAGGGAGGAAGCTAGATTCAGTTGTTTTTCAGTAATATTGGAAAGTGTAATGTGTAATGCCCATGT
TTAAACAGTTGCTTTCTTCCTAACATGATAAACTTCTATCGTTAGAAGATATTCTCTTGGGATGAAAATA
AAAAGATATAGCATAGGCCCCGTTTATTTTATTTGGGGAGGAGGTAGTAGTAGCGGAAAATATAACCAGC
GATACCATCTTCATTACTAATGATGACAACTTACAACATCAGGAGCTCAGGAAGAAGGAAAGCAATAAAT
ACCTTCATCTTCAACAGGATTTTCTGGCAATTATATCTGTTTTTGTTTGTATGTTTGTTTGTTTTGTCCT
CTATTAATTTGAAATGCCTAGATTGTTTTTCTGGAACTTAGTTTTATATCAATCAATAAACCCCTTTTCC
CTACTTTTTTTTGTTACTTTTTTTCAAGAGCATACTGACCTCAACGTGGTGAAAGTTTTCAAAAGAGGCG
GATATAAGCTTAAAACCTGCCTCTGCCCACTCCTGATCTATCTCAAATGAACATCTGTAACTCTCCATGA
ATTAAAATGAGGATAATAACATGTTCTAATTGTTATAGAAAAAATTACATTTGTAGCACAGGGTAGCTGC
TTTATGCATATTTTTTTCTAAGTTATGCACACCACAATTCAGTACTAATTTTGTCATGATGTCCTGAAGA
AATCCTAAAAATAGAGATTCTGTGAAAGAACATTGAGTACCACATAATCAAGATATATATGTCTTGATTC
ATTGATTGATTCATTCATTCAGGGCATTATATAGCAGTCATTTTTTTGCTACCCTTCCACATGGATGATA
GTTAAAGACTCTACTGAAGCCACTAGATACCCATCTTATACACAGGCTGTAATATTTTAACTCCTGGAGT
AATTGGGGTATATTCACAATAAAATCAGCTCAAATCTAATTTATTTCTTTCATGAGGTCTGAAATGTAGT
CAAAGCTTCCATTTAGTTCCTCGAGAAAAAGCTTCTGGCAAAGATAACAAAGGACATGATTCAAATGCTT
TCTGTCTTACACTGCTGCTTCACATCAGGGGCAGAGGATTTTATGTTTCATGGCCTTCTCCTCCCACATA
TTTTGGGTTTTCTCATCTAATCATACAGCTATGATGTCAGTGATGGAAATTTCCACCTCTTAGTTTGGTA
TCTTGATTGTTTTTTCTTTCCCTGTTTGCACAAAGGATTGTAGGTGGCTTTCCAGAAAAGAGACATGAAA
AGGATCACTAAAAGGAGCTTCAAAACAAATTAGATTATTTGTTGTGGGGAGAAAAATCATAGCAGCAGGA
AATTTCATTGTGCCTTTTACACAAGATCTAACATGCTGATGAATTGCTAGTATAATGATGATTTATAGAG
CTGAGTTTCTTTTGACCAAGCCCAGTACTCTTGCAAGGTTAAACAATTCTAATGGCGGATGACTTGAAGG
ACTATTTTCTAAGCGAATGTACAAATTACAAGTCCTTAGTTTCGTAAGATGCTGTTTCATCAAAGGCTTT
TCTTTGTGGTTTCTCATATTTCATATTCATGTTGACTCCCAGGTGATAGTTAATGGTATCTTTTTTAGTA
GTGCTATTATCAGCTTCAAATGAAATAATAGATAATAAAAAGCCTTATTATTAATGTCATATTTTGATAT
ATTTTTAACTTTATAAGGCTTTTTAGTATTATTAATGTCATATTTTGATATACTTTTAACCTTATACTTC
ATTTATTCTAAGTAATGTGATGTGATCAATAATTTTTCAATTAAGTACTTTTCTTTTTGTCTGTGCTAGT
TTAGTTTAGTTGATTGGAATATGATGTTACTGTAACTAAAGCAAAGTTTGGATTCCTGTAAAAAGACTTA
GCATTTCTCCATAGAAAGGCATATGTACTTTTAGATAATTCTCCATGATCCTAATCTGTTATCTCACAAG
TAAAGAATTGACACTTAGCATTTTGAGATTTAATATGTATTGAGTGGTTCTTGTGCTTTGCCCCTCTACT
CACTTTACATTATAAAGCATCTACATAAAATGATTTGTACATTATCTATTGTAATGCACACAATAATATA
ATGAAGATGGGGATAATTGCTATCTCCACTTTTTCTTTTCTTTTCTTTTCTTTTCTTTTTTTGAAACACA
ATCTCACTCTGTCTCCCAGGCTGAGTGCAGTGGCACAGTCACAGTTCACTGCAACCTCCATCCCCAGGGG
CTCAAGCAAACATCCTGTCTCAGCCTCTTGAGTAGCTGAGATCCACTCCCCTCGGCCTCCCAAAGTGCTG
GGATTATAGGCATGAGCCATCACACCTGGCCTTCCACTTTTTCAATGAGGAAACCGTGGTTTAGAGAAGT
AAAGACATGTTATCACATGCAACAAGAAAAAAACAGAACTGCAATTCAAATCAGGTTCTTTCTCATAGTT
CTTAATACCATCCTATAGTCCTCAAACCAAAGGTTCCCTCTTGCTTGACTTTGAGATGATGTATAGAATC
ACTCTTGAAATAAAATAAATATTTTATTTAATTTTTTTCTTTTTCCCAGAAAAGTAATCTAGTATTTTGT
ATCAATCAAAAACTTCTATTTTGAAAATACAAGCATTCCTAGTTCTATAAAATATCAACATATGTAAAGT
CTCCTAAAAGCTTTGGGTTTTTATATGTGAAGCCTTTACTTCTGTTTTCTTTTTGAAATAAAACATTGAT
TAGAATTCTAAATAGGTTATCACAAATCTATAATTTTTTTCCTATAAAACAAGCAAAGTATATGACAAAA
AAATCGGTGCTCTTTTTTCACCTCACCAAGAAAATTAGTGTAATTATTTCATATGAATAAGACTTTTTAC
CTGCACTACATTATTTAGTTAATTTTACAGGCTCTAGAAAGATGTTATGCAAGAGAAGACATTTCAATTA
ATCTGATGTTGAAAGAATGCTAGTTTTAAAACTCAATGCACTCTATGTGCTAAAAACGTTTTTGCAGCAT
GAAGGTAGGGATGGACAGGGACATGTTTGCCTGAGTTCTCCAGGGAAGCTTTGGTGGTGGATGTATGCAA
AGGGCATAATCTGTGGTTTCCAAAGTTGAATATGCACACCTAAGAACAATGCTGGAAGAGCTTTGTCTTC
ATTCTTATCTTTAAAAGTTTCAATTTTATTCAAGATCAAGGTAATGATAGATTTAAAAAATAATGATAAA
ATAAAAAATAGTTTCAATTTTGTGTATATTTTTAAATGCATATGTTTTACACTGCATGTGTAGATTGTTT
ATAATTTAGCAAGCATATAAATTCATTTATTGGGAGATCTTTTATTATTATTGGTATTGCTATTATTGAG
CTGGTAAGGGTGATGATCAAAAATAATTGGAGACTCTATTCTAAACGTGAGGTAGAAAATTTTCTCTGAC
TCTAATGGTTTAATCTTTAAAAAACAGTTTAAGCCACACTATAATTAAAGCCATAATGTTTGTAACACTT
AATTATAAGGATGGAAAACTTGGTCCTATAGATATTATCACCTTAAGGAAAGGCAGGAAAGCTAACTGCA
GTAGTTGACCATTGGCACATTGAAGGGGTCATGACTGGCCTGCAGTGTCAAAAAGTGACCAAACAGGGAC
ATCTCCATATGGCATTCATTGACTAGATTGGTTCTATTACTGATTGAAGTCATTTGTGTTTTTTGTACAA
ACTTTAAATAGCCCAAAGATTCTAAACGATTTTAAAGTAAATGCAATATATTATTAGTATTATATAGTCT
ATACATACGTGCTGATGTGTTTGTGCGCTTTTCTCTATTTATAGGGCATCATAATTTCTCAAATATAAGG
TATAATATAATCAGGACAAGACTTGGGAACAACATAGTATACATAATAATCACATAGTTATTATTTTGAC
AAACATTTATGCCACACATGCAGAAACACAAGTCTGCAACCCTCCCTATGTATTGACATTATTCTGCAAG
TATGCTTACAAAACAATATAAGAGAGATCAGAATATCCTAGTATTCTTAAGGTTAAAACCGAAACCTTTC
TTGTATTTCCTGAATGAAATGACAGATTAATGTTATTGATGAAATGAATATGGCTTGGCTTCCTTGAGAC
ACAGGCTGTTAGTAAAAAAGCAATTAAGAAAATAATAAAGAAATGAAACAGCAATTCCTCTTCCTTTTTC
CTTTCTTCTGTACTGTGTCATAGCAAAAGTAATATAATAGCAACTAATTATAGGAAATCTCCCAGACTTT
GTAATTGGATCTTTCATTTTTTCCCACAGCAACTGTGAACTAAGAATTTATTCACAGAGCAGCTGTAGTT
TGTTTCCAGTAACTCTTAGCAGTGTCTCTTTGTGGAGTCTTAAAGAGACCCATGTACATATTTAGGCATA
TTGGCTAAACTAGTGCCTTTTAAAACATCTGCAGGTCTATTGTTGCCAATTCTTTCATTGTCTTCTATCA
AATTAACTTTTCTTAGACGGTATAAAGTGGGAGAAGCCACTGAACTCAAAGCTCAAAGCCTGACTTCTGG
TCATAGCTTCAATGTTTCCTTAAACATGACTTGGTAACAAGCACCAAACTTTGTAAATCTCAATGTCACC
TTTGGTAAGAAAAATGAAAGGAATCTTTGCAGTTTATATTTTAGAGCATTGCTATATGTGAAAGAGCTGT
GAAAATTATAAACCAGTGTAACTCACCATGTCATTATATAGATATGTCTAAGTTATTCTTGAATGTGAAA
TTTCCATTTGCTTGACAAATGTCACTCTATTACATACATGATAGTTAAAAGCATCTTATTTTCCATTTGT
TCCCCAGCCCAGTTTTCAACTTTTCTTCTAAAAAGGTAAGGTCAATTAAGAGATATTTCCAAAGGAAATA
GTTCTTTAGAAAATACTTTAGTATATCAATGCACATAATAATACAAAGAAATGCAGTTATAAAAATAAAA
TATAGAATAAATACACAGCTACCATATTCTTGGCATATATTTGTACATGAAGAAATAAACTTCAAAGCCA
GGCAGGACGGCTCATGCCTGCAATCCCAGCACTTTGGGAGGCTGAGGTGGGTGGATCACTTGAGCCCAGG
AGTTCAAGACCAGCCGGGGCAACATAGTAAGACCCCATTTCTATAAAAAATAGACAAAACTTAGCCGGGT
GTTGTGGCGCCTGCCTGCAGTCCTAGCTACTTGAGAGGCTGAGGTGGGAGGGTCACTTGAATCCCAGAGG
TGGAGGCTGCAGTGAGCTGTCAATGCACTACTGCACTACAGCCTGGGTGACACAGAAATACACTATCAAA
AAAGAAAGAAGAGAAAGGAAAGAAAGAAAGAAAAAGAAAGAAAGAAAGAAAGAAAGAAAGAAAGAAAGAA
AGAAAGAAAGAAAGAAAGAAAGAAAGGGAAAGAAAAGAAAGGAGAGAGGGAAGGAAGGAAAAGAAAGAAA
GAAAGAAAAAAAGAAAGAGAAAGAAAGAAAACGTTGTGATATGATTAGAGGACTAAGTCTTAACGATTAG
AAGCCTGATTTTTAAAAAATTCAAAAACCAAGAATCAGTAGTAGTAGTTGAGAAAGTAAAACTGAGTGAA
AAGTAGTTTTGCAATCAATGTGGAAACACTATTTGGTAAAATGCACTTGTACTTGACCTTCAAGTAATAG
ATATGTCAATGTTAAAATGGCATGTTTTGAAAGAAAACCCATGTCAAATAGTCGATATAAATTATAGATT
ATATTTCTCAGATAATTATAAATTATAAAATTTCTGAGTACATTCTTCCCAAGATGTGTTATTTGAGGCT
TTTGTCTTCACTCATTTTCATTAAGGAGACTGGTTTCCCATTTGTTTTCTCTTTTCTTTGTTTTCAAGCT
GTAATACTGTAGAGTAAACCCTAGCCAGGTGCTTGTCCTGCCTTACTTCATCCCCTCTTTCTAACAGACT
ACCCGTGTAACTTCCTCACCAGTGAAATAGGAGACATAGTTCCTGAGGTTAAATCATTTACTCAGGTTCA
TACAAGAGTGGTAAAGCCAACATTTGGACTTATAATTTCCTACAATATGCTACCTCTCTAGATAGTAGAA
TTAAATTTCAAAGAGAAATGTCAGGAGAAATTCCATATTCTACACTAAGGTTCTCATCTATTTTATTTTC
CACCATATTATTACATAGAAGTTATTTAATTTAAAAGTCTGATTTTAAGGTAACCTTTATACTATTAACT
CTCCTCCTTGAAGTGCTACATTTACAAGTAATTAACAAATACTGTTTGAGCAAGCACTTGTTCAGGTGCC
AGGGACTCCAGTTTTTGTTAATTTAAATATAGATGCTTGATATCATTTATTGGGGTGAAAGAGAATAAAA
CACCTTCTCACCCACCTTCCTAAATTCATTATAGTTAACAGAGGGAGTCTATAAAAATACTAGGACATAG
GGACTCATTACATTCTAACAAAATTCTAAGAACATGCATGTCTTTCCCCAGGCATTTAGCTTCCCCTCCC
TCCTCCTATTTTTTCATATCTTAAAGTAAGATCTAAATTAAAAATTTAGAAATTCCCAAATTATGTTGCT
GTTGATAAAAGGCCACTACATGTGAAATCAGCCTTTTCTGTGATACATTATATATCAGAGAGTCAGCTAT
TACTTGTTTGTGCCTTTAAGAAAGTTAAGACCAACTTACCTGTCATTTTAAATTTGATTTCCGAAGAGCA
GTGTGCCAGAAATTTGGCTTAAACAGACCAACAAAGCGTGGCTTCTAGGTGTTCCTGATGGGCAGGCTTT
TTTCAATTAACTTCCTGGGGATTGTTTTATTTAATTACAAGGACAGTTTACCAGATAAACTACTCACTGA
TTGAATTTCTAAACTCAAGTCTGCTGAATAAAGCCAAATGAGTCATCGATAAAAATTCAGATGCCAAGAC
CTTTGCAACAGGTACATACTCTAAGCCATGTATTTCTGCATTTTAAAGTTTTAAAACATCCTTCCTCTAA
TGTTATCTTTTTTTTCTATACTGGCCTGGGGAGTAACAGATCTTAGTTCAAATCCTGGCTCTGCTACACA
CAGCTGTGTTACCTTGAGCAAAGTATCTAATTCCATAGATAACGCTTTAGTTTCCTTAGTTGTGAATTGA
AAAATTCAGTGAACCCAAGTTGAGACGCATTCTACAAAATAACTGATTTGTATTCTGCCCAATTGTCAGG
TCACGAAAGAAAAGAAAACAATTGAAGTTCTGTTGCAGATAAAAAGAGACTAATGAGGTATAACAACTAA
ATGCAATAAAAAATGATGTAATTTAAGTAAGATTTGCAATTCAGATAGTAATATCATACTGGTGTTAATT
TTCTTGATTACTATGGTTATATAATTGTTAACATTATAGGAAGTGGGGCAGGGGGTACAAGGGAATATTT
AGTACTATTTTTGCTCCTTTCCTATAATTAGCTCACAATAAAAAATTTAAAAAACAAATAAAAGTTATGG
AGAGAGGAGAAGCCAACAAATAGCATAAGTATAGAACTGTTTTTGTACACATGGGCGTTAGACAGATAGT
AATAGTAATATCTTGTATAATTTTAATATAAAGAGCACCGAAAGAGAAAAAAAAATAAGCCCTAAATCAA
TGCCGAGGGTGTCATCTTTCTTTGGAACCCCCTTAATGCAGTGACTCCTGTAAGTGGGCATGTCCCTTTC
ATACAGAGTTGCAGTTGTTACAATTGATAAGGGTTCATGTGAGACAAGCAATTTTGAAGCAGGGCAACTC
CCACAACTGCTCAATGTGGACCTCATCTCCTTGTATTTGCATAGATATGCTTTGCTGCATGGATTGCTGC
AAAATTGCTGAAAAGACTCCCATACAAGGAGAATGCCTGGCACTACTCCTCCGTTTGAAAGCTCTGGTTT
CTCTCTTGTATCCTGGGAAGAGTGCTGCCAAGTAGTCCATAGACCTCTTCTTATTTCCTTAACTTGCTGT
GTACATTTTGAAGCATTTATGTGTCAGTTTATTTAACTCCCCCTGCCTAATAATGATAATAACAATAATA
GAGGTATGTATTGAGTTCTTATTGGTTCAAGTATGATTCTAGAGTGTTTATATGCATTAACTCATTTAAA
TATTCAAAATCCTTTTCATACTCATTTTACCGATGAGTAAACTGAGATACACAGTTAAGTAACTTAACTA
AGGTGACCTAGCTTTTCAGTGTTGGAGTCTGGATTAAAAAGAAGGTAGCCTGGCTTGCACTTTTCAGCCT
TTAGAAATATACATACGACCATATCCATCTGCTGAATTCCAACTCAACTTTCCTTCTTTCCTATACCAGC
TCAAACCAATTTCTAACCCCTTGTGTTTCCAAGGTACTTTTAAAATACTTCCATTCAAGCTGTTTTGCCA
TGTTTTGTACTATGATCATTTATGTACACGTCTTTCTTAAAAACCATTAGCCTCTTGGGACTAGGAATAT
TCCTGGGCAATGTCAAAATACTCTTTATATGCTGGTGTCTCCAAAGTTACTATTTCCAACCCAGCCTCCC
ATTTTAAGTTCCAGAGTAACATGGTTAGCTACTTATTGGACCTGTCTACTTTGTGTCTCCCAGGCATCTC
AAACACAGGATGTCCAAAATTGGATTTATCATTTTTCTTCCCTGTTCTTTAAGCCTCATCCTCCTAGTTT
CCTCTCATAAGAAATCACTGGAGTATTAACTTGGCAGGCCAAGTCAGATACCTGAAAGTAATCTTGGATG
CTCATCTCACTTCTGGAATCCCGAGTTCAACTGCCATTTCCAGCCTTTCATTATCTTTCAAATGTAACCA
CATACCTATACCTCAATATGTTTCCATACCCCCACCAATCATATCAGTTTTATTCACTTGGTAACCGATC
TTTCTGCTGTAAATTTACCCTCTAGACTATGTGAAAAGTGAGTTTTACAAAATATGGACTTGATCATTTT
CCTGCTTAATCTCTTCAAAATCATACATCTACCTTCCTCTGGACCCCCTTTCTCTCCACTTTCTTTGTTA
AGGAAATAGTCCCTTAAAAATAATGAAAATAATCAGTCAATATTTTCCAATGAGTTAGTGTCAATGCCCA
GGGTCTACAACTCAGCACAGGTATCCTTTATTAAGTGTACTATAAAAGGAGTACCAAAGCTTAGAGCTTA
ATCACTGGATGACACTGGAATGATTACAACTGAAAACAGTTAGTGATTAGCCTTGGTGGATATATTGGAA
GCCTAGAATACCTCTCAGAAGATATAGAACTAAAAACCCAGCCAGCCTCTCAGGCCGTTTACAGTGTAAA
CAAAGCAATTAGAGCCAAAGGAGCAAGTAAGTTCTAAGGACCCTGGCATTGTTAAATGGAAAAAGCCTCC
AGGTGAAATTGTGGTCTAAGCATAGGTTTCTCAACCTCAGCTTAATCGATTTTGGGGGCATTCCTATAGG
AACTTTAGCAGCCTCTCTGTCCTCTACCCACCAGGTGTCCATAGCAAGCACCCTTCTTCCTCCATGTTCC
CATGAGGGAGGAGAGTCCGATATCTATACAGTATATGAAACTAGGAAATGTATGTAATGATTTTGTCCTG
CACCCTTTATTGGCTTTGTCAAGACAGCCTAAATTTCCTGTATCTTAGCTTTTTTCAATAAATTACAAAA
ATTTGACAGTTGGAGCCATTTTTCTGCGACAGTAATTGGTGATTAAATCGCTTAAATGCCAACATTAGCC
AAAATGTTTACCTCTACAAATGAGAATCTTTACAATCCCTATCCTCTTTCTAGCAGGTTTGGCTAAGGGA
ACACCAAAAGCACAGGCAGAAAATTACAATTACAATTGTCCCTCAGTATCAGTGAGGGATTGGTTCTAGG
ACCTCATGAGATACTGAAATCTGGAGATGCTCAAGTTCTTTACATAAAATGGTGTAACATTTGCACATAA
TCTATTCACATCCTACCGTATACTTTAAATCATCTCTAGATTACTTATAATACCTAATCCAATGTAATTG
TTGTTATTCTGCATTCATTAGGAAATAATGACAAGAAAAAAAACTCTGTACATGCTCAGACTGTGCTTAT
TTTTCATATTCAAGAAAGGAAAAGAATGGTCTTTAAGCCCAAGTTTCAAGTTGTGAACAGGTAATCATCA
TTATTTATCATTAACTCAGAATCAGTACACAGGCTCTGAAAATCAGCTAAATCTGTGAAATTCTCATTCC
AGTGGCCATGTTTCTGAAAATAAACCAGTCAATAACTGCCTCACTCCAGTAACTATGCTTCTGAAAGTTA
GCAAGTGAAGGCCAGTCCAAGCTTTTGAAAGTCTGCTAATGATAAACTCCCAGTCCCGAACATCACCCTG
TCAGTGCCTGCCCCTATGGTGCTAGAGAACAGCCAATCAGTAAGTGTCCCACTCTAGTGACCATGTTTCC
AAAATTTAGAAGGTCAACGGCAGTCTTACTCCAGTGTCCACACTAACACAGCTGGAAGTCCTTGAGTCTC
TAAACACTCTGCACATTTCCCCAAACCTTATATAATGTCAGCTGTGGCTTTGTTCCAGGAACTTATCTCT
CACAAATACAGTTCTCTCTTGCTGGCAAACAGAACGTTTAGATTTTTGGCATCCAATATTTAGTTCAATT
TACTGTAAGTTCACAAGTTTGCAATCCTTTCATTGGCTCAACTCTTGTTTATGTTTAGTTATGAGTTTGT
ACCTGTGAGTGACTGACAGTGACAAAGCTGTGAGATTATCAAATTCAATCTTTTCAATTCCTGGGTCAAT
TACTAATATCCATTTTTTATTTGTGTAGAAGTAAACTCAAAAATGTCAAAATGCAAGTAAGTTTTGTGAT
AAATTTTATGAACAATGGATGTGCATTAAACAAAGAAAAACTCCTTTTGAAGCTCTTTGTGCAGCATACA
ATTGTTTAGCTAATATTGAAAATGGTGAAAACTTGGATGTAAGCCAGCAAATACTAACAGCTAAGCAAAT
AGATGTATGATTTCCTCAACTTGTACCAAAGAAAGTAATAAAATCACATCTTATTGAAAGAAATTCAGAT
GAAGAAGATAAAATAATTGCAGCACAAGTTGTCTTCCACTTTGTATATCATACAAGGTTTTAAGCTCACA
TGACTTAATTTGAATTGTGCTACAAGTATTTTTTAATGCAAAATTGTAAGTAAATTTTCTAGTGCTATAG
GACAAAATCTACTGCCGTAATAAAAATTAGAATAACTCCATTTACTGTTACAGAGATTACCACAGCTCAA
ATGTTTTACTTTTTTATGGCATAGCCATGAATGCAAGTAATCACAATTTGGGGGAAAAATTCCTTTACTC
GTATAATAATTTTCTCATAAAAAGACTTGCTTACAAGGCTATTGTAACACGCCTTCCAGATGAGTCTTCA
GAAACAACAAGAAATTGTTGCAGAGACCCTATGATTGAGTTAGTAGTCATAAAAATTATACTTATTTTGT
GGAGGTAATACAAATATAAATTGGTGGATATTTGCATAAAGGTGTGATATTTATTGAAAGTTAAAGCAAA
GTATAAATGGGACTATGAACAGTGTTGCCTCTTCTGCCATATTCTACATGACGCTGCTCAAACAGCATCT
GATATCAGTAAAGTAATTGCCAGTAAAGAAATTGTCAAGAAATTATTCTATTTCAGCATTTATTCTGTTC
AGACTCTACTAATGAAGAATTTTTGTAATTTTGTTGGCATTCGTTATTTTCAGTTCTTTCACATTTTAAA
ATTCACTGGCTCTCTTTAATAGATGCAGTTGAGAGAATTCTTAGTTTATTTAAAGCACCGAAATCATACT
TTAATTCTGAAGAAAAGGTCTCACAAATTCTTGCTGACTTTTAAAATAACCCATTGAGTAAAGACTACTT
ATTTCTTGCTCATAGCATTCCATATCTTTTAGCAATAGGATTATGCAATTTGAAAGAAGAGTAAACATTG
TTAATTGATGCCCTCCATTGCTTACAAAGAGTTGTTGAATGTATTAAAGATAAAATGGATGAAAAGTTTA
TCCCTTGTATGCCAAGGCTGTTTTAAGAGGAACAACACAACTTAGGGACAGGGAAAAAGTCCGTATTGAA
GTGGATAATTATTTTTACATTTGTCATGAGTATATTTTGAAATGTATTTTATCCCTTGAAGAATTTGATC
CTTTAATGGAACAGGAGAAATTATATGCACTCAAAGAAGGGAATAAAAATTCATGACAATGTTCTGTTTT
GTCAGTGGAAAATATTTTTAAAAGTTGTAGATCAAAATATGCAATCTAATCCTGAAAAATAAAAAAAAAC
AAGTTATGTCATGAAATATGGTATTGTGTTCTTTAGCAAAAGAGTAAGTCCCAGAGTTAACTTTAAGTCA
ATACATGTTCAGAAGTCCAGCTTATAAGGCTAACACTGAACATGTTCAATAAGCTGGACTTATAAGCTGG
ACTTCACGATGTTCTCATTAATGAATGTTGAATGGAGAAAAGAGAAAATTTGGATGTGACCACTATGGAA
GACATGTTACATTGTAGGGGGAACATAGGCTTTAACTATAAGGAATTTTATAAGTCAACTCTGCAAAACA
AGGAGCTGTTAATGAAAGCCAATCATCGAGGAAATTTTCAGAAAAGAAATCTCTAATTTACATACTGATC
ATTTATTTTAGATAAACAATTAGTAAATTTTAAGATTAAGACTGTTCTGTAAAAACATTTCCATTTATGG
TCAGAATATGTATACACAATGATAAATAATTCAAATTTAACTGACTGTGTATTCTTTTTGCTAACTCTGG
TAACTCCACCTGGAGGTAGGGAAGAATTATCCTGTTGAGGACCACTGGTTTAGACCAAGATAAAATATTA
TAGCATTCTTACAATTATTATTTAATAACTTTATTATACTATTTATATTTCTTAAAATAAATATATTCTT
GGATTTGCCATATTTTCTTGAGGACAAAAATATTTTTGAAATATAATAAAATATAATTTTAAAAGAAAAT
TTAATTTTAAAATATTTTAGAGTATTGAAAGTTTGTTTTATTGTAGTAAGTTAGCATCATCAGGTTGCTT
ATGGAAGGCTTATGGTCCTGCAAGATTAAATAATGGCTTGGTTTTGTTATATTTACATCTGTGATTTCAT
TTTTAAAAACTAAGAATAATAGTGTAGAAGATATAAAAGCAGTGTGTATATTTAAAGTGAATTTTAAGGA
CGGACTTAGTCCAAAATAAAAGTTTTGGACTGTGGATATTGCAGAAATATTATTAATAAAGATAATGAGC
AAAATCATCATATCTATTTTATAGTATAAGAAGTAATATGATAGGAACAATATTGAAATGGATTCTGCAT
TTACAGACTGCATTATGTTTCTTTTAAATCACTACTTCTGAGGTCAAGTGATTAATTTTCAAGTGATTCA
GAAGAAATGCTTTAATCACTTAGCAAAACTGTCATAGCCAAAAGGAGCTTGTTTTGGTCATAACCTAGTT
CCCACAAAGTGAGCCAAAAGTAATTTCTCATATTAAGCAAAGATATTGATTACAAGCAAGAGAAACAAAC
ACTGGCTGTCCCAAGCCAAATGGAGATTTATTGGAAGTTGTTGGGTACTCATGCATTCATTGGAAGGCTT
TGGGAAACTAGAGCAGCTGTATAGGAAAATGAGCTGACAAAAGGGAAATCTAGAGGCTGTTTGGCAGACC
CAGCTGGCTAGACTACTGGTCACTGCTGCCACACAGGATTCTACTATAGCTGTACATAGACCCAGCACAA
GGGAATTTCAATAAAATCTTCCTTTATTTGGCTTCATTCACCAAGTTCAAAGTAAAGGGCAACAGAATCC
ATTTGAATAAGCTTAGGAGATGATGGCCTCACCACTTAAAATAACAGGAGTGGAATGGAAATATAGTCAC
TTTAGTCACTTTAGCTTTCCACGGCAGGTGTCAAGGCTCTGAATTCCATCACCACTACACACAATGGGAA
ATACCTGTTCATTCTAGATAAGAAGGAAGATTGGATATTGGATAGAAAAAAAAGTAGACATATAATTAGG
TCATATCATATGACATTCTTTGTCATTTTTGACTTTTTATATTAAAGTAGTGATGGAAAGTTAGTGGAAT
ATGAAACAAAAGTTACAAGGATGAGCAATAAAATAAACAGAAATTATATTAACATTGAGCAATGAACTAT
AACAAAGTTTTGTCATACCAGAAGCAAAAGTAAAAAGTATTATTTCTCAAAGAAGAAGACACTCATCTCT
TATAGCACTATTAAATAAATTCTTGGATTGTGTAATAATCCAGTTTATAAAGAAGAGTGAATCATATGAA
TATGTTGAGGAAAAATATTAGGATGTCTTGCTCATTTGCCATAAAATGGCCAAACATGTTGAGATAACAA
ACTCTTCATACATCACTACATTGAGGATATTGATAACAGAGTGGTTGGGTGTTGTCGATTTAGAGTATAT
AAGATTAGCTTCTGAATAAGGACACTGTACAGGCTCTGATATCTTACAACTCATATGTGAAAGAAACTTG
ATGGAGGTGTTCCCAAATTTGACAGTAATACTGAAAATTTTCATGCCATTACCAAAAACAAGTTGTAAAA
CTCAGAGAAATTTGCTCAACTATCAGTAATGAACATACACACACTATACTCAGTTGTGGTAAAAGAAAGA
CTAAATTTTTCTATCTTCTCCATTACAAAATTGATACAAAGTAAAGGGAAGATCAAAGAGAATGAAGCCA
AAAATGAAGGGAAGTCCTAAAGAAATGTGTTAAGGAGTAAATTAATAAAATTTTGGAATTTATGGTATTG
GTTAATTATTTTTAATACAGAGACAACTTATTTAATTTTTGTTCTCACTTTTAATAAACATTCACTTTTA
TATTAATCTGTCTTTTGTGCCTAATTTGCCCTTGTATTTTTGCATTTTTTCTCAACCTCCTCCCCCAGTT
GTATAAGCCACAGATCCATAAAACCTGGATCTGTTTATAGGCCACAGTGGGTTAGATCAGGGGTTTCCAT
CCCCTGGGCCAGTGACAGATACTGGTCCATGGCCTGTTAGGAACCAGGCTGCACAGCAGGAGGTGAGCAG
CAGCCCAGTGAACATTACCACCTGAGTTCTTCCTCCTGTTAGATCAGCCTTGGCATTAGACTCTCAGAGA
AGTGCAAGCTTTATTGTGAACTGCACATACGAGGGATCTAAGTTGCACACTCCTTATGAGACTCTAACTA
ATGCCTGATCATCTGAAGTGGAATAGTTTCGTCCCAAAACCTACCTCCTTCCCTGCTGCCCTGAAAAAAT
TGTCTTCCATGAAACCAGTCCTTGGTTCCAAGAAGGTTGGGGACTGCTGGATTGCCAGGTGTTATCACTA
AGAGAGCAAATAGGTAAAATGTCTGGTAATATACACTGGAATATTATGCAGTAGTTAGAAGTGCAGATTA
GATGTATTTACAACAACATGAATGCTAAGTACTTTAAATCATAGTGCTTAGTAAATAAAAACAAACAATG
AGAAAATGTATGTAAATTAGAGATTCATGTGTACAATATTCAATTTTCAAGAAAACAGAAAGATGTTCAT
AAACACATTATAATGGTTGCTAAGCATGGGAATGAAAAACAGGTAGAGTGGCAGAGAATGGGATATGGGG
GCAATGGAGAAAAAATATTAATAAAAGGAGGCTTTGCATGGCCAATGATAACAATTGGCCACAAACTGGG
AATTTAACCTGGAACTTGAGGTTAAAAAAATCTGTAGAATGCAGGAACTTTGGTTGATATTATCCACAAT
ACAAGTAAGTTATGATTTAACCTTTAGAAGGAAAATAAGCTTTGTGCATCCTTTAAAATTTTTAATTAGA
ATAGTTTGAAGACTAGACAAGTACAGGAATAGAAAATCTAGAGAGTTTATGAAGCTATATATTTCAAATT
CCATCGAAGGACAAAGCCAAAGATGATTTTTTTTCTTGTTTGGAAAAGTTGAGAGTAAGTAAGGACAATC
AGACTTGATGACAAAGCCAAAAATGAAGGAGACAGCTCCTGGATCATAGCAAACTGTTAACACAAGAGGG
AGGAATTGTAGGAGATACCTGTCATAGTACAGACCAGCAAATTTCTACTCATCTTCCTTCCCTGACCAAA
ATCTTTCTTCCTAAGTCCAAGTTAGGTGCCTTTTCTGTGTGTTCTCATTGCTCTCATATAGTAGGTGTGT
TGTAATTGTTTACCTGCTGGTTTTCCCCAACAGACTGTAAGCCCTAGAGGACAAGGATCAAGGCCACCTA
GTTAGTATTGTGTCCCAGGCACCTGGCACCTTAAAAATATTTGTTGCTCTTACCTCCAAACAACAACAAC
AACCCCCCCCCAACAATAACAAACAACAAAAATGGGTAACAACTAGAGATGGTAGATATGTTAATTTCCT
TTACTATGGTAACCTTTTTTTTACTATCTACATGTATCCAATAGCATCATGTTGTATACCTTAAATATAC
ACAATAAAGTGTATTTTAAAATATCTGTTGAAGAGTAAGTAAAATGAAATGAACCTATTAAAGGAATATC
AGTATAATATTTCACACATGTGACCATTATTTTCATAACTTGCTGCGGGGGTAAACCTTAAAATCAAGAC
TACCTTCATTTTGTTTTGGTAATTTGAAAAGTAAGGAACCGATAATATTTAGGCATTTTTTTACTTTAAT
CTTTTGTAGTTTAATAAAGAAAAATTTCCAAACTACATTCTGTTAATTGTGCTAGCAAGTTATTATGATG
GCATTTTAAAAAATTAATGCAAATAATAATGATGCAGATAATCAATTTAATTGATGGCTGGTCACTTAAG
ATCGCTTTCTAATTTTCAGTTATTAAAAGGTCTTTCAAAATAAGGCTACTTTTGTAAAAAAATAATTTTT
ATACTATATTGTACATTAAGGGAATTCATGGATGGTGTTTTCTACACAACAGAGAAAAAATTTTATGTAA
ACCTGAGAGCAATTACTTTACCAATTAAATCACTCAAATGTTGTAGACTAAAATAATTGAAAGTCAAAAT
AAGATTAAAGAAAATCAGTTTTCATATACAAATAATGGTAAGGGATTCAAGAAAATAAAAATTGGACTTA
ATGCATTTTAACCTATATAGTTTTGAAAACCATAAAACATAAACCTTATTGCTTTGTGTAGTCATTTTTC
ACCTTTGTAGAGTCCATTTTTCCTTACATGTAAACAGGCGCCTATTCTAATATTCTTCATTTCTTGCTGT
GTTTCCATTCTTTTTGTCTAAGATCTTTTCCTTCTGCCTAAAGTACTTCCTTTAGTGTATATTTTAGTAC
AATGATTAATTCTCTCAACTTGAGTTTTCAGAAAAAGAAGTTTTTAAAAACATTTTACGTTCATCTTTTT
AACTTTTCATTAAGTATAACATACTTAAATAAAAATGCATATATCATGTTCTCGCTTATAAGTGAGACAC
ATAGAGGGGAAAAACACACACTGGAACCTTTCAAATACAGTAGAGGGTGGGAGGAGGCAGAAGATCAGGA
AAAATAACCAATGAGTACTAGGTCTAATACCTGGGTGATGAAATAAGCTGTAAAACAAACCCCCATGACA
CAAGTTTACCTATGTAACAAACCTGGACTTGTACTCCTGAACTTAAAATAAAAGTTAAAAAAAAAATGCA
TATATCCTAAAAATATAGTTTGATGAATTCTTCACAAACGAAACAAACCATGTAACCAATACTGGGATGA
AAACACAGAATTCCTTCACTCTAGAATAATATTTTCACTCTCTCTAATGTTCCAGAGTGTCAGGGACTAC
AAAGGCTACAAAACCAAATGACAGGTCTGCTTTGTTCCAAGCAATGTTACAGGCTAAAGATACACTGCTT
GTAGGATTTCATACTCTTAAAAGTTCCCAGGTACATAAAATTCTACTAATTAGTTAAATGAAGCTGTGTA
TTCATGGACAATAGTTAAATTGTGACACAGATGAGTGTACCTAGCACCTTGTTAGAGAAAGCTCCTTAGT
ATCAATGAGATGTGAAGGACATCTAAGAGTTATCAGGGCTAGAGGTCTGGGGAGAGTTATCCTGGCTCAT
GCTAGTCTTTTAGTGAAATGGCCTGGACAAATTCATAGTGAAGATGGGCAGTCAATTGATAAGATTATAT
TATATTTGACTGTTTATTTCTAATAAGATTATAAAAAGATTATAAAGTTAAAAGCAAATTGAGGTGTTGT
AGTGAGGAAAGAGGATTGTATAGATTCATAATGGCTGGCTCAATTCCAGTGTTTTTACTCTACTTAAGTG
TATAGATTTGGTTAATTTTTAAAATTTCAGGTAACAAATAATATGGTCATCATTGAAATCCCTAAATATA
ATAAATTACAACCATTGTTTTTACTGAATCTAGACTAGGGGAAGAGTAAGATAAAACAAGATAAGTAGAA
TGTTGGCAATTTAAAAAATTGTGCCAGGCTCAGTGGTTGATGCCTGTAACCCCAGCACTTTGGGAGGCCA
AGGAGGGCAGATCACTTGAGCCCAGGAGTTCAAGACGAGCCTGGGCAACATGGTGAGACCTTGTCTCTAC
AAAAAATGCTTTAAAAAATTAGCCCAGCATGGTGGCACGCACCTGTGGTCCCAGCTACTTGGGAAGGTGG
GAGGTCGCTTGAGTGCACGAGGATGATGCTCCAGTGAGCCGTGATCACAGTATTGCACTGCAACCTGGGC
AACAGAGTGAGTGAGACCTTGTGTTTATAAGAAAATTTATACCTTTCAAAAATTTAAACTCATACTTTCT
GCCCATATTACAGCTGCCTCCTTCTGTGGTATAAACGCAATATTAAATATTGTGTTTATTTTTATCAAAA
TAAGACAGAAATTCTCAAGACAGAAAATGAAAAGTTTTGTCAAGGGGTTTCACAGACCTTTCATTGAATT
TTAATTGCAATGCTAAACAATATTTGAAGATAACCCATAGTGGTCCCAGTCTTCAGCCTGAAGTCAAGAA
CCTTTAGTTATCTGAATGTTCTCAAGCTCCAAGACTCCCTAGGATGTTATTTAAGCAAAGTGCAGGATTT
CCAAACACTAGTGATTAATATATGACATCTGCTTACTACAGCACTGTTGTGCAGCCACAAAATCTTTCAG
AAAAGTTCACAATAACTATGGCTTACTTTGATAAAGAATAGTCAGTAGGATGGTTAGTCTAATATGCTCT
CATAAAAAAAGAAAAGTATTATGAGGTGGTGCCAGGACCTTGGGTCCTATTTGGCTATTTCTCCAAGGAT
CTCTCAATGGTAAGATCATCATATATCTAAGCTTGCCTAGAACAGTCCAAGGTCATTCCTGCTGTCTTGG
TATTTTCTGTGTTGTGTAGCATTTATTCTTGACACAAAATCCTGGCTTGGAACATAAATGCTGTAGACAT
CCTGCCAAGGATTGAATCAAATTTATAAGTGTAACCCTGACAGCTAGGGTAGAGCTGTCTATTTCCTTCC
TCCAAAGATGTTATCAGGTATTTAGATTAGTAAAATGAGAAATACTGAGATACCCTTATAATTCAAGGAG
CACATAGGAATGGATATAGACTTGGTTTAAGTATTATTTTTCCCCTGTATTCTCCAGATTCAATCGTTTC
GCACCTTTGACTTTCTTATCTAAACTGTAGAGCTATTCAATGACTAAATTGTACTTCTTATTGTCTTTGA
ATGTTTCAGCTTTATCTTGTTATTGACATATAGAATTTTTATTATCTTGATATTTCACTTATTCAATCGG
AGGCCAGGTGTAGGAAGGACTCCAATATTTTCTTGGTGCCAAGACATAGAACAGAGTTTTCAGATCCATC
TGGAAATTTTTCTGTTTTCATGGGAGAAGGCCTATAAGAGTTCATACTGGAAGGAAACATTGCCTCTCTC
ATCTCTCCTGATGACATATTTCTCTTTTATCATGGGAAGTCTTATGTTATGAGTAGATAAGTTATGAAGG
TGATGTTATCGAACATAGGAGAGTAATTTAAAATATTATTGACAAAAGGGGCTTTTGTAGTTATAAACCA
GGGTTAAGCGTATCCTTGTCAGCCAGAGTGGAGCTGTATTTTTGTCTTGCTCTGATGATGTTATCAGGTG
TTCAGAGCTCAGTAAAATGAGAATCTCAGAGTCAAATACATTTCTTTTATCTCTATGTTACCTCACAGTT
CCTAAAGTAACACAGAAATCAAAAGTCAGCCTTTTGTACCTTAACGGCATCATTAAGATGGATATGTAAT
AAAGCTTCAATTTAACAACTTAAAAGATATTTAGTATGCTTACCATGGTTGAGGGAGTTAAATTGTACTT
ATTCTAAAAGAAAATACACACAGATTCCCTCATAAGTGTATTTTTTTAGCCTATTATAATAAGACATAAT
GACTCTCAAGTATAATTAGGACTCAGACATTGTGACTCCAGTTTAGTTCTATGTTTATAAGTCATAAGCT
CATTCCTGACCCAGGAGAGTGCATTTATAGAAAATTATCCTCCCAGGCATGATTACTTTAAGAAAACTGA
TCAGCATTTCTAATGGATCTCCTATTGATCAAAGCAATGCTATTTTGTACTGTGTAAAATATGTATTTTT
AATGTATTAATTGTAATTCATTTTAATTAAAAGTGGTTCAAATTTATGTCTATTATCACTTAGGTATATT
ATAGATAATATAGATAATGGCAATGCATTTACCTGATAAATTCAAATTCACTGAGCTATTATTACCTTTA
TTTAATATTAGAATAATTCTACATCTGTGATTTCCTTTTGCTTAAACTTTTGTCTTGGAATAGATTTATG
GAAAAATGGTGCAGATAGTAAGGACAGTTTCAATATACCCCACATCCAGTTTCCCCTGTTATTAACAGTT
TATATTAGTATGGCGCATTTGTCACAATTAACAAAACCATGTGATACATCATTATTATTAACAAATGCCC
TTACCCTATTCAGAGTTCCTTAGTTTTTCACTAATTTCCTTTTTCTCTTTCAAGATCTCACCCAGGCTAC
CACATTACATTTAACCATCATACCATCATATCACCTTAAGCATCTCTAGGCTGTCGCAATTTCTCAGGCT
TTCCTTGTTTTTGATGATGTTGATACTTTGAGTGAGTATAGGTTAGGTATTTTGTAGAATGTTCCTGAAT
TGGTATTTGTCTGATATTTTTCTCATGGTTAGATTAAGGCTATAAGTTTTGGGAAGGAAGATCACATATG
TAAAAGGCCATTCTCATCACATAGTATCAATGGTACACATTGTCAATGAGTTATCGTTGTTGATGTTAAC
TATGATTTAAATAAACTTTTTAGGTTCTCATGTTCTGATTCTATGGTGCCTTGAGAATGCTTTAAAAATT
AATGGTACTTCAGTCAAGATTTTACCTGCAGGAAAAAATCACTATGTGTTTCTAGAGAAGTGACATTTTC
AAAGCAGTATTTCATTTTACAACAGTTAGCATCAATAAAAATGTATTTTTTATTTATCTTTACATTTGTC
CACTGTCTTAAATATTTATACTGACTCAATCTCATTCAGATTTTGGCAGATCTTCCAAATGATTAATTGG
ACCAATTTTAAATAAATAAATTTATTTATTTATTTGAGATGGAGTCCCGCTCTCTGGTTCAGGCTGGAGT
GCAGTGGCGCGATCTCGGCTCACTGCAACCTCTGCCTGCTGGGTTCAAGTAATTCTCCTGCCTCAGCCTC
CTGAGTAGCTGAGATTGCAGGCGCCCACACCACACCTGGCTAATTGTTGTAATTTTATCGAAATAAGATT
AAGTTATTTACAGGTAAGAGGCTCAGTGATGGAATGCAAAACAGCCTGAAATTATTATAGAGTTGTGAGC
ATGTAGCTATTTTCCTGCTCCTTGAAATAAGAAAATATAGAGTTGTGAGCGTGTAGCTATTTTCCTGCTC
CTTGAAATAAGAAAATGTCATATCATAGTCCCTCTAATCAAGATTAAAGAAACAAACAAGTAAACACTAC
TTGCTTTCTACAAGAGATGTCAAAAGTTATCAAAGGAAACATACTGTTTAAAGGAATTATCTGGGAGATA
TTTTTAAGAGAATAACTTATTCTCTTAAATAAATATAGCTTTCATATATAAGATAAACATTAATTATTAC
AAAAGCACAACATATTACAAAATTATATGGACATAATATGCACATACTCTAACTGAGCCACAATAGAATT
GGTGCCCTGACACTAATAAATCATTTGGGAACTGCAGTTTGGAGTATTTAGTACCTGTTATGTGGCCAAG
AAGGTAATAGAGGCCACTGAGACCTTTTCCAGTCTAATATAATGGGCACAACTCACATTTTTTCCTTTTA
GTTATTAGGTAAAAATTAGTGTTGGTGTTTACTTCAGTGATTTTTTAGAGGATTTACAATTTTTACTGTG
AACACTTAGGGAAACCCACTTCCCACCTCATCCTATAGATTTTCCCTATGTGAAGGGTTTTCATAGTACC
TTGGGGATGAAAGGAAGTCCCCCAAATAATTTTCCTGATGTCTGCTTCCAATATTTGACTTCAAGAATAC
CGTTTTCCAAATAGTATGTTCAGAGCCATACCAGGCACACAGAGTCTCTAAAACTCTGGAGAGACTGTAA
TATTTCTTTGCTTGAAGGAAGGGATCATTCTTGTATTCTGTTCAGAGATTACATTAATGATATTTAAATG
TTTCTCAGAAGATAAACCAAGTGTTGGAATTTATTTGCTTTTGACCCTGTTCAGCAATATTCAATGAATA
AAAAAGAAAAAAGTCTTAATGATAGTTCTTGAGCCACATGACTTTGTCTAATTAAAAAATATTTTGCAAG
TCTTCATATAAAGTTCAAAACAACAAAGGTGAAATGATGGGAATTTTCTTAAGAAAGGAAATTCTGCAGT
GACCCAATCCTTTGAGCAATCAACAGTCATTCTAGGGATACTCTTGTATTGTTTAAAATTACATTTTACA
AAATAAATAATGTCCTAAATGCCTCAACATTTTTAGAATCAGAAAAGTTGCTTAACTGGGATGTCCAAGA
TCATTTAGCTAGTAAGTAGTGAATCCAGTATTCAAAACCCAGGCTTTCTAGCTCCTGAACTTGTCCTAGA
CTCACTACTCTAAACTAAATCTCTGTTTAATATTACATGCTCATATGAAATGTTTCATGTTTAAAAGGCC
AATACATTCCCATAAATGGAAATATCCTCATTTAATATAATTAATATATTTTAATTATGGAAGCATGATA
AAATTATCCAAAGCAAGGAATTAAAATAGAAGGATGAGACAAGTTAATAATCTTATGAGCTCCATGAATA
CGTGTTCATTATGTGCAAAACTCTTCCTGTTAAGCACTTTATATACAGTTAACCATCACAATCTAAACCA
CTCTTCGGAGCATGATTATTAACTCAATTTTTCAAACTAGGAATCTTAGGCTCAGAGAGGTTATGTCATC
TCCACAGGGTCAACAGCTCTAAAATGTGGAGCACAAATTGTAATGGGAAAGGCCAAGATGATTATTGACC
TGTGAGCCACTGAAACCAGTGTGAATTGTTCTGTATGGAATAGAAGTACACTTCATGATATATATTGTAA
CTGTTGCTGAAGGATTTTATTCATTTTACATTGGATCAAATGGAAATCGAGCTGTATATTAAAGATTTTA
CAAAAGCTCCAAATTAAGAAGAAACTATAGAAACTCAAATAAAATGAAATTTTTTTTAAAAAAGTGTTTT
CTAAGTAGTGTTTCATAAAGTAATACAAATTTTTAGTTATTAGGCTGATAAAATAAATCTATTTCTTTTA
GTATCTTTGTCCTTTCTTTTTGAATGTTGTTACACATTCCTGAAAGTTGGAAGATCAGCGTTTTAAAATA
AATGCTAGGAAAGACCTAATGTAACTCTTGATTATTTTACAGCATCAAGAGTTCTTCCTTCTTCTTGCTT
ATTGCTTGACAACAGATTGACTGAGGATGCTTTAGAGCTGACAGGTTTGGAGGATGAGGGATGAGTGAGT
ATGAGCAAAACTCTGAGGAGGAGCACTGCATGCCTCAAAAGAACTTGGAGAGAATAAGAGTTACCAAAAA
AAAAAAAAAAAAAAGATTTTGCCAGGCTATAATTGGAATGAAGTGATATAAGTGACATGAATGATGCTTA
AAATGTTGAGAATTAAAAAGTAAATATTAAAACATTGAAAAACTTTTTTTTACCCTTTTTGGTTTATCAT
CTTCCTTCTCTCCTTCAACTTCCATTTTTTTCATTCAGTTTTTCCCCTCATTCCAACTCTTCTTTCTTTA
ATTGAACATAATAGAGACTCGGAGTTCAAAATATGTGTTGTTTCTATTGAATTGATAGATTTTTTTTTTT
TTTTTGAGACAGGGTCTCTGTCTGTCATCCAAGCTGGACCGCAGTGGTGTGATCATGGCTCACTGCAGAC
TCAACCTCCCCAGGCTCAGGTGATCCTCCCACCTTAGCTACTCTCAAGCAGCTGAGACTACAGGTGCACA
GCTCCATGCCTGGCTAATTTTTGTACTTTTCCTAGAGACAGGGTGTCACTGTGTTGCCCATGCTGTTCTT
GAACTCCTGCCCTCAAGTGATTCACCCGCCTTGGCCTCCGAAAATGTTGGAATTACAGGCACGAGCCACT
GTGCCTGGTCTATTGAATGGAATTTATGTTTATCCTTTCCAATTTTAAAATAGTTAACTTTAAAGTTGTG
TATTTTACATTCATTGTCTTCAAACCAAACAAAATCCAAAAACTTTTCTACTTTTTGAATTGAATTTTTT
CTCTCCTTTTGAATAATAAAACCTGGTCATTGATGTATGTGTACTCATCTGATATTTTTGATTTAACAAA
ATCAAATGGAGGGTTCACGTTAGTATTTTATGCTTTTTAAAAGTAGACATGCTTATGCTCTTCCCTAAAA
CTTTTATTTCAGCATTACTTAGATAAGCCCTGGCATCTGTATTTTTTTAGGTTACACAGATGATTTTTTT
GTATACCCTGGTCAATAGCCACAGTGGAGTATACCTGCATTCTGATATATACACTCTTGACAACCATAGA
GACAAATGATACTTAGCTGCATTTTTAACATTACCTAACATAAGTGAAAGGGATTTTAAAACAGTAGTAT
TTTGGAAAACAGCAGATAATAAAACATTAATTTTGTGTTGTGGGTTATTGGTATTAAGTATCATGTAATG
CCCACATCTCAAAGCTCACTATGGAAGAATTGGTGTCAGATAATAGGAAGATTGATATTGTTGAAAACCA
TGCATTCAGCAAGCCAGCCATTGGTAGCACCCAATGAAGTGTTCAGTATTCATCTAAGCAGAGAAAGTAG
AAGACACAATCTTTACTGTAACTTTGAGTGTTTGCAGTGTGTCAGATGGTCCCTTGAAGAAGAAATATTA
ATAAAAATGAATTGCAAATTGTCTCCAATTTACCTGAAAGAGATCAAAATAATGTATCTAAATATATGGT
GTTTGAAAGGTGTAGAGTCAGTCCATATTTTTCAAGCCCAGTGAAGGTAACAGAACAGTTTCTACTTAAG
CAGCTGTTTGTTGGTTTCTTTGGCTCAAGAGAGAGGTATAATATCTTCTTTTATTTTACAGCTTTTTAAT
CATAATAATAACTATTATAAAATTAACAAATGCACATGGTAACTAATTCAAACAATTTTTGCTTTATCCC
TTTAAAGCCATTATCTAGGGAATAATCACTTCCCGCTTTTTTAAAAAAAAGTTTTTCTGATATTTTCCTT
TTTATCTCTAAATAATATGCTTATGTCTCATTTTTATTTCATCAACTTATGTAATGTTTTCTAACTTTCT
AATTTGAAAATGAGGAATTTATTTTATTTATGTAAACTTTTCTCTTTCTGCCCCATTCTCAAATTCCAAG
CAGTTATGTTTTCATTTCTAAGTAATTCTATGGGTTGGATTTATACCTTTAAATGGAATATACCTGAACC
TCTATTTCTTGTTACTTAAACTTTAGCCAGGATGCACTAACTGCCTTCAATGTAAGATAAGGGGTCAGCT
CTCCAACTTTTCCCTCACCACTAATCCCCCTCTACCTCTCAATTTTTGTAAGCTATTCCATTACTTTTAC
ACTGATAATATTTAACACATATATGTGTATATCTTGTCTTTGTGTAAATAAAGTTCTATAATTGTACTTA
CATTTTATATATGCTTTGGTAATTGGCACATTTGTGAGATGATTAACTGATACAATTTAACAATATTATA
ATTATGTATGTATTTTTCATTTTAAAACAAGTAACATGGTTTGATCTAGAGGAGGAATTTAAACCTCTCA
TTCAAACTCTGCTACTTGAAGAACACATCAAACTCAAGTGGATCCTCTCTGCTTCAATTCTACCAGTTGT
TCAAGTTCATGCCTCATTTTGAATTGATTCCTATTTGGACAATATGGCTATTTACACGTTCTAAAGAAAC
AAATTCCCTTTGCTTTTATTCACACTATTTGTGAAACATCTTGTATTCTATGTGTTTTAGAAGTACTTTT
TTTTTTTTGCTCTACCTTAATCCAAAGTAACTTAAAAAAAAAAGATAAAAAGGAGTTAAATTTTGAATTT
TCTTATACCTAGAAATATATTTTGTTTGCCATCACACTTGATAATAGGTTGTCTGATAATAGAATTTGAT
TCAAAATATTTTCTCTCATCACACTGAAAGCATTGATTGTGTTCTAGTGTCAATTATGGCTAAGGAAATT
TTAATGGCAATCTAATTTTCATTCTTTTTAGTTAGTTATCTTATTTTTGGAAGATTATAAAATCTTGTTT
CTATTGTTAATGCTCTAAACTTTCACAAGAATGGAACTTTTAAATTGATCTTCTTTTGTACTTTTAGCTT
GATGACTCTTGGTGAATTTTCTGCCATTACTTTGTTTATTATTTCCTATCTACTATTTGCTGCTCTCTTG
AGACTAATGTCGAACAACATGGATTATTCTGTATCTTCTAAGTTTTATTTTGTACTTTTTTAATTTTTAA
AATTCTCCTAAAATATATAGAAAAATCTAAGATATAGAACTCCATGCATTTTTACATATACATATTTTCC
CATGTAACCATCACATAGATCAAGGTATAGAACGTTTCTAGCACCTGAGAATGCTCACTGGTGCCCCATT
CTGGTAAAGTGCCCCTTTTGGGTAAAGTAAAAGGTAACTACAATTATGACTTCTCCTTTTCAGCAAGCAT
TCTACTGAAATTTTTTGTTTCACAAAGCATGACCTTTACCCAGAATTGATGTTGTTGTTTTTGGTAGCCT
ATTTTTTTTTTTTAATAAAAGATACTCTGTCTCCTCAAATTTCTCTAGGAATACTAACTTTAACTTCTCA
ATATCCCACAATGTTTGCTTTTTTTTTTCTAAAGTTAAAAGTTTGGTCTGTTCATCTTAGTCTTTCAATT
TTATGTTTGGTAGATCTTCATTACCAATTTATTCTTGATAATCTATTAATATTTATGGAAGAAAGGCCAG
GTTGGTGTTGGCCAGGTTTGCATCTGTCTTTATACAGGTCTGTTTCCCAGACAAATCCTCTCTTGAAAGA
GAGGGATGACTGAAAGGTCTGTGTATATACATGTATGGTGTACTGACAGGCAGGCTACATTTTAGGATGT
ATGGATGAGGAGTAGATAAGGGAGTCACCTTTCCCTCGACTCCACCCTGGTTCTAGCTAAAATGGACATG
GATACCCTAGGAGTCCTACCTTAATTAATGCAGATATTATCCTTTTGGTGATAGCTTGGAGATAAACCAC
TCTGCTACATATTTAACATAGAGAGGGAAGTGGGACAATGTTTTCTGCAACATTATTGAATAAATAATCC
ATGGATTAATCAACTTGTTTTCTGATCTACATCACATATTTGCCATAGAAATCTGTGACTTCTCTGCTAG
AAAGAAATTCTAACATTCTCAGATGAGTGTTCTAGACTGTAGCTTCTGCTTTGATCTCCTCACTAGGGTA
ATCTTATGATTTATGATAACTTTTTTTGTCTTCCTCTGGTTTTTAGTGATATTATCTAACATTTCGCCAC
TGTACATCTTTGTTTTTACTGCAATGGAAATTTGAAGGCCAGAGAGTGTGATAAACACATGGGATAAGAC
CATTTTCATGAACTGGGAGCTGACATTGCTTTGACTGGAAAGATGTGTGAAGAAAAATAGATGCTAGATA
GACAAATACATGATATGATATTTGGACAGACATAGATCCATAGATGGATAAGCATAGATAAAACATATCT
ATATATATGTGTGTGTCTGCATACATAGAAAATAAGAAAGAGTTCAGAATAAATATTTCCATATTGATTT
TAACTATAGGAAATCTTAGATACAAGAATAACACATTCATTTTTTAATTTTAGTAGTTGCCTAAGTATTC
TCTAATATGAGATTAATGGTGAGTGGGTAAGATAGTAATGAGAAAACAAAAGTACTGCCTTTATATGCAA
CATTGGGATTTCATTCTAAAGAAGATACAATCATAGTAATGTTAATAAAAGTGGAAATGTAGCAATATAT
TTTTCAAAATTGTAAATTAAACATGACAAGAATAAATATTGATTGATTTCATTGACTATATAAAATTAGG
TCAAATAATTTTAAATGATTTTTTTCAAAGTATATTTTCTTTCTTCTGGATAGAAAAACATGACATTTTC
GTTGTACAAATTTGAAAAGTAAAGTATAAAGACATAAAAATAATCCATAATCTCTTAACTAGGGCTAAAC
TTTGTTAAATGCATTGTGTATTTAAATTAAATATCATTTTAAAAATTATTATTAATTTACATCTATCTGT
GTGTATAACCTGGATTCCACTTATTATGCATTATATTTTTATAATTCATTTAATTCATTAAATAGTCTTT
GAACGCTCTCTTTCAAAGCTGCTCATTATTTCATTATACATTCATTACTTCATTTAGTGTAACCCTTACT
GTTAGGATTTTGGATTGATTATAATTTTTCAATATTATAAAAATATTGTAGTAAATATATTTATACATGT
TAGAGGACACCTGAGATTACTGTCTTTGAATAAATTCCTAGAAGGAAAATTATTTGGCAATATAAACGAA
TTCACGTGTTTAAGGCTTTTGATAGATATTACAAAATTACCCTCAAAAAGTTTGTACACTTTATTTATTT
ATTTGAGACGGAGTCTCGCTCTATTGCCCAGGCTGGAGTGCAGTGGCACGATCTTGGCTCACTGCAAGCT
CCGCCTCCTGGGTTCACGCCATTCTCTGCCTCAGCCTCCCGAGCAGCTGGGACTATAGGCGCCTGCCAAC
ATGCCTGGCTAATTTTTTTGTACTTTTAGTAGAGACAGGGTTTCACAGTGTTCGCCAGGTTGGTCTCGAT
CTCCTGACCTCATGATCTGCCTGCCTGGGCCTCCCAAAGTTCTGGGATTATAGGCGTGAGCCACCGCACC
CAGACAGTACACATTATTTATTACTTTCATCCAGACAGGCAACACTGATTTCCCCTATAACCTCTCTAAT
ATCACTATTACTTTTGTTAAAATCTTTGCTAATGATTAATAGGCCAAATTTGGTTCCCCATGTTTATTTT
AATATTTGTTTCTTTGATTACTAGTGAGGTTTGACATGGTTACAAGTGTTTATTAACCATTTGTATTTTT
AGATAATTATGTTTTTTACTAAATTTTATGAGATATATTAATATCTTTATTCATTAAAGGAAAAGCACTT
CATCTGTCATTTGTATTGTACTTTTTTTATTTTGTGGATGCCTTCCACTTTTAAAAACCTTCATTGTTAT
GAGATTAAATCTAGCATTCTTTATCTTTTTCGATTTTGCCTTTGGTGTAATGCTTTACAAGTCCTCTCTA
CTCTCACATTAAATAAGTATTCATTTTCACTTTTATTAGAATGTTTGAGCTTTATTTTTCCATTTAAATA
TTTACTCCACCAGAAATCTCAAATCTTAAACTAAATGCCTGACTAATGAAAACTCCTGTGGCCTGTTTAT
CCAGAAGACTAAGGGAAGAGGAAAGAAGTTGTAGTCACACATAGGTCCTGCCCCATCATTGCATCTTCTT
TGTGTGTGTTGGTGAAGTGTGAGGCTCCATTTTTACTTATGTTGCCCTCTGTAACCCTGTCAGCCTATCA
TCTTGCCACCCCACTGGCCTGGTACAATAACTTCCAAGCAAACTCAGATGCCAACTAAAGCTGCTCTTCT
TCAAAGCTATAAAATTAGTAAAATGAAAGGAAGTTTCACCTCTCCAAACTAGAGGATGATGATGAAATCT
GATCAAGGTCTCTTCAACTTCCATTTCACAGATCATGATGTTAAGCAGGTGCTTAGTGTCTTCCTTTAAT
TTAGCTTGATCAGGGAAATATTGCCCATGCATGGTACCACTAGCATTGACCACCCTTTGCCTCTGCTTTT
TTCATATACTTTAGTCATTCTGGAGCTGTGTTGAGCATGTGCATGGGGGTTGGGGGAGACAGAGACAGAG
GAGAATAGGAATATTGGAAGAATCAAACTGGTAATGGACATCTGAGTTCACAACATCTTTGTACCTAGAT
TTCTGTCAAATAATTCTTGAAAGAGATGGTACCCTCCAGTTGAATGTAGAATCTACAAAAACACCAGTTA
TTCAAGCCAACTTTTCTTCAAGAGCCACAGTATGTCTACACTGAAAGTCTAGTGAGGCAAGTTATCCCGT
CTTCTCACCTTTATGATTTCCAAAAGTGTTGAGGAATCTCCAAAGCACAAGTGGCAACTGCTTTGATTTG
ATTCATTGATTTGTTTCCTTTTTACTAAATCTCTTCCTATTCTGAGGACAATTTTCACATATTTTAGGAT
TCAAAATTCTTAATGCATATTCATAGTAGTTTTATTAAGATATAATTTACACACCATAAAAAATCACCAT
TTTAAAGTGTATCACTCAGTGGTTTTTAGTATGTTCACAAAGGTATGCAACAATCATCATTATGTAGTTT
TAGAATATTTTTATCACCCCAAAAGAAGCCCCATACTCTTTAACCATTAACCACATAACCATCAATAATC
ACTTCCTATTACACCCAGCCTCTGACAAAAAATACTCTACATTTTGTTTCTGGGGATTTGCCTATTCTGG
ACATTTCATAAGATGGAATCATATCGTATGTGGCCTTTTGGCTCTGTGTTCTTTCACGGGGTTTTCATCA
CATCCATGTTGTAACATGTACCTATACTTCATTTGTTTTTATTGCTAAATACTAATTCAGTGTTTGAATA
TCCCACATTCCTTTATCCATTTATTCATTGGTGAGTAATGAGGTTGTGTTTACTTTTTGGCTAGTATGAA
TAATGCTGCTGAACATTCATGTAAACGTTCTTGTGTGGACATATGTTTTAAATTCCCTTGAGTCTGTACC
AAGTAGTGGAATTGCCGAGTCATAACTTACTCTTTACCTTTCAGAATCATCCTACTTTTGAATGTGTATA
TGAAATATCCCTTATTTTTCACTACATTCTTTAAAAACCTTTATCCTAACTTTGGTTTCCATCAAAATAC
AGGCATACTTCATTTTATGGGGCTTTACTTTATTGCACTTCAAGATACTGCATTCTTTACAAGTTGAAGG
TTTGTGGCAACCCTGCATTAAGCAAGTCTGTCAGCGTAATTTTTTCAACAGCATGTGCTCACTTCATGCA
TCTGTGTCACACTTTGGCAATTCTCACAATATTTCAACTTTGTAATTGCCATTATTTAATTATTACTGTA
TGTGTTATGGTGATCTATGATCAGTGATCTTTGTTGTTACTGTGGTAATTGTTTTGGGGTGCTATGAACC
ACATCCACATAAGACAGCAAACAATCATTAAATGTGACGTGTTCTGTTTCAGCATCCCTATCTCTCTCCC
TTTCCTCAGGCTTCCCTGTTCCCTGACACACAACGATGTTAAAATTAGGCCAATTAGTAACCCATTCCCA
CCCCTAATGTTCAAGTAAAGGAAGAATTGCGCATTTCTCACTTTAAATCAGAAGCTAGAAATGATTAAGC
TTAGTGAGGAAGGCATGTTGAAAGCCAAGATAGGCCAAAATCTAGGCCTCTTGCACTAAGTACTTAGCCA
AGTTGTGAATGCAAAGGAAACATTCTTGAAGGAAATCAAATGTGCTACTCCAGTGAACACATGAATGATA
AGAAAACAAAACAGCCTTATTGCAAATATAGAGAAAGTTTTAGTGATCTAGATAGAAGATCAACCAGCCA
CAACATTTCCTTAAGCCAAAGCCTAATCCACAGCAAGGCCATAACTCCAATTGTATGAAGGCTGAGAGGT
GAAGAAGCTGGAAAAAAAAGGTTTGAAGCTGGCAGACGGTACCCCATGAGATTGAAGGAAAGAAGTCACT
GCCATAACATAAAAATGCAAGGTGAAACAGCAAGTGCCAATGGAGAAGATGCAGCAAGTTATTCAGAAGA
TCTAGCTAAGATCATTGATAAGAGTGGTTACATTAAACAACAGATTTGCAATGAAGGTCTAACAGCCTTC
TACTAGAAGAATATATTATAGCTAGAAAGGAGAAGTCAATGTCTGGCTTCAAAGCTTCAACGAACAGGCT
GACTCTCTTGTTAGGGGATAATGCAGCTGGTGACTTTAAATTGAAGCAAATGCTAATTTATCGTTCAGAA
AATCTTAGAATTATATTATTTAATTATTCAGAATATTAATAATTATTTACTATTCAGAAAATTACATAAT
AATTATATTATTTACTATTCAGAAAATTACATAATAATTATATTATTTACTATTCAGAAAATTAAATAAT
TATATTCTTTACTATTCAGAAAATTAAATAATAACTATATTATTTAATTATTCAGAAAATTAAATAATAA
TTATATTATTTAATCATTCAGAAAATCTCTTAAGAATTATACTAAATCTACTCTGCCTGTGCTCTATAAA
TAGAACAACAAAGCCTGGATGACAGCACGTCTGTTTACAGCATGGTTCACTAAATATTTTAGGCCCAGTG
TTGATACTTATAGCTCAGGAAAAAAAAAAAAGATTTCTTTCAAAATATCACTGCCTATTGACAATGTGCC
TGGTCACTCAAGAGCTCTAACGGAGAGGTACAAGGGCATTAATGTTATTTTCATGCCTGCTAACGCTATC
CATTCTGTAGCCTATGGGCCAAAGAGTAATTTCAACTTTTAAGTCTTATTTTTTAATAAATACATTTTAT
AAGACTATAGCTGCCATAGATAGTGATTCCTCTGATGGGTCTGGGAAAAGTAAATTGAAAATCTTCTGGA
AAGGATTCACCATTCTAGATGTTATTAGAACATTAATGATTTGTGGGAGGAGGGCAATATACAAACATTA
ATAGGAAATTGGAATGAGTTGATTCCAACTCTCATGGATGACTATGAGAGGTTTAAGACTTCAGTGATGG
AAAGAACTGCAGATGTGGTGGAAATAGCAAGAGAATTAGAATTAAGAGATGGAGCCTAAAATTGTGACTG
AATTGCTGCCATCTCGCGGTAACTTGAATGGATGAGGAGTTGCTTCTGATTGATAAGCAAAGAAAGTGGT
TTCGTGAGATGGAATCTTCTCCTGGTCAAGATGTTATGAACATTGTTGAAGTGACAGCAGAGGATTTAGA
ATATTACATAAGTTTAGTTGGTAAAACAGCATCAGGCTGGAGAGTGGTGCTACCAAAGGCATTATCATAA
AGACTTTTAAAAGAGGAAAGTTGAGTTGAATTGCACCAAAGGTAAATATAGACGAAAAGAAGGGGAATTT
AGGAAAATTTACTCAGAAAATTGTCATTTTTTACTTTAAAGCTATGAAGCGTAAACTTCAAATTTCTGAG
TATAAAGATGGTTAGAAGGCACGAAAGATAAGTCCCGCCCATTGCCTGACACATAGTAATCCCTTAACAA
GCTGATAATTCTAGAACATGTTACCTTTTGTAGTTGAAACTAAAAATCTGTTTATGTTGTTATTATTAGT
TTTTAATGGGCCTTTCTTGGCAGGCAAATAGTTAAGTCTTTATTTCTTTGTTTCCATCCAGGCCTCTTAT
GTGAGGAGCTGAAGAGGAATTAAAATATACAGGATGAAAAGATGGCAATGTTGCCTCCCCCAGGACCTCA
GAGCTTTGTCCATTTCACAAAACAGTCTCTTGCCCTCATTGAACAACGCATTGCTGAAAGAAAATCAAAG
GAACCCAAAGAAGAAAAGAAAGATGATGATGAAGAAGCCCCAAAGCCAAGCAGTGACTTGGAAGCTGGCA
AACAGCTGCCCTTCATCTATGGGGACATTCCTCCCGGCATGGTGTCAGAGCCCCTGGAGGACTTGGACCC
CTACTATGCAGACAAAAAGGTGAGTTTATTTTGACTTCAGTGGTCAGTTTCTGTTGGCTTCCTTCTGTAT
AAAAATTATTTTAAATTGAGATTAGTGAAATTTAGTGAATAACCATATCATGACCAGAATGTTAAATTAA
ATATATATATATATATATATATATATATATATATATATATATATATAGGATATTCAGAATCTGTACTTTT
TCAAATTTTTCATTGAAATTGGCTCTGGCATTGGCATACTACTTTTAATTTTTTTTTTTAATTATACTTT
AAGTTTTAGGGTACATGTGCACATTGTGCAGGTTAGTTACATATGTATACATGTGCCATGCTGGTGCGCT
GCACCCACTAACTGGTCATCTAGCATTAGGTATATCTCCCAATGCTATCCCTCCCCCCTCCCCCCACCCC
ACCACAGTCCCTAGAGTGTGATATTCCCCTTCCTGTGTCCATGTGATTTCATTGTTCAATTCCCACCTAT
GAGTGAGAATATGCGGTGTTTGGTTTTTTATTCTTGTGATAGTTTACTGAGAATGATGATTTCCAATTGC
ATCCATGTCCCTACAAAGGACGTGAACTCATCATTTTTTATGGCTGCATAGTATTCCATGGTGTATATGA
GCCACATTTTCTTAATCCAGTCTATCATTGTTGGACATTTGGGTTGGTTCCAAGTGTTTGCTATTGTGAA
TAATGCCACAATAAACATATGTGTGCATGTGTCTTTATAGCAGCATGATTTATAGTCCTTTGGGTATATA
CCCAGTAATGGGATGGCTGGGTCAAATGGTATTTCTAGTTCTAGATCCCTGAGGAATCGCCACACTGACT
TCCACAATGGTTGAACTAGTTTACCACCAACAGTGTAAAAGTGTTCCTATTTCTCCACATCCTCTCCAGC
ACCTGTTGTTTCCTGACTTTTTAATGATTGCCATTCTAACTGGTGTGAGATGGTATCTCATTGTGGTTTT
GATTTGCATTTCTCTGATGGCCAGTGATGATGAGCATTTTTTCATGTGTTTTTTGGCTGCATAAATGTCT
TCTTTTGAGAAGTGTCTGTTCATGTCCTTTGCCCACTTTTTGATGGGGTTGTTTGTTTTTTTCTTGTAAA
TTTGTTTGAGTTCATTGTAGATTCTGGATATTAGCCTTTTGTCAGATGAGTAGGTTGCGAAAATTGTCTC
CCATTTTGTAGGTTGCATGTTCACTCTGATGGTAGTTTATTTTGCTGTGCAGAAGCTCTTTAGTTTAATT
AGATCCCATTTGTCAATTTTGTCTTTTGTTGCCATTGCTTTTGGTGTTTTAGACATGAAGTCCTTGCCCA
TGCCTATGTCCTGAATGGTAATGCCTGGGTTTTCTTCTAGGGTTTTTATGGTTTTAGGTCTAATGTTTAA
GTCTTTAATCCATCTTGAATTGATTTTTGTATAAGGTGTAAGGAAGGGATCCAGTTTCAGCTTTCTACAT
ATGGCTAGCCAGTTTTCCCAGCACCATTTGTTAAATAGGGAATCCTTTCCCCATTGCTTGTTTTTCTCAG
GTTTGTCAAAGATCAGATAGTTGTAGATATGTGGCGTTATTTCTGAGGGCTCTGTTCTGTTCCATTGATC
TATATCTCTGTTTTGGTACCAGTACCATGCTGTTTGGGTTACTGTAGCCTTGTAGTGTAGTTTGAAGTCA
GGTAGCGTGATGCCTCCAGCTTTGTTCTTTTGGCTTAGGATTGACTTGGCGATGCGGGCTCTTTTTTGGT
TCCATATGAACTTTAAAGTAGTTTTTTCCAGTTCTGTGAAGAAAGTCATTGGTAGCTTGATGGGGATGGC
ATTGAATCTGTAAATTACCTTGGGCAGTATGGCCATTTTCACGATATTGATTCTTCCTACACATGAGCAT
GGAATGTTCTTCCATTTGTTTGTATCCTCTTTTATTTCCTTGAGCAGTGGTTTGTAGTTCTCCTCGAAGA
GGTCCTTCACATCCCTTGTAAGTTGGATTCCTAGGTATTTTATTCTCTTTGAAGCAATTGTGAAATTTAG
TATAATGTTTAGTATAACATAGTATACATTGTTTAGTATAATTGTTTAGCATAACATTTTTGGACATGAC
ACAGTGATCTGCCAAATTTTATAATCATACCTATTTTTTCCTGCTAATAATTAACTCTCATGTGTATGCA
GACCCAAAAATCACTTAAAAGTTTTTAATGAGAATGAAAATTGCTAATTTCAATTTTAAAATACCAACAA
TTCTTTTTATTAAAATTTCAAGTATAAGGTAACTTGATCTTGCCAATACTATGTAGTATGTTGTGATTTT
TCATGTTAGCTCAGATGACCTCCGGTTTATAAAGTCTCCCTGATAATTTTGACAGTCATGTGACCCTGTT
CCCTCTTCACTCTCATTCTTCCCCCTTTATATTTTTCTGTATGATATCTGTCACATCTTGCCTTATATTA
GTTACTTTCTTCTTTTCATTTTTCTAGGAAGTTCCTTGAATACAAAACTCATGCCTTATTTTTGTTCAAA
CTCCTTTTCATTACCTGTTATGATATAGTTTCTGACTATACATGTATTAGTCTTTTTTTTTTTTTTTTTT
TTGAGACAGAGTCTCCCTCTGTTGCCCAGGCTGGAGTGCCGTGGCGCGATTTTGGCTCACTGCAAGCTCC
GCCTCCCGGGTTCACGCCATTCTCCCACCTCAGCCTCCAGGGTAGCTGGGACTACAGGTACCCGCCACCA
CACCTGGCTAATTTTGTTTTTGTATTTTTAGTAGAGACGGGGATTCACCGTGTTAGCCAGGATGGTCTCG
ATCTCCTGACGTTGTGTTCTGCCCACCTTGGCCTCCCAAAGTGCTGGGAACACAGGCGTGAGCCACCACG
CCCGGCCGTATTAGTCCATTTTTACACTGCTATAAAGAACTACCTGAGACTGGGTAATTTATAAAGAAAA
GAGATTTAATTTACTCACTGTTCTGAATGGCAGGGGAGGCCTCAGAAAACTTACAATCATGGTCGAAGGC
AAAGGGGAAGCAAGGCACATCTTAACATGGTGCAGGAGAGGGAGAAAGAGGAGGGAAATGCCGCACTTTT
AAACCCATCAGATCTCATGAGAACTCACTCACTCACTATCATGAGAACAGCACTGGGGAAACCGCTCCCA
TGATCCAATCACTTACCACCAGGTCCCTCCCCTGAGACGTGGGGATTACAATTCAAAATGAGATTTGGGC
GGGGACACAGAGCCAAACCATGTCAATACAATTGCAGAAAGAATGGATGAATAATTTTTTAAAAATACAG
TCTACTGAGATTTAAGACAAAAAGGATTGTACTTGAGGACTAGGTATTAATTTTAAAATAATTTTCCATG
TAAAAGTGAATTTTACATTAAATTGTATTTAGATAGAACTGATACAGTGAAAGGGAGCAGGAAAATAAAA
GGACACCTGAAAACAATAGACTCTGATTTGTTTTCTTGAGGGAACGCTGCCGCATACAAATAAAAAGCCT
GACTCACAGACTTACCTCCTGAATACGGTGAGAAATGCTTATTGCCAGAAACCCACTGATAACCCTATAT
AAAATTTAGAAAGTCAGTGTCTTTCAGCTAACACAAGTCATCTTCCTGCCAACCGGAGAAAAATAAGGCT
GGGATTTGAACTTTGTCTTTCTGATTTCTAAGCCAATGCTTTAGGTTGCTTTAATATTTTGTTGTTACTG
CTGGTTGTTTTTATTTCTTAACTGAATACTAAAGCTATGGAAAAGGACACTTATGGAATGGCTACAATAA
CATTTCAAGAATGTTTGCTTCTATTCATATCGTTTTTTAAATTACCGTGGTTAGTAAGTGAGGAATCCTC
TTAATTTAAGTCCCTTTTAAAAGCTAATATTAGTATTATAACTAAAATAAAGAAATTGAACTCAGTGATT
GGAAGGATTCTTCCAACTCTGACGTTCCTTCAACTTAGGAAAATATTTTTTGAAATAACTCATATTTTGG
AACATGTGCTGCTAACCATAACAATTGCCAGCAAGTATTAAAATTCTCAAGGTTTTATGATAAAAATATT
AGAGATAGCTAGTTTCTATTTGGCTTTCTTAAAATGCAGTGTAATGGAAGGGAAAGAAGTTGTACTTTAA
AATAACTTAACCACAGTTATTGTTACTGGTGAAAGAAAATGAAAAATATGAAATACAGTATTTTGAAAGC
TCTAAGCAATACATGCTAACAAATAGCTCAATTTTTAAAGTTACTATGAAGAAGTGGACTTGGAGTTCAT
TGGCCAAGGTGACATTGATAGATGCGTTGATGACATTGGAACCACATTGCTGGAGTCTGATGGCAGTGTT
TCTTCAATATAACACTGCCATGTAGAAAGGTAAACTGCTGATATTGATGTGAAAAATTGATATTTTGGAT
TCTCAATTTCATCCTTTCTTTTTCCTCCTGCAGACTTTCATAGTATTGAACAAAGGGAAAACAATCTTCC
GTTTCAATGCCACACCTGCTTTATATATGCTTTCTCCTTTCAGTCCTCTAAGAAGAATATCTATTAAGAT
TTTAGTACACTCATATCCTTTTAAAAATGATTACATCCAGTGGCACTTTATGGTGTAATTTTTGCTATTT
TATTCAAATATAGATGTTATACCTTTTGCACCTTGAAAGACTCTGGTTTTAGTTTATTAATTTCAGTATT
ATTAATATAATTTGTCATGGGTCTCAGAGAAGACTGGGAGAGTGGCAATATTAAGATTTTAGGATATGTT
ACTTTTATTTATTACTCTCTTGTTCAAAGAGACAAAATAGTCTACAAGCTCATTTAGAATTTTTTTCTTA
GGAAAAGTTGTAAGCATTTCAACAAGTGTCATCCTCAAATATTTCAAATTCCCACTGTCGTCTCTTTTGT
TCCTTGATTCTAAGCTACCTTATTCAGCATGCTCATCATGTGCACTATTCTGACAAACTGCATATTTATG
ACCATGAATAACCCACCGGACTGGACCAAAAATGTCGAGTAAGTGGGTATAAGTACATTTTAATATAGTT
TTGGTATTATCATTTCATCCTTTCCTTTTCCTGCCAGGAAGTTACTGCATTTATGAAGATGGGGAATCTT
TACCTTCCCATGTTATATGCTTGCTTTTCTTTTGTTCTTTACCTCCTCTATGATATTAACCTGCTAGATT
AGAAAAATTAGATGCTACAGTGTTTTCTCAAAGAATGAACTTGTCAGATGCTCCATCCTGTTCTTAGACA
TGTTACACATATTTTATTCTTGGGTGTATGTTTTATAATTATATGGGTGTATTTTTAAATGATTGGGTGA
ATTTTTGATAGAAGCAATGTAGGAAATTTTTAGGCAGATCTGAGACCTATATTCTTAGTTATTTCAAGTA
CCTCTGCTTTCTGGGAGTGTTTCTGATATTGACATTTTAATATTTTCTGGATAGACCTATTGTAGTTTAA
TAGGGTGTGTCCATAACCCAACAGCCATCCTCCAATATATGTCTTCCAGTGGTTAATTAGAGAAAATAAG
TTATAAAGATTTACATGGTGGTTGTATTCTTTTCACATCTAGTATCCCAATGGAATCTTGTGTTTAGGTA
CACTTTTACTGGAATATATACTTTTGAATCACTTGTAAAAATCCTTGCAAGAGGCTTCTGTGTAGGAGAA
TTCACTTTTCTTCGTGACCCGTGGAACTGGCTGGATTTTGTCGTCATTGTTTTTGCGTAAGTACTTTCAG
CTTTTTGAAACGGCAAATTTATGAAAATCTCAGGCAGCACAGTGTTCCAATAACAGCAAACCTCTGGGGT
CTGATTTTTCCAGCATGGAATGTCTGTATTTAAAGGAAAATGGAAGCTATATGAAAGCTAGTCATTGATA
GGCAAATACTAAATTTTTCAAAAAGACTATCTCCCACTCAGAGGTATGTGACATCATTGAGATTGATCAT
CTTCCTCCATGCTCAGCACTTGGATGGATAGTCTATGCAGTGCCAATTAGGTTTACACTTTTTCCAAGTT
CTTGTTTAAAAATGAATAAAACCTATAAGAAGCACACAAAAAACTCAGGCCTTTAAATACAAGTACAGTG
CTCTTTATATTACCCTGTATTATTCAATTATTTTATATTTTTGTATTAAAATATAAAAGTATATATTTGT
AAAAGAGCTGTCACTGCTTACTAAGCCTAAGATTAATGCATTGTATACACATTATATATACACTCAGTTT
ATTTTCAAGGAAAATATTACATTACAGTATAATTTTAATTTATTCATAATTGTTACTCTTTTCCCAAATG
TATTTATCAGAAAAATGGATTTTTACAATCTCATATCTAGATATTCTATTTTCCGTTAATGACTTTGAAT
TATCAAAGTAATCCAGGTTCACTGGAGCCACCTAGACAATACAGAAGGGTCTTAAGATTCCTAATATCCG
ATTCCTTCATCCCTGCCTAACCTCTTTTCCATCAGGCTCCCTCCTTTTGAGGTCACATATGATGGGGCAG
TTCCATCTGCTACCTGCTGCTAAGAAAACATTGTGCAAAGAAGACTGCCTCTTCTCATAGGTCTCCTTTT
TTTCCTATTGGTACAATTCAAATACTACAAAATTCACAATTTTAAAGTATACAATTCAGTGTTTTTCAGT
ATATTGACAAACTTGCGCAACATCACCACTACCTAATTAGAGAAGTTTAACAACCCCAAATAGAAACTCT
GTACTCATTAGCAGTCACTTCAATTTCCTTCTCTCCCTGGCCCCTTGGAAACCACTCATCTATGTTTTAT
CTCTATGGATTTGTCTATTTGGATTTTTTTATGAATGGAAATATCAAACAGTATGCAGCCTTTTGTGTCT
GACACTTAGCATACTATTTTCAATGTTTTATAAAAAATTAAAAGTCTAAATTACCATAATGTTAAGGGTC
CAACAATTGAAACATTTAGTTGTTTACGCATTTAAGTGTATGAATCCAATTTAATAATATATAATATCTG
TATTTTGTTTGCATCAAATGAGTCAAATCAATGCTAATTAAATTAAGTTAGTTATGATAGCAAAAAAGAC
TTGCAGAACTATACATTTAAAATAGAAGCCCCAAACGTAGAAAATACCTTGACTAAAGGCTCAGGTAAGT
ATCATAGACTCTATCTAAATTCTGAATAATTCTGATTTAATTCTACAGGTATTTAACAGAATTTGTAAAC
CTAGGCAATGTTTCAGCTCTTCGAACTTTCAGAGTATTGAGAGCTTTGAAAACTATTTCTGTAATCCCAG
GTAAGAAGTAATTGGTGTGAAGCATTAGGCCACTCATAACTCCAACTATTTGGGAGTTGTTCTTTGTTCG
TTTGTGTGTGTGTCGTCACTGTGTGTATAAACTCCCCTATTACAGATATGTGACAGAGTTTGTGGACCTG
GGCAATGTCTCAGCATTGAGAACATTCAGAGTTCTCCGAGCATTGAAAACAATTTCAGTCATTCCAGGTG
AGAGTGGGGTTAAACACCAGGGCTGACTTTGATCTTTTGAAAGAAAGACATAAAAAAAACCTTTCTTTAT
GCAATTTTAATTAAATTTGATTTCTTCTGTTTTCCACAATCATTATCAAAAGTCAACCTTTGAGTTTAAT
CAATTGCATGGGTCTTTAGGATGAGGATGACTACTTGGCTCACATCTCAACTTGAAATCTTCCCTCCTGT
CTGAGGGTTTGTTTTTCCATTTGCTTATACAGGAAGGAAAATTTCAATCCATTTTAATTGATTTTAAAAT
ACTTAGGAAAGAGATCATAAATATCAGCATGGGGATATTGCATGACATTGTTTTTTATTTTTTCTCAGCA
AACCATTTCTTTATTGAAAATTATTACAAGGTGTCACTATATGCTGTAATTTGTTTTGGTGCCATTTATA
GAGAAGTGGTTTGTTTGACAAACATTAGAACTTACAAAAATTATTTTTAAAGTGGTTTGAATAGCCAAAT
TGTTCATTTTGACAACCTAGGTATTCTTTGAGAATATGTTCCTATTTTTAACCATTTGCTGTAATATATA
AAAACAAAGTATATATAAAATAGCAAACCCAGAAGGCTTTTTTTGAATTAGGTTACTTAAGGTCATTGAT
TTGATATAATGCATGACTTTCTAGGAAAGCTTGTGTTTTTTGTTTCGATTCAGAGGCTTTATGTCATTAC
AAACACTTTTTTCTCCCATTTTCAGGCCTGAAGACAATTGTAGGGGCTTTGATCCAGTCAGTGAAGAAGC
TTTCTGATGTCATGATCCTGACTGTGTTCTGTCTGAGTGTGTTTGCACTAATTGGACTACAGCTGTTCAT
GGGAAACCTGAAGCATAAATGTTTTCGAAATTCACTTGAAAATAATGAAACATTAGAAAGCATAATGAAT
ACCCTAGAGAGTGAAGAAGACTTTAGAAGTAAGAATGTCCTTGCATTTGTTATTAGGTTGAAATAATGCT
AAAAACATTGCTCTCTTTTAATAATGAAATGTTGCTTTCATTTTAATCATTTCAAAGCTATTCAAATATA
TAGAAAGCTGCTACAGTGTGAATTTGGGTCATGTTATATATGATTTTCATAAATCATCCTGGTTCTAAGC
TATCTAGCAGTACTGGCAAAAGGAACACAGGATAGTTCCATAATTCCTAATTTCTATATCATAAAAAAGA
ATTATTTTCTCTAGAGAAATAAATGTAGCCTTGAATTAATAACATTAACCATTACATTTAACAATATAAT
AGATTATATATTTTATATAATTTTATTATATAGTACATACTTATATTTATTATATATAATGTGACATATT
TATTATGATAGATTTTTATACTTAATAGATTGTACTCATAACTATAACATCTTAAAGTTCTATTAATCAT
ATATAATGATAATATTAATTATCAAATATAAAGATAGTATCTGTATCTTGATAGTATTCCTAAAGATTTG
TACAAAGCAAAATCATTTGCTCAGCTTTATCAACATCCACTTTGTAACCAATGACGAGTTTCATAGCTGT
TTTTAATGGCTTCCTTCAATATAGGCTGATGGCAAGATATCAAAGTTTAGTTCAGTAGAATTATTTCTGT
AGGTACCACATGAGATCATCTGTATGCACTCTCAGAAGTCTGCTTAATTCAGAAGTATCAATTAGAGTTT
CTTACATAAAGCATGAATTGAATGTTCTCTAAACAATTATCCCCTAGCATGGTGTCTCTCACTGGAACGG
TTGATGAATATTGGATGAAGATATGATCAAGATTGTACTTTCTGTCTGACACCTGGAATGTATCTATCTG
ATGGTGCCCACGTGAATACATAAATCTTAATAGATAATTTGACCCAGGTGTGGTATGGAAGTCACGAAAC
CTTTCAAATATATGTGCTGTTGCCAAAAGGAAATGAGGGAGGAAAGGTCAGGATTCTCCTTAGATAATAA
AAAGTAATATACATCATTTTCTTATTTTACCAAATGTGTCCTTTCTCACTCACTGTGATAAATAAAATCA
AATAACACCTAGCCCACATGGTAAAAGACTATAAATAAAATGACAGAAAAAAATAGAAGCCTTAACAAGA
AAACTGAAAGTTTTGAAGTAAAATGTTTAGCATATATATTTTTACTACCACCATTTGATGAGGAATTTAG
CATATTTTCCCCTTCTTTTTATATTTACCCTCTTTTCCAGTTTCTGATTGTTACAAACATGATTTGAAAC
TTATTTCCAATGGATGGAGAATTTTATTTCTTAATTTATATGCTGTAAAATTAATATATCTGATGAGTAA
CTTACATTTAATATTGTAGATTATTTTACCACACTTATTTAAACTTAAGTTTTCTCACTGGGATCTGTCC
TTTTATTCTGAGTCTTGCTCCTATTTCTGAGTTTATACTGTGAATGGTTTTCTTAACCTTTGTTAAAAGA
AAAATTTTCAACAAATTAAATTTAGCAGCTTATTTCAGCAATGAAATAGTATTTATTGCTGAAATAAGAT
AATAATAATAATGATTTAGGCAGCACTCAGAACCAAGAGAAGTTCGGAGAGCTCAACTCAGCAACATGAG
CAGGAGTATTTATAGACAGAAAAAGGGAGTGACGCACAAAAACAGCTTGATTGCTTATAGCTTGACATTT
GCCTTATGTGGACATTGTCTGATAAATCAGCAGGCTGTGGTTGGCTGAAGCTCAGCTGCTGTGATTGGCT
GAGACAGCTACTTGTTACAAGAATATACTAATAGATTAGAATGCAATTTGTTTACATATGAAGTTAGATT
GCAATTCTCTATGTACACAGACAGCCTTTAATCAAATTTAATTTAATTCATCGCTTTCTGGAATTGCTTC
ATGTATTTTTTTTTTTTTTTGCTAAAAAATAATACAAAAAAAGAAGAAAAAAAAAGAAACCCCGTCTCTA
CTAAAAATACAAAAAATTAGCCGGGCATGGTGGTGGGCGCCTGTAATTCCAGCTACTCAGGAGGCTGAGG
CAGGAGAATCGCTTGAACTCGGGAGGCAGAGGTTGCGGTGAGCCGAGATCGTGCCATTGCACTCCAGCCT
GGGCAACAAGAGCGAAACTGTCTCAAAAATAAATAAATTAATAAATTAATAATAATAATAGAGCCCTTGA
GTTATGTGAATTATCTTTATATTATATTTAAACATGAATGCATGACAATTAGCTAATAATGAATCTTATA
CCACAATCCTGTCTTCCAAATCTGTACATTATTCTTTATTTTCTTCTGAAATGTAGTTTTAGTGAAGAGG
TGTTTAAATCAAGTCTGAATTTTGCTTCTGTTTCCTATGTCATAGAAGCCATTTTTGACTGCATAATTGA
TTCAAACTTTCATTTATTGGTGCGTTTATTACATGGTTTCCCTGAGCTATAGCAATAAATGATAGCCTCA
GCCCTTGCTTTCACAGAGCTTATCATCTAGTGGGAATGAAAAAAAATGTTTAAGGGGGAACAGACAAATA
AATTAAAAATATCTCAAGTTATAATAAATGCCATGCAAAGGAAGTGAGTCTCAGGTGCTGAGATATCAAC
TAGATGAGAAGACATCTCTGAGGTGATGGCATTTAAGCTGAGATCTAAAAGATAGTAAGAAGCCATAAAA
TAGTTTAAGTGTGGGTGTGAGTCTATGTGTGGGGGGTGCTGCTTCAATTAGGAGAAATAGCATGGGCAAA
GGTCCTGAATAAAGGGAGGGTATGGGTTTAAAGAATGGAAAAACAGCAGCGTGGTTTGGTAAGAGAAAGG
AAGAATGGCAACAGAGAAAGTAGCAGAAAAAATAGGGGTCATATCATGCAGGACATTTCAGCCCATGGGA
AGGAGTTTGTATTTTATTTTAAATGCAGTCATTTTTATTGCTGTCTGTCTTGTGCACAACTGATTGAGCA
AGGGTAGATGGTAGATGGGAATAAGAAAGGTAAGTTGAAGGTTGCTTCTCTCATCTAGGCAAGAGAAAAT
AAAGGTTTCATCTAGAGTGATGGCAGAGAAATGAAGAGAAAAAAGATGAGCTCAACATACAAAGTCTGTA
GAGCTTGCTGATGGATTAAAGAAGACAGGAAGCATTGAGGATGACTTCTCAGTTTCTGGCTGCAATAACT
GGATGGGTGGTGATGGTTAATGAGGAAATGGTGGACACATTTGTAGAGGAAGGTCACTATTAGCTAGCTA
AAATTAAAATGAGATGCCTTTGAGATGTCCAGGTGGATATATAATATAGAGAATTGGAGATTTAAGTATA
GAAATCAGAGGGCCTGCTGAGTTTGGAGATAAATTCCAGAGCAATCAGCAAAAAGAAAGGACACACACAC
CTCTACCTCTGAACATCCCTGATATTTTTAAGTAGATAGAGAATGGGAAACCTGGTATTTAAAGCAATCT
GGTACCAAGATCTACAACTCTGACATTTAGAATTCAGGTAGAAAAGAGGACCCAGAAAAGAACACAGAGA
AAGAGCCGCCAATAAAGTAAGAAGCAAAGATATAAATAATGAAGCCAAGAGAAGAAAACATATTGTAGAA
ACAGATGTGCTTGTGTAGAATCATGCTGCAAGATCAAGGAGTAAGGATGATGAAGAAAGAAATATGCATT
AGGTTAAGTAACAGGGAAGTCATCAGAGAATTTCTTGAGAAAATTTGGGTAAGTGGTAGGGTTCAAGGTA
AGAATAAGGTGCATACAGGGGTGAAAAGAAGAGGTCAGAAAGAAGGTAATATGTGTAGACAATTCTCCCA
AGAAATTTGGCTATAAAGGGAAGCAGAATAATGGGGCAAATGGCTGGCATTTAAAAAAATATATGGGTTT
TTGTATGGATGTTTTGCTAGTGATAATGAATGAGTAGTTATATATGAATCAATTCAGGAGAGAGAGGGAG
AAGTCAAGTCCTCGTGAAAGTGAAAGAGTAAAGGGGAGCGACAGCTCCACCGAAACAGGAGGAAAGATAG
AAGAGAAGACTGCAGAGTTATCAAAAGATGATTGAGTTCAAACCCAATGGTTTTTATTTCCTCAAAGTAT
AAGATGGGATCATTGCCTGAGAGTGCAAATGAGGAAGAGAAAATTCTACAGATGATTTGATGAGAATGCA
TTATATATATGGTCATATCAGGGCATGAGAAATTTAGTGTATTTTGAGTGCAAATTTGATGTTGGAGATT
ACTTACTTAAAATATAACCATTCACTTGGTTGTGAGAGTTTCTCCACCAACATTCAGCCATTTGGTAGCC
AGCATGGAAAAGACAGATGCCAGATTATGTGTAGACTGTGGTTCTGCCAGGTTAGGCACTGATGATAATG
AATGCCCATGGATTTAAGCTGTACAAAGAGTGACAAGAACTTGGTTGATAGATCATGAGAAAGTGCTAAG
GCAAATGAATTGGCAATATTTAAAAGATCAAGGAATTATAATGGTAAAGGCAATCTACACAGAGAACCTG
AGCATAATCTAAAAGCATTTTCTGGTGCCTATAGCAAAGTATTTTCCGAAATGCCTTTCCAAATTCACAA
TCAAATAAATTAACCCACTGTCTCACCTCCTGCTGAAAAACATACAATTTGTAGATTTCCTTTGGTGTTG
CTGTCTACTTTGATGCTCATCAAACACTTTTTTATGCTGCTACAGATGAAAGTGGGGTTCATGTGCACAG
TGTGTAGCTAAATGTGCAGTATTATGGAGAAATATACTGTGGTTATATTGGGCTAAAACAGGATATTCTC
TTCCTCCTGTTTTTCTAATATTTGACATTTAATAGAGAAAGTTTTGAAAAAACCGCAATATTGGCCTTCA
CTACTAACAGGAATTTTCCTATCTCCTTCATCTACAGCTGTTTTCTTGTGAGAACTACATTTGCCAGAGT
GTAGAGTATCATGTGCAGGCCCTCTGCCCAGTCCTGATCCCCTGCACTAGTTTTCTCAGAGTTGCATTAT
TTGCCCTTCGTTGGGGATATACTCTTCACTTCCATAGGCCCAAGCCATACACAATTGTCATAAGTTGTCT
TTCTGAGCAAGGGCAGAATTACCATTTTTTTTTCTATTTTGCTACATTAATCTTGAAAATTAATTTAAAA
ACTTCACCCAGAATCTTCATTAGATTTAATATCAATTCTGTATTTTTGTTATATCAATTTTCATTTTCCC
CATACGTATTTTAGTGATAAGTTGGCATTTATTTACCCCCACCCACAACCTTTGCCGTTATGCTGCATTC
CCTTTTAAAAATAGGTTTATTGAGGAATAATTTATATGCCACAAAATTCCTTTTTTTAAACTGTATAACT
CATTTTTTAAACTATACTCAGAGTTGTGCAACGATCGCCACTATCTAATTTAAAATATTTTCATCTTTCC
CACCAAGAAACCCAGGGCCCGTTAGTAGTCATTCCCCATTTCCCTTTTCTCCCAGCCCCTGGCAACCACT
AATCTACTTTCTGTCCCTAGGGATTTACCCACTCTGTAGATTTAATATACATGGAAGCATATAATATATG
GCTTTTTGTATCTGGCTTCTTACACCTAGCATAATGTTTTCAAAATTTATCCATGGTATAGTATGTATCT
ATGCTTCATTTCTTTTTATTGCTAAATGATATTTCATTGTATGTATATGCTATATTTTGTTTATACATTC
ATCAGTTGATGGACATTTGGGTTATTTCCGTCTTTTGGTTATAATGCATAATGTTGCTGTAAACATTCAT
GTACAAGTTTTTGTGGAAATACATTTTCAATTTTCAGTTCTCAGTATATACCTAGTAGTGGGATTACTGA
GTCATGGTTCTTTTTTTTTTTTTTTTTTTTTTTTTTTTTTTGAGATGGAGTCTCACTCTGTCGCCTAGGC
TGGAATGCAGTGTCGCTATCTCGGCTCACTGTAAGCTCCGCCTCCCGGGTTCACACCATTCTCCTGCCTC
AGCCTGCCAAGTAGCTGGGACTACAGGTGCCCGCCACCACGCCCAGCTATTTTTTTGTATTTTTAGTAGA
GACGGGGTTTCATTGTGTTAGCCAGGATGGTCTCGATCTCCTGACCTCGTGATCTGCCCGCCTCGACCTC
CCAAAGTGCTGGCATTACAGGCGTGAGCCACTGCGCCCGGCCGTGATTCATTCTTTATCTTTCAGAATCA
TCCCACTTTTGAATATGCACAAGCAATATTTCATTTTTCTTTGCATTCAATTTTTTCCCATTTTTAAAGA
TGTTAACCTTAAATCTTCTTGAAGCCAACCATCTTAATAATTTAATGTCTAGTTGTTTAGAAATTATTGT
AAAGTTTTCTACAAACCTAGAGTGAGCTACCTGCATTGCTTTACCTTTCATCATTCTTATACTGTGTTAA
ATACAGTAGTATAGCAGTGTGCTAATAGACAGCTCCAGAGCCCATTCATATAATTCAGCTCCTCAGTGGT
TGATAAATGTGAACCAGCATTAGTTTAAAGGATAATTCTAAAGTAATCCATCCTTATTAAAATATTTTGG
ATGTTTATGTATGCAAAATTGCATCTTATCCTCTCCCATACTCACTCTACCAGGCCCATCCTTCCCGAGG
TTCAGGAGTGTTTTCAGGAAAGCTTTTCATGCACATTTTCACCTACATTTGCACCTCAAACTGCAAGGCT
TTATTGTTTTATCTGTGTCTTCTAAGTAAGATAAAATGATGATGTGTAATTCAATCAATAGTTTTATCTC
AAATTGTATAATAGTTTAACTAATTATAATAACTCCTGTTTTTGAGCAGCTAGTACAGAATGTGTGGATC
ACAGTATCAAGTGGTTTACATGCAATATCCTATTTATACTATTTAACAACTTTATAAAGTAGGTATTCTT
ATACGTATTTTACAGATGCAGCAGCTAAGGCTTAATGGCTTAAAGATAATTCACTTACTTAAGAAGGGAT
AGAAGCAACATTCAAATCCAAGAATATCTGATTTCATAGTCTTTCTTCCATGATTACTCCAAAAGTCTCC
CAATAATCAGATTTTTTAATTTGACCTGAGTAAAATAGGAAGTCAGGTTCTTATAGTAGAATAGATATTT
AAGAGAGATGACAAAAGGTTTTTTAGTCATTTAAACATTTACTATACAAGGATAGATCTGCCCCCAGTCT
TAGGAACAAGCCTTAACAGCAAATGACAGAAGAGTTGATGAGAACTTTTACTACTAAAAATTAGCACAAA
GAATACCATGAAAGGTGAAACATGGAAAATCTGCATTGATTACATTGTCATCTCCTCAACACCTAGAATT
AGTAGGTCCTAAGTAAGTATGGACTGGTGAATGAAAAGTAAGATAATTTTAGAAAACAAATTTTACTCAC
TTATGTTACTGCCTTACTGAGTCTGCCTCTTCCGGAGTATTGTACCACATAAGGATGTTTTAGTCAATAG
CGGACCATATATATAATGGTGATCTCATAAGATTATAATACTGTATTTTTTTACTGTACCTTTTCTGTCT
TTAGATACACACTTTCCATTGTATAACAGTTGCCTACAATATTGAGTACAGTAGCCCACTCTACAGGTTT
GCAGCCCAGGAGCAATAGGCTATACCACATAGCCTAGGTGTATGGTAGCCTATACCATCTAGGTTTGTAT
AAGTGCACCCTAGGATGTTTGCACAAGGACGAAACGTCCTAATGATGCATTTCTCAGAATGTATCACAGT
CATTAAGTGACACATGACCGTACCTTTAATCATACTTAAAATCCAAACTCATTTCTATGATATGCAAATT
CCGACATGATCCGGCCCCTGCCTACCTCTGCCATCACAATCCTCCCTCTCACCACACTGTAGCCACACTG
GCCATCTTGCTGGCTTTTCCACACACCAACTTCATCTCTGCCTTAGGGCTTTAGTTCTTTCTCTTCCCCC
AGCCCAAATATCTCTTCACCAGTATTTTCATGGCTGTCTTCTTTGTCACACTTAGGTCTTAGTTTAAATA
TCATCTCCTTAGAAAGACCGTCCCTGACTGCAGCCTACCACGTTGCCTTAATTTATTTTCTTTAGATCAT
TTATCACTATCTGAAAATATCTTGTATATTTATTTATTTACTTAAATTTTTATCCAACCACAACCTCCAC
CCTCACCTAAACATAAAGGAGGTCTCATAAAACTAATGATTTTATTTATCGTGTTTATTGTCATGTTTAT
TGCCTAGCCTCTAAGAGATTATCAATAAATATTTGAGGGAGAATATATGAGAAAATAGTAAATAAATGGA
TGCATATTGCCTGGGACCAGGCCTGAATTTGTAGATAAATGTCTATATCAATTAATTTACCTCCTTTATC
ACAATCACAGATTAAAGTCTGTGATGTTATAACTGTTCAAATTCTTCTTCAACAGAATATTTTTATTACT
TGGAAGGATCCAAAGATGCTCTCCTTTGTGGTTTCAGCACAGATTCAGGGTATGTAATATTTGTTTTCTT
TTTAGTCTAAAGGCTGAAAGAGAAGGAAAAGAATGTTCAGTCAGTTTGCAAATTAGTCTTTGATTCTATA
TTGCTTTTTATATATTTCATGGTATTATTTGACTCATTCTGTCCCACAGCAAATCCAGTCTTTAAAATGT
TCACAGTTAATGCATACTCTTGAAAGCAAGACTTTGAGACAGTGCTCGAATTAACACAAGACAGAAGTCA
TATAAATCTTAAGAAGGTAGAAAATGTTACTTCTAGAAAACTCCACCAAAAGTTGGTTGGATAATTTCAG
GTCTTCTTTCTGGAAAACGAAGAGTCACAAAGTCTATTTTTCTTTCTACTCCCAAGTTCCACTCATTCTC
CAATTAAATGTGACTCATAGGTGACTGAATGTCATGTTTTGGCCACTCTAAGCAAAGGGATTCCTTGGAC
TTGGGTACACATATTTTAAGAATATTGTATATATAAGAATATTGTATATATTCAACTGTCCATCTTGCTG
CTTCTCTCTTACACCAACTTCATTTTGTAGCATCAGCTGCATAAGCTCTTGCTTTTCCCCCTATGCAGAC
ATCTATTCATCAGGCTCTGTGTACACTGGCAACATAGGAATAAACTGGATAAGGTTGCTGTTCCCAGAAG
AATCTCAGAATATTGTATTTATTTAATGGCATCAGGAAAAACCAGTCCACCAATTTTAGAAAGAAAAAAG
TCTTAAAGCTGCACATTTTTATGAACTTAGACAATTACTATATTTTGACTATTATTGACTCTAATCTATT
CCAAGAATTATACAGACAGAAAGAGCTTTGGTAACAGCCTACATATCAATATTTACTGAACCAAGAACTA
TCACAAAACGTCTGTTGAAAATTTCCATGTTTTAATGATGATGATGCTAATGGCAGCTATGATTTATTAA
ACACTTTCTATGTGCAGGATGTAAGCTAAGTAATTTGTACACATTATCTTATTTAAACTTATTACAAGTC
TTATTTTCTTGAGGTAGATGTTATTATCCTATTTTCACAGACAGACTTATGCCTTGAAGAATTTGGTAAA
GTCACTAAGTAGTCAGTAGCCAAATCATGTATTCTTAATCCTTATTCAATATTGTCCCCCTATAGAAGAA
ACCTTGAGTTTGTAACTTTTCAGTAAGGCTATTTAGCTTGTGTCCTGAAGACACTCTCACCTATAATGTT
CTTTCTCGTGTGTAGTCAGTGTCCAGAGGGGTACACCTGTGTGAAAATTGGCAGAAACCCTGATTATGGC
TACACGAGCTTTGACACTTTCAGCTGGGCCTTCTTAGCCTTGTTTAGGCTAATGACCCAAGATTACTGGG
AAAACCTTTACCAACAGGTGAGTACCAAGAGAAACATGCATTGTATTTTTGAATGGCATATGTACCTGGT
GTATGTTAAGAGCCTGTATTAGGAGGTTTTTTATTTATTTGAGAATGGAGGAAACTCTATTATCTTATTA
CCAAAATTATAAGTGTTTTACTCCCATCACTAATAATATGGGTAGTTCTCGAATCATAAATAATTTTCTT
ATCTTTTCTGGGCATATTTAATCTCTAGATCTCTCGCCTTCCTGAGTATATCATCCCATGGGGTCCTAGT
TAAGTACCAGGACATACTGTAATGTTTTCTCAAGCTGCTCCTGCACATGGACTGTTTTCCATGCGTGCTT
GGGATGGCCTGAAGAGAAGAGGCTCTCTGATGTTCACCTGCTCACCATGTTTTATTTTTGAACTCCTATT
TAAAGTTAAAGATGCAATTCATTTCTCTAAGTTTTTCTCTCCCAAATCAATTGCTCCCAACTGTAGACTT
CCATGATTTTTTGTATTTTCTTCCATTTATATCATTGATCTCTTGTATTGCAAGTGGTAATTCATGTGGC
TATTTCCTCTAACTTGAATGTGAACCTCTTGGGGCTGTGGTCTCCATTTACTTGTATAGTCTCAGCAGCC
AAGCCAAAAGGAAGGAGACCAAAATGATTATTCTATAGTACACATAACAATGATTCACTTTTCTCAGAAT
AGCTGCAGATCTGTTTTTTTTTCTTTTTTGGCCTATTTTATATTTACTTTATTTCCGAGAATATTATAAA
TGAATTCGGAAATTATGTAAGATGCACTTTACAGCAAAAATGGGAATGCAAATATTTTTTAAATATGCAA
GAAGTGGTCACCTTTACTCAAAATTAAAGAAACACAAATAAAAAAATTAAAATAATTTTTCATCAGAGGT
AAGACTTATTTTTCATTTAATATCAGTTGATACTATCCTAAATTCACTTTAAAATGCTTTGTATGATAAA
AGTATATTACATTATACAATTATTAATAGAAATGTTAATTATCTTATAAATTTGTTTAAGTTACTTGTAG
ATTCTTAATATTAGACCTTTGTCAGATGGGTAGATTGCCAAATTTTTCTCCCATTCTATAGTTTGCCTGT
TCACTCTGATGTTAGTTTCTTTTGCTGTGCAGAAGCTCTTTAGTTTAGTTATATCCCATTTGTCAATTTT
GGCTTTGGTTGCAATTGCTTTTGGTATTTTTGTCATGAAGTCTTTGCCCATGCCTATTTCCTGAATGGTG
TTGCCTAGGTTTTCTTCTAGGGTTTTCATGGTTTGGGGTTTTACATTAAGTCTTTAATCCATCTTGAGTT
AATTTTTGTATAAGGTGTAAGGAAGGAGCCCAGTTTCAGTTTTCTGCATATGGCTAGCCAGCTTCCCCAG
CACCATTTATTATATAGGGAATCCTTTCCACATTGCTTGTTTTTGTCAGGTTTGTCGAAGATCAGATGCT
TGTAGAAGTGTGGTGTTATTTCTGAGGTCTCTATTCTATTCCATTGGCCTATATGTCTGTTTGGGTACCA
CTACCATGCTGTTTTGGTTACTGTAACCTCATAGTATAGTTTGAAGTCAGGTAGGCTGATGCCTCCAGCT
TTGTTCTTTTTGCTTAGAATTGTCTTTGCTATATGTGCTCTTTCCTGGTTTCATATGAAATTTAAAGTAG
TTTTCTAATTCTGTGAAGATGTCAATGGTAATTTGATGGGACTAGCATTGAATCTATAAATTACTTTCGG
CAGTATGGCCATTTACATGACATTGATTCTTCCTATCCATGAGTATGGAATGTTTTTCCATTTGTTTGTG
TCCTCTCTTGTTTCCTTGAGTAGTGGCTTGTAGTTCTTGAAGAGGTCTTTCATATCCTTTGTAAGCTGTA
TTCCTAGGTATTTTATTCTCTTTATAGCAATTGCAAATGGGAGTTTATTCATGATTTGGCTCTCTGCTTG
TCTATTGTTGGTGTATAGGAATGCCTGTGATTTTTGCACATTGATTTTGTATCCTGATACCTTGCTGAAG
TTGCTTATCAGTTTAAAGAGTTTTGGGGCTGAGATGATCGGGTTTTCTAAATATAGAATCATGTCATTTG
CAGAGACAATTTGACTTCCGGTCTTCCTATTTGAATACCCTTTATTTCTTTCTCTTGTCTGATTGCCCTG
GCCAGAAATTCCAACACTATATTGAATAGCAGTGGTGAGAGAGGGCATCCTTGTCTTGTGCCAGTTTTTA
AAGGGAATGCTTCCAGCTTTTTCCCATTCAGTATGATATTGGCTATGGAGTTTTCATAAATAGCTCTTAT
TATTTTGAGATATGTTCCATCAATACCTAGTTTATTGATAGTTTTTAACATGAATGGATGTTGCATTTTA
TTGAAGACCTTCTCTGCATCTATTGAGATAATCATGTGGTTTTTGTCATTGGTTCTGTTTATGTGATGGA
TTACATTTATTGATTTGCATATATTGAACCAGCCTTGCATCCCATCAAAAAGTGGGCAAAGGATATGAAC
AGAGACTTCTCAAAAGAAGACATTTATGCGGCCAAAAAACATGAAAAAAAGCTCAACATCAGTGATCATT
AAGAAACGCAAATCAAAACCACAATGAGATACCATCTCAAGTCAGTCAGAATGGTGATTATTAAAAAGTT
GAGAAACAATAGATTCTGGTGAGGCTATGAAGAAATAGTAGCACTTTTACACTGTTGGTGGGAGTGTAAA
TTAGTTCAACAATTTTGAAGACAGTGAGGCAATTCCTTAAGGACCTAGAACCAGAAATACCATTTGACCT
AGCAATCCCATTACTGGGTATATACCGAAACGAATATAAATCATTCTACTACAAAGACACATGCACACAT
ATGTTTATTGCAGTACTATTTACAATAGCAAAGAGATGGAATCAACTCAAATGCCCATCAATGTTAAATT
GTATAAAGAAAATGTGGTACATATACACCATGGAATACTATGCAGCCATTAAAAAAGAATGAGATCATGT
CCTTTACAGGGACATGGATGAAGCTGGAAGCCATCATCCTCAGCAAACTAATACAGGAAGAGAAAACCAA
ACACTGCATGTTCTCACTCATAAGTGGGAGTTGAACAATTTGAACACATGGACACAGGGAGGGGAACAAC
ACACACCGGGGCCTGTTGGGGGGTGGGAGGCAAGGGGAGGGAGAACGTTAGGACAACTACCTAATGCATG
CAGGTCTTAAAACCTAGATAATGGGTTGATAGGTGGAGCAAACCACCATAGCACATGTATACCTGTGTAA
CCAACCTGCATGTTCTTCACATGTATCCCATAACTTAAACATATGAAAAAAAGCTCATCATCACTGGCCA
TTAGAGAAATGCAATTCAAAACCACAATGAGATACCATCTCATGCCAGTTAGAATGGTGATAATTAAAAA
GTCAGAAAATAACAGATGCTGGAGAAAATGTGGAGAAATAGGAATGCTTTTAAACTGTTGGTAGGAGTGT
AAATTAGTTCAACCAGTGTGGAAGACAGTGACCCAGCAATCCCATTACTGGGTATATACCCAAAAGATTA
CATATCATTCTATGGTAAAGACACATGCACACATATGTTTATTGTGGCACTATTCACAATAGCAAAGACT
GGGAACCAACCCAAATGCCCATCAATGATAGACTGGATAAAGAAAATGTGGCACATATACACCATGGAAT
ACTATGCAGCCATAAAAAAGAATAAGTTCATGTCCTTTGCAGGGACATGGATGAAGCTAGAAACCATCAT
TCTCAGCAAACTAAAACAGGAACAGAAAACCAAACAACGCATGTTCTCACTCATGAGTGAGAGGTGGGCA
ATGAGAACACATGGGCAATGAGAAGATCATCACACACTGGGGCCTGTCGGTGGTGGGGGGCAAAGGGAGG
AATAGCATTAGGAGACATACCTAATGTAGGTAACAGGTTGATGGGTGCAGCAAACCACCATGGCACGTGT
CTACCCATGTAACAAACCTGCACGTTATGCCAAAAAAAAGAAAAAAAGAAAAAAAAATCTCTGTACCTTC
AGTTTAATTTTCCTCTAATCCTAAAACTATTTTGAAAAAAAATTCTATTTTAAAAAGCAAAGAAAGCGAA
AAAAAAAGTTAATTGAAAATACCTATGAGATGAATAAAGTAATTAAATAAGACCATCTGCCTAGTGTTAA
CATTCAATGATATTATTAATATTTGATGATAAAGAAAGAAAAGATAGAATTTTAACAGTAAAAGGGACTA
TGGATATCACCTGTTCCTAAAATTTGAAAGTGAGCAAAGGATTTAATTTTATGAGTAATTTTCAGACTGT
AGGAAAAAATGCATGATTTTTATACTACTACACTTAATAATCAAGAATCTTACATGACTTTAGTACAAGC
ATATTACATTGCTATATTCTTCGTACTGAAAATGTGTTTAATATTTGTTTTCCATGAAGATATAACTTTT
CTCTTTATATTTTTGATAACATGAGATTATACCAAACTGTCAGATTTGCTCATGCCTGTCAAATTGAAAT
AATTTACTAAAAAAACTTTTTATTGTTCAAATGACAATTTCCATTTTTCCCTAGACGCTGCGTGCTGCTG
GCAAAACCTACATGATCTTCTTTGTCGTAGTGATTTTCCTGGGCTCCTTTTATCTAATAAACTTGATCCT
GGCTGTGGTTGCCATGGCATATGAAGAACAGAACCAGGCAAACATTGAAGAAGCTAAACAGAAAGAATTA
GAATTTCAACAGATGTTAGACCGTCTTAAAAAAGAGCAAGAAGAAGCTGAGGTACTGTTATTTGATTTAA
AATTCTTCCTAGAGGTAGAAATGCAAACGGTTACAACAGAGGCTCTGCGGTATATGCTTGGCCTTCTCCA
GCTAGATTTTCTTTAATGAATTTTTGTGTATAAACTGTATCCTCATTTCTGGTGTGGAAAAAGGAACCAA
TTCTGGGCTGAGCTAGAATTGATACTTTCCCCATAATCATCTCACTGCATGAAAAGTGATCTAAGGCTGT
TCCTCTTAAACCTTTTACCTACAATGACTTAGTGTGTGTGTGTGTATGTCTATATATACATACACATATA
CATATAAATGTGTGTGTATATATATATATATATATATATATATAAACACATGGAACTATGTGTTTTTTTA
AATTATTATATCATGAGGAAAAATCTTTTAGAATAATTGATTAAATAATATAGTCAGATATATAATATAT
ATATAATCTCAATTTATATATATATACAATATATATATGTATATATATACACACATACACACAATGGATT
TCCCCAGAGTACCAATTGTATACAGTGGTTTCATGAAAATTATTTTAACGTTAAAATTAACAGATACTCT
TCTATGGGGTATGACTAAAACAGTGCATCATGTTTTGTTAATGTCACAAGGATTTTTAAGATAAAACATG
AGATATTAAGAAAATAATTTTGGTGTGGCTAATTTAGGTTGTCATAGATTCTCGAAAGTTTCCTCCTTAA
TCCTCTTTCCAGAGAGATTCTTCAGTGTAAATGTTTGAGAAGGCTTGTACTAAGAGAAACATAATTTCAA
ATGTACTGCAGTTGCCTTAACATTTGTCTGAAGGTTTCACCTAGTTAAATCTCTTCAAGGAGTCTAATGT
ATTAGGGGAGGGCAATCATCTGCAATGCAGATAAATTCTGATGCACAAATATTCAGTGCAGCATTTTAAT
AATGGTAAAAATCAGAAAAAATTTAAATTTTGGAAAATTAGAAAATAACTAAGAAAATTATGAAAATTGT
ATATCTTAAAGTATTATTTTGCCATAGAACTATGTGTTTTTTTAAAATTATTTTATCATGAGGAAAAATC
TTTTAGAATAATGATGGATTAAATAATATAGTCCGATACAATCTCAATTTTAAAATTATACATACATATA
AAAAGAATGGAAGAAATATTAGCACCAATTATTTCTGGGAGGTAAGATGACTTTTTTTCTGTTTTTCATT
TATATCTTTATTTCCCATGTTTTTACAAGAATTTTTTAGTGTTTATAAACAAAACATTTTGTAAAGGGAT
AGTACTAGGTTATCCCAGGTACTACCTAGGGAATTCCCAGAAGTTTCATTTAGGCCAATAGTCATTGACC
TTACCACTTTCTTACAATATTATGCTTTAATAATTCATTTATTTTTCAAATAACTCAATCTTTATTAAAC
CATAGAGTGCCTGATTCTATTGATGATATGGAGTTAATTGCCTGATAATCAAATTGTCTGTCTTATTGCA
TGGTTTATGCGAGTTTTACATTCCAAAATTACCTTGTAAAACTTTAACATATAATATTTAATTTGTCTTT
ACTGTAAGCAAATTAATAACATGTAATAGTTTAATTCTAAAATACAATAGAGCTTTATTTCAACCTGAGA
CAAAAACCAAATATATACTTTGTAAGCATTCTGATCATTGTGTAAAGAAAACGATCATTTCATGTTTAAA
GGACATGATTATATGCACCATGTTGTTATGCTAATAGGTGAAAGATGTCCTGTCCTAGGGTTTCCTAGGA
TTTGGAAATGACTCATTTAAGTGTTAACGTCTTGGCCCAACCAGGCAATTGCAGCGGCAGCGGCTGAATA
TACAAGTATTAGGAGAAGCAGAATTATGGGCCTCTCAGAGAGTTCTTCTGAAACATCCAAACTGAGCTCT
AAAAGTGCTAAAGAAAGAAGAAACAGAAGAAAGAAAAAGAATCAAAAGAAGCTCTCCAGTGGAGAGGAAA
AGGGAGATGCTGAGAAATTGTCGAAATCAGAATCAGAGGACAGCATCAGAAGAAAAAGTTTCCACCTTGG
TGTCGAAGGGCATAGGCGAGCACATGAAAAGAGGTTGTCTACCCCCAATCAGGTACCACCCAAATTGCTA
AATGTGTATCACCCGAGGCAGAATGCTAGAGAAAAATGTCTTATTTTAGGGTAAGAGAACTCGGGAGAGG
ATAGTGAGTGATTTCATTGGTATGATTTTAGGTAAAAACCTAGTATCGCTAGGCATTTATGGAAAGATGT
ACACAGGTTTCCATTTGGGTTCAATCATTTTTGAAAGACCTTATAGTTTCAGAATTGTCAATATATTCTC
TAATAAACTAATTTTCAGAAGCAAAATGTTCCTGAGAACCATTGTAGAAATAAAGAGACCATTCACACTA
TAGAATAACTTTTATATTTGTATTTAGTCTGTGTTTTGTAATGCTTCATTGGTTAAAAACTAATCTTAGA
CTAGTTTCTCTTCTTTCTTAAACAGTATAGGTAATAGTATCACTCACCCATTTGGAACTTTGGAGAAGCC
TTTGATATCCTCCACCCCCAACCACATCAACTACTAATATGTAGTGCCATAGTCTGTTAATTCTGTTTAT
GTGGAAGTTCTCTTTCCCTTTCTCTTTTCACTGAAACCTTCATCTCTCAGGCCTCCCTGCTTATAATTCT
GCACAAATCCCCAAGATGAACCATTGCTTCTGTTTTCCTTAGGTCTTCCCTGTTTTCCATCACACAACCC
CACAAATCACTTTTCTAAACTAAAAATCTAGTTAAAAATCTACAAGATTCTCCTGATATACACAGTAAAA
AAAGATAGAGTCCCTGCCTTCAAAATTTCACTTGAACCTAGGTGATGATACAGAACTTGGACACATATAT
CAATAAAAATAAATATGACAGGAGCAGTCCAAGCTGGTTTTGAGGCAAAGACCTTACCATACTTTTTCCA
TTGTTCACAATAGTTGCCACTTTGTTCTTTACATAGTAAGCACTAATGTCTATTGAACTAGATCCTTCCA
AAGGATTTACACCCTGGACATGAGTCATATAAGCATGAATTGAATTTTTCCTCCTCTTTGGTATTGGGCA
ACTTCTTTTTAAATATTTAATGACTTTATAAATTAACTGGTGCTAAAAGCTCTCTTTAAATACACTTTAA
CTGAGCTCTTAGTATGTAAGGTGGATTTTCAATGCAAAGAACCATACAGCATGATTTCCAATTTTTTTGT
GAAGCCTGGGGATTGATATATAAAAAGTTGAATGACCAGTAGCAATACTAAGTTCTTTTTTTAAGTTATA
AGTTCTGTGCAGCAAAATGAATGGCATTAAAAGAATGGAGCAGTAACTCTAGGCCAGAATATTAGAGAGC
TGTATAGCAACAAATAATTTACCTGGTCCTGAAAGGATGAATTCTACAAATTGTTTCATCCCTTAAGTGG
AAGAAGTTTTCTTCTCACTGAATTGCTTCGTTTTTAAGTATGTTTCTTTTAAGTCAGTACAGAGCCACCT
GCCCTCAGGCCAGTGGGTTCAGTGGTATTTATATGAAGTGTACTTCTATCAGTAGGTGCTTCAGCAACCA
CGTTTTTTTTAATTTTTCTGCAGTCACCACTCAGCATTCGTGGCTCCTTGTTTTCTGCAAGGCGAAGCAG
CAGAACAAGTCTTTTTAGTTTCAAAGGCAGAGGAAGAGATATAGGATCTGAGACTGAATTTGCCGATGAT
GAGCACAGCATTTTTGGAGACAATGAGAGCAGAAGGGGCTCACTGTTTGTGCCCCACAGACCCCAGGAGC
GACGCAGCAGTAACATCAGCCAAGCCAGTAGGTCCCCACCAATGCTGCCGGTGAACGGGAAAATGCACAG
TGCTGTGGACTGCAACGGTGTGGTCTCCCTGGTTGATGGACGCTCAGCCCTCATGCTCCCCAATGGACAG
CTTCTGCCAGAGGTGATAATAGATAAGGCAACTTCTGATGACAGCGTAAGGACGTTTTACATAGCTCAGG
CATGGCTGGGCCCTTGTTCCTGCATCAGTCAGTCTCTCTGCTGAAGGGCCTTCTCTGGTCTTGTGAATGG
CTCTGCATTTTGCAATACTTTTTCTATAGGAACACACCTTTGATTCATTAGCCTAATCCTCATTCTATGT
GTTGTAGGCATCTAACTCAAAGATAAAATCCCATTTTTGTAAAAAGGTACTTGTCTATTAATTTTATAAT
TGAAGATTATAAATAATCTCTTTAGAGAACAATTTATGACGGATACATATATAGATAGATATCAGTACAG
AAAATGCAAGGATAATGCTGATATTAGTTAATAATAGATCCCATTTCTATGAAGAAATTTGGATTACTTG
CCAGCTACTGCTATCTTTTTCAGGTCACAGCTTAATATGTACCTATTCCAAGGAATCTTATTTGAGTTGT
TAATAACAGCAAACACATATTTAGTATTTATGACAACCCCATGAGATGATACATTATTATCTCTGTTTTA
CAAATGGGAAAATTGAGGCATGGGGAAATGAAGCAACTTGCCCCAATTTACTCAGCTAATAAATGGTAGC
TCCTAAGTTGAAACGGAGGCTGTCTGTTTCAGATCTTGTGCTCTTAACCACTCACTCTGCTATCTCTCCA
ATGTGCAGTAGGCTTCTTATCTTGAAAAAATTTAGAATGGCTATGTATTAGGTCCTCCATATAAATGAAA
CATAACTGTTTTCATTTTTAAAAAATATTTCATCTGGAGTCTATGAATCAAAACAAAATATCTGAAATGC
AAGCATTGACACAAACAAACATAAAAGCAAAACAATCATTTTACAGCAGTTCCAAAGGTCTACGAGTCAC
TAAGTGGTTAGAGAAAGGTGTCTGATTAGGCTTGAAAGTGAGATCTTTCTCATTCCTGGAGTTTCCAGGA
TATCTCTTTAGCCCACTCACTATGCTATGCTGCATTACCAATATCAGCAAGGGCTTTGACTAACCCAATA
AGCTTCAAACATATGCTACATGCTTATATATTTATATTTGCAGACAATGTAGTATTTCAAGTGAGAGTAA
AGTTCCATCAAATCCATGGCCACACATATATTTCAATTTGAATAAATGCATGTGCTTTTAGGTAACATGT
GACCTCTATGTGTGCATGATACAGCTTAAGCCCTTCTGCAAGGTATTTTTCATTATTGAATGAGCATGTT
AAATTTCTGTGGAATCAAACTTTGCTTGCATTTTGTTTTGCAATTATGTGTGTAATATGTAAAATACTAT
ATAATTTATATCAGATTAGGAGATTATTTCATATTTTTAATGAAGTTTCTTCATTTAAGAGTTTTTTCAC
CATAGAAAAATCAGTATCACAATAAATCCTAAAGTGAAAACTACAAAACTAGAGGAATTAGTGGATACTT
ATAAGATACAATGGAAATACAGTAAACATAAAATTGTGGCACAATATATGGTTTATATGAAAACACTGCA
TAACTAATAACGTCATCTCTATACCTGAAACATGTCTACATGGAAAATAATTTGTTTAATCTGACAAATT
CTTGTTTGACAGACTATAGCACAGAAAACCTGGTTTTCGAGACTTTTCAAAAAGTTGACTTTTTACAGAG
ATCTCTCAAATGGGTGTGTCTGTTCACATACACATAGATGCATAATACACCCATGCACATATCTATGCCT
GTATGTCTGTGTTTTGATACACATACATGCATGACTATACCTATACAACTATACAACTTTACAAATCTAG
AAATATATATTTAAAATATATACCACATACCACACACAAGCAGACAAACATGATAGATCTAGAGTTGTAT
AGTTGTTTGTGTATATATGTGTGTGTGAGTGTGTGTGTGTATTTCTGGCCTGCATGGAACCAACATAGTG
TTTTAAATTTGAATTAGTTTCCAAGTTTTAAAAATAAGACATTTTCTCACAAAAATAAATACACTTGTTA
TGCATGAGTTCATGTTTTTGCATGGCAACAATTTCTAGAGCTGAAGAGAGGTCACCTCTTTCAGATGTAG
CTTGCATTCTCCATTTCACTAATTGTTCCTTTCAAACTGAGTCATTTATCATCATATGCTCTGCTCAGTT
CATGAGGTCTTCCTTGGCAGTAAGAGACAACCATTACAGGAGCTTCATTAATGTTGTTCCCTCAATAAAA
AGTAATTCCAGAAATTTATCAGAGAATACTGAGATTCTACCCCTTTAGATACACAGCACTACAGCACTTT
CACTGTTTTCATGTAGGACTGTCACATGACAAACTATCTGAATAAATTGAACACTCAGAAAGGCAGAGAG
GTGATGATAGTGATGATATAACAAAAAAGCAGCTAGCTTTATTTTTTATCTCATAAATATTAATTCCTTT
AATCCTCACAAGAATATGAAGCAAGCACTATGATTATCCCCATTTTACCAATATAAAACTGAGACAGTTA
AGTAGCTCTCCTATGATATACAGCCAGTAAGTAGCAGAGCCATAATTTGAACCCAGCAATCTAGGCTCTA
CTTTTAATTACTCACTACAGCTATATAAGCAAGATTTTATCTTATACCTACAATTTATTAGGATTCTGTT
CTTAAGACATTAATTGATTTTTTTTTTAGGGCACGACCAATCAAATACACAAGAAAAGGCGTTGTAGTTC
CTATCTCCTTTCAGAGGATATGCTGAATGATCCCAACCTCAGACAGAGAGCAATGAGTAGAGCAAGCATA
TTAACAAACACTGTGGAAGGTATGTAATAATCTTCTTTTACTGTACAGATTCTTAAATAAAATTAACATA
AATCTGGTCAACCTTAAATAGGCACATGAAATTCTTAGGAAGAATTCAGAGACCATAAAATGGTTTTAAA
TGATGGTAAAGATAACCAATGGATACTGATGGAATTGTTTTTATGTGCTTAAAAATGGATAAAATTATTT
GCTATTAAACACAGACATTGATAACATGGCAAAAATGACTTTGCAAAATGAAAAAAAGAAAACCCACAAA
GACAGATGTTGGGTGATCTGACCTTTCAAAGATAAGTGATTTAATAGTAATTGATGCAGAAAACAAGGTT
AACCCAGAGCTACGTGATTCTGTCACTATATAATCTTACAAATTACATATTCGCTCAAAAATTGTCTCTT
CTAATAAGCAAAGATAGTCAAAGGGTGACAACTTTACAAACTGTGCTGGCTTCTACCCTCTCCCCTGACA
ATATGCCCCAGGTCCCACCTGGTCCCTGGGTCTAACACTTCACCCCCCACATTCCCATTGTGGGGGCAAA
GTGTCTACTCATCTCCTAAATAAATGGTGAGAAGTAAATGGGCTAATGGTGTGAAATGTCTATTTCATGA
GTCCTCCATAAATTATTTCTAGTTGCTTGGTTCTAGGATTGTTTTCACCTGTGAACTATTACAGCAAGAG
TTTTTTCTGGACAGAATAGTCTCATCTTATTGTTGTCATGGGACTAAAAACACAGGTGAAAAATATGCAT
ATTGGTGGAAAACATACATTTCTCTGTTAAGGAAACATAGTTCTCCTGTTTACCATCACATGTTAGGTGC
ATTTTTCTCTGAAAGATGCCCACATATGTGCTTGCAGTCATAAATTTCACTTCCTTACCCTCTCTTATGC
TTCATCTAGGCAACGAACATGTCATAATAGACTTACATAAGAGGCTATGCTTTCATTGAAATAGTACTGT
TAAATTTTCAAAATCTAAACTATAAACCTTGTTTCAATATTAGAATGCCTGACTGATTTGTATCTGGTTA
GGAGTGAAACAGACAAATGGCTGACTCCATGTTCTCTGCTTTTTTCTCCCAGAACTTGAAGAGTCCAGAC
AAAAATGTCCACCTTGGTGGTACAGATTTGCACACAAATTCTTGATCTGGAATTGCTCTCCATATTGGAT
AAAATTCAAAAAGTGTATCTATTTTATTGTAATGGATCCTTTTGTAGATCTTGCAATTACCATTTGCATA
GTTTTAAACACATTATTTATGGCTATGGAACACCACCCAATGACTGAGGAATTCAAAAATGTACTTGCTA
TAGGAAATTTGGTAAGTCTCTTATTGTGTGTTATGTACTCATAGTTTCTCTTTTTAGTTGTCATCATTGT
CATTTCATATTTTTTATGTCAGATGAATCTGCAGCCTCAGAATTATGCATATCCAGAACTTTCCATATGA
ATGGATGTGTAGAGTCAAGTTTCTGGTTATGTCTTTTAAAATAATTGTGCAGTACTGTTGAGGTCTACAA
ATAACAATACTATTTCTATAAAAGGTTACCATCTTGGGAGTTGATAATGATTTTTTCTGCCAGCATATTT
CTTGGGAGCAACACTTAACATTTTTGGTTCTTTCTCACACCTTCCTAATTACATTGCTGCTTTCTTGTAA
GTACACACCTCACATATTTTCAGAAAACCTATGGATGTATTGCTTTGACATAATAGTAGTGAAGTGTTTG
GGTCCTCAGAGTCCCAAAGTCCTGGGTTTGAATGCTGATTCTGCATTATATGAGTGATATGCCCTTGGAC
AAATATTTTAACTCTGTAGTATTTAATTTTATCATTTATAAAATAATACTTCATTATAAAATAATTATTT
ATAATATAATTGTAACTGTATTTCATTTATCATATTCTTGGGAGGATTAAATGCTTTCTGCTTATTAGCC
TGGCATATAGTAAATACACAATAAATTAAAGCTATCATCATCATCAACATCATTGTCATCAAAACTTCAA
AGACTTAATGAAACAAGCTTTAAAAACAACTATTTTGAGAAACCAAAATGGTTGAACTGGACATAGAAGA
CTCAAGGATAGGCAAGAAGGTGATCAACAGACTTCTGGAAGATATAAGATCCAATTAGTAAAATTGATGC
CAGAGTGATCTCTAAAATGTACAGTGCTGGTCACACTAACTCAGGGTAAACAAAATGCCAGTTTTTGACT
ATGGATGTGAGGCAGGCACCAGTTCATTTTTAGGGCCAGAGAATCTCAGCTTATGACTCTTCATAAACCA
TGAATAACTTTGGGTATCTTTGTCAATGTCTATATAAGATAATTTTGCTTTTGCTGTGGTCCCAAACAAA
TTGACATTTGAAGACCAGAAGTTCTTTTCTGAGTCTGTCTTTTCTTCTAAATTGTCTACAAATATTCCCA
ATGTTTTTCCCCATGTGCTCGCTGAGTTAGCATTCTTCTTCCCTTCTATTTATCTTTGGAAAAAGGCAGG
AATATCTAGTTTCTAATTGTTGGTGGAGATCCGGATCTTGCAAATAAGTACCTTCTCACTGAGACGACTG
CATCCCTGTGAACTCAGAAAGGATTTACCTGGTCACAGCTATTACCACAACCTCAAGTCCAGCTGCTCCC
TGCCAATCTTCTTGATCCTGCATTATTTGAAAATTGACTCCTGCAGTTATAGACCTAAACATTTCCCTTA
GAATCTTCTTTGGTCAGATTCCTTGAGTCAAAGTGTACCCAAAGGATTCATTTCGGTGTTGCCTTTTTAA
GTGAGTCCTTTGATACCAGTATCATGAATTAATGATATTTTGCAATGCACTTTTGTGTCAAAGCTTTAGA
AGTATTACTTTAGCTGCCACATTGTCAATTAAATACTTTTTTCTCTTCTGCTGATAAATCTTTAAAGAGT
GAGTCAACATGTTCTTTATTACTCTAACAGTAGTCAGATCAGAAAAAAAAAGACTGGCTCAAAATTTCAG
TATCAACTTATGTTTTCTTTTTATCTTCTTCCAGAACAACTTTTTGACCCACATAAATTCTTTGAAAATA
GTGACTGTGTCAATTACCAAATGTTTTACTCTTCTGAAATTTTAATATGCTATTCTAAAATGCTGTCTAA
AAATGATGAGCACTGACAGGACAGTTAGAGCTTTTCAACCTTTTGATGCTGATATGGGAGCCAGGGAAGA
GCAATACCTAATTTACAGGACTAAGTTAGATAAGAATATCCATTTATAAAGTATTTAGCAAACTGTCTGG
AACACAGTAAATGCTAAGCAAACAGTGATTATTATCATTGTGTTGATTTCCTGTTTTCTAATATTAACAG
AAAAACATTATTTTTCTCACTTAGGTCTTTACTGGAATCTTTGCAGCTGAAATGGTATTAAAACTGATTG
CCATGGATCCATATGAGTATTTCCAAGTAGGCTGGAATATTTTTGACAGCCTTATTGTGACTTTAAGTTT
AGTGGAGCTCTTTCTAGCAGATGTGGAAGGATTGTCAGTTCTGCGATCATTCAGACTGGTAAACATAAAC
TAAGGTTGCCATTATATTCTATAATAAAGGGGTATTTCTTTGGTTTTGCATTATTTTTAAAAACTTTTGG
GAATTTGAATCAAAAGGCAAGATTATATCTTGATATCTAGTAAAATAAAATCCATTCATTGACAAATGCA
CTTAAAGAACTAAAACCACATGAATATTTTTACAATTTTTGAAGACATGCTCAATTCTAATTTGAAGTAC
ACGCTAATATAATATAAATATTTTCTATTAATATAGTACAATAATTTATTTCAGAGTAAGAAGCTTGTGG
CTTACAGAATTTCAATGATTATATGTTCATAACTTTATGAATTTGGTAATTAAAGATCAAATTGTTATCC
CCCAAAACAATTGTTGTCTGATCTTAACAATATAAAAACAAATAAAGACTTTTTAAAGCACATTCCTCTA
ATATGCTAGATTTTATCTAAGTGTCGGCATGAGAAAATGTATGAAAGTATTAGAAAGTAGAATTAAGGAT
AAATTATCAATTCTAAAAGAAAAATATCACAAATTATGATAAGAGGTTTCTATAGCAATCCTGAACTAAA
AGTACATAGTTTCTTCATAATATGTGTTGTAATTTTTTGTGGCAAGTGGAACAAAATAACAGAGATATTG
AAAATTGATGAAAATGAAAAATAAGAATATTTTAGCTGGATAATAATGACAATATTATAGGACTTAAAGT
CTGCTTTACCCTTTGAACAAAAATCAAGATTGGAAACATTGAAAAGATTTTCATAGTGATTAACATTAAA
CTTTATATTTGCTTTTAGCTCCGAGTCTTCAAGTTGGCAAAATCCTGGCCAACATTGAACATGCTGATTA
AGATCATTGGTAACTCAGTAGGGGCTCTAGGTAACCTCACCTTAGTGTTGGCCATCATCGTCTTCATTTT
TGCTGTGGTCGGCATGCAGCTCTTTGGTAAGAGCTACAAAGAATGTGTCTGCAAGATCAATGATGACTGT
ACGCTCCCACGGTGGCACATGAACGACTTCTTCCACTCCTTCCTGATTGTGTTCCGCGTGCTGTGTGGAG
AGTGGATAGAGACCATGTGGGACTGTATGGAGGTCGCTGGTCAAGCTATGTGCCTTATTGTTTACATGAT
GGTCATGGTCATTGGAAACCTGGTGGTATGTAACCAGATGTTCATGCATTTTAATTTCTCTGTGGAAATT
ATTTTGTGATGATGTGATCTATTATTTTTATAATTTTATAATTAATTATATCTACAATTATTAATTGCAA
AAGAATAGTGATAATTTTAATACAAGAAAGAACAGGAAAGAGAACGAAATTTTGTATTAGTTTTCCTAGC
CAAATAACCCATATTTATATTTTTCAAAATCACTGCCTATTCATTTTAAGGCAAAGGTTTCTATCTTTTT
CTCTAATTTTGAAAGTGATGGCATATGCTCTCTTGAAGCTATACTAGTAAATAAAATATAATTAAGTTAA
TAATAACAAAAATGAAGTTAATAATGGACCAAGGCAAATGAAAGGACTGGTCCCGCACCTTATAGCCATG
TGTAAGACTAGCCTCTATAGCAAGGATCAGCACCTATGCCCCACAGACCAAAGCCAGTCCAATGCCTGTT
ATTGTGTGGCCCTGGGAGCTAAGAATGATTTTTACATTTTTAAGTGGTTAAAATAATCAAAAGAAGAATA
AATGACACATGCACATTATATGAAATGCAAATTTTAGTGTTCATAAACTTTATTCAAACATAGGAATGTC
AATTCATTTACGTATCTTCTATGACTGCTTTTACATTACAGTGGCAGAGGTGAATAGCTGCCATAGAAAT
CGTATGCCCAATAAAGTCTAAAATATTTATCATTTGGCCCTTTACGAAAAAGTTTTCCAGCCACTGGCCT
ATAGCATCGCGTGGACATAGCAATCTGCCTACTTTTTGAATTAGTGTTTTCTTGGCTTGTACTTTTTTTT
TATTTTTTTTCTTCCTTATACTGATTAGTGAAAACTTTCAAGCTTACAAGCAGGAGAAAATTGGGATAAA
ATGCTCTTCTATGAGCCACTCATCTCAATTCTCAAATCTGCCACGTTGACTTGTCACAGTTGGTCAGCTT
GACATCAAATCTCTCATCAGGTATCCAGAAGGTGGGATATGGTAACAGCCATATGGTAAGTGCCTTCACT
TCCCACTGAGTTGAAAAAAATTACTAAAATATTGTTGTCCTTGTAACCACCATAAAAAGAGGATTCAGAA
GTCAGGACAGAAACTTACATTCATCCAGTCACACACTAAATGATACTGTGCCAGACATACACAAAGGCAT
TTATGCAGCACAAATTCTAGATTTTTACTGCACAAAAAATATACATCCTGGATTACCATTCTATAATTGT
TGATCACCCTCAAGTTGTAGTAATAAATAAGCTTCCTAGTTTTATATAGCCTTACAAATTCCTTCCAATA
TGGAATGAAATGCACATTCTCTTTGTTTCTTCTTCTCTGTTTTGTATTTATAAGCTCCTATAGCAGGATT
TGCTTGTGTATTTTTTTTTTTTTTGAGATGGAATCTCGCTTTGTTGCCCAGGCTGGAGTGCAATGATGTG
ATCTCGGCTCACCCCAACCTCCACCTCCCAGGTTCAAGCAATTCTTCTGCCTCAGCCTCCTGAGTAGCTG
AGATTACAGGTGCATGCTACCGTGCCAGCTAATTTTTGTATTTTTAGTAGAGACAGGTTTTCGTCATGTT
GGCCAGGCTGGTCTCGAACTCCTGACCTCAGGTGATCCAGCCTCCGTCGCCTCCCAAAGTGCTAGAATTA
CAGGCGTGAGCTACTGCGCCCAGCTGTGCATTTCTTCTTACAAAATTTTGACATAAAATCGTGAAATTGG
GAAAGGTATAACTTGTGAAAACATGCCACATCAAAGCCAATAATCACAAATAATTTAAATGTTTATTATT
TATTCAGGCAAATTCAAAGGGAAAGGCATTCCCTTGGCTCTCACCACATTTTCCACTCAATATTTTTTAG
TATCTTCTCTGTGAAAAGAAAGGTATTTAGGTATTGTTTAGATAGGAAGGCATTGTTTAGGTATTATTTA
GGGTGGAAGACCAAAACCAATTACCCATGGTCTAATCCTTGTTTTTAAGATGCTTATTAGGGCACAGAAA
AGTGGAACACAAAAGTTATCAGACAAGGAAGAAGTGATGAGTTCTACCAATAAGGTTAAATAAAATGCTT
CTGACACTTAAAGAAAGAGTTCTAGATTGAGACTTAAGGGAGGTTTTTGAAGTGGGCCTTTGAACAATGG
ATAATATAATATTTGATCATGGACACTGGTGTGAGAGTTATGTTTTCCTTTGAAAGGCAATATAGATTTA
TAGTAAAAAACAGAGGGGATATATTGGTGAGTGTTTCATTCGTGTAGAATTAGTGTTAACTAACTCATAT
TATTATTCTTTAATTATTAATTCTTGGCAAAGAAAGTATACAGAAGAAAATTCATAAAGTTGATAACCAA
CTCTTGACTAATGGAGGAACTACCAATATCATAGAATTAAAATTTAAGGAAAAAAATATATTCCTGATAT
AGCCTTCCACATTAGAGTTATATGCTAAACTGGCTTCTCTTTTTTTATATTTCTCTAATTTATTCTTTTG
ATCATTTTATTGCCAGTTAATTGAAATGGTTAAGGACCATAGGTTTAGGAAAACATTTGGAAAAATAAGT
TTTAGTCAGCTCGGGTCTATATCAGGAAACTTGCGAAGACCATTAATCAAAATGAGACTTTGAATCAAGT
ATACATTTTATTAATATTTCTTCTCTTGTTTAACATGATAATACAAAGATGATACAAATGGACTTTATTT
TGATTTGTGATACCCACTAGGTTATAGAAATATTAGAGACTTATGATTTTTGAAGGAAAGAAAGTTGTCA
CAGCAGCTGTTCTCAACGAAGTGAGAACCGCATGAGCCACTGCACCCTGTTAAAATGGTTTAACGCTTGA
ACAGATTAAGGAGACTAAAGCAGTAAAATGGACCCTCAGATCTTCACCAATATTCCTATTAAAACAAATA
TACACAAACATGTATTTCATAGTGAAATATACTTTTTCATTGACAACTATTAAACCATTTCAGGGATTTT
TAGCAATTATCAAAATTGTTCACTATGATTCCCAGACCTCCGACCTCCAGAATTTTCTCCTAAATCAGAC
TTATTGAGACACACAATTTTAGCCTGCTGTCTTTACTTACATCTCAGAGAAATTGTTTTAGGTATGACCA
AAATGAGGGATGAGTACCTTCTCTTCAGTTAGGTCTTACTTCGATTTTTAAAACATGAGGTAGATGATTT
GGGCCATGAAAAGACTTACATCAAATGCCAATTCAATTTCCTTTTTTGTTTCCTGGATAAAACAATGTTT
AAATATAATTTACAATTTACATGTTTCCTGCCCTCTGTGAACATTTTTTTTTTTTACAATGTGGAGTTGA
CCTGCACTTCTACTTCAGTGAGAAAAAAAAATAATTAACTCAGAAATAAAACTTTGCTTCAAGAGAATAG
AAATTGTCCTGACCTTTTTTGTAATCTCTTCTAGGGCAAACATAATAGGCAATGAAGCCAGCCAATCATA
ATGCCATTATTGCATGTGAATCAGGTTCAAGGGGCAGGTCCTTTTCTGTGCTTTAAAAGTCACATTAAGC
AGTATTCTAGCACGCTTCTTGGACAGAATATTTTATTACAGGAACTTTGTAAGTATTATGTGTTCCATTT
GTTAGAACAAGTGACATTTATCTTCTGGTAAAGTGAACAAAGATCTTTAGCACAATAGAAAATAGTCACA
ATTGTTTTGTTCTACCTGTTTCTCACAGGTAATAAAGAGTAATGATAGATGAATGCTAGGAGAGTTTTTT
GGGTTCATAAAAGAGAACTTGATTAAGGCACTACTGCTACAGAAATGAACTTAGAAATGATGTAATGATA
ATGGTGAGGGATCATTCTCTCTGCTCCCCATGGTAATTCTTCCTGGGTATGGCTGACCCTCTGCTGCTGA
GTCTGCAGGGGCTAGTCTAGGGATACAGTGATGAGGAGTCTACCCTCGCCTCTGTCTCTCTCATGTGGTC
TTATCCGTGTGTGAAAAGGTGCAGAAATCTTCGTTGCTGGTTTGTATTGTGGCCTATATTCGGAAAAGTG
TACTTATTTTATTTTATTTTTTATATTTCCTGTCTCCCTATTTCTCTACCCCCTCTCCCCACCCTGATAT
AGGTCCTAAACCTATTTCTGGCCTTATTATTGAGCTCATTTAGTTCAGACAATCTTACAGCAATTGAAGA
AGACCCTGATGCAAACAACCTCCAGATTGCAGTGACTAGAATTAAAAAGGGAATAAATTATGTGAAACAA
ACCTTACGTGAATTTATTCTAAAAGCATTTTCCAAAAAGCCAAAGATTTCCAGGGAGATAAGACAAGCAG
AAGATCTGAATACTAAGAAGGAAAACTATATTTCTAACCATACACTTGCTGAAATGAGCAAAGGTCACAA
TTTCCTCAAGGAAAAAGATAAAATCAGTGGTTTTGGAAGCAGCGTGGACAAACACTTGATGGAAGACAGT
GATGGTCAATCATTTATTCACAATCCCAGCCTCACAGTGACAGTGCCAATTGCACCTGGGGAATCCGATT
TGGAAAATATGAATGCTGAGGAACTTAGCAGTGATTCGGATAGTGAATACAGCAAAGTGGTAAGAATGCT
TCATATACTTTGTGTTTCATATTAACAATTAGTATGAAATGAATGAAAATTTCATATTTCTATTGTCATA
AGTTCTTCTTAACATTGCATGTAGTGATGGATAGTTTTTGTGCGTGACTTTACACAACAGCTTCCTGAAG
ATTCATCTGGTAAAGTTTTCTTACTAGTTCTTAATAATTGATTTAATCTTACTTAGCTGTATGAGAAAGC
TCAAAGGTATCTTTGAGCCTTCCCATGCTTCATCCCATGTTTCCTGTCAGTTGAAAAGTTACACAGTCTC
CCTCCACAGTGCCTGTTTGAATCCACCTCTCCTTTTCATCACCTTTCCTTTGTACTCACTACTTTTTGCC
TAAACCATTTCAAGCAGAGTCTCAACTGATGCCTCTGCTTCCATTTTGCATTTACTCTAGTTTACTTATT
TGATTATTTTCGCTCATGCAGATTCAGTCCTAATCATTTCACTCTCATGATGTATCCATGATCCGCAAAG
TATCGCTCTCTCTCTCTCTCTCTTCTTTTTTTTAAAAAACGGAGACAGGTCTCACTCTGTTGCCCAGTCT
GGAGTACAGTGGTGCTATCATAGCTTACTGCAACCTTGAACTCCTGGGCTCAAGTGATCCTCCTGCCTCC
ACCTCCTGAGTTGCTGGGACTACACACCCATGCCACAACACCCAGCTAATTTTTTTAAAAAAGTTTGTAG
AGACAAGGTTTCACTATGTTTCCCAGGCTGGTCTCAAACTACTGGACTCAGGCAATTCTTCAAACTTTTT
CCCCAGTTGCTGAGATTACAGGAGTGAGCCACCACGCCTGGCCATCCCCAAAGTCTCGAATTATGTACTG
TGCACAAAGCCTTCACACTATGGCCCTAACCAGCTTTGACCAGCTCTATCTCCTAGTGCTCACCCATAGA
TTTTTACCTGTACCAAACCAATTTCCTTTTAGGTCATAAACACTCTCCACACTTTGCTTGGGATTTCCTT
CTCCAGTCAAAATCCTTCAAGGCATCACCCTTGTATTTATCATCTATTAAATTGTTAATACTTAAATCGC
AGTTGGGTCAGGGTTAAATGAATTAACACGTTAATACATGTAAAATGTTTAGAACCATACTCAATGTTCT
CAAAATGTTATTTATTTCATCCCTCCTAATTTCTCCCCAAATGGATGTAGTCCCTTCTCATTTTTGAACC
GCAAACAATTTTATACTTCTTCCTGGATTCAGTAATCTTAAATTTTAATTTTTTGAGAAATTATGGTATG
CATCAAATAGTACTGTAAATTCATTGAGAACAGGAACTCTTTAAAACCTTTTTTTGTGCCCTCCTCTCCC
ACCCTTACCTGCATATTTCTAGTGAGTCAAGGTATGTATTGAGTTTTATTTTTATTGTGAAATATAACAA
CCGTATTACAAATTACTTTTATGAGATTGCTTTCAAGTCTTAAGGCATCATTTAAAAATAATCTTTTGTT
TTCTCTAGCTCATGTTAGAAAGTATTTGACAGATATTTACCAAATATCTTCTATCTCCTTAACTTACTAA
GACTCCACCCTCCATGAATTTATAGTGGACTTGGAAAACGAGATTATATGGAATAATCATGACTGATATA
TATATATATATATATCAGTAAAATGCTCAGGAGGCTGAGGCATGAGAATCACTTGAGCTCCAGAGGCGGA
GGTTGCAGTAAGCCAAGATCGCACCGCTGCACTCCAGCTTGGACTACAGAGTGAGACTCTGTCTCAAAAA
AAATCAATACATAAGATTAAATTAAATACATAAGATTAAATTAAATTAAAACAAAATAAAATAAAATGCT
CTATATTTAATTGAGTGAATAAATGATTAAATAAATAAATCATGATATATGTTTCTATGTAGCCAAGGAC
ATTTTTTCCAAGACGGTAAAGTAAGGAATCAAATCTAGTCATCCCTCGATATCTATGGGCGAGTAGTTTG
AAGGCCCCCCACAGAGACTAAAATCTGCAGATGCTCAATTCCTTTATATAAAATGGCACAATGTTTACCA
TAAAACCTACACACATCCTCTCATATGTACACTTTAAATTATCTTCAGATTACTTATAATGTAAAAGTAA
ATCACTGACACAATGTAAATATTTGTTTTACTATATTTTTTATTTGTACTATTTTTATTGTTGTATTTTT
ATATTTATTTTTTCCAATTATTTTCCATCTGTAGTTGGTTGACTCTACAGATGTGGGACCCAGGGATACA
GAAGGCCAGCTGTATTTTATTTGTATATTTTAAAATGTTCTACATGGAAGAAGTAATACTCCCAAAATTT
TAAAACCGTGTTCATTTCAGCTTAAAGTTAGAAAATCAGATTCCACGTATGCCTTTTAAGGAGCAGGCAT
CTAGGTTTAGTATCATATGGAAAGGGAAACTAACTCAGTCACCTATTTTTTTCTAACTAACTCTGTCACC
CATTTAAAGAAGATTAAAATTATAAACTCCAATTGCCTGGCAGACAGTTGCTCTCAGAATAAAACTTGGA
TTCTAGGTGTTAAAGCACTTCAGGAGCTGGGGAAAGTCAACCAAAGACTTCTGAAATTATTTCTTAACAT
TTATGAGGAATTATTTCTTACCTGTTCATTTTTCTTTCATTTAAAGTCAATGGCTGACCTTTCATGGATC
TCCTTAGCTGCTGACAAAATTCTTATTCTTAGTATGTTGAGTTGCAACATTTTTAAGGGAACCTTATAGA
ACTTTCCATTTAGCACTTCATTTTCTAACTGAAAAAGCAGACTCATTAAAAGGAAGTGATTTTATTCACA
GTTATAAAGCTAGAGTTATACAGTTATAAATCTAGAGTTATAGAGTTATACAGTTATAAAGGCAGAGCCA
TGTCAAGAGTCCTAGGCTTGTGACTTATAGCTCAATGGAATTTCTACCTCTCTTGTCCTCTCTTTGTCCA
TTTGAAACACAAAACAAACATTCCTATTGGACTTAATAATTTGTCTCCCCTGGGGTATAAAGCCATGCAG
CTATAGAATTTATATATCTGTGGCATGCAGATGAAGGTGTGTTGTCATAATGGTTAGCTATCTTTTATAA
CTAGTGATTTTATTTGTATTTTATAACTGAAATAGATTATTTTAACCATTCATAAAATTACTCTGTCACT
TATTCCATGAGCCCTTCCGAAAGCGTATGAAATCTAGTGCCCTATAAACTGAGTTGTCTAAAAATCATTT
GATGTTAATATTCTAATATTCTTGATTTTCACTAATTTTTTTCTCATTACATAATGGTTATATCTATATG
ATAAAAGAGTGTACATTCTTACTCTATTACATTTCCATGGTATGCACAATTAAATATACAGGAGTTTGTG
GCTACCTTAGAGGTTGTAGTAACAGATACACAAGCTAGTATTTTTAAGTCATGTAGCATAGTGTTTGAAT
TAGCAAGCCATTTTAAAAAAAGTGTTTGTTGTCATCGATTTCTGCATCATTATTATTACTTAAGAACTCA
GGTTCTTAGTGCTACTGACTTCTAATTATGAACTTTGAAAGTTTACCCTTCTTCATAAAAAATTTTCCAT
GACGTATCTCAAAGTATGTCATATTAAGTATAGACAAACCTACCTGAAAATATAAGAATTGAGAAATAAT
GAAGGTTCAAGATCTAGAGGAAATTTGTTTGATAAGATATTAAGCTGTGGTCAACTATAAAAACCTTACA
TCTAAGGTCTTTCTTTCCAGCCTCTTCAGATTAAAATGTTTCCAGTTTAAATTCTGTCATTTTTGTTTTG
TTTTGTTTTCCCATTCAGATCACTAAAATAGTGTACATTTTATTTATTTGTCTAAAATGTCTTTAATCCT
CCTATGCTAACTTTTCCTGCTCTAATTCTCATAATCTTCATCTCCCACTTGAGTTCCCAGAGTCTTGAAC
TGTGAATTCCCTCTTTTGCAACTTTTATTTTTTTCGTATCTGAGATGCCTTTGAAATAGTGATGGTTGTG
TTTGTGATGAGCCTGGATGAGCCAAAGAGGCAAATTGAATTACCCAATAGAAAGTAACTCCTAATTTATA
TATTTATGTCATTAAGTCTTAGCCACTACCTTGGGATATAGTTAAGAATGCCACTTTTTAAGATAGAATA
TTTGCCTCAAATCTTAGCTTTTGATAAGACCTTGAGCAATTTACAAAAACATTTTTTTCAGTTTATTTAT
GTGTAAAATAGATTGTAATAAAGATTATATGATTCTCATAAATAATTAGACAAATGCTTATTGAAAAGTG
CTCAATGTACAATTATTATAAGTATGGAATTGAGTGCTTATATACACACTGTAAGTAGCTATTGGTTACA
GATTGAATTTAAAAGAGATAGCGTGATGAAGATCAGTGCTTCAGAATTTTAGTGTGTGAAAGACATACTT
GGGGACTTTATTACAAATGCCTGTTCTGGATCATATCCTTTGAATTGTTTGCCTAAATTTGGATGCAACT
TCAGAATCATTAACAACGTTTGCAGCAGCTACTGATCAATAGAGACTACTATGAACAATCATACTCCAAC
AAATTGGAAAACCTAATGGAAATTGATAAATTACTCAACACATACAACTTGCCAAGACTGTATCATGAAG
AAATGGAAAACTCAAACAGACCAATAATGAGTAACAAGATTGAATCAGTAATAAAATCTCTCCTAGCAAA
GAAAAGTCCAGGACCACATGGCTTCACTGATGAATTCTACCAAACATTTAAAGAAGAACTAATACTAATT
CTTCTTAAATTATTCCAAAAAAATCAAAAGGAAAGGAATTCTTCTAAATTCATTCTACAAGGCCAATATT
ACCCCAATACCAGAACAAGACAGGGACACACACACACGAAAAGAAAGCTGAAGGCCACTATCCCTGATGA
ACACAGATGCAAAATGCCCCAACAAAATACTAGCAACTCAAATCCAACAGCCTATCAAAAAGATTATTCA
CCATGCTCAACTGGAAATCGTCCCAGGGATGCAAAGATGTTTCAACATTTGCAGAACAATAAACTTTCTA
TATTATATCAACAGAATGAAACATAAAAACCATATAATCATCTCAATAGACAGAGAAAAAGCATTTTATA
AAATCCAGTATCCTTTATTTGATAAAAATTATCAACAAATTAAGTATAGAAGAAACATACCTTAATGTAA
TGAAGGCCATATGTAACAAACCCACAGCTAACATGATACTAACCAGGGAAGAGCTGAAAGCTTTCTCTCC
AAGATCTGGAACAAGATAAGGATGCCCACTTTCACTACTTTTATTCAACATAGTAATGAAGTTCTAGCCA
GAGCAATCAGGCAAGAGAAAGAAATAAAGGACATTTAAATTGGAAAGAAGGAAGTCAAATTCTCTTTGTT
TTTATATGATCTGATCTTAGATATAGAGAGTTCTAATGACCCCATGAAAAAAAGTAGTAGATTTCATAAA
TGAATTCAGTAATGTTGGAGGACACAAAATCAACATGCAAAAATCAATATTATTTCTATATGTTAACAAC
AAATAATCTGAAAATAAGAAAGCAATCCCATTTACAATAGCTACAAAAAATAAAATACCTAGAAATAAAT
TTAAACAAGGAGGTGAAATATATTTACAATGAAAACTATAAAATATTGGTGAGAGAAATTGAAGAGGACA
CAAAAAGGGAAAGATATTCCATGTTCATGGATTAGAATAATTATTATTTTTAAAATGTCCATATTACCCA
AAGCAATCTACAGGTTTAATGCAATCCCTATCAAAATACCAGTGACATTCTTTACAGAAAAAAAAAAAAA
AAGTCCTAACATTTGTATGAAACCACAAAAGACCCTAAATAGCCCAAGTAACCTGGAACAAAAAGAACAA
AGCTGATGGCTTCACACTAACTGACTTCAAAACATACTATAAAGCTATAGTAACCAAACAGAATGATACT
GACATAAAACAGACACACAGAGCAATACATAAGAATAGAGAGCCCAGAAATAAATCTACACATATACAGT
CAACTGAGTTTTGACAAGGTTCCAAGTACCTATAATAGAGAAAGGACAGCCTCTTTAGTAAATGGTTCTG
GGCAAACTGAATAGCCACATGCAAAAGAATGAAATTAGACCTTTATCTCTTACTTTATACACAAGTCAAC
TCAAAATAGACTACTTAAATGTAAGACTGAAAACTACACAACTATTAGAGAAGAAAAAAAACAGGGGGAA
AGCTTCATGATGTTGTTCTAGGCAAGGATTTTTTTGGATAAAACCTAAAAAGCACAGGCAACAAAAGCAA
ATATAGACAAATTGGATTATATTAAACTAAAAAGCTTCTGCACAGCAAGGCAAACAATCAATAGAGTGAA
GAGATAACCTACAGAATGGGAAAATAAATTTGTAAACTATGCATCTGATAATGGGTTAATATCTAAAACA
TACAAGGAACTCAAACAACTCAACAACGACAAAAAAAACAAATAATTCAAAAATGGACAAAAGACCTGAA
TAGATATTTCTCAAAAGAAGACAAAGAAATGCCAACAGATAAATGAAAATATGCTCAACATCACTAATCA
CCAGGGAAATGCAAATCAAACCTATCTTGAGGTATCTCCTCACCCTAGTTAAAATGGCTACCATCAAAAA
ATCAAAAGATATAAGTGTTTAAAGGATATGGAGAAAAGGGAATCCTTACACACTGATTGTGAGAATGTAA
ATTAGTACCACCATTATGGAAAGTAATATGGGGGTTCTTTAAAAAATTAAAAATAGAATTTTCATATGAT
CCAGCAGTCCCAATACTAAGTAAATATCCAGATGAAATGAAATCAGTATGTTGAAGAGATACAGTAAACA
TAGAATACTGTACTCCTATATGTTTATTGCTGCACTGTTCACAATAGCCAAGTTATCATATTAACCTAAA
TGATCGTAAATAAATAAATGCATAAAGAAAATGTACATAAGAAAAATTGAACGCCATTCAGCCATAAGAA
ATAATGAAATCCTGTTATTTGTGGCAACATGAATGAACCTGGAGGACATTATGTTAAGTGAAATCAGCCA
GGCACAGAAAGTCAAATATCTCACACATATGTGGAATCTAAAGGGTTGTCATAGAAGTAGATAGTAGAAT
AGTGGTTATCGGAGGATGGGAGGCTTGGAGGAGCGAGGTGTGGGGAAAGATTGGTCAACAGGTACAAAGC
TACAGTGAGATAGAAAGAATAAGTTCTGGTGTTCTATTGAATCGTTGGATGACTAGAGTTAACCATAATG
TGTTGTATATTTCAAAATAGCTAGAAGTTTTTAAATGGTCTCCTCACAAAGAAATGGTAAATGTTCAAAG
CTATGGATCTGCTAGTTACCCTATTTGATAATTATATAATGTATACATGTATCAAACTATCACATGTACC
CCATAAATATGTACAATTATTATGTGTCAATAATTTTTAAAATAAATGATGCCAACTTAATCCCTAGCTT
ACTTGGATTCAGGTTTCTGAGATATTATTTTCTTTATTCAGGGGCTATGGGTATATTTCCCCTTTTGCTT
GTTTAAGAACCTGCATTTTGAGCAAGGGGAAAAAACATCACTGACCTTTGATATCTCTCCCTCTCTTCTC
CCTACCCAAAAACCCTCTCTTTTATGACCTAGCATCCTGGTTTCTCAACAGAGTTTATGGAGAATTTTCA
GTGCTCTCCTAGTATAAGTTTTCAGGTTGACAGATTTCCTCAACCTGATAGACAGTTAAAAATACACTCC
AGAATCAAATCAAAATATTTTACAGTAAAACCTCTTATCAGAAAATAACCAAAGAGATGAGTTAAAACAA
ACGGTTTAGTCAAATATCTTAAACAGTTCTGAGGAAACAAATGGAAATAGAACAATAGCACTTCTCTGAA
GAGATAGTCAGTGTCAGAATCAAGAAAGACATGCTGTTATCTCAAAGGTACCCATGAGAATAATACTCTT
TTCACTGTTTCTAAGAATGCAGGCACTGAACTGATGGCTCTTCTCAGAGTCAGCCTTCTCCTGAAGTGCA
ATGAAGCCCTCCAAGCACACTCCAAACATTAACAACACACTACACAGGCTTTTTTACCACTGGCAGTGAA
GTGCTTCCTCATTAGAAGCCTTAAAGGAAGTATGAAATGCACCCATTTGTCAATAAATATTTATTACACC
TTACTATGCTCTAAGCACTGTGCTAAGAATTGCTGAGGGAAAAAAGATCATGTTCTCACACAGCTGCAAC
AATTTAATATCGGGATTAACTTGAAAGTTAAAATATAAGGGCTCAGGGTCTAGATATATAAGATTGTGAG
CTGATCCAAGGATGTATTAGTTTCTTACGGCTGTTGTAACAAATTAGCAAAACTCAGTGGCTTACAACAA
CAGAAATTTATTCTTTTGCCAGTTCTGGAGGCTAGAAGTCTGAAATCAAGGTCTGAGCAAGGCCATGATT
TCTCTAAAGGCTCTAGTAAGTATCCTGCCTTGCTGCTTTCTAACTTTGGAGGTTGTTGGCAATCCTTGCA
GCTTGCAGCTGCATCATCCCAATCTCTGGCCCGGTACTCACGTAGTTTTCTCCCTGTGTGTCTCGGTCTT
TTCTTCTTATAAGGACCCCAGTTATATTGGATTTGGACCCACACAATGACCTCATCTTCATTGTGTCTGC
AAAGACCATATTTCCAAATAAGATCACACTCAAACATGCCAGGGTTAGGACTTCAACATAACTTTTGGAG
GATTACAATTCAACCCACAACAAAGAATTTTGACTTAGAAGACTAGATGGTTATTGATACAATTAAGAAT
ACACTGATGGAGGAGAAAATTGAGGAGGTAGGGGAAATACAGAGTTTAATTTGGTGGCATCATAAATTTG
AACTACTTGCAGTACAGGCAGTGAACCTGAACAGGAGGTATATGGAAAGATAAATCTTAAGGAAAGAAGA
AAAGTCAGAGTTGTAGATAGGTTGGGATTTTGTATGCTAACTTTCATTAATCACGGGATTGTGTAGGGGT
CAGTTCCCTGGGAAACCAATCCTGAGAGAGAAATCTGAGATTTACTGAGAAGTGTTTTCAGGAACGAAAC
GTAACATCTGTAAAAAGTGAGGCATGCAAAATTATTGGGCAGAGAGAGAAGTTGAACTGTTAAAATTGCA
ACAGAGATTTCGGCTGATCCTATTGGCATCTCTGATGCTGTGACAACCCTCTGGAGTAGTTCCAAAACAA
AGCAAGAGGGTCAGGCCTTTCTTCTCCACACCATTCATTGGATGCAGGCTCTCCCTTTACCTTTCCAGGG
GAGAAGGGTATAACCTTGGGGGAAGCACCTCCCTTCTGCCACGATAAGGCCCAAAGAGGGATTCAGATGT
GAGCCTTCAGCATCCAACACTTTTGGCAGCTGGGGGAATGAGCAGCTTGCTGGAAAGCAGGATGTGGACA
GTGCCTTAGAGCATCTACTTCATGACAGAACCACAGACAGCAGCTGAGAACAGCGGATCAAACACATTTG
AGAGGTGGATGCAAAAGAAGAAAGCACGCACACACACACGGATAAAATGTATACAGTAGCCCCTTTACAC
ACATTATATAATTTTCGTCTTTGTGACCTCCTTAGGAAGTAAATATAATGCATCTATATTTGCAGATGAC
TGCGATTCTAAAAGATTAAGAAAAGATTTGGTTAGTCATGGACGTATCTGGGGGTCTTTTTAAATTTAAA
CCCCAAGCTGTAATCATTATACTACATGCCATAGAAAGGCAAGAAAATGAGTAGTAGTATAATTGTTTTC
TGCTATACCTCTGTAATGGTGTAATTCAGGGTGAAATTTTCTGTTGTAATGGTATTGTACATTTGCTGAT
TCATTGGGATTCTGATATTGTGCTTCTAATCCACCTTAATGCTGACTGATTAGGTTATAGTCTACCTTGG
TAATAATGTGTGATTTTTATATTTTAGGTGTTTAAATTTGTTTCTTCAAAACTATCTTATTCCCTTCTTT
ATTGAAAAGTAAAACTGATTTTACTTCTAGAATATATTTCTTTCTCAAGACTACATAACCCTTTCCTTTC
CATTTATTCAGTATTAAATGCATATTTTATACATTATGGTATTTCATGTGTATCTGTATATTTATTTTTG
CATATATTAATGTGTCTATTTTTTCATGCAAATTATTGATAAATATGACAAACTCATCTAGACCAAATTC
AAGTTCTTGTAGCTTCTGCCTGATACATTCATTCTTCTCTAGGTGAAAACCTTGCTGAGGTAACTATGAG
GTTCTTAGTGCTACTAAGACTTATCCAGCTCTCCCTGGCTTGTTGATGAAATTGGCAAGTGACTCAGAAA
TATCACTAAAGTCCAGATCATTGCCTTTAAAGCATCTCCTTTGTCATGACTTATTATGTTATTACAGAAA
AGTATTAAATCAATTTGGCATAATTTTTTCTACACATCACTTTGCTCTCTTTGAACCTTTGTGTTCTCTC
AGGACAATGTATTAAGTAGAATTGATTTATTATTATATTATCTTTCACATTAAGGCTATATTAGGGGCTA
GGGAGTACACTTACTGCCACACCAAGGTACACATGTTAAGATCTTAACTATTTGAATAAACCTATTTTAC
TTAAGGGTACATGGGTAATCCCTATTTTTTTGTGTGTGTGTTTTGTTATTTTTGGCTATTTCTGAGTCAG
GACTCTGTTTAACTGCATGCAACAGAGACTTAACTACATTGGCATAACTGGATAGAACATTCTGTTTTCA
TATTTTAAAACATCCACAGATCAGCAGTCCACGTGGCCTCCATGTCCTAGGCTCCATCTCTTCTCCCTTC
TGCCAATCTTCACATGTGTTTTTTCTTCTATATTAATGCCTATGATTAGAGGTGACTGTTCCACCTACAG
CTTTATATCCAATACCCAGGTAGAGAAAGAAATAGAACGAAGCAGAAGTAATGACTATCCCACTTGCCTG
AGGAAGGGCAAAACTTTCCCAGAGATCCTTATCCAACTTTGGCTTATGTCTTATTGGCAAGAACAGTGTG
ACATGGCCACCCCTAACAACAAGGTAGCCTGGCTGAGGCAGGACATTTATCTGGGCATATTGCTACCCTG
AATAAAATCTGGATTCTCTTGGTAAGATACCTTGGAAGGTATTAACAAGGTCAGGCATGCTTCTCCAATA
TTACTACTGCTACTAATTATTATTATTCCTTTATGAAAATGTCAAAAGAATTGAAGGATGGTAATTTTCT
ATTTTAAGAGATGCAGTTACTAGGCATAATTTGGAATAATATGCATCAATTGTAATGTTATACTTTGAAA
CATATTGTATAAAATTACCAAAAAACAAAAGTTAATTTTTACTTATTGAATTAGAAAATATATATGAAAA
TAAGAATATTTTAAGTTAAATAATAAAAAACTAAATATCCAACCATTTTTAATCTGCAAAAACTAGAAAT
TTCATGTGGCTCAACATAATACAAAGGCATAAGTAAAATATAAACTTCATTCTACCTGTATAATCTTATT
TCTTAATTACATAAATATTATGTTACTGAACACAGATATTTTATTCATTTCTTTTATTTGTGCATAGTAG
AGAACACATTAAAATTTTCTATGACTCTCTGCCCAGAATTATAATAGGCATAGAGTTTTTCTCTCCTAAA
TTACACTCATGAAATATTTTTGGCCCCCTATAAATATTGAGTCCCCCAAAAACCACACTAAAAACTTCAT
GTACTTCAAAGGTAAAGGGTGATTGATCATTGACAACAAAAAGTATACACTCTAATAGCTTGCACCTTAG
GTAAATATACAAAAAATTATAACACAGAACAAAGCAAAGCACAATCCGTAACAGGACACTAAACTAGTCC
ATTGAGGATGCATTGAGTAAGGGGCTGTATCTGGCTGACTTCACATGAAAGGAGGTGGCACTTAGACCAG
ACCTTACAGTATGGAGAAAATTTCACTGGGCAGATGTGGTACAGAAGGAAATCTCAGGCAGAGGAACAAG
CATTCATATTGTTCACCTGATTCACTCATTCATTCATCTATCCACCCATTCATCTATTCAGTGTTCAGTT
TACAAAGTCATTATCTTTGGAGATACTTAAATATGCTTATTATTATGAAATGTAGTCATTTTGTTGACCT
GATAAAAAGTTGTTTCAATATTTGCAGTTCCCAAATTAAATATTTAATGTCCTTTTACTATGTGGTGGTT
TTAATTACACTGTTATCAAGACTTTCTCTCTTTAGATTTTTAATCAAAGTTTTAGATTTACCTTCTGCTT
ATTACTACACTTTAGAAAATATAATAATCTTTTAAAAATGCATTTGACTTTGAAAGATTTAGGGATTAGT
TACAGTTATGCTGTTTCGTAAAATTGGCATTTGATTCTATATTTTATGCATAGATTTTTTTTAAAAGCAC
TCTTCTGTAGAATTGCACTTAGACCATTTGTTTTTTTCAGGTTGTTTCCAAAGAATTTGAAAAATCTTTC
CATCTTCATTGGATTTGTTTACGTGCCTAAAAACAATGAAATATTTGTCTGACATATTCAAATCAATGTG
TCTTATTCTTTATATTTTGATAAATTTGGAAACTTCACTTTTTTGCAATGCTGTTTAAAATGTCAAAATT
AATTAAATAAAAATAAGTCAATCAAGAAATGCTAATTTTAGAATATGCTTTTTAAAGCTGCATACTCTTA
CTGCCACCTCACCCCCTATACTGAGAATCACTTATATTTAAATGTTGCTTGCATTTAAGGAAAAATAGAA
TGCAAAACATTGCATTAGCACATTATAGTATTTAAATTCAATTTTTAAATAGTAGAGTAGAATTATGTAA
CTGTTTAATTGATTGTTTTTAAGTCTCTGTTGAATGACATTTAATCTTAGTACTGTGATTCAAATATATT
GCTTAACGTTGAGTATATAGCATGTACTTTCAAATATGTAACTAATTTCTACTTACAGGTATATGACTTT
AAACATTTTAAAACACAGTTGACTAAACTCTTTCATCATGCTGCATTTCTAAAATTCAGTACCTAACCTT
CTTCTCACAGGGGTAATATCAAACTGGATCAGTCAAGGATGCAGCTCTGTGCTTTTAACCAGGCAGAGAC
ATATGTCACCACTTTTACACAGGTTACCTAAAAAAATCATACATACGATATTTTTTCAATGTCTTGCATG
CTGTTCAGCTACAAAACCTTTCTGCCTGACCCACTGCAAGAAAGTAGAAACATATACAGTTGTTCAATAG
ATAAAGTGACATCACAGCATGTCACCAGGGCAATTTGATTATTTTTTTGTGATTCTCTTGATGACTTCTT
GATGTAACCTTCATGTGCTTAAGTGATTTCAAGTGCATGATCTGTAGATTTTTATTGAATTGATTATAAA
AGTAAAATGCAGAATGTAACAGTAGCCATTGAAGCTACATTTAATTTGAAAACCTCTATCAACTTCCCTT
CCTGATTTAATCTACATAAAAATATGGAGAGATGTATATCACATTAAATTGTCACATTTAAATTAAGTAG
ATCAAAACTCTAAAAAATTGTGGAAGAAGTGTTTCTGGCATAAATATAAAATCTCAGAGCAGGAAAAGAT
CATTGTAATTCTAGCTTTGTATTTAATAATTCTGGTTATGTATTTATGGATAAGCAAATTGAGACTCATG
CTTTCTTAGTACACATCTAGAAAGTTGGGGTCAAAGCCAAAGTACCCTGCTTCTAGTATAAACTTTGAAT
ACATTTTGAAATGGTTTTATGCACTGTTAGTTATGGGACTTTATTTTTTTAAATCAACTCAGTAATTAAC
ATTTGATTTTATTTGCTCCTATATAAAATGCATCAGTTAAAAGTTGATAGGCCTTGCCTATGAATGAATA
AGTGATAAGTGTTCAAAATGTTTAATTTTATTCCAGAAGTAATATTACTCATTGATAAGAATAAGTTTCT
GATTATATACATAATCAGATCAGTCTCTAGAAACTAAGAGGTTTAGATAGAGCATATTATAAATCCTATC
TATCAATAACACATAGAACACTTTTACTGAAATTAGATACAGATTAATATGTATTGCCTTATTGAATATT
TCTAAAATGTATTAAAATACATACTAAGATATATTTTAATTTTAGCATACTTTGTTGAATGACTGAACTT
TAATAGTTCTAGGATGGGGGCAGGAATCTCTGCCAATAAGAATCTTGACTCCTTTTTATAATCTAGTTCA
TATTTTGAATTTCTCTGTCATTGTTGAGATGTTTGTATTATTGTTATCTATTGTGCAATGATGAAAGCAA
AACTTTATCCTGAAATTGACACACCTTAGAGATAATATTGCTAACAGTTTCAAATGAAAAATTAGTTATT
TTAGTGTTCCAGTTTATTTGAAGATAATTTTAAAGGTGGCCTAAGTAAAAGATATATTTTTCTATTTCAA
ATTTGGTGGGGATTTAGAGGCAATATTCTCATAGAAAACTATTTTATTCCAGAATATGGTGAATTTTCTT
CAACTAGAGCCTAAATAAGGATGCATATACACCCAATATTATCTATATAGGTATAGATAAAACATATAAT
TTTTGAGAAAGCTATTTCTAGAAATATATATACATGCATAGACATATAAACATATGCATATATATGTTTT
GAATTCATAAATTCTTATTACTTTTTTACCAAGCTGTTAGAAAAGATGAGGTACATGTTAATGAATGGTC
ATCCTCTTAAGAAAAAAACTGAATATTCAGTTAAGAATGACTTGAAATATGGCACTTTAGCAAAAGCTGC
CTACATAAAGGATATAGTTATTTTATCCATGTAAAATTTCTATTTATATTTTCAATATTAGAAATCCACA
TGTTTAAAAAGCTAGTAATTATTCCATTCATTAAAATTCACTTTTCCTTGCCCTCCTTCATTGTGGGTAG
TATTAGTAGTTAACAGAAATGATTCAGATATCTAGGTAGCTTACTTTGTGTTTGTCATTCTTGTTTGTTT
CAAAATTATAGATAATATTTACGAAGCTTGAGTCTGCGTATCTACACTTTGCCTTTATGTTTGCTAAGGT
TCCAATCCTTCTTTACCTACTTGTTGCATTTTTTTAAGCTGGCATAAATAGCAAAATTGTTTAAATGTTT
GCTTCTAAAGACTTTAAGACACTCTGACTTGACCCACTGTATTCCAGCTGCTTTCTCTGCAAACAGTGTA
GCAGTTCTGTGACAGAGCACAGAGAGAGCCCTTGAATATCAATGGCCACTTTACACTCCTAGCTCTTTGC
TTCTCTTCTTTTCATTAGAAAGATGCTTCAAGATGATACTATAGGACCTGGCTGAGAAATCTGTAGCCAG
TCTAGCCTTCTTACATGGCCAACTGTTGTTGCTTTTATATGATTTCTTTATATTCATATCTTATCATTTA
GTCACGTAAGTTTTCATGTTATTGGATAGAAAGTGCCCTAGGTAGAAGATTCTGAGAAAAAGAAGAGGTC
TCCGTGAAATCACTGGTTTTATTCTGAATTTCTCTGGTGGTGGCTGAGATTTGACCTCATTATTTCACCA
ACAGAATACTATATATCATGTTGACCTTGTGATGGAACTGTGTGTTTCACGCTGAATAAGTAGCTATTCC
TCCCAGAGCCTACACAATATTTCTCTGCTAGCAATAGACAAAGAAAAATAGGAAAGGAAAAAAAATTGGT
TTTCTTGCCCTTTTTTTAAAAAAAGAGCTAAAGTTTTAGTTGCCCATAATTAGAATGTGAGCCCAGAATT
TATTGCAACTAGTTAATAAACTGACAATTTTCTGCTAAATTAGTGGAAGTAACATTAAAACTTAGACAAC
AGAAGAAAATACAGTTGAGTTTTCCACTGATCAATAATATCTGGAGTATTTTTTAATTATTTCCATTTTT
TTCCTGAAATTCCTGAGCTTCAGTGATTTTTTAACTTGAGGCATTACCTTTTAAAAAGAATCTTGAGAAA
TTTCATATCAAAAAAAAAAGATTGTAGAGACCTCAAGGGGCCCAGAAATCTAAATCATGTTAAGAAATGT
GATTACACTCAAGATGAAAAGCACTAATAGTAATATCAATAATATTAATGACCTCCAATTTTAAAAACTC
TATAAGAGGATAATGTTCACATTTCTCCAGAAGGAAAAAAATAAAGGCCCTGAATGATTGCAAGAGAGAA
GAAAGGTTATTTTTGTTCTACACCAGAAGAGAGTATTTAAACATCTAGTTGTCCTGTAACATAATGGGAA
ACTTGGAAAATATTGGACTCTTATGCCACATAGTAACTATGTGTGGATTTCTTCTCAAAGGATCCTTTGA
TCCTTTAATTGAAATTTACTATTTTACTCCTTAATGATTCAAGAACACAATTATAGATATTGACCGTAAA
ACTACACAATTAAATTAAAAGATGGATAATAGTAACTTGAAACTAATTTTAAAATGTCTTTCTTGCTTAT
TAACAGATAATGGAGCTTAGTTTACCACTGTCTGCCATAATTTTTCTTCTCTTTTATACCCATCTCCTCC
TTTTGATTTGCTTCTCTCAACTTTCTTACTGTCTTTTGTGAGTATTTTAAGGCTCAGGTGAAATAACCAT
TCTAGGCTCTGCTCCTTAAAGAATATCTTTTTTTTTGTTGTGTGAAGCAAACAGTTTATAGTATGTACTT
GAAATGTAACATTTTTCAGTTCTTTTCAGCATCAATCATTCTCTTTGAGTAAGGTTTTTGTTGAGTTCTG
TGTTTTGTACAGTGCTGCTGTGTTTGATAGTTTCTTCTTTTGAGCGTTAGATTATCAAGTCATTAGACAC
TCCTACCTCACACCTCTTGGAACAATAAATAACTAGCATTAACCATCATCCATGATAGCATGAAATAGGC
TTACATCGTTAATGTTTATCTTAGGAAATGCCAGTGATAAATTTGTAAGTGAACAACTTAGGACATGTTC
TAATATGATACATATATTTAACACAATTTCTACTCAAGCAGTATAGTAGAAAACACAGCTTCAAGGCAAT
CAAAGGGAAAACCATGAAGAATAATAAAGCTTTTAATTTGGACATATTTTCAGTAATTGAAAAGGAGCAT
CTTAAATAGCTACATAAGTTTACAATCAAGATAAGGATTGTTTCACAGCTATAGTGTTGTATGGATGCAC
ACTGAAAAATATGGAAAGATATGTCTCTCTCTCTCTCTCTCTCTCTCTCCTCTCTCTCTTTTTCTCTCTC
TCGATTACCTCAAGGGGGTTGAATCAAGGAGATAGTGGAGGGAACTTTTATTTTGCACTGTAAATACTAT
TATACATTTAAAATGTTTGAGTCAACTATATCACTTTCATATTTTTAAAACACTTTTAGAAGGCACAGTC
AATATCATATTATTCAATAAAAAGGCAGGAATGCTAATTATGGTACACTTCTGATTCCTAATACTAGTGA
GTCCACCTTCTTGATCCTATGCTTATATTTATATTTACTTTATTTATAACTAGACTGCCAATGATGAAAA
AAATTGATACTTCCCTTTTATTAGAAATCAAATCCATGTGAAAATTACAGAAGGTCCTATTTATAATAGC
AAAAGAAATTTCTTTCATACTAAAGCATTTATTTCACTTCTCTAATATAGTCATTCTTTCATTTCCAAAA
TTACATGGAAAACTAAACAACAGATGATTGAGTTTAAAGAAATGAAGATAATATAGAAATTAATCATATA
AAATAATACAAATAATTGATATGATCTTCCTTGTATAGTAAAAAACATATATTCAAAGTACTTAAATATG
GTGATGCCTACTGTGAGAATTCAATATATTTCACCGCAGGAAAAAAAAACTTTATTATATATATCAGCCT
TTCTATATAAAGGTATTCTCTTAGAAATCTGCTAAAGAAGCTTATACTGTAGCAAGTATAGATTTCAAAG
GCTGAATTGACTAGAACTGTCTATATTGCTAAAGCATGCTTATAAACATTTAATTTTATATACAGTATGG
AAATAGTTACTGTTTGAATACTTAATGCATTCCCAAGAAAACATTGAATGTCTAAGAATCTATTAGCCAT
AATAATTTGTCTGTAAATACAAATTAATTAATAATAGTTCATGTTAAATATTTTGATAATTTCTGTCCCA
TTGAACAATAATATTGAATAACTTTATGATGTAAAATTCTGTGCCAAGCTGAATTCAATTGACCATTAAA
AAAACATAAAACCTGCATATGAAAAAAAGTTTCAACAACAAGATACACTATATAAAATCACGTGAATTTT
TACTTGCTCATAATCTAATTTGGCATAAATCTTTGTTAATGCTCTCTTAAACAGTGAGAAAACCAATTAT
TGATGTTTGAAGATGCTTTCATGAATTTAGCTAAAGTCAGAATTCTGCAAATAAGAATCATTTTATTTAA
AAGTTGTTACCAGCTCAGAGAAAAACCATTATAACTATTTCCTATTGTTAATATTGTGTCTAATAGCAGG
GGGGTGCTTTGTATTCTTTCTATCAGCAATTCCTCTCACTGCTTTACCTCAAAACCCAGGCCAATAATTC
TTTTTTCAGAAATGAAGGGCCTGGTCTTAACTTCACAGAATAATAGGGCCTTGTCTTTCACTTCTCTGGT
GATAGTTCACGTGCAGCCGAGAGGTCAGGAATTGAAGCCATTAAAACTGTAGTCTTCTAAATATGGTACT
TTGGATTTTGTGGCAATTGGCACAAAATACATATTAGTAATTATAAGTTTATCCAACATCGTTTCCTGGA
TTAATCTGTATTTTAAAACTTATATATTTTAAAACTCATTCTATAGTTTATACTTTAAAGCCTATTCTAT
AGTTTTATAGAATATTCAGTTATAATTAATTTCTATTATCTTATTTTAAATTAAACTAATTTAGATGAAT
TCAATCAGTTTAAATAACATGTTTTTGAGTGAAATTTTGTACTGATACTTTCATAACTGAAACTGTTATC
AAACTCCTGGACCTGGTGGGAATAATGCCTCTAGGCCAACATAGATTGGAGGAGTTCTCTTAATAGGTTG
TTCATAATTATTTACTCCAGTTATAAGAAAGACATTGTCTGCTTACATCATTCCTTATGTACAATACTTT
ATCCATGCCATTTTTATGATGTTGTATACTAGGTGATAATAATAGCATTGAGAATTTAATGTGGTACCCA
TCAAGAATACTTCTAGCTTCCAAGTCAATCTCTAAAAGTCAAACCTTTAGATTGGGCTATCATATTAATA
CAAATTCATAGATTGAGAATCAATTTATCTGAAATAAGTATTTTATTATTTTTAGCACTTGCACTTGCTT
GAACTACTCTAAAAAAGATAAAGTTGTAACTTAATAAGAGAATTGTGTTGTAGTAACTAGTAGCTTTCAA
TATTCACATGTCATTTAGCAAATGAGAATGAAAAATTAACTCTTCCATTGATATGCTGCTTTACTCCTTT
ATAAAACAATATTTGGGAAATTTGACAGAGTTTTGCTACAGTTGCTGTATGTATTTTTTGTAAAGATTTC
TTCGTGTGTGTGTGTGTATGTGTAAACATCATGGTTACTCATAAAGCTCTGGGTGAGAAAAACCTATTTG
CGTTATGATCACTTATATGATCATAATGACAATGAATTCATTATTCCATGTGTAAGTCTGCCATGTTTAA
TTTTCTTCGATCTTTGTTTCAGAGTTTGCATGTGGTGCTTACTTTAGTAGCCACTTTCTTGACATTATAA
ATAATACTGTTTGATCTTTTCTACTATATACTAAAATGTTATATACAAAATGGGTGGTGTTGGTATGGTC
ATTGGCATTTTGATTTCAAAACCGTATTTGATTTTGTGGTCTTGATCAAAGAACTTCTATCTATATCATC
CTGCTACATATATTTAAATTCAAGAGATAATAAAAAATACATTTGGACTTTATCTTGCACTTATCTTTTT
AAAAAATTTTAACTATCAATAAGTTTTTTGCCAACTATTTGATCTGAGCTTTCAATGATCAGGCATTAGT
AATTTGACACTTGTATTATATTGCAAATAGGGTATAGGTTGTCCTCAAATAATGATTGTCAGTGTCCATA
AATTTATATTAAAAATTTAAAAAATAAGATGTGGTCAATACTTTCCAAAAGTGATAGTTAACATCAATAT
ATTACCTTATCTAATTTTGGCCACGTACTTGTTAGTATAATAAACATAATAAAGTTTATTATTTTACAGG
ATGGTGTAGATAGAAAGATCAAGTATAGATACATTTAAGGTTACTTGACCTCAATATGTGTTTATACATT
TTTTATGGCCATCATACTATTAATCTTTTTTCATAATACTTTGCATGAGTCTGATTATTAAATAAGGCTT
AAATATCATATTTGAATGAACTCTAAATGAACTACCTGGTGGGGTGGTGAATTCCTTTCTAGAGATTAAA
CCGGTCAAGCTCCTCAGAGTGCAGCACAGTTGATAACCCTTTGCCTGGAGAAGGAGAAGAAGCAGAGGCT
GAACCTATGAATTCCGATGAGCCAGAGGCCTGTTTCACAGATGGTAAGACAAAAATTGAGACCTTGGTTA
GCATTCCTTAATTAGTGTTCTGGGGTTTGTCTTAACGCCTAATACTTACCAGAGGTTGTCGGTAAGTCAG
AAGACTCCATTCCATGCTGATTCAGCTATAGCTGTATGATGATTAAGGGCAAGTCACTAGAGCTCTCTAA
GCTTGAGTTTCCACATCTATGGCACTTGACTAACAGTAATTTCCTATCACTGTATGTTAGACTGTAAGCA
TGCTACTACTTCTTATTTTAGAGCCTTAATTCGTATTCCCAGATCAATGGAACCACCAGAATACAAGTTA
ATGAAAGAGTCTGTACTCTGGTCCTTTTAATCAAAAATGGAATCTACTCACTAACTCTCAGAAAACTGTT
TGATACTGTTGACTACAGTCTCTCTCAAAATTCTTCCTTTCCTTAGTTTCAGAGGCCATAGTCTACCTTT
GGTCTTTTATTTGTCTTAGAGTTCTGAGTTCCTCTCTCTATTTCTTTAATGAGTATTTTTTCTGCACATC
CTGTAAATAATGATTTCTCCCCACATGTTCAATCTTCAGTTATCTTTTCTTATTCTGAAGCTCTGAGGTG
TTTCATGCATGCCTGTGGCTTGAAAAGCCATTTCTGTGCTATTGACTCTTAACATCAATGTCTTTACCTC
TAACCTCACTCTTGAGTCCCAGATCTGCTTACAATTCCCCATTGGTCATCACCATCTGTTGTTTGTAAAA
CATTCCAAGTTTAATAAGTTTTTTTTTTTTTGAAAAGCAAAAACAAAAACAAAACTTTAGTATGGTTTAC
AGCTTCCTCCAGTCTTTATTAGTAACTTCATGATCTACCTTACCCTCAAGCTAAAGACTTTGAGAGACCC
TTAAAGTTTTCCTTGCTAGTCACAAGTTCATCAGGAACTCTGGAGATAGGGCCCAAGCCCTTAAGAGGAT
TTGCATGCACACTAAAATGTGAGAATCCTTGGCCTCTAGGGTAAAGTCTGAAGTCCTTAACTTGACATGT
AAAGTTCTTTACATTTGGCCTTAGTCTCTTGTTTTGTAACAGTACCTCCTTCTACTCCTGTATGCTTTCT
GATATGCTGAATTTCTTACTACTTCTGGAATTTGTCAGGGATAATGCAGTATTGTCTGAACAAGTATTGT
ATCATCTAGGTAAAAATACAAGCCCAGAGTAGGGCTGCAATGTAATACAGTAATACAAAGTTCAAACTTT
GGAGCCTGATAAACATATGTTCTAATTTGCCGACTTATCTGCCTGTGAGAACTTGTGCACAATTTAAAAT
CTTGTTCTTCAGATATCTCATCTGTAAAATGGGGATATCAGCATCCACATCACAGCGATGCTAAGAGGAT
CAAATAAGATAATGATTATAAACTTACTAGCACAGTGCCTATCTTCCTTCTTCTACTTTTCTTATTCTTC
AAGGATTCTGTTAACAATCTTATACAGAAGTTATGATAGCACAGATGCTAATGACTTATCTAGTCATGGG
CTTACCCTCAGCATTAGTATTTTTTGAAAAATCATCGACTTGCTTTCAGTGTCCAAGATTTTCTCAACTG
GTTTCTTATAACATTCATGGGTTAAGCAAAATGATTGGAAAATAAAGACATCAGCAAACTACAAAGAGTG
CATGGATTTTCTTTAAATACACACCAAATATCATTTTATGTAAGAAGCAAAGGTGTACGACATCCAGTCA
ATATAAATTTTGTGCTTGCTGAAATTAGTGCTATAAGCAAATGCACAGTTTCCTTTAAAAGTGTCCATGT
TACAGAGATTTAAGAATTTCATCAACATGTATGGCCTGCTCCTCAAGAAATTTATTAGCAGTATATAGAC
TTAAATTTGTATTGATGCTACCAAGATTTTCCCTATTCATTTTATGAATGATCATGACTTAAAATTCTAG
GATAACTAGAAAGAAGTTGGGCTTATCAATCAACTTAAAGTTTCAAACCAAAAAATGACTTCAAAAAACT
TAAACTCTTACAGAAAGTATTAATACTATGCTATTTCAGACTGTAGGTGCTGTTATAATAAGATATAATA
CAAAGTTTTTTTAATCCTATAAACATCAATGAAAATACTTTTGTGAATACTCTGAGATTATTCATTGCTT
CCAAACTGAGGCCAGTATAGAAGAAATATTATGGAAACACATTTTCCAATGAACCTGGGCTTGGACCATT
GAAATTTACTGAGGGAGCATATATCTCTGAGTCACATCCCTTTTATGTGCTCCAGCAGAGACTTCATACA
GTCTCTGGATCTGTAGCCTAATAAATTCAGTGAAGCAGCCAATTTGCTTAGCCTTTCAACCGTGTATTCA
CATAATCATTCTTTATTCCAGTAAACAAATAATTATTAAATACCTGTGTGCCAGGTCCAGTTCCAGGCAC
TGGAGGGAGATGGAGCTGTGAACAAAACAATACCCCTTGCTGAAGAAACATATCAAGATGTAGCAGGTCA
GATAAGTGAAAAGTGCTATGGAGAAGGTATATGGAAAAGAGGAATACCAAGAGTGAGGGGGAAGAACTTA
CTAGCACATAAATATGCTATCAATGAATGATTGGTCAGAGAAAGCCTGCCTATAAAGGTGGCGTTTCTTC
CGTGATGTAAAGAAAATTGAAGGGGGCCGTGCAAATATATGGAGGGAAAGTATTTTAGGCAGAGTAATGA
AGGCACATGCCCCAAGGTGAGGATATGCATAACATTTGGGAAAAACCAAGAGGCCAAAATAGATGGAGAA
TAATGAAAGATGGGAAGAGGATTAAAAGTTAAGGTCAGAGGCTGGGTGAAGTGGCTCACACCTATAATTC
CAGTACTTTGGGAGGCCGAGGTGGGCGATCACTTGAGGCCAGGAGTTCAAGACCAGCCTGGCCAACGTAG
CGAAACCCCATTTTTACTGAAAATGAAAAAAAAATTAATTGATCTTGGTGATACATGCCTATAATCCCAG
CTACTTGTGAGGCTGAGGCACGAGAATTACTTGAATCCAGGAGGCAGAGGTTGCAGTGAGCCAAGATTGT
GCCACTGCACTCCAGCCTGGACGACAGAGGAAGACTCTGTCTCCAACAAACAAACAAATAAAACAAATGA
CATTATTTTTAAAAAGTGTAGATCAGAGATGTAGTAGAGGCCAGATTATAAGACATTTGAAGAACATTAT
AAGGATGTGATTTTAAAATCTCACAATGTGCAATCTTAAGAGTGGAAGGGCGAATAGCAATGGCAGAAGC
TGGAAGATTTCTTGGAGTTTCTGCATAAATCCAGGTGAGAGGTGATGGGAGCTTAGACTAGTATGGTAGA
GTTTTGTATAGTGAGGGTGTGACATGCTGAGTATATTTTGAAGTTAGAGCTGAAGGGATTTGTTGATGGA
TTGAACATTGGGTGCTTAAAAACTGGAAATGCATAGTTTCCACATGAAGACTGAGAAGCATGGGCTTGTC
AGAATGAAGAATCATGAACATGTTTACTTACAAGTTGGTAATTGGTCTACAAGAGCTGGGCTAGAATCAA
TGCCAGTTCAGAGCAATGTTTCACCTCCAGAGATCTCTGCTACCTGAAAGGTAACAGAAAAAATTAAATG
AAGTATGAAAAAGAATTAAATCCAAGTAGCATGTTTTTACTGAAGATAAATGTTAAAACGATTTTGTTGT
TCATAGTCTCACATTTGTTGATTCATTTCAGCTTCATAATTATCACTTTACTAATGTATTTCATTGATTG
CATCTGGTTCCTCTTATTCACCTTTTCTCTCCCTCTTATGCAAGGTTGTTCCTTAGCTCTGTGTCTATTA
CTTTGTATTTGAAGACTTCAGACTGCTTCCGTATATCTTGCCCTGTTCTTTACTATCCTTAGTTCTACAT
GACTTGGCAACCTATATAATTATGAACATGACATGACTCTTTGGGTCTATGTGGTTTTATATTGTGCTGC
TTTTAGATTATCAAATTAGTAAATTACATAATCAGGATTAGAGTTAGCGAAAAATAACCTTTTTCACAAG
GTCAAGAGATCGAGACCATCCTGGCTAACATGGTGAAATCCCGTTTCTACTAAAAATACAAAAATTAGCT
GAGCCTGGTGGTGAGCGCCTGTAGTCCCAGCTACTCAAGAGGCTGAGGCAGGAGAATCGCTTGAACCCAG
GAGGCAGAGGTTGCAGTGAGCTGAGATCCTGCTACTGCACTCCAGCCTGGCAACAAAGTGAGACTCTGTC
TCAAAAAATAATAATGGTAATAATAGTAATGTATTTTTCTGTTAACTTGGTAGGAGAAGCCAAATCATCT
TCCTTTCAAAATGTTTTGTATTCACCCCCTTCCTATAAAATAACTAATAATTAATTCTAACTTTAGTAAC
ACAATAACCAACTCCTTTGTGACTTTTATTTTTTCTTTCTTTTGGAAAGATAATGTTAACAAATATTTGT
TTTAGTTATATTTGATGTTAAGAAATGGTGAAAAAAGCTTGTTTTCATAATCAATTTTTAAATTAATTAT
ATTGATCATGCTGTTATGGTTTTTAATGAATAGTCTGTCTTTTGAAGTGGTCAGTGGTTTCTTTTTTCTT
TCCTTTCCTTTTTTTTTTTTTTTTTTTTTTGAGAGCTTTGGTTTAACATGATGAAACTCAGCATAGACTT
TATGTAATGCACCATGAGAGCACATTAGTGAAACCACCCAAAAGAAATAGCATTTACTCACTGGGACCCA
TTAAAATGTTTGAGAGATTCTCTGTTATGTTTACATTCAATTAATAGTCCTTTTAAAGAGAAGGTATGAT
AACATTGCTAACCAGCTTAGTTGGAAATCTCTTGGGATATACCTTTGCACAAAAACCAGACAGGCGCTCC
ATCACTCTTGCCTCAAAATAAGAGAAAGAAACTCTCTGAGGTGTAATAAACCCTGAGGGCAATATTTATG
AAACTTCATTTCAGATTGAAAGTTTACTAACTTTTCTAATTAGGATTTTTAGTGTTTTTATTTCCTCAAT
CATAAGAATAATGTTTCCCTTCAATTTTAAGATTGACAAAAGTAAATCCCAGAGAGCAAAATCTTCTTAA
CCAAAGGGATAATTTTCTTATCCAATAATTTGCCCCTGAGAAAGATAAATTCTTTCTTATATGTGATCAT
TGTTTTTAAGTTTAAGACAAATTCTAAAGCACTAAGTTAATATTAAAAAATTCATCTCATTTTTTTCGCT
CTCTCTGGTATTTTTATTTTCCCTGAAAAAGATTAACTTAGAGTTAATTTTTGACCCTACCCTTTTACTC
TGTTTTTATATCATTTTATCGAATGTTCTCTCTTAACTAAATGCATGTAGATAATAATTATTTAAGTATA
GGATGATACATAATTTCAGATCTGAGATCATGTTCACTGTAATTAACATTGTATAAATGAAGCTATGTGT
AGATTTTCCATTCACCTTTTTTTTTCTTTTTCATCCAATGTTTATGTAAAGTATAGAATTCACAGAGATG
TAACCTTTCAAATTATGACTAACTGCTTAGAAGATTATATAACTCTTTTTGGGGAATCAAGTCTTTTAAA
AATCAACTATAAATGACCTATTAAGCCAAGTAGAGGACTATGATGAGTACTATAAGCACAAAGAGCAAGC
ATTACAGACTCAGTTTGAGTCTTCTGGGAACTTAGAAACTTCCAGAGAGAAAAAACAGGAACACATAAAA
TATACATGATAACAAAAACACCAGAGACATAGGGGGAGAGGGAAACACCTTGTTTGCTGTATTTCTATAG
AGCTGGAAATTAAAGTGCTAGAAAATAACCTTATATCAACTGGATCAATGCACTAATTAGACTTCAGAAA
CATGAAAAGATTCATTACAAAACATTTTCTACCAGGTGCATCTTGTCAAGAGGATTCTAGTTTTGTTGTT
TTAGAACATACAACTTATGATTATCTGAAGTTGTTACTTGAAAACTCAATACTTGTTAATGAATTATATT
GTTTTACAGAAAAGGAGATCAATCTTTTAGAAATATCTAAAATTCAAACTTCTGAAACAGGTATCAAAAA
AGTGCTGCCCATTCTTACTTTCCCACCTCCTGCCTTCATCTAAAATGAGGTTTTTCATCTAAAATTTTAT
GAGGAAGAATTCACTGTAGTTACTTTTTAAAGAGTCTCTCTTTTCTTCAGAGTAATTCTAGTTGCTTTAA
TAAACTTTAAATTGATTAAAATTTTTCAATGTTTCAATCATTCTCTTTCTGGGCCTCTTATGTTCATAGC
TCTGTTTATCACTTCTTATAAAACTTTTTACTCTTTGGTTTCTCTTTTACTGCACCCTCTTGGTCCTTCT
TGTTCATCTCTCATTTCATCTACTTCAAAGTTTTCTCCTCTACCTGTCCTAGTGATGTGTAGGTAAATAT
TTTTATAAACTGGTTCTCCAAGGAAAATAAAAAATGTGTACTTATATCATTTACTATAAATTTTACTGAT
ATTAGGATATATGACACACAATTTACAAATAATAAAATATTACAACTTTTTATTGAACATTCTGTATATC
AAATTGTTTCTCAAAGAATGCTTTTTTTGAACTTTGCCAAACTTTTGCATGTATAGGCAACCTATTTTTG
CAGCTAATAAACAAGTATAGTTTTAGCATGAAGACTGCTTGATATTTATGTTAGTCAGTGAAATAAAAAA
GAAATGAAGAAGCTATATGTCAGAACTTTACTCATTCATCAGTGATGTAAGCAATTATTTGCAAATCAAA
AAACAGAATTTCAATAATGAAAGGATATTTTTTAATTTTTGTGCTATTCAGAGTAATGGCTACAAACACA
ATACACCTTGACTGTTTATCTGTTTTATTGGCATTTCCTCCATCACCTTCTTAATTCTACTCTAGAAAAT
CAAGAAGCTCTCATTTGTAGTGCTTGGAGATTTTTGTAATGTAAAGATTCCCACCATCACTGATTTCAAA
CTACTGATGTGAAATTGCTAAATGCAGAGTTAAGAAGAGATTCACAGTAGCTCAACAGTGTGACATACTT
CCACCATCCAGATAGAATAGGCCAGATAGAATAGACCAGCATCCAGAAAGAAGAGTTGCACAGTAGCCCA
ATAGTATAGCATATTACTACTGTCCAGAGAGAATAATCCAGAGAGAATAGACCACCGTCCAGAAAGAAGA
GATGCACAATAGCCCGACAGTATGCCATACTTCTGCTATTCAAACGGAATAGACAAATAAAATACAGTAA
AATAATTAGGAAGTGATATGTCTTAGTATTTATTACCTTTCCTTAAGGTAAGTTATAGAATTCTAAGTTT
ATATAGTCAAATATTTAATAATGGCTGCTTTTAACAACAGACAAGCAGGAATTTCTGACAGTTTAACAAC
AAGAATTGGTATGGTCTGCTTTCAGCACACCATTGAATTCTCTCCCTTAATATTGTTGTTTCAGAATGTT
TCGGAGTTGGCCCTTTTTCTGCTCACCCTAAATACTCTTTTTGTGTGACCTCAAAAACTGTTCTGACTTC
AGCAGATACATGTATGCTAATTATTACTAAGAATTCTTTTCTAGACAGGTTTTTCCCGTTTCAGGTTCCC
ACCCCCATTTTTAAAATCTATAGGATATCTGTATTTAAACAATTCACAGGCATCTCAAAATCATTACCTG
CCGAGTCAGCTCCTCCTCCTGTGGTTTTCAATTCACTGCTAACATCCACCTGGTAGTCCTGGGATCCTTC
CTTTCTCTTACATTTGATATCCAATCAATCACCAAATCCTGCTGATTTTATCTGCCAAATATGTCTTGAA
TCTGTTACAACTCTAGTTCCTAACTAATCTTTCCCTCCAGACAGGATACTGGACCAGAATCCAAATGGTT
TTCTGGGTATGAAACTTTCTTCACCCCTACTCCTCTCTATTCACCCTTCCAACTTATCCTCCATGTACCG
TGGAGACATCTATTTACATTGCAATTTGCGTATCTCGTTCCCTGCTTATAACCCTTTACTTACTCCTCAA
CCTCAATAGGATATAAGCCAAACTCCTTAAGGTAAAGGCCAATGCTTTCCATAATCTGACCAATTTTTCT
AAGCTCACCTCTCACCCTTGCCTCTTGTGTACATTCTGCTTGCATCACAATTCTAAACTGGTTTAAATTT
CTATGCATAACAATTTTTGCTGCCTGGAATTCCACCCAAACTCTTTTTTACTGATATTTTGATTCAACCC
AGGCTCCATCTCTTCTAGGAAGCCTCTACTGATCTCTCTTTCTCAAGAAATAAGCCAGGTGAGATTGATT
TCTTCCATGCTATCAAAATGAACCCTGACTATCTGTCACTTTAGTTGCCATATCTAGTTATAGTCATTTA
TTTCTGTTTTTATCTACCTAACCAAAACCTAACATAAATTCACTGAGGAAAGGAAATCATGTACAGAGGT
GAATACATAAAAGATACGTAACAAGGGTTTGATGGACAAAAGAATGCGTTAATGAATGAATAAACCTAAC
CATTTTTAAGTACAATTTTCAATTTTTTGTAGCAGTATATTTTGTACATAAAAATTTAAAGTCACATTTG
TTTTTAACTTAGAATACTTGAAACAGTTTCATCTGGATCTTCTAATCAAGATGAGTTTCTTTTGAACTTA
ATTTCAGGAGTTTCCACTAATTTTCTCAATATTGTTTAGAAAGGTGTATTTCAGAGGGTTTTTACCATAG
AGGAGTGGAAGTATCTAAAATTATAGACTAGAAAAATTTATCTGTCAGAAAAGAAGACCCTAAAATTCAT
GTCGTCTTCAGATTTTAGTTCATTCATTCATTCACACATAAAATGAAAGTGGTTTAAAGACTGTGGAGGA
GACTGAAGAGAAATTGGGGATTACAGAAGACAAGAATACATTGTAAGCTAAAACTAACTAACAACTATTT
AGAAAACATCATGCAAAGTTAGAAGATGAAGGAAGGCAGCATCCAAATTAAATCTATCTGAAATTGCAAG
TATAGGTGTTGAAATTATTTGGTTGAGGGAGTATCACAGAAAGCAGTTTGCATTAAATAATGTATTAAAA
ATTTTATTTATTCTGAATATCAAGATTTAACATGCATGTCTTTCTTGTCAGGTTGTGTACGGAGGTTCTC
ATGCTGCCAAGTTAACATAGAGTCAGGGAAAGGAAAAATCTGGTGGAACATCAGGAAAACCTGCTACAAG
ATTGTTGAACACAGTTGGTTTGAAAGCTTCATTGTCCTCATGATCCTGCTCAGCAGTGGTGCCCTGGTAA
ATGATCTGACACCTAAGTCAATATATTGATTAAGTCAATATTCTTTAAAATGAGCTAAAAGATAAAAATA
TCATGGCAAGTTGTTTTACCTAGAATTAATCCCTATGATTAGTGCCAATGACAAAATTTTACTTGAGCTC
AGTAAATAGTTACTGAGCACTCCTACATGCCAGGCATTATCCTTGCTGCCCTGAATGCTGTTGTCCAAAG
GACCACCCAGATGGCTATAATAGAAAAGAGAATTTTACTGGCAATATCAGTTTGCAAAACAGGGAGAGAG
TCTGTGGCATAAACTGAAAGCACTTTCCCTGTAGAACAAAGAGAGAAGGTTAGAGGTTTTATTAAAATGG
GAAAATGTTATATATTGCTCTTTGAGAAAGTTCATTGGCCCTAGTAAGGATCTGGGGAACTGGTAAGCTC
CAACTGGCGAGCAACAGTCGTTGGCACAATTAGTCCTAGAGTGTAACAGGTTTCCTCAGAAGCTATAGAT
AAAACTAATTTCAAGTTACAACAAGCAGCTTCAACAGTCAGTCTTGCAGAGAATTATAATTCTTGGAGTA
ATGTTTTGTACCCTGAGAGCTTTTCCCCCTGCCTTCTTGACTCTTAGTTGACTGTTAGTTGAGTAGGACA
AGAACTACCCAATTTGTATGATCAACTTTCACAATGCAAAGATAAATAAGATCCAGGTACTGTTTATTCT
TAAGGAGCTCAGAGCAATGGAGAAGAGAAGAATACAAATGAGTAACTGTAGAGCTTGGATGTGTTTGTAA
GCAAAGTGTAAATGTGACAAAGTTCTGTAGCAAGGAGAACAACTGCTTGCAATTTCAGAAACGTAAATGG
TGCTTTTCAGGGTCTAGAAAGACGAACAGGAATTTTCTAGTTAGGCAAGGCAGGCTGAGGGGAACAAGTA
TACAATCGTAAGGAGGCATAAAACAGCATCAGATATTCAGGAAATTAAAAACTGCTGAACTGCAAGTGTG
GTGGGAAGAAGAACAAGTACAGGTGGCCAGAGATACTATGAGAAAGCAAGGTCAGCTTGTGAAGCATCTT
GTGTGTTATAAAAAGGGAAAACAGATGGTAGTTTTAGGATAAAAGTAGGACAAGTTTCTCATTCTTGTAC
ACATACAAGCAGCTTCCATGAGAGTTGAAAGAGATCTAGTAGAATTACAAGCTACCTGAACCTTTTTCTT
AATTGTCATTTTTCCTGCTGACGCTGACAGAGATGAGCCATTAGAAGCCATTAGGAGCCCAGATCTAGCT
AGATATACCACTGGAAGTTTCATTATACTAAAAATAGAATACTCAAGAACTTTGTAACTTGCCTCATAGA
CAGTCTTCATGAGTTTGGACTTTGATTTTACTGTACTTATATGCTAATAGGTTGATCTGATACTGTTTCA
TGGATGCTGGCAGAAGATATGAGGTGCTTGGGTCAGAGATAAAGGACTTTGTTACTTATGTGTGGAAAGT
AGCATGAGTTTCAACATGTTTCTGTTGGTTCCCCCTTTACCCAGGTCTCATGGGGGCATTATATGAGCCT
GAATGAGTGTGTACACATATAGTGGATTGATGTCACAGCTGAGGAACACTGAGCTTAGGGAATGCATCCT
TTTATACCAAGCAACATAAGCAACCCTGCTCTTTGTCCCAGAGACCCTACCTCATGTCTCAAGGTTGCTC
ACTGCAAACACAACCCTGAGGAATGTCCTAGGTAAGGAGCAGGCAGAGCCTGGCATTCTTGGCACACTCC
CCAACAACCTGCAGGGACCCACTGTGGCTTTCCTTTCCTATCAAGTCTCAACTCTCAGAAACAGAAAATG
TGATCAGAAAAGCTCTATTTTCCTTACCTGGAGTCATCCAAGTAGAAATCATGATAGTGTCTACCATGCA
ATGTATTCCTAGTAACTAAGGAGGTTTATCCCATGCTGACTCACATATGTGACTGAGAATTTCAGAGAGG
AGATTACAAAAAAATTAAAGTTTGGAGTAATTCCTTGTTCAAAATGTCTAAAACAGAGGAAGACATTTGG
GGAGGGCATTCTCAAAAGTGTATTTTAAATAAATCATATGAGGAAGAAAAGGGAAAGATATTTCAAGAAG
CTGGTGTTTGTACTTAGACAGGTTGCCAATTAAACTAGATTTTTTAAAAGTATAGCAAACCCCCATGACA
CGTGTTTACCTATGTAACAAACCTTCACATGTACCCTCAAACCTAAAATAAAAGTTAAAAAGAAGTAATA
GCTCTCATTTAGCTTATACTTATTGTGTTTCAGAATCTTCTTTTAGATTTTCTCATTTAATTTTCAAACA
ACCCTGTGAGATTATGATATTTTGCTTATTTTACCAATGATGAAGCTGGGTACAGTTTTAAAAGTTGCTT
AATGTCCCAAAGCTAGTAAATGACAGAGCTATCACTGAATTGACATCTAAAATCAAAGTCACCATATCTT
TTCACTGTGTTGTGGCTCCTCAGAAAGAAGAGTATACACAAAACAGAAATAAATGAGTGGTAACTGAATT
TAGTAACAGTAAAGCGTTAAGTCCTAAATCAGATAGAGCTCACATAAGGTCATTCTTGCCTCCTTGCTCC
TACATCCACAAAATTGGTTCTGGAAGCTAGAACCCCTGTGGGCTGAATTCTAATTCCTGCTCTGGGACCA
GTCAGAGAGAGGTTCTCATCATCAAGACTTCCTGCGGCTCTGCTCTGACCCTGTACACCAGTTCTAGAAA
CAGGCTTCGTTTTCCTTGTGACTAAGCCCCTTCCTTGTCTTTGTATTTCAAGGACTGAGTCATTGCCATA
GTTTCGGTTTAGATGGCTTGCCTGATTCCTCCAGAATTTGGGGGTGGAGTGGAGGGGAGGAAAATCCTGA
GGGCTAGAAGCCTCCCTCTGCACTTCAATTCCATTGAATTTCAGAACCTCTGCCAACTGCTGGGACCTTC
TTTTTGCATCTTAGTAGGTCCATTCAGTCTTTCTGCCCCAACTTGTCTCTTTTCTTCAATATTGATACCA
ACAGTTTCCCAAAACTGCTAAGGATAATTTTAGAGTATTGAAAGCAGAACGTAGAATAAGAAGAATCTGA
ATGGCACAAGTTCATGACTGTAGACAGGTAATAAATATAATATTAACCACGAAGGATACAGGTCTTTGAC
TTTCATTTTATCCAACAGGGTTAATAGTGTTTCGATCTGAAAGAGAAAGTTCTAACACTTTAGACCTGTT
GAGATCAGCAATCATTTTCAGTGTCTCAATGAGCCTTTTTTTTTTTTTTTTTTTTGAGACAGAGTCTTGC
TCTGTTGCCCAGGCTGGAGTGCAGTGGCGTGATCTTGGCTCACTGCAACAATGAGCCCAATTTTTCAGTC
CTTTCAAATTCTTTGTCAAGGGACATGTGGCTGAAGTCTAAAGTCAAAGTCACCCTGTCCTTTCACTGTG
TTGTGGCTCCTCAGACAGAAGAGCATACACAAAATAGGAGTAAATGAGTGATAACCAAAATTAGTAACAG
TAAAGCGTAAGCCCTACATTAGATAGAGCTCACCTAATGTTGTCACATAATGTTGTGTGTCACTTACTAA
AATTTCTTGGAAAGCATAGGAAGCACCGCATTAATGGAGAGAATAGAACAGTGATTTTTAGAGCTGAAGC
TTTGAAGGATAATATATGGATGAATAGTCAAAGGAAAAAGCACCCTCTGTGTTCTATAGGCCTATTAAGC
TACTGCAGTCATTCATTTAGCAAATCTTTCCTTTCCTGCTGCTGTTATTAACATGGTACTAGGCACTGGG
ATGCACAAGCATTTGAGAGCTGAAAGACAGGCTCCACATCCTACTGGTGGGGAAAAAGGGGAGTGTAGGA
TGTGAAAAGTTTATAACAGTGGCTGGCCATGGGATCATGTCTTAAATTGCTAGGACTTATCCAATTTTAA
TGGAGAAAGAGGAGGAATCAGATGGAGGTGGAAATGATCAGGTGTCAGAGATCATGAGTCTTTTTGTGGA
TTATAAGTAATTTCATTCTTTTGCACTTTCTCTTGACCTCTGAAGACCAGCAGCAGCACTGGTGAGAAGG
CTAGAACTAACTGTTATATGATAACTGTAAACCAGAAAAAATGTCAGCTGGCCCATGTCAATATTATGTT
TCAGCAGTCTTAGCATTAGAATTGTATTTTTCCTGTTTTTTTTAAATGAATCATGAAGCTTAAGTTGTGC
ATGATTGAAACTTGAATATTATTTCCACAGGCTTTTGAAGATATTTATATTGAAAGGAAAAAGACCATTA
AGATTATCCTGGAGTATGCAGACAAGATCTTCACTTACATCTTCATTCTGGAAATGCTTCTAAAATGGAT
AGCATATGGTTATAAAACATATTTCACCAATGCCTGGTGTTGGCTGGATTTCCTAATTGTTGATGTAGGT
ACTTTTGAGTACATTTTAAAAGAGGATTTATTCTTACTGTGTGTTGTGAAAAAACCTTCACCACTATTGC
ATATTATCATATTTTAATTTTGTTAAGCTTCATATATTTTCAGGCTATAGGTTTATTCTTTTAGAAATAT
TAACACAGCCAGTGGAAATATATCTGTGAGGAATATTAGAACAGAAATCGTTCAACAAATGTAAATTATT
TCTGTGACATTGTAACATTTATAATTGAGTTTTTTATGTTTTCTGCAAAGGAGATGTGATTTTTTAAAAA
ATATGATTTGAATGTCATTCTTGTTAAGAATAAAACAAAATTTAGAAGTAGGAGATGATAAAAGGGCAGA
GACACAGATTAAAGAGAGGAAATGATGATTATTCATAATGTAAAATTGGAAGATAAGAGAGGATGAGGGG
CACATCAAGTTGGCTGATATGTGAGCTAAAATTTTTCTAAAAATCATTATACAAATACAAAGTTAACTTT
GTATTTGGGGAGAGAGAAGTCATGAAATTTTAAAATATCTATCTCAGACCCAATTATAGGAAAAACCAAA
ATGAGACACAATTGTCTCTATCTCAAATATGCGACTTGCCCTTCATCTCCAGAATGGCAGTGGGGCAGAG
GAGTAGGCATTATTATTAATGTAATAATTTTAGATAATAAATTCAAAGAACTGTTGAATACTTGGATGAT
GGAAAATACATAATAATTCTTTAGTTTACATATTATTTTAACGAAGTAGAAATATTTTTACAGGCTTCTG
ATTGAGATAGAGAAGAAATCTTAAAATAGTCGGTGAGATCAAGGACTCTCAAATAGAAACTAACGGTACA
ATACCCATTATTATCATTTCATTGAATGTACTCTAATTATTTTATATTTTAATAATATAACTTAATAATA
TATATGACATATTTTGTATTAATTAAAATGGCTTTCTTACATTATTTTATAACCTCAATCTCCCAGTCTT
ATTTTTCTCAGAATGATATGTTCTTTATCTTTTACTAATTCTTCTAGATTTTCAGAAACCTAACCTTTGT
GTTTAGGGAAGAAACATCCTATAATCAGTCATAGTTTTTAGAGATTTGTTTTCCATATGATGCCTGACAT
AATTAGAGGTCAACACGCAAATTTGTATATGCCAATAAGTGCTTTAAAATTTAACCAAGGGTTTGCATTA
TCGAGCTTTTAAGAAAAGTTGAATTCATACAGCATCATATGTGGATTAGAAAATCACAAGTAAGCATGAT
CATACATAGTAAAATCTGACTGCAAAATGTTACCAAATTAGTCATATTTACTCAAAAGTTCAATTTCTCA
ACTGTTATTCAAAGCTTCATAAACTTTTGGCTTCTGGCCAGGCGCGGTAGCTCACGCCTGTAATCCCAGC
ACTTTGAGAGTCCGAGGCAGGTGGATCACCTGAGGTCAGGAGTTTGAGACCAGCCTGGCCAACATGGTGA
AACCCCGTCTCTACTAAAAAATACAAAAAATTAGCCAGACATGGTGGCAGGTGCCTGTAATCCCAGCTAC
TCAGGAGGCTGAGGCAGAAGAATCGCTTGAACCCGGTAGGCAGAGGTTTCAGTGAGTCGAGATCGTGCCA
CTGCACTCCAGCTTGGGCGACAGGGTGAGACTCCATCTCAAAAAAAATAATAATAATTTTCGTTTTCTTA
ATCACACAACATATTAAATGTTAAAAACAATAACTTTTTTCTATAATATGTAATGTGAAAAAATTTTTAA
CATAGCTATATATTACACCTTATATGTGTTATCTTAGGTTTCCTCTATGAGAATTCTAACTTCGGATATA
GTAATGATTTCTTGCTATTTTTCAATTAATCATTCATATATCAAGTTATCTAGTTACTGCCTAAAAAATG
AAAAATGATAAAATGGGTATTTTTGTAATATAGTAGTTTTGAAGAAAGGCTGCCCCAAATAAGAAGAAAT
TATAGAAGGAATGTAGATTTTCTTTTCTCTTAAACTGTAAATATGACGGGGTCTCTCTCTTAAAAAATTA
TACAATATTTTAGATATTTTAAAATCTTTAAAATATTATCACAAATACACTCATTTTCATCATTCAGTGT
AAGAAATGAAGGTTTACAAGTAAAATTGAACTTATTTTAAATTAAGAGATATGGCTGTTTAATAATGGAA
ATTGTTTTATTTGAAAATTTAAAGTGATGATTCTCAGTGAGTATAACCTCCTGTTCAGGGGTATGTAATG
TGCAGAAATCAGGGAGATAAACAACTCCAATTTGAAATATTATTTAATATTTTGGAAACGAACAAATATA
ATGCAAAGCACAGAGAGTAAAGCTCCACACAATATTCTTGCTGACAGAGATGCAAATTTTGGGACACAGA
TTTGGGGGAGTGGCTGATTTTTTTTTTTTCTGAAAAGTATAGAAACTTATCTACAGCAGTTGTTCTTGAA
GGATGGTCTCAAAACTGAAAACGTTGGCACATGTAGGTGAGAACTCATAAGAGATGCAAATTCTGCCCCC
AACCCCAAGACATACTGAATCAGAAACTCTGGGGTTGGATGTGGCAAATCATTGTAACAAGCCCTACAGA
TGATTCTGTTGTATATTCAGATTTCAGAACTACTTATATTATTTGCTACTCAGAGAATGGACTTGTCTGA
TTTCTGCAGGCTTCTGGGTTATTTGAAACTTACTTGATTAAACAGTGTTCTTCAGTACAGGCGTATATTT
GTATATATACGTGTGTGTTTATAGAAAGATGTGAATATATATGCATATATATTCTCAGTTACAAACTATA
CCATAGTCTTAATAATATGCATATATTCCGTGTCATAAAACTCACTTAACAGTGGCTTGGAGAACAGACC
TATTAGTATTTTAGGGGCTGTATCAATTATAACAGACATGTCTTAGTCTACCTTTTATTGCTGTAACTGA
ATACCTGAGACTGGTTAAAGTATTCAATAAAGAAATTTATTTCTTAAAGTTCTGGAAGCTGGGAAGTCTA
AGGTGAAGGGTCCACATCTGGTGGGAACTTTCTTGCTGGTGGGCACACCAGAGAGTCCCAAGGCAGCTCA
TGGCATCACATGGCGAGGGGTGTTCACTGAGAGCCAAACTGGGTTTTATAATAAACCCACTCTTGTGATA
TCTAACTCACTCCCATGATAACCCATTAAGCCCTTACTCTATATTCCATTCATGAAGGCAGAACCTTCAT
GACCTCATCACCTTTTAAAGGCTCTGCCTCTTAATACTGTTACATTGGGAATTAAGTTCCAACATGAGTT
TCAAAAGGGACAAACATTCAAACCACAGCAAAACATTAAAAAAAAAATTTTCTGATTAAAGAGGGAGCAT
ACATATTTTCAAGTGTTCTTAAGTGGAAAATAATGGATGTGAATGACTGGGCAAATTTAAGAGTGGCATG
AGGAGTAATTTATTCTGCTAAATACGTAGCTTTAGGAATTTAAAAAAAAAAGCCATGTTACATTTAGTGA
GACATCTGGAAATACTACATGAGGATGAGCACTTTAAAACACTTCCCGCTTCATCAGTCAGTTTACCTGT
CTCTGATGGTAACAGCAATGAACTTTTTGTGAACAATGACTTTTTCTACGCCTGAAATAGATAAATATAT
AAAGTTGGCTCTCATCTGTTCCTATTCTTCCCTCGAAAAATTGAATCTCTCAGTCACTAACTTATTCGTA
ATGAAGAACTGCATTCCAGACATTTTTCTTAGACATCGTCTCCTGCTACACAGGATGTGAGGCAAATGAA
TATTTTCCATGGAACTAATTCAGAGAGTAAACATATTTATAGATCCAGATCTAAGGTCATGGAGGAGCTC
ATTGCTTTGTAACTAAAATGTGTATTGATTGTATTGCTGTTCATGTTCTTTCTTAAGATAATTTACGATA
AGAACTCAAATTTCTTTTGGATACTGGTTATGGTAATTGTGGCTGATCTATCTAACATGATGAAGCTCTT
ATTATATCTATATTGATTGTGAAGCCAATGAGATCAGCTACTCTCATTCTCTCTTCAGTACAATTGTGAT
TTTTCTCCCATGAAAAACTATTTGGTAGATCTTTGGTGCTAATCTTTAGAAATTGTAGGGCAGAAGCAAT
CTTGTTGAGAATTTGCCCCAACTCAAAATCATGAGTCTTCAAAATGTTTTTTAATAAGATTTATACTAAG
ACATAGATTTAGTTGAGTGAATACCACACTTTTAGGGCTTAGCTTCAGAATCACACACTTCATTTTGACC
TGAGTTTTATAATTAAAATGTTCACCATTGTTCTAATTAAATTTTTAGATTTAATAAGGAATTACAAAGC
AATTGATTACAAAAGTGTGAAAAGCATATGCATGATTTCTATTCAAATAGAATAAACAACTTACTTTGCT
TACATTATTTCTTCTTATTTTGGTTAACCTTCTTTTAAATCTATTTCCTTACAGAAACATCCATCTTACC
CTATTTTTAGGCATTGACTACACAACTCCGTATATACCATCTTATTGTTCCTGTTGAGTTGCTTTTAGTG
AGTTTCAGAATTGACTTTTTCCTTTTATGCTTCATCATTTTATTGACACAATTAATGAAAATGTTATTTT
TATAGGTTTCTTTGGTTACTTTAGTGGCAAACACTCTTGGCTACTCAGATCTTGGCCCCATTAAATCCCT
TCGGACACTGAGAGCTTTAAGACCTCTAAGAGCCTTATCTAGATTTGAAGGAATGAGGGTAAGAAAATAC
TAAACTTTATAATGTTCTTATTTTTTAATGGGGTTTAAAATATAAAAACAATGGGTATGTAGAAATTATG
AGTTGTTTCTGTTTACATAAATACATAGGCTGTTTATGTATGTGTGTGTACATATACATATATACATATG
CATATATGTATATATGCATATGTATACTATATAGTATGCATTTACATACTATATACAGTATGCATATATA
CTATATATAGTATGCATATATGGTATATATATGCATATTATATACACTAATAGTATATATATGCATATTA
CATATACTAATAGTATATATGCATATTATATATACTAATGGTATATATGCATACTAATATATAAATACTA
ATTATAGCATTAATTAAATAGTATATTACTAAAAATAGTATAATACTCATTAGTATATACTAGTATATTA
CTAAAAAAGTATAATACTAATTAGTATATACTATTATATATTAATATATACAGAATATGAAAGACTATAT
ATAAAATATGCATATATAGTATAGTATATATATTATTTCATTTACTGTACATATTAGTATATATATGCAT
ACTCTCTCTCTCTCTATATATATATATATATACACACACAGTATTTCAGTGCATATTGTTTAACAAGGGA
ATAAGTTAATCACAGTAATCCTGCCTTTATCTAATATTGTCAAACAATAGAATGATCCAAATGAGTGACT
TTTTTCATATTAAATGGAAATCTTCCCTGTGCTAGGAAGTCCAAGTGAAAGGGGAAACTATTGTTCCTAA
CATCATCTCTCATACATAAGTAATCAAGGACCTACTTATATCAGGTGTTCAAATAATATTTGTTTAAGAA
ACTACCTGGGTAAGGAGCATTAGAAAAGATAGGTAAGTGAGACAGCATCAGCCATCACATTTAATTTCAC
CTTTAAAATAACATTGCAAAGTAAGTGGAATTATAACTAAATTATATTTGAGAAAACTAAAGCTCAGAGA
GTTTACTTAATTTGCACTTAGGCTGCAGAGTGCCTGAAAAGATACTCAATTAGCAGAGACAAACAAGTTA
TAATGAATAACAACTACCATAACCTAGCTTTAAGGATTGTATTAAACCAGCAACTTTGTAACACACATGG
AGGACAACAACTCTATTTTCTGTAATTAGTCCATATCCTCATGTGTTGGTACTCCTCTAAAATTTCTTCT
CCAGATTTATACCTGCTATTTTATGCCTCTGTTCTGGAATGCTTCCCCTGCCCTGTCCCGACCCTAACCC
GTTTGTATAGCAGACAAACACCTATCTGTCCTTTAAGGCTCAATTTAAAGTTGCCTTGTCCGTCCACCAG
GAGACATAACTGGATATTCTCTCTCTGTTCAGACACCTAAAATCCTCATTATATTGTATTTTACTTATTT
GTTTACTTAACTGCCTCCCTACCTTACCTTCTCTGAGCAATTTAACACGGAGATTAGTCCAGCTTACTGA
TACTTTAAGTGTGGTTTACTGACCAGCTATTAGCATTATCTACAAACTTATTAAAAAGACGGACGGATCA
TGAGGTCAGGAGTTCGAGACCAGCCTGGCCAATATGGTGAAACCCCATCTCTACTAAAAATGCAAAAAAT
TACCCGGGCATAGTGGTGCTCACCTGTAGTCTCAGCTACTAGGGAGGCTGAGGCAGAAGAATCGCTTGAA
CCCGGGAGGCAGAGGTTGCAGTGAGCCGAGATCATGCAACTGCACTCCAGCCTGGGGAACAGAGCAAGAC
TCTGTATCAAAAAAAAAAAAAAAAAAAAAGCAAATTCTTGGATCTCACCCCAGACCTACTGAGTCAGAAA
CTCCAGGATGGGGATAAGAAACTTGGGTTTTAACCAGCCTCAAGTGAACATTAGAGATCTTTCAGCCTAC
CAAATCTTCACTGTTATCCCTACAAACCTAATTATGACTGCAATCTCTGCTATTTTAATTATCCAGTGTA
TAACGGTTTCCAAGTATAAAAGAAAGATCATGATTATACAAAAAAGTTCATAAGCACAATTCAAAAATAT
TCTATCTACAAGTTGAAAATTACAAGTTTCATTTTGTGCAGAAAGGTGGTATTAGAATTCATTGACATTA
ACTTAATTTTTTGAGCATTAATAGAATGTTACAATCTATCACAACTTTCCCCTACAAAACCACTTCCTTC
AGTGATTAAGCCAGCCCACTATTTAACTCTGTTTGCTCTTTGTTCTTTATCTGTATTACAGGTGAATTGG
ATGCAGGAGAATAGTTTAGTATATTTTGAGAGGCAAATGTCTATTTAGTTACATTTGTTAACCATGCAAA
AAGAGACATTTATTCATGCCGCAGGGCTATTTTGAAGGATAAATGAAATAATGAAATAATGCATTTGAGG
CATATTGTAGTCAGCTTGTTTGAGTCCTTAGGCTCTTTAGCTTTCCCAACTGGCTTCTCATCAGACCAAT
AGGTGCCTGTCTTTCTCAGAGACCCATGATTCCATCATTACTCACATTTTTTAGAAAAACATTTTAAACA
CCAACTATGTGTCAGGTGTTGTGATATAAAAAACACAGAGTACAGTGGTACATAGACCCCGGTCCCCAAA
GAGTGAGTAGGGTCAATGGTTACAAGAGAGACAGACAAGGGCACATAATGATACAGGAATATACAGTAGG
GAGTGGCTATCCGGGGAAGTCTGGGAAGACATCACAAGGAGGTGACATTTCAGACATTTCATGACTGAAG
AGTAAGCCCCAAATCATAGGCAGATAGGCATTCAAGGCAGTGATACCACATGGTAGGGAGGCACAGAATA
GATTGTGCAAGGTGTGCTTGGTCAGTGACTAGATGTATCAGAGTGCCTACGTGATAAACATAGTAGGGAG
GGCAGCAGGAAATGAGCATGAAAAGGTAGGCTCTATTAAGAGCTGGCTACTTTGAACTTCATTCTCTATT
CCTATTCCTCCTAATCTTTTTTGAGGTCACATAGACTGTAAGAATCTGATGAAAGTTAGAATTGTCTTTC
CGGAAAGACAGCACAACCATAATGTTTGTTATATAATTTCATGCCCCCAGGAAACCTCTACAGGTCCATG
AACTCCAAATAATCATTGCTCTGGACCAAAGGTCAACAAACCCCAGCCTGTTTTTGTAAAAATGTTCTTG
GAACACAGCCATACCAGTTTGTTTATGTATGACCCATGACTACCATGGCAGAGTTGAATAGTTGCAACGG
AGATATAACCCACAAAGCCTAAAAATATTTATAAAAATGTTTGCAAACCTTCTCCCTAGATGATCTGATT
AGTAGTCAGAGATCCTTAGTATTTCTCTATTTTAAAGCATCTTGAGAACCTGCTTAATACATTGCAGAAA
AGAAGGATCCCAGTGCTCCTTCCCTAACAGGTTAATCTTTAACACATTTGTACTTGAATGGGTGCTCAAG
CTGAAGTGTAAGATGACAGGTAGGTTTTGAAAGATTAAGTAAATAAGAAAATCTGGAGAGTGTTAAATGC
CACAGGAATTGCCCTGGATGAAAACTGGAGTGGCAAAGACAAACTGGTGATCAATAGCAGTACAAATGCT
GAAAAGCAGAAATAAAGAAAGGATTAAAGAAAAACTGTTCCTAATTTTTGTCTTAACTGAGGCCAGCACT
ATATATATTTAGTCAAAGGCTTACATATAAAGTATTTAGAGTAATAATAATAAATTTAAAACGATTGCTC
TTATTTCGTAGAACCAGCCAAATCTAAAGGATTTAAACCATTTAAAGATTCTCAATCCTTTTGTGGAGAA
TGTCTCAAAAAAAGAGAATAAGAAAAGAAAATAATTAAATTAAATTAAAATGATGTCCAGTCATAATTCC
ACCTACCTGATATACACCAATAGTTTCTCTATGGGGGAATCTTTAATGCTGGTAATAATAGGATCATGAA
CATTTAAATGTATATAAATATATATTTTTCATTTATGTTTATACATTTCTTTTTTTTCCAGAGTGTACAG
ATTTTACAAGTTTACCTTATCAATAACAGGTTTTGGTTTATTATTTTTATTTATTTTACATTTATTTAAG
CTATTATATTATCTTAGTTATTTTGTCTTATATTTTGCTAACAATTTTATTGTGTGATAATCATGTAATT
TGTAAGCCTGGTGATCACAGAGTTCATGTCTTATTTTGTCTTGCTTGGCACACTGCTGAGTGTAGAGTAG
GCATTTAATAAACATGAGTCGGCTGGTTGGTTTGATGTCTTAGATATTTAAGGGCGGCTTTTCTAATTTA
TTTATGTCTGCATGGCATTTCTTTCATGTTGCCTATTTAACATCTTACTAATCCTAATCATGCTTTTCTT
TCTTTTGAATACTAGGTCGTTGTGAATGCACTCATAGGAGCAATTCCTTCCATCATGAATGTGCTACTTG
TGTGTCTTATATTCTGGCTGATATTCAGCATCATGGGAGTAAATTTGTTTGCTGGCAAGTTCTATGAGTG
TATTAACACCACAGATGGGTCACGGTTTCCTGCAAGTCAAGTTCCAAATCGTTCCGAATGTTTTGCCCTT
ATGAATGTTAGTCAAAATGTGCGATGGAAAAACCTGAAAGTGAACTTTGATAATGTCGGACTTGGTTACC
TATCTCTGCTTCAAGTTGTAAGTGTCCCATTTCATGAGTGCTTGGTATTTTAATAGATATTGGACGAAGG
ATATAAGTTATTCTTTAAATAGTATATTAATTATAAATTGAAGATAAAATACTGTGTGGTTTTTAAAACA
GTGCCAGTCTTGGCCGGGCGCAGTGGCTCATGCCTGTAGTCTCTTCACTTTGGGAGGCCAAGGTGGGCAG
ATCATGAGGTCAGGAGTTCGAGACCAGTCTGGCCAACATAGTGAAACCCCATCTCTACTAAAAATACAAA
AAAATTAGCTGGGTGTGGTGGCAGGCGCCTGTAATCCTAGCTGCTTGGGAGGCTGAGGCAGGAGAATCAC
GTGAACCTGGGAGGCGGAGGTTGCAGTGGGCCAAGATCGTGCCACTGCACTCCAGCCTGGGCTACAGAGC
GAGACTCTTGTCTCAAAAAAAAAAAAAAAAAAAAAAAAAAGTGTTGGTCTTTCCTAGAACATGTTCCTAC
ATGAATAAGAAGCATGGGTTCTGTTGCATTTGAGTTATTAATTTATAATTGACTCTTTGGTGTCATGTGG
ACTCCCATCTTGCACCATTCAAATATTGCTTCAAAAATGAACGTTTTTGAACTTCCTGCTTCACACTATG
AAGGGATGAGAGGAACTTCCCACAGGCATGGAGAACCCTTTAAAAAATGGAGAAAAGATGTTTCCTGTCT
TATTCATAAAATTTTCCTTCACCTTTAAAAAATAAAAGGTTATGTGCGAAATTCACAGGCAACTGTAACA
ATTTAGAAATAATTAACTGGGACTAAGATTTAGAATGTTACTTGGGAATATGTCCTTTTTAAAAAAATCT
TTACATTAAATGGACATGTTGAATACAGCAAAACTTACTAGTATTACTTAATAATTGTGGTAACATTAAA
CAGACAAAACTCTGTTTATGGCTATTTTAGAATATGAGCTTAACATTCAAATTCTATTAATGTTATTCTT
AAAGGCAACTTTTAAGGGATGGACGATTATTATGTATGCAGCAGTGGATTCTGTTAATGTAAGTATTGAT
TATCTTAGCACTAAACTTTATTTTTAAAAGCTTCTTAGTTTATTTCAGTGATTTCCAAACTATAACTTCA
TGAAGCCCTTGGGACTCACATAGACAATGAAGAACATCCACCTGATAGTAGCCTAGTGGTTGTGAGTACA
GCAGTTGGAGATAGAGCCTGATTCAAATTCTAGCTGTACCATATCATCCAAGCTCTACAACTGACTTCGG
GCAAATTACCTTACCTTCTTGAGACTTAGTTTATTTGTCTAATCTATAATTTAATGAATCTATCATTTGT
CTAATAAATGTCTACAATCATCCAATGCCTACTCCATAGTGTTGTGAGGACCCAACAAGAAAATAGAAGC
AGGGCACTTAGTGCAGGCTGGCATTACTAAATGCTCAGTAAATATGCGCTTATCAAAATAAAAATAAAAT
AATTTTAATGTATAGCTTTTACTGTTTTTACAGTTTAAAACACCTTGGATGAGTTTATGTGAAATAAGTT
CCATGCTAAGAAACTTTAATGATTACTCCCTAGGTAATATTTTTACAAAATATAATAAAGATGTATTCGG
AATCAAAAGAAAATACATGGCAAAATGTGTATAAATATGATAATGTCTCATTTACAGTACTTTTGGAAAT
AACATATACATAGTATGTCACTTAGTTATTGAATTTTCCTACCAATATGAGCACAAATATGCATATGATT
AAGCCCTTTGTAGTTATTTGAAGATTATATTATTTATTACATAAGTATGTAATAAATTAAATAATAAGTA
TGTAATGAATACATGTTGAATAAATTGTTAAGAGTGATTATGTTAAACCTCAACAATGCTATGGCTTCTT
AAATCCTATGTTTACCTAGGATAGCACTGTTTGAATTACCTGGTCAAACCATGTGAAAAAGAAAATAATG
AATGTTACTGTCCATATAATGCTAACTTTTGTAAATTTTATAGGTAGACAAGCAGCCCAAATATGAATAT
AGCCTCTACATGTATATTTATTTTGTCGTCTTTATCATCTTTGGGTCATTCTTCACTTTGAACTTGTTCA
TTGGTGTCATCATAGATAATTTCAACCAACAGAAAAAGAAGATAAGTATTTCAAATATTTTTCATTGTAA
TGAAAGGGATGAACAATTATTTTCCAAAAAAGATAATGTAAAGTTTAATTTGATATCAATTCATGATTAA
TTTTATTAGAACTTCATTTATTTTTCAAATAATTGACTAGTATACAATTACAAATATGCTTAATCTTATT
CATTTTTGCACAGAAAACAATTTTCTAAATAATAGGTTCTGGCTAACTTATGATTTAGGTTAAACCCATA
GGTTTTGAGCAATATATCAACTATTCTGTTTTTTCCAATGTTTCCTATAGAGGATTGAATAATTAAGAAA
AATCAATACACTATTTATTCAAAACACATTTTATAAGTAGTTATTTATTTTATTTTGTGTAATATATATT
GGGTTGGAGCTTGGGGAGACCAACGTAAGATATGGTTCCTGAGCTCAAGGAGTTTATGTCATAGCAGAAT
AAATAACACAGGTAATGAAAATTAAGACTTGTTGAATACTGTAATTTGAGCAATCTTCTGAAATTTTTAA
TTAATTAATCTTCACATCAACTGTATTAGTTTCTTTGACCTACGAGAACAGGATAATACAAACCTGAGGG
CTTAAAACAAGAGAAATATATTCTCTCACACTTTTGGAGGTTAGAAGTCCAAATTCAAAGTGTCAGCAGG
TCCATGTTCTCTTTAAAGCCTCTAAGAAAATATATTTTATTGCCTCTTCCAGCTTCTGGTAGCCCAGGTG
TTTCTTGGTTTATGGCAACAGAACTCCAATCTCTCCCATTATAGTGACATGCCATTTTCCCTCTGTGTGT
CTTTCTCCTCTACTAGTAAGGATGCCAGTCACACTGGATTAAGAGCCCACTCCACTCCAGTATGACTTCA
TCTTAATTACATCTGCAATGACATTATTTTCAAATAAGGTCACATTCTGAAGTGCTAAGGGTTAGGACTT
ATATGTACCTTCTTAGGGCACACAATCCAACCCATAACATCAATCACAAAAGTATTATTACAGAGTCTCT
TTCACAAATGAGGAAAGGCACACAGAGGTTAAGTGACTTACCCAAGGTCATATATCGAGTATGTGATGAA
GGTTAGATAGAGTCAGTAGTAAATTCGATGTATCTTGTACTCTATTAAACTCTCTACTATGCTCTATCCC
CAAATACTATAATCATGATAAATACAATAAGAAAAATTAACAAGCACAAAAAATATACATGACAGTCAGA
GTAAAAATAGATGCTGCTATAGAATGGTTTATGAAATACGTGACAATTGAGAAGGCTTTGGAAGGTGGGA
AAGAAGACATGTAGCAACGGAGAGGCAAAAGAATGGCTAACCCACATGGAGTGAAACAGAAATGAATGAG
CAAATGAATTGTGTTGATAAAAGAGGCAGCAATTCAGGGGGGATAGCAATGCTTTCGGTTTTGAAGAAAT
TTAAGATTTGGAGAAATAGTAAAAGATGAGGTTTTAACCTGCCTGAGAGTAGATATAAAAAGCCGTATTA
GACCTTTTTTAATAGCAGCTGGTGTTTCATTCATATATGTTGAGTAGGGAAGAATATGGAAGCAGGAGAT
TGATGTTTATTAAGACTCCATGATATGTCAGATCCTGTGTAAAATACTTCTAACATAATTGTAGTCAAAT
TTTACAACAAATCTAAAAGACATAGTATCCTCATTTAACCTCTCAGAAAAATTATCTTATTTGCACATGG
CCTGACAGCTAAAAAGTAGTTTCATTATGGATCTAGCTCCAGAATTGCTTTTTCTAGAGACTGAGCTTTA
CAAAAAATTAATCTAGCAGTGATGCACAAAATGACTAGGGAAGGACCAAAGACTATTTCAGACCCTGCTT
GACCTGCCACCTGGACCACACTAAGTCTCCAAATTAGTCCTGTGTGTCAATGGAGAATAGGAAATATGTT
CCATTAAAACATTTTAGAGGGTAGAATCTACAGTATTGCTTGTGATTTGCAAGAGATAAAGGAGTCTAAT
TCTGGAGTTTCTTGTCAACTAACTGAGATACTCTGGTTGTGGTACCACCTATACAAAAAGAGATCTTTGG
AGACTAAATGGTAGAATTACAAAAGAGAATAATAATTTTGGGTACTGGCTTAGCATCTAAGAAGACAGAT
GTACATGTACCAAAATGTCATTAGTGTGCATGATTGACTATTTGCAAGTAGACAAAAATTTAGTAAATAT
TGCATTATTTATCCTGACATTTACAAATACAGATGTTTATTTTTAAAGATTTTTAAATTTCATGTTCCAT
TTTACATTTATAAAGTGTACACTGATTTTCAATTTATTTTTCTCAACTTAAGCATTATATATACATACAT
GTAATATATAAGCACACACAAGTAATTCCTCAGTCTTCCCTGCATAGGATAAATTAAGCGTGTCTAGAAA
GAGCAGTATGAGCATAAATAAAACAGCCAACATCCAATTGACTTTAATCTTCCTGCTTGTTAATGTTATT
CTTACAACATTTCTACTGCAGGAAGTAAAATAGGATGGGTTGATACTATCACCACCAAAGTCAGGAAACA
GTTACTGATTTAGAAGCATCAAAAACTCATATCCCCTATCATTCTTCCAGTATTTCATCTTGCAATCTAA
GTATAGCTTTTATTTTCTGACCTTTTCTGAATGATTTAGAATTTCTTTCCACTCTTCCCTGAAAAAACAT
GACCAACACTTCAATTAGTTAACACATGCTATGTAGAAGTACGTAGTAGATGCTTTGTGATGAACTCATT
TATTCCAAAGAAAAAAAACAAATGAAAGTGGTATCCTTTTCTTTGCCTTTCCTAATTTTAATTATTGTGG
TAAGGAAACTTAATATGAGATCTACCTGCTTAACAAAATTTTTAAGTATACTATATATATTGTTAACTAT
AGTTACGAAGTACTACAGGAAGTCCCTAGAACTTATTCATCTTGCATAATTGAAACTTTATTCCCATTGA
ATAGCAATTCTCCATTCTCCCATCCTTTCATGCCCTGGCAATCACCATTCTTCTCTCTGTTTCCATGAGT
TTGACCATTTTAGTTACTTCACCGAAGTGGAATTATGCAATATTTTTTCTGTTACTTCACTCAGCATAAA
TTTATCCAGGTTCATTCAAGTTGCTGCATATGGCAGGATTTCCTTCTTTATAAGGCTGAATAGTGTGTGT
TTGTGTGTGTGTGTGTACGTGCACACGCACATGCAGGTATACAAATACCACATTTTCTATCCCATCATTC
ATCAATAGACATTTAGGTTGTTTTCATATTTGGATACAGTAAATAGTGCTGAAGTGAACATGGAAGTAAG
GACTCTGAGATCCTGAGTTCAAATTTTTAAAATATGCTCAGATGTGGCATTGCTAGATCAAATGGTAGTT
ATATTTTTAATTTTTTCAGGAACTTTCATACTGTTTTCTATGGCAGCTACATCATTTTACATTCCAACAA
TGTACAAAGGTTCTAATTTCTCCATATCCTTGCCAACACCTATTATCTTTTGATTTGGGTGGTTTTTGTT
TTTGTTTTGGTAATAGCCATCCTAGCAGGTATGAAATGATATCTCTTTACGGTTTTTTGTTGCTGTTTTT
TTTTTTTTTTTTTTTTTTTTTTGTTGTTTTTTTTTTTGAGACGGAGTTTCGCTCTGTCGCCCAGGCTGGA
GTGCAGTGGCGCGATCTCGACTCACTGCAAGCTCCGCCTCCCGGGTTCACGCCATTCTCCTGCCTCAGCC
TCCCGTGTAGCTGGGACTACAGGCGTGCGCCACCATGCCCGGCTAATTTTTGTATTTTTAGTAGAGACGG
GGTTTCACCCTGTTAGCCAGGATGGTCTCGATCTCCTGACCTCGTGATCCGCCCGTCTCGGCCTCCCAAA
GTGCTGGGATTACAGGCGTGAGCCACCACGCCCGGCCTTGTTGCTGTTTGTTTGTTTGTTTTTGAAGGAG
TCTCACTCTGTCACCAGGCTGGAGTGCAGTGGCGCGATCTCAGCTCACTGCAACCTCCGACTCCCTGGTT
CAAGTGATTCTCCTGCCTCAGCTTCCCAAGTAGCTGGGATTACAGGCATGTGCCACCATGCCCAGCTAAT
TTTTGTATTTTCAGTAGAGATGGAGTTTCACCATTTTGGCCAGGATGGTCTTGATCTCCTGACCTCATGA
TCCACCCACCTTGGCCTCCCAAAGTGCTGGGATAACAGGCATGAGCCACTGCACCTGGCCCTCATTACAG
TTTTTATTTGAATTTTCTGATGATTAGTGATACTGAGCATCTTTTAATATACATGTTGGTCATTTGCATG
CCTTCTTAAGTTGCAGGAGTTCCCTATATATTTTGGATATTAACCATTTATTAGATATATAGTTGGAAAT
ATTTTCTCCCATTCTATAGGTTGCTTTTTCACCCTCTTGAATGCTTCCTTTTTTTCCCAAAGCTTTTTAG
TTTGATATGGTCTCAATTGTCTATTTTTGCTCTTGTTTGAATTTTGGAGTCATTGCCAAGAAATCATTGT
CAAGAACAGTATCATGAAGCTTTCCTGCTATGTTTTCTTCTACAAGTTTTACAGTTTTCAGGTCTTAGGT
TTAAATGTTTAATCTATTTTGAGTTTATTTTTGTGTATGGTATAATATAAGAATTCAATTTTATTTTTTT
ATATGTGAATATCCAGTTTTCCCAACATTATTTGTTGAGTGGCCTCTTTTCTATTGTGTATTCTTGGCAC
CCCTGTTGAATATGAGTTAACTATTTATGCATGGGTTTATTTCTGGGCTCTCTATCCTGTTTCATTGGCC
TATATATTTGTTTTATGCCAGGACCATACTATTTTAATTGTTGTAGCTTTGTTATACATTTTGAAATGAG
GACCTGTGGGGACTGGGTGCAATGGCTCATGCTTATAATCCCAGCAACTCGGGAGGCTAAGGCTGGAGGA
TCACTTGTGGCCAAGAGTTTGAGACCAGCCTGGGCAAGATAGACTCCTTCTTTACGAAGATATTTGAAAA
GTAGCCAAGCATGATGGCACACACCTATAGTCCTAGCTACTTAGGAGCCTGAGGTGGGAGGATCACTGAA
GCCCAAGAGTATGAGGCTGCAGTGAGCTATGATCATGCCACTGCACTCTGGCCTGGGCAACAGGGTGAGA
TCTTCTCTCTCAATAAATAAATAAATAAGAAATGAGAAAATGTGATTCTTCCATTTTGTTCTTTTTTCTT
AAGATTGCTTTGGCTATTCAGAGTCTTTTGTGGTTCCATATGAATTTTAGGATCTTTTTTTATTTTAGTA
AATAATGCTATTGGGATTTTGATATGTACTGTATTGAATTCACAGATCACTTAGGGTAGTATGGGCATTT
TAACAATATTAAGTCATTTAATTCGTGAATACTGGGTCTTTCCATTTATTTGTGTCTTCTTTAATTTCCT
TCATGAACATTTTGTAGTTTTCAGTGCATACATTTTCAATGCCTTGGTTAAGCATATCCTAAGTATTTTA
TTTCTTTTGATGTTATCATAAATGGGATTTCTTTCCTAATTTTCTCTTCAGATTGTTTATTGTTAGTGTA
TAGAAACGCAGGTGATTTTTGTATGTTGATTTTATATCCTTCAACATCTAGTAAATTTGTTTATTAGTTC
TAACAGATTTTCCTGATGTGTGGAGTCTTTAGGGTACTCTCCATATAAAATAATGTCATCTGTAAACACT
TTTTTCTTTCAGATTGGGATGTCTTTTACTTGTTTTTCTTGCCTAATTTCTCTGGCTAGGACTTCTAGTG
CTGTGTTAAATACAAGTGGCAAGAGTAGCCATATTTGTCTTATTCCTGATCTTACAGGAAAGTTTTTCAG
TTTTTCACCATTGAATACGATGTTAGCTGTGGGATTTTCAAGTAGTCTTTGTTATCATGAAGTATATTTC
CTCTGTTCTCATCCATTTGTGCTACTATAACAGAATACCACAGACTGGGTAATTTATAATGAACATAAAC
CTTATGGGATCACAGTACTGGAGGATGGAAAGTTCATGACTGAGGGGCTGTCTGGTGAGGTCTTATTTGT
TGTGTCATAACATGCTGTAAGGTATCATGTTATGATACACATGGCACAAAGAGAGAATAAGTGAGAAAAG
GAGGCAGAACCTGCCTGTTTATAATGAACTCAATCCTGAGATAACAGCATTAATGCATTTAAAAGGGTAA
AATTCTCATAACTTAAACACCTCTTAAAGGTCTCACCTCCCAATTCTGTCACAATTGCAATTAAATTGCA
ACATGAGCTTTGGAGGGGACAAACCTTCAAGTTATAGCAACTTCTATACCTAGCTTTTGTTTTCTTTTTT
TAGTTTTTACCATGAAAGGTTGTTGAAATTTGTCAAATGCTTCTTCTGAATCTCTTCTGATGATCATGTG
ATTTTTATCCTTCTCAGATCATGTGGTGTATCACATTATTGGTCTGCATATGTTGAACCATCCTTGCATC
CTAGGGACAACTCCCACTTGGTCGTAGTACATGATCATTTAATGGGCTGTTGCATTCAGTTTGCTAATAT
TTTGATGAAGTTATTTGCATCTATGTTCATCAGGGATCTTGTTCTGTAGTATGTTTTTTTCTGTTTAAAT
ATTTGTTCATTTTTAATTGACAAAAATTGTGCTTATTTATGGGGTACAAAACAATGTTTTGATCTACATA
AACATTGTAGAAATTTTCAGTCACGCTAATTAACATAACCATCACTTCACCCACTTATCATTTTTTTGTG
GTAAGAAAGTTAAAAAGCTATTCTTTTAGCAATTTTGAAATATACATTTATTTTTTTCATTTCCTATTGT
GTGACTTTTTTGACTTTTATTTTAAGTTCAGGGGTACATGTACAGGATGTGCAGTCTTGTTACATAGGTA
AACATGTCATAGGGGTTTGTTGTACAGATTATTTCATCCCTGAGGTATTAATCCCAGTATGCATTGGTTA
TTTTTCCTGACCCTCTCCTTCCTCCCACCCTTCACCCTCCCATAGGCCCCAGTGTGTGTTGTTACCCTTT
ATGTGTCCATGTGTTCTCATCATTTAGCTCCCACTTATAAGTGAGAACATGTGGTATTTGGTTTTCTGTT
CCTGCCTTAGTTTACTAATGAAAATGGCCTCCAGCTCCATTCATGTCCCTGCAAAGGATATGATCTCGTT
TTTTTTTGAATGGCTGCATAGTATTATATGGTATATATTTACCACATTATCTTTATCCAGTCTATCACTG
ATGGTCATTTGAGTTGATTCTATGTCTTTGCCATTGTGAATAGTGCTCCAATAAACATATGTGTGCATGT
GTCTTTATAATAGAAAAATGTATTTCCCTTTAGGTATATACCCAGTAATGGGATTGCTGGGTTGAATGGC
ATTTCTGTCTTTAGGTCTTTGAGGAATTGCCACACAGTCTTCCATTATGGTTGAATTAATTTACACTCCC
ATCAACAGTGTGTAGGCATTCTTTTTTTCTCCACACCCTCGCCAGCATCTGTTATTTTTTGACTTTTTCA
TAATAGCCATTCTGACTGGTGTGAGATGGTATCTCATTGTGGTTTTGATTTGCATTTCTCTAATGGTCAG
TGACATTGAGCTTTTTTTCATATGCTTGTTTGTTGCATGTGTGTCTTCTTTTCAGAAGTGTCTGTTCATA
TCCTTTGCCCACTTTTTAATGGGGTTGTTTATTTCTTGTAAACTCATTTAAGTTCCTTATAGATTCTGGA
TATTAGACCTTTGTCAGATGCATAGTCTGCAAAAATTTTCTCCCATTCTGTAGACTGTCTGCTTACTCTG
TTGATAGTTTCTTTTGCTGTGCAGAAGCTCTTTAGTTTAATTAGATCCCATTTGTTAATTTTTGTTTTTG
TTGCAGTTGCTTTTGGTGTCTTCATCATGAAATCTTTGCCCATGCCTATGTCCTGAATGGTATTGCCTAG
GCTGTCTTCCAGGGTTTTTATAGTTTTAGATTTTACATTTAAGTCTTTAATCCATCTTAAGATTTATTCC
TCTACTCTAAATGAAAAATGTACATCCTTTGATCAAGATTTATACTTTCTCCACATCGTCTTCCCCCAGT
CCTATGGCAACCACCTTTATATGCTGTTTCTGTAATGTTGACTTCTCACTTTTTTATTTTTTATTTATTT
ATTTTTTTATTTTACTTTAAGTTCTAGGGTACATGTGCACAACGTGCAGGTTTGTTACATATGTATACAT
GTGCCATGTTGGTGTGCGGCACCCATTAACTCGTCATTTACATTAGGTATATCTCCTAATGCTATCCCTC
CCCCCTCCCCCCACCCCATTTTACATGTAAGTGAGATCATGTGGTATTTGTCTTTCTGTACCTGGCTAAT
TTTGCTTAGCATAACGTCCTTTAGTTTCATTCATGTTGGGATGAATGACAAAATTTGTTCTTTTAAAGTT
TTGTATAGTAGTGCACTGTGCATACCTATCACATTTTCTTTATTCATTCACCTGTTGATGGGTACAGGTT
GCATGTATGGGTTAGTGCAAAAGTAATTGCGGTTTTGGCCATTACTTTTAATTGCAGCAAAAGTAATGGT
AAAAACTGCAATTACATTTGCACCAACCTAATATGTCTTGGTTCTTGTAAATAATGCTGAAATGAACATG
GGAGTGCAGGTATACCTTTGACAGAACGATTTTAATTCCTCTAAAGGAATTAAATATATACCACTATATA
CCTAGAAGTGGAATTGCTGAATTATATGGTAATTGTATTTGTTTTGGGAACTTCCATGCTATTTTTCTCA
AACAGCTGTACTAATTTACATTTTCACTAACAATGTATAAGGGTTCCTATTTCTCCACATCCTCATCAAC
ACTTGTTATCATTCATCTTTTTGATAACAGCCATTCTAGGAGGTGTGAGGTAATATTTCATTGGGATTTT
AATTTGCATTTTTCTGATGATTAGAGATGTTGAGCATTTTTTAATATATCTATTGGCCATTCATATCTCT
TCTTTTGAGAAATGTCTGTTCAGATTATTTGCCCATTCTACAATTGGTTTATTGATTTTCTAGGTGTTGA
GTTGCTTGAGCTTTTTATGTATAATGTTGGATATTAGCCCTTTATCAGATGTATGGTTTGCAGATATTTT
ATTTCAATCCATGATCTCTCTCTCCACTCTGTTATTGCTTCGTTTGCTGTAAAGAACATTTTTAGTTTGA
TCTAATCCAACTTGTCTCTTATTGTCTTTTGTTACCTGTGCTTTTAGAGTCCTAGCCAAGAAATCCTTGC
CCAGATAAATGTCATACAGCTTTTCTTTTATGTTTTCTTCTAGTAGCTTTATAGTTTCATTTCTTATATT
TAAGTCTTTTACTTGTTTGGTTTTATAAACATATGGTATGAGAAAGGGCCCAATTTCATTCTTTTGCATA
AGGAATATTCAGTTTTCCCAACACCATTTATTGAAGAGATCGTCCTTTCCCCCATTGTTTATTCTTTGCA
CCTTTGCTGAAAATCAATTGACTATAAATAATTGGGTTTATTTCTGGGCTTTTTATCCTTTTCCATTAGC
CAATATATCTGTGTTTATGCCAATAGCATAGCTTTATATTTTGAAATGACAGAGTATTGCACCTCTAGCT
TTGTTCTTTTTGCTCAGATTGCTTTGGCCATTTAGCATCTTTTTTATTTTATTCAAATGTTAGGATTTTT
TACTATTTCTGTGAAAACTGACATTAGAATTTTGATAGGGATTGCATTGAATCTGTAGATTATTTTGGGT
AGTATGGACATTTTAACAATATTAATTCTTCCAACACATGAACATGAAATATATTACAATTTATTTGTGT
CTTCCTCAGTTTTTTTAAATCAACTTATAGTTTTCAGTGTACAGATCTTCAACTTTCCTGGTTAAATTTA
CTTCAAAGTATTTTATTTATTTATTTTGATGCTGTTTTAAATAGGATTAATTTCTTAATTTCTTTGTTGG
ATACCTCATTGTTAATGTATAGAAATGCTACTGATTTTTCTATGTTGATTTTGCAAACTGCAACTTTACT
GAATTTATGTATCAGTTTTAACAGTTCTCAGTAGAGTGTTCAGGGTTTTCTACATATAAAGATCATGTCA
TTAGCAAACAGAGATAATTTTATTTCTTGCTTTCTTACATGGATGTATTTTATTTCTTTTTCCTGCCTAA
TTGCTATGGATGGGATTTCTAGTACTATGTCAAAAAGAAGTGGTAATAGTCGATATCCTTGTCTTGTTCC
TGATCTTAGAAGAAAAGCTCCCAACTTTTCAGTTTTTGCATAATGTTAGCCGTGGGCTTGTCATATATGG
CTTTATTTTGTTAAGGTACATTCCTTCCATATCTGCTTTGTTGAGATTTTATTATGAACATATGCTGGAT
TTTATCAAATGCTTTGTCTGCATCTAGTAAGATGATCATATGATTTTTGTTCTTATTTTGTTAATGTAGC
ATATCACATTTATTGATTTATGTATGTTTAACCATTTTTGCATCCCTCAGATAAATCCCAAGTAATCATG
GTGAATGATGTTTTAAATGTGCTAGGGAATTCAGTTTGCTAATATATTGTTGTGAATTTTTGGATCTATG
TTCATCAAGAATATTGGCCTGCAGTTTTCTTTTCTGGTAGCATACTCTTAGGGTTTTCATATCAGTATAA
TACTGGCCTCATAAAATGAATTTGACCTCCTTTTCAATTTTTTGGAAGAATTTGAGAAGAATTCATATTA
CTTCTTTAAATAGAATTCACCAGTGACCTTTTCCTTTTTTTTTTTTTTGGTTAGGAGACTTAATTTATTA
CTGATCTATTTTCTTTATCCAGTATTGGTTTGTTCAGATGTAATATTTCTTGATTTAGTCTTGGTAGGTT
GCATGTTTCTAAAGATTAGTTCATTCCTTCTAGGTTATTCAATTTGATGGTGTATGGTTGTTCACATTAA
TAGTTTATGATTCCTTGTATTTATGTGTTATCAGTGTTAGTCTCCCTTCTTTCATTTCAGATATTGTTTG
AGTTGTCTTTTTTCCCTTGGTTAGTCTAGTTAAAGGTTTTTCGATTTTATCTTTTCAAAAAAACAACTTT
TTAGTATTTGCTTTGTTTATTTCTATCATTATCTTTGTTATTTTCTTCCTTCTGCAAAGTTTGGGGATAT
TCTGTTCTTCTTTTTCTAATCTCTAAGATGTAATATTAGGTTGTTTATTTGAGATTTTCCTTCTTTCTTT
AATGTAGGCATTTATTACTATAAATTTTTCTCTCAGGGCTTCTTTTTCTACATTCCATACTATTTGTTAT
CTTGTGTTTCCATTGTCATTGATCCCAAAATATTTTCTGTTTTTCCGTATAATTTCTTCTTTGACCCATT
GCCTGTTCAGGAGCATGTTGTTTAATTTCCACGTAATTATGAGTTGTTCAAGGTTATTCTTATTGACTTC
TAGTTTCATGTCATCATGAACAGAAAACATACTCGATATGATTTTAATCTTCTTAAATTTGTTAAAACTT
GTTTGGTGGCCTAACATGTGATCTATCCTGGAGAGTGTTTCATGTGATTTGAGAAGAATATGTATTTTGT
TGCTGTTGGATGAACTGTTCTATAAATATCTGTTAGATCTATTGGATTAAAGTGTAGTTCAAGCCTTCCT
CTAATAAGAAACTGCATAAATTATGTCAGGAGCAAGCCAGGGAAGAAGCCACCTTCCTGTTCTTGGTGGT
AGAGACCTTCCACATCTTTTAAAAGAAAGATTGTGGCCGGGCGCGGTGGCTCACGCCTGTAATCCCAGCA
CTTTGGGAGGCCGAGGCGGGCGGATCACGAGGTCAGGAGATCGAGACCATCCTGGCTAACACGGTGAAAC
CCCGTCTCTACTAAAAATACAAAAAATTAGCCGGGCGTGGTAGCGGGCGCCTGTAGTCCCAGCTACTCGG
GAGGCTGAGGCAGGAGAATGGCATGAACCCGGGAGGCGGAGCTTGCAGTGAGCCGAGATTGCGCCACTGC
ACTCCAGCCTGGGCGACAGAGCGAGACAGCGAGACTCCATCTCAAAAAAAAAAAAAAAAAAAAAGAAAGA
TTGTAGAGTTGGGATTGTCTCTGGAGGAAGCCAGGGAAGAAGGCGTGGGGAATGTTATTCCTCTTGTTTA
GGCTGAAGAAGGTTTATTCTTCATTTGCTATGTAGATTTCCAAGTACTGGAAGAGGCCAGAACCACAGGG
AGCGGCTGGAAAACGTGTAGCCTAATGGTTTCCATGGGGAAGTTGGAAGATGGTCTGATCCCAGGAATTA
AAGCTTCTGAGAGTGCTCAGGCAGTTCAAAACCCCCCATCGTTTTTTGTACTTGAGAGAAAACTCCAATG
GCTTATCTCTGCCATTCCTCAAGCATGGTGATCTAAAAGCCAAGCTCTGTGTCAGGGCCTATAAAAGTTG
GGTTCTACAAAATGGAAAACCAAGGCAATTATGAAGATGATTTTATAATATAGTTCATCTCTTCTCTCTT
TACACTCTAGAAAAAGCATTTTCTAGAAATAAATACATTTCCTTAAATTAAAAAAAAGTTGGGTTCTATA
TGTGTGCTATAGTTTCTCAACCCTTGGGAAGTAGCTGCAAATTTGGTTTTCTTCCCAAATGTAAGGCACT
GTGCTCAGGGTGGGGTTAGTGCATGAGTGTCTTTCCACTTTTTCTACCCATTTTGATATGGGTATTTTCT
GTCCTCTGGTGTGCATGAGTTTTTCTACTGGATTCTGGGTTTCTCTGGAGAGAACTGATCCATGTGTATG
TAGATGTTTACTTGGTACGTCCATGAGAGAAAAAATAGTCATAAACCTCCTATTCCACAATGTTGCTGAA
GTCACTTCCTTCAAAAGCTGTAATTTTCTTTTCTTACAGTGTCTTTGTCTGGCTTTGATATGAGAATAAT
TCTGGCCCCATAAAATGACTTTCGAAGTGTTCTTTCCTCTTCAAAGTTTTAAGAGAGTTTGAGAAGAATT
AATGTTGTTTCTTCTTTAAATGTTTGGTTGGACTTCTCCAGAGAAGCCGTCTGGTCCTGTACATTTCTTT
TTTGGGAGTTACTATCATTATCATTATTATTATTATTATTATTATTATTATTATTATTGGTTCAATTTGT
CTATTCAGTTATTCTATTTCCTCATGATTCAGTCTTGGTAGGTTGTATATTTTTGGAATTTACCTATTCC
TTCTAAGTTATCCAATTTGTTGGTGTATAATTGTTCATGGTAGTCTCTTTGATCCTTCTTATTCCCATAA
AATCAGTTGTAAATTCTCCTCTTTCATTTCTGGTTTTACATATTTGAGTCCTCTATCTTTTTTCTTAGTT
ATGCTTGCTAAGGATTTGTCTATTTTATCTTTCGAAAAAACCAATTCTTAGTTTTGTTGACTTTTTCTAT
TGCTTTTTTATTCTCTATTTCACTTACTTAGCTCTAATACTTATTATTTCCTTCCTTTTGCTGATGTTGG
GCTTAGTTTGTTCTTTTTCTAGTTCCTAGACATGTAAATCTATATTGTTTATATGAGATATTTCTTTTTA
TAATGTAGACATGTATCTCAATAAGATTTGCTTTTATTACTGCTTTTGCTGCATCTCATAAATTTTGGAA
ATTGTGCTTTCATTTTTGTTTGTTTCAAGATACTTTTAAATTTTCCTATTGATTTTTTTTCTTTGACCCA
TTGCTTTTTCAAGAGAGTGTTGTTTAATTTCCACATATTTGTGAATTTTTCAGTTTTCCTTCTATTACTG
ACTTCCAGTTTCCTTCCATTGTGGTCATAAAAGATATCTGGTATGATTTCAACTTCTTAACAGTTGTCAA
AACAAACTTCATGACCTGATATGTGATCTATCCTGAGAATATTCTGTGCGCACTTGAAAAGATGGTATAT
TAAGATGCTGTTGGGTAGCATATTCTGTGTGTTTGTGATCCATTTGGTCTATAGCGTTGTTCAAATCCAC
AGTTTGTTTATAGATTCCATATCTGGCTGTTCTGTTCATTATTGCAAGTCGGGTATTGATGTATCCTACT
GTTATGGTGTTGCTGCATGTTTATCCCTTCAGATCTGTCAATGATTGCTTTACGTATTTACATGCTTTGA
TGATGGATTCACATATATTTATAATTGTTATATCTTCTTGATGAATTAACTCTTTTATCTTATTTTATGA
CCTACTTTGTCTCTTGTGACAGTTTTTTATTTAAAATCTATTTTGGCTGATATAATTACAGGGTGTCCTC
TGTGTCTGTGGGTTTTACATCTGTAGATTCAGCCAATGCTGATTGAAAACACTGTATTCATGGAATGTGA
AATCCACAGATACAGAGGGTCAAATTTTTTTATCCACAGGTCCTGCAAGACTGACTGTGGGACTTAAGCA
TCCTCAATTTTGGTATCCCTTGGGGTTCTGAAACCATGACCCTGTAGATACTGAAGGACGACTGTATAGC
CCCATTGCTCTCTTTTGGTTACCATTTCTACAAAATGTATCTTTTTTATCAATTAACTTTCAAACTATGT
TTTCTTAAATCTGAAACGAGTATCTGGAGACAGCATATAACTGGATCTTGTTTTCATAAATCTATGCAGC
CATTGTAGGTCTTTTGATTGGGAAGTTTAATCCATTTGTATTTGAAATAATCATTGGTAGGTAATGACTT
ACTATTGCCTTTTTGTTAAATTTTTCTGTTTTGTAATTCTTTTTTTCTGTCTTTTTCTCTCTTAACTGTC
TTCTTTTGTGATTGGATGATATTTTATTGTGATATGCTATGATTTCTCTTTTTTATTTCTGTATTTTTAC
GTGGATTTTCCTTGTGGTTATCATGAGGCAATCACAAATATCTTATAATAGTCTACTTTAAGCTGATAAC
AACTTAACTTCAATCATATATCAAAAATCTTCACTTTTAATACCTCCCTCACACTTTTTGTTATGTCACA
ATTTACATCTTTTTATAATGTGTTTCTACAAACACATTTTTGTAATTTTAGTTATTCTTAATTATTTTGA
CATTTAACTTTTATATTAGAATTATAAATGATTTATATAATACTATTAGAGTATTACAGTATTCTCTATT
TGTCCATGTATTTACCTTTACTAGTGAGATTTGTGCTTTCATATGCTCTTGTGTTGCTCTTTACCATGCT
TTCGTTTCAACTTGAAGAACTCACTTTAGCATTTCCTATAAGATAAATCTAGTGGTGATGAACTCCCTCA
GCATTTGCTTGTATGAGAAAGTTTTTATCCTTCCTTAATTTTAAAGGACAGTTTTTCCAGGTATATTATT
CTTGGTCAGCAGTTTTTTTCTTTAAGTATTTTGAATATATACTTTCACTTTATTTGCTCTTTTAAAGTTT
CCACTGAGAAATCTGCTGTTCATCTTAAGAGAGCTCTTTTGGACATGACAATTCAGTTTTTTTCTTGCTG
CTTTCAAACTTCCCTTTCACTTTTGGCAATTTGATGATATTATGTCTCTAGTGGAGACTTTCTTTCTTTT
TTTTCCGAGATAGAGTTTCACCGTGTTGCCCAGGCTGGAGTGCAGTAGCATGATCTTGGCTCACTGCAAC
TTCCACCTCGTGGGTTCAAGCAATTCTCTCTGCCTCAGCCTCCCGAGTAGCTGGGATTATAGGCACCCAC
CACCACACCCAGCTGATTTTTGTAATTTTTGGTAGAGATGTGGTTTTGCCATGTTGGCCAGGCTGGTCTT
GAACTCCTGACCTCAGGTGATCCACTCCCGCTCAGCCCCTCAAAGTGCTGGGATTACAGGTGTGAGCCAC
CATGCCCAGCCGAGACTTCTTTAGATTTGTCCTATTTGGGATTCTTTGAGTTTCCTGAATTTAGATGTTC
ATATCTCTCCCCAAATTTGAACATATTTTAGTCATTTAAAAAATATTTTTACACCCCTTTCTGTCTTCTC
TGTGATTTCATAATGCATATATTGTTCCACTTAATGATGTCCCATGAACCTTCACTCATTTTTTTTTTTA
TTTCTTCTTGCTCCACTGACTGGATAATTTTAAATGATCTACCTTTTTTTTTTTTTTTGGTGTCCATCTT
TTGAGGGTTTTTTAATTTTATTTTTTATTTTTTTATTTTTATTTTTTATTATACTTTAAGTTTTAGGGTA
CATGTGCACAACGTGCAGGTTAGTTACATATGTATACATGTGCCATGTTGGTGTGCTACACCCATTAACT
CATCATTTAACATTAGGTATATCTCCTAATGTTAGGTATATCCCCCTCCTCCCCCCACCCCACAACAGGC
CCTGGTGTGTGATGGTCCCCTTCCTGTGTCCATGTGTTCTCATTGTTCAATTCCCACCTATGAGTGAGAA
AGTGCAGTGTTTGGTTTTTTTCCTTGCGATAGTTTGCTGAGAATGATGGTTTCCAGCTTCATGCATGTCC
CTGCAAAGGACTTGAGCTCATCCTTTTTTATGGCTGCATAGTATTCCATGGTGTATATGTGCCATATTTT
CTTAATCCAGTCTATCATTGTTGGACATTTGGCTTGGTTCCAAGTCTTTGCTATTGTGAATAGTGCCGCA
ATAAACATACGTGTGCATGCGTCTTTATAGCAGCATGATTTATAATCCTTTGGGTATATACCCAGTCATG
GGATGGCTGGGTCAAATGGTATTTCTAGTTCTAGATCCTTGAGGAATCGCCACACGGACTTCCACAGTGG
TTGAACTAGTTTACAGTCCCACCAACAGTGTAAAAGTGTTCCTATTTCTCCACATCCTCTCCAGCACCTG
TTGTTTCCTGACTTTTTAATGATCACCATTCTAACTGGTATGAGATGGTATCTCATTGCGGTTTTGATTT
GCATTTCTCTGATGGCCAGTGATGATGAGCATTTTTTCATGTGTTTTTTGGCTGCATAAATGTCTTCTTT
TGAGAAGTGTCTGTTCATATCCTTTACCCAGTTTTTGATGGGGTTGTTTTTTTCTTGGTTTGTTGATTCT
TTCTCCAGTTTGATCAAGTCTGCTATTGAATCCTCTAGGGAGTTTTTTAGTTCAATTATTGTATTCTTCA
ATTCCAGAGTTTTTACTGATATTTGACTTTTTGTGTGTGTGTGTATAGTTTCTTTCCCTTCATTGATATC
CTCATTGTGTTCATGTATCATCTTCCTGATTTTGTTTAGTTGTCTATCTCTGTTCTCTGGTACTTTGCTA
AACTTCATTAGGATAATTATTTTGAATTCTTTTTTAGGCAATTTATAAATCTCTATTTCTTTAGGGTCAG
TAACTGCGGATTTCTTTTATCCCTTTTTGTTGTTTCCCTAATTCTTTGTGTTCCTTGTGCTTTGCATTGG
TGTCTGTGCATTGGAAGAATAGCCACCTGTCAGCCATCCGACACAATGTCACAAAGGACACAAAACAGCC
ACCAAGATTTATGGACTGACTTTGACAGGGCAAGACCTTTGCCAGTCAGCCTGGCTAGAGATACTGAGAG
TCTCTCAAACAGCTAATCTACCGTCCCCTTTTCCTATGTTTGTAGCTGTATCAATTGTTCTAATCATACT
GGTTCTTTCAGTGCTCCATATGAGGCAAGAGTGAAACTGGACCCTAGTGCAGCACACTGAATGGCAAGGA
CATTTGATGTGCTCCACTCCTCTCCCTCCCTTGAAAGTGGTATCTTGATGAAAATGAATAAATTAGCCCC
TAGAAAGGATGGATAAATTGTTTCAAACCAAGTAACAGGATACCAGCACAGTTATGATTGAAATGCCTGT
TTGATTTGAAAACCCATATCTTTTCCACAATGCAATGTTGCCACTTACTGCTTTTTAACTTCAATAATTT
GATCAAAGCTTATGGCTCTATCAACTACCCATGCCTCTTTCTAATAACCAACCCTATTTTACTAGTTTTT
CCAACTATTCATCATCCTCATAAGTACTTTTTTCACCTCTAATGATTTCATGTGAAATTAGTTTTTAATA
ATCATAAAACATACTACTATTAGAACAAAAGTCATTAATGAGTTCAAGTTATAAAAAGGAAGATTACATG
ATATACAAATATATAATTACAAATTTGAGCAACTATGGAGTAAATGTTAACAATAACCTCATTTCATTAA
CTCTCATCTTATCAGGAAGTTTAATAGCACCAACAGTAATAGTAACTAAAATTTAATGAAGACTTACCAT
GTGCCAGATATTATGCTAAGTATTCTTCATGCATTACCTCAATTTATCTTCTCAGCAAACACAAATGCAG
TGACTACTCTTATAATGATGAGGTGGTTTTACTTAAAGAATTAAGAAACTTACCCAAGGACAAAAGAAAG
GACTTGAACAAAGATGATCTGACACCAGAAGTGAAGAAGTCTTTATTACTATGTAATACAGCCTCAATGT
AGATTGTAACACAGAAAAAAACTAGCCTGACTTACAGAATTCCTTCCACCTAGGTTTTAAAGGGATACAT
TTTGCACAAGGCCTAAATAAGCTATAGGAAAACAAAACACCCACGCAAGTTTCTCTATTGAATTAATTCA
TAATCTCCTATTAGTTTTATCCCATTCTTCATCATATTGACAACCAGGATATCATTAGGGGAAAACAAAA
TCTATTACTTTGATTTTCTCTTCAAAGTTGGAGTTTTCTCCTCAGTTTTTCTCCAGCTTCTTCTGTCAAG
TTCTTGGCATGCTGTAATTTTCAGTCCCCATTAATGAACAGTAAAGGATCTGGGTTTCTCTTGACATTGA
CATGAAGTCTGTCTCTCTCATGCCTTGCCAGTAGGTGAGATGAATATGAAAATTACATAAAGACTGCATA
TTAAATAGAGGAAAGCTGAATTAAAACACTCTAAGGTGGCCGGATATGGTGGCTCACGCCTGTAATCCCA
GCACTTTGGGAGGCCAAGGCTGGCAGATCACTTGAGGCCAGGAGTTCAAAACCAGACCAGCCAACATGGT
GAAACCCTGTCTCTACTAAAAATACAAAAATTAGCCTGATGTGGTGGCATGCACCTGTAATCCCAGCTAC
TTGGGAGGCTGAGACATGAGAATTACTTGAACCCAGGAGGTGGAGGTTGCAGTGAGCCAAGATGGTGGCA
CTGCACTCCAGCCTGTGCCTCAGAGCAAGACTCCATCAAAAACAACAACAACAACAACAACAACAACAAA
AACACTCTAAGGCAGAAAGGGAAGTCTGGTTACCATTAGGTGTTTGATGAATAAGAAAATTCAATATAAT
GGGAAGCTTCCCATTCAATAGCAAACAATTAAAAAAATAAGTAAATAAGAGGAAGCTGAATATTACCAAA
TCCAAGAGAATAAAGGGAAACCATAACCAGGTCATCTTTAATTCCAGCACCCTACAAGTTAAGTAATGAT
CTCAGAACTATAAATCCTTGAAAATAAATTCTTAATTATATGGATTATACTAATTTATGCTGTGACTTAA
AAAGGTGAAATTATAGACTAATGTAGAATATACATAAATCTGTAAAACTTACAGCTAACTTAAGAAATTA
GCAGAGAACAAAGGGAGCAACTGAAGCTTCTAGCCCCCCTTTGTAAGATTTAGGCTTTTCCAGTCAAAAA
CCTCATTTAGATGTTATGTTATTTACTGTTGAATTTTAAAAGTTATATTTTCTTTTATGTAATCAATTGG
AAAGTCCTTCAGTTTGGGTATGATAATTGATAACTTCCTCAGTACCTAGAAATCTATCAAGGATAATGTT
GACATCCAATAAAGTGTTTATGTTGAAATCCTTTATTTAGAATTAAAAAGGTAGTCAAAGAGTTTCTTGA
AATAGAGATATAGAAATGTATATTATCATTTTAGTTTATATTCATTTTTTATGTAAAGGAATAATGGTAT
ATTAAAATGAATATAAAATTAATACAATTTTGGTTATTGTATGTAGCCCCTATTCTTTTTTTATTATTAT
ACTTTAAGTTCTAGGGTACATGTGCAAAACATGCAGGTTTGTTACATAGGTATACATGTACCATGTTGGT
TTGCTGCACCAATCAACTCATCATTTACATTAGGTATTTCTCCTAAGTAATCCCTCCCAAAGCTCCCAAC
CCCCTAACTGCCCCTGGTGTGTGATGTTCCCCTCCCTGTGTCCATGTGTTCTCATTGTTCAACTCCCACT
TATGAGTGAGAACATGCAGAGTTTGGTTTTCCGTCCTTGTGATATTTTGCTGAGAATGATGGTTTCCAGC
TTCATGCATGTCCCTACAAAGGACATGAACTCATCCTTTTTTATGGCTGCATAGTATTCCATGGTGTATA
TATGCCACATTTTCTTTATCCAGTCTATTATTGATGGACATTTGGGTTGGTTCCAAGTCTTTGCTATTGT
GAATAGTGCTGCAATAAATATACATATGCATGTGTCTTTATAGTAGCATGATTTACAATCCCTTGAGTAT
ATACCCAGTAATGAGATTGCTGGGTCAAATGGTATTTCTAGTTTTAGATTCTTGAGGAATCATCACACTG
TCTTCCACAATGGTTGAACTATTTTACACTCTCACCAACAGTGTAAAAGCGTTGCTATTTCTCCACATCC
TCTCCAACATCTGTTGTTTCCTGACTTTTTAGTGATTGCCATTCTAATTGGCATGAGGTGGTATCTCATT
GTGGTTTTGATTTGTGTTTCTCTGATGACCAGTGATGATGAGCATTTTTTCATATGTCTGTTGGCTGCAT
AAATGTCTTCTTTTGAGAAGTACCTGTTCATATCCTTTGCCCACTTTTTGATGGAGTTGTTTGTTTTTTT
CTTGTAAATTTGTTTAAGTTCTTTGTAGATTCTGGATATTAGCCCTTTTGTCAGATGGATAAATTGCAAA
AATTTTCTCCCATTCTGTAGGTTGCCTGTTCACTCTGATGATAGTTTCTTTTGCTTTGCAGAAGCTCTTT
AGTTTAATTAGATCCCATTTGTCTGTTTTGGCTTTTGTTGCCATTGCTTTTGGTGTTTTAGTCATGAAGT
CTTTGCCCATGCCTATGTCCTGAATGATATTGCCTAGAGTTTCTTCTAGGGTTTTTATGGTTTTAGGTCT
TACATTTAAGTCTTTAATCCATCTTGAGTTAATTTTTGTACAAGGTGTAAGGAAGGGATCCAGTTTCAAC
TTTCTACATATGGCTAGCCAGTTTTCCCAGCATCATTTATTAAATAGGGAAGCCTTTCCCCGTTGCTTGT
TTTTGTGAGGTTTGTCAAGGATCAGATGGTTGTAGATGTGTAGTGTTATTTCTGAGGCCTCTGTTCTGTT
CTGTTGGTGTATGTATCTGTTTTGGTACCAGTACCATGCTGTTTTGGTTACTGTAGCCTTGTAGTATAGT
TTGAAGTCAGGTAGTGTGACGCCTCCAGCTTTGTTCTTTTTGCTTAGGATTATCTTGGCTATGCAGGCTC
TTTTTTGGTTCCATATGAACTTTAAAGTAGTTTTTTTCCAATTTTGTGAAGAAAGTCAGTGGTAGCTTGA
TGGGGATAGCATTGAATTTATAAATTACCTTGGGCAGTAATGCCATTTTCATGATATTGAGTCTTCCTAT
CCATGAGCATGGAATATTCTTCCATTTGTTTGTGTCCTCTTTTATTTCGCTGAGCAGTGGTTTGTAGTTC
TCCTTGAAGAGGTCCTTCACATCCCTTGTAAGTTGGATTCCTAGGTATTTTATTCTCTTTGTAGTAATTG
TGAATGGGAGTTCACTCATGATTTGGCTCCCTATTCTTTATAAAAGTTCTAAATATTTGCCAAATCATCA
AAGAAGATATTCATTACTTTTAATTCTTACAAATTTTCTTAGCAATAAAAGTCACAGTAAGACAGCTAGC
AGATAAAAAAGATAAAAGTACATAGTTTAAAACAACACTGATTAGCACACCAAAAGAATATTGAGCCAAT
CCTTATTATATGTAATACAAATATTTATATATGCACATTTAACTGTGTTTGGAGACCCATGTTTCTACAC
ATATGTAATTATTTAAGATATATTTTATGTATAGCTATCATGAACATAATAAAATACATTTTATTTCTAT
ATTAACACATTTTAACCTATTTTAGTAATCTATAGAAAGATGTAGACAATGATTCTGGTTTTAACTACAT
TTATTTTTTGTTTGTTTCTTTACCTTGGAGGTCAAGACATCTTTATGACAGAAGAACAGAAGAAATACTA
TAATGCAATGAAAAAGCTGGGGTCCAAGAAGCCACAAAAGCCAATTCCTCGACCAGGGGTAAAAAAATAT
ATATATCTTTAGCATATAGATTTTCAAATTATTTCTAATTCATTTTTAATGCACATCTTTAATTTCTGGA
TAATACTTGAAAAGTTTACTCTGCATTCGATATTATTCTTATTTCTTTGCAGAACAAAATCCAAGGATGT
ATATTTGACCTAGTGACAAATCAAGCCTTTGATATTAGTATCATGGTTCTTATCTGTCTCAACATGGTAA
CCATGATGGTAGAAAAGGAGGGTCAAAGTCAACATATGACTGAAGTTTTATATTGGATAAATGTGGTTTT
TATAATCCTTTTCACTGGAGAATGTGTGCTAAAACTGATCTCCCTCAGACACTACTACTTCACTGTAGGA
TGGAATATTTTTGATTTTGTGGTTGTGATTATCTCCATTGTAGGTAAGAATATTTATTTTTCAGATTTTA
TTTTTTGAGTAAAGCTAAACTTCACTTATGCTCAAGGAAGTATATGCTGAATTCTTGAACACAAATTTAC
TCTTGAGTAAGTCAGTACAATGAAAGAATGAAAAAATCCTAAAAAGCAAAACAAATATCTGTCTCTCATA
CTACAGTGTTTCTGGGTTTACACTACAGGCCCCAAAGCCAAATATTTCTATCTATTAGAAGGAAATTATA
CTTGTTGATATTTAGTTTCCAATATTACCTTTGCAAAACTAGTTACCATGTTTATATATAGCTGTGTCTT
GGAAAACCAATGTTGAATCTGAAATAACTTCTGGTTTTAGCACATGTGAGACTTGTCATTTGATTAATGA
TAATATTAATAGCCAGAATATCTGTGAATTTGTGAAAATTCTTCCAGAATTTAAAGTGAAGTTAAGATCT
CTAGAAATGCCAGTTTTATTTTTTACAGCTTAATACTTCACAAGTGTATAAAATGCCAATCATTCCTCAA
CCAGCCATGGTGTTCATGTCTGTGAATAACAGCACATGATTTTATACTTCCTATACATTCATTGCCATGG
TTGTTTTTCTATCTTCTTGTTTTTGTTGTTGTTGTTATTTTCACATATCTTGCAAAGTATTTTAGGATTG
TATTTCTTTTTAGAATTATAAACTTTTAAGAGAGAGAGATTATAGAGATTACTACAAGTTTAATCAACAG
GTTATCAATCTGAAAAGGTTCAAGGTTGCATGACTTAGAATCATAAAATTGAGTGCCACATAACAGAGTA
AGCATTTAAAATCAATAATTTTGGAAAAGCATATTATCACCAGAAGTCATACCAAAATTGATATAAGAAA
ATGTATTAAAATTTAGGTGAGAATTTCACACTAACAAAAATGATTCTGAAAGCATTCCTATTAAGTCAGG
AAAAAAAAAAGAGTGTGCCTGTATCATCCCTGTTTTTAGTGTTGTTGGCCAATGCAATTAGATAAGAAAA
CAGAGAGGTACATATATAAAAACAATGGAGTAAATGATCCTTAATATAATGACCTTCTTCATATAATGAA
AGAACTTGTTCATATATTGAAAGAGAATCAACTAAAAATAAATTCAATTAGAGTTCAAAAGGTACAAATT
TTAAATTACATGTGCCAACACTAATAATTATCTTATACATGAAAAAAATTAGAAAATCTGATTCACAATT
GCAATAAAAATAAAGTAAATTATCTAATAATAACTTTATCATATGAGACTTCTGAAGAAAACTTTAGGAT
ATAAAAGAAAGCTAAAATAATTGAAAGGATTGTCATGTACTTAGATAAGCTTCTCTCATTGTAATTAAGA
CTTTCAAATTTATTAATCTATAAATTCAAGGAAAATATCAATGAGTCTTTTTATGTAGTGTGGGTAATAA
ATAGACAAAAACAATATAAGTCTAAATGATTCTAAATTCATCTGGAAGAAAATTAGGGTGACAAGAGGTG
GCTTATTCTGCAAAATATTAAGACATATAGATGTTAAATAAAACTGTTCAAATATAAAAGCTCAGTGGGT
TTGAAGAGAGTCCAAATTAAACTTTAATGTATAATTACCAATTTACTGTGTGAAAAGATCACATGAATCA
AAATCTAAAGTTAAAAAAAAAGATTCTAGTAGGAAATATGTGGAAATAGACATATAACCATATACTCTTA
TAGTCCTTTCTATGCAAAACACAAATTTGTAAAGCCAAAAAAAAAAATGAAGAAGAAATGACTGCACAAA
AAAACAAAATATTCTTTGGGGAAAAAATCATCATAAATAAAAGGTTGAAAGACAAAAAATAGGTGAAATG
TTTACATAACAGTTAAGGCCGAGAAAGGATCAATACTTTAAATAGAAAATTGTTCTTATAAATTAATAAG
AATATAGTAAAAATCCCAATAGACACATGGGCAAATACCAAACTAAGCCAAAAACAGAAGTTAAAAACAA
CCAATAAGGATACAAAATAACAAAACACAAGGAAAATTGTGGATTATTTTCACAAAAAATGCAAATAAAT
TACAATAGTAGATGTTTTCAAATGTCATGAAAATTTTTTGGTAAAAAACTATAAAGAAGAATGGTAGCAC
TCAATACTGTTGAATGTGTAAAGTATGAGCACTATCATATACTGCCATTAGAAATGTTAATTAGTACAGG
CTTTCTGGAGATAGGTTCAGCTATATATTGTGTGTATATATATACACATACACACACATACTATGTATAT
AGTATAGAGTATGTATATATGTATACTATATATATACATACTCTATACTATATATATAGTATACGCATAC
TCTATACTATATATATAGTATACGCATACTCTATACTATATATATAGTATACGCATACTCTATACTATAT
ATATAGTATACGCATACTCTATACTATATATATAGTATACGCATACTCTATACTATATATATAGTATACG
CATACTCTATACTATATATATAGTATACGCATACTCTATACTATATATAGTATATGCATACTATATGCTA
TATAGCGTATATGCATACTATATGCTATATAGAGTATACGCATACTATATGCTATATATACATACTATAT
AGTATACATATACTATGTACTGTATATACGTATACTTAGTATATGTATATGTATATGTATAGTATATGTA
TATGTATAGTATATGTATACTATATACTGTATATAGTATATATAGTATGTGTATATATATAGTATGTGTG
TATATATAGTGTGTGTATATATATAAACATATATATACTGTTGACCCAAGTTCTATTAACATATTATTAA
AAAGTACTCATGCAATTAGGAGAACATAGCTATTTAGATATTCATTTCAGTAACAAAAATGGAAACAGTA
TAAATGCTAGTCTTCAATTTGTGGTTATTTTGAGAGAACATTATACCACTATAATAAATTATTTCGTTGT
CATTTTTATGAAACTATCTCCATGACATATAAAGTAAATAAAATAGGTTACAGGCTGGGCACTGTGGCTC
ACGCCTATAATCCCAGCACATTGGGAGGCCGAGGTGGGCGAAATCACTTGAAGTCAGAAGTTTGAGACCA
GCCTGGCCAACAAGGTGAAAACCGTCCCTACCAAAAACACAAAAATTAACCAGGTGTGGTGGCGCATGCC
TGTAGTCCCAGCTACTCTGGAGGCTGAGGCTGGAGAATCATTTGATCCCGGGATGCAGAGATTGCAGTAG
GCCAAGATCACACCACCGCACTCCAGCTTTGGCGACAGAGCAAGACTCTGTCTTGAAAAATAAATAAATA
GGTTACAAAAAAAGCTTATATAACATGAATACATTTGTTTGTGTGTGTACATGTGCTTGCACAGAAAAAT
GTCTGAAGTGATACTCAAAAAAATATTGATAATAGTTATTGTTGACATTTCAGGTGATTTATATTTTCTT
TTTAGCATTTAACGGTATTGCTTGAATTTTCTATGTGAGTAGGTATCATTCTTAAAATGTAAAACACTAT
TATGGAGGGAAAAAATGGCCCATGAACTTAGTAAAAAGGAGAAAATGTCACAATGGTAATGTTAGTCATT
TCTCTTTGGAATTTCTTAATCCTTTTTTAAGTAATTGCATTCAGTATAAAATAACTGTCTTGGCCGGGCA
CGGTGGCTCACGCCTGTAATCCCAGCACTTTGGGAGGCCGAGGCGGGCGGATCACGAGGTCAGGAGATCG
AGACCATCCTGGCTAACAAGGTGAAACCCCGTCTCTACTAAAAATACAAAAAATTAGCCGGGCGTGGTAG
CGGGCGCCTGTAGTCCCAGCTACTCGGGAGGCTGAGGCAGGAGAATGGCGTGAACCCGGGAGGCGGAGCT
TGCAGTGAGCCGAGATCGCGCCACTGCACTCCAGCCTGGGCGACAGAGCGAGACTCCGTCTCAAAAAAAA
AAAAAAAAAAAAAAAAAAAAAAAAAAAAAAAACTGTCTTGAATTCATAAGAAATGAGTTGACAGATAAAA
CTGTTTAGAGTCATCATTTCAGGTAGCATACATCTTTAAATATTTTATTTCTATTATTTTCCTCCACATA
CAGGTATGTTTCTAGCTGATTTGATTGAAACGTATTTTGTGTCCCCTACCCTGTTCCGAGTGATCCGTCT
TGCCAGGATTGGCCGAATCCTACGTCTAGTCAAAGGAGCAAAGGGGATCCGCACGCTGCTCTTTGCTTTG
ATGATGTCCCTTCCTGCGTTGTTTAACATCGGCCTCCTGCTCTTCCTGGTCATGTTCATCTACGCCATCT
TTGGAATGTCCAACTTTGCCTATGTTAAAAAGGAAGATGGAATTAATGACATGTTCAATTTTGAGACCTT
TGGCAACAGTATGATTTGCCTGTTCCAAATTACAACCTCTGCTGGCTGGGATGGATTGCTAGCACCTATT
CTTAACAGTAAGCCACCCGACTGTGACCCAAAAAAAGTTCATCCTGGAAGTTCAGTTGAAGGAGACTGTG
GTAACCCATCTGTTGGAATATTCTACTTTGTTAGTTATATCATCATATCCTTCCTGGTTGTGGTGAACAT
GTACATTGCAGTCATACTGGAGAATTTTAGTGTTGCCACTGAAGAAAGTACTGAACCTCTGAGTGAGGAT
GACTTTGAGATGTTCTATGAGGTTTGGGAGAAGTTTGATCCCGATGCGACCCAGTTTATAGAGTTCTCTA
AACTCTCTGATTTTGCAGCTGCCCTGGATCCTCCTCTTCTCATAGCAAAACCCAACAAAGTCCAGCTCAT
TGCCATGGATCTGCCCATGGTTAGTGGTGACCGGATCCATTGTCTTGACATCTTATTTGCTTTTACAAAG
CGTGTTTTGGGTGAGAGTGGGGAGATGGATTCTCTTCGTTCACAGATGGAAGAAAGGTTCATGTCTGCAA
ATCCTTCCAAAGTGTCCTATGAACCCATCACAACCACACTAAAACGGAAACAAGAGGATGTGTCTGCTAC
TGTCATTCAGCGTGCTTATAGACGTTACCGCTTAAGGCAAAATGTCAAAAATATATCAAGTATATACATA
AAAGATGGAGACAGAGATGATGATTTACTCAATAAAAAAGATATGGCTTTTGATAATGTTAATGAGAACT
CAAGTCCAGAAAAAACAGATGCCACTTCATCCACCACCTCTCCACCTTCATATGATAGTGTAACAAAGCC
AGACAAAGAGAAATATGAACAAGACAGAACAGAAAAGGAAGACAAAGGGAAAGACAGCAAGGAAAGCAAA
AAATAGAGCTTCATTTTTGATATATTGTTTACAGCCTGTGAAAGTGATTTATTTGTGTTAATAAAACTCT
TTTGAGGAAGTCTATGCCAAAATCCTTTTTATCAAAATATTCTCGAAGGCAGTGCAGTCACTAACTCTGA
TTTCCTAAGAAAGGTGGGCAGCATTAGCAGATGGTTATTTTTGCACTGATGATTCTTTAAGAATCGTAAG
AGAACTCTGTAGGAATTATTGATTATAGCATACAAAAGTGATTCAGTTTTTTGGTTTTTAATAAATCAGA
AGACCATGTAGAAAACTTTTACATCTGCCTTGTCATCTTTTCACAGGATTGTAATTAGTCTTGTTTCCCA
TGTAAATAAACAACACACGCATACAGAAAAATCTATTATTTATCTATTATTTGGAAATCAACAAAAGTAT
TTGCCTTGGCTTTGCAATGAAATGCTTGATAGAAGTAATGGACATTAGTTATGAATGTTTAGTTAAAATG
CATTATTAGGGAGCTTGACTTTTTATCAATGTACAGAGGTTATTCTATATTTTGAGGTGCTTAAATTTAT
TCTACATTGCATCAGAACCAATTTATATGTGCCTATAAAATGCCATGGGATTAAAAATATATGTAGGCTA
TTCATTTCTACAAATGTTTTTCATTCATCTTGACTCACATGCCAACAAGGATAAGACTTACCTTTAGAGT
ATTGTGTTTCATAGCCTTTCTTCTTTCATATCCCTTTTTGTTCATAGAATAACCACAGAACTTGAAAAAT
TATTCTAAGTACATATTACACTCCTCAAAAAAAACAAAGATAACTGAGAAAAAAGTTATTGACAGAAGTT
CTATTTGCTATTATTTACATAGCCTAACATTTGACTGTGCTGCCCAAAATACTGATAATAGTCTCTTAAA
CTCTTTTGTCAAATTTTCCTGCTTTCTTATGCAGTATTGTTTAGTCATCCTTTCGCTGTAAGCAAAGTTG
ATGAAATCCTTCCTGATATGCAGTTAGTTGTTTGACCACGGTACATACTTGAGCAGATAATAACTTGGGC
ACAGTATTTATTGCATCACTTGTATACAATCCCGTGTTTGGCAAGCTTTCAAATCATGTAATATGACAGA
CTTTACACAGATATGTGTTTAGTATGAATAAAAAAGCATTGAAATAGGGATTCTTGCCAACTTGCTCTCT
TGCCACCAACTTACTTTCCTAAATTATGGAAGTAATCTTTTTTGGATATACTTCAATGTATACAATGAGG
AAGATGTCACCTTCTCCTTAAAATTCTATGATGTGAAATATATTTTGCCTCAATCAACACAGTACCATGG
GCTTCTAATTTATCAAGCACATATTCATTTTGCATTAGCTGTAGACATCTAGTTTTTTGAAAACACCTAT
TAATAGTAATTTGAAAAGAAATAACCATAATGCTTTTTTTCGTGAGTTTATTTCAGGAATATGAGATCTT
TCTTCTATAAAGTTATTCATGCACAGGCAAAAATTGAGCTACACAGGTAGAATGTAGTTTTACTTAGAAG
ATTTTTGTGGGAGGTTTTGAAGCAAATATATAAAACAACTTTCACTAATTTGCTTTCCATATTTAAAAAA
TAATAAATTACATTTATATAATAAATGTTTAAAGCACATATTTTTTGTTGTTCTGGCAATTTAAAAAGAA
AGAGGATTTAAACGTACCTATAGAAACAAAGATTTATGGTTAAAGAATGAGATCAGAAGTCTAGAATGTT
TTTAAATTGTGATATATTTTACAACATCCGTTATTACTTTGAGACATTTGTCCTAATCTACGTATAAAAC
TCAATCTAGGGCTAAAGATTCTTTATACCATCTTAGGTTCATTCATCTTAGGCTATTTGAACCACTTTTT
AATTTAATATGAAAGACACCATGCAGTGTTTTCCGAGACTACATAGATCATTTTATCACATACCTACCAA
GCCTGTTGGAAATAGGTTTTGATAATTTAAGTAGGGACCTATACAAAATATATTACATTTATCAGATTTT
TAAATACATTCAATTAAGAATTTAACATCACCTTAAATTTGAATTCAATCTACCGTTATTTCAAACTCAC
AAATATAACTGCATTATGAATACTTACATAATGTAGTAAGACAAGATGTTTGACAGGTTCGTGTGTAATT
TTCTATTAATGTTTTTACATTGCCTTGTTTTTATGTAAAATAAAAAATATGGGCAACTGGTTTGTTAACA
ACACAATTTCTTCTTAGCATTTCAAAAATATATATAAAGTTGTTCTTTTTCCTATTTCATGAACTATGTT
TTTTTTTAAAATAACATGGTTAAGTTTTATATATATTTACGTTTGTTTCAGGAATGTCTACTTGTGACTT
TTTATCAATTAAAAATAATATTTGGAAGAAAGAGCTTATTAAGTATAAGCTTGAAGTAAAATTAGACCTC
TCTTTCCATGTAGATTACTGTTTGTACTGATGGTTTCACCCTTCAGAAGGCACTGTCATATTAATATTTA
AATTTTATAATCGCTGAACTTATTACACCCAACAATACAGAAAGGCAGTTACACTGAAGAACTTAACTTA
GAATAAAATGGAAGCAAACAGGTTTTCTAAAAACTTTTTTAAGTGACCAGGTCTCGCTCTGTCACCCAGG
CTAGAGTGCAATGGCATGATCATAGCTCTCTGCAGCCTCAACTCTGGGCTCAAGCAACCCTCCTGCCTCA
GCCTCCCAAGTAGCTAAGACTACAGGTACATGCCACCATGCCTGGCTAATATTTAAATTTTTGTAGATAA
GGGGTCTTGCTATGTTGCCCAGGCTAGTCTCAAACTCCTGGCTTCAAGTGTTCCTACTGTCATGACCTGC
CAACATGCTGGGGTTACAGGCATGAGCCACCATGCCCCAAACAGGTTTGAACACAAATCTTTCGGATGAA
AATTAGAGAACCTAATTTTAGCTTTTTGATAGTTACCTAGTTTGCAAAAGATTTGGGTGACTTGTGAGCT
GTTTTTAAATGCTGATTGTTGAACATCACAACCCAAAATACTTAGCATGATTTTATAGAGTTTTGATAGC
TTTATTAAAAAGAGTGAAAATAAAATGCATATGTAAATAAAGCAGTTCTAAATAGCTATTTCAGAGAAAT
GTTAATAGAAGTGCTGAAAGAAGGGCCAACTAAATTAGGATGGCCAGGGAATTGGCCTGGGTTTAGGACC
TATGTATGAAGGCCACCAATTTTTTAAAAATATCTGTGGTTTATTATGTTATTATCTTCTTGAGGAAAAC
AATCAAGAATTGCTTCATGAAAATAAATAAATAGCCATGAATATCATAAAGCTGTTTACATAGGATTCTT
TACAAATTTCATAGATCTATGAATGCTCAAAATGTTTGAGTTTGCCATAAATTATATTGTAGTTATATTG
TAGTTATACTTGAGACTGACACATTGTAATATAATCTAAGAATAAAAGTTATACAAAATAAAA
>NG_031891.2: 5001-101665 Homo sapiens sodium voltage-gated channel alpha
subunit 10 (SCN10A), RefSeqGene on chromosome 3
SEQ ID NO: 312:
ATGGAATTCCCCATTGGATCCCTCGAAACTAACAACTTCCGTCGCTTTACTCCGGAGTCACTGGTGGAGA
TAGAGAAGCAAATTGCTGCCAAGCAGGGAACAAAGAAAGCCAGAGAGAAGCATAGGGAGCAGAAGGACCA
AGAAGAGAAGCCTCGGCCCCAGCTGGACTTGAAAGCCTGCAACCAGCTGCCCAAGTTCTATGGTGAGCTC
CCAGCAGAACTGATCGGGGAGCCCCTGGAGGATCTAGATCCGTTCTACAGCACACACCGGGTAAGAGCTA
CTAGTAGGAATGTCTGCTCTCAGCTCTTGGAGAGGAAGTCTCGGTCCCACCCAGCCCAGAAAGTCCTCCT
TGGCCCTGGGTGATGTGGCAGTCTGCTAAAGATTCCGTTAACAACCAAAAAAAATATATATATATATCTC
TAACTTATTTGATGATCTCTTTGAATTTTTATGACATTTATGATAGAAACTAACTAAACCCACATCTTCA
AAAGCAGATAACTGCTGCAGAGCTTTCAGGGAAAGTTGGCTGTTCCCTTGGATGACCTCTGGCCTGGGGC
CTCCAGAGGAGTTGGGTCTCTTTGCAGGTGGAGAGCAGACATCTTGAGGTTGGGGGAGGAGCATGGCGGT
GAGGACAGAAAGCAGGATGAATTTAAGCCAGTGGTCCTCAACTATGATCTTGGAACCCCTGGTGTCCCCA
AAACTGTTTTAGTGGGTTTATAAAATGAAAACTATTGTATAACAATACTTGGATGTAATTTGTCTTTTTC
ACACACTTTCTCTTATGATCATACATTGGTGAATATAGAGGCTATTGATGGGGGATGATGTCATCACTCT
AAAAGCTACTGGAATGTGTGCCTGTATATTCCCGTGTTTTAAAATTTCCTGAGTTTTGAATTTCTAATAT
GAGATATATATACACACACATATGATGTATATATGTGTGTATACATCTATACACTATATATATACATACA
ACTATGTATATACATACAACTAGACCATGTAGCTAGTTATGTATATATAGGTATATAAGCATATATACAT
ATATATTAATTTCTAATATGATATACATATATCTGGCATAAACAAAAAGGTATCAACATAAACAAAAGTT
ATTTGGGGTCCTCAATAAGGTTTGAGAGTAATAATCCCTGAAATCAAAAAGTTTGAGAATCAGTAGTTTA
AACAAGAGCTTCTTCATGACAATTATCTCACCTGTGTTGTATTTTTGATGTTCTACGAGCCAAAAAATGG
TGAATGCATGTTTGGGAGTTAGGAGGACAGGAATCAGCCATCTCAAATATGGCTAGCAGAGTAAATAAAA
AGGCACTGAATATCTTTCTTCCTCCTCTCCCATGCCAAGAGTTCATATCCTCCTAAATCCTCAGAGGGAG
AAACCAGGAGAGGATTTAAGAGACAGAGATGTAGAAGGTGGAGACAGAAGAAACATTACAAAACAAGGAT
GGTTCTCTTGTCAGAAGAGAAATTAAATGCAACCATAACTTCACCATCCTGTTGCCAGAGTCCTCCCCTG
GGCATAACACCTCTTACCAGCCTGAGGATTGGCTGTCTGTCCATTAAAGTACCCTCAGTGGGCTCATCTT
GGTGCACTGTCAGTCCCAGAGATGAGGATCTTGCTGAGAGGGTGAGGATATGGCTTTTAGTCCTTGATCC
TCTCTGCCTCTCTGTACCTGCATTTGACTCTCATTGACCTGTAGTTCCCAGAGCCCTTCTTGCTCATAAG
CCTGAGATAATGCCTCTCATGTTTAAAAACATGGTTGTATAAGGAATCTTGTTTCTCATTGGAGGTCCAC
TTTCTGTTTTGTTTTGTTTCAGACATTTATGGTGCTGAACAAAGGGAGGACCATTTCCCGGTTTAGTGCC
ACTCGGGCCCTGTGGCTATTCAGTCCTTTCAACCTGATCAGAAGAACGGCCATCAAAGTGTCTGTCCACT
CATATCCTTTGCACGATTGCCTCTTCCACGTGTTACAATTAGACCTTGCCTACTGTGTCCAGTACCTTCT
TTAGAGGTTAGCAACAGAGCCCTTATCCAATTGAATCCCAAAAGTACAATGAGGTCATTAGATTGAAAAA
ATGGCTTGGGCCTTTAATGTGTCCCACACCCCAAAGCAGTCCCAAGACACAGAAGAAGTGTTTATGCAAC
ACACAGGTGGTTTGGATTCACCTCAGACTAATCTAGACACACCTGTGGATGCACAACTTGAAAAACATCC
TCTTTATGAGGGGAGGTTTGCTGATGCCCCCAGACACATTCTCACCACGGTGTGTGGTCCGCTTTCCCCA
CAACACAACAGGCAGTAAGAGGGCCCAAGCCTTCTTTGGGCAGATGAGATAGGCATGAGTTGTTGCTCTG
TGCCATAGCCATGCTGAGAAGTGGCTGCTTACTGGGCAAAAGAGCAGCACCTCCCAGAGGGCAAGGAAAA
CAGACCATGATGGGAAGAGCACACATAGTCTCAATGGGACCTCATTGGTCATGGGGCACTGTTAACATCA
CAAATTACTCAAAAACTACTAGCAGGTTTAAAATGTCAATAATCTGATGTTCATGCCATACAAGACTGTA
ATGATTAGAGACACATTGGTCAAATGTCTACTATATGAATTGTTTTAGGTAATGTCAGAAGCTGCTTTCA
GGGGATTTTGCAAGGCAAGCCAAACTAGTAGAAAAAAGATACTTTGGTTTGGTTTCCTAAGATCTTCCTG
GGTGATCCTAATGGAAAGATCCTAATGACCTAAGATCCTTGGTCATCGGAAGGATTGGAAGTTCTTTATG
ACAAATTTAGCCCTGGCTTGCAGGAGGTTGATGTACAAGAATACACCTGCCTCAGTTGCCTGACAAATGG
TAACCAGCCTTTTCTTAGAAGGCTTCAGTAAAAAAGAGTTCTACCCTGCTCCCTAACTCTGAGGCAGATT
GTTGGATTTCTGGACAGTTGTCATTGAGCTGGTAACCTTGGCACAGTCATCTATCTGCACACTCAACATG
AAGCTGCACAGCTTGATGAAGTTGCATTACGTTTTCTCTTTTTACATTTTTTTCTTTCTGGCTGGCCAGC
AAGAATTACGTTTTCTGAGCTGAATCTTGAATTTTCAGATTCTACAGAAGGATATTTAATGACAATACAG
TCTCTCACTTGCTAAATACTTCCTAAGTGTCAATACCATCATCAGTTCAAAAGCCTCTGAAGTAGAGATT
ACCATCCCCATTTTACAGATAATGAAACTGACACACAGAGGCATTAAGTAAATTATCCAAGACCATGTAG
CTAGTTAAGAAGTCCAGCCTGCATCCTGCGTAGAGCAGTATCTACAGTGAGGAAGTAGGGTGGAAAGTAA
TCAGGGTAATTATTCTCTCAATGTGTCGTCTCTGCTTGGGTAGTTGTTGCTTTTTCAAAATATATACAAT
TACATGGAAATTACAAATAAAGAGAAATCACAAACATACAAACTCCTCTTTATGGATTCTGGTTGAATTA
CATCTGGATAAGTAAGAAACAGCTAATTAGAAATAAAATCCCCTAAAAAGAACCAAGTGACCAGTGAATA
TTCTCTTCATCTTGAGATTTGATGAGTAAATAATTGCTTGGTAGAGTCACAGCTGGACAGAAAAGCTGTT
GAGGGGGTCTCTTCCTCACAAAAGTCTGCATTTAAATCTCATAAATTTGGTAACTCCAGCCCCAAAAGAT
ACTAGAATTCTCAGGGCCCTATTATTAATATTTCACCAAAAATATAATTCTAATCACATTTGCTATGAAT
GTTTTCATAACTAATATTATACATAAGATATATTACTTTTAAATATTTGTAGAATTGGAGACACGATTTT
AGTTTAATCCTTGGATTAACATTTCCCCCCATGTGAGCATATTTCTTAATTTAATTGTAAGTCAGCAGGA
AAATGTGGTTTAATTAATAGAATTTTCCTTTTCATAATAGATGTGTTCCTAAGTAACTGTAAAACTAGAC
ACATCAAGGCATTTACAGAAAACTAAAATACATTTAAATACTTCTGCTCAAATTGAAAAATGAGTGTTGT
ACTCTCTCTGTAGGTCAAGTTTATCTTCAAGTTTTATTTTTCCTTAGGAAGCACTGTTGGGAGCCCAGCT
ATTCCTAAGAAAAATGTCCAAGTGGGAACAATTTACCTTTAGATTCTCCCCACCAACATTTCTTCCCTTC
TATTTTCCCCTTCCACCCACCTTCCTTTCAATGTCTCCCTCCCCTTTTGATAATCCCTTTGCCCATAGTC
TGCTGTCAATATTCTCCACTCTAGCTATTCAGAGTTCTCCACTCCAGCCTAATCCTACTTGGGGTTTTCT
TCCTGAGCCAGTATTTCATTTCCCTGTGCCTGCCTCCACCGTTCCTACCCAGAAGACCATTTCCTCGGTG
GAACTGATCTCAACTAAGTTACCCAAACAATCTGATGAAACTAATTCCCATGAAAGTTTGTACCTCTTCA
CTTATCTCCAAGTTTCTAACTTTTCTTTGTCTTTTAGAATAAAAAAGACATTTGCCAGATGAAATTTCAG
GCCATTTTGTATACTGAAATGAAGGCTTCGGGGCAGAAGGACATTTTGGAAAAGGGAGTCTGTTTCCTTC
TATCTTGCTCAGAGTTTGTTATCAAACAGATAAGAGTTCACATCTCTGCAGCATGGTTGACTAGCTGCAT
GACCTGAGAAAAGTTATTTAACTTTTATTTTTTGTCAGCCTGTTTCTCAGCTTCAAAATGTAGATATTTA
TACCCACCTGTAGATCAGTTGTATTAGAAGTAACATGGACAAAGTACTCAGCCTAGCTTAGGCACTCAGT
AAATGTTAGTGGTTATTATGTGACAAGATCATAAAGCTAATATTGGCTAAGATTTATGAATGTAATCATT
CAGCATCAAGGTGATCCTAGATGACATCACAGAGACTTGGTGATCTCAGCTTTTCCTTAACACAGGCCTT
TCAGGTGGTTCAGTTTATTTATTACGGTCACTATTTTGGTTAATTGTGTGTGCATGACCCGAACTGACCT
TCCAGAGAAAATTGAGTGAGTAGATTTGCTATCATTATTGTACCATCTCTTTTTGCTTTCTTTTCTTCCT
TCACTTGGCAGTCTTTGTCTTTTATTTATCCTGCCATGCCTCATGTGGGTAATGCTGAATCTCATTGTCT
ATATGTCTTTGTAATAACCAGGTCTCTCCTTCTGTGAGATTTGTTTCATAAACATACATGATTTTCAGTT
TCAAAGAGATACAGATTAGTGGTTCTCAACTTTGGCTGCATATTGCAATTTTTGGGGAGATTTTTTAAAT
GCCTGAGTCCCAGCCCCAGAAAGTCTAAGGTGGCCTGGGAAGATGAGTCTCCTCGCCCCCACCCCCCCCC
AACACACACACACTCACTCACCTGCTAGATGCACACACAAATGTATGCCTTAGAGAAGGTATGCTTTTGA
GAAGGCCTGCTATGAAAGAGAGACAGGAGCAAAACTGAGACAGAACTAAGAACAAATGGCTCTGGAGTAT
ATTTAGAAAATAACTCATAAATGAAGCTGTTTAAGTTATCATGCTTCATATGCCTCAAAGTAACCTTAGT
ATTCCTGAAGTTATTCTATTTCCCTAGGTTAGTGGGTGAAACCCCTATAGTTTCAGAATCTGGGAGTTTT
CCAAAAACATTCCATATCCACACATCTGGGAGGGATGCTTCCACTCCAGCTTGAACCTAACATACCTTTG
GTTATAATAACAGGTGCTTTGCAGAGCTTTCTTTTTTTTTTTTTTTTTTTTTTTGCTAATCATAACAGTA
ATGCATGCCACAACAGAAAATACGTTATATTTTTATTAATAGATTAAAGGAAAAGGATCACATAGTCTTC
TTAATTGATGCTAAAATCATTTGATAAAGTTTAATTTCCATTTATAATTTTAAATTCTTAGAAAAGTTGA
AATAGAAGGGAATAAATGTGGCACATATACACCATGGAATGCTATGCAGTCATAAAAAAGGATGAGTTCA
TGTCCTTTGCAGGGACATGGATGAAGCTGGAAACCATCATTCTCAGCAAACTAACACAAGAACAGAAAAC
CAAACACCGTATGTTCTCACTCATAAGTGGGAGTTGAACAATGAGAACACATGGACACAGGGAGGGGAAC
ACACTGGGGCCTGTTGAGGGGTGGGGGGCTAGGGGAGGGATAGCATTAGGAGAAATACCTAATGTAAATG
ACGGGTTGAAGGGTGCAGCAAACCACCATGGCACGGGTACACCTATGTAACAAACCGGCACGTTCTGCAC
ATGTACCCCAGAACTTAAAGTATAATAATTTTAAAAAGGGGATTGCATCACTCTGATAAAGGGTATTCAT
TGTTTTAAAGTATCAAGCGTCATACTTAATTGTGAACATGGAAAGCTTTCCCTTTGAGATCAAAAGAAAA
AAAAACCAAAGATGTCTGTTCTTCTCAGTTCCACTTTGTAAAGGAGGTCTCAGCCAGCATAGTAAAGCAA
AGAAAAAAAAAGTGCAAGAAGAAAAAAGTACCGAAGATGTAATTTACAGATAATAAGATTGTATACATAA
AATATCCAACATAACCTAGAGATAAACAGTAAGAATTAATAAAAGAGTATAGTAAGGTAGCTGGGTACAA
ATTAACATACATAAATCAATTGTGTATAGACACTAGCAACAAACAGAAAATAGTTTTTAAAAATACCATT
TACACTGGCATTTTAAAATGTCAAGTATCTAGGAATAAGATTATCAAAGTATAAGTGAGATCTCTATAAG
GAAAATTATAATAAGTATTGTGTAAAGTTTTTAAAGAAAACCAAATCAGTGGGGAGACATGTTCTCAGAT
TCAAAGTCTCAATATTGTAAAGACTCCCAAAAATGATTAATAAATTTAATATGATCCTCAAATCAATATT
TCAACAGGCTTCTTTTGGTATTACTTGACAAACTTTAAACTTTAAATTTTCTTCCTCAAAAGTTTACATG
AAATTTTAAAGAGCAAATTATAGCCAAGAAACTTTTGAGGAAGAAATACAAAGTGGGAGAACTCATTCTA
CTGGATGTCGAGATTTACTATAAAGCTCTAATTGAAGCAGTGTGCCATTGGTGTAAGATTGGTCAGAATA
ATGTAACAGCATAGACAGTCCATAAACAGACCAACACATATGTAAACATCTAATGTGTGAAAAGGTGGCA
CCCCAGAACAGCAGGGAAAAGGCGGTCTTCAAAAAATGATGCTGAAGAATTAGGAATCTCACTGGAAAAA
CTACATTAAACATCCAACTTTGCACCACACACAAGAATCAATTCTAGGTGGATCAACACCTCACTTGCTT
TGTGAAATGCAAATATATAAAGCCCTTTTTTAAATTTTACTTTAAGTTCTGGGGTACATGTGCAGAAAGT
GCAGGTTTGTTACATAGGTATACACATGTCATGGTGGTTTGCTGCACCCATCAACCCATCATCTACATTA
GGTATTTCTCCTAATGCTATCCCTCCCCTAGCCCCCAACCCCTCAACAGGCCCTGGTGTGTGATGTCCCC
CTCCCTGTATCCACGTGCTCTCATTGTTCATCTCCCACCTATGAGTGAGAACATGCGGTGTTTGGTTTTC
TGTTCTTATGTCAGTTTGCTGAGAATGATGGTTTCCAGCTTCATCCATGTCCCTGCAAAGAACATGAACT
CACCCTTTTTTATGGCTGCATAGTATTCCATGGTGTATATGTGCCATATTTTCTTTATCCAGTCTATCAC
TGATGGGCGTTTGGGTTAGTTCCAAGTCTTTGCTATTGTCAATAGTGCCACAATAAACATACGCATGCTT
GTATCTTTATAGTAGAATAATTTATAATCCTTTGGGTATATACCCAGTAATGGAATTGCTGGGTCAAATG
GTGTTTCTGGTTCTAGATCCTTGAGGAATCACCACACTGTCTTCCACAATGGTTGAACTAATTTACACTC
CCACCAACAGTGTAAAAGCATTCCTATCTCTCCACATCCTCTCCAGCATCTGTTGTTTCCTGACTTTTTA
ATGATCTCCATTCTAACTGGTATGAGACTCCATCTCATTGTGGTTTTGACTGGCATTTCTCTAATGACCA
GTGATGATGAGCTTTTTATCATATGTTTATTGGCTGCATAAATGTCTTCTTTTGAGAAGTGTCTGTTCAT
ATCCTTTGCCCACTTTTTGATGGAGTTGTTTTTTCTTGTAAATTTGTTTAAGTTTTTTGTAGATTCTGGA
TATTAGCCCTTTGTCAGATTGATAGATTGCAAAAATTTTCTCCCATTCTGTAGGTTACCTATTCACTCTG
ATGATAGTTTCTTTTGCTGTGCAGAAGCTCTTTAGTTTAATTGGATTCCATTTGTCAATTTTGGCTTTTG
TCACCATTGCTTTTGATGTGTTAGTCATAAAGCCTTTGCCCATGCCTATGTCCTGAAGGGTATTGCCTAG
GTTTTCTTCTAGGGTTTTTATGGTTTTAGGTCTTATGTTTAAGTCTTTAATCTATCTTGAGTTAATTTTT
GTGTAAGGTATAAGGAAGGGATCTAGTTTCAATTTTCTGCCTAAGGCTAGCCAGTTTTCCCCACACCATT
TATTAAATAGGGAATCCTTTCCCCATTGCTTGCTTTTGTCAAGTTTGTCAAAGATCAGATGGTTGTAGAT
ATGCGGTGTTACTTCTGAGGCCTCTGTTCTGTTCCATTGGTCTAGATATCTGTTTTGGTACCAGTACCTC
GCTGTTTTGGTTACTGTAGCTTTGTAGTATAGCTTGAAGTCAGGTAGCGTGATGCCTCCAACTTTCTTCT
TTTTGCTTAGAATTGTCTTGACTATGCGGGCTCTTTTTTGTTCCACATGAAATTTAAAGTAGATTTTTCT
AATTCTGTGAAGAAAGTCAGTGGTAGCTTGATGGGGATAACATTGAATCTATAAATTACTTTGGGCAGTA
TGGCCATTTTCACAATATTGATTTTTCCTATCCATGAGCATGGAATGTTTTTCCATTTATTTGTGTCCTC
TCTTATTTCCTTGAGCAGTGATTTTTAGTTCTCCTTGAAGAGGTCCTTCACATCCCTTGTAAGTTGTATT
TCTAGATATTTTATTCTCTTTGTTGCAATTGTGAATGGGAGTTCACTCATGATTTGGCTCTCGGTTTGTC
TGTTATTGGTGTATGGGAATGCTTGTGATTTTTGCACATTGATTTTGTATCCTGAGACTTTGCTGAAGTT
GCTTATCAGCTTAAGGAGATTTTGGGCTGAGATGATGGGGTTTTCTAAATATACAGTCATGTCATCTGCA
AACAGAGACAATTTGACTTCCTCTTTTCCTATTTGAATACCCCTTATTTCTTTCTCTTGCCTGATCGCCC
TGGCCAGAACTTCCAATACTATGTTGAATAAGAGTGGTGAGATAGGGCATTCTTGTCTTGTGCTGGTTTT
CAAAGGAAATGCTTCCAGTTTTTGCCCATTCAGTATGATATTGGCTGGGGGCTTGTCATAAATAGTTCTT
ATTATTTTGAGATACATTCCATCAATACCTAGTTTATTGAGAGCTTTTAGCATGAATGGGTGTTGAATTT
TGTCGAAGACCTTTTCTGCATCTATTGAGATAATCACGTGGTTTTTGTCATTGGTTCTGTTTGTGTGATG
GATTACGTTTATTGACTTGCGTATGTTGAACCAGCTTTGATCCCAGGTATGAAGCCGACTTGATCGTGGT
GGATAAGCTTTTTGAAAGGAAAAAACAAAGAGGAATATCTTTATGACCTCAGACTAAGAAAAACTTTATT
ACATATGACACAAGAAGCACTAACTATACAGGAAAAAACTGATACATTTTAGTATACTATAATTAAGAAT
GTTCATCAGAAGACAACTTTAAGAGCATGAAAAAGCAAGTGCTTATATTCAGAGTAATGAACTCTACAAA
TCAATTAGAAAAATGTCAGAACAACCACTTGTAAAATAGCACAAAATTTGAATAGGCACTTTACAAAAGT
GGAAATCCAAATGGCCATAAACATATGAAAGTATTCTTAACCTTTTTTTAAATCAAAGAGTTGCAAATTA
AAATCACACTGAGATTTTATTACATGTTCATCATAGAAGGGAAATTTTTTAAAGTGTCACAATACCAAGT
ATTGGGAATGACATGGAGCAACAGAAACTCACACACTGCTGGTGGGAGTGTAAATTTGGACAACCTCTTT
GGAAAGCAAGGGGTATTATCTAGTAAAGGTGTAAGCATACATATGCTGTATTCTTCTATTACACCCTTAG
GTACCTATGTTTAACAGAAATATGTACTTGTGGACACCAGATTACAGCTACAAGAAAGTTTATGAACATT
TGCCCATAAAAACCCCAAACCGGAAACCACTCAAATGTCCATAAACTTTGAGTACATTAACTATTTCTGT
TATATTCTAAAATGAGAAAACTTACAGCAATAAAAATTAATAAACTTTAGTGCTGCACAACAATAAGGAT
GAGTCTTAAGAAAGTAATATTGAACAAAAAAAGCCAAATGCAAAGGATTACATAAACAAGGTTCATTTAT
ATAAAGTTTGAAATCAAGCAAAACTAAACTATTTTGTTTAGGAATGCATACTTATGAAGTAAAAAGCATC
AATAAAGTAAGGCAAGAAAAAGTACCAAAAATTAGGAGAATAGTGGCCTCCAGGGCAGAAGAGGGAGGAT
GTAATTGGGAAGGAGCTGGTGAAGTGCTGGTCACGTTCCATTTTATGGCATGGGTAGTTGTCACACAAGC
TGCTATGGAGTGAATTTTGTCCCCTCAAAATTCAAATGTTGAAACAAACCCCCCATGTTGCTATGTTGGA
GATTGGGCCTCTAAGGAGGTGATTGGGTTAAATGAAGTCACAAGAGTAGAGCCCTTAAGTGGTAGAACTG
GTGTCCTTATAAGAGAAAGAGACAGCTTTCTCCCCTTTGTGTGAGAACACAAGGACAAGGTAGACATCTA
TAAGCTAGAAAGAGAGTCTTTCCCAGACCCAACTATGCTGACATCCCAATCTCAGGCTTCAACCTCCAGA
TTTGTGAGAAAATAAATTTCTGTTGCATAAGCCACCCAGTCTATGGTATTTTGTTAAGGCAACAAAGGCT
GACTAATACACAAACATTTGTTTTAAAATAATTAATTTATGTTTTATGTACATTTTTATTATGCATGTTA
CATATCATAATAAAAACCCAATAATTTTTTAAAATAAATGACCATTGCAAAAAATGCAGAACACTGAAAA
TATAAATCAGAAGATGGTTACTAAGTAGAGAAATCACAAAATCATGAAATTATATTTAATATTGTTGAGA
TCATATAGCATGTAAAATGTCTATGCTTCTTTTGGAGTTTTAATGTAAAACATTTTTTTGCAAAATGAGT
TATTTGGGAGTTATTTGGAGATTGTGGTTAAATATGGAAGATAGAATGCATATGTTTTACCCCACCCTCT
CAAAATGCCAATAGAATGACAGCACGGGGGAAAGAACAGTATAAATATACAAGAACAAAGAGAAAGAGAA
TGGAAAACACAGAGGACAAATGATGTCAAAACACTTGGAAGACCAAAAGTGGGTGGAGAAGTGGTAAATC
ACTTAGCAGATCAGGGTTTGTTAAACCACGGTGCCTCCAGAGGGGGATAAAAATGAGAATAAAGGCATTT
TACCGCCCCCGCTGCCCAACAAGCCCCAGAGGCCCTGCAAGTGTTGACATGATACAAATAGCAAGAAATG
GAAGCAGAAAGAGGAAAGAACATGTGAGCAGAGTATGTTCACTTACTGCTGCTTCTATAATAACAAGAAG
TTAAAGAGTATTATTTAAAGTTGACAAATAAAGAAATGGACAAAATAATTACACTTGACAGTACAAAGGT
AAGCACAAAAAGATATTATTAAATACAGTCAGATAATGGATAAAACAGAGAGGAAAGGAATCACCAAATG
GAAATGAGAGATTTTGATTAAAGAAATAGTACACTAAGGACAATGTATTACACGGTTTTAATTATAATTA
TAAATTTATAATAAGTTTTTACTTATAAAGACAACCACTAGGATAAAGATACAAACTCTTTAGTAATCTA
TATATTTGTAACAAATAATATGACAAACTCTAAATACTGGAAGAATTGTACATCAAAAAATAAAAAGCAA
ATAAGAGATGCCAAATAATGAAAAGGTACAAAAGTAATTGGGGTTTCAGACCATAAATATTAAATCATTA
TAACTAGGCTCAAACACATCTTTATTAATCAAAGTAGGAACCATTACAATCAACACATTTTTGCCAATGA
GAAATGAGTTTGTTTATTCCTGTAGCATAAAAATCCATGCTTTGGGATTCAATGAACTCTTGGAAGGCAT
TTTCTGCATCCCGCTGGTTATGGAAGTGTTTCACCTGCAAAAAGTTGTTCAGATGCTTGAAGAAGTGGTA
GTTGGTTGGTGAGAGGTCAGGTGAATATGGTGGATGAGGCAAAACTTCATAGCCCAATTCATTCAACTTT
TGAAGTAACTGTTGGTTGTGTGACATGCAGTCGGTTGTTGTGGAGAAGAATTGGGCCCTTTCTGTTGACC
AATGCCAGCTGCAGGCCTTGCAGTTTTCTGTGCATCTCATCGATTTGCTGAACATATTTCTCAGATGTAA
TGGCTTTGCTGGGATTCAGAAAGCTGTAGTAGATCAGACCGGCAGCAGACCACCAAGCAGTGACCACGAC
TTTTTTTGGTGCAAGTTGGGTTTTGGGAAGTGCTTTGGAGCTTCTTCTCAGTCCAGCCACCAAGCTGGTC
ATTGCCAGTTGTCAAGTAAAATTCACTTTTTATCGCACATCACAATCTGATTAAGAAATAGTTCATTTTT
GTTGCATAGAATAAGAGACGACAACACTTCAAAATGACGATTTTTTAAACATTTTTCCTTAGCTTATGAG
GCACCCATTTATGGAGCTGTTTCACCTTTCCAATTTGCTTCAACTGCCGAATGACCGTAGAATGGTCAAC
GATGAGTTCTTCCTCAGCTTCTTGTGCAGTTGTAAGAGGATCAACTTCGATGATTGCTCTCAATTGGTCA
TTGTCAACTTCCAATGGCCAGCCACTACTCTCCTCATCTTCAAGGTTCTCATCTCCTTTGCAAAACTTCT
TGAACCACCACTGCACTGTACCTTTGTTAGCAATCCCTGGGCCAAATGCGTTGTTGATGTTGAGTTGTCT
CTGCTGCTTTACAACCCATTTTGAACTCAAATAAGAAAATCGCTCGAATTTGCTTTTTGTATAACATAAT
TTCCATAGTCTAAAATAAATATAAAATAAACAGCAAGTAATAAGTCATTAGCAAAAAAAAAAGTGAGAAA
TGCACATTAAAATGATGTATAACATGGTCACATTTATTTAAGAATGTATTCCAATATCAAACGGCAAATT
TCAACAATGCAAAAACCACAATTACTTTTGTACCAACCTAATATTTTAAAAACTAAATACGTCATATGAA
ATAATATAAACAAGCTAAGACTAAGCAAATCTGTCATGTCACTAAATATAAATGGATTTTACTCACCTAT
TTTTAAAAATGCCTATTACATTACAAAGCAATACCCAACTCTATGTTGAATAAAAAAAATACTCCTCACG
GTGATACAGAAAGGCTAAAATTATAGAATTCAAAAGACATAAGCAAAGGCAAACAAAGAAAGCAGGATCC
ATGATTTTAATATCAGAGAAAGTGGAATCTCGGTCAAAAAGCATGAAATAGGACAAAAATTGCACAGACA
TATTAATATTAAAGACGATAGTAATCTATATCTTTGCAACAAATAAGATGGCAACAATATTTATAAATCA
GAAATTATTAGAGTAGGAGGATAAATAAAAACATTGTAGGAATAGAAATTTTCAATTAATTTTAGCCAAT
AACAGATCAAATGTATTAAAAAGTATGTAAAATTATAGATGACCTAGTAATATAATTAATATAATGTATC
TGATTAATATATAGCAAACTACCTTCCCTAACAATAGAAAATGCACCTCCTTTTCAAAAAACCAAACATA
TCAAACCAAATATTAAGCCATGACAAAAACCTCAATAAGCTCCAGAAAGTGGAAATAATATAGAAAACAT
TCTCTGATCATAATGAAATAGAGTTAAAAAAAATAACCAAATTTAATGAAATGAGAGGACTTTCCACTTG
ATAATTTTTAAAAATCTCTCTTAAAGAATTCTTGGACAAAAGACAAACTATAAACTAAAATTATATAATT
TCGAGGGAAGGAAAGATAAAAAACAGTTTAATCAGAACCTAGAGAGTGCAGTTAAAGACATACATATTCA
GGGAATAAGTCACAGCATGGAAGCATAGAATATTTATATCAGTAAAAGCTGAAAAGAAATTATTTAAAGA
CCTAACAAAACTAGAAAAAGGATAACAAATGAAAGAAAGAAGTTAATAATTATAAAAAGCAATAAATTAC
AAAATAGAAAAACAGTAGATCTAATAAATCCAAATGCTGTTGTAAAAATATGCACAAGACCTTTATACTA
AAAAATAATACAGCATTATTGAGAGAAATTTTAAAGTACCTAAATGAATTAAAGGATGTCTCATGTGACT
GGATTAGAAGATTCATAATTGTTAGAATGTCAGTGCTGCCCAAATTAGTCTATAAATTCAATGACATCCC
CAAAAAAATCCCAGCAGCAGCTGTTTTATAGAAATTAGCTGAACAGATTTCATTTGCATGGATGAAGTGA
AGTTTATTTAACTATTTTCCTATTGATAGACATTCACATTTATCATAATTAACATAATCCTCATATTTTG
CCACCACATATTAATTTTTTGGTGAGTATCCTTACACATGTATCTTTGTACACATAATATGAGTAAAATT
AAGGATGAATTTCTAGAAGTAGAATTGCCAGGTCAAAGGGGTGTACATTTTAATTTTTGAGATATTGCCA
AATCGTCTTCCAAAGTTGGATCCTTTCCATTCCCAGTAATTATCAAATCCATTCTTTAGGAGATTTTGTA
TTAACTGAACTGTTGACATAATTCCCTTTATATGCACACTATGAAAAGTATAACATATACAAATCTACCT
ATATGGTCACAATCCTGGGAATTTATGTGACAGGTATTGGATGATGTTCAGCATCCTTGGCCTCGTGGAA
ATAATCAGACTAGATTTTCTTTTTGAAATGCAAATTGATAATAGCCATTAGTTGTTTCCTATGTTGCTGA
GAACCCAGAGGAAGATAATAAATGTCCTTGGGATAGACTGGGGCTCCAAAGATCATGCTGTATACTTTTG
AAAAAGCATTAAATTGTAGCCAGTGAGGAATCAGAAACCAATCTTATTGCCATGTTCTTAACACAATCTT
TTTCCTTATCTTCCCTCCCTTCCATTATTCCTTGCTGAAACATTAGCTTTGCACAATTACCTGCTGATAA
TGGTAGCTCTATGTCTTTCTGCGTGCCTGCCTATCTGTCTGCTTGTGGAAAGATAAAGATAGATATTCCA
TTTTCTAATCCCCTCTTTTGCTTGGGAAGAAACTAGAGGTTCTTCCTCAGATTGGGCACCAGAAGGTAAA
GAGAGCAGGGGGTCAAAAATTGTTAAGAAGGGCAAGACTAGAACTTTCCTACTCTCCCCATGCCCTGGTT
TCAGAGAATCATTGAAGGGGCTCTTTGATGACCCTAGGACACTCACCTCTAAGCATAACAATATTCTGGA
AAACCTTTGACAAATTATAGCAATTTGGTTGTAGAGAAGATAAATGTTTTCTCACCTGGCAAAGAACATG
TGAAATGAAGCTGAATCTACTGCAAACGGGGAAGTGGGTGTTTAACACTGCATGCATAAAGTATTGATTT
GTATCTTTTCAAATTTATGGAACTGTTATCATTTTATATGTGTGCTTTATCTGATGTTTTAAAATTATGT
TTTTGGGATTTGCCCAAATGGATACATGTAGACCTAATTCATTCATTTTTTATTGCCATATAGTATTCCA
TAAAGATATTATCCTGTGTTTTCTCCTCAAAGTTACAGTTTTCCTTTCTACATTTAGGTATTTCAATCTC
CTAGAATTTATTTTTGTATGTGTGTGGTATGAGATAGGCTTCCAATATTTTTCCATATGGATTATTCCAA
CATTTTTTTCAATACATCATTTCCCAAGTGGTTCCTACCACTGCTTCTGCCATGCACCAGATTCCCATGT
CTACTGAATCTGTTTCTCCCAGTAATCTCATTGTCACATCCTGAGTCAACACCACGTGGATTTAAATGCT
CTATAGTAAGATTTGACCTGGTCGATTCACATGCCCCTCCTTATTTGTCTTCTTGAAAATTATTTTGGTT
CTTGTTTCTTTACTTTTCCATATCAGTTTAAGGGTCACATTTTCCAGCTCTTTTCTTACAACTCTGTGGA
AACTTTGTTTGGTAATGCACTGAATTGATTTATCACCACAGGAAGAATCGCCACATTTACAATACGAAGC
CTTCCCACACATCAGTATGGCAGATCTCTTAATTTATACAGGTCTTCTTTAATATTTTTCTACATTTTAT
AATGTCTTTTTCCTAGGTACATTGCAATTTTGGTTGCCCTATTGAATACTTATTATTGCTAAACTGTCTT
AATATTATTAATTTCTATATAGTGATTTTGCATCCAGCAACCCTGCTGAGCTCCTTAAAGTTAAATTTTT
CTGTAGATTCCCTTGGACTTCCTATTTAGACAGTTATATTATCTGCAAATAATGACAAACTTGTTTCTTC
CTTTATGATTCTTATACCACTTTTTTATTTTTCCTGTCTTATTGAATTGGCCGGGACCTTCAACAAGATG
GTAATTCAAAGCTATGACAGTGAGCATATTTATTTTGCTCTTGATTTTAAAGTGTGTGCTTCTAATGTTT
CACTATTATGATGTTTATGAGAGTCTCATAATTACTTTTTATATCAATTTTTTCCATTTCTTGTTTGTTG
AGACTTTCATCATGAATGGGTGTTGGCTTCTATCAGTATTTTTATATTTATTGAGGTTATCATATAGTCT
TTCATCTGTTAGTATAGTAAATCTGTAAACTAACAGGTTTGTTAGTCTATTTTTAATGGTTCCAGCTGAT
CATTTTGCTGGATTCAATTAGGTGTATTTCATTTTATCTATTTTGCTAAATGAGGCTGGCCTGTAATCTT
CTTTCATAAACTGTCCCTTTGGGATGTTCCAACTCTCTCCTCAATTGTCCCTGGGAACTCCAACTTATTC
TTACTTTGTTTAGCAGAGCAATTCATCTCTCTCCATCTCCTACAATGTTTCTCAACAAGTGATCTAAGGA
TCTCTATGCACATATAATTTTAAAATCTAACATGTAAGATTAATTATTATATTCATAGAATTTCTAACTT
TTTATGTTTTCAAAAACAGTGTATGTGCTTCAAAAACTGATTTCATTCATAATGGTTAACATGGAGTTAA
TCATAGGCAGTTTATCTTGACTAATGTATTTTATTGTCACCTGACAGTTATTTACACTTGTCAGACTTGC
ATTTGCCATTATTACTGTCGCTTTGCATAATATCACAATTCAAAAAAAAGAGTCATCACTTCGAAAGTGG
TTTTCTGAAGCATAACAATAATGATAAAAATAGAAATATAAATCCTACAATGTGAAATAAGGTCACAATG
TAAGTAATGAATGGCCTCCATTGTAAAAATACAAAGCATGAATCTGTTTGTCATGAGACAAGTGGATAAT
GGATGTGGATAATCTCCCAAAACCAGACAAAAATATATAAGAAAATGGGAGCCATGACAATGAATAATGA
TAATTGAATTTTATTGGACCAATGATCCTTTTGTCCTAGTACCCAATTTATTATCTGCTAACGTTTTTGT
CAAGTAATGATATAAAGCTATCAAAACTTCCATATTATTTTCCAATAAAGTTTATCAACCCCACTTTTAA
ACCAATTGAATATCTCTACTACAAGCACAAAATAAGGCTTTCCCAGTATAAAATTGTGAAAATTTTATGT
TAAAGGTTGAGAAGACAACAAAACTATAGAGTCATCATTCATCCTCATATCAAAAATAAGCACAATTGCA
CTCCTGCTGGTAAGATACAAAACCAGTTTCCAAATCAATTACAAATATTGTGGTTGGAGAAGAAACAAGA
AAAACAATTGGTATAATTCCTTAATTAGAAGTATTGCATTCCTTATAATGTGACTGGCATTTAAAGTCAA
TGACTGTTAGGTATACGAGCAGACATTTTATTTTATAGTTGAACAAAACTATGAATAAGTAAGTTGTCAG
CATATCTTCACTATATATATGGGAAAGACTCAGTGACTTTTTGTTCTATTTATCATGAAGCTAATTTTAA
AAGAAGAGCTCTCTGGTTTAATTAAAAATTAGTTTGAACCTATGAAACAGACTTTAAAAGGTATGTTGGC
TTATGCACTAATGTTTGTAGCAGCGTTATTCACAATAGCCAAATATGTCCATCAACAGATGAATGGATAA
AAAATGTAGTATATATGTAAACAATTGAATATCATTCAGTTTTAAAAATGAATGAGATTCTGATACATGC
CACAACATTGACAAAGCATTGTGCTAAGTAAAATAACTCAAACGCAAAAGAACAAATGTTGCATGATTCC
ACCTTTAGTAGGTACCTAGAACAGGCAAATTCATGGAGACAGAAAGTAGAATGGAAGTTGCCAGAGAGGG
GGAATGGGGAGTTATTATTTAATGGGTACATTTCTGTTCGGGATTATAAAAAAGTTTGGAAACAGATAGT
GGTGATAGTTCCACAATACTGAATGTACTTAATGCCACTAAATTGTGTACTTAAAAATGGTGAAAGTGGT
AAATTTTATGTTATGTATATTATGCATATTTTACCAAAACTTTTTTTTAAAAGAGGAAAGGTGTTAAATT
GCCATTGAAGAAACTCCAGCCACATGGTCAGCAAGGAAAGGTATCACTATCCAGATAGAAGATGCTGCTC
CCAGATCCCAATGCTAGTGCATACCTTATTCACGGAACGCAGCTGGGGGTCTGTAATATGCCTCCAGATT
CTGATTTGCTGTTGAGGTACACAGTGGAAATTGTGAATACAAACTGATTGTGGTCACTTAGTGTACATCT
CTTTGGCCTACTGGGCTGAAATGAAAAGTGAGTAAGAGAGGCTGCTCTCCCACACTGAATTGCTTTGGCT
GGCAAATGGGAAGGGCTTACACAGAGTTTTAAAAATAAGGAATGAAATGTTTTAAATTACCATTTTTATG
AATACAAGTTGGGTATCTCAGTGATCCATTCAGAATTTGAATATTATGAATCTTTGTTTTCAGAGCCTGA
ATATCGGGATTTGTAACAATCAGACATCAGTGGTGCAAGTGGCTCAACGGCTGAGAACTTGACTTTTTCC
CAATACCCAATGATTTTCTTCATTCACCAGAAAAAGCACTTAGGAAATAACACATTGGGCTTATTAAAAA
TTACATCTGAATGCTGCAACCCAACATCAAGAAATACCTTGCAGAGCCAAATACCACCAAAGAGAGGATG
CAACTCCATTAGCTGCTGTTGAGCTGGGGCACTTGCCTTCTGGTGAAGCACTGAAAATAGTCTTTAGAGA
GCACTCTCTCCATAATTTCTGGGTCTGTGTTTGATTTGACAAACCAGAAATATCCATCAAAGCTTCCCAA
CAACCTATAATTCTGAGTTAAGATTTTCTAATTTAGAAAACATAAAGAGTGTGGGGGGGAAACCAACAAG
ATATGGAAGCTGACAGCTCAAGCAGCTATGCAACCAGATATAGATTCCTAAATGATTTCAAACACGATTT
CTCCTCTCACAGATGTGAAAAAAAATTATTCACTTTTTAACTTGACTTTTACTTTTATTTAAGACATATA
TGTTCATAATTTAAAAGGTCACAGAGTTTTATAAAGATTCTGACAAAAAAAACAGCAATTTCATTTTTCT
CATCTCTTCTTTCCAGAGGCCACCACTTTTAGCTTTTTTTACCTGTGTCTTCTGATACCTCCCTATCTCC
AATAATATACTCATAATACTAATTTTTAATTTTTCAATTTCTGATGTTATTTATTGGCTTCCTATTATGA
GAAAAAACTTAAGGCCACATCCACAAACTCATTTCCTCTCACCCTCATCTTCCTGTTACAGTTATATTGT
AATCTTCAGTTAGATCATCAGATAATATATCCATTGTATGACTACATATATAATTACATTACATTGCTAA
GTAAACACTGTTTACAGCTGTGTCATGCAGTGTACTTTGACTATATTTCCTTTTTGCACAATGTTTTTGC
TTTCCTTGGAGTTAATAAGGATTTTATTTTTTTGTTTGCTCAGTTTTCTATTTATCCCTAATTTGTTCCC
AAACTCTTCTCCAGATGTTTAATTGTTGCTTCAATTTATTTGCACACATCAGGTGCATGTGTATCATTTT
TTTCCTATGGGAATCACAGCTAGAGTACTTCATCTTCTGCCTCATTCTGACCTAGATGTTTTCTAGGCCT
GATGCACAGCTGTTTTCTAGAGATTTTTTCTTCTTCATTCTTTTTTTGGATTCCTTTTGCCTCTTCCCTG
GATCGCATATATGTTTCTTGTTTGAATGGAGCATATCCTCTAGTAACATTTTTTTTTAAGACAAAGTCTC
TCTATGCTGCCCAGGCTGTTGGCCTCAAACTCCTGGGTTCAAGTGATCCTCCTGCCTCAACCTCCCAAGT
AGTTAGGACTACAGGTACATACCACCATGCCCAGCTTCTAGTAACTTTATTAGAAGGGTAACTAAGAAGT
ACATTTCCCAAGACTTCATATATTCTACCTTCTTTGACAGTTTGGCTAGGGAAAGACTTCTGTATTAGAA
ATTATTTCCCTCTTAATTTTGAAGGCCTTGCTCATTGTCTTCTAATTTCTTTCTAGACCTCCTATTAGTC
AAACAATATACCTCCTAGTCTGATTCTCTAATTTTCAAACTTTTTATCACTTCCTATCCATTTCTGCTTT
TTTGTTCCACTTCCTGTGAGATTTCCAAAACTTTATTTACAAGTCCGCCTATTAGTGTTTTACTTTTGCT
ATCATATTTTTGTTTTCAAAATTCCATTTTGTCTTTGAAGGTACTTTTTACATCCTTCTGCTCTTGTTCT
ATACGTGGAGCCTCTTCTCACCTCTTAACTCTCTAAAAATATTAGTACAGGTTTTTTTTTTTTTGTTTTT
GTTTTTGTTGTTGTTGTTGTTGCTGTTGTTGTTGTTGTTTTGGAGACGAAGTCTTGCTCTGTCACCCAGA
CTGGAGTGCAGTGACGCGATCTAGGCTCACTGCAAGCTCCGCCTCCCGGGTTCACGCCATTCTCCCGCCT
CAGCCTCCGGAGTAGCTGGAACTACAGGTGCCCGCCAAGGCGCCCGGCTAATTTTTTGTATTTTTAGTAG
AGACGGGGTTTCACCGCGTTAGCCAGGATGGTCTCGATCTCCTGACCTCGTAATCTGCCCGCCTCGGCCT
CCCAAAGTGCTGCGATTACAGGCGTGAGCCACAGCGCCTGGCCAATACAGTTTTATTTCTCTAACTCTTC
TTCTGCCCTCTTTATTGTCTCTTTTTCTTTTCTTTTCTTTTCTTTTTTTTTTTTTTTTCAGTTGTTTGGT
TGCTTTGGTTTCTGGTGTTAGAAGCATCCTTCAAATTCCTGATGCTCCTTGGCTGGCTGTTCATGTTAAA
AGTGCAGCATCTTCATGGGCTCTAAACCAGATTATCTTCTGGAAACTTAACGAGGAGCAGAAGTGAATAG
GGAGGGGAGTTGTTTGCCTTTCAGTAGGTAAACTTTTCTTTATATGCATTTTTCAGTATGCAACTGTTTC
CCTCTTCTTCAACTCTTAAGAAAATGAGCATCCAATCCATTTCTGGAGCAGAAACAGTTTCTCATTGAAT
GGATGGCATAAAGCAGCATCTAGAAAGCTCAACTGGTTCCTGTAAAAAACTTTCAAGCAATTCTTTCAGC
CCTACTTATCACCCTCCCCCAAACCTTGCTTTCAGAGATACTTAGCATCTCTAAGTTCCAAGCAGTTCTG
GGGCTCTGCAGCATAGCTTGGCTTGCTTCTGGGTATTCCCTGCTTCAGCCAGTTTTCAGCTTTCTTAGGT
CTGCTCTGTCAGTTACCACTCATCAGTCTGCACTCCAGTTTCTAAAAACTGGGAGTTTTTAGATGAGCCT
CCAGCTTCTTGCAGGATAGGGAGGCCCTGGCAGACATAAACAGGAGAATCCTTGCTCTTCACCCGCTACT
GCCCTCTGCCTAACCCCAGAACAGCAGAAGCTGCTGGCTGTCCCAGCCACACACATCCCTCCCCACTCCA
TCTATTTGTGTGGCCCTTCTTTTTCCCTCAGTCTGTGTCTTGTTTCCTCTCCTTACCTTCTTCCCCCTCA
TCACCTTGGTGATATTCTTTGCATGTTTTTTCTTGGAGCAGTCAGAGCAGGTCATCCCTCCCTCCTGGAA
GAAGGAACCCCCTGAGTACACTCCATGGCACAGTTGACACTCCTTTTCTACCCTCCTTCCCAGATATGTC
TTCACTGTCATTTACACCTTTGAAGCCTTGATAAAGATACTGGCAAGAGGATTTTGTCTAAATGAGTTCA
CGTACCTGAGAGATCCTTGGAACTGGCTGGATTTTAGCGTCATTACCCTGGCGTGAGTATATCTCTATGC
AGATCTGCATGTGTGATAGGAGAGGGGACAAAATGGTGGGTAGGAGACAGGGCTAACATGCAGGTCCCAC
TTGGACAGAGTGTGGAGACTCACACCATGAACGTTTGCTCCAAGAACCACTGCAGGAATATACCAGGAGA
ACTGAAAGAATTCATAGATCCTTTTAAATAAGTGGCACACTGCTGGAAATTCTGCAAGACAGGTGAAAAA
CTGTGAGCTCCCAAAGTGTGAGGGGGGGAAACCTGCCTCCAAACATACATCCCAACTGGGTAATCTGAAA
ATCCAGATCACAGGAGAAGAATTTACCCTTACCTGGAATGTAAATAAATTTAGGAAGCCACACAAAATAT
AAAAGTAGAAACAGCAAAGGGAAGTGCCTTGTAGGCACTCCCAGTCTCCAGCTTGAGACCAGGGAATCCA
TCCCTGACTATGTCTCACAGGCACCCTTGGGGAAGGCAGCCAGTGGAATCAGGGAGGGGTCACAGGATGA
AAGAAGTTTCAACTAAAATTAGTAATAATTTCAACTGGGCACAAATTTTCTTGAGCAGAATCCAGGGGGC
AAATGGGAACTGCAAGCAGACAGTGAGGGATGCGGTCCAAAAGTCATGCTTTCTTTCTCAGTGGGGAAGC
TCACAGCCTGGGGCAAGGTCTGAGTAGGGCACTGCAGGAGCAAGACCAGCCTCACCAACTACATGAGAGC
TAGGTGAGGCCTCTCACTAGCAGCTATCCCCCACTTCCCTGGAGAACTCTATGGCACAGCAGAGGCAGCC
AAAATTCCCTCTGGAACACAACCCCACTGGCCTGAGAACCAACCCCCATCCACCACAGTGGCCACAGCAA
TCCCTACCCAAGAAGGGTCTGAGTCTAGACTCACCTACCCCTGCCCCCACCTGATGGTATTTCCCTACCT
GCCTTGGTAGCTGAACACAAAACCCAGAAACTCTCGGAAGCTTTATGGCCCTGCTCATCACCTAAGAAAC
TAAAATATTTACCCTGGCCAACTTAGGGTGAGCTTAGATCCCCCTTCTACTAATGCACTAGTGCTCTCTT
GAAAGCATCACCTACCGGCTGGAGGTCAAACAGCTCAGGCCATTAAAGCAACTCATGACAGAATAACTTT
GATCCCAGGAAAAAGAAAACAACAACCAGTTGCACTGCCTGCAACATCCTGGCTAACCAGACGTCCTGAG
TCTGTCCATATGACAACTTCACTGCTAACATAACTAGCATTCAAGAAAGCCAGCACACTAAATCTATCTA
CAGCCTAGGACTCTCACAGACTCTACTTCATTCCTCTGCCACCTTCACCAGAACAGATGCTGATATCCAT
GGCTGGGAGACTTCAAGATCAATCACATCACAGGACTCTTGCAGGCATTTCCCAGTACTACCCCAGAGCC
TGGTAGCCCCTCTGTGTGGCTAGGCCCAGAAGAACAATAACGATCACTGCAGTCCAGCTCTCAGGAAGCC
CCATCCCCAGGGGATTGGGGAGAGCACCACATCAAGAGATCACCCATGAAACAAAAGAATCTGCACAACA
GCCATTCAGTTCCAGATCTTTTCTCTGAAATAGTCTACTCAAATGAGAAGGAACCAGAAAAGCAATTCTG
GTAATATGACAAAAGAGGGTTCAATAACACCCCTCAAAAGATCAAACTAGCTCCCCAACAATGGATCCAA
ACTAAGAAGAAATCTCTGAATTGCCAGATAAAGAATTCAGGGCTGGGCACAGTAGCTCACGCCTGTAATC
TCAGCACTTTGGGAGGCCAAGGTGGGCAGATCACCTGAGGTCAGAAGTTCAAGACCAGCCTGAAGCCAAC
ATGGAGAAACCCCGTCTCTACTAAAAAATACAAAATTAGCTAGATGTGGTGGTGCATGCCTGTAATCCCA
GCTACTCAGGAGACTGAGGCAGGAGAATTGCTTGAACCTGGGAGGCAGAGGTTGCGGTGAGCTGAGATCG
CGCCATTGCACTCCAGCCTGGGCAACAAGAGTGAAAATCCATCTCAAAAAAAAAAAAAAAAGAATTCAGA
AGGCTGATTATTAAGTGATTCAAGGAGACACCAGAGAAAGATAAAAACCAACTTAAAGAAATTTTTAAAA
CAACAAGATATGGATGGAAAATTCTCCAGAGAAATAGATATAAAGAAAAAACAATCACAACTTCTGGAAA
TGGAAGACTCACTTAGAGAAATACAAAATGCAGTGGAAAGTTTCAACAATAGACTAGAACAAGTAGAAGA
AAGAACTTCAGAGCTCGAAGACAAAGCTTTTAATAGACCCAAACTAAGACACACAAAAAGAAAGAATTTT
TAAAAAAATGACAAATCGGCCAATAAATTTGGAATCATGTGAAACAACCAAACCTAAGAATAATGGGTGT
TCCCAAGGAAGAATAGAAATCTAAAAGTTTGGAAAACTTATTTGAGGGAATAATTGAGGAAAACTTCCCT
GGCCTTGTTAGAAATATAGATATCCAAATACAAGAAGCTCAAAGAATACCTCTGAAATGTGTCACAAAAA
AGATCATCACCTAGGCAAATAGACATCAGGTTATCTAAAGTCAAGACAAAGAAAAGAATCTTAAGAGCTG
TGAGACAAAAGCCTCAGATAACCTATAAAGGAAAGGTTATCTGATTTACATAAGATTAACAGCAGATTTC
TCAACTGAAACCTTGCAAGCCAGAAGGGATTAGGGTCCCATCTTTAGCCTCCTGAAACAAAATAATTGCC
AGCCAAGAATATTGTATGAAGAGAAACTAAGCTTCATAAGTGAAGACAAGATAAAGTTTTTTTCAGACAA
ACAAATGGTGAGAGAATTTTCCACTACCAAGCCAGCACTACAAGAAATGCTAAAAGGAATTCTAAATCTT
GAAATAAAACCTTAAAATACACCAAAATAGAACCTCCTTAAAGCATAAATATCAAAGGACCTATAAAACA
ATAACACAATAAAAAAAGGAATTAAGGCAATAACTAGCATAATGAATAGAAGAGTACCTCACATCTCAAT
ACTGATGTTGAGTGTAAATGCCCTAAATGATCCACTTAAAATATACAGAAGGGCAGAAAGGAAAAAAAAA
ATCCACCAACCAAGTATCTACTGTCTTCAAGAGACTCACCTAACACATAAGGAGCCACATAAACTTAAGG
TAAAGAGATGGGAAAAGATATTCCATGCAAATGGAAACCAAAAGCAAGCAGGAGTAGCTATTCTTATATC
AGAGAAAACAGACCTTAAAGCAACAACAGTTAAAAAAAGACAAAGAGGGACATATATAATGATAAAATAA
TTGGTCTAATGGAAAAATATCATAATCCTAAATACATATGCACCTAACACTGGAGCTCCCAAATTTATAA
AACAATTACTACTAGACCTAAGAAATGAAATAGACAGCAATATAATAATAGTGGGGGATCAATATTCCAC
TGACAGCACTAGACAGGTCATCAAGACAGAAAGTCAACAAAGAAACAATGAACTTAAACTGTAACCTAGA
ACAAATGGACTTAACAGATATTTAAAAAACATTCTACCAAACAACTGCAGAATATACATTCTTTTCATCA
GCACATGGAATATTCTCTAAGATAGACCACGTGATAGATAACATAACAAATCTCAATAAATTTAAGAAAA
TAGAAATTATGTCAGGTATCCTCTCAGAGCACAGTCGAATAAACCTCGGAGTTAACTCCAAAAGAAACCC
TCAAAACTATACAAATACATGAAAACTAAACAATCTGCTCTTGAATGATCTTTGGGTCAACAATGAAATC
AAGATGGAAGTTTAAAAATTTTTTGAACTGAATGATAATAGTCACACAACTTATCAAAACCTCTGGGATA
CAGCTAAAGCAGTGCTAAGAGGAAAGCTCATAGCATTAAATACCTACATCAAAAAGTATGAAAGAGGACA
AATAGATAATCTAAGGTCACACCTCAAGGAATTAGAGAAACAAGAACAAAACAGACCCAAACCAAGCAGA
AGAAAAGGAATAACAAAGATCAGGGCAGTACTAAATGAAATTCAAATAAAAATAATAATAATACAAAAGA
TAAGTGAAACAAAAAGCGGGTTCTTTAAAAAGATATAGAAAATTGATTGACCATTGGCAAGATTAACCAA
GAAAAGAAGAGAGAAGATCCAAATAAGCTCAATTAGAAATAAAATGGGAGATATTACAACTGATACCACA
GAAATACAAAAAAAAAATCATTCAAGGCTACTATGAACACCTTTATTTGCACAAACTAGAAAATTTAGAG
GAGATGAATAAATTCCTGGAAATATACAACTGTCTTAGATTAAATCAGAAAGTAATAGAAACTCTAAACA
GACCAATAACAAGTAGCGAGATTGAAACAGTAATTCAAAAATTGCCAAGGAAAAAAAGTCCAGGAGCAGA
CTGGATTCACAGCTGAATTCTATCAGACATTCAAAGAAGAATTGGTACCAATTTTATTGAAACTATTCCA
AAAGATGCAGAAAGAGGGAATCCTCCTAAATCATTTTATGAAGCCAGTATCACCCTAATACCAAAACCAG
AAAAGGACTTTTAAAAAAGAACTACAGACAATATCCCTGATGAACATAGATGCAAAAATCCTCAACAAAA
TGCTAGCTAATTGAATCCAACACATATCCAAAAGATAACACACCATGATCAAGGGGGTTTCATACCAGGG
AGGTAGGGATGATTTAACATACACAAGTCAATAAATGCTGCACGTCACATAAACAGAATTAAAAACAAAA
ATCATATGATCATCTCAATAGGTGCAGGAAAAGCATTTGACAAAATCCAGCATCCCATTATGATTAAAAC
CCTCAACCAAATTGGCATAGAAGGGACATACTCCAAGGTAATAAAAGCCATCTATGACAAATCCACAGCC
AACATTATACCAAACAGGGAAAAGTTGAAAGCATTCCCCCTGAGAACTGAAACAAGACAAGGATGCCCAC
TTTCACCATTTCTATCCAACATAGTACTGGAAGTCCTAGCCAGAGCAATCAGACAAGAGAAATAAATAAA
GGGAATCCAAATCAGTAAAGGGGAAGTCAAACTGTTGCTGTTCACCAATGATATGATCGTATGCCTAGGA
AACCCTAAAGACTCATCCAAAAAGCTCCTAGATCTAATAAATGAATTCAGTAAAGTTTCAGGATACAAAA
ACAATGTATACAAATCAGCAGCACTACTGTACACCACCAGAAACCAAGCTGAGGAACAAATCAAGAACTC
AATCCCTTTTACAACAGCTGCAAAAAAAAAACAAAAATAAAAACAAAAAAATCTTAGGAATATACCTAAC
CAAAAAGGTGAAAGATTTCTACAAGGAAAACTACAAAACACTGCTAAAATAAATCACAGATGACACAAAC
AAATGGGAACACATCCCATGCTCATGGATGGATAAAATCAATATTGTGAAAATGACCTTACTGCCAAAAG
CAATATACAGATTCAATACAATTTCCATTGAAGTACCACATCATTCTTCACAGAAATAGAAATAACAACC
CTAAAATTCATAGGGAACCAAAAAGGAATCCACATAGCCAAAGCAAGACTAAGCAAGAATAACAAATCTG
GAGGCATCACCTTAGCTGATTTCAAACTATACTACAAGGCTATAGTATAGCCTGTACCAAAACAGCATGA
TACTGGTATAAAAATAAGCACATAGACCAATGGAAAAGAATAGAGAACTCAGAAATAAAGCCAAATACTT
ACAAGCAACTGATCTTTGAAAAAGCAAACAAAAACGTAAGTGGGGAAAGGACACCCTATTCAACAAATGG
TGCTGGGATAATTAGCAAGCCACATGTGGAAGAAAGAAACTGAATCTTCATCTCTCACCTTATACAAAAA
TCAATTCAAGATGGATAAAAGGCTTAAATCTAAGACCTGAAATTATAAAAAATTCTAGAAGATAACATTG
GAAAAACTCTTCTAGACATTGGCTTAGGCAAAACGTTCCTGACCAAGGACCCAAAGCAAATCCAACAAAA
ACAAAAATAAATAGATGGGATCTAATTAAACTAAAAAGCTGTTGCACAGCAAGAGAAATAATCATCCAAG
TAAACAGACAACCCATAGGATGAGAGAAAAATCTTTGTTATCTATGCTTCCAGCAAAAGACTAATATCCA
GCATCTATAAGAAACACAAATAAATCAGCAAGAAAACAAACAAATAATCCCATCCAAAAGTGGGCAAAGG
ACAAGAATACGCAATTCTCAAAAGAAGATATATAAATGGCCAATGAACAGGAAAAAAAAATGCTCAACAT
CATTAATTATCAGGGAAATGCAAATTAAAACCACAATGAGATACCACCTTACTCCTGCAAGAATGGCCAT
AACCAAAAAATAATAGATGTTGGCATGGATGTAATGAAAAGGAAACAATTTTACACTGCTGGTGGGAATG
TAAACTAGTACAACCACTAAGGAAAACGTTATGGAGATTCCTTAAAGAACTAAAAGTAGAACTACCATTT
GGTCCAGCAATCCCACTACTGGGTATCTACCCAGAGGAAAATAAGCCATTATTAAAAAGACACTTGCATA
TGCATGCTTATAGCAGCACAGTTTACAACTGCAAAAATATGGAACCAGCCTAAATGCCCATCAACCAACA
AGTGCATAAAGAAAATATGGTGTATGCATACCATGGAATACTACTCAGTTGTAAAAAGGAATGAAATAAT
GGCATTGGCAGCAAGCTGGATGGAGTTGGAGACTATTATTCTAAATGAAGTAACTCAGGAATAGAAAGTC
AAGCATCATGTGTTCTTAATTACAAGCAGGAGCTAAGCTGTGAGGATGCAAAGGCATAACAATGACATAA
TGGACTTTGGGGACTTGGGAGGAAAGGTGAGACGAAGGTAAGGGATAAGAGACTACACACTGGGTACAGT
GTACACTGCTCGGGTGATGGTGCACCAAAATCTCAGAAATCAGCGTTAAAGAATTTTTCTGTGCAACCCA
ACACCACCTGTTTCCCAAAACTATTGAAATACAAATAAAGAACTTATTCATGTAACCAAAAACCACCTGT
TCCCTCAAAACTATTGAAATAAAATAAAATAAAAAGATTTGCATGGGTGTCCATGTGGGGCTTATGGGTG
TATCCAGGCTCACACTCTGAAGAAGGTTCTTCATCTCAGTTCAGCTTCCTCATTTGGAATTGTATGGCCT
GGGGTTCTTGCCACAGAGTACCGCTTCGGGGGTTGCTTTCCAAGATTAGTCCCATGCCCATGAGGAGATG
TCAGAGACCTCAAATCTGAGATAACAGTTAGCCTGTCACAGTGGATCAGAAAAGGCCCAGCCTTGTACCC
CCCTGACATGGCGGTCTCCTTCTGAGAAATCAGTGTCTGAGGAGACTGGTTGGTCAAACTCCTGTGTTTG
AAGATCCGTCCACATGCAGTGTTGCCTAGATCACAATGCATCTTCTCAAAGGGTCCACTCTGAAAGATGT
TGCAATAATGAAATCAGTGTCAGTGTCAGTCTATAGATGGCAGTGTCACTAGATTCTAAGTGTCCATTCT
GCATCCTTTCTAGGCTAATGATACCCCAGGTTATCCTTATTTATGCTGCATGCATCCCCAGGACCCTTGC
TTGCAGAGATGCTGACTAACTTCTGGTCCTACAGATATGTTGGCACAGCAATAGATCTCCGTGGGATCTC
AGGCCTGCGGACATTCAGAGTTCTTAGAGCATTAAAAACAGTTTCTGTGATCCCAGGTGAGCATTTATTC
ACAGAGCATATGTTGGTTTTCACAGCCTAACTCCCTTCTCTAATTTTCACAAGAACCCTGTAAGGTTCCA
GGGCAAAGACTATTGTCCCCACTTACAAGACAAAGGAACTGAGGCCTGGAGTATAGGAAACATAAACCCA
AGGCCTCTTCACTAGTAAGAGACAGTGTGACCTGGAAGCCTATTGTCCCAACTCCTAGGCCAGTGCTCTT
CCCACATACCACATGGCCTCACAATCAATACGTAACAGGAGGGGCATGTCTTCACTTTCTGTTCTGGTTG
TGGGATTCAGGCAAGACCCCTCAGTTGAATTTTTATTTATACCTTAAACTAAAGGATTAAACTTGGAAAT
GGGGAGGAAGGGAGAAAGCAGTCCCAGTATCTTCACTGTTTCCGTAGGTCTAAAATCCTGTCTTTAGACA
CCCAGAGGAAAAAAGAATTAGGCAATTCTTGATTACTGAGAATTCAGAAACAAAAGTAGTCCATCAAAGC
TCATTGTGTGGGGCCTCTAAGATCTCCACTGTCACCAGTAGCCCAGCGACTACTCTTCAGCCCCCATGAG
GTTGATGGTGAGGATGGGGAGGCCAGAGACAAGGCAAACTTTCTGAAATGGGACTTGATGCTTCCCCTCT
GTGGCCTGATAGGTTCTCTCCTCTCACCCCAAGAATTCGTTCTCTTATCTGTAAACAGAAACACCCATTC
TAGAGTGGAAGTCTTTTCTACCATTTCTTGTCTGGCTCTGCAGGGAAAGATAGGAAACTCTCTGCCAATT
CCCACTGGACACACCTCTTGAAGAGAGCCCTGGTCCTGCCCCTTCATATCTCTCTCACTTACTCTCAACT
GGGAAGACCCTGAGTCTTATTTGCAAGACCTGGTCTGTCCATACTGTCTCTTCTGCCTCATCTCTCACCT
CTCCTCACATCTCACCATGCTCTGGAATCCACCTACGGTTCTGCAAATACTCCACACCATTGCTCACCTC
CCAGCCTTCTACTGTGCTGCCCCTTCTTCTGGGATATCCTTCCTCCTCTCTTTACTCAACTCACACTCAT
TCCTCAGGGCTCTGCCAAGGTTTTACCTCCTCCCTGGTTTTCCCACCTTCCTCACACTCCACCCACCCCA
TTCCCACCTCTGGAACTTCAGTTAAGGGCTGCTCCCCTGTGTGCCCATCTGCTCAGTAATGCTACAGACT
TTCCCCTCAATTGTCAGACAACGGCACTCGACATGCTGCTGCCTTAGCCATCACTCAGCTCCCAGTCCAG
GGACTGACACATGTAGTGCTCAGTGGATTATTTGCTGACCAAAATGACTAACCAAAAATAAATCCTAGAT
TGCTTCCCTGGTCAGAGCTCTTTCTGCTACCTGCGAAGCTGCTCTCCCAAAGAAGTTGATTTTACTTACC
AGAGCAACAAATCATCCCTCCTTAAACAAGCAGGTGTATCCTTCTTTGCTGCCAGCTGTGCTTGGCACAA
GTTCTATTTATGCCCCAGTGACTGTCTCAGTGTTTCTTATAGTCTCTTTCTATTGCTAAACTCACCTCTC
TGTTTCTCTGTGTCTCTTTCTTTTTCTTTCTCTTGGTCTATTTTTGTTTCACTCTATCTCTCTTGCATCT
CTCTCTCTCTCTCTTTCTCTCTCTCTCTCTCTCTCTCTCTCTCACACACACACACACACACACACTCACA
CACAGTGAGGTTCATGTGTCCCAAGTCTCAAGCTGCCTCCCAGATGCATGCAGAAAGACAGATGCCAAAT
TACTGGGGTGAAGTTAGTAGCTTCATGCCTAGCACAGTGCCTGGAAAAAGTAGGTACTCAGCATAAATTA
TTTAAATTGAATTGCTAGTGTCAGGAATTTTATTAGCCTCAAGATGATAAAAAATGCATTGCCTGGGTCT
CAGTTGCATCGTCTTTATAATGAGAATAGCCCCTGACTCTCTGCCTGCCTCTCAGAGTAAATGCAAGCTT
CGGATGAGAAAGGTCTTGGAACTGCCCTGGAGTGTGTACATACTTCACTCCTGTATGTAGAGATGAAGGA
GGATGGCTAAGATGAAGAAGTGTGCTCCGTGTGTGCTACCTCATCCATGTGGTCATACCACTGTCATCTT
CCCGCAGGCCTGAAGGTCATTGTGGGGGCCCTGATTCACTCAGTGAAGAAACTGGCTGATGTGACCATCC
TCACCATCTTCTGCCTAAGTGTTTTTGCCTTGGTGGGGCTGCAACTCTTCAAGGGCAACCTCAAAAATAA
ATGTGTCAAGAATGACATGGCTGTCAATGAGACAACCAACTACTCATCTCACAGAAAACGTGAGTGGAGT
CTCTTCCATAGGGACCAAGGTCTGGTTGGACCCTTGGTATCATATAGGGACAGGGGATATGGAGCCCCTG
TGTGTGTGTATAGACATATATTTTGACTCCCCTTTCCTGCCAAAATGGTTCTCTTGAGTTGTGTTTTACT
TTTGGGTATTCTATTTCCTGTTGCTCAGCATCCCTCTTGCAAGGAAATGAGCCATAAATCTAGAGGCAAG
TCATTCTACCTCTGCTGGGGGAGATTGAGAGCCGAAGTCACTGCTTAGCTCTTCTCTTTTCAGATGTGAC
TTTACAACTTCCATGTGGCCCAGCATGCCCACTGAGTACATGTAGCCAAAGGAGCTCACTTTGGAAGACT
TGGGAATATTGCATTCACCACACAAGGCTTGGGTTGGAGGGTTCAGAACCATCTGTGAGGCTTTCTTTTC
TTCCTTTCTTCAGCAGATATCTACATAAATAAGCGAGGCACTTCTGACCCCTTACTGTGTGGCAATGGAT
CTGACTCAGGGTGAGTTCCCAACTTATGACAAAGCACGAGTGCCCAACAATTCCTTCTCCATCTTGTCCT
TGGCATATTATGGGCACAGATGGGGTTGCAGGTCCTGGGAAAGATGTATAAATAAATAAATCATGGCCTT
TGCCAACCCAGAGCTCATGGGGAGGGAATGTATGAGTTAGCTTTCGCTGTGTAACAAATGACCCCAAAAT
TTTGTGAATTAAAACAACAATGTATTTAATTCATGATTCTGTGCATCAGCATTTTGGACTGGGATCAGAT
GGGCATTGGGTGACATTGACTAGGCTCATTCATGCATATGCAGACAGCATATTAACTTGGAGATGGCTGG
TCTAGGACGTCCTGTCTCTGCTCATGTGGCCTCTCACCCCCAAACAGGTTAGCCTGGGCCTGTTCTCATG
GCAGTGTTCCCAGAAAAACAGTGGAAGTTTGCAAGGCTTCTTCAGGCCTAGGCTTAGAACCAACACAATG
TACCTTTTGTACATTCTGATACATTCTTGACTAAAGCAAGTTGCAGTCCAGTAGAGATCCAAGAGACAGA
GAAATAGACTTCATCACTCAATGGAAAGAGCTGAAAAGTCACATTGGAAAGGGTGTGGATACATAGAGAC
AAGAATTATAGCCATTTTAATAAATAATCTACCCCAGGAAGGAACCAATACTTATTTGTGTCAAATGCTC
TACACTTAATCATCACAGCTAACTCCATTCTATAGCCACATTACCCTCACAAGTAGAGGAAAAAACACAT
GTATCAGACTTTGCAATTTCGAATTCTTACAGAGGCCAGATTCATAACAACAAATGAGTGGAGTGGGCCA
GGAGGGAACCTAGGAGCTGAGAGTTCATGCCTCCTCTAAAGGGGACAGCCCCACTGCTCGGTCCAGCTTT
TGGTTGCCATGTGGAAATGTGGGTTCACTGTTTCCAGCTCTTCCAACTTTTAAAATAGAGATAGGAAATT
TAGATTTTTATATAAAATCTCCCAACCGTAAACATTGGCTACCTATTCCAAAAAATGTCTACATATTGTT
GGGCCAAACAAAATATTTCTGTGGGCCAAATCCAGCCTATAAGCTGCTAGTTTGCAATTTCTCATTCATA
GACATAAAGATGCTACCCTGTGGAAAACAAGTTCTTTACTAGAGGCTTCATAGGCTGGGCAAACACAGTG
AGGTGGCGGTGTGAGGCAAGAGAGAAAGTAGGGAAGGCTTCCTGGAGGTGGTGTTTGAGGTGAGCTTTCA
AGGGCAGGTAGGATGTCTCCAGGCTGGGTGAATACGAGATAGTTTCAGCGAAGACCATTCCAGGTGGCAG
AACAGAATGTGAGACAAAGCTAAGGGATGAGGACCCCTCAGCTCTGCCTGCTGCTTTGGCCACTGGAGGA
AGGCATGAGCAGAAAGACTTTAGGCAAAAGATCAAAAAGGCCCATCTCACCCACCTCCATGGCTGAGAGC
TTGGAAAAGCAGAGCTCACAACACCCCCAGTGCCTGAGAGGCTGACGGCTCCATGTTGCCCAGGTCGACC
TGTGTGGAGCCTGCATGAGGCTCCACCTTCATCAGCTCACTAGGAAAACTATCTCAGTTGGAGCTGAGGA
CTGGAAACAAGCAGGAGCATTTGGTCAACATCAGGTTCCTTGGACTGTTCTTTCACAAATGCACTTGGCA
TTTGGTTCTTTGGCTCATGAGAGGGTTGTTCTGACATCAGTTTTCATTTGCCTTTGACTTAAATGTAAAC
CAAGAGCCCCTCAGCATCCTTCTGTCTAGATAATGGCATTTCTCAAGGCAATGGCTCCAGAGCAGCTCAA
GTCCTTTTTGTGGTAAAGTTGTTGGGTCCATCTACACACCTCTACAGCTCAGCTCCCTGAGAGGCCAGGC
CTGCTATTGGGCAGGTGCCAGCCAGGCAGTGGGGGAGTGGACCAGGCTGGGATGGCAGAGAGAGGCCAGC
TCAGGATAACGGACAGAGTGAACCCTGCCGGGGCATGCCGTGAAAGCTAAACCTGGGTCCTCTGTGGGGC
TGCAGAGAAGCCAGAGTCCTGAGGAAAGGCCCACATTGGCCAATGGCACTGTGGGAGGAAGGTCAGGGGT
CTCAAGGCAGAAAATGGATACCAGAAAATAGTTGATAATTTTCAGTGTTCTTCCACATATATTATTCCAG
TTTATCCTTTCAGCAGCTTCCAAGGTTAAAAACAATAGGAATTTTCCTTTGCAGTTTATATACAATATTT
ACTATGCAGAAGTGCAAGACACTATACAGTGATCTCATTTAATCCTCAGAGCAGTGAACTGTTTATATGC
AGTGGCTATGGTGGATTTCTACAGAAGACATAGAAACAAGGCTGCCCTCAGAGTTAATTAAGAAACCTAC
AGGAAATGGGTTGCCTAGCCCCTTGGAAGAGCAAACCAAGAGCCTATCTTTAAATGTGGGTCTACAAAGC
CAGGGAACTTGCAATCTTCAATCGAAGTGGAGATAGACTTGGAACTGATTAGAATTAAGTTGTCAGAGCT
CTAATCCATGGTATGGGTTGTTCACATGGATGTCTGGGATAAGGAGAGCCCCAGAGGTTGGAGTTCATGG
AGAAATCCTGGGAGTAAAAGAATTCCATGTATTCACAAGGAATGCAGGTAAATAAAACAGGAGAAAAAGA
AAAAAATTTAAAAAAATAATTCTGGACTTCAAACGAGGCATTGCTGTTTCATTGATCAGGCCATCTCTGC
TAGGGTGGATGAGAGCACTTTACGCTCCCTGAGCTGACAGATGCTTCTTGACATCGACTCATCCTCCTAT
TATGAGAGAGAGGAAGGAGTTCAGCATTCCCACCTCAATACTCACCAGGCAGTCATTTTTAATCTGGCCT
TCCTTCATAGGCCATCTTCAGGAGGAGGCTAGGCTGACTCAAGTATTGGGCTACTTGACAGTTATATTTC
TCCTTTTAATAAATATATTGACACCTGCGTAGTTTTAGGCAACTGACTTCTTTCTGACCCCCAGATTCCT
TATCTGTAAAGAAAGAACATTGAGTGATTAAATTATAGTGTTTGTAAAGCACTTAAAGGGAGAGAGAAGA
ACACAGAAAGTGCTCAGTAAGTGGAAAGTGTTATTTCCACTGTTACATATAATTAATCAGTGAACTTGGC
TAATGTGAGACCTCCCTTGATAGACAACTCTGCAGATGATGCCAACAGGCTCAGGTTTTAAGTCTGCGTT
ATTGCTACTGACTCTCATTTCCCTGGTATGAACTGGCAAAGAGCTTATAGTATCGTGTCTGGGCAGTCAG
CTGCCTTCCCCAGGCCACTCCCCTGTGTATGAAATTCTGACGTGAAAGTATTAGCGTCCCCGTTTTATGG
ATAACAGAAACCAATACCTAAACAGACCAAGTGTCCAAGATCATCCTGCTAATAAGAGGAAGACTAGGTC
TTGAACTCTGATCTCTGACATAAAACTCTGTGGTTGTCTCGCAGCCCTGGGCTACCTTGTCTGCAATAAG
CAGGGGATGACCTTCTCTGTTCTTGCTGCAGCCACTGCCCTGATGGTTATATCTGCCTTAAAACTTCTGA
CAACCCGGATTTTAACTACACCAGCTTTGATTCCTTTGCTTGGGCTTTCCTCTCACTGTTCCGCCTCATG
ACACAGGATTCCTGGGAACGCCTCTACCAGCAGGTACTGAGCTGCATTCCCCCACGCAGACTTGGTTGGA
GGCTGGTGGGTCAGCTGCATGGCTGTGCTTTTCCTGGAGGAGGCTGACTTAAATTCAGGGGTCCATGTGC
TATGGGTACAGGTTTCTCTTCTTTCCCTTGAACCTGCAGACCCTGAGGACTTCTGGGAAAATCTATATGA
TCTTTTTTGTGCTCGTAATCTTCCTGGGATCTTTCTACCTGGTCAACTTGATCTTGGCTGTAGTCACCAT
GGCGTATGAGGAGCAGAACCAGGCAACCACTGATGAAATTGAAGCAAAGGAGAAGAAGTTCCAGGAGGCC
CTCGAGATGCTCCGGAAGGAGCAGGAGGTTTGTGAGGAGTGGAAGCTTTGTGAAGAAGGTTGCAGACTGT
CCATTCTTGGATACTGTTCTGGACTAGCCACTTCAATTAGGGCACCGTTTAGGGAGCTATAGGAGGAATC
ATCTTGCCTCAGGGACCATATTAGATCAGGTTTTCTCAAAGAAGAAGCTAAGTGAGGATTCTTGTGCAAG
TGACCTATTGAGGGAGTGCTCTCAGGAGAAAGCGAATGAAGCAAAACAGAATGGGGCAGAGGGTGGAACT
AAACAAGGAGTGTTCTCAGCTAGAGACCAGCTTCAGCTTGATCCCATGGGGAACTCTGGGGCATGACCTG
TACCACAGTTAGTCCTACCTTGAGGCAAGGGGGCCAGCTTTTTTTTTTTACCCTCCCCATACCTATCCCA
GAAGCAGGCAGCCAAAAGAGACTTTTTTTGTAGAATGCTGCAAGTATGAGCCATCAGCTGCTGCCATGCA
AGCAGCTCGGGATAGCTGTGCTGACCTGGTGAAAGGGATCTGGGTGGGGCACCAACAGAGTACCCCATGA
ATGAGTGGGGGTGACCCGAGGTGTCTGTTCCTGCCATGGCCTAATCCCACCACCACCCTGGTCCTAACCC
TTCAGCTTCTCTGTTTCAGATCTCTCACCCCTGGCACCCCATGTCTGGGGGACACATTCATGCTAAGTCC
AAGCAAATACTATTGGCTACACCTGCTGTTCTCTCTTCGCAGGTGCTAGCAGCACTAGGGATTGACACAA
CCTCTCTCCACTCCCACAATGGATCACCTTTAACCTCCAAAAATGCCAGTGAGAGAAGGCATAGAATAAA
GCCAAGAGTGTCAGAGGGCTCCACAGAAGACAACAAATCACCCCGCTCTGATCCTTACAACCAGCGCAGG
ATGGTAAGAGGCTCAGGTTTGTGCTCTAAAGATTACCCACCATTGCAGCTGCCGTAGACAATGAGTTGGA
GTGGGAAGAGGTGTGTGGCAGAGAAAGAGCTGGAGGCATAGAGCCTAAAAGGATCCAGACACCCCTCCAA
AATGGTGTCTTAAACTAGGAAAGTGTAATTATAGATGGGGAAAAGGGGTGAATTATAAGGACACCTTAGG
GAAGTGGATCAACAAGATTGGGATTGTGGACTGTGGAAAAAGGAAAGGGGAGTCCAATTTGACTCCCAGG
TTTCAGGATTGGGCAATCAAATGGGTGGTGATTCCAAGATGAGGACACTGGAGGAAGAACCCCGGTTTGT
AGGAAAGGTGGGAAGCCCATACTGAACATGCCAATCTTAAGGTGTCTGCGGACATCAAAGGTCAGCTTCC
TTCTGAGGCTCTGGTCTGCAGTTCTAGAGTGCAAGGTTCCCTGTCCGTGGTGCTGAGATCCTTCATGCAC
CTTTTTTTTTTCTGATGCGTCGAGGTGGTTTTTATTTTTTTACTTAGTTTTTTTTAATTGACACTTCACA
ATTTTATATAATTATTGGGTATAATTTCTGACCTCCTTCCTAAGGTCCTGCAATTCAAGCTGTCACCTGT
TGTTATCAGAAAAGTTTCCAGCACCCATCTCTGTGACACAATCCAGCCTGGGAATGGCTTCAGCACCTCA
CCTCAGACCCTGTAGCAATTCTTTTCCCCCCATAATAACTTTCTGCCTCCCACTCTAAAATTTGCACTGC
AACTTTCCTCGCCACGCTTCCTCCCACCACCTTCCTGCCCCTTGAAAGTCCTTTTACCCCAAATATGCCC
TTTCTATAGGTCTGACAGATAATATCTGCTGTGTGGGAGGGTGGCTCCACACTAACAGTGTGGAAAAGGG
GATCAACATGGCTATTTTTTTATGCTTTATACCTCTCACTTCCCTCTCAATCTCTTTTCCTGTGCTAAGC
CGAAGGATATTTTACTTTTTTGCAATACCATCTCTCCAACCCTCAGATTTATGTGTGCATATACAAACAC
AGACACACTTGCATGCACACTGACCTCATGACACATCCACACTTCAGCATTTATCTGCATCTGTGCCCTC
CTCTTTCTTGCTTTGCCCCTTTTGAGCAGCACCTGGCATACATGGGGCACAGTTCAGACAATCACAGAAC
AGGATGAATCACAGCTTCCATGGAAATGTCAATGACACTGAATTGGGCTTTAATTTCCCCCATTCTGTCA
GCCCCACACAGATTAGCACTTGGATGTCCAATAATTGGTATGTGTGCTTGTCTCTCCAGTGATGCTTACT
TTCAAAGGGAAAAAAGAGGAGCTGTAACTTGTTCATTCAACAATACTAGGTGCTTGGTATAAAGAGATGA
CTAGAACATGTTCTTTTCCCTAAGGATTTCCATTTGGGTGGAGGAGGCAGATATAAACACAGCTATGTGT
TAAATAGTGTATTAGAGACACATACATGGTGTTTTGAGAATCAGGGAAGAAACATCCACTCTTATGGGGC
TGGATAGGTATCAGAAAAGGCTTCCTGGAGGAAGGCATAATTGGGATCAACAATAAATAGCATGTAGGGG
TTCTTCTAGTAATTGAAGACTTGAGAGAGTTTGGGGAGGAGTATTTTCCTGGCAGAAAGAACAACATGTA
TGTGCAAAGACAGGGAGGCACCATTTAAGTACTTCTTTAATGCTAATTGATTTATTGGAGCCCACTCAGG
AAGGCATCCTCTCCCTCTTTTCCCAGGTACATTCATGTGGTAAATAGACCTAATGCAGTTTTTATCTAAA
GCCATTTTATAACCAATAGAAATTAAATAGAGGAATATATAGAAGAGACCCAGGAGGGTTTTTGTGGAAA
ATCACAGACCAGTTGGTCTCGTTAGAGGATGAATTTTAAAGCAATTCTAATGCTTATACTCAGAAGACAT
TGCCCACCATTTGTCAAAACCTTAATTCTAAAATAACTCGTGGCCAAGGAGACACTGCTGTCAAATATGC
AACCAAAATTTTCTTCTCAGTTTCTTCATTGGTTTGCTTGGTGCTCAAGTGCAAATAATGTAGTATTCCC
TTCCTTAATATGGATGTGTCAATTTTTCTCATTTGCTTCAAAAATTGGAAGGATTTTTGCATTTTATGAG
TTCTATAGTCCTTGGGCATGTATTACAGGCAGAGAGTACTCTATTTATAATACTGAGCTTTTAATAATGG
TTGCTAAACAGGATCAGGCTTCAAATTATCAAGAGAGGAAACTTCAGTTCTGTGAGATAAGGGGATTAGT
AAGGAGAACCCCAATTGGCAAGATTCTCCACTTTAGTAGCTTAGTAATAAAAAGGAGTTAAGGAAGGTGA
GTGGAATAAATCGGTATGATCAGAGTTTGGTATGAAGTCAATGTTTAGATTTTGTACATACTATAGACCA
CCTGAGGACACAAATGATGGTTTATTATGGTCAAGTTTCACTATGGTCATGGGAACTACCTAACCCAGCA
CACTAATTCTCTGAGGGCTCTGGGTTTATGAATTAGCTCTTTTCTCTGGCAACTTTAAGCCACACAGTTC
CCTCAATGGAAGATTTTGATGGGAAATGTTTATTGATTTGGAGGAATGACATTATTCTTCCAGGAAATTA
GGCTGCAACCCTTCCTTCAGTCTCTCCATTGTTGACTGTTCAAGCCTCGAGGGATCCTAGAAACTCATTG
CACTGTACAGCTCTTGTCTCCTCCTTTGCAACACAAACTCTCACATTCATACAGATGGGATCTAATGAAT
TGTACTACCTAATTTTCAGACAATGAAGTGCACTTATTCATTCAACAACCACTTACCAAGTATGTTCCAG
TACTGGTGTGCATAGTAGCCTCCTTAGGTCACAACTTTGCATGTGGAAAAGTGAACTTAGGACAGCTTTG
TCTAGCCCTGTGGCCAAAGGTTGGCAATCAACTGAACTATCCAGGTGACAAGGTTTCCTCTGACATGTTC
CTGACCATGATAACCTATTTGCTGAGCATCAGCACGCTGTAATCTCTCCTCTCTCCTGGTCTTTCCCGGA
GTCTATCTAGAGTCTCCTTTTTTCTTTTTCCCCACCATGCATACCTAGAATACCCCTTAGATTTGTGCCT
TTACTTAGTGACTGAAAGGTCATAAAGACAGGGGCAGGGAAATGGGAGTAGGTAGAGGGAAGTGTTGGGC
TCTAGATTTCCCAGAGGACCAGTTTCCAGCCTTCTTGCTCCTTTGGGGCAATGCCCAGTCTTTTCTAGGC
CTCGCCTCTGGAAAACGCCGGGCTAGTCATGGCAGTGTGTTCCATTTCCGGTCCCCTGGCCGAGATATCT
CACTCCCTGAGGGAGTCACAGATGATGGAGTCTTTCCTGGAGACCACGAAAGCCATCGGGGCTCTCTGCT
GCTGGGTGGGGGTGCTGGCCAGCAAGGCCCCCTCCCTAGAAGCCCTCTTCCTCAACCCAGCAACCCTGAC
TCCAGGCATGGAGAAGATGAACACCAACCGCCGCCCACTAGTGAGCTTGCCCCTGGAGCTGTCGATGTCT
CGGTGAGTTTGTGAATGCTTCAGACTCCCTCAGGCTGTCTTCCACCTAGAAGCCTTGAGCCAAGAAGCCT
GAGTATCACCAATGGAAGGGATCTTTAGCCATCATCCTGTCCAATGCCTCCCCATTCTACTCCTTTTTTA
CAGACATAGAAGATGAGACCCAGAGAGAGAGAGAGATTGAGTTGGCCTGAGGTCAAACAGGTTTCCTGGT
TTATTCTGCAGTAGTGATGCAAATTATATTCCATTTGACCCCATATTTCTTATCAGTGCTTCTCTGTCCC
CACTCAACATCACTGCCTCAGTGATAGGCCTGCTTTCTTGTTGGACAGGTATGAGAAAAACTGAAGGACC
CTTCTCTTCCTGCTTATGGCTCTATGCAAACTCTTCTAGGCTGGGCAAATGGCACCCAGAGAAATTCAGG
AAGTTTCTTAGGACAGATCTGAATAGAAGCACAGTATTCCTTGCTTTACTCACAGAAGAAATAATTTGCC
AAAATGAGCAAATTAGGCAACAGTGACTAACTGGCCAGCACTCTTTAATAGAATAATCGCAGAGTGTAAT
GGCAAGTCAGGGATTCCTCATTGGATTGGGAGCTGTGAGTTAATGTGAGCTCTCCCCTCCAGTTTCAGCA
ATGATGTTTCTAGTGTTTTTCATTTGCCCTCTCTGGCTAGCCAGGGATACAGCGCAATTGCAGACAGAAT
TGTAAAGAGACCAATAAATGGTTCCACTGATACAGATAAGTCTTAGAGTTGCCTTAGCTCAGTCTCTGAC
TCAATGTCATGTGATTCAGGGTCCCAGCAAGAAACAGATGGACCACTCCGATGGGCTAATAGAGAGAGTT
TAGTAAAGTATTTACAGAGGTGTGGGCTGGGCTTAGGGAATGAATAAGGGACAGTGAGGCACCTAGAAAC
TAGTAACAGGGGGAAGCTGTTACATGCCTAGGCTGAAGGGATGAAGGAAAGGACCAGAATTAGACTACAG
CAGCAGCTGCCCTCATGTGTCAGAGAGGCTGCCTGGCAGAACTTGTGATCTTAGTAGAGGATCACAGCTC
TGCTACTCAGCCATAGCACAGGAGAGAAGAAGCCAGGGGAAAATACCTGACTTGTCTCTCCTTCTATCCT
TGAATGTCCTGCCTATGTCTCCCATTAACCAAACCTGGCCAGAACCAGAGGGTAAGGATAACACATTTTG
TACAGGTCTCCCTCCCCAGGAACAGAGACAGTGGAGAAGGTAGAGAGTAGGTCTCCAGGGCAAATGGAAT
CACCAAAACAGAGCCTTAAAGTCAAAGGCATCCTGCAGGGCTCGCTGGGAGTGCATTAGCACTCTAGAAA
ATGCCTCACTGAAGGCATTTATGCCTCTCTTGTGATGGGCCCCCACCCTCCTTTCTCTGTTCCTGAGATT
ATCATGTGTCTAGATATTCTGACCTTGATTCATTCTAGTTAAATTACCAGGCTAGTGAAAGATTGACTCT
TTAGGCCCTTCTGTTTAGTATGGACCTATATTTTAGTCCCAACTGTGCCAGTTACCACCTGAGTCACATT
TAGCAAGTCCTTTAAATTATCCAAGCCTCCTTATCTCCCCTTGTAAAAAGGTTTCTAAATAGCTGGAGGG
TTAAATGAAATAACAAAGTGCCTACATTTTTATACATGTCTGACAGATAACAATAAATAGTGACTACTAG
TATCATTTGCTTAGTCCTCATGGTTGGAAGCTTTATAAACTTTCTTTATGTTTATTAACTGTTTATACTT
ACAGTATTCAGCAATAACATGCTGTACAGATTTGTAGCCTAGGAGCAATAGGCCACACTATTTAGCCTAG
ATGTATAGAAGGCTATAGCATTTGTTTGTGTAAATACAATGTAGGATATTCACAGAATGATGAAATCACC
TAATGATGTATTTGTCAGAACATATCTCTATCTGTAAGTGACATATGACTATATATGAGATTGGCCAATA
AGAGGCTGAATGATCCAACTTTGCCATGAAGATGTCAGGTTAAAGGAGCGTCCTGACTCTGTTTGTGTCT
CAACAGGCATTCGATGCAGGACAAAAGAAGACTTTCTTGTCAGCAGAATACTTAGATGAACCTTTCCGGG
CCCAAAGGGCAATGAGTGTTGTCAGTATCATAACCTCCGTCCTTGAGGGTAAGTGCTGCTCACTGCATTG
TTCATCCACTGTGTCATGATGAATTCCCATGTGAGCAGCAGAAGCAATGTCTCTAACTCGTGTCAAAGCG
TCCATCTCTGTGTACTGGAATTCCTAATACTCCTCATTCTTATGTCTTAGGCCCCACCATCTTTTATCAA
TGTGGTTTTTTAGATGCATAATTTTTCAAAAACAAAATAATTTGATGAATAAAATATCCTAGCTCATCTC
TTGTTCAAATTAGCATCTGCTTTGCATGCATCACAAAACTAGATTTTTAAAAGTGATCTGGCAGGCATCA
GAGAATTTCAGGCCAGCTCTCCGGTGTACAAGAAATTTAACCATCCTTAATTTTCCAACATGGCAAAATT
ATCCTTTTCATGGTTTCATAATTATATTTTTCATGGTTGTAACTTACACAGTTGTAATTTAATTTCTTGT
TCAAGCCCACATTATGCTGAATATTGGTAGACAATTTAAGTCCATTAATCAGTCAATTTAACTAAGTCCT
TGTTAATTTAGAATTCTACCAATCAAGTGGGAATCCACTCAGTCTCCCCAGCAATGTATCTAAGTTCCAA
GCCAAAGGGTAGAGGAAGAGAGTGGCAATGGTCATGGCCACCTATCCACACACTTTGCCATAGAGCGTGA
TTGCATCCCTCTAACCTTGAGCAGGAGGTCTGTTCTAATCCAGCCTTTTTTTGCCTTAGGGTATGGAGAA
TGCAACCTTCATCATTTCCAGTTATCTTACTCCCCTAGACAAATTAAACTGACCTAGGCTGGAGGAGTTG
TAAGGGAGCTTGAGTGCTGGTGTGCCCCTGAGAAGGAGTCCTGTGTGTTTTCCTCCCAGCCCTAGGCTGA
ATCCTAATTTGAGGGGAAGAAGTGGTGAGCAATAGAAACCAAAAAAATGTTTGAGGGATTTCAAAACAGA
GCCAATGTGTGCCCTACTTTCTAGGTAAAGCCAGAGGTGGCAAGCCTTAGAGAGAGATTCACATGCACAG
AAACTGTAGAGTAGAACTAGCCAGTAATTTACCAGATATGCAGAGATGTATCTGTTTTGCTCACAGCTGG
ATCTCCATTACCTAGAAGAGCAGTGCATTAACAAAGGTGATATCTGGGGGGTGATGCTACTTTATAGTGG
TGTAAACTGATAAAAAGGAAGAAAATAAGGAATAGTCAGGTATCTTGGGGTAGGATAGACTTCCGCTCAA
GGAAGATACTGTGAGAGTAGAGTAAGAAAGGCAGGAGAAACAAAGACGTTTTTAAGAAAATCTGGGGAAA
GGCAACCTTAGCTTAGGTCAACCTTAGGTTAGAGAAGGCCTGAAGCAGCCTCAGAGAGAGCCCTGTGGCT
CCAGAATGGCAGAGAGGAGCTGGGTGATGACTTAGCCCCCAGTGAAGGCTATGGAAAAAGATGAGTGAGA
CCCATAGAGGGAAACTAGAGAGAGAAGAGCCATCTTCATGGACTCTCCCACCAGGGACCAGATGGGAGCT
ACAACTCAGGCAAAATACGCTTAGCAATGACAAAGGACCAGCTCAACTCTCCTCCAGGTTTGAGCCCTAC
AAAACTCAGACACAGACTCAGATATTGAAATAGTACCCAACATTGACTTAACAAGAGACAGTTCCCATGG
TGAGGACTTGAAGCATAAATTGAATTCAACTAAGTTACATTTCTTGCATACCTGAGTTTGTGCTTTGAGA
AATGTGTACTCATTTCTCAACAACAACGTGTACAGACTCTCTTCTATTATTTTTTCTCATTGATAATTAT
TTAATATCACTGTTCATTGTAATAGAGTCTGTACTTCTAACATTTTTCATGGCATAGAATTTGACCAAGC
TGCTATTCCCACAATTTCTTTCTTGGCCAGATAGGTTTCTTGGTTTTTGTTTGTTTGTTTTGGTTTTTGT
TTTGCTATTAGTCATCAAACAGCAAACATTGAATACAAACAGTTTGTTTTTTTCATTTATTCTCGAAAAC
TGTTAAGCTTCCTCCTTCTCTCTTCTTTCATTTATTCTCTTATCAACAAGCATTTGTTGAGCCCCTGTTG
TGTGTGTGGCATTGCTTCAGGTGCTGGGATTCAGTGGTGAACTGATATAAGCTTGCGCTCATGGAATCTA
CATCCTAGCAGGGAAGGGGACATGAAAGATGAATAGATATTCAGATATAAGAAGTAGGAGAAAAAATAAA
GTAGGAAAGGAGATGGAAAATTCTCTCCAAGAAGAACAGAAATTTGAATTAAATAAAGGAATCAGCTATG
TAAGAAGCTGAGGAGAAAGTTCGCAGACACAAATAGCAAGTACAATGCATTCTGCAAGAATGAGATAGCA
ACAAGCAAAATGCATGTCCCAAACACATCCCAGTGTAGGTAAGACAAAAGAAGGTCATGGGATGAATGAG
AGGCAACAGAGGGTAGTAGTTAGAACACTGCTCTGGAACCCGGCTGCCTGGGTTTGCATCCTAGCTCTTC
CACTTATGGTACACAGTGTGACCTTGGAGCTTCAGTAACTTTTCTGTGCCTCAGATTTTTAGCCTGTAAA
GTAGTGAGAATATACTTATCTTGTAGAGTTTTGTGAGGATTAAATGAGTCAGGTCAAGCAAAATGCTTAG
AACCACGACTGGCATGTAGTAAATACTCATCACATTTGGCTATTATGATTTACTAGGTTGTAAAATCTAT
GAGGGCGGAGATGTGTCTGTTTTGCTCACAGCCTGACCTCCATTACCCAGAAGACAAGTGCATGCACAAA
GGTGATATTTGGGTGGTTGGCGGCGGGAGTGATGCTAATTTATAACAGTGTGAACTGATAAAAAGGAAGA
AAATAAGTAATAGGCAAATATGTTGGGGTAGGGTAGACTTCCCCTCAAGGAAGATGCTGTGACAGTAGAG
TAAGAAAGGCCAGAGAAACAATGATGTTTTAAAGAAAATCTGAGGAGAGGAAACCTTAGGTTAAATAAAA
GAGGTCAAAGTTTTCTGAGCATAGGGCACATCATCTTCTCTGGTTGGATGTTATATTCATTACACACATG
AGACACCTGAAGGATGCTTCATGCTGCCAGAGTCGTAATAGGATTTATAAAGGCCACCTCTTTAAGAGGG
ATTATGCTTAAGAAAGTTTGGCCCAAACCAACAGGAGTTTGACAATTCACTGAACTCAAAGCATGTGAAC
CTTCTATGCTTGGCATGCAAGCTGAGATTTCACTGTTGTCCAAGCCAGAATTCCAGAAGCTGGGTCACAG
CAGTGAGCTTACTGGAGTGGTGAGTGCCTTAAAGAGGAAGTGCAGGATACTAAGTGAAATATGGTAAGAT
TTGCCTGGAGCCAGGGAATTATCCCAGGCTGAAAGGACTGCAAAGCAGAAGAGAGAATAACATTTGAAGG
AATGAAAAAGCCTAATGTAGCTGTAACCTAGAGAACCAGGAGAACTGAAAATAAGGCTAGAGGGATGAGT
TGGGGCCAGATCCCAAGAAACTCCTAGACTGCATTAAGGATATGGGACTTCATGCTAAGAGGAGTGAGGT
GCCATTAAAGGATTGGAGGCAGGGAAGTTACATTTGCATTTTATAAAAGTTACTCTGGATACTTGTAGAT
ATAAGAGGGGAAGTATGTTGTCAGGGAAACCAGTTAGGAGGAAGCCAAGTACAAGATGGCAGTGCTTGAA
CTCTGGGGTAGTGACAGTGGGGCTGATGGGAAATGGAAGGATATGAGAGCTATCTAAGGGGCATAAGATT
CAGGACTTGATGATGCATATGGTTCTGGTATAAGTGCCTTAAGATATGCTGATACAATAACTCATAAAGG
GAGCATTTTAGGGGAAGAAGGGGGCTTTAATGGGGAAAGATGAAAGAAGATTATGAGTTGAGTCTGGGAT
ATATTGGGTTTCAGGTGCCCTTGGAGCATCTAGGTAGGGGTGTCCAGTAGGAAGTTGACTTTAGGATATA
GGGGTCTAGAACTTAGATGACAAAGATGGCATATATATATATATATATATATATATATATATATATACAC
ATACACACACATATATATATGTATATATATATATTTGAAAGCAGTTTGAATGTAAGTGGTAATTTTGAAA
AGATGCAACTGGATGAGTTAACACAGAAGGGTTGAAGTAAGAAGAGAAGAAGGCCTCAGACACAGTTCTA
ACAAACTAAATGTTTAATAGTTAAGCAGAAGAGATGGACCCAGCAGAGGAGACTGAAAAGGAAGGTACAG
AAATAGGAAAAGATCTAGAAGAATACAATGCAGAGCCAAGAGTAAAAATTTCTTCAGAAAGAATAGAAAG
GCCAACAGAGGCAATGCTGTGGAAAGGTCAGGTAAAATGAAACATGAAAAGGTCCAGGGGCTTTAGTCAC
ATGGATGTCTTTGGTGATCTAAGCAAAAGAGGATTAATGGAGTAAAAGGGTAGGCAGAGGTCATATTGAA
GGGGCTTAAAGAGCGGGTGAGAGGTGAAGAAATGGAGATATGGTGTACAGACAATTTTTTCAAGAAGTTT
GCCTGTGAGGAAAAGGAGATAGTAAGATGGCACTAGGGAGTAGGGAGAATTGAAAGAAGACTTTTTGGTT
TTTGTGTTTGGGGGAGTTTTTGTTTGTTTGTTTGTTTTGAGCATATTTGCATGCTAATGGGAAGGATCTG
GGAGAATAAGAGAGACTGAAGATTAAGGGAAGAAAGGGTATATTCAACAGACTCCTGCAGTGGCAGGAGA
GAATGCACAGTGGAGACACTGGTGTTAACAGGAGCAGGGGGACCTCTTTAAGCGTAACAGGATGAAGGGA
GGAAAGTTCTCTCTGTTATCAATTTTATATAGGAGATCCTTTTGAGGTGGGGCCATGGTCATGTTTTAAG
CTGGCAAGTAGATACTTTTAGTCTCTGTGTCTCTGCAATCAATCAAGTTCAACAATGTCAAGGCTGTTTA
CAAAGATGCCTCTCTTTTCCCATCATGCCTCACAGGTGGCTGCTTCCTGCCAAAATGGCACATCTAAAGT
GTCCTCATTCATGACTACTTCCTGTGCTTCTTTTTAGTGCCAAAATAGGTCCAGGGAAGTTCTTGCTTTT
GTTCCAAACAGGGATTGTTGATTTTGGTCCCAGGATGTGCCAACTTCTTTAGAGAACTATGCTTTGCCAG
TTTGGACATCTGTAGTTTCTAAAATCCAGTCAGTGATTAAAACAGCATGACAGTGAAAATAAATAGTCTG
ATCTGTGGAAGAGAAATGGACATGTGTATATGAAAACACATGAACATAAATGGTAATAACATGACAAAAA
TGACATTTCAAATCTTGGGAAAATGAAAAATTATTCAATAAGTAATGTTGATATAAATAATCAGAGACAG
GAAAAAATAAACCACATCAATATCTTACTCTTTACCCAAAATAAATTTCAAATGCACCAAAGTTTTAAGT
GAAAAAAGTGAAACTACAAAAGAAAGAAAAAAACATGGAGAAGGTATTTTCCAAGCCTTGTAGTGAGAAA
AGCTTCATATGTAAGGCATGTATGCATATGTGGCTAACCTGCATAATGTGCACATTTACCCTAAAACTTA
AAGTATAATTTAAAAAAAGAAAAAAATTTAAAATAAAAAAATGAAGAAAATTGAAAAAAAAAGAAAACAA
ATAAAAATACTACTGATAAATCTTGACTACATACACATTTTTAAATTATCAGGAAAAAACACTGTTAGCA
AAATTTGGGGGCACACAGTTATTTCTCTAAACTGATTTTTCAAGTGCATATAGTAAAAAAAACCCTAAAC
ACTGCAAGTGTGTATGCAATGACAACTATGTCTCCTTCCCACTATAGACCCTTCCTGCTCCCCAGACTCC
TGGTGCACCCCCCTCCCACTCCAGTTTATCTTATATTCTTCAAGCAATGTTCTATGCATATATAAGCACA
TGCATGCACATACACACACTACACATACATATTTTTCTACATCACCACAAATAAATCTGCCTCATTCTTT
TTCACAATGTATTATGCTTTATCAGTTGTTGTTTTCCATTATTATTATAAACAATGGACATCATCTTTCT
TTGCCCACATAGGCTGATATCTGTTCAAGATGCATGTCTTGCCGAGGAATTTATAGATAAACCTAAAGCT
AAGAAATTGCCCTCCATCGAGGTGAACACAGTTACACATCCACATGGAGATTTAACGGAGGCAGGTAGAG
AGATAAGCCTAGTGTTCAGGAAGGGATCAGAAATTGGACACATAATTTGGTGGGAATTGGCATAGAGATG
GTTTTTTAGGGCTATGAGATTCAAAAACACAGAGAAGCTAAGGTGAGAATGCTTGAGTTATTCAACATTT
ACAAGCAGGAAAGGAAAAGGGAGACAAAAACAGAAAGTGAAAAGAAGTGGAGAAATATGGGAGAAAAAGC
AAGAGATGATGCAGTCCCCAGAGCCAAATGAACACTGGTTTCAAAGAGAACAATATAAATTCTGTCAAAT
GTTATAAGGGTAAGATGAGAACTGGAAATCAATTATTTGAATTTGGAAGCAGTTAAAGTCAGATTCCAGG
GGCTGCAGAGAAAAAAAGAGGTAAGGAGCTGTAGAAGGTTAAGTGTAGAAAACTCTTGCAAGGAATTTTA
CTGTCAAAGAAGCAGTAATGAAAGAGAGCATGGGGTCCAGAGAGACTTTTTCAAAGCCGCGAGATAATTA
AAGCATGTTTACCTGCTCATGGCAATGGTCTGGAAGGGAGACAGGTCTGGATGATGCAGGCCTTGAATAG
GTGGGAGAGGAAAGAGCCCGGCACAAGTGGGAGGGGTAGAGCCACCCACGCACTGTGTGGGGCACGAAGA
CAGAATACCAGGTGGACTTGCAGATATGCCAGAGATTTAGTGGTAGGAAGAAGGGGAAGTTCTTTTGTGA
ACTAGTCTATATTCTGAGTGAAATAAAAGGGAGCTCATCAACAGAGAGTAAGTAGAAAGAAGAGCCGGAG
GTTAGACGGGGGAAGAGGAAGTGAGAAACAGTCATCTGGAAGAATGAAGGAATGGAATATAATATAGAAA
CACAGCCAGATTTCTGGGCAGGGCAAGGCTGGGGCTCACAGATCTCTAGGAGGGTTGGTAAGCATAGTTG
TGTATTTTTTCCAGCCAGGTCCAGCTGGTTGGGTGCAAGTACAGAGTAGTCTACAAATTGGGTCTATTCA
CAGTTCAGGTTTTGCCAGGCAAGTACAATGAAGGAGGAGAGGGGCAAAGGAATTGAGGGTGCCTACAAGG
GAGTAGTTAGAGAGATGGATGTGAAATCTAAGCTGGGCAAATTGAGAAGTAAGGACATGATATAGGTGAT
GGGCAGTAAAAATATGTAATGTCAGCAGTTTAAAGGACTGGATGGGGCAGATATTAATTGGAGTTGCAGG
ACTAAAGGAGTTCAAAATATAGGAAATGAATACCAGAGACAGAGAGAGGGCTGAAGTCAAAATGTTGGAG
GTGGTACTTATTATTAACAACAAGGTCTAGAGGATGACCGCAGAATTGGGGTCCAAGGTGACACATGGCT
GACAGCTGTCATTGACCACACTATAATGCAGAACTCGAGGAGTCTGAACAGAAGTGCCCACCCTGCTTGA
CCAGCTTGTCTCAGAAGTATCTGATCTGGGATTGCTGCCCCATGTGGGTGAAGCTCAAGACAATTCTCTT
TGGGCTTGTGACGGATCCCTTTGCAGAGCTCACCATCACCTTGTGCATCGTGGTGAACACCATCTTCATG
GCCATGGAGCACCATGGCATGAGCCCTACCTTCGAAGCCATGCTCCAGATAGGCAACATCGTGAGTGCTC
TGGCTGGTACTGCCCTCACTGGCGTGGTCGGGGGGGGGTGGGGATGATGGCAGGGTGCAGTTCGGGTGGG
GTGGGGCATGCCCTTATTGGTTAGTGTGTTGAGCCAGTTAGCTGAGAGCAGAGCAGCAGTGCAGGAGACA
TGTGGACACTGGGTTCAGGAGGTCAAGTGTGGGAGGACCCAGGCTGAGCTGGTGGGAATGGCTTTGGGAT
GAACAAGCACAGGAAAGTGAACCAGGAGAGCCTGGAGCCAGAGGCAGCTAGTTGCCCTTTACAAAACATA
GGACAACCACTTGAATTTTCTGGATCTCAGTTGCCTCTACCTTGAAATGAGGATAAATGTCACTTTTTTA
TCTAAAGACAATCTTTGGACTTGTCTGTTTCCTTCCAATGTAATTACTTTTATGACCACAATTAACAGTA
GCCACGATTTTCAATGTTGTCACTTAAGGAGAAAAAGAGTTGGGGAGGGAAGGGGAAGACAGTGGAAGGG
AGGAAACATGGAAACAAGGTTGCTGTTGTAACAAAAGCATCAGGAAGTCGGAACTTTTCTTTGGCATCTA
GTTTAGACATCATGCTGCTTTAAATTTTCCTCCATCACTTGTTAGTATTATTTACTCTTAACAAGATACA
ATCCATACTGGATTTCACTATATACTGGATAAAGTATCTGACTCAGAGGGACAACAACTGCTCCAACCTT
GCCCCACCCACAGGAATAGTAATAATACTCAGATGCTTCTCATGGATTATCTCATTTTATTTTCACAACA
ACCCTACATGGTAGGCACTCTTGTTATTCCCTTTCCCAGATGGAAAAATGGAGATGCAAAAGGTTAAATA
ATCTTCCCAAGATCATAGAGTTGAGTTTAGTGGCCACATCAGAATTTGAACCCAGACAGACCGGCTGCCA
AGCCCGCTTTTCTTAACCCCTGAATTAAGCCACCTCACAAGACTTGCGTGTGTGTGTGTGTGTGTGTGTG
TGTGTGTGTGTTCAAACACACGCACACATGATAGGCAGAAGCTAGAAAGGGTCAGCATCCTGCCTTCATG
TCCGCAGCAGGGTCCACCAAGCTGGAGGTGACCTACTGGATTCACCAGAACTGTCTGGCTTTGGGGTCCA
GCTTCTTATAGGCTGCAGACTTAGGTGGAGGGGGAAGGCCCAGAGGAAATGTGACTACTTGTCCCTAGAA
ACCACTCCCTCCGCCATAATCCAGGGCTGTGAAAGGGGCAGCAGTAGGTTCTGGAACATGGTGGAATCAG
CATGCCTCATGTTTTGGCAGCACCTAGGCTGAGGTCACATCCTAAAGTTGATATGAAAAGGAAAAATGGA
AAGAGAAGAGAATGCTTATTCCCAAGAAGTCAGCCTAAGACCACAGAGGGCCTTATGGAATAGACATTTG
AGCCCACACCCTCATTCTACAGTTAGAAAAGTGGAAGCAAGAGAGAGAAAAGGCCTTTCCCACAGTGTCA
GAGGAAAGTGCCACTTTTTTAAAGCAAGAAAGTCATGTGCTTGGTGCCTGCAATGAGTCAGGTGCTTTAT
GAATGTTATTTCAGTGTCACAACAACATTATGAGGTTAACATCACCCTCACATGACAAAAGAGAGCACTA
AGACTCAGAGAGGTTAAATATCCTGCCCAAAATTGTATAGCTGGTGAGGGTGGATCCAGGGTGGACCCAG
GTCTCTGGAAGACACAAGACCTCAACCCTACCCCCCACCACACCACACTGACTTGCCTACAAGGGACACT
CCATAGATACTCTTTGAATCAGTCTTGTGGCATGAGAAAGTCACTTAAACTCTCAGCCTGTTTTCTTCTC
CATAAGAAGGATGCATTGTCTGTTTCTCAGGCTGGTGAGGGGCTCAGATAAAAAGATATGAAGGAAAAGC
ACCTGACCCTGGTCGTTGCTTTCCTCCTTCTCTGTCTCCCTTCCCTGCTGGTTGTTGTGTTACCCGAGCC
TGGTTCTGACCTCTCTCAGTGTCAGAATTCTGTCCTATTTAAGACAATTAGAGACACACAGAGCCTAAAA
GAATCTTAGAGGCAATGAATCATTGTTCTTACTAGTCACTGTTACTTATGAATGAATTACTGTCTGCCAG
GCCCTTTGTTAAGAACTGCACATGCATTGGCTCATTTTATGCCCATTATACAGATGAGGAAATTGAGGTA
CAGGAAGGATGCATTATTTGTCCAAGCTTACATAAGCTAGGAAATGTCTGGGCTGGGATTTTAATCCAGG
TCTGTGATGTCCCACAACCCACACGTTTTAACTACTGAACCAGACTGACTCTTCACTGACAAAATGATAT
TATAATAATGATCTTAAAGACTATCCGCTTATTCCACTCCCACCACCATTCACGTGGAAAAACTGCAGCA
TGCACAGCCCCTGAGAATGGTGCAGAAATGTCTCCATTCTGTCACGTTGACTGTTACCCTGGCCAATCCT
CAGGAGCCCACAGATAACATAGCAACTGAACAAAACAAAAGGATTTTCTATTTTGTAAATAAGACATTTT
CTTGCTGCCATTTTTATCATTGCCTCCGACCCCACAGATCCCACTGTGATGAAAGCTTGTTTCCACTCCT
AGGTCTTTACCATATTTTTTACTGCTGAAATGGTCTTCAAAATCATTGCCTTCGACCCATACTATTATTT
CCAGAAGAAGTGGAATATCTTTGACTGCATCATCGTCACTGTGAGTCTGCTAGAGCTGGGCGTGGCCAAG
AAGGGAAGCCTGTCTGTGCTGCGGAGCTTCCGCTTGGTAATGTTTTTCTCTTGATGGCTTGGGGAAACTC
CCACCCAGATTCAGGGAAGACACTGTGCTCTGGGGACTGCACCAGCCAGCTCCCCCTTCCTCTCCTGTCC
TTGTGGGAGGGAGCAGGGTCTGACTCCTGGGCGACTGGAGACTTGGGTCTGTGGCCCCAGCTTTTCGCTG
ACTCAGAGTGGGGACATGCCTTCTCCTTTCTGGGCCTTGGCTCCCTTATCTGCACCAAGAGGGGACTTCC
AAGCACTGTCCTCGTCTCTTATCAAATAACAATAACAGCAATAATCACAGCAGCTAATACTTACTGAGCA
CTTACAATGTGTCAGGCATAGCACTGGACACTTGAAAAAACGCACCTCATTTTATGCTCACAACAACCCT
GAGAGATAGTCGTTTTTAAGATCCTCAGGGGTGGGTGAGGAAAAAGAATGGTTCCATGATGACCCGAAAG
TCTCCCATCTGCCAAGCAAGCGAATAAGCCACAGACCTGGATCTCAAATCCAGGTTGTCTGACTCCAGAG
CCCCAGCTCCTTCCAGCCTCAAGGAAGTCATTCTCTGATTCCCTTGTGCCCGAAATCACAGCCCCAATGT
CTCAAGCACCTTCAACCTGCCTATTAGTCTCACTCACATGCAATGATGGCTCTTCTCTAAACTCAGCTCC
AGGACGCCCTCTACAGGAGAAGTGCCACAGGCCACCCACTTTTATCAGAGCCACAGGTTTGTCAGCCAAA
CCTGTCAGATTCCCAGGAGTGTCGGGTCACTAGGTCTCCAGGCCATCTGGAGGAAGCTTGGCATCTCACT
GACTCTTGGGAATAGGAAAGAGGCCTGAAGAAATGTCACGGCTTGTTAGCGCAGGCCGCCTGCTCTCAGG
AATCTCTCAGTATTAACTGGGAGCCCTGCATCTAGACTTGAAGAGAGGTGACAAAACAAAGGGGAGTATC
CAACAATCTTCCCAAGGTCCTGACAAGTGTTGGCATCCAGAGGTTGAAGGGGGTGCATGGTTCAGGAGGC
AACGAGACTCTAGGACAGATCTGCTTGGGACCCTATCACAGGGGCCAGTGAGACAGTGCTGAGCCCCTGA
ACTTGGCGTACCTGGATTAGGGGTGGCAAACTCAAATGCCCATGTGCAGTGACATAGAGCCAGCCATGGA
AGAGATGTGGGCACTGGATATTTGCCTCAGGTAGACAGGCAAAGATGACTCCATTTCACCTAGGCAGACC
CAAAGCAGTGCACAACTCCTGGGAGGGATGTTTACATAGAAATCAATGTGACCATCTCAGGCCAGGTGCA
GTGGCTCACACCTGTAATCCTAGTACTTTGGGAGGCCGAGGCAGGCAGATCGCTTGAGCCCAGGAGTTCA
AGGGCAACATGGTGAAACCCCGTCTCTACAAAAAATGAAAAAATTAGCCAGGCATGGTGGTGCACATCTG
TTATCCTAGCTACTCAGAAGACTGAGGTGGGAGGATCACCTGAGACCAGGGAGGTGGAGGCTGCAGTGCA
CCATGATCGTGCCACTGCACTCCAGCCTGGGAGACAGAGTAAGAACCTGTCTCAAAAAAGAAAGGAGACA
CAGAGAGAGAGGAATAAAGATAGAAAAAGCGAGAGAAAGACAGATGAAAGAAAGAGAGAGAGCAAGAGGG
AGAGAGAAAGAAAAAGAGAAAGAAAGAAAGAAGAAAGAGAAAGAAAGAAAGAAAGAAAGAAAGAAAGAAA
GAGGAAGGGAGGGAGGGAGGGAGAGAGAGAGGGAGGGAGGGAGGGAGAGAGAGAGGGAGGGAGGGAGGGA
AGGACGAGCTATGGCCTGTCCATTATTGTCAGAGTTACTAATTTTCCAAAAAAGTCAGAAATCCAGATTT
TTGTGCAAATGTCTTTAATATTGGCTACTAATCCAGTTTTTAAAAACACTCTAAAGACCAAATAAAGCAT
GTCCTGAGACACAGCTGTGCCATGAAACGCTGGTTGATGGCATCTGCCTAAGCTGATGGTACTGTCACCA
TCCCAGGGAGGAGAAGGGTAGAGTAGGCCTGGTAGGACTCTTTATCCATAACTTGAAAATCTTACAAGGT
CAGAATTAAATCACATTTTTAGAAGGACAGATTTCTCTAGGTATCTAGAAATTGATGTCAAGATGTAGGC
CCCTAAGCTAATGGTCAATATAAGGTACCTATGCACAATCGTCAAGAGAGCAGGAGATATCACACCCCCA
TACCCTGGCAGGCCCACAGTTAGTTTTCTAATAAAATACGAGGCTGGGCGCGGTGGCTCACGCCTGTAAT
CCCAGCACTTTGGGAGGCCGAGGCAGGCGGATCACAAGGTCAGGAGATCGAGACCATCTTGGCTAACACG
GTGAAACCCCGTCTCTACTAAAAATACAAAAAATTAGCCGGGCACGGTGGCGGGCGCCTGTAGTCCCAGC
TACTCGGGAGGCTGAGGCAGGAGAATGGCGTGAACCTGGGAGGCGGAGCTTACAGTGAGCCGAGATTGCG
CCACTGCAATCCGGCCTGGGCTAAACAGCGGGACTCCGTCTCAAAAAAAAAAAAAAAAAAAAAAAAACGA
ACATTTCTGCTACACTTGGGGAAGCAGAAGCTTAGTGTTTTAAGGTCTATGTGCTACTTTTGTTCTGATC
CCACAGTCTAAAGTCCCAAACAACCAAAAATAGGCAAGTTTACTTCACAGTCTGCCCCTACAGGATTCTG
CAGGGGAATGCTACCCTGTCCCCACCTCGCTGATTTAACATGCTGATCAGTGCTCAATTCCCTAACCACA
CCCACCAACTACACACTTTCTTGGGATCTATTCCACTTCCCTCATGACCTAGCTTAGATTTCACAGCCCA
CCAATATTATTATGCCCTTGAAAACAAACTCAATCACCTTTTCCCTCTGCCTCTTTGCGTTTTTGTGTTT
GCCAAAACCTCACCGGAGAGTCTAAGTCAAATATTTCTCTCCACCTTGTCTGCACCTGAGCTGTGAGAAA
TGCTGGAGAAAAATCACCGCCAGCTGGTCAGTTTCCTGGTTTTAATTAATAGACTTTATTTTCAGGACAG
TTTTCAGTTTTCAGAAAACTGGAGCAGATTGAACAGAAAGCTTTCATATACCATCAATCCCACAACACAC
ACACACACACACACACACACACACACACACACACACACAGAGTTTCCCTATTACCAACATCTTATGTTAG
TACCTACATTTGTTACAAATAACTTCACTGGTTTCCCATTGCTCTGAGAATAAAATCCAGACTCCTCATG
ATGGTGTACAAAGTTCTGCATAATCAGGACCTGCCAGTTCACCAGTCTCCGTCTTGCCCACTTTGCTCCA
GCCAAAAAGGCCTACTCTCTCTTCTTCACACATAACAGAGAACTTCTTGGCTTTGGGCCTTTGAAGTTTT
TTTCGCTGTGCCTGGAATACTTTTCCTTTAGTCTTCACTTAGCTCACTTATTCTCAAACTCTGTGTTCAA
TGCACTTGTCACCTCCTTAGAGGGGACCTCCCCAACCACACTATCTAAAGTTGGATTCCTTCATCAAATT
AACCTGGCATTTACAATTGTCCTGCTATCCCATGAGGCAAAAACCAGAGTTTCTTGTCTACCGTTTTATC
CCAAGGGCCTAGCACAAGGCCCAGCACATACATAGGAACTCAATAAACACCTGTTGATTGATTGAAGGAA
GGAAAGAAAGAGAGGAAGGTGGAGGGAAAAAGGAAGAAATAAAAAGTCTGTCTTCCTTCTCTCCCAAAAT
AGCATTGCTTCTCCTCCCGTCACTTAAAGCCTTGCTCCATTTGCTCTGTTAGTCCATTGGTCTATCCTGG
CACCAATATCAGTCTTAATTATGGCAGCTTTAAATATATCTGTGTCTGTAAATCAAGTAATCCACCTAAT
TTACTTCAAGAATATCTTGGCCTGCACACTTTCATATTCACTTTAGAATTATCTTGCCAAGTTACATACC
CACATACACACAAAACTGCTATTGTTTTTCCCATTAGGGACTGCTGTAATTGATAGGTCAATTTGGGAAG
ACTTATAATCTTTACATAATTCAGTCTTCTGATCTATGAAGATAGTATACGCTGTATACCAGTCTTTCAA
TAAAGTTTAAAAATGTTTCCATGAGGTTTTTATAATTTTCTCCATAGGATCTTGCATATCTTCTTAAATG
TTCTTTTCTGGCATTTTTGGCTAGCATTTTGATATTTAACATTTTTGTATCTAGAAATGTCTCTTATTAA
TTCTAATAATTTATCTATACAATGTTTTAAATTTTCTAACATAATCACATCATGTGAAAATAACAATGAG
TTTAATTTCTTCTTTTCTTTCTTTTTTTTTTTTTTTTTTTTGAGACAGAGTCTCGCTCTGTCACCCAGGC
TAGAGTGCAGTGGCGTGATCTCGGCTCATTGCCAGCTCCACCTCCCGGGTTCACGCCATTCTCCTGCCTC
AGCCTCCTGAGTAGCTGGGACTACAGGCACCCACCACCATGCCTGGCTAATTCTTTGTATTTTTAGTAGA
GACGGGGTTTCACCGTGTTAGCCAGGATGGTCTCGATCTCCTGACCTCATGATCTGCCTGCCTTGGCCTC
CTAAAGTGCTGGGATTACAGGCATGAGCCACCCACGCCCAGCCTAATTTCTTCTTTTCTAATCCTTATGC
CATTAGGTTCTTTTTACTAGATCACTGTGCTAGTTAGGACATCCAGTACACTGTTGAAAAGAAGTGTTAA
TAACAGGTATCTTTATCAATTTTCTGATCCCAAAAAGAATCCTTTCAATATTTCACCACCTCTAACAATT
GTGGCAGGCTTTTTTTAAAGATAAGAAATCTTTATCAGTTCAGGAAGTTCTCTTTTATCAAGAATATTTA
TCATGAATGGATGTTTAATTTTATCAGACAATTATTCTACATGTATTGAGATAATCATGTATTTTCTCCT
TCAATCTGTTAAAGTGGTGAATTATATTAATTGATTTTCTAATGTTAAATCAACTTTACATTCCCAGCTC
AAACCTAGCTTGATCGTGATGCATTTTTTAATACATTGGAAGATTTAGTTTGCTAAAAATTTGCTTAAGA
TTTTTTTATCCATATCAATGAGTAATATTTGATCTATAATTTTCCTGACTTTCATTGTCCTTGCCAGGTT
TTGGTATCAAGGTTCCAAGATTATGCCATTTTTTTTTTAAAGCCAAGGGGGCCAGGTGGGGTGGCTCACG
CCTGTAATCCCAGAATTTTGGGAGGCCAAGGTGGGAGGATTGCTTGAGGACAGGAGTTGGAGACCAGCCT
GGCCAACAGGAGAAAACCCCATCTCTACTAAAAATATATAAATTAGCTGGGTGTGGTGGCACACACCTGT
AATCCCAGCTACTCAGGAGGGTGAGGCATGAGAATTGCTTGAATCCCAGAGACAGAGGTTGCAGTGAGCT
GAGATCATGCCACTGCACTCCAGCCTGGGCAACAGAGCGAGACTCCGTCTCATTGAAAACAATAAAAAGA
AGGCCAGGGGCAGTGGTTAATGCCTGTAATCCCAGCACTGTTGGAGGCGCAGGCAGGCGGATCACCTGAG
GTCGGGAGTTCGAGACCAGCCTGACCAACATGGAGAAGACCCATCTCTACCAAAAATACAAAATTAGCCA
GGTGTGATGGTGCATACTTTTAATCCCAGCTACTTGGGAGGCTGAGGGAGGAGAATTGCTTGAACCCGGG
AGGCGGAGGTTGCGGTGAGCTGAGATTGCACCATTGCACTCCAGCCTGGGCAACAAGGGCAAAACTCCAT
CTCAAAAATAAATAAATAAATAAATAAATTTTAAATTTAAAAAAGAAAAGGAAAGAAATTAGCTGGGCAT
GATGGCACGTGTCTGTAGTCTCAGCTATTTGGGAGGCTGAGGTGGGAGGATGACTTGAGCCCAGGAGGTC
GTGGCTGCAGAAATTACAGCATAAATTAACTCTCCATTAGTTAACTTAATCAAAGCCAAAAGTTAGCTAA
AATCTTTAACCTTCCTCCAAGACAACTTTTCATTCCAGATATCCTTTCCCAATTTATTGTAATGATAAGT
ATTTTAATTTTATCTTGTTTATAAAAATTTTTATTAATCAATGAAAAATAAAGCTACCTATTATACAGAC
AATCTCTAAAACTGAGTAACTGACCCCAATAAAGGTTTATTTCTCACTCGTATCATAGCCTAATGCAAGC
CAGACAGCTCTAGGTGGGACTCCAAGAAGTGACTTTGCCATGCCTGAAGGAGGCCAATATAACTTCAAAG
TTTGCCTCCTGCTTGTGGGAAAATGGAAAGCCAAGGTTTGTTTTAAGGTTTAAACAGTGAAAGGCTTTTT
CAGTCAGGGATTTTGATTTATTTCACAATACAATTACTTCAAAAACTTAGATGGCTTCAGACCTCTCATT
GCCACACAAAGTGCTTTCTTTGACAAAGTTCTCAATTGTTCTTTATTTCTTTGCTTTCCTTTCCCCATGC
CTTTCTCTTGCACAAGAAACAGTGGCTACACCATTACTACAAGCTTTGCTAAGAGTTGTTTCATTTAATT
GGAAAATTTAACTAGTCTTCAACATTTGAAGACTTAATCCCATTGCTCACTGCCTGGAATCTAGAAGCAG
TACAGTTCCCTAAATCGTCAAGGCTCTAAACTTCTGGATTATATTTTCTTTTATTCTTATAAACCAGCTA
AATCTTTCCTGAGCTAATCTCTTTCTTGTACTGTCTTGACCAACATAGCCAATTACAACTGACCCACAAT
ACTAAGGTTTAATAAACCCTTCCCCTAGGGTTGCTGGTTCATGAGGCTCGTGCTCTGCCTCAAAATTTTT
GCCAGCAATATTTTTGTCACTGAATAACATAGATTGACATTCCAAGCCTTCTCCTATCAGCTTCCTTGTT
GCCCACCACATGATCATTAAGCCAGTGCCACATAGTTGATGCTTTTGAAAAGGCAATACCTTTTTTCAAT
GAAGCAATTTCTCCATTACTCAAGATAAGTACAACTAGCTGCAGTGATACCCTCTAAATCTGTGAGTTAA
CATAATAGAGATGTATGTCTCACAGCACATTCAGTGCTTGTTGGGAAGCTCTCCTGGGAGTGTCTACTTC
AAGTAGCGCCTCAAGGATCTAGGATTATCCCATATCGTGATGTCACCATCTTCAGTTAGTGACCTTTATG
GTGAACATGAAAGGGAAAGAAAAAGTTCAGGGGGTTTAAGGCTATGCCTAAAAGTCATGTATATTACTTC
TGTCTCCATCTCCATTCCACCAGACAGAACCCACTAACATGACCCCAACTATTTGCAAGCAAAACTGGAA
AACAGTCTTCCGTTGTGTCTGGGAGGAGAAAAGTGAATGGAGACTATATGACCAGTCTCTTCCACATCTC
ACAAGACATTGTTGTTGTGTCCACTCAATGTTTATTTAGATTCACTCTCATGCTTAGCACTTTCTTGCTC
TTTATACCTTTGTTATAGAGCCAGTGGGGGCTCACTGCCCAATGCTCTAGAAGCCAATACTATGACTTTG
GAAGCCAAGACGAGGACACCAGGCTTTTGAGAAAAGAAAAGCTTAATACTGCAAGCTGGCTGGCAAGAAG
ACAGGAGGTCAAGCTCAGATCTGTCTCCCTGTGCTGTCTTCAAGGCAGTATTTTTATTAGAAAATGTTTA
GTGGGTGGATACCGGGATTTGCAGGTGATTGGTGGAAGGAAAGGGGAGGTTTGGAGAGTCCATGGGCCTG
CACAGTTATCTCTTCATGCTTACTCATGGGTTGCATATGCAAATCTGGGGGGAGTTAGTATGCAATATGA
AGTGGAAATTCAGTCCATGACGTCAGCAAGCTCATTCTGCACAAACTCCAGTCGGCCATCCTGGTTCCAA
TTGATTTCAGCCTGTTTTTTCATCATCACACAAGGGGAGGGAGTTTCAGTGTCTCAGCAAGTTGTTTCTT
TACCTATCTGCTATCCTAAAAACTCAAGAATTTTTGTAAGTTACTGATTTCTTACTCTTTGGAACACAGT
TTGAGTTTCAGATTTTCAGTGAGTTATTTCTTATCTGGTATCCTGTAAGCCTAAAAATTTAGTTATTGAT
TTCTTCAACCCTTTGGAGCACAGTTTCACATTCTTGTATATTACACATTACATATTACTTCTGGGATCAC
TTTCTATCTACCTGAAGTACCTCCTTTAGAATTTCTTTTAGTAAGTGCCTTCTGGCCATAACCTCTTTGT
TTATCTGAAATTTGCTCTTCAGTTTGCCCGTGTCTTTGAGAGACATTTCTTATGAATGTAAAATTCTAGT
TGATACTTTATCTTCCTTCAGTGAAGATAGTATTCCAACAATTAGCCTCCATTGTTGCTAATGAGCAGTC
AGCTTTCAATTTAACTGTTATTCCTTCCTTTCTGGATGCTTTTTAAATCTTCTATCCATCTTTGGTGTTC
TGCAGTTTCACTGTAATATGTCTAGGTGTGGACTTCTTTCCATTTATAATGCTTAAGATATACTGACTTC
TTGAATCTTTAGTTTTGTAATTTTCTTCAATTCTGGAAAGTTCCCAGACATGATGTGGGCAGTCATAGCC
TCCACGCCATTCTATTTTTTCTCCTTCTGGACTTTGGTTAGATAGATGTTAAACTCTCTCATTCTATCCT
CTATGTCTCTTACACTATTTTGTTATTTTTCATCTTATGTCTCTCTAGGTTACATTCTGAATAATTTATT
CTGATATATCCTCAGTTCACTAATTCTCTCCCAAACTGTGTCAAATCTGATGCAAACCTTCTTCATAATA
TTTTTAATTTTAAGTATTATATTTTTCATTTCTAAGAGTGTATTTTTCACAACTTGTTATTGTTATCTAT
AGTTTTTTTATTCTCTGCACATAGTTTCAAGTTATTATTTTATTTCTTTAACATATTATATCTAATTATA
CTGTTATCTCTGACTAATAATTCTATCATCTGAAGAAATGGAATGACTATTTCCACATCTGTGGTTTCTT
CAAGATCTCATTCACTCTTGATGACTCATTCCTTCTGTTTTTGTTTGTTTTTTTACTGCAACTCTTAAAC
AATTTTTGAGGGAATAGTTTGAAACCTGAGATAAAGGCAGGTTCCTCCAGAGGTATTTGCATTTAATTCT
GCAGAGCACTTTGTAGAATTGCTCTAAATTAAATTCTCAACTTAAAATACCTAGGTAGTGTGAATTCCAG
ATAAAATCCTATATGAGCACCACTTTGTTGTTTCAAATGCTCAGCGTCAATTTTCCTCCTGTAGCTGGTA
CCTAACACCAGCCATTCAATTTTCCTTGCATTCTCCTGGGATTGTTTGTGGTTCATCCTTACATGAAGGG
TATAGCTCTTTGGGATCTAGTCTTAAAGCAGGGAGAAATTCCTGCTAGGCTTACAACCTTCGGCAGGCCA
TGGACTACGTTTCCTGCTCCCCATACTAGTTACTAATGCTCAGTTTTTATTTGTGTTTATCAGCTGCCTC
TGTGACCAGTCCACAGAGTTGTTGTTTTAAGCTCTGCTTCCCATATGAGGAGAGTAACTTCCAGATTGGG
TGTAAGGTGAACGTGGACCCGCTCTCCCCCATCACATAAAGAGGTGCATACAGCACTTACTGTATAACAG
GATTAAACCTCCTCCCGTGGGATTTACTCACGGATATGGGTGATAAAATTGTAGCTACACTGTTGTGTTC
AGCATTGCATCCTAGCACCTAAGCCACAGCCTGGCTCTTAGTAGGTGCTCCATAAACACACCTTGACTGA
CAGACTGACAGTTGAGCTCAGATTTTTTTTCCTGACAAATTAGAGAACCATACAAATATATTTTAAAACA
CATACATTTTTATGCTTGATGATTTTGCTGTCTTGTGGGACTTAGCATATTTGAGTTTACAGTGAAGACT
TTTGAGACTTCATCTTTTCTCAGTAACTAAACCATTGGTTCCATGAGAGCAAGAGCCCACACACCTCTCT
TCATACACCTCATAGGCTAGCACACTGCCATGCACCTGCACGGCTGGAGTTCTGGGGGGTAGGTTCGTTG
GTCAATTAAATCAACTTATGAATGCTGAGTCCATGAAATCATGGACATTTGCAGTTTCAAAGCTGGTGGG
AGCAACAGATGTTGTCCACTGCCCCAAGATGAGAAATGCCCTAAGAGATGTTTTGCCAATACCTATCGAA
TGCAGTGGGTCATCCCCTACCCGAAGGAGGCTGGTCTCTCTTCAGTCATAAATAAGCTTGTTCTCACTTA
TTTGTGAGCCCCATCACACCCACCCCACCTCCACAGAAGAGGCCCATATTCAGGAGGGATCAGGCCTGTG
GCTATAGAACAGAGTACAGATCGCAAGGCCACCACCCCAGTAGAAGACCCTGACTCAGCCACAGTCAATG
AAGCACATTGATACTTATGAACCATAGAAGGCAGATCATATTTTTGAGTAATTTATAGCATAGGAAGAAA
GGTCTCCAAAAGCAGTGTCCAGAAAACGCCCTGGTTTAATGGGATGAGAGGCCTTGTGTTTTTCCCAGAT
CTTTGGTTTCCAGGCAAAACTTTAGATGAAAGGTAATGAGCACAGCTACCAAAACCTGACGGTCTCTGTT
TCTCTGCAGCTGCGCGTATTCAAGCTGGCCAAATCCTGGCCCACCTTAAACACACTCATCAAGATCATCG
GAAACTCAGTGGGGGCACTGGGGAACCTCACCATCATCCTGGCCATCATTGTCTTTGTCTTTGCTCTGGT
TGGCAAGCAGCTCCTAGGGGAAAACTACCGTAACAACCGAAAAAATATCTCCGCGCCCCATGAAGACTGG
CCCCGCTGGCACATGCACGACTTCTTCCACTCTTTCCTCATTGTCTTCCGTATCCTCTGTGGAGAGTGGA
TTGAGAACATGTGGGCCTGCATGGAAGTTGGCCAAAAATCCATATGCCTCATCCTTTTCTTGACGGTGAT
GGTGCTAGGGAACCTGGTGGTGAGTGGTGAACCCTGCTGGGGGCACTGTCTCCTGGGGAAAGGAGAAGGC
TTCTGGGTTATCTGAAAAAGCTTTGATCTGCATTTTTATGCCCAGATTGTACTTCTCTGGTTGAAAAGTC
TTCAAACTTTTCAAATTCATGTGGGATTCATTACAAGAAGTGTGCCTTGGGAGGCAGTCTGACTTGCAGG
AATGAGAACATATGATTAGAAAAGAAAGACCTGGTGTAGAATCTCTGCTCCACCATTTACTAGGTGGGTG
ACCCCAGGCAAGAGACTTAATCTCCTCAAGATCTAATTTCCTTAAAATCAAGAAAATAATGCTTACTCAT
GGGTTTACTGGGAGAATTAAATGACAGGAGATGACATATGCAAATTGCCTAATGCAATGTCTGGGCTGAG
GCAGGAACTCAATAAACGGTGTCTATTAGCTGTTAAAGGAAAAATGTCCTTCCCCAATCTGAGGGGGCTC
AGAAAACATTTTTATTCAGGGAAGTAAGAGCTGACTGATTTCTCCAAGCTGCACAGGTCCTAGCTGGGAG
CAGGGGGAGAGGCTGCAGGGAGGGGGCACAATGGGTCACGGACAACATCCAAGGCCACAGAAATAATAAC
AAAATCTACTCCATGTTTGGAGGAAGCCCCTCTGGAGACTCACTTGCCAATAAGCCCCAATACCCCTTCT
CCGCCCCAGCCTGGCCCCCTGCCCTCAGCTTGTCTCCACCCACTCTGGTCATGGTGGGGTCCTTGCCCTC
CTGGGACTGACTGGTTGTGTGGGAGGATGCAGTCTTCCAGAAGCCCCTTTCTTATTGCCCTGGACATCAA
CCAACTTGGGTTCTTTCCTCATGAGTTTTATAGACAGAAGAAAGCTAAACCAAGAAAAGGGAAGCAACAA
CAAATCCAGACTCTTTAGTCTTTTCCACCCTGGTGCCTGAGCCAGGGATGCCTCTGCCTGCATCCCAGAA
ACTCTGTTCTTCCAGGCTGAGCCCACCATCCCTGCAGAGCCACAGATGCCCCTCACCCTCAGACAGGAAG
TTTACCCCTCTGAGCACTTTTACATTTCTCATATCATTTTGTCTTCACAACCGCTCTTCATCTGCTCTCA
AGTGAAGAACTGGAAGCAGCAAGATGTGCCCAGGATTCCAGAGTCAGGAACAGAGCCCAGGGCTTTGGCA
CATGAGGGTGGTTGCAGGAAGGGCTGGTGCCTTCTCTCTGTTGTCCCTAAATGTCCCTCTGCTCAGCGGG
GGGCTCCTCCTTTATGGTGGAAAATATGCTTCCAAGCCTCGTCCACTGAGGCGGGTCACTCCAGGACATC
CCCAAACTTTCCCTCAACTGCTCTCAGAAAGTCCATTCCCCCAAAAGGGACAGCATCCCGGGTGGGCAGG
AAGAGGCCCAGGCCGTGGCTGTCTTGCCAGCCAGCTGCTAACCCGGTAGGCCAATGTGGGGCTCAGCTTA
GAGATTGATCAATCTTCCCTTCCATTCCTTCTCTTCAGGTGCTTAACCTGTTCATCGCCCTGCTATTGAA
CTCTTTCAGTGCTGACAACCTCACAGCCCCGGAGGACGATGGGGAGGTGAACAACCTGCAGGTGGCCCTG
GCACGGATCCAGGTCTTTGGCCATCGTACCAAACAGGCTCTTTGCAGCTTCTTCAGCAGGTCCTGCCCAT
TCCCCCAGCCCAAGGCAGAGCCTGAGCTGGTGGTGAAACTCCCACTCTCCAGCTCCAAGGCTGAGAACCA
CATTGCTGCCAACACTGCCAGGGGGAGCTCTGGAGGGCTCCAAGCTCCCAGAGGCCCCAGGGATGAGCAC
AGTGACTTCATCGCTAATCCGACTGTGTGGGTCTCTGTGCCCATTGCTGAGGGTGAATCTGATCTTGATG
ACTTGGAGGATGATGGTGGGGAAGATGCTCAGAGCTTCCAGCAGGAAGTGATCCCCAAAGGACAGGTAAG
AGTTATCCCCAAGGGACAGCCACAGGCAGTGGGGAGGGGCTTCAGGGCTTGAGTGACAAAGGGCAAAAGG
GAAATGCAGAGGAGACCTTGACATAAAGAAGTTTAAACCATGCCAAGCTTTCCAGAGGAGTTGGCGTTTG
AGCAGGGCCTGGAAGAAAAAGTCAGAATTCCCCAGGCAGACAAACAGGGAGTGGGGGGCAGCAGGTGAGC
AAAAGGCCACCTTGAGGTGCTTGGGGATTGTCTTTCACACAGCTGTCCCATGCGCTCTGCCCAACCCAGG
CTCTGTGCATGAGATGCTAATGAGATTTCTCTAAGAACAGGATGTTGTCCAAGAGCAAGGCTCATCTAAT
CTTTACCAGCATCCTAAGAAGTCCTGATTCTTATGTGACTCATTTAGCCAGCTTTTCCCCACAAGTGGTA
TCACAGGGAAATGACACGCTCCTTTTGGCACCAACTAATTTAAAGCACATTTGAGCCATTTGATAATATA
TATTTTTAAATACTTGGCACTATTCCTTACTACCTTAAGCTGGTATATCAACCCTCGCTAACCTCTTCTC
AGTAACTCAGGGCATTCACCTCTGCCCAACGTGGGAAGGCAGGGAACCCTGTGAGGCCTCAGGAACATGC
AGGGCTGCTGGCCAGGTTACTAAGTGGTCCAGAAAAGCTGTGCTTTGAGTCTGTAGCTCTCCCATAGCCT
TCTTCCCTGCAGTCGTCACTTGTGTTCCAGCAGCTGCTGTTATCAGCTGCCATATTTTACATGCCTTTGT
CTAGGTAACAGCAGAGAGCATGAACTCTGTGACAGCTGTCTGCCACTCAGAAGCATTTTCAGTTACTTTG
GGGCTCTTAGGGAGCTCCAGGTGGCCAAGTTCCCTACAATTTTGACCTCTGGACAAATAACAATGGTCAT
GGCTAAAATCTATTGAGCCAAGCACTTCGTGCAAAGTGTTTTACAAGCGCTAACATATTTAGTCATCAAA
ACAGCTGAATGATGCAGATCTAGCCTTTTCAATTTTGAAGCTGGGAAAAATGAAGATTAGAGAGGTTAAA
CAACTTGCCTAAGGTCACACATCTTATAATTAATGGAGCCCAAGGATAAAACATAGGTCTATCTAGCTAT
AGTCTTCATGCTCACTTCACACACGTGTATGTATATATGTATGTACATGAGTTGCATATATGAAACTCTT
GGCTCTGGCAGCCTGTGATGTGCAAGATCCCTTCAAATTTAGAACTGGTCATCTAAGGGTGGGGCCAGGC
ACCCAGTTGGGACCCTCTCTCACTAGCAGGAGCAGCTGCAGCAAGTCGAGAGGTGTGGGGACCACCTGAC
ACCCAGGAGCCCAGGCACTGGAACATCTTCTGAGGACCTGGCTCCATCCCTGGGTGAGACGTGGAAAGAT
GAGTCTGTTCCTCAGGTCCCTGCTGAGGTAGGTATGTCAGCTTCCTGGAGAGGTTGGACAGCCAGCCAGA
CACTGAGCAGTGGGCTGGTAGAGGCTGGAGGGTGGCGGTGAAGGTGGGGTATTCCAGACTGCACTCAATC
ACATAACCTCATTTCTTATCCTGGGAATTTTTAAAGTCCCTCCCAACCTCCGGCTAATCCGCACAATTTC
AACTTTCACCATATACCATTGCCACAATCTCACTCCAGGGTGGTTCTAGGTATAGAAAGAAAGAGTGAGA
CTCCACAAGCTGTGGGACCTAAGATCAGTTCCCAAACCTCTCTGAGCCGCCATTTATTCATCTATGAAAC
TGAATGGTAATGCCTACTTGGTAGGATTATTTTGAAGCTTAAATGAGATGCTGCTTGTCAAATGTTTAGG
TCCTGCCTGAGACAAAGTAAGTGCCCAGGAAATGACAGCCAAGAAAAAAGGAAACGAAAGACAACGCACA
AAGAAAGTCAAATATCTTTCAGAGCCAGCAAATAAGAGTTGGAGGTGCTCACAGATGAAGAAGTTTTCCC
TTGTGATTCTCCCCCACTTTCTTTTGTCCACATCATTTTCTCTAGGCAAAAGTAGCCTGGGGCTAGGGAG
AGGCTCTCTATAGGTGAGTAGTGAAGTGACAGCCTCACAGAGACTGAGCTGGAAGATTAGAGGTTTAGGA
TTGTCTTATACAAGGTAAAAATAAATGTGGTTTCACTTACAAATCTTGAAGGAAACAAGCTATAGGGAAA
GAGGCATCTATAAAAGTTTAGCATCTTTAATAACACTGCCTTCAATTCAAGAATTTAATTTGACACCTAG
CCACTGAGTGCTGACTTTGTGACAGGCACTGGGCTCAACAGAGATAAGGAAGTCATAATTCATCCCCAAA
GTTATCAAGCCCTTCAGCTTGAAGAATGTCAGCTACTGGCTGTGTGCCCTAGGCAAGGCTGACCACAGCA
GGGTAAGATAAGAGGGAGGGCTGGGACGGGATTTGGGGGCTGTGGGTGAACAGAAGCCGCTTGGTTCCCA
CTGGGTGAGGGCTGTCACATTCTGCACGTGGATGTCATGCTTCTTCCCAAATGTTCCAAGGACTCCCCTG
GCTAGTTGTCCCTACTCAATGTTTTCAGTACACAACTTCTGGGTGTTGGTTTTTTGTTTTTGTTTTCTTG
TCCATGACCCCGGCTAGGCAGGAGGGGCCTGACAGCGACAAATGGGGACCCGCCCCACCATTTGGGTCCA
TTAGCAAAGCTATCAGGTTTACTAATATGAGAGCAACTTCAACCCCTAATTGAATTCATCTGGAGACATT
ATAGGGCTGGAGCTGCCAGGAGGGCAGCAGGCCCCTGCAGCATTACTCCATGATTAAATATTCAAGCCCA
GTGAATGCTGAGAGTCGTTATGGGTGTAATTACGGTGTCTCGGGCCATTGCCAGCACTCCATCAGGGGCC
CCGAAGGTTTACAGTCTCACACAGCTAAGCCTCTGGGGCTCCAGGGAGAAGAGCAGTGTTCCAGGCCTCA
GCTGGCGAGGCACCAAACATGGGTGCAGTTGAACCTGCAGTGACCGTGTGCAATTCCCCGGGCCCCGCGG
AGAGACGAGTTCACTGCCCTGGGCTTCTCCCCTGCAGGGAGTGGACGACACAAGCTCCTCTGAGGGCAGC
ACGGTGGACTGCCTAGATCCTGAGGAAATCCTGAGGAAGATCCCTGAGCTGGCAGATGACCTGGAAGAAC
CAGATGACTGCTTCACAGAAGGTGAAGGGACAGCCACAGGCCCCCTCACACTGATGTTAATTAGGGCCAG
AATCCAGTTGAGCGAACACTCACCTGGGATCCACAGAAGACAAACAAGCTGTGCCCACTTGCTGCCTCAC
TCCCAGGCTTGAGGGCGCCCGCATCAGACACACATGCCCCAGGTGGCAGCAGCCTGGCCGTGGTATCTGC
ATCTCATGCCCTATCAGACACTACGGAGAGACGTGGGCCTTCCTCTTTACCCTCAAAAACCCCTCCGTGA
GTTCTAAACCAGCCCTCTTGAGTTGAAAGGGGGAAACGTAGTCACCACCATAAACACCACTGAAAACTCT
TGATTTGGACCATGAAGGACCCAGAGGAAGGCCATGATGTATCTGCTTCCCTCAATCTTTTGTTTAGCTC
TCAAAAGCAGAACCTGTGTCTTAGTCATCTTTCCTCTCAGTGTTTAGAACAGTGAGGCACATTACATTCT
CAATAAATATTTGTGCAATGAATGAATCAAGAGTCCATTCTATAAAGGGAATTGTGGCCTCAATGCTTTC
TAAAAATACAAAGAAAAATTTTTTAAATACTTGAGTTTTTATTAATAGTTTTATTTGACCCTGGGCTGGG
CTGAGCTGGGCTGGACCCAATCCCCATCCCAGCCACGTCGTGGCCCTCAGTTCAGCTTGGGCTTGGAGCA
TATCCCCCTCAGGCACCCCCAGGCTCATACGCCGTAGCTTGTCTGTTTTCCAGGTATAGGTGGGCAGAAG
GACAGGGACCACTGGTGTCATCTGTAAGAAGGATATGGCTAGGAAAAACATCAGAGTGGGCAAGACTGAT
GATGGAGAGGAACACCTGTTGGTCTGGATTGAGGGACTCCCTGGAAGGGACCCCTCTTCACCCATATTCC
AGGAAAGAATTTTTTTCCAAGGGCATGACAAGTGGGTTTCCTAGCAGAATGGTTTCTCTGCAGCAGGAGC
AGAAATTATCCCCTTTGCCCACTGGCCATCACCCACCATCAGTCACCTCTCCCCACAGGATGCATTCGCC
ACTGTCCCTGCTGCAAACTGGATACCACCAAGAGTCCATGGGATGTGGGCTGGCAGGTGCGCAAGACTTG
CTACCGTATCGTGGAGCACAGCTGGTTTGAGAGCTTCATCATCTTCATGATCCTGCTCAGCAGTGGATCT
CTGGTAAGGGGAGGCATAGTCCCTGCCCCCAAATCCTCCAGATAGGAATCTCCTACAATCCAAAGGCCCT
GGAGCTGCATCAGTGGGTTCTCCTGACTACTGAGCTGAAAGTCCCCAACCTTGTCACTAGAAGGAGAGCT
GCAGCCCAGGGCGAGGGCCCCTCTTAGCAGCAGAGCATGGGAGGTGTTCAAGGTGCCCGTGCCATGGCCT
GCCAAACCTGGCTGCAAGCATTTGAGTGAGTGAGTGTATGCCTCCCACTATATAGATGGGGAAACTGACA
CTCAAAATGTTTAAAAGGCTTGTCCAAGGTCACCCAACTTGTCAGTAGCAAAACCCAGCTTTGCCCTTAT
TTAATCCCGAAGCCCCTGGCTTCCCACCCTCTGACCTCCCCAACACACGGCCTTCTCTGAAGGCCTACAA
TACAAAAAGGACTATCTTTCCTTATCAAACCACTTCCAAATGCTACTAATTACTCCTTTAAAGCTCACAG
AAAATCATTACAATAGCACTTCTCAAAATTTAATCTCCCAGGAGCTTGTTCAAATGCAGATTCTCATTCC
ATAAATCTGTGGCAGGGCCTGAGAATTTGCATTTCTAACAAGCTCCCAGGTGATGCTTGCACTGCCCTCA
GTGACCCACACTTTGAATAACAAGGCCTTATGGGAATAGAGATTCAGAGTTGGGAGACATCTCTAAGGCC
CTGGCTGAATACTGGGCTTTGTAGAGCAAAGAAGGAAGGTTCTCTCTGTACTGTGGCTTAAGCTCCTTAC
ACAATCTCAACTACTCCGCCAATAGACCAAGGTAGACATAGACACAGGAAGGAACTTAACATCCTCAGTT
GTTTGCATTTTCTAAATGAGGAAGTCAAAGCCCAAAGGCATGAGGGGCTTCAGGTAATTAGTTGAAGGGC
TGGGAATAGAGAGTTCAGGGTGGCAGCTAATTAGTTGAAGGGCTAGGACTACAGTCCCGCTCTTCCGATT
CACGGCTGCAGTGTTTTCACTCATCTTGCGCCTCATGTGTCATTTACTCTCTAAAAACAACCTACTAAAT
ATCTAGGCCTCCATCTAAATGCCAGATTGTCCAGGAAGGGTGACAAAACTGCTCGGTGCATGGCCTCAGC
TTTAGGGAGGAGCACAGGTGCGGGACGAGGCAGGATCTGAGAGGTGGGAGGAAAACTAGGGTGGCAATGA
CGCAAAGCCAAGGGAGAAGAGAGTTTCGGGGCAGGGACTGGTCTGCCATCCTGGGTGGAGGGGGAACTGT
GTGACAGCGGCTGCAGGTGCCCAGTGCCCTGGCACAGGGCAGTGATTGTCCACACGGACCTCAGCACAGT
GTAGAGGCCAAATTGCAGGGGCAAGAGAGGGCTGGGCAGTGCTTTCTCCACACCTATGCTCTTCTCCTTC
CAGTCCTGCTGGATTAAAGTAATAACCACATGTGCTGTTGGCCAGGTGCTCTCCTCACTACGTCATTGGC
TTGTGGGCCTTCTCTCCTCATTTTGCTTCTCTGCTCTCCCTTGCTCCCTCCCTTCTCTCTCTAGCCCCCT
TCTCTTCACACCCCGGTCCACCCCCCACATTCCCAGGAGGAAGCTCAGACCTGCAGCACTGCGCCAGCCC
AGCCCGCCCCCTGCAGCTCTCCCTTCCCTCCCATCTCCTTCCCCTGGGACTGAACAGGCTGGCACCAAAT
TTAGCCTTCTTTAGGCTTTCACTTTGAGTACCTTTTAAAAATAACATGCATTTTCATCTGACAAAGTAAA
GTACCAAAAAAATACTTGGAAATTATAGAAAAAGGTAAAGAAAAAAAAATCATCCATTTCCCTATCACTC
AAAGAGAAACTCACTTGAAAACATGCTGGTATACCTCTTTCTCATCTTGCTTATGCATAAAATATACACA
TATTACAAAAATAAAACCACATTTATGCATTCTATAACCTCAGTACATTTTTCTCAGTAAATAGTCTCAT
ATTATCTCATTTAATGGCTATAACATATTCCTTTATAATTTACTCAACCAATCTCTATGTTTACTTCTAA
TACACATTGAATATTCCCTGTGTGTACTCCCTTAGAGACGTTGGATAATCTCCCATCTTAGTCCTCGTCC
TAGACACAGAATCGGAAGCTGAGAAAAGATGTGGGTTCAGTTGAAATCCGGCTTCAGCTGATCCCATGGA
AAGCCCTGAAGCACAAAGGACACCAAAGAGCTGTCCCACCTTGAGGCAAAGAAGACAGCCTTGTAAATCC
CCCTGTCACTGTCCCTGGCTGAGGGCTGCCCCAGCGGGGAGGATAAGCATTTCCCAAGGTGGACACAGTT
TCCTCCAGCCCAGCGAAGCCATCAGGAAGGGGGTGTCTGCATCCATGAACTATTAGCAGCCGACACTCCC
AGCAGATGAAGGATAGGGGCACTGGCCCCTTAAAGAGGACCTGAGTGGGCACCAACAGCCTCCAGTGTCC
TTAGGATTAGTGCTTGAAGGAGGAATTGCTAGGTTCCTGAAACCTCTGAGTCACATTGCCAATTTGGCTT
CACACAGGACTGGCCAGGTTTTCACTCCTGCCAGTCACTTGTAAGAGTGGCCGGGCGCGGTGGCTCACGC
CTGTAATCCCAGCACTTTGGGAGGCTGAGGCGGGCGGATCATGAGGTCAGGAGATCGAGACCATCCCGGC
TAAAACGGTGAAACCCCGTCTCTACTAAAAATACAAAAAATTAGCCGGGCGTAGTGGCGGGCGCCTGTAG
TCCCAGCTACTTGGGAGGCTGAGGCAGGAGAATGGCGTGAACCTGGGAGGCAGAGCTTGCAGTGAGCCGA
GATCCCGCTACTGCACTCCAGCCTGGGCGACAGAGCGAGACTCCGTCTCAAAAAAAAAAAAAAAAAAAAA
AAAAAAAAAAAAAAAAAAAAAAAGAGTGGCAGCCCCCCACAGCCACAGCATCCTAGGGTTTCCCCTATTC
TTTTCCATCTCCACCAGTCTGATAAAAGAAAAACAGCATCTTCTTTTAATTTGAATTTCCTTGATTACCA
GTGAGATAGAACATTCTTTTTCAGAGGTGTGTTGGCGATTCTTACTTTCATTTTTGTAAACTGCCTGTGA
ACATCCGTTGATGCTTTTATTTTTAAACAAAGTCACTGTTAGCATCCCTTTGGTTCACTTCCAAAATCCA
ATTTGAGAAGGTTTTCTTTCTCCCTTGCATTCAGCATCCCTGCAAGTATCAGCAGGGCTGTTTGTCCCCA
TTGGCCCTGAAGAAGTCATGCTTTGGATCTCACAGTACCTCCTCCACTATCCCACTTTACCTCCCCACAG
CAAACAAAATGCTACTGGCAAGCTGACTACCAAATCCCCTTCCAGGGACTGTGGTCAGGGCTGGCTGGGT
CCTTCCCTATCAGTCTGGGCCCCAGTGCTGTGGGTCCAGGCAGCCTGCCATTCTGCATCCTTTCTTCCTT
CCAGGCCTTTGAAGACTATTACCTGGACCAGAAGCCCACGGTGAAAGCTTTGCTGGAGTACACTGACAGG
GTCTTCACCTTTATCTTTGTGTTCGAGATGCTGCTTAAGTGGGTGGCCTATGGCTTCAAAAAGTACTTCA
CCAATGCCTGGTGCTGGCTGGACTTCCTCATTGTGAATGTGAGTGGCTCAGTGGGAACACGTGGGGTTTG
GGGTCCTCCCCTCTGACTCCTGGCTACAGCTAGGGCAAAGGAAGCTCAGAGACCTGGCCCTCAAGGGCTA
CTCCAAAGAAAAGTTTTGAGCATTTAGTGAGGAGTCTGAGAATAGCCCTGACACCCATGAGTGGTAAAGT
CCAGGCAGTATGTGACTGTGACACAATAGTGACTGTTAGGTGAGTTATTAGGTCTCCATTCAGAAAGATC
CCTGCAATCCTCTGCATTTTTACATCCAAAGTGAGATGACACATGCAGCTCTCAGAGGTCTTATGGCCCT
GCATCCATAGATGGGTCTTCTGGACCTTGGGGAGCCATGAACTGTGAGCACAGAGAATCTTCCCACCTGC
GGTTCTGTGGTCCTCACACCTGGCTGCACTTGAGAATCACCTGGGGAGCTATGAAATATGCAGTGACCGA
CCACCACTACCCCAGAGATTCTGATCTTATTGGTCTTGGGTGGTGCCCAGGCACCTGTATTTTATAAATG
CTCCCAGGCATTTCCAAGGTGCAGCCATGGCTTAGAACCACTGAGCTTATACTGCTGCCTCCTCCCCAGT
GGCATTGAAAAGAACAGAGGGCGCATTTGGAGAGGTAATGAGCTGGAGTTCATGTGTTCACACTCATGGT
AACCTGTGGCCAGTAATCCCTGCATTAGAGAATTGACTGTGTGCTTCTCACACATCAGACATGTGATTCC
TCAGTCATCTCTTCCTCATCTTAACCACCACATTGCACTATCACTGTCTAGGCCACTACCTTCTTTGCTT
TGCAGCATGGTTAACTGAGTTATCTGTTCACTGCCGCATAGCTGAGGGTTTCTGCAAGCAGGGTGAGGCA
TGTGCTGTGTTGGTCTCTGCCTGCCCCACTCAAGCAGCTGCCAGCAGCAAGGTCTGCCCAGCAGAGGTGT
CAGGAAATGTGTCTGTATTTGGTGCTTGCCTCCTGAAGCTGGATGAGGTTCAGCACAGAACATTCTGTCC
AGCCTCCTGTGCTGGCAGGTCCTCCATAGCAGGCCTGTGCATTCAATTCTCTTCTTCCCTGATGCCAATG
CACTAGCAGCTTCAGGCCACTGTACCACAAGGATTAGCTGTGAGGCCTGAAATCTGAGTCATGGGTGATA
ATTCTGTGCATATGAAATAATTATTCATAAATGAAAGGAAAATTATTTATTCCGGTGTCCCACCCCTTTG
GGGGCACAGTCACTCCTGCTTATGTTGATCTCACCAGTCTCAGTCCTAATCACCTTAAACGGTCATGCAC
TCATTTCATCCATCTTCCTACTGATTCTTCCTTCCAACCATCCATCCTTCCTTCCATACATTTCACCTTT
TGTATCCAACCATTCAGTCTTCCTTTTTCCTTCCTCCCAAACATCCATCCATCTTTATTTCTTCCAGCTG
TACATCTTCATCAGTCCTCCCCTTCCATCCATCCATTCCCATTCTTCTTTCCCTCTTCTCTTCTGCCCAA
TCTTTTATTCTAAACTTCCTTCCATCTATTCATCATCCTTAACCCTTATATCTTTCTATCTTTTGATTCA
TTCTTCTATCCAACTGTCCAATCATCTAACATTCTGTTCACTCATTCACGCATCTTCCATCCAAGCATGC
TTCTACTCACTCATCCTTCTATCCACTCATTCTTCTATCTTTCCATTCTTGTTTCAACCTATCCATCTAT
TCTTCCTTTCTTCCACTTATCTGTTCATTCATCCCTTTCCCTTCCATTCATCATCCATTCATCCATCCTA
TCAACCTTCCAGTCATTCACTAATTTATTCCATTCATCTATTCTGTCCAGCTCCCCATCTTTCCATTCAT
GCATTAGTCATTTTACCCAGCCATCCATCCTTCTCTTATCCACCTATCTCATCTATCCATTCACTATTCC
CTGCTTTCATACTTTTCCTTTCTTGTTTGTTTCTCCCATCCTTCCATTCAACTATCTATCCTATCCATCT
TTTTGTGTCTGTTATCTTTTAAAATTTTAAAAAATATATTTTATTGTATATATTTGTATTAGTCTATTTT
CATGCTGCTGATAAAGACATACCTAAGACTGGGCAATTTATAAAAGAAAGAGGTTTAATTGGACTTACAG
CTTCACATGGCTGGGGAAGCCTTACAATCATGGCAAAAGGAAAGGAGGTGCGAGTCACGTCTTATGTGGA
TGGCAGCAGGCAAAGAGAGAGGTTGGGCAGGGAAACTCCCCCTTATAATACCGTCACATCTCATGAAACT
TATTCACTATCATGAGAACAGCATGGGAAAGACCTGCTCCCATGATTCACTTACCTCCCACAGGGTCCCT
CCCCTCCCACAACACATGGGAATTCAAGATAAGATTTGGGTGGGGACACAGCCAAACCATATCAATTTTT
AAGGGATACAAAATGATGTTATAAGATATATATATATATAAATGATTACTATAGTGGAACATATTAACAT
ATCCATCATCTCACATAGTTACCCATTTCTCCCCCTGTGGCAAGAGCAGCTATAATTACTCAGCGAGCAA
AAATACTGAAAACAATATGCTATTATTAACTATAGTATTCATGTTATACATTAAATCTATCCTATTTATC
ATTGTATCCTTTAATTCATCCCTCTATGCAATCACCCTTCAACTTTTCATCCAGCTGTACTTCTTTCTTT
CTTTCCTTCTAGCCATCCCAATGATCCTATTTTTCTTCCATTTGCTATACATCTAACTATTGAATACAGG
ATTGTCTTATGAAGTGCTTTGTAAGAAACAGAAGCACAGAATAAATACTCACTATCTCCAGGATCTTACA
CTTTAGTCTTTCAGCAGACAAGACACACATGGAAATAAATGTATTATAAGGGTGAATGTGTTGAGCACTG
CTGGGGGCAATTAGTATCTTGGAGACACAGAGACATGAGCCTTCTTCCACTCTTGGGGTGGGGTGGTTGA
GAATTCTTCCTATGGGAGGTGGCATTAGATGTGGGGCATAGAAATGAGCAGGACCAAGGAGCTGCGAGAG
GACTGGGGAAGGCCCGTCTTAGCTAAAGGCTGACCTTGAACAAAGGCACAGACACAAGTATGCTCAGGGA
ACAATTTGATTTGGGTAGAATGTGTGGGTGAAATATGACTGGAAAAGTAGGTTGTGACCATATGACACAG
GCTACAGAGTATGGAGTTTATCCTGAGGGTGATAAAGTACCAGTGATGTTTGGGAGTAAGAGAAAGACAT
GATCATAATCAGATTTGCAGTGGAGAATAGATGCATGGTGCAGTACTAGGGCCTGGAGACTACTTGGGAG
GCTAGTGGGCTGAGCAGTGGGAATGGAGGTGGGCTTGGGTGGACAGACTTTGCTGGAGAGGCACAGCACC
TGTCTGCCCCAGCTGCAGATTATCCATGTGAGGCAGACTTCCCAGGGGGCTGAGCAAAAGACTGAATAGG
GTTCAGCATGGAACTGGCCCCAAACCTCCCTACCCTCATGCACCCAGGCAGCTGGGTCTCACCTGCTGAT
GTCTGGAGCCTTGCATGAATGAAGGAAGAGGGTCCCTTCCATTCCGGTTCTTTGTCCTGGTGCAATAAGC
CACTCATCCAAAGAATGGACATTGCTTGGGGCTCTGCCACTCAAGTATAAAGCCCTTCTTTTCTGAAGCT
CCTGTGTGCATCCCACCTAACTGCCAGGATAAGCTGAGCCTGAATTCAGCCCACCATCTGTGGTGTGGCT
AAGATCTCACAGGACAGGGTCATAGCCCGTATCTATGACATCTCCCTGGCACCTTCAAAGTTCCTAGCAG
GGGAACTTTTTTCATGTAAGTTTCTGTCCTTTGCAGTCTTTGGACCACCACTTAAAGAAAACTGACAGAC
CAAAAGGGGCTCACAAGTCAGTCATTATATCTCAGATATCTATTGAAGCTAGATCTCATGGACCCATACC
AGAAACTTTTTGAAGAACTGCAGAGCTATCAGAACTTTGGAATTCTCTTAGTCTGACTTTTTCATTTTAC
AAAGGGTGAAAGTACCCTGGACTAGCCAGGCACTAACCCAAGCTCACAAAAAATCAGGGTTCAAATGGGA
ATTAGACCCCACCCAAACCACTATTCTTTCCACCCCACTTCTCTGAGCAAATATAATTCAGATTACTATA
TTCCTCAGAATAGACTCTCAGCATAGCCCCTAGGAGGGAACATCATGTCTCCAAGTCTCTGAAAATCAGT
TTCAAGGTTGTTGAGGCCTGACTGCTGCCCCTAGAAGGAAAAGGAAGAGTTGATTTAATACACTGGAAGC
TTGGCCACCTACGTACCAGAGGTTGGTGATGTTAGAGACTGAGTTTTTCTTGCCCAGATTGTTTAAGAGA
GTGGTGAATAAAATTTTTAATAAAAGGGAAGAGACAAAACTCTTTCTAGACCTCTCATTGGAGTTCCGAA
TAAGGTGGGAGCTAGGATGATGGCTTAGACGTGTAGGAATGGGAGGCTGGGCAGGGAGTTCCTGTCTCCT
TCCACGAGGACTGCCTTTCTGGAAACCTAGAGTGATGTTTCTGCCCTCTCCTGCACTCAGATCTCACTGA
TAAGTCTCACAGCGAAGATTCTGGAATATTCTGAAGTGGCTCCCATCAAAGCCCTTCGAACCCTTCGCGC
TCTGCGGCCACTGCGGGCTCTTTCTCGATTTGAAGGCATGCGGGTAAGTCTGATCTCAAAACTTCTCTCA
TCTGGCCAGGCACGGTGGTTCATGCCTGTAATCCCAGCACTTTGGGAGGCTGAGGCGGGCGGATCACCTG
AGGTTAGGAGTTCAAAACCACCCTGGCCAACATGCCGAAACCCTGTCTCTGCTAAAAGTACAAAAATCAC
TACCACCACGAGGTGTGGTGGTAGTGAGCCGAGATTGTGCCATTATACTCCAGCCTGGGTGACAGAGCGA
GACTCCGCCTCAAAAAACAAACAAAAAACTTCTCTGGTCTGCAGGTTTTGGTAACCCTTCCCCCTTTATG
CCCTCCATCCCACCAACCCTGAGCTCCGGGGCCCCACCTCCAAAGGTAGGCTCTGAGTCTTCACTTCCTG
CTCTGACTTTGGTGGTGCTGCATGTCCTAACTCTCTTAGTTCTCTTTCAAGTCTTTTTGTGGGCCCATCT
TCCCTTGTCACCCATAAATATTACAGCCACCAGGACCCAGACCTCACCCCCTTTGCTCTTAGTGAAGTAA
TCCTGAATCATCGCTCCACAAGCATCTATTATCTCAATCACCCAACTCTGCCTCTTATTGCCAACATCTC
CAGAACATCCCAAGTTCTCTGTCTTTCCATGACAAATGAGTCTTTCCTCTAATACTCCATGAGTGACATC
ATCAGCTCATTACCTCAGCTGCCACCCACTGGAATGCTTCTTCTCTCCTACTTAATGCAATCATCAGGTC
CTGTAAATTTTTCCCTTCCATCCACCCCTTTCCTTATCCACCACTGTCAGCCTCCACCTTGCCCTAACTG
CCTCCGTCAGGACAGAATGCTCACAGCCAGAGCCTTGTCTTAGAGATTGCTGATTGACACCCACCTCTTC
CTCATAATTACAAATGGCTCCCCAGCAGAAAGTAAGGTTTTGCTTTGCTCCACTATCATTGGCTGGAAGT
AGTCATCATAGCTTCAAAAACAAAAGTGGGCTTTTGAAATTAACTTGTACCTTTACAAACACTAGGACCC
GAAGATTGAAATAGTTTGAAATTTATTCTATTTTGTATCTAATAGATTCTCATTTGCTTATTTACAAATA
AGCTATTTTGTGAATATGGATTTAGGTTTTCACTGGTTCTTGACCACGGCATACTTTAAATTGTATTTAA
ATTGTGTTTGTGTTTTATGTATGGTATTTAAGGAGAATAAGTCAAGGTTCTAGAAGTCTGTATGTGCAAG
TATAATACACACACTCACACTTCTAAATGAGTGCAGTATGCTTGTAAACTCATCCATCTTCTCACTGCAA
CCCACCCTGAATGAGGTTAAGGGAAGAAGGTCTAAATCCATCTTCAGCTGCCCAATGTCTCTCAGTCTTC
TGAGTCTGAATTATTGCACTAATTAACCCACTTCTCTTGCTTCTTCCAAAGGCCACTGGCTCATGAAGCA
GCCTGAATGAGTATAGAAAGAGATAGAATCCAGCAGAGGGGGTGGGGCATTGGGAGGTTCCAGGTCTTTC
TCTATCTCCCACCAGGTGGTGGTGGATGCCCTGGTGGGCGCCATCCCATCCATCATGAATGTCCTCCTCG
TCTGCCTCATCTTCTGGCTCATCTTCAGCATCATGGGTGTGAACCTCTTCGCAGGGAAGTTTTGGAGGTG
CATCAACTATACCGATGGAGAGTTTTCCCTTGTACCTTTGTCGATTGTGAATAACAAGTCTGACTGCAAG
ATTCAAAACTCCACTGGCAGCTTCTTCTGGGTCAATGTGAAAGTCAACTTTGATAATGTTGCAATGGGTT
ACCTTGCACTTCTGCAGGTGGTGAGTCAACCATGAGATCAACCATATCTCTTGCTTTGGCCGCTCAATAA
AATGGAGAGTCTAGGTTCCCAGAAGATGCTATGTGGGGACTTTGCAAGGAGTAGCACTCAGTGTGAACTG
AGGTCATTCTCTATTCCTACAGACTACACCATGAGGCTAGAGGGCCTTCCTAGTGAATATGATATCACAG
TGGTGCAATTACACAAGTGTGGGCTAGTAGAATAGCATAAAGGGATGGGCCTGCCATGGGATTATCAGGT
GGTGAGTTAAAACACAGAACCTGTACAAAATAGACACGGCATAGCCACATGTTCAGTCTTCAACTTCACA
TCATTTCCTAAATGGTAATAAATTATGTCTCATTAGGTCTATTGTAATGATATATAATGTTGGTGATAAT
GACAGTCAAACACATTCATCCTGTCTTTCTTTCTTTCATCATCCAGTCAATCACTCATTTCTTGCCTTCA
TCTACCTGTTCATTCAGTCCTCTGTTCATTCAAGTGTTAATACAATTACTTATACATTTGTCATGCAGTT
ACTCTTGAGTGTCTACATTCCACCATTGGTTCTTGCATTTATTCAACAAATAATCTCTGAGTAGCTCTGG
TATGCCAGGCATGGTACTACTCAGCCCTGGGAGATACAGTCAGAACTGGGCTTGGACACTGATGCTGTAT
GAGAAACAGACTAATAGGTGAATTAAAACAGCTCATTAAGTGCTGTAACAGGCCTAGGAGTGTAAGAACT
AAGATGTCTTGCATGCTCTGAACATGCTGGAAGGCACACCCAGTTTCTAGACCTCAAGAATGCTAAAGCT
GAAGGCAGGAACATGGAGGTCTTCTATTCTCAACTTCTCCATCTGGAAGATATACCATTCTTACATCATG
TGCATAAGGTGGGAAAGTGGCTGAGAAAATGGCAGCCCTGAGAGGGGAAACCTTAGGCAAGTGCACAGAG
CAGACTTGTAGCAATTGGCACTAGAACCAGACACCTAGACACTCAGTCCTCAGTCCATACTACATTCTGA
AAGGCTCTGTGTCTTTAATGCTACTCTATTCTTAAACAGCCACATGTGGGATTACTTTAAATCATCATAT
GAGATTAGTCAAGTAACCCAAGGGAACTTTTCCTTTCATTCTTTCTGCACAAGCCCTTTCCAACACAGGA
CTCTCTCCTCTCTCACTGGGCCACCATGGCTTGGGAAGGGTCCAGTATAGATGGATGGGCAGACACTGAG
TGGCCTCTGCTCCTTGTCTCCAGGCAACCTTTAAAGGCTGGATGGACATTATGTATGCAGCTGTTGATTC
CCGGGAGGTGAGTCTCAGCCCACTGTCCCTACAGCCTTCCCCTCTGTGCATTCTACCCATATCAGTCTGC
ATTTGAAATGAAGTGACGATGACCCTGGTACTGGAAGAGGCGATCACCCAGCAACCTCACACTGTCCCTC
CTTGCAGCAGTGTCCAGCTGGCTCTCTGTTGAGTGTTTCAGCCACCGAGGCTTTCCCCCTTTCATCTACA
CTCCCCAGGCTTCTCGAGGTTTTCCTCTGTTCACTGTTGAGGAAAGCATCAGATAACCTCTAAGAGACAG
TTCATGTGAGCCACTCCCTGGACAAGGGTGGGAGCAAAGCAAGGGTAGTAGCTAAATCAAGGTGCATAGG
GAGGAATGAGGTAGAATCTCAGGAACTATCATAAAACCCAGGATGGGCCAGGCGCAGTGGTTCATGCCTG
TAATCCTAGCACTTTGGGAGGCCGAGGTGGGCAGATTGCCTGAGCTCAGGAGACCAGCCTGGGCAATACG
GTGAAACCCCATCTCTACTAAAATATGAAAGAAATTAGCAGGGCATGGCGGCTTGTGCCTGTAGTCCCAG
CTGCTCGGGAGACTGAGACAGGAGAATCTCTTGAACCCGGGAGGCAGAAGTTGCAGTGAGCTGAGATTGC
GTCACTGCACTCCAGCTTGGGCGAGACAGCGAGACTCAATCTCAAAAAAAAAGAAAAGAAAAGAAAAAGC
CAGGCTGGGGAGACACTATTGGGGAATGACCACTTCCTGCTGTGGCAGGCACTGTCAATTCCCTGCCACC
TCTTCCCTTAGCCCACTTCTGATTTCACCTGCAGGCTGCACTTCTGATTCTCTGTATAAAGACTCCAGGG
CTTAGGAGCTTGTGCAGCAACTTCTCCCCCAAGTACCAGGGATTTAATGTCCCTAGGGGCAACCCTTACC
AATAAGTTTTGGGAGTCTGAGGATAAATACTCCAGTTTCCCTGTCCCTTGGTGATATAATCCTGCATGTG
CTCTACACAGTTACTCAGAGGACCCCAGTAGGATTGCGCTTCAGTTGCCCACAATGATGCCCTGCTTAAC
AACATCCTCTTCACAGGCTCTTCTCCCATTCCCGCTTATTCTTCCCCACTCCCTCATTCCTGCTTCCTTG
GATCACCTCCCAGATAAATCACCTGCACCCAGGTCCATGTCTTAGGCTTGGCTTTCAGAGTGAACCTGAA
CTAAGACACCCACCCTCCTTCCCACTCTCCCTTGTCTGGTCACAGGTCAACATGCAACCCAAGTGGGAGG
ACAACGTGTACATGTATTTGTACTTTGTCATCTTCATCATTTTTGGAGGCTTCTTCACACTGAATCTCTT
TGTTGGGGTCATAATTGACAACTTCAATCAACAGAAAAAAAAGATAAGTGGTGCTCAGGAGTTTCTGGTT
TCTGCTGTAGTGGTAAGCCTTATATTTCCTGTTCCCTGCCCAGCTCTGGCCTCCTGACAAAAGCCAACAC
TACTCACAGCCCATCACTTTAATATCAGTGCTAACCCTTGCCCTGTGATTGCTGTTGCAGGAAGAGTTGC
ATTTCTCTCTGTCAGCAATGCCCCACTAACATCCTCACTCAGGATTCACATCTCAAAGGGATCTTGGTCT
TCCCAACTGGAACCAGGTGTGTTAGATGTGTTCCAAGTGAGCCCTGGGAGAATACAAGCAGGGAAAGAAA
GGGATCTAGAACTTGCTGACCTGTTGGGGGATATGATCCCCACAAAGGACTGAGTCAGGGCACCTCAGGA
AAGGCCATGGCCCAGCACTGGCAGAATCCCAACCCTCCTTGGGATGGGGAGGAAGGCTTGTGCCTCCAAG
GGTGCCGACTCTGATTAATAGGAATCGAATCCCAGATAACCTCTCATGTTTATAAGAAAATGTCAGGGCA
ATAAAGGTATAAAGTATTTTCCTATTATCCTTTCATTTAATGCCTTCCATACCCCTGAGGTAGATAGGCG
CTGTTCTAGATTTGCGTAACTGAGATCCCGAGAGAAAAGGGGCTACATGGATAAGTCACAGAGTACATGC
AAACCTGGGTTTGCATTCCTGACTCAACTACTTGTGGATGGCTCATGCTGGCAATATTTTAAACCCCTCT
GAGTCTCAGTTTTCTCATTCATTCAATGGGGCTAATAATACCTACTCTCTGGATTGTGGTGAGGGGTAAA
CAAAATCCTGAACAAAGATCACTGAGCATAAGACAACTTTGGTGGTGCAGTTTCTGGCACTGTAACAGCA
GAGGGGAGTGTGACTGGCAGCGCCTCAAGAGCACCAGATGCTGTTGCTATTAACAATTTAAAATATATAC
CTACTAGGGCAGGCATTCCACTTACAGGAAATCCTTGCCCAGGGTTCTGAGCATAAGAAGAGGACAGGAT
AGGTCTGGGACAAGACAGTGCTATTTAGTACCCTGGGGAGGTTTGAGATTATTGAAGCAATCATTTCTCT
CATTTTACAGATGAGAAAACTGAGGTCAAGAAACAGATTACAGAGGGAGATATTAGGTAACGGAAGGAGA
TGAGCTAGTGACCAGGCTGGGGTCACTGAGAAACTGCATCTACTGAATCCTTGGTCAGAGTTCTCAGCAT
GACCTCACCTGGAGCTCACCTACCCACTGCCCTTGCCCTTTCCTTCTCTCCCCCCATAAAGATGGTCCTC
CCAGGAAGCCCTTTGTGTCCACAGATCTTCTCAGATCTCCTTTCAAATTCCACAGATCTCAGCCCAGTTT
CCCAGCATTTGCAGGGACTAACTTACATAAACATGTTATTAAAATAGGTGGCTTTCAAATTATGTCAGGA
AGCTAAAGGTGAGAGTTTGAATGAAGAAAATCCAAAGGGCCCCTAAAGAAAAACTAATACCCAATCCTAA
GGGAGGAAAACATCTTCGTTGGTGCAGTTTCTGGCACTGTAACAGCAGAGGGCAGTGTGACTGGCAGGGC
CTCAAGAGAACCAGAGGTCCCTCTGAGACCAAGGACCCAAGGATAAGGCAGTAATTGTCCTCATCTCCTC
CTCTCCAGAAAAGATTTGAGGGCTTCTGTCCCTCCCAGAGTCTTTGCTTCAAGGTCCTCAGGAAATCTCT
TGCCCCCTGGGAGGGACTGTGACTCTAGAAGCCCAAAGAATTTTACTGGAATGAACAGGCTAGATCCAAG
GCTCAGATTTGAACCAACATGTACCTTCTCCTTGAGTCCTGTTTACCAACCCTCCCCACCCTTTATCCAG
CATCTCTGGATCCCTGGAGAACAGAAACTGGCTTTGCTTTAGAGACCCAGGCAGGGAGTCAGCAATGGAA
CTCAGTCCCACCGTTCCGACACTGGATGTGGCAAGACACTCCTTGATGCTGGGCTTGGTTTGTCTATCTG
CAGAGGAGGGGAGAAGGTTGATATGATCCTTTCTCTCCTGCCTGTTCTGATTTTGGTATCCGTAGGGGGT
TCCTCTTGCTTTAGTAACAGTGTGGCCCTCTCTGCACTTAGGGGGCCAGGACATCTTCATGACAGAGGAG
CAGAAGAAATACTACAATGCCATGAAGAAGTTGGGCTCCAAGAAGCCCCAGAAGCCCATCCCACGGCCCC
TGGTGAGCCCCAGAGGTTTCCTCTAGCACAGCCTGTCTGTGAATCCAACATGACAGTCTATGCCTGTGTC
AGGCAGGGGCCTGAGGGAGTGCTGTTTATGGAAGAGAAGATCTGGGTTGACGTTGGGATGAGGGTGATGA
GGGTTCCTAGGCCCAAGGAATGAGAAGTTCAAAAGAAGCAACTCTGGTCTTCTGGAGTAAACATACCTTT
TACAGCTATTTGAAATGAATCTGTTGGGGGAAGCACTGAGTTTCTCCAACAGTTTTAGAGTTCAAGTACA
GGATTGGAGGCAGGTTAAGCTAAGTGACAGTAAGGGTAGTTAGAACCCAGGGATTAAACTTGTGTGGTTA
TGTATCATGGTCTAGGTCGCAGTAGATCTCCAGTATCACCCTATATTCTACCCCACGCCTACAGGTATAG
AGAGGAAATGACAACAACCCAACTCCCCTGTCAGGATCCAAGATGCAGTCATGGTAGTGGTCAAGTGGCC
CAGTAGGAACCAAAAGTTATCCCACTCGAGTTTAAAGATTGGGCAAAAATTAGGGCCAGACTCTAGTTTC
ACTCCTTTTTTCATAAAGATCTCAAGAGACATGGCTTTGCGTTCCTCCATTAAATGTACTACCCATACCA
CACATGATATCACCTAAGTTCTTCTCAGTCTTCTGCCAGAAGATAGAAAACATCAAGCCTCTGAGAGGCC
CTAAGATAGCACTTTTTTGGGAATGGATGAACAGGTTTGGGGGTGAAGATGTTGATGTGTCAGAAATTAT
TGTTGATCGTCTTCAAAGAATTTCTGAGCCATCCACTCCAGTGAATGGAATTGGCCCTGTGTCCTTCTAC
ATAAACTTTCTGTGGCTAAGATGCAGCATTAGTGAAGAAAATCACAGGTATTGCTGATGACACAATTTCA
TTAGCCCTTCCCCACACAAGGGAAAAGTCCAAAGGACATTTAACAAAGGAACAAGCCTTTCCCTAAATGA
ATTATGGAGACATGCATTCTGTTAGCCAAGCTATTAGATTTTTTTAAATCAAGGTATAGGATTTGCATAA
TTTACTTCAAACTTTGTCAGAACAAAGTGAAAAACATTTACAAGAGAAAAAAGTGATGACTTTTGACACT
GTATTTTGAAATAGTGGTCATGAATTTATTACTAGTCTAGATTCTGATAAGAAGTATTACAATGGGGAGT
GGGGAGTGATAGGAGGTTCAGACCTCTGTAGGGGCACAGCTTTCTTCCTAAATCACCCATACAATCAAGA
TAACTGGATTTGAGTGAACCTGAGCAAAAGTCCTTAGGCTCATTGAGTATCCCTCCATCAACAAGTTCAG
TGGAGAGGAGCAGGCTCAAAATTAAAATTAGGTGAAAGGCTGAGTCCCAACCACATTCAAGTCATGGGTA
ATGAGAGGAGGACCCTAAGACTTACTGAGAAGCAAGCAGCTGAGTCCACACTGGCCTTTACTTGTGGCTG
GAGTAAGATTGAATCCACATTTGAGCCAGATGCCAAGGAGGTGTGTGCTAGGGACAACCCTTGGCAAAAA
CATGAAAGTTTTTGGCACCTTCCCCCACATACTACTCAATCTCTCTTTGACATCCCAGCTTCTTCCCTGT
CATCACCCAACCCCATAGGCTGCCAAAGAAAGCTTCCTCCCCTCAAGGAAGACAACATTCTTAGGACAAG
ATAAATCATATTTCAAAAAGGGACCCTGGAGAGGGCATCTACCTACAAAGGCATGACCCTAATGTTGAAA
TTCCAGGCAGTAAATAGGAGTGGGAGACGGAGTTGATGGTCAGGCAGGAAGACCTGCAACAAAATCACCA
TTTCTCCTAATTAAGCTGCAACTAAGATCCTCCGAGGATGGCACAGGGGGAGACCTCCACATGTGGAGGG
GAAGTGAAACCTCATAACCAATTCTGTTCCTGAGATTCTCCATCCTTTGAATGGAAGAAGAGTGGATTAG
TAATTCACTCTCAACCTTTTGCTAGCCTTAGTGGAGGAACGTGGCTCTCCCCTTAATATTCTGGCCCAAG
CAAGGTAAACACTCTTTCCTTTGTTCCCTAATTTCGCATAGCTGAGGCAAGGCTAGAACATTTAAACAAA
ACTTTGAAGTGGCAGGGATGCCAACAGAGAAATCAGCACAAAGCAGAGTCCAAGCAGCACCTGCAGATGG
CTGCACTCCAGGACCCATAGGTCTTGGACCATCCTAGCTCCTGACCAGCATCCAAATGGCAGGCAACATC
TTTGACACCCACATGCCCAGTGGCACAAGCCGGATCATCTTCCCTCTTCAAAGTCTAGAACCAAGAAAGA
AATCCAAGGCAAAAGGAAATTCCCACTCCCAACTAAGACCCTTCTTCCCAGGCCCAGCTTGCTTTTGGCC
AGATTATTGGCCCTGGAATGTCCACCTTCTCCCCTTAGGAGCCCCAAAGGTCAAGCTGGTCATACCAGCA
AAGCACTGACACAGGATTTCTGCCCCATCAGTGCATGGGTGCCATTTCATGACAGAGCTTACCTGGGACC
ACATAGGAGCCTCACACACCTCCTCTCACCTCTGATGATGCTCAGGCTCTAAATCTTGTTTCTTCCATAG
GAAAAGTTAAGGATTCCACAAGACTGGGAAATTTTATTCTTTAACATTCCCTGCTACAGAAATCAATAAA
AGCAGCTCTCACATACTCCACTTTATAGAAATATAATAGGCTCTAGAGAATATCACATAATCAATCACCC
ACTTCATCGTCTCTGAGAGCACCAAGGGTTGCAAAGGTATGATCCCTGACCGTGGGCTAATCCAAGAGCA
GCTAGCAAGCTACTCCATTCCCTAAAGTCAGAATATCCAGGGCAAACTTCACACCCAGCCACTTAACGAT
GCCGTTGCCTGCTCTGTTTAATTAGAAGTCATGTGACCATGAATGGGCCTGTATTTTTTCACCATTTTCA
GTGATGAAATGGAAGGGATTAGTGGCTCAGCAATCCCCCTCCTGCCCCAGGAAAGATGTGAGCCAGCAGT
TCTGCCATGAGCAAAGGCCACTTTGGTCTGATTCTTTGCTCTAACCCAACAGGATAGCAACCCATCCCCA
CCATGAAGCAGAAGGCAAGGATCAGCCTCATCACAGCACTTCCCCCTCTGCTTGCTGTGCAGCTCCCGTG
GGCTCCCTGGAAGGGATCAGACAGGTTCTTCTCTTCCTCCCTCATAGCCACTCATGCAGAGGCTTGCACA
TCCCATACATTCAGTATGTACCCTTGGTACAGAATTGAATTTAGTCAAATAAAGGGTGACTTTAAATTTT
CATTTGAGCTATGGTTAACTCAGAGCTGCCCATTCTGTCTTCCATTCATTTACAAATTTGCAGAGCATCT
GTTGCAGCCAGGCACTATGCAGACACCAGGGTTAGAGCAGTGAGCAAGACAGACAAAGTTCCTACTCTTA
CAGTTTACAGTCCAGTGACAGAGGCAAACAGTTGGCCAAAAGTTGCACATATATGTTAATTCCTGGCTGG
ATAAATTGTCAAGGGAGAAAAAATGGGGTGAAGAGCAAAGGATGCTCAAGACTGAGCCTCGAAGAACTCT
GATGTCTAAAAGTTGGGTAAAGGAGACTATAAAAGAGCATCACAAATGGCAAGAAAAGAAGCAGAAATGT
AAGGCATGGTGGAGACCAAGAAAGCAACATTTCAAGGTAGAAGGGTACTAGGCAATATCAGACAAGCGTC
CTTGGATCTAATATTATGGGAAGTCATTCATGATCTTAGTGAGAACAATCTCTGTCAGGATAGAGGCAAA
AGCTCTGTTGATGTGGGCTGAGGAGTGAATGAGGGACACTGATTGTACACAACTCTTTCAAGAAATTTTG
CTTTGAAGGTGGGAAGAGATCTAAGATGGTAGCTGGAGGGAGATGTAAGTTTCTGAAATATTCTGCTTTA
AGTTAGGAGAAACTAGGTCATTAGAGATTTGTGCAATAAACCAAGAGACAAAGAGTAATGCACATGGACA
TTAATGTTGCCTAGGATAATAGCCAAGAGTGGGCAAATATGGCCAAGAGCCTCACCCATCATTACTGAGG
CAGAGGGACCAAGAGTTTGGCAAGCGGCAGTAATGAAGAAGTGACAAAACTAGGTGGCAAAGCTTCAAAG
CAAGAAGGGGTTTTACAAGACACAGGAAGAGCAGTGGTGTGGAAGGGTCAGTGGTGAACAAGGAGAACGC
CAGGCCTAACCCCTGAGGTACATGGGGTAGGGGAGAATGGCAGCCTTTGAATAGGGGGAGATATGTGCAG
ATCAAGGCAGGTGAGCTCCATAGAGGTCAGGAGAAGTTTTGGGAGAAGGTCAAAGCGTGGGGAATGGGGT
TGGGATCAAGGACTCACAGAGCAGTAAGATGTTCACAGCACTAAAAGAAGAGAAGATCACAGTAGGAGCT
TTTTAAAGACACCATAAAAATAACCAGATATAAATGATCTCTTGGTTATCCAGGCCACTCTGCCTCCTTC
CAAGATGCAAACAACCAAGACTTGACCCTACGCCCTATACCTGCATTTGCTTAGGCATTTGCACAGCACC
AGGAACAGAGGGAACATTTCCAAGAATAAGTTTCCCCTTTGCACTTGACTTTACTCTCAGCAAAGTAGCT
CAAATTAATATTTCAAGCAGCTCTTGAACTCTTGCAATGTGCAGGGCACTGCAGGGAAACAAAGTTAAGG
AAGGTATAGCCCTTGTCCTCAAACAGCTCATTGTCTAGTAGAGAAGACGCATATACAAAATGCTAGAACT
CGAGGCTGACTGAAAAGTGGGATCCCCAAAGGGACAGGGTAGTGTGTTGTCACGTGACTAAGGGACACTC
CATGCAGTAAGTGGAGCTTAAAAGAGATGGATTACTCAAGACTGTTGGAATTTAGACAGCCAAGAGAGGA
GATAGGAGGGCATTGTGAATAGAGGCAAGGGCACAGACCAGGACAAAAGAAGGGCCTGGTGGCATCAAAG
GGCCAGGAGAGGTATTAAGGACTTGAACTGGGGCAATGAGGTAGAGTCAACTAAAAGTGAAATTTTGGAA
GAGGTAAGGATAGTATTTCGCTTCTGTTAAGATGTGGAGTGTGATGGAGACAAAACTCATCTACAGATAA
CACAGATGCCTGGTTTGTGCTGGCCCAAAGGCCCTGATGCCACTATCTCTTGTTTTCTCAGAACAAGTTC
CAGGGTTTTGTCTTTGACATCGTGACCAGACAAGCTTTTGACATCACCATCATGGTCCTCATCTGCCTCA
ACATGATCACCATGATGGTGGAGACTGATGACCAAAGTGAAGAAAAGACGAAAATTCTGGGCAAAATCAA
CCAGTTCTTTGTGGCCGTCTTCACAGGCGAATGTGTCATGAAGATGTTCGCTTTGAGGCAGTACTACTTC
ACAAATGGCTGGAATGTGTTTGACTTCATTGTGGTGGTTCTCTCCATTGCGAGTAAGTGGGCAGCCACGT
GGTGGGGAAGCCCCACCTCTGTTTGGGGGTCTTTGGGATGCTCTTCTAAAACCAAGGAAATAACCAACCA
ACTTTTTGAAATACGTGTTTATGTTAGAAGAACCCTGTATTTGCAGCTTTCCAAGAGTAGCAAGTTTTAA
ATGCCTTGGGCTGCTCTCTCAGTGAAATGAAGAGGTTCTCTTGGCCTTCTCCATGCTCACACGAAGTGTT
TTCAGGCACCTACTGTGTGCACAGCCCATGGCTAGAACCTCAGGGGCTGACAGGGAATAATCACTCCTCC
TGAGAACCTCAGACCGTAAGATGATGGAGTCTCCAGCAGACCTTACTTTTATGGTCTTGAAAGCAGGGGG
CAGGTGGTGTAATGTGAGATGAGTCTTAAAATCTGTTCTGGAAAAGGAGATGGATTGCCATCAAAGTGAG
GAAAGACTGGGAGGAGATTGTTCAAAGGAGATTCTCAAGTCTAGGGTCTGGAATTAAATCTCTACACAAC
TGTTCTATTAGGGAACATGAGTGATAGAAAAGTCAAGATATCAATGCCAAGAAATTAATACTGTTAAGGC
CCAGTCTGGGTGTTGTTGAATGACAGGCCCAGTATTTTGGGTCCAGTGGTGTTCAGAGAAAATTACTAGA
GTTAGAGGCTAACATGGGGAAATGGAGCTGCCTTGACTGAATACTTCCTAAGTTCTGAGCACTGAGCTGG
GCTCCATGAATGTTCACAGCAGCCCTGTCAAGCAGGTGTTATTATCCTTATTTTATACATTTAAAATTGA
GACTCAGAGATCAAGAATATTATGCAGGGTCACAGAGTTTCCAAGGGTTGAAGCTGAGTTTAGAACAGGC
TCCAATCTTCTAATGAACTTGCTGCCTGTCACCAACAGCCAAGAGACTGTCCCTGGGCTTATGGGTTAGC
TGAAGTTTGACCCTATAAAGGCCAAATCATGCTCACCAGCTTCACCTGGCATTATAAATGTATCTTGATT
GGTTATATCCTGACCTGCTTACTTTTCATGTCGGGGTGTAGCTGTAGATTTAATTTGCCACTGATTTAGT
AACAAGAGCTGAGAGTATCTAAATACAACTTAACAGCATTTCATCAACCTCCCCACCTTGGTGCTGAAAC
ACAGCCTTGGGTTGCGTGTATTTCCGGGGATTTCAGTTTGTTTTTGTTCTTGTTTTATTTTGATATTGCT
GCTCTGTTTTTTAACACTTTATTGAGGTATGATCCACATGTAAAAAGCTATACATGTTTAATGTACACAA
CTTTATGAGTTTGGAGATAAGTATACACCCATGAAACCATCACCACCATCAATGCCAGAAACATGTCCAT
AACCTCCAAATATTTCCTCCTGCCTATTTATTTATTTATTGGGTGCTAGGAACACTTAAGATTTATTCTC
TTGGCAAATTTTTAGGTAGACAACACGGTATTGCTAACTATAGGGACTATGCTATAAGTAGAGCTCTAGG
ATTTATTCATGTCACATAACTGGAAGTTTGTACACTTTGACAAGTACCTCCCCAATTTCCCCTCCTCCCA
GCTCCTGGAAACCACCATTCTATTCTCTGCTCCCATGAGTTTGACTATTTTAGATTCCTTATATAAATGC
AATCATGTCGTTTTTGTCCTTCTGTGTCTGGCTTATCTCACTTAGCATAATAGGAGTTTTTGTTGGTTGG
TTGGTTAGTTGTTTTCATTTTTGTTTTGTTTTGTTTGTCTTCCAAAATGAAAGATTCCAAAGGCTATTTT
GATTTTATATTAAAGAAGGGAGCTTGTTTGTGTATCAGTAATATAGAAATAAAACATAGCCAAGTACTTA
GTTCTATGATGGGGATATCTTAACTACCAGTTCCTGTGGCTTACAGGGTAGCACCATCCGCTGGGGCAGG
AATCCCTTATAGAGCTGGAAAATAAGCAGAAAAAACCCACATATCAATCTTTCTACCCTCTCTCCTTACC
TCCCTTTCTTCTTTCCCACCAACAAGAGGTGATTGAGTATCTAATTGTTACAAGTACGAGGAACCATTCT
CCCTTAAAATAAGATCCTGGAGCAAATTAAAATGTGCTGTAGACCAGTTGTCTGGCAGAAAATTTTACCA
GAGTATGTAGACAAATAATGTACCCTACACGATGGGAACATGTCCTAGTTAAACTGGAAATTCACAGAAG
GTCTTCCACAAAGATTTTATAATCCAAAATTATAATTATTTCCCAAGTCCCCTTCTACTTTCTTCCTCTT
TCCAACACAATATTGTGACCCATGTGCATAACAACAAAGAGCAAGTAGGGGAGAGGGCCCAGGGAACCTG
GCAAGGAGCGAGAGCTTGCTGCTACATCCCTCACCATTTTTCCTGTGAGATGTTGAACCCTCTCTTACCT
AAAGGCCTTGCCTCATCTTTGGTGGAGGCCTTTGGTACAAATAAGGCTCCCCAGGTCCCCAAGACCAAAA
ATCAAACATCTTCAAGTTAGACATCATATAGAGGGACATATACATTCTTTATATTTCCAACTTTGTTCAT
AGTTGGAGTTGGGGGCAGTAACTGACTCCCCCAAAAAAACCTATCTCCAGGCCCTAACATAAAGTTCAAA
AACAGCTGGAACCGAAAAAAAAAAAAAAAAAACAAATTAAGGCATTGTGCAGACAGCTGGTCATTCAAAT
CCCTTATGCTTTAGCTGTCAGCCAGTTGCCCGAATCCTCAGGCCCCCTTGTAATTAACACCAGGCTCCGT
ACAATTAGCCACAGGACCGGGTGCCAGAAACAGAGTCAATCTTCACAGTAGGAGGTAGGGAGAGGAATCA
TAAGGGGTCCTGGCCTTCACAGAGCACAGGACCAGGGTGGAGGAAAGGGCATGACCCAAAGGCTGCAGTA
TCAGGGAGGAGCAGGGTCTGTGTCATAGGAAAAAACAGGCCAAAGACTGTGAGCCTGTGGAATAAACTGC
AGTCACTTCCAGCTGAACTTGGCTGCAGAAGATAAGTTTTTGACTGTGGAGGTTGACCAAGAGGATATTC
TAGGCGGAGGCAAAGATAACATGGTAGGAAGGGTAAGACGGAGGAGGAAGAGAGGACTTATGCAATGGGC
ATTTACTGAGCCCCTACTTTGTGCTACCTACCATGCCAGGCTCTGAGGGCCCAGAGTCCTCAAATGTGGA
TGCCCAAGTGGCAGTCTATACCAAGCAAAGCAGAGTGTCATCACAGCCAACTTTGTCACGTCTCATGGCT
GGAGATACTAATTAGATAATTTCTTCCTTCTTTTAAAGGCCTGATTTTTTCTGCAATTCTTAAGTCACTT
CAAAGTTACTTCTCCCCAACGCTCTTCAGAGTCATCCGCCTGGCCCGAATTGGCCGCATCCTCAGACTGA
TCCGAGCGGCCAAGGGGATCCGCACACTGCTCTTTGCCCTCATGATGTCCCTGCCTGCCCTCTTCAACAT
CGGGCTGTTGCTATTCCTTGTCATGTTCATCTACTCTATCTTCGGTATGTCCAGCTTTCCCCATGTGAGG
TGGGAGGCTGGCATCGACGACATGTTCAACTTCCAGACCTTCGCCAACAGCATGCTGTGCCTCTTCCAGA
TTACCACGTCGGCCGGCTGGGATGGCCTCCTCAGCCCCATCCTCAACACAGGGCCCCCCTACTGTGACCC
CAATCTGCCCAACAGCAATGGCACCAGAGGGGACTGTGGGAGCCCAGCCGTAGGCATCATCTTCTTCACC
ACCTACATCATCATCTCCTTCCTCATCGTGGTCAACATGTACATTGCAGTGATTCTGGAGAACTTCAATG
TGGCCACGGAGGAGAGCACTGAGCCCCTGAGTGAGGACGACTTTGACATGTTCTATGAGACCTGGGAGAA
GTTTGACCCAGAGGCCACTCAGTTTATTACCTTTTCTGCTCTCTCGGACTTTGCAGACACTCTCTCTGGT
CCCCTGAGAATCCCAAAACCCAATCGAAATATACTGATCCAGATGGACCTGCCTTTGGTCCCTGGAGATA
AGATCCACTGCTTGGACATCCTTTTTGCTTTCACCAAGAATGTCCTAGGAGAATCCGGGGAGTTGGATTC
TCTGAAGGCAAATATGGAGGAGAAGTTTATGGCAACTAATCTTTCAAAATCATCCTATGAACCAATAGCA
ACCACTCTCCGATGGAAGCAAGAAGACATTTCAGCCACTGTCATTCAAAAGGCCTATCGGAGCTATGTGC
TGCACCGCTCCATGGCACTCTCTAACACCCCATGTGTGCCCAGAGCTGAGGAGGAGGCTGCATCACTCCC
AGATGAAGGTTTTGTTGCATTCACAGCAAATGAAAATTGTGTACTCCCAGACAAATCTGAAACTGCTTCT
GCCACATCATTCCCACCGTCCTATGAGAGTGTCACTAGAGGCCTTAGTGATAGAGTCAACATGAGGACAT
CTAGCTCAATACAAAATGAAGATGAAGCCACCAGTATGGAGCTGATTGCCCCTGGGCCCTAGTGA

1. A gRNA comprising a spacer comprising the nucleotide sequence of any one of SEQ ID NOs:1-310.

2. The gRNA of claim 1, wherein the spacer consists of the nucleotide sequence of any one of SEQ ID NOs:1-310.

3. A nucleotide sequence encoding the gRNA of claim 1 or 2.

4. An AAV vector comprising the nucleotide sequence of claim 3.

5. An AAV vector comprising a nucleotide sequence encoding two or more gRNAs, wherein the two or more gRNAs comprise a spacer comprising the nucleotide sequence of any one of SEQ ID NOs:1-310.

6. An AAV vector comprising two or more nucleotide sequences, wherein the two or more nucleotide sequences encode two or more gRNAs comprising a spacer comprising the nucleotide sequence of any one of SEQ ID NOs:1-310.

7. The AAV vector of any one of claims 4-6, wherein the AAV vector further comprises a nucleotide sequence encoding an RNA-guided nuclease.

8. The AAV vector of any one of claims 5-7, comprising a first nucleotide sequence encoding a gRNA comprising a spacer comprising the nucleotide sequence of SEQ ID NO:5, and a second nucleotide sequence encoding a gRNA comprising a spacer comprising the nucleotide sequence of SEQ ID NO:189.

9. The AAV vector of any one of claims 5-7, comprising a first nucleotide sequence encoding a gRNA comprising a spacer comprising the nucleotide sequence of SEQ ID NO: 7, and a second nucleotide sequence encoding a gRNA comprising a spacer comprising the nucleotide sequence of SEQ ID NO: 182.

10. The AAV vector of any one of claims 5-7, comprising a first nucleotide sequence encoding a gRNA comprising a spacer comprising the nucleotide sequence of SEQ ID NO: 7, and a second nucleotide sequence encoding a gRNA comprising a spacer comprising the nucleotide sequence of SEQ ID NO: 189.

11. The AAV vector of any one of claims 5-7, comprising a first nucleotide sequence encoding a gRNA comprising a spacer comprising the nucleotide sequence of SEQ ID NO: 5, and a second nucleotide sequence encoding a gRNA comprising a spacer comprising the nucleotide sequence of SEQ ID NO: 182.

12. A cell comprising the AAV vector of any one of claims 4-11.

13. The cell of claim 12, wherein the cell is a neuronal cell or precursor thereof.

14. The cell of claim 13, wherein the neuronal cell is a peripheral neuronal cell or precursor thereof.

15. The cell of claim 14, wherein the peripheral neuronal cell is a dorsal root ganglion cell.

16. A composition comprising the gRNA of claim 1 or 2.

17. The composition of claim 16, further comprising an RNA-guided nuclease.

18. The composition of claim 17, comprising a complex of the gRNA and the RNA-guided nuclease.

19. The composition of claim 17, comprising an RNP complex of the gRNA and the RNA-guided nuclease.

20. A composition comprising the AAV vector of any one of claims 4-11.

21. The composition of claim 20, further comprising a second AAV vector comprising a nucleotide sequence encoding an RNA-guided nuclease.

22. A composition comprising the AAV vector of claim 7.

23. A method comprising:
administering to a subject in need thereof (i) a first nucleotide sequence that encodes a guide RNA (gRNA) that targets a gene encoding a voltage-gated sodium channel subunit, and (ii) a second nucleotide sequence that encodes an RNA-guided nuclease, wherein the gRNA comprises a spacer comprising or consisting of the nucleotide sequence of any one of SEQ ID NOs:1-310.

24. The method of claim 23, wherein the gRNA comprises a spacer comprising the nucleotide sequence of SEQ ID NO: 5.

25. The method of claim 23, wherein the gRNA comprises a spacer comprising the nucleotide sequence of SEQ ID NO: 7.

26. The method of claim 23, wherein the gRNA comprises a spacer comprising the nucleotide sequence of SEQ ID NO: 182.

27. The method of claim 23, wherein the gRNA comprises a spacer comprising the nucleotide sequence of SEQ ID NO: 189.

28. The method of any one of claims 23-27, further comprising administering to the subject a third nucleotide sequence that encodes a gRNA that targets a gene encoding a voltage-gated sodium channel subunit.

29. The method of claim 28, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:189.

30. The method of claim 28, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:182.

31. The method of claim 28, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:182.

32. The method of claim 28, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:189.

33. The method of any one of claims 23-27, comprising administering a vector comprising the first nucleotide sequence and/or the second nucleotide sequence.

34. The method of any one of claims 28 to 32, comprising administering a vector comprising the first nucleotide sequence, the second nucleotide sequence, and/or the third nucleotide sequence.

35. The method of claim 33 or 34, wherein the vector is an adeno associated virus (AAV) vector.

36. The method of claim 35, wherein the AAV vector comprises components from one serotype of AAV.

37. The method of claim 36, wherein the AAV vector comprises components from more than one serotype of AAV.

38. The method of claim 36 or 37, wherein the serotype is 1, 2, 3, 4, 5, 6, 7, 8, or 9.

39. The method of claim 36, wherein the serotype is 5.

40. The method of claim 36, wherein the serotype is 9.

41. A method comprising:
administering to a subject in need thereof a composition comprising (i) a gRNA that targets a gene encoding a voltage-gated sodium channel subunit, wherein the gRNA comprises a spacer comprising or consisting of the nucleotide sequence of any one of SEQ ID NOs:1-310; and (ii) an RNA-guided nuclease.

42. The method of claim 41, wherein the gRNA comprises a spacer comprising the nucleotide sequence of SEQ ID NO: 5.

43. The method of claim 41, wherein the gRNA comprises a spacer comprising the nucleotide sequence of SEQ ID NO: 7.

44. The method of claim 41, wherein the gRNA comprises a spacer comprising the nucleotide sequence of SEQ ID NO: 182.

45. The method of claim 41, wherein the gRNA comprises a spacer comprising the nucleotide sequence of SEQ ID NO: 189.

46. The method of claim 41, comprising administering a complex of the gRNA and the RNA-guided nuclease.

47. The method of claim 46, comprising administering an RNP complex of the gRNA and the RNA-guided nuclease.

48. The method of claim 41, wherein the composition comprises a gRNA comprising a spacer comprising the nucleotide sequence of SEQ ID NO:5, and a gRNA comprising a spacer comprising the nucleotide sequence of SEQ ID NO: 189.

49. The method of claim 41, wherein the composition comprises a gRNA comprising a spacer comprising the nucleotide sequence of SEQ ID NO:7, and a gRNA comprising a spacer comprising the nucleotide sequence of SEQ ID NO: 182.

50. The method of claim 41, wherein the composition comprises a gRNA comprising a spacer comprising the nucleotide sequence of SEQ ID NO:5, and a gRNA comprising a spacer comprising the nucleotide sequence of SEQ ID NO: 182.

51. The method of claim 41, wherein the composition comprises a gRNA comprising a spacer comprising the nucleotide sequence of SEQ ID NO:7, and a gRNA comprising a spacer comprising the nucleotide sequence of SEQ ID NO: 189.

52. A method of treating a subject for one or more symptoms associated with a pain disorder and/or disorder associated with pain, the method comprising:
administering to a subject in need thereof (i) a first nucleotide sequence that encodes a gRNA that targets a gene encoding a voltage-gated sodium channel subunit, and (ii) a second nucleotide sequence that encodes an RNA-guided nuclease, wherein the gRNA comprises a spacer comprising or consisting of the nucleotide sequence of one of SEQ ID NOs:1-310, e.g., wherein a level or measure of at least one symptom is reduced or ameliorated relative to control (e.g., a level or measure of the at least one symptom in the subject prior to the administering).

53. The method of claim 52, further comprising administering to the subject a third nucleotide sequence that encodes a gRNA that targets a gene encoding a voltage-gated sodium channel subunit.

54. The method of claim 53, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:189.

55. The method of claim 53, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:182.

56. The method of claim 53, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:182.

57. The method of claim 53, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:189.

58. The method of claim 52, comprising administering a vector comprising the first nucleotide sequence and/or the second nucleotide sequence.

59. The method of any one of claims 53 to 57, comprising administering a vector comprising the first nucleotide sequence, the second nucleotide sequence, and/or the third nucleotide sequence.

60. The method of claim 58 or 59, wherein the vector is an AAV vector.

61. The method of claim 60, wherein the AAV vector comprises components from one serotype of AAV.

62. The method of claim 60, wherein the AAV vector comprises components from more than one serotype of AAV.

63. The method of claim 61 or 62, wherein the serotype is 1, 2, 3, 4, 5, 6, 7, 8, or 9.

64. A method of treating a subject for one or more symptoms associated with a pain disorder and/or disorder associated with pain, the method comprising:
administering to a subject in need thereof a composition comprising (i) a gRNA that targets a gene encoding a voltage-gated sodium channel subunit, wherein the gRNA comprises a spacer comprising or consisting of the nucleotide sequence of any one of SEQ ID NOs:1-310; and (ii) an RNA-guided nuclease, wherein a level or measure of at least one symptom is reduced or ameliorated relative to control (e.g., a level or measure of the at least one symptom in the subject prior to the administering).

65. The method of claim 64, comprising administering a complex of the gRNA and the RNA-guided nuclease.

66. The method of claim 64, comprising administering an RNP complex of the gRNA and the RNA-guided nuclease.

67. The method of any one of claims 64-66, wherein the composition comprises a gRNA comprising a spacer comprising the nucleotide sequence of SEQ ID NO:5, and a gRNA comprising a spacer comprising the nucleotide sequence of SEQ ID NO:189.

68. The method of any one of claims 64-66, wherein the composition comprises a gRNA comprising a spacer comprising the nucleotide sequence of SEQ ID NO:7, and a gRNA comprising a spacer comprising the nucleotide sequence of SEQ ID NO:182.

69. The method of any one of claims 64-66, wherein the composition comprises a gRNA comprising a spacer comprising the nucleotide sequence of SEQ ID NO:5, and a gRNA comprising a spacer comprising the nucleotide sequence of SEQ ID NO:182.

70. The method of any one of claims 64-66, wherein the composition comprises a gRNA comprising a spacer comprising the nucleotide sequence of SEQ ID NO:7, and a gRNA comprising a spacer comprising the nucleotide sequence of SEQ ID NO:189.

71. A method of treating a subject, the method comprising:
administering to a neuronal cell of a subject in need thereof (i) a first nucleotide sequence that encodes a gRNA that targets a gene encoding a voltage-gated sodium channel subunit, and (ii) a second nucleotide sequence that encodes an RNA-guided nuclease, wherein the gRNA comprises a spacer comprising or consisting of the nucleotide sequence of one of SEQ ID NOs:1-310.

72. The method of claim 71, further comprising administering to the subject a third nucleotide sequence that encodes a gRNA that targets a gene encoding a voltage-gated sodium channel subunit.

73. The method of claim 72, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:189.

74. The method of claim 72, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:182.

75. The method of claim 72, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:182.

76. The method of claim 72, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:189.

77. The method of claim 71, wherein administering comprises contacting a neuronal cell with the first and/or second nucleotide sequences.

78. The method of any one of claims 72-76, wherein administering comprises contacting a neuronal cell with the first, second, and/or third nucleotide sequences.

79. The method of any one of claims 71-78, wherein the neuronal cell is a neuron or progenitor thereof.

80. The method of any one of claims 71-78, wherein the neuronal cell or progenitor is a dorsal root ganglion or dorsal root ganglion cell progenitor.

81. The method of claim 71, comprising administering a vector comprising the first nucleotide sequence and/or the second nucleotide sequence.

82. The method of any one of claims 72 to 80, comprising administering a vector comprising the first nucleotide sequence, the second nucleotide sequence, and/or the third nucleotide sequence.

83. The method of claim 81 or 82, wherein the vector is an AAV vector.

84. The method of claim 83, wherein the AAV vector comprises components from one serotype of AAV.

85. The method of claim 83, wherein the AAV vector comprises components from more than one serotype of AAV.

86. The method of claim 84 or 85, wherein the serotype is 1, 2, 3, 4, 5, 6, 7, 8, or 9.

87. A method comprising:
administering to a neuronal cell of a subject in need thereof (i) a gRNA that targets a gene encoding a voltage-gated sodium channel subunit, and (ii) an RNA-guided nuclease, wherein the gRNA comprises a spacer comprising or consisting of the nucleotide sequence of one of SEQ ID NOs:1-310.

88. The method of claim 87, comprising administering a complex of the gRNA and the RNA-guided nuclease.

89. The method of claim 87, comprising administering an RNP complex of the gRNA and the RNA-guided nuclease.

90. The method of any one of claims 87-89, wherein administering comprises contacting the neuronal cell with the gRNA and the RNA-guided nuclease.

91. The method of any one of claims 87-90, wherein the neuronal cell is a neuron or progenitor thereof.

92. The method of claim 91, wherein the neuronal cell or progenitor is a dorsal root ganglion or dorsal root ganglion cell progenitor.

93. The method of claim 92, wherein the administering occurs in-vivo directly to the central nervous system.

94. The method of any one of claims 71-93, wherein the neuronal cell has a genetic mutation within the SCN9A or SCN10A locus.

95. A method of editing a neuronal cell in a subject in need thereof, the method comprising:
administering to the subject (i) a first nucleotide sequence that encodes a gRNA that targets a gene encoding a voltage-gated sodium channel subunit, wherein the gRNA comprises a spacer comprising or consisting of the nucleotide sequence of any one of SEQ ID NOs:1-310; and (ii) a second nucleotide sequence that encodes an RNA-guided nuclease, wherein upon expression of the gRNA and the RNA-guided nuclease, the nuclease creates one or more single- or double-strand breaks within the gene or within a regulatory element of the gene.

96. The method of claim 95, further comprising administering to the subject a third nucleotide sequence that encodes a gRNA that targets a gene encoding a voltage-gated sodium channel subunit.

97. The method of claim 96, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:189.

98. The method of claim 96, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:182.

99. The method of claim 96, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:182.

100. The method of claim 96, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:189.

101. The method of any one of claims 95-100, wherein the one or more breaks results in one or more nucleotide changes in the gene or regulatory element of the gene.

102. The method of any one of claims 95-101, wherein level of expression of the gene is changed relative to control.

103. The method of any one of claims 95-102, wherein the gene is SCN9A and/or SCN10A.

104. The method of any one of claims 95-103, wherein the one or more changes is a permanent insertion, deletion, and/or substitution of at least one nucleotide within or near the SCN9A and/or SCN10A gene(s) or regulatory element(s).

105. The method of claim 102, wherein the change in gene expression is a reduction in expression.

106. The method of claim 103, wherein the level of SCN9A and/or SCN10A gene expression is reduced as compared to control.

107. The method of any one of claims 95-106, comprising administering a vector comprising the first nucleotide sequence, the second nucleotide sequence, and/or the third nucleotide sequence.

108. The method of claim 107, wherein the vector is an AAV vector.

109. The method of claim 108, wherein the AAV vector comprises components from one serotype of AAV.

110. The method of claim 108, wherein the AAV vector comprises components from more than one serotype of AAV.

111. The method of claim 108 or 109, wherein the serotype is 1, 2, 3, 4, 5, 6, 7, 8, or 9.

112. A method of editing a neuronal cell in a subject in need thereof comprising:
administering to the subject (i) a first nucleotide sequence that encodes a gRNA that targets an SCN9A and/or SCN10A gene or regulatory element, wherein the gRNA comprises a spacer comprising or consisting of the nucleotide sequence of any one of SEQ ID NOs:1-310; and (ii) a second nucleotide sequence that encodes an RNA-guided nuclease, wherein upon expression of the gRNA and the RNA-guided nuclease, the nuclease creates one or more single- or double-strand breaks within the SCN9A or SCN10A genes or regulatory element.

113. A method of editing a neuronal cell in a subject in need thereof comprising:
administering directly to the dorsal root ganglion neurons of the subject (i) an AAV particle comprising a first nucleotide sequence that encodes at least one gRNA that targets an SCN9A and/or SCN10A gene or regulatory element, wherein the gRNA comprises a spacer comprising the nucleotide sequence of any one of SEQ ID NOs:1-310; and (ii) a second nucleotide sequence that encodes an RNA-guided nuclease, wherein upon expression of the gRNA and the RNA-guided nuclease, the nuclease creates one or more single- or double-strand breaks within the SCN9A or SCN10A genes or regulatory element.

114. The method of claim 113, wherein an AAV vector serotype is AAV5, or AAV9.

115. The method of claim 113 or 114, wherein the gRNA comprises a spacer comprising the nucleotide sequence of SEQ ID NO: 5.

116. The method of claim 113 or 114, wherein the gRNA comprises a spacer comprising the nucleotide sequence of SEQ ID NO: 7.

117. The method of claim 113 or 114, wherein the gRNA comprises a spacer comprising the nucleotide sequence of SEQ ID NO: 182.

118. The method of claim 113 or 114, wherein the gRNA comprises a spacer comprising the nucleotide sequence of SEQ ID NO: 189.

119. The method of claim 113 or 114, wherein the AAV vector comprises a first nucleotide sequence encoding a gRNA comprising a spacer comprising the nucleotide sequence of SEQ ID NO:5, and a third nucleotide sequence encoding a gRNA comprising a spacer comprising the nucleotide sequence of SEQ ID NO:189.

120. The method of claim 113 or 114, wherein the AAV vector comprises a first nucleotide sequence encoding a gRNA comprising a spacer comprising the nucleotide sequence of SEQ ID NO:7, and a third nucleotide sequence encoding a gRNA comprising a spacer comprising the nucleotide sequence of SEQ ID NO:182.

121. The method of claim 113 or 114, wherein the AAV vector comprises a first nucleotide sequence encoding a gRNA comprising a spacer comprising the nucleotide sequence of SEQ ID NO:5, and a third nucleotide sequence encoding a gRNA comprising a spacer comprising the nucleotide sequence of SEQ ID NO:182.

122. The method of claim 113 or 114, wherein the AAV vector comprises a first nucleotide sequence encoding a gRNA comprising a spacer comprising the nucleotide sequence of SEQ ID NO:7, and a third nucleotide sequence encoding a gRNA comprising a spacer comprising the nucleotide sequence of SEQ ID NO:189.

123. A method of treating a subject, the method comprising:
(i) administering to an autologous iPSC cell (a) a first nucleotide sequence that encodes a gRNA that targets a gene encoding a voltage-gated sodium channel subunit, wherein the gRNA comprises a spacer comprising or consisting of the nucleotide sequence of any one of SEQ ID NOs:1-310; and (b) a second nucleotide sequence that encodes an RNA-guided nuclease;
(ii) differentiating the iPSC cell into a neuron or neuronal progenitor; and
(iii) transplanting the differentiated cell into a subject.

124. The method of claim 123, further comprising administering to the autologous iPSC cell a third nucleotide sequence that encodes a gRNA that targets a gene encoding a voltage-gated sodium channel subunit.

125. The method of claim 124, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:189.

126. The method of claim 124, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:182.

127. The method of claim 124, wherein the first nucleotide sequence encodes a gRNA comprising a spacer comprising the nucleotide sequence of SEQ ID NO:5, and the third nucleotide sequence encodes a gRNA comprising a spacer comprising the nucleotide sequence of SEQ ID NO:182.

128. The method of claim 124, wherein the first nucleotide sequence encodes a gRNA comprising a spacer comprising the nucleotide sequence of SEQ ID NO:7, and the third nucleotide sequence encodes a gRNA comprising a spacer comprising the nucleotide sequence of SEQ ID NO:189.

129. A gRNA that targets a PAM that is targeted by a gRNA comprising a spacer comprising the nucleotide sequence of any one of SEQ ID NOs:1-310.

130. A gRNA that targets (i) a target sequence of a gene encoding a voltage-gated sodium channel subunit or (ii) a target sequence of a regulatory element of the gene, wherein the target sequence is targeted by a gRNA comprising a spacer comprising the nucleotide sequence of any one of SEQ ID NOs:1-310.

131. A composition comprising a gRNA comprising a spacer comprising the nucleotide sequence of SEQ ID NO:5, and gRNA comprising a spacer comprising the nucleotide sequence of SEQ ID NO:189.

132. A composition comprising a gRNA comprising a spacer comprising the nucleotide sequence of SEQ ID NO:7, and gRNA comprising a spacer comprising the nucleotide sequence of SEQ ID NO:182.

133. A composition comprising a gRNA comprising a spacer comprising the nucleotide sequence of SEQ ID NO:5, and a gRNA comprising a spacer comprising the nucleotide sequence of SEQ ID NO:182.

134. A composition comprising a gRNA comprising a spacer comprising the nucleotide sequence of SEQ ID NO:7, and a gRNA comprising a spacer comprising the nucleotide sequence of SEQ ID NO:189.

135. A composition comprising a first nucleotide sequence encoding a gRNA comprising a spacer comprising the nucleotide sequence of SEQ ID NO:5, and a second nucleotide sequence encoding a gRNA comprising a spacer comprising the nucleotide sequence of SEQ ID NO:189.

136. A composition comprising a first nucleotide sequence encoding a gRNA comprising a spacer comprising the nucleotide sequence of SEQ ID NO:7, and a second nucleotide sequence encoding a gRNA comprising a spacer comprising the nucleotide sequence of SEQ ID NO:182.

137. A composition comprising a first nucleotide sequence encoding a gRNA comprising a spacer comprising the nucleotide sequence of SEQ ID NO:5, and a second nucleotide sequence encoding a gRNA comprising a spacer comprising the nucleotide sequence of SEQ ID NO:182.

138. A composition comprising a first nucleotide sequence encoding a gRNA comprising a spacer comprising the nucleotide sequence of SEQ ID NO:7, and a second nucleotide sequence encoding a gRNA comprising a spacer comprising the nucleotide sequence of SEQ ID NO:189.

139. The method of any one of claims 23-128, wherein the subject has or is at risk of a pain disorder and/or disorder associated with pain.

140. The method of any one of claims 23-128 or 139, wherein the subject has a genetic mutation within the SCN9A and/or SCN10A locus.

141. The method of claim 139 or 140, wherein the pain disorder is nociceptive and/or neuropathic pain.
